(19) United States
(12) Patent Application Publication
(10) Pub. No.: US 2025/0282795 A1
(43) Pub. Date: Sep. 11, 2025

(54) EGFR Inhibitors in cancer treatment

(71) Applicant: Celyn Therapeutics, Inc., San Diego, CA (US)

(72) Inventors: Ruben Abagyan, La Jolla, CA (US); Natalia I Vasilevich, Moscow (RU); Alexander Khvat, San Diego, CA (US); Alexandre Vasilievich Ivachtchenko, Hallandale Beach, FL (US); Nikolay Savchuk, San Diego, CA (US)

(21) Appl. No.: 18/858,316

(22) PCT Filed: Apr. 15, 2023
(86) PCT No.: PCT/US2023/065829

§ 371 Date: Oct. 20, 2024

Related U.S. Application Data
(60) Provisional application No. 63/332,835, filed on Apr. 20, 2022.

(51) Int. Cl. C07D 498/22; A61K 31/439; A61K 31/4995; A61K 31/5386; A61P 35/00; C07D 471/22

(52) U.S. Cl. CPC C07D 498/22; A61K 31/439; A61K 31/4995; A61K 31/5386; A61P 35/00; C07D 471/22

(57) ABSTRACT

The present invention is directed to the compounds of Formula (A)—inhibitors of EGFR. The inhibitors described herein can be useful in the treatment of diseases or disorders associated with EGFR, such as is lung cancer. In particular, the invention is concerned with compounds and pharmaceutical compositions inhibiting EGFR kinase activity in a cell, methods of treating diseases or disorders associated with EGFR, and methods of synthesizing these compounds.

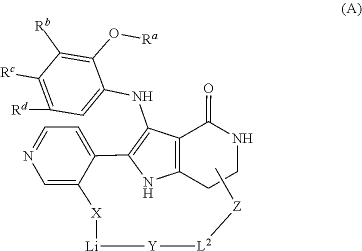

### CROSS-REFERENCE TO RELATED APPLICATIONS

This application claims priority to and benefit of U.S. Provisional Patent Application Ser. No. 63/332,835 filed Apr. 20, 2022, entitled “EGFR Inhibitors in Cancer Treatment”, the disclosure of which is incorporated by reference in its entirety for all purposes.

### FIELD OF INVENTION

The present invention is directed to inhibitors of Epidermal Growth Factor Receptor (EGFR or EGF-receptor)—receptor of tyrosine kinase family. The inhibitors described herein can be useful in the treatment of diseases or disorders associated with EGFR, such as cancer. In particular, the invention is concerned with compounds and pharmaceutical compositions inhibiting EGFR, methods of treating diseases or disorders associated with EGFR, and methods of synthesizing these compounds.

### BACKGROUND

The physiological function of the epidermal growth factor receptor (EGFR) is to regulate epithelial tissue development and homeostasis. The epidermal growth factor receptor (EGFR) is a tyrosine kinase receptor for various growth factors including EGF (epidermal growth factor), TGF-α (transforming growth factor-α) and other EGF-ike ligands. The EGFR pathway plays an important role in pulmonary physiology especially the function of epithelial cells via signaling transduction that regulates key cellular processes such as self-renew, wound-heaiing, proliferation, survival, adhesion, migration, and differentiation. Epidermal growth factor receptor (EGFR) is a transmembrane protein with cytoplasmic kinase activity that transduces important growth factor signaling from the extracellular milieu to the cell.

EGFR gene amplification, overexpression, and mutation are frequently observed in various cancer indications and are associated with a poor prognosis. The epidermal growth factor receptor (EGFR) over-activation is observed in a vast number of cancers.

In pathological settings, mostly in lung and breast cancer and in glioblastoma, the EGFR is a driver of tumorigenesis. Inappropriate activation of the EGFR in cancer mainly results from amplification and point mutations at the genomic locus, but transcriptional upregulation or ligand overproduction due to autocrine/paracrine mechanisms has also been described. Moreover, the EGFR is increasingly recognized as a biomarker of resistance in tumors, as its amplification or secondary mutations have been found to arise under drug pressure. This evidence, in addition to the prominent function that this receptor plays in normal epithelia, has prompted intense investigations into the role of the EGFR both at physiological and at pathological level.

High abundance of EGFR and large internal deletions are frequently observed in brain tumors, whereas point mutations and small insertions within the kinase domain are common in lung cancer.

Given that more than 60% of non-small cell lung carcinomas (NSCLCs) express EGFR, EGFR has become an important therapeutic target for the treatment of these tumors. EGFR inhibitors have been widely used in treatment of non-small cell lung cancer (NSCLC).

Several reports have been published recently demonstrating a beneficial effect of epidermal growth factor receptor (EGFR) inhibitors in improving pathological and behavioral conditions in neurodegenerative diseases (NDDs) such as Alzheimer's disease (AD) and Amyotrophic Lateral Sclerosis (ALS) as well as the brain and spinal cord injuries (SCI).

In summary, EGFR is a promising drug target for cancer therapy. Targeting EGFR and its downstream signaling cascades are regarded as a rational and valuable approach in cancer therapy.

Several synthetic EGFR tyrosine kinase inhibitors (TKIs) have been evaluated in recent years, mostly exhibited clinical efficacy in relevant models and categorized into first, second, third and fourth generation. However, studies are still ongoing to find more efficient EGFR inhibitors in light of the resistance to the current inhibitors.

EGFR inhibitors have produced impressive therapeutic benefits to responsive types of cancers. Unfortunately, however, patients who initially respond to anti-EGFR drugs almost inevitably develop resistance. Resolving mechanisms of drug resistance translates to higher granularity maps of signaling pathways and it holds the promise of prolonging patient response. A novel targeted therapy that is able to specifically address the EGFR-C797S acquired resistance mutation would be highly beneficial for those patients.

In particular, patients with primary resistance to approved anti-EGFR therapies, due to EGFR exon20 insertions, have only few treatment options to date and there is a great need for novel alternative and/or improved therapeutics to provide these patients with an efficacious, well tolerable therapy. Therefore, potent inhibitors of mutant EGFR, particularly of mutant EGFR with exon20 insertion mutations that show improved selectivity versus wild-type EGFR, represent valuable compounds that should complement therapeutic options either as single agents or in combination with other drugs.

### SUMMARY

A first aspect of the invention relates to compounds of Formula (A):

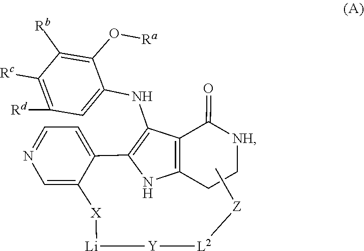

or a pharmaceutically acceptable salt, prodrug, stereoisomer, solvate, or tautomer thereof, wherein
R^{a} is selected from H, C₁-C₆ alkyl, C₂-C₆ alkenyl, C₂-C₆ alkynyl, C₃-C₁₀ cycloalkyl, heterocyclyl, aryl, and heteroaryl, wherein the alkyl, alkenyl, alkynyl, cycloalkyl, heterocyclyl, aryl, or heteroaryl is optionally substituted with one or more substituents independently selected from halogen, OH, ═O, NR³R⁴, CN, NO₂, COOR¹, CONR³R⁴;
R^{b}, R^{c}, R^{d} are each independently selected from H, halogen, OH, NH₂, CN, NO₂, C₁-C₆ alkyl, and C₁-C₆ alkoxy, wherein alkyl or alkoxy is optionally substituted with one or more halogen, CN;
X is selected from a bond, O, NR¹, C(R²)₂, S, S(O), S(O)₂, C(O), C(O)O, C(O)NR¹, OC(O)O, N(R²)C(O)NR², arenediyl;
R¹ is selected from H, C₁-C₆ alkyl, C₃-C₁₀ cycloalkyl, C₂-C₆ alkenyl, C₂-C₆ alkynyl, C₁-C₆ acyl, heterocycle, aryl, heteroaryl, and S(O)₂R² wherein the alkyl, alkenyl, alkynyl, heterocycle, cycloalkyl, aryl, or heteroaryl is optionally substituted with one or more substituents independently selected from halogen, OH, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, heteroaryl;
each R² is independently selected from H, C₁-C₆ alkyl, C₃-C₁₀ cycloalkyl, C₂-C₆ alkenyl, C₂-C₆ alkynyl, heterocycle, aryl, and heteroaryl wherein the alkyl, alkenyl, alkynyl, heterocycle, cycloalkyl, aryl, or heteroaryl is optionally substituted with one or more substituents independently selected from halogen, OH, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, heteroaryl;
Y is selected from a bond, O, NR¹, C(R²)₂, S, S(O), S(O)₂, C(O), C(O)O, C(O)NR¹, OC(O)O, N(R²)C(O)NR²;
Z is selected from a bond, O, C(R²)₂;
L¹ is selected from heterocyclediyl-C₁-C₁₀-alkanediyl, C₁-C₁₀ alkanediyl, C₂-C₁₀ alkenediyl, C₂-C₁₀ alkynediyl, C₃-C₁₀-cycloalkanediyl, C₃-C₁₀-cycloalkanediyl-C₁-C₁₀-alkanediyl, arenediyl, arenediyl-C₁-C₁₀-alkanediyl, heterocyclediyl, heterocyclediyl-C(O), heteroarenediyl, heteroarenediyl-C₁-C₁₀ alkanediyl, C₁-C₆ alkanediyl-O—C₁-C₆ alkanediyl, C₁-C₆ alkanediyl-NR¹—C₁-C₆ alkanediyl, C₁-C₆ alkanediyl-C(O)NR¹—C₁-C₆ alkanediyl, wherein alkanediyl, alkendiyl, alkynediyl, arenediyl, heterocyclediyl or heteroarenediyl heteroaryl is optionally substituted with one or more substituents independently selected from halogen, OH, oxo, CN, C₁-C₆ alkyl, C₁-C₆ alkoxy, C₁-C₆ alkyl-C₁-C₆ alkoxy, C₁-C₆ alkyl-NR¹—C₁-C₆ alkyl, NR³R⁴, —C(O)NR³R⁴, —S(O)C₁-C₆ alkyl, —S(O)₂NR³R⁴, aryl, and —S(O)₂C₁-C₆ alkyl;
L² is selected from C₁-C₁₀ alkanediyl, C₂-C₁₀ alkenediyl, C₂-C₁₀ alkynediyl, C₃-C₁₀-cycloalkanediyl, C₃-C₁₀-cycloalkanediyl-C₁-C₁₀-alkanediyl, arenediyl, arenediyl-C₁-C₁₀-alkanediyl, heterocyclediyl, heterocyclediyl-C₁-C₁₀-alkanediyl, heterocyclediyl-C(O), heteroarenediyl, heteroarenediyl-C₁-C₁₀ alkanediyl, C₁-C₆ alkanediyl-O—C₁—C₆ alkanediyl, C₁-C₆ alkanediyl-NR¹—C₁-C₆ alkanediyl, C₁-C₆ alkanediyl-C(O)NR¹—C₁-C₆ alkanediyl, wherein alkanediyl, alkendiyl, alkynediyl, arenediyl, heterocyclediyl or heteroarenediyl heteroaryl is optionally substituted with one or more substituents independently selected from halogen, OH, oxo, CN, C₁-C₆ alkyl, C₁-C₆ alkoxy, C₁-C₆ alkyl-C₁-C₆ alkoxy, C₁-C₆ alkyl-NR¹—C₁-C₆ alkyl, NR³R⁴, —C(O)NR³R⁴, —S(O)C₁-C₆ alkyl, —S(O)₂NR³R⁴, aryl, and —S(O)₂C₁-C₆ alkyl;
R³ and R⁴ are each independently selected from H, C₁-C₆ alkyl, C₁-C₆ acyl and C₃-C₁₀ cycloalkyl;
or R³ and R⁴ together with the atoms to which they are attached and any intervening atoms, form a 5-14 membered heterocycle, wherein the heterocycle is optionally substituted with one or more substituents independently selected from halogen, OH, ═O, CN, NO₂, C₁-C₆ alkyl, C₁-C₆ alkoxy;
   wherein,
aryl is cyclic, aromatic hydrocarbon groups that have 1 to 3 aromatic rings;
arenediyl is an organic radical derived from an aromatic divalent radical by removal of two hydrogens;
heterocyclyl is saturated or partially unsaturated 3-10 membered monocyclic, 7-12 membered bicyclic (fused, bridged, or spiro rings), or 11-14 membered tricyclic ring system (fused, bridged, or spiro rings) having one or more heteroatoms selected from O, N, S, P, Se, or B;
heterocyclediyl is a divalent functional group derived from heterocycle by the removal of two hydrogen atoms from ring atoms;
heteroaryl is a monovalent monocyclic or a polycyclic aromatic radical of 5 to 24 ring atoms, containing one or more ring heteroatoms selected from N, O, S, P, or B, the remaining ring atoms being C;
heteroarenediyl is a divalent functional group derived from heteroarenes by the removal of two hydrogen atoms from ring atoms.

Another aspect of the invention relates to compounds of Formula (I):

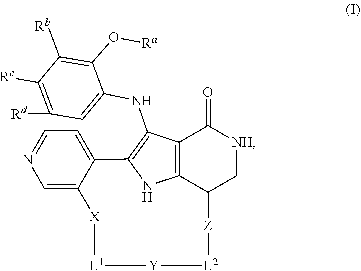

or a pharmaceutically acceptable salt, prodrug, stereoisomer, solvate, or tautomer thereof.

Another aspect of the invention relates to compounds of Formula (II):

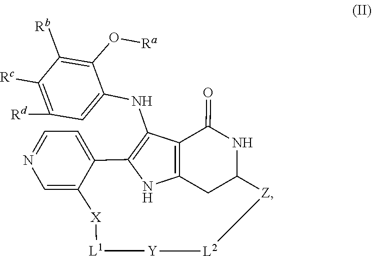

or a pharmaceutically acceptable salt, prodrug, stereoisomer, solvate, or tautomer thereof.

Another aspect of the invention is directed to pharmaceutical compositions comprising a compound of Formula (A), or a pharmaceutically acceptable salt, hydrate, solvate, prodrug, stereoisomer, or tautomer thereof and a pharmaceutically acceptable carrier. The pharmaceutical acceptable carrier may further include an excipient, diluent, or surfactant.

Another aspect of the invention is directed to pharmaceutical compositions comprising a compound of Formula (I), or a pharmaceutically acceptable salt, hydrate, solvate, prodrug, stereoisomer, or tautomer thereof and a pharmaceutically acceptable carrier. The pharmaceutical acceptable carrier may further include an excipient, diluent, or surfactant.

Another aspect of the invention is directed to pharmaceutical compositions comprising a compound of Formula (II), or a pharmaceutically acceptable salt, hydrate, solvate, prodrug, stereoisomer, or tautomer thereof and a pharmaceutically acceptable carrier. The pharmaceutical acceptable carrier may further include an excipient, diluent, or surfactant.

Another aspect of the invention relates to a method of treating a disease or disorder associated with EGFR. The method comprises administering to a patient in need of a treatment for diseases or disorders associated with EGFR an effective amount of a compound of Formula (A), or a pharmaceutically acceptable salt, hydrate, solvate, prodrug, stereoisomer, tautomer, or pharmaceutical composition thereof.

Another aspect of the invention relates to a method of treating a disease or disorder associated with EGFR. The method comprises administering to a patient in need of a treatment for diseases or disorders associated with EGFR an effective amount of a compound of Formula (I), or a pharmaceutically acceptable salt, hydrate, solvate, prodrug, stereoisomer, tautomer, or pharmaceutical composition thereof.

Another aspect of the invention relates to a method of treating a disease or disorder associated with EGFR. The method comprises administering to a patient in need of a treatment for diseases or disorders associated with EGFR an effective amount of a compound of Formula (II), or a pharmaceutically acceptable salt, hydrate, solvate, prodrug, stereoisomer, tautomer, or pharmaceutical composition thereof.

Another aspect of the invention is directed to a method of inhibiting of EGFR. The method involves administering to a patient in need thereof an effective amount of a compound of Formula (A), or a pharmaceutically acceptable salt, hydrate, solvate, prodrug, stereoisomer, tautomer, or pharmaceutical composition thereof.

Another aspect of the invention is directed to a method of inhibiting of EGFR. The method involves administering to a patient in need thereof an effective amount of a compound of Formula (I), or a pharmaceutically acceptable salt, hydrate, solvate, prodrug, stereoisomer, tautomer, or pharmaceutical composition thereof.

Another aspect of the invention is directed to a method of inhibiting of EGFR. The method involves administering to a patient in need thereof an effective amount of a compound of Formula (II), or a pharmaceutically acceptable salt, hydrate, solvate, prodrug, stereoisomer, tautomer, or pharmaceutical composition thereof.

Another aspect of the present invention relates to compounds of Formula (A), or pharmaceutically acceptable salts, hydrates, solvates, prodrugs, stereoisomers, tautomers, or pharmaceutical compositions thereof, for use in the manufacture of a medicament for inhibiting EGFR.

Another aspect of the present invention relates to compounds of Formula (I), or pharmaceutically acceptable salts, hydrates, solvates, prodrugs, stereoisomers, tautomers, or pharmaceutical compositions thereof, for use in the manufacture of a medicament for inhibiting EGFR.

Another aspect of the present invention relates to compounds of Formula (II), or pharmaceutically acceptable salts, hydrates, solvates, prodrugs, stereoisomers, tautomers, or pharmaceutical compositions thereof, for use in the manufacture of a medicament for inhibiting EGFR.

Another aspect of the present invention relates to the use of compounds of Formula (A), or pharmaceutically acceptable salts, hydrates, solvates, prodrugs, stereoisomers, tautomers, or pharmaceutical compositions thereof, in the treatment of diseases and disorders associated with EGFR.

Another aspect of the present invention relates to the use of compounds of Formula (I), or pharmaceutically acceptable salts, hydrates, solvates, prodrugs, stereoisomers, tautomers, or pharmaceutical compositions thereof, in the treatment of diseases and disorders associated with EGFR.

Another aspect of the present invention relates to the use of compounds of Formula (II), or pharmaceutically acceptable salts, hydrates, solvates, prodrugs, stereoisomers, tautomers, or pharmaceutical compositions thereof, in the treatment of diseases and disorders associated with EGFR.

Another aspect of the present invention relates to compounds of Formula (A), or pharmaceutically acceptable salts, hydrates, solvates, prodrugs, stereoisomers, tautomers, or pharmaceutical compositions thereof, for use in the manufacture of a medicament for treating or preventing a disease or disorder disclosed herein.

Another aspect of the present invention relates to compounds of Formula (I), or pharmaceutically acceptable salts, hydrates, solvates, prodrugs, stereoisomers, tautomers, or pharmaceutical compositions thereof, for use in the manufacture of a medicament for treating or preventing a disease or disorder disclosed herein.

Another aspect of the present invention relates to compounds of Formula (II), or pharmaceutically acceptable salts, hydrates, solvates, prodrugs, stereoisomers, tautomers, or pharmaceutical compositions thereof, for use in the manufacture of a medicament for treating or preventing a disease or disorder disclosed herein.

Another aspect of the invention is directed to a method of treating or preventing a disease or disorder disclosed herein in a subject in need thereof. The method involves administering to a patient in need of the treatment an effective amount of a compound of Formula (A), or a pharmaceutically acceptable salt, hydrate, solvate, prodrug, stereoisomer, tautomer, or pharmaceutical composition thereof.

Another aspect of the invention is directed to a method of treating or preventing a disease or disorder disclosed herein in a subject in need thereof. The method involves administering to a patient in need of the treatment an effective amount of a compound of Formula (I), or a pharmaceutically acceptable salt, hydrate, solvate, prodrug, stereoisomer, tautomer, or pharmaceutical composition thereof.

Another aspect of the invention is directed to a method of treating or preventing a disease or disorder disclosed herein in a subject in need thereof. The method involves administering to a patient in need of the treatment an effective amount of a compound of Formula (II), or a pharmaceutically acceptable salt, hydrate, solvate, prodrug, stereoisomer, tautomer, or pharmaceutical composition thereof.

Another aspect of the present invention relates to the use of compounds of Formula (A), or pharmaceutically acceptable salts, hydrates, solvates, prodrugs, stereoisomers, tautomers, or pharmaceutical compositions thereof, in the treatment of a disease or disorder disclosed herein.

Another aspect of the present invention relates to the use of compounds of Formula (I), or pharmaceutically acceptable salts, hydrates, solvates, prodrugs, stereoisomers, tautomers, or pharmaceutical compositions thereof, in the treatment of a disease or disorder disclosed herein.

Another aspect of the present invention relates to the use of compounds of Formula (II), or pharmaceutically acceptable salts, hydrates, solvates, prodrugs, stereoisomers, tautomers, or pharmaceutical compositions thereof, in the treatment of a disease or disorder disclosed herein.

The present invention further provides methods of treating a disease or disorder associated with EGFR, comprising administering to a patient suffering from at least one of said diseases or disorders a compound of Formula (A), or a pharmaceutically acceptable salt, hydrate, solvate, prodrug, stereoisomer, tautomer, or pharmaceutical composition thereof.

The present invention further provides methods of treating a disease or disorder associated with EGFR, comprising administering to a patient suffering from at least one of said diseases or disorders a compound of Formula (I), or a pharmaceutically acceptable salt, hydrate, solvate, prodrug, stereoisomer, tautomer, or pharmaceutical composition thereof.

The present invention further provides methods of treating a disease or disorder associated with EGFR, comprising administering to a patient suffering from at least one of said diseases or disorders a compound of Formula (II), or a pharmaceutically acceptable salt, hydrate, solvate, prodrug, stereoisomer, tautomer, or pharmaceutical composition thereof.

The present invention provides inhibitors of EGFR that are therapeutic agents in the treatment of diseases and disorders.

The present invention further provides compounds and compositions with an improved efficacy and safety profile relative to known inhibitors of EGFR. The present disclosure also provides agents with novel mechanisms of action toward EGFR in the treatment of various types of diseases.

The present invention further provides methods of treating a disease or disorder associated with EGFR, comprising administering to a patient suffering from at least one of said diseases or disorders a compound of Formula (A), or a pharmaceutically acceptable salt, hydrate, solvate, prodrug, stereoisomer, tautomer, or pharmaceutical composition thereof.

The present invention further provides methods of treating a disease or disorder associated with EGFR, comprising administering to a patient suffering from at least one of said diseases or disorders a compound of Formula (I), or a pharmaceutically acceptable salt, hydrate, solvate, prodrug, stereoisomer, tautomer, or pharmaceutical composition thereof.

The present invention further provides methods of treating a disease or disorder associated with EGFR, comprising administering to a patient suffering from at least one of said diseases or disorders a compound of Formula (II), or a pharmaceutically acceptable salt, hydrate, solvate, prodrug, stereoisomer, tautomer, or pharmaceutical composition thereof.

The present invention provides inhibitors of EGFR that are therapeutic agents in the treatment of diseases and disorders.

The present invention further provides methods of treating a disease, disorder, or condition selected from Inflammatory Skin and Bowel Disease, Neonatal, 2 (NISBD2); Lung Cancer; Lung Cancer Susceptibility 3 (LNCR3); Lung Squamous Cell Carcinoma; Gliosarcoma; Brain Stem Glioma; Adenocarcinoma; Colorectal Cancer; Glioblastoma; Breast Cancer; Squamous Cell Carcinoma; Small Cell Cancer of the Lung; Lung Squamous Cell Carcinoma; Inflammatory Skin and Bowel Disease, Neonatal, 2; Gastric Cancer; Prostate Cancer; Squamous Cell Carcinoma, Head and Neck; Pancreatic Cancer; Brain Cancer; Ovarian Cancer; Esophageal Cancer; Giant Cell Glioblastoma; Gliosarcoma; Lung Benign Neoplasm; Glioma; Exanthem; Endometrial Cancer; Adenoid Cystic Carcinoma; Bladder Cancer; Cowden Syndrome 1; Bronchiolo-Alveolar Adenocarcinoma; Oligodendroglioma; Hepatocellular Carcinoma; Lung Non-Squamous Non-Small Cell Carcinoma; High Grade Glioma; Glial Tumor; Laryngeal Squamous Cell Carcinoma; Renal Cell Carcinoma, Nonpapillary; Chronic Kidney Disease; Nasopharyngeal Carcinoma; Oral Squamous Cell Carcinoma; Lung Disease; Interstitial Lung Disease; Adenosquamous Lung Carcinoma; Bile Duct Cancer; Meningioma, Familial; Small Cell Carcinoma; Tumor Predisposition Syndrome; Pancreatic Adenocarcinoma; Diarrhea; Breast Ductal Carcinoma.

In some aspects, the present disclosure provides a compound obtainable by, or obtained by, a method for preparing compounds described herein (e.g., a method comprising one or more steps described in General Procedure).

In some aspects, the present disclosure provides an intermediate as described herein, being suitable for use in a method for preparing a compound as described herein (e.g., the intermediate is selected from the intermediates described in Preparative part—P1-P79).

In some aspects, the present disclosure provides a method of preparing compounds of the present disclosure.

In some aspects, the present disclosure provides a method of preparing compounds of the present disclosure, comprising one or more steps described herein.

In some specific non-limiting aspects, the present disclosure provides methods of preparing compounds of the present disclosure, described in the examples 1-20.

Unless otherwise defined, all technical and scientific terms used herein have the same meaning as commonly understood by one of ordinary skill in the art to which this disclosure belongs. In the specification, the singular forms also include the plural unless the context clearly dictates otherwise. Although methods and materials similar or equivalent to those described herein can be used in the practice or testing of the present disclosure, suitable methods and materials are described below. All publications, patent applications, patents and other references mentioned herein are incorporated by reference. The references cited herein are not admitted to be prior art to the claimed invention. In the case of conflict, the present specification, including definitions, will control. In addition, the materials, methods, and examples are illustrative only and are not intended to be limiting. In the case of conflict between the chemical structures and names of the compounds disclosed herein, the chemical structures will control.

Other features and advantages of the disclosure will be apparent from the following detailed description and claims

### DETAILED DESCRIPTION

The present disclosure provides methods of treating, preventing, or ameliorating a disease or disorder in which associated with the inhibition EGFR by administering to a subject in need thereof a therapeutically effective amount of a compound as disclosed herein.

The details of the disclosure are set forth in the accompanying description below. Although methods and materials similar or equivalent to those described herein can be used in the practice or testing of the present disclosure, illustrative methods and materials are now described. Other features, objects, and advantages of the disclosure will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this disclosure belongs. All patents and publications cited in this specification are incorporated herein by reference in their entireties.

### Definitions

The articles “a” and “an” are used in this disclosure to refer to one or more than one (i.e., to at least one) of the grammatical object of the article. By way of example, “an element” means one element or more than one element.

The term “and/or” is used in this disclosure to mean either “and” or “or” unless indicated otherwise.

The term “optionally substituted” is understood to mean that a given chemical moiety (e.g., an alkyl group) can (but is not required to) be bonded other substituents (e.g., heteroatoms). For instance, an alkyl group that is optionally substituted can be a fully saturated alkyl chain (i.e., a pure hydrocarbon). Alternatively, the same optionally substituted alkyl group can have one or more substituents different from hydrogen. For instance, it can, at any point along the chain be bounded to a halogen atom, a hydroxyl group, or any other substituent described herein. Thus, the term “optionally substituted” means that a given chemical moiety has the potential to contain other functional groups but does not necessarily have any further functional groups. Suitable substituents used in the optional substitution of the described groups include, without limitation, halogen, oxo, —OH, —CN, —COOH, —CH₂CN, —O—(C₁-C₆)alkyl, (C₁-C₆)alkyl, (C₁-C₆)alkoxy, (C₁-C₆) haloalkyl, (C₁-C₆)haloalkoxy, —O—(C₂-C₆)alkenyl, —O—(C₂-C₆)alkynyl, (C₂-C₆)alkenyl, (C₂-C₆)alkynyl, —OH, —OP(O)(OH)₂, —OC(O)(C₁-C₆)alkyl, —C(O)(C₁-C₆)alkyl, —OC(O)O(C₁-C₆)alkyl, —NH₂, —NH((C₁-C₆)alkyl), —N((C₁-C₆)alkyl)₂, —NHC(O)(C₁-C₆)alkyl, —C(O)NH(C₁-C₆)alkyl, —S(O)₂(C₁-C₆)alkyl, —S(O)NH(C₁-C₆)alkyl, and —S(O)N((C₁-C₆)alkyl)₂. The substituents can themselves be optionally substituted. “Optionally substituted” as used herein also refers to substituted or unsubstituted whose meaning is described below.

As used herein, the term “substituted” means that the specified group or moiety bears one or more suitable substituents wherein the substituents may connect to the specified group or moiety at one or more positions. For example, an aryl substituted with a cycloalkyl may indicate that the cycloalkyl connects to one atom of the aryl with a bond or by fusing with the aryl and sharing two or more common atoms.

As used herein, the term “unsubstituted” means that the specified group bears no substituents.

Unless otherwise specifically defined, the term “aryl” refers to cyclic, aromatic hydrocarbon groups that have 1 to 3 aromatic rings, including monocyclic or bicyclic groups such as phenyl, biphenyl or naphthyl. Where containing two aromatic rings (bicyclic, etc.), the aromatic rings of the aryl group may be joined at a single point (e.g., biphenyl), or fused (e.g., naphthyl).

The aryl group may be optionally substituted by one or more substituents, e.g., 1 to 5 substituents, at any point of attachment. Exemplary substituents include, but are not limited to, —H, -halogen, —O—(C₁-C₆)alkyl, (C₁-C₆)alkyl, —O—(C₂-C₆)alkenyl, —O—(C₂-C₆)alkynyl, (C₂-C₆)alkenyl, (C₂-C₆)alkynyl, —OH, —OP(O)(OH)₂, —OC(O)(C₁-C₆)alkyl, —C(O)(C₁-C₆)alkyl, —OC(O)O(C₁-C₆)alkyl, —NH₂, —NH((C₁-C₆)alkyl), —N((C₁-C₆)alkyl)₂, —S(O)₂—(C₁-C₆)alkyl, —S(O)NH(C₁-C₆)alkyl, and —S(O)N((C₁-C₆)alkyl)₂. The substituents can themselves be optionally substituted.

Furthermore, when containing two fused rings the aryl groups herein defined may have one or more saturated or partially unsaturated ring fused with a fully unsaturated aromatic ring.

Exemplary ring systems of these aryl groups include, but are not limited to, phenyl, biphenyl, naphthyl, anthracenyl, phenalenyl, phenanthrenyl, indanyl, indenyl, tetrahydronaphthalenyl, tetrahydrobenzoannulenyl, and the like.

“Arenediyl”, “arylene” or “arylenyl” means an organic radical derived from an aromatic divalent radical by removal of at least two hydrogens, from a group such as phenyl, naphthyl, indenyl, indanyl and fluorenyl. An arylene is generally present as a bridging or linking group between two other groups. Non-exclusive examples of such arylene groups include 1,2-disubstituted phenyl, 1,3-disubstituted phenyl, 1,4-disubstituted phenyl, etc.

Unless otherwise specifically defined, “heteroaryl” means a monovalent monocyclic or a polycyclic aromatic radical of 5 to 24 ring atoms, containing one or more ring heteroatoms selected from N, O, S, P, or B, the remaining ring atoms being C. A polycyclic aromatic radical includes two or more fused rings and may further include two or more spiro-fused rings, e.g., bicyclic, tricyclic, tetracyclic, and the like. Unless otherwise specifically defined, “fused” means two rings sharing two ring atoms. Unless otherwise specifically defined, “spiro-fused” means two rings sharing one ring atom. Heteroaryl as herein defined also means a bicyclic heteroaromatic group wherein the heteroatom is selected from N, O, S, P, or B. Heteroaryl as herein defined also means a tricyclic heteroaromatic group containing one or more ring heteroatoms selected from N, O, S, P, or B. Heteroaryl as herein defined also means a tetracyclic heteroaromatic group containing one or more ring heteroatoms selected from N, O, S, P, or B. The aromatic radical is optionally substituted independently with one or more substituents described herein. Examples include, but are not limited to, furyl, thienyl, pyrrolyl, pyridyl, pyrazolyl, pyrimidinyl, imidazolyl, isoxazolyl, oxazolyl, oxadiazolyl, pyrazinyl, indolyl, thiophen-2-yl, quinolyl, benzopyranyl, isothiazolyl, thiazolyl, thiadiazole, indazole, benzimidazolyl, thieno[3,2-b]thiophene, triazolyl, triazinyl, imidazo[1,2-b]pyrazolyl, furo[2,3-c]pyridinyl, imidazo[1,2-a]pyridinyl, indazolyl, pyrrolo[2,3-c]pyridinyl, pyrrolo[3,2-c]pyridinyl, pyrazolo[3,4-c]pyridinyl, thieno[3,2-c]pyridinyl, thieno[2,3-c]pyridinyl, thieno[2,3-b]pyridinyl, benzothiazolyl, indolyl, indolinyl, indolinonyl, dihydrobenzothiophenyl, dihydrobenzofuranyl, benzofuran, chromanyl, thiochromanyl, tetrahydroquinolinyl, dihydrobenzothiazine, quinolinyl, isoquinolinyl, 1,6-naphthyridinyl, benzo[de]isoquinolinyl, pyrido[4,3-b][1,6]naphthyridinyl, thieno[2,3-b]pyrazinyl, quinazolinyl, tetrazolo[1,5-a]pyridinyl, [1,2,4]triazolo[4,3-a]pyridinyl, isoindolyl, pyrrolo[2,3-b]pyridinyl, pyrrolo[3,4-b]pyridinyl, pyrrolo[3,2-b]pyridinyl, imidazo[5,4-b]pyridinyl, pyrrolo[1,2-a]pyrimidinyl, tetrahydro pyrrolo[1,2-a]pyrimidinyl, 3,4-dihydro-2H-1-pyrrolo[2,1-b]pyrimidine, dibenzo[b,d]thiophene, pyridin-2-one, furo[3,2-c]pyridinyl, furo[2,3-c]pyridinyl, 1H-pyrido[3,4-b][1,4]thiazinyl, benzooxazolyl, benzoisoxazolyl, furo[2,3-b]pyridinyl, benzothiophenyl, 1,5-naphthyridinyl, furo[3,2-b]pyridine, [1,2,4]triazolo[1,5-a]pyridinyl, benzo[1,2,3]triazolyl, imidazo[1,2-a]pyrimidinyl, [1,2,4]triazolo[4,3-b]pyridazinyl, benzo[c][1,2,5]thiadiazolyl, benzo[c][1,2,5]oxadiazole, 1,3-dihydro-2H-benzo[d]imidazol-2-one, 3,4-dihydro-2H-pyrazolo[1,5-b][1,2]oxazinyl, 4,5,6,7-tetrahydropyrazolo[1,5-a]pyridinyl, thiazolo[5,4-d]thiazolyl, imidazo[2,1-b][1,3,4]thiadiazolyl, thieno[2,3-b]pyrrolyl, 3H-indolyl, and derivatives thereof.

Furthermore, when containing two or more fused rings, the heteroaryl groups defined herein may have one or more saturated or partially unsaturated ring fused with one or more fully unsaturated aromatic ring. In heteroaryl ring systems containing more than two fused rings, a saturated or partially unsaturated ring may further be fused with a saturated or partially unsaturated ring described herein. Furthermore, when containing three or more fused rings, the heteroaryl groups defined herein may have one or more saturated or partially unsaturated ring spiro-fused. Any saturated or partially unsaturated ring described herein is optionally substituted with one or more oxo. Exemplary ring systems of these heteroaryl groups include, for example, indolinyl, indolinonyl, dihydrobenzothiophenyl, dihydrobenzofuran, chromanyl, thiochromanyl, tetrahydroquinolinyl, dihydrobenzothiazine, 3,4-dihydro-1H-isoquinolinyl, 2,3-dihydrobenzofuranyl, benzofuranonyl, indolinyl, oxindolyl, indolyl, 1,6-dihydro-7H-pyrazolo[3,4-c]pyridin-7-onyl, 7,8-dihydro-6H-pyrido[3,2-b]pyrrolizinyl, 8H-pyrido[3,2-b]pyrrolizinyl, 1,5,6,7-tetrahydrocyclopenta[b]pyrazolo[4,3-e]pyridinyl, 7,8-dihydro-6H-pyrido[3,2-b]pyrrolizine, pyrazolo[1,5-a]pyrimidin-7(4H)-only, 3,4-dihydropyrazino[1,2-a]indol-1(2H)-onyl, benzo[c][1,2]oxaborol-1(3H)-olyl, 6,6a,7,8-tetrahydro-9H-pyrido[2,3-b]puyrrolo[1,2-d][1,4]oxazin-9-onyl, or 6a′,7′-dihydro-6′H,9′H-spiro[cyclopropane-1,8′-pyrido[2,3-b]pyrrolo[1,2-d][1,4]oxazin]-9′-onyl.

As used herein, the term “Heteroarendiyl” refers to divalent functional groups derived from heteroarenes by the removal of two hydrogen atoms from ring atoms.

“Halogen” or “halo” refers to fluorine, chlorine, bromine, or iodine.

“Alkyl” refers to a straight or branched chain saturated hydrocarbon containing 1-12 carbon atoms. Examples of a (C₁-C₆)alkyl group include, but are not limited to, methyl, ethyl, propyl, butyl, pentyl, hexyl, isopropyl, isobutyl, sec-butyl, tert-butyl, isopentyl, neopentyl, and isohexyl.

“Alkanediyl”, “alkylene” or “alkylenyl” refers to a straight or branched chain saturated hydrocarbon biradicals containing 1-12 carbon atoms. Unless specified otherwise, such alkanediyls include substituted alkanediyls. Typical alkylene groups include, but are not limited to, —CH₂—, —CH(CH₃)—, —C(CH₃)₂-, —CH₂CH₂—, —CH₂CH(CH₃)—, —CH₂C(CH₃)₂-, —CH₂CH₂CH₂—, —CH₂CH₂CH₂CH₂—, and the like.

“Alkoxy” refers to a straight or branched chain saturated hydrocarbon containing 1-12 carbon atoms containing a terminal “0” in the chain, i.e., —O(alkyl). Examples of alkoxy groups include without limitation, methoxy, ethoxy, propoxy, butoxy, tert-butoxy, or pentoxy groups.

“Alkenyl” refers to a straight or branched chain unsaturated hydrocarbon containing 2-12 carbon atoms. The “alkenyl” group contains at least one double bond in the chain. The double bond of an alkenyl group can be unconjugated or conjugated to another unsaturated group.

Examples of alkenyl groups include ethenyl, propenyl, n-butenyl, iso-butenyl, pentenyl, or hexenyl. An alkenyl group can be unsubstituted or substituted. Alkenyl, as herein defined, may be straight or branched.

“Alkenediyl” refers to a straight or branched chain alkene biradicals containing 2-12 carbon atoms. Unless specified otherwise, such alkenediyls include substituted alkenediyls. The “alkendiyl” group contains at least one double bond in the chain. The double bond of an alkendiyl group can be unconjugated or conjugated to another unsaturated group. Examples of alkenyl groups include ethendiyl, propendiyl, n-butendiyl, iso-butendiyl, pentendiyl, or hexendiyl.

“Alkynyl” refers to a straight or branched chain unsaturated hydrocarbon containing 2-12 carbon atoms. The “alkynyl” group contains at least one triple bond in the chain. Examples of alkenyl groups include ethynyl, propargyl, n-butynyl, iso-butynyl, pentynyl, or hexynyl. An alkynyl group can be unsubstituted or substituted.

“Alkynediyl” refers to a straight or branched chain unsaturated hydrocarbon biradicals containing 2-12 carbon atoms. The “alkynediyl” group contains at least one triple bond in the chain. Examples of alkendiyl groups include ethyndiyl, propynediyl, n-butynediyl, iso-butynediyl, pentynediyl, or hexynediyl. An alkynediyl group can be unsubstituted or substituted.

“Cycloalkyl” means mono or polycyclic saturated carbon rings containing 3-18 carbon atoms. Polycyclic cycloalkyl may be fused bicyclic cycloalkyl, bridged bicyclic cycloalkyl, or spiro-fused bicyclic cycloalkyl. A polycyclic cycloalkyl comprises at least one non-aromatic ring.

Examples of cycloalkyl groups include, without limitations, cyclopropyl, cyclobutyl, cyclopentyl, cyclohexyl, cycloheptanyl, cyclooctanyl, norbornyl, norborenyl, 1,2,3,4-tetrahydronaphthyl, 2,3-dihydro-TH-indenyl, spiro[3.5]nonyl, spiro [5.5]undecyl, bicyclo[1.1.1]pentanyl, bicyclo[2.2.2]octanyl, or bicyclo[2.2.2]octenyl.

“Cycloalkanediyl” refers to divalent functional groups derived from cycloalkanes by the removal of two hydrogen atoms from ring atoms. Cycloalkanediyl can be mono or polycyclic saturated carbon ring containing 3-18 carbon atoms. Polycyclic cycloalkanediyl may be fused bicyclic cycloalkyl, bridged bicyclic cycloalkyl, or spiro-fused bicyclic cycloalkanediyl. A polycyclic cycloalkanediyl comprises at least one non-aromatic ring.

“Heterocyclyl”, “heterocycle” or “heterocycloalkyl” mono or polycyclic rings containing 3-24 atoms which include carbon and one or more heteroatoms selected from N, O, S, P, or B and wherein the rings are not aromatic. The heterocycloalkyl ring structure may be substituted by one or more substituents. The substituents can themselves be optionally substituted. Examples of heterocyclyl rings include, but are not limited to, oxetanyl, azetidinyl, tetrahydrofuranyl, tetrahydropyranyl, pyrrolidinyl, oxazolinyl, oxazolidinyl, thiazolinyl, thiazolidinyl, pyranyl, thiopyranyl, tetrahydropyranyl, dioxalinyl, piperidinyl, morpholinyl, thiomorpholinyl, thiomorpholinyl S-oxide, thiomorpholinyl S-dioxide, piperazinyl, azepinyl, oxepinyl, diazepinyl, tropanyl, oxazolidinonyl, and homotropanyl.

As used herein, the term “Heterocyclediyl” refers to divalent functional groups derived from heterocycle by the removal of two hydrogen atoms from ring atoms.

The term “aromatic” means a planar ring having 4n+2 electrons in a conjugated system.

As used herein, “conjugated system” means a system of connected p-orbitals with delocalized electrons, and the system may include lone electron pairs.

The term “haloalkyl” as used herein refers to an alkyl group, as defined herein, which is substituted one or more halogen. Examples of haloalkyl groups include, but are not limited to, trifluoromethyl, difluoromethyl, pentafluoroethyl, trichloromethyl, etc.

The term “haloalkoxy” as used herein refers to an alkoxy group, as defined herein, which is substituted with one or more halogen. Examples of haloalkyl groups include, but are not limited to, trifluoromethoxy, difluoromethoxy, pentafluoroethoxy, trichloromethoxy, etc.

The term “cyano” as used herein means a substituent having a carbon atom joined to a nitrogen atom by a triple bond, i.e., CN.

The term “solvate” refers to a complex of variable stoichiometry formed by a solute and solvent. Such solvents for the purpose of the disclosure may not interfere with the biological activity of the solute. Examples of suitable solvents include, but are not limited to, water, MeOH, EtOH, and AcOH. Solvates wherein water is the solvent molecule are typically referred to as hydrates. Hydrates include compositions containing stoichiometric amounts of water, as well as compositions containing variable amounts of water.

The term “isomer” refers to compounds that have the same composition and molecular weight but differ in physical and/or chemical properties. The structural difference may be in constitution (geometric isomers) or in the ability to rotate the plane of polarized light (stereoisomers). With regard to stereoisomers, the compounds of Formula (A) may have one or more asymmetric carbon atom and may occur as racemates, racemic mixtures and as individual enantiomers or diastereomers.

The term “stereoisomer” refers to compounds that have the same molecular formula and sequence of bonded atoms (constitution) but differ in the three-dimensional orientations of their atoms in space.

The present disclosure also contemplates isotopically-labelled compounds of Formula I (e.g., those labeled with ²H and ¹⁴C). Deuterated (i.e., ²H or D) and carbon-14 (i.e., ¹⁴C) isotopes are particularly preferred for their ease of preparation and detectability. Further, substitution with heavier isotopes such as deuterium may afford certain therapeutic advantages resulting from greater metabolic stability (e.g., increased in vivo half-life or reduced dosage requirements) and hence may be preferred in some circumstances. Isotopically labelled compounds of Formula I can generally be prepared by following procedures analogous to those disclosed in the Schemes and/or in the Examples herein below, by substituting an appropriate isotopically labelled reagent for a non-isotopically labelled reagent.

The disclosure also includes pharmaceutical compositions comprising a therapeutically effective amount of a disclosed compound and a pharmaceutically acceptable carrier.

Representative “pharmaceutically acceptable salts” include, e.g., water-soluble and water-insoluble salts, such as the acetate, amsonate (4,4-diaminostilbene-2,2-disulfonate), benzenesulfonate, benzoate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fumarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, magnesium, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate, pantothenate, phosphate/diphosphate, picrate, polygalacturonate, propionate, p-toluenesulfonate, salicylate, stearate, subacetate, succinate, sulfate, sulfosalicylate, tannate, tartrate, teoclate, tosylate, triethiodide, and valerate salts.

A “patient” or “subject” is amammal, e.g., ahuman, mouse, rat, guinea pig, dog, cat, horse, cow, pig, or non-human primate, such as a monkey, chimpanzee, baboon, or rhesus.

An “effective amount” when used in connection with a compound is an amount effective for treating or preventing a disease in a subject as described herein.

The term “carrier”, as used in this disclosure, encompasses carriers, excipients, and diluents and means a material, composition or vehicle, such as a liquid or solid filler, diluent, excipient, solvent or encapsulating material, involved in carrying or transporting a pharmaceutical agent from one organ, or portion of the body, to another organ, or portion of the body of a subject.

The term “treating” with regard to a subject, refers to improving at least one symptom of the subject's disorder. Treating includes curing, improving, or at least partially ameliorating the disorder.

The term “disorder” is used in this disclosure to mean, and is used interchangeably with, the terms disease, condition, or illness, unless otherwise indicated.

The term “administer”, “administering”, or “administration” as used in this disclosure refers to either directly administering a disclosed compound or pharmaceutically acceptable salt of the disclosed compound or a composition to a subject, or administering a prodrug derivative or analog of the compound or pharmaceutically acceptable salt of the compound or composition to the subject, which can form an equivalent amount of active compound within the subject's body.

The term “prodrug,” as used in this disclosure, means a compound which is convertible in vivo by metabolic means (e.g., by hydrolysis) to a disclosed compound.

The term “salt” refers to pharmaceutically acceptable salts.

The term “pharmaceutically acceptable salt” also refers to a salt of the compositions of the present disclosure having an acidic functional group, such as a carboxylic acid functional group, and a base.

The symbol “ [symbol] ”, as used in this disclosure, means a double bond in E- or Z-configuration or mixture of E- and Z-configurations.

“EGFR inhibitor” as used herein refer to compounds of Formula I and/or compositions comprising a compound of Formula I which inhibits EGFR.

The amount of compound of composition described herein needed for achieving a therapeutic effect may be determined empirically in accordance with conventional procedures for the particular purpose. Generally, for administering therapeutic agents (e.g. compounds or compositions of Formula I (and/or additional agents) described herein) for therapeutic purposes, the therapeutic agents are given at a pharmacologically effective dose. A “pharmacologically effective amount,” “pharmacologically effective dose,” “therapeutically effective amount,” or “effective amount” refers to an amount sufficient to produce the desired physiological effect or amount capable of achieving the desired result, particularly for treating the disorder or disease. An effective amount as used herein would include an amount sufficient to, for example, delay the development of a symptom of the disorder or disease, alter the course of a symptom of the disorder or disease (e.g., slow the progression of a symptom of the disease), reduce or eliminate one or more symptoms or manifestations of the disorder or disease, and reverse a symptom of a disorder or disease. For example, administration of therapeutic agents to a subject suffering from cancer provides a therapeutic benefit not only when the underlying condition is eradicated or ameliorated, but also when the subject reports a decrease in the severity or duration of the symptoms associated with the disease, e.g., a decrease in tumor burden, a decrease in circulating tumor cells, an increase in progression free survival. Therapeutic benefit also includes halting or slowing the progression of the underlying disease or disorder, regardless of whether improvement is realized.

### Compounds of the Present Disclosure

In one aspect, the present disclosure provides compounds of Formula (A) and salts, stereoisomers, solvates, prodrugs, isotopic derivatives, and tautomers thereof.

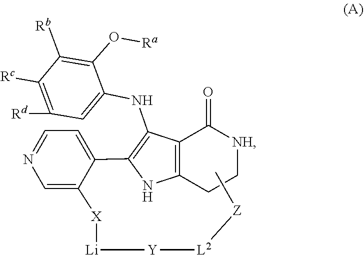

Wherein R^{a}, R^{b}, R^{c}, R^{d}, X, L¹, Y, L², and Z are as described herein.

It is understood that, for a compound of Formula (A), R^{a}, R^{b}, R^{c}, R^{d}, X, L¹, Y, L², and Z can each be, where applicable, selected from the groups described herein, and any group described herein for any R^{a}, R^{b}, R^{c}, R^{d}, X, L¹, Y, L², and Z can be combined, where applicable, with any group described herein for one or more of the remainder of R^{a}, R^{b}, R^{c}, R^{d}, X, L¹, Y, L², and Z.

In some embodiments, the compound is of Formula (A)

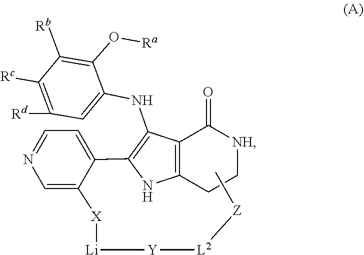

or a pharmaceutically acceptable salt, prodrug, stereoisomer, solvate, or tautomer thereof, wherein
R^{a} is selected from H, C₁-C₆ alkyl, C₂-C₆ alkenyl, C₂-C₆ alkynyl, C₃-C₁₀ cycloalkyl, heterocyclyl, aryl, and heteroaryl, wherein the alkyl, alkenyl, alkynyl, cycloalkyl, heterocyclyl, aryl, or heteroaryl is optionally substituted with one or more substituents independently selected from halogen, OH, ═O, NR³R⁴, CN, NO₂, COOR¹, CONR³R⁴;
R^{b}, R^{c}, R^{d} are each independently selected from H, halogen, OH, NH₂, CN, NO₂, C₁-C₆ alkyl, and C₁-C₆ alkoxy, wherein alkyl or alkoxy is optionally substituted with one or more halogen, CN;
X is selected from a bond, O, NR¹, C(R²)₂, S, S(O), S(O)₂, C(O), C(O)O, C(O)NR¹, OC(O)O, N(R²)C(O)NR², arenediyl;
R¹ is selected from H, C₁-C₆ alkyl, C₃-C₁₀ cycloalkyl, C₂-C₆ alkenyl, C₂-C₆ alkynyl, C₁-C₆ acyl, heterocycle, aryl, heteroaryl, and S(O)₂R² wherein the alkyl, alkenyl, alkynyl, heterocycle, cycloalkyl, aryl, or heteroaryl is optionally substituted with one or more substituents independently selected from halogen, OH, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, heteroaryl;
each R² is independently selected from H, C₁-C₆ alkyl, C₃-C₁₀ cycloalkyl, C₂-C₆ alkenyl, C₂-C₆ alkynyl, heterocycle, aryl, and heteroaryl wherein the alkyl, alkenyl, alkynyl, heterocycle, cycloalkyl, aryl, or heteroaryl is optionally substituted with one or more substituents independently selected from halogen, OH, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, heteroaryl;
Y is selected from a bond, O, NR¹, C(R²)₂, S, S(O), S(O)₂, C(O), C(O)O, C(O)NR¹, OC(O)O, N(R²)C(O)NR²;
Z is selected from a bond, O, C(R²)₂;
L¹ is selected from heterocyclediyl-C₁-C₁₀-alkanediyl, C₁-C₁₀ alkanediyl, C₂-C₁₀ alkenediyl, C₂-C₁₀ alkynediyl, C₃-C₁₀-cycloalkanediyl, C₃-C₁₀-cycloalkanediyl-C₁-C₁₀-alkanediyl, arenediyl, arenediyl-C₁-C₁₀-alkanediyl, heterocyclediyl, heterocyclediyl-C(O), heteroarenediyl, heteroarenediyl-C₁-C₁₀ alkanediyl, C₁-C₆ alkanediyl-O—C₁-C₆ alkanediyl, C₁-C₆ alkanediyl-NR¹—C₁-C₆ alkanediyl, C₁-C₆ alkanediyl-C(O)NR¹—C₁-C₆ alkanediyl, wherein alkanediyl, alkendiyl, alkynediyl, arenediyl, heterocyclediyl or heteroarenediyl heteroaryl is optionally substituted with one or more substituents independently selected from halogen, OH, oxo, CN, C₁-C₆ alkyl, C₁-C₆ alkoxy, C₁-C₆ alkyl-C₁-C₆ alkoxy, C₁-C₆ alkyl-NR¹—C₁-C₆ alkyl, NR³R⁴, —C(O)NR³R⁴, —S(O)C₁-C₆ alkyl, —S(O)₂NR³R⁴, aryl, and —S(O)₂C₁-C₆ alkyl;
L² is selected from C₁-C₁₀ alkanediyl, C₂-C₁₀ alkenediyl, C₂-C₁₀ alkynediyl, C₃-C₁₀-cycloalkanediyl, C₃-C₁₀-cycloalkanediyl-C₁-C₁₀-alkanediyl, arenediyl, arenediyl-C₁-C₁₀-alkanediyl, heterocyclediyl, heterocyclediyl-C₁-C₁₀-alkanediyl, heterocyclediyl-C(O), heteroarenediyl, heteroarenediyl-C₁-C₁₀ alkanediyl, C₁-C₆ alkanediyl-O—C₁-C₆ alkanediyl, C₁-C₆ alkanediyl-NR¹—C₁-C₆ alkanediyl, C₁-C₆ alkanediyl-C(O)NR¹—C₁-C₆ alkanediyl, wherein alkanediyl, alkendiyl, alkynediyl, arenediyl, heterocyclediyl or heteroarenediyl heteroaryl is optionally substituted with one or more substituents independently selected from halogen, OH, oxo, CN, C₁-C₆ alkyl, C₁-C₆ alkoxy, C₁-C₆ alkyl-C₁-C₆ alkoxy, C₁-C₆ alkyl-NR¹—C₁-C₆ alkyl, NR³R⁴, —C(O)NR³R⁴, —S(O)C₁-C₆ alkyl, —S(O)₂NR³R⁴, aryl, and —S(O)₂C₁-C₆ alkyl;
R³ and R⁴ are each independently selected from H, C₁-C₆ alkyl, C₁-C₆ acyl and C₃-C₁₀ cycloalkyl;
or R³ and R⁴ together with the atoms to which they are attached and any intervening atoms, form a 5-14 membered heterocycle, wherein the heterocycle is optionally substituted with one or more substituents independently selected from halogen, OH, ═O, CN, NO₂, C₁-C₆ alkyl, C₁-C₆ alkoxy.

In some embodiments, the compound is of Formula (A-1):

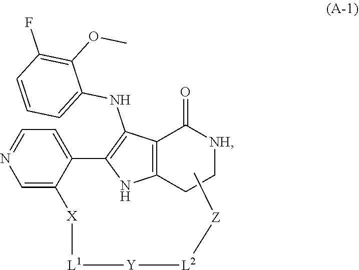

or a pharmaceutically acceptable salt, prodrug, stereoisomer, solvate, or tautomer thereof.

In some embodiments, the compound is of Formula (A-1-a):

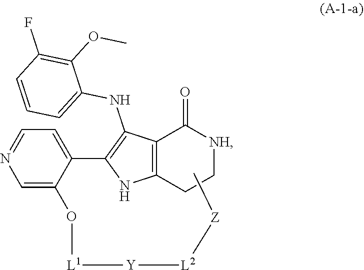

or a pharmaceutically acceptable salt, prodrug, stereoisomer, solvate, or tautomer thereof.

In some embodiments, the compound is of Formula (A-1-b):

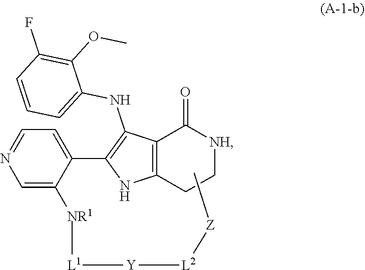

or a pharmaceutically acceptable salt, prodrug, stereoisomer, solvate, or tautomer thereof.

In some embodiments, the compound is of Formula (A-1-b-H):

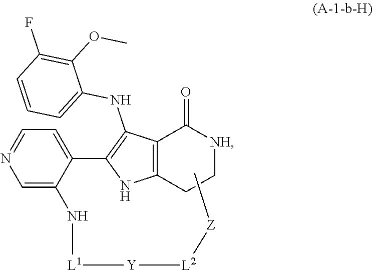

or a pharmaceutically acceptable salt, prodrug, stereoisomer, solvate, or tautomer thereof.

In some embodiments, the compound is of Formula (A-A):

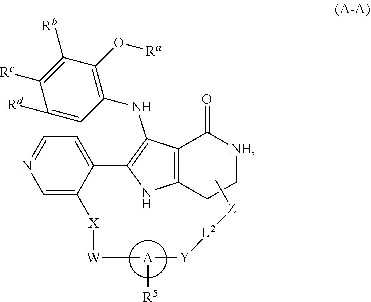

or a pharmaceutically acceptable salt, prodrug, stereoisomer, solvate, or tautomer thereof, wherein Ring A is selected from C₃-C₁₀-cycloalkane, arene, heterocycle, heteroarene; W is (CH₂)_{w}; w is an integer selected from 0, 1, 2, 3, 4, 5, 6, 7, 8, 9, and 10; R⁵ is selected from H, halogen, OH, oxo, CN, C₁-C₆ alkyl, C₁-C₆ alkoxy, C₁-C₆ alkyl-C₁-C₆ alkoxy, C₁-C₆ alkyl-NR¹—C₁-C₆ alkyl, NR³R⁴, —C(O)NR³R⁴, —S(O)C₁-C₆ alkyl, —S(O)₂NR³R⁴, and —S(O)₂C₁-C₆ alkyl and all other variables are as defined herein.

In some embodiments, the compound is of Formula (A-A-A):

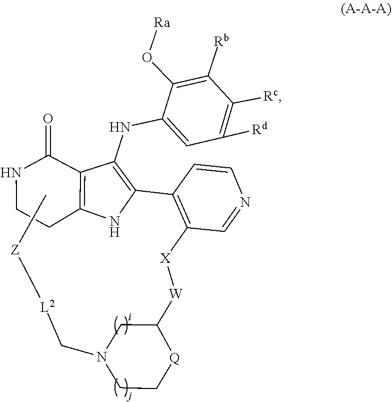

or a pharmaceutically acceptable salt, prodrug, stereoisomer, solvate, or tautomer thereof, wherein i is an integer selected from 0, 1, 2; j is an integer selected from 0, 1, 2; W is (CH₂)_{w}; w is an integer selected from 0, 1, 2, 3, 4, 5, 6, 7, 8, 9, and 10; Q is selected from a bond, CH₂, NH, O, S, S(O), S(O)₂ and all other variables are as defined herein.

In some embodiments, the compound is of Formula (A-A-A-I):

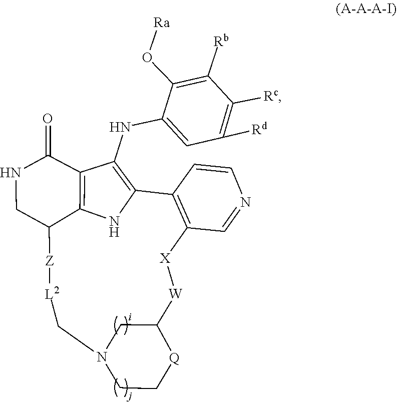

or a pharmaceutically acceptable salt, prodrug, stereoisomer, solvate, or tautomer thereof, wherein i is an integer selected from 0, 1, 2; j is an integer selected from 0, 1, 2; W is (CH₂)_{w}; w is an integer selected from 0, 1, 2, 3, 4, 5, 6, 7, 8, 9, and 10; Q is selected from a bond, CH₂, NH, O, S, S(O), S(O)₂ and all other variables are as defined herein.

In some embodiments, the compound is of Formula (A-A-A-II):

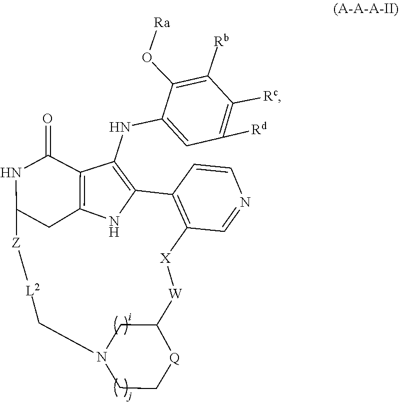

or a pharmaceutically acceptable salt, prodrug, stereoisomer, solvate, or tautomer thereof, wherein i is an integer selected from 0, 1, 2; j is an integer selected from 0, 1, 2; W is (CH₂)_{w}; w is an integer selected from 0, 1, 2, 3, 4, 5, 6, 7, 8, 9, and 10; Q is selected from a bond, CH₂, NH, O, S, S(O), S(O)₂ and all other variables are as defined herein.

In some embodiments, the compound is of Formula (A-A-A-1):

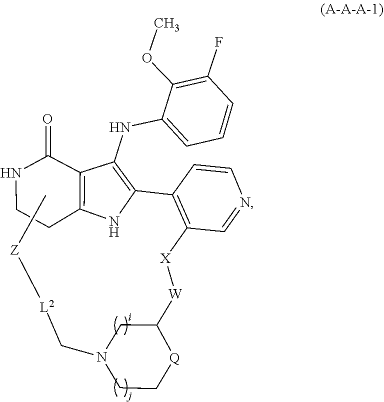

or a pharmaceutically acceptable salt, prodrug, stereoisomer, solvate, or tautomer thereof, wherein i is an integer selected from 0, 1, 2; j is an integer selected from 0, 1, 2; W is (CH₂)_{w}; w is an integer selected from 0, 1, 2, 3, 4, 5, 6, 7, 8, 9, and 10; Q is selected from a bond, CH₂, NH, O, S, S(O), S(O)₂ and all other variables are as defined herein.

In some embodiments, the compound is of Formula (A-A-A-1-I):

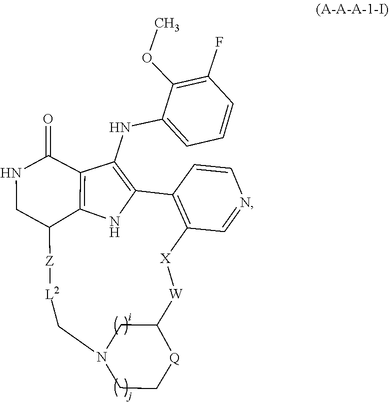

or a pharmaceutically acceptable salt, prodrug, stereoisomer, solvate, or tautomer thereof, wherein i is an integer selected from 0, 1, 2; j is an integer selected from 0, 1, 2; W is (CH₂)_{w}; w is an integer selected from 0, 1, 2, 3, 4, 5, 6, 7, 8, 9, and 10; Q is selected from a bond, CH₂, NH, O, S, S(O), S(O)₂ and all other variables are as defined herein.

In some embodiments, the compound is of Formula (A-A-A-1-II):

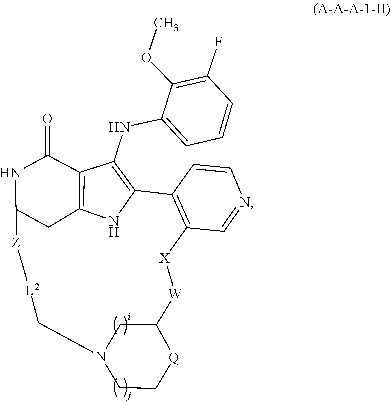

or a pharmaceutically acceptable salt, prodrug, stereoisomer, solvate, or tautomer thereof, wherein i is an integer selected from 0, 1, 2; j is an integer selected from 0, 1, 2; W is (CH₂)_{w}; w is an integer selected from 0, 1, 2, 3, 4, 5, 6, 7, 8, 9, and 10; Q is selected from a bond, CH₂, NH, O, S, S(O), S(O)₂ and all other variables are as defined herein.

In some embodiments, the compound is of Formula (A-1-a-I):

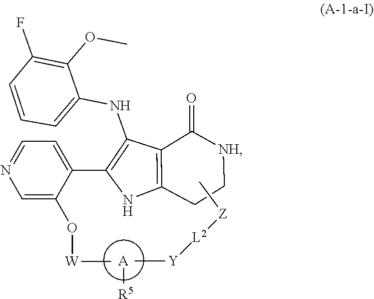

or a pharmaceutically acceptable salt, prodrug, stereoisomer, solvate, or tautomer thereof, wherein Ring A is selected from C₃-C₁₀-cycloalkane, arene, heterocycle, heteroarene; W is (CH₂)_{w}; w is an integer selected from 0, 1, 2, 3, 4, 5, 6, 7, 8, 9, and 10; R⁵ is selected from H, halogen, OH, oxo, CN, C₁-C₆ alkyl, C₁-C₆ alkoxy, C₁-C₆ alkyl-C₁-C₆ alkoxy, C₁-C₆ alkyl-NR¹—C₁-C₆ alkyl, NR³R⁴, —C(O)NR³R⁴, —S(O)C₁-C₆ alkyl, —S(O)₂NR³R⁴, and —S(O)₂C₁-C₆ alkyl and all other variables are as defined herein.

In some embodiments, the compound is of Formula (A-1-a-I-A):

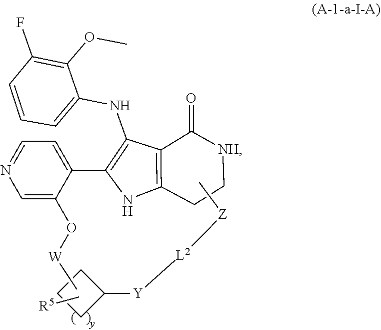

or a pharmaceutically acceptable salt, prodrug, stereoisomer, solvate, or tautomer thereof, wherein W is (CH₂)_{w}; w is an integer selected from 0, 1, 2, 3, 4, 5, 6, 7, 8, 9, and 10; v is an integer selected from 0, 1, 2, 3, 4 and 5; R⁵ is selected from H, halogen, OH, oxo, CN, C₁-C₆ alkyl, C₁-C₆ alkoxy, C₁-C₆ alkyl-C₁-C₆ alkoxy, C₁-C₆ alkyl-NR¹—C₁-C₆ alkyl, NR³R⁴, —C(O)NR³R⁴, —S(O)C₁-C₆ alkyl, —S(O)₂NR³R⁴, and —S(O)₂C₁-C₆ alkyl; and all other variables are as defined herein.

In some embodiments, the compound is of Formula (A-1-a-I-A-1):

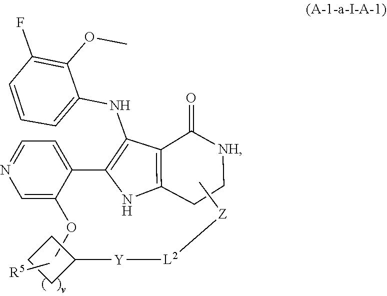

or a pharmaceutically acceptable salt, prodrug, stereoisomer, solvate, or tautomer thereof, wherein v is an integer selected from 0, 1, 2, 3, 4 and 5; R⁵ is selected from H, halogen, OH, oxo, CN, C₁-C₆ alkyl, C₁-C₆ alkoxy, C₁-C₆ alkyl-C₁-C₆ alkoxy, C₁-C₆ alkyl-NR¹—C₁-C₆ alkyl, NR³R⁴, —C(O)NR³R⁴, —S(O)C₁-C₆ alkyl, —S(O)₂NR³R⁴, and —S(O)₂C₁-C₆ alkyl; and all other variables are as defined herein.

In some embodiments, the compound is of Formula (A-1-a-I-B):

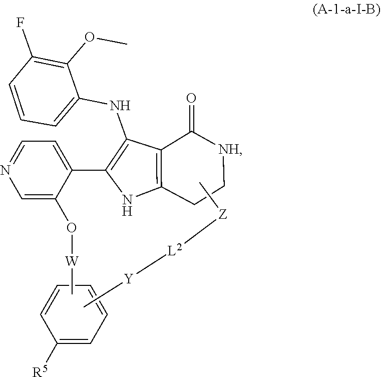

or a pharmaceutically acceptable salt, prodrug, stereoisomer, solvate, or tautomer thereof, wherein W is (CH₂)_{w}; w is an integer selected from 0, 1, 2, 3, 4, 5, 6, 7, 8, 9, and 10; R⁵ is selected from H, halogen, OH, oxo, CN, C₁-C₆ alkyl, C₁-C₆ alkoxy, C₁-C₆ alkyl-C₁-C₆ alkoxy, C₁-C₆ alkyl-NR¹—C₁-C₆ alkyl, NR³R⁴, —C(O)NR³R⁴, —S(O)C₁-C₆ alkyl, —S(O)₂NR³R⁴, and —S(O)₂C₁-C₆ alkyl and all other variables are as defined herein.

In some embodiments, the compound is of Formula (A-1-a-I-B-0):

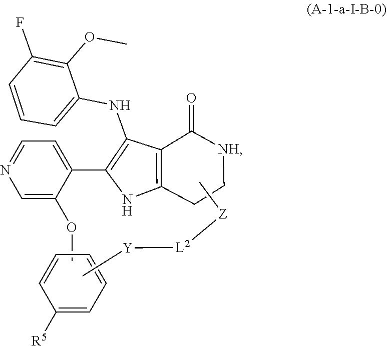

or a pharmaceutically acceptable salt, prodrug, stereoisomer, solvate, or tautomer thereof, wherein R⁵ is selected from H, halogen, OH, oxo, CN, C₁-C₆ alkyl, C₁-C₆ alkoxy, C₁-C₆ alkyl-C₁-C₆ alkoxy, C₁-C₆ alkyl-NR¹—C₁-C₆ alkyl, NR³R⁴, —C(O)NR³R⁴, —S(O)C₁-C₆ alkyl, —S(O)₂NR³R⁴, and —S(O)₂C₁-C₆ alkyl and all other variables are as defined herein.

In some embodiments, the compound is of Formula (A-1-a-I-B-1):

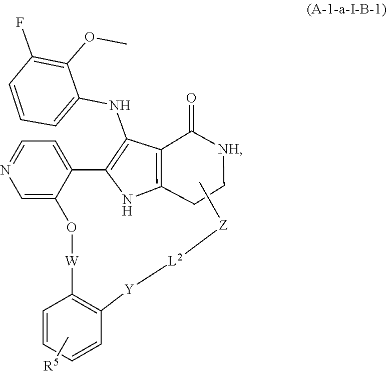

or a pharmaceutically acceptable salt, prodrug, stereoisomer, solvate, or tautomer thereof, wherein W is (CH₂)_{w}; w is an integer selected from 0, 1, 2, 3, 4, 5, 6, 7, 8, 9, and 10; R⁵ is selected from H, halogen, OH, oxo, CN, C₁-C₆ alkyl, C₁-C₆ alkoxy, C₁-C₆ alkyl-C₁-C₆ alkoxy, C₁-C₆ alkyl-NR¹—C₁-C₆ alkyl, NR³R⁴, —C(O)NR³R⁴, —S(O)C₁-C₆ alkyl, —S(O)₂NR³R⁴, and —S(O)₂C₁-C₆ alkyl and all other variables are as defined herein.

In some embodiments, the compound is of Formula (A-1-a-I-B-2):

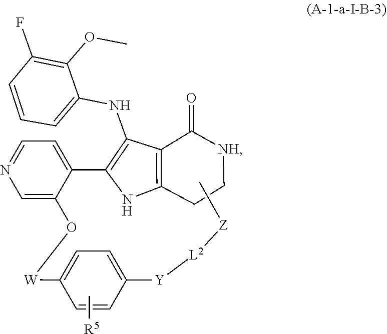

or a pharmaceutically acceptable salt, prodrug, stereoisomer, solvate, or tautomer thereof, wherein W is (CH₂)_{w}; w is an integer selected from 0, 1, 2, 3, 4, 5, 6, 7, 8, 9, and 10; R⁵ is selected from H, halogen, OH, oxo, CN, C₁-C₆ alkyl, C₁-C₆ alkoxy, C₁-C₆ alkyl-C₁-C₆ alkoxy, C₁-C₆ alkyl-NR¹—C₁-C₆ alkyl, NR³R⁴, —C(O)NR³R⁴, —S(O)C₁-C₆ alkyl, —S(O)₂NR³R⁴, and —S(O)₂C₁-C₆ alkyl and all other variables are as defined herein.

In some embodiments, the compound is of Formula (A-1-a-I-B-3):

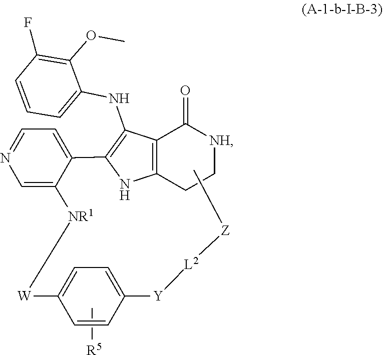

or a pharmaceutically acceptable salt, prodrug, stereoisomer, solvate, or tautomer thereof, wherein W is (CH₂)_{w}; w is an integer selected from 0, 1, 2, 3, 4, 5, 6, 7, 8, 9, and 10; R⁵ is selected from H, halogen, OH, oxo, CN, C₁-C₆ alkyl, C₁-C₆ alkoxy, C₁-C₆ alkyl-C₁-C₆ alkoxy, C₁-C₆ alkyl-NR¹—C₁-C₆ alkyl, NR³R⁴, —C(O)NR³R⁴, —S(O)C₁-C₆ alkyl, —S(O)₂NR³R⁴, and —S(O)₂C₁-C₆ alkyl and all other variables are as defined herein.

In some embodiments, the compound is of Formula (A-1-a-I-C):

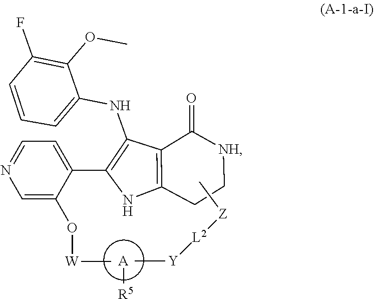

or a pharmaceutically acceptable salt, prodrug, stereoisomer, solvate, or tautomer thereof, wherein Ring H is 3-10 membered monocyclic or bicyclic heterocycle comprising 1-3 heteroatoms selected from N, O, S; W is (CH₂)_{w}; w is an integer selected from 0, 1, 2, 3, 4, 5, 6, 7, 8, 9, and 10; R⁵ is selected from H, halogen, OH, oxo, CN, C₁-C₆ alkyl, C₁-C₆ alkoxy, C₁-C₆ alkyl-C₁-C₆ alkoxy, C₁-C₆ alkyl-NR¹—C₁-C₆ alkyl, NR³R⁴, —C(O)NR³R⁴, —S(O)C₁-C₆ alkyl, —S(O)₂NR³R⁴, and —S(O)₂C₁-C₆ alkyl and all other variables are as defined herein.

In some embodiments, the compound is of Formula (A-1-a-I-C-1):

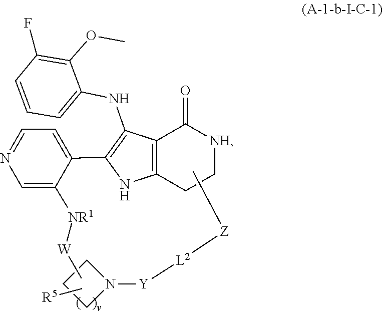

or a pharmaceutically acceptable salt, prodrug, stereoisomer, solvate, or tautomer thereof, wherein W is (CH₂)_{w}; w is an integer selected from 0, 1, 2, 3, 4, 5, 6, 7, 8, 9, and 10; v is an integer selected from 0, 1, 2, 3, 4 and 5; R⁵ is selected from H, halogen, OH, oxo, CN, C₁-C₆ alkyl, C₁-C₆ alkoxy, C₁-C₆ alkyl-C₁-C₆ alkoxy, C₁-C₆ alkyl-NR¹—C₁-C₆ alkyl, NR³R⁴, —C(O)NR³R⁴, —S(O)C₁-C₆ alkyl, —S(O)₂NR³R⁴, and —S(O)₂C₁-C₆ alkyl; and all other variables are as defined herein.

In some embodiments, the compound is of Formula (A-1-a-I-C-1-a):

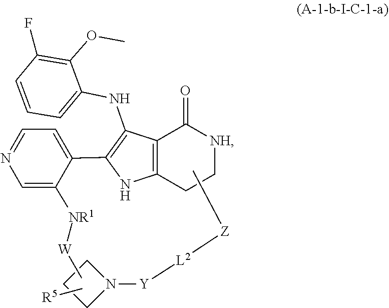

or a pharmaceutically acceptable salt, prodrug, stereoisomer, solvate, or tautomer thereof, wherein W is (CH₂)_{w}; w is an integer selected from 0, 1, 2, 3, 4, 5, 6, 7, 8, 9, and 10; R⁵ is selected from H, halogen, OH, oxo, CN, C₁-C₆ alkyl, C₁-C₆ alkoxy, C₁-C₆ alkyl-C₁-C₆ alkoxy, C₁-C₆ alkyl-NR¹—C₁-C₆ alkyl, NR³R⁴, —C(O)NR³R⁴, —S(O)C₁-C₆ alkyl, —S(O)₂NR³R⁴, and —S(O)₂C₁-C₆ alkyl; and all other variables are as defined herein.

In some embodiments, the compound is of Formula (A-1-a-I-C-1-a-I):

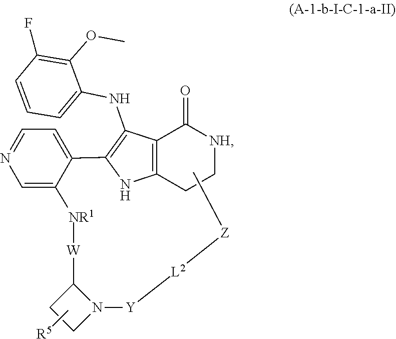

or a pharmaceutically acceptable salt, prodrug, stereoisomer, solvate, or tautomer thereof, wherein W is (CH₂)_{w}; w is an integer selected from 0, 1, 2, 3, 4, 5, 6, 7, 8, 9, and 10; R⁵ is selected from H, halogen, OH, oxo, CN, C₁-C₆ alkyl, C₁-C₆ alkoxy, C₁-C₆ alkyl-C₁-C₆ alkoxy, C₁-C₆ alkyl-NR¹—C₁-C₆ alkyl, NR³R⁴, —C(O)NR³R⁴, —S(O)C₁-C₆ alkyl, —S(O)₂NR³R⁴, and —S(O)₂C₁-C₆ alkyl; and all other variables are as defined herein.

In some embodiments, the compound is of Formula (A-1-a-I-C-1-a-II):

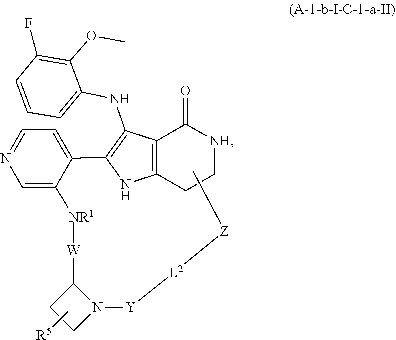

or a pharmaceutically acceptable salt, prodrug, stereoisomer, solvate, or tautomer thereof, wherein W is (CH₂)_{w}; w is an integer selected from 0, 1, 2, 3, 4, 5, 6, 7, 8, 9, and 10; R⁵ is selected from H, halogen, OH, oxo, CN, C₁-C₆ alkyl, C₁-C₆ alkoxy, C₁-C₆ alkyl-C₁-C₆ alkoxy, C₁-C₆ alkyl-NR¹—C₁-C₆ alkyl, NR³R⁴, —C(O)NR³R⁴, —S(O)C₁-C₆ alkyl, —S(O)₂NR³R⁴, and —S(O)₂C₁-C₆ alkyl; and all other variables are as defined herein.

In some embodiments, the compound is of Formula (A-1-a-I-C-1-b):

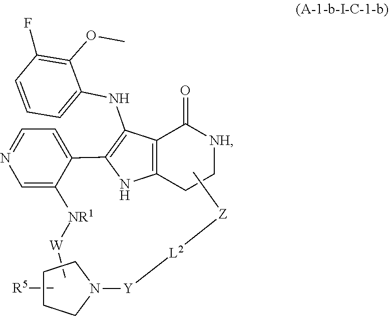

or a pharmaceutically acceptable salt, prodrug, stereoisomer, solvate, or tautomer thereof, wherein W is (CH₂)_{w}; w is an integer selected from 0, 1, 2, 3, 4, 5, 6, 7, 8, 9, and 10; R⁵ is selected from H, halogen, OH, oxo, CN, C₁-C₆ alkyl, C₁-C₆ alkoxy, C₁-C₆ alkyl-C₁-C₆ alkoxy, C₁-C₆ alkyl-NR¹—C₁-C₆ alkyl, NR³R⁴, —C(O)NR³R⁴, —S(O)C₁-C₆ alkyl, —S(O)₂NR³R⁴, and —S(O)₂C₁-C₆ alkyl; and all other variables are as defined herein.

In some embodiments, the compound is of Formula (A-1-a-I-C-1-b-I):

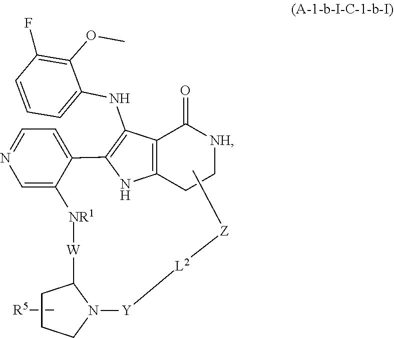

or a pharmaceutically acceptable salt, prodrug, stereoisomer, solvate, or tautomer thereof, wherein W is (CH₂)_{w}; w is an integer selected from 0, 1, 2, 3, 4, 5, 6, 7, 8, 9, and 10; R⁵ is selected from H, halogen, OH, oxo, CN, C₁-C₆ alkyl, C₁-C₆ alkoxy, C₁-C₆ alkyl-C₁-C₆ alkoxy, C₁-C₆ alkyl-NR¹—C₁-C₆ alkyl, NR³R⁴, —C(O)NR³R⁴, —S(O)C₁-C₆ alkyl, —S(O)₂NR³R⁴, and —S(O)₂C₁-C₆ alkyl; and all other variables are as defined herein.

In some embodiments, the compound is of Formula (A-1-a-I-C-1-b-II):

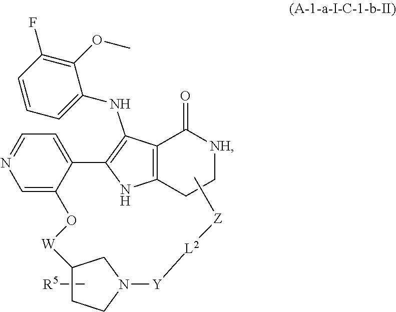

or a pharmaceutically acceptable salt, prodrug, stereoisomer, solvate, or tautomer thereof, wherein W is (CH₂)_{w}; w is an integer selected from 0, 1, 2, 3, 4, 5, 6, 7, 8, 9, and 10; R⁵ is selected from H, halogen, OH, oxo, CN, C₁-C₆ alkyl, C₁-C₆ alkoxy, C₁-C₆ alkyl-C₁-C₆ alkoxy, C₁-C₆ alkyl-NR¹—C₁-C₆ alkyl, NR³R⁴, —C(O)NR³R⁴, —S(O)C₁-C₆ alkyl, —S(O)₂NR³R⁴, and —S(O)₂C₁-C₆ alkyl; and all other variables are as defined herein.

In some embodiments, the compound is of Formula (A-1-a-I-C-1-c):

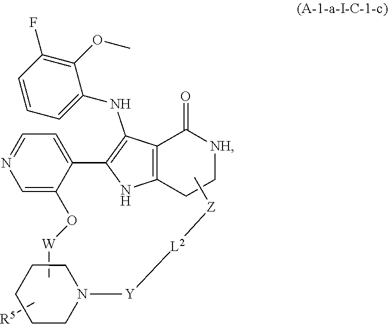

or a pharmaceutically acceptable salt, prodrug, stereoisomer, solvate, or tautomer thereof, wherein W is (CH₂)_{w}; w is an integer selected from 0, 1, 2, 3, 4, 5, 6, 7, 8, 9, and 10; R⁵ is selected from H, halogen, OH, oxo, CN, C₁-C₆ alkyl, C₁-C₆ alkoxy, C₁-C₆ alkyl-C₁-C₆ alkoxy, C₁-C₆ alkyl-NR¹—C₁-C₆ alkyl, NR³R⁴, —C(O)NR³R⁴, —S(O)C₁-C₆ alkyl, —S(O)₂NR³R⁴, and —S(O)₂C₁-C₆ alkyl; and all other variables are as defined herein.

In some embodiments, the compound is of Formula (A-1-a-I-C-1-c-I):

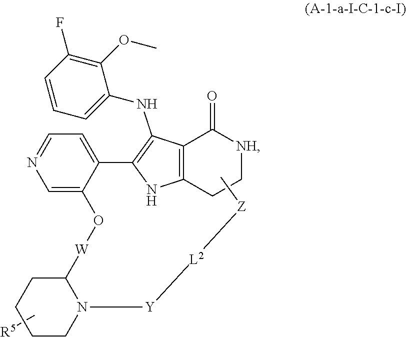

or a pharmaceutically acceptable salt, prodrug, stereoisomer, solvate, or tautomer thereof, wherein W is (CH₂)_{w}; w is an integer selected from 0, 1, 2, 3, 4, 5, 6, 7, 8, 9, and 10; R⁵ is selected from H, halogen, OH, oxo, CN, C₁-C₆ alkyl, C₁-C₆ alkoxy, C₁-C₆ alkyl-C₁-C₆ alkoxy, C₁-C₆ alkyl-NR¹—C₁-C₆ alkyl, NR³R⁴, —C(O)NR³R⁴, —S(O)C₁-C₆ alkyl, —S(O)₂NR³R⁴, and —S(O)₂C₁-C₆ alkyl; and all other variables are as defined herein.

In some embodiments, the compound is of Formula (A-1-a-I-C-1-c-II):

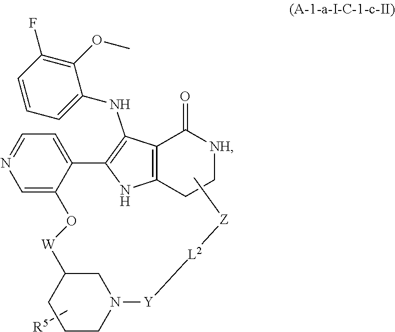

or a pharmaceutically acceptable salt, prodrug, stereoisomer, solvate, or tautomer thereof, wherein W is (CH₂)_{w}; w is an integer selected from 0, 1, 2, 3, 4, 5, 6, 7, 8, 9, and 10; R⁵ is selected from H, halogen, OH, oxo, CN, C₁-C₆ alkyl, C₁-C₆ alkoxy, C₁-C₆ alkyl-C₁-C₆ alkoxy, C₁-C₆ alkyl-NR¹—C₁-C₆ alkyl, NR³R⁴, —C(O)NR³R⁴, —S(O)C₁-C₆ alkyl, —S(O)₂NR³R⁴, and —S(O)₂C₁-C₆ alkyl; and all other variables are as defined herein.

In some embodiments, the compound is of Formula (A-1-a-I-C-1-c-III):

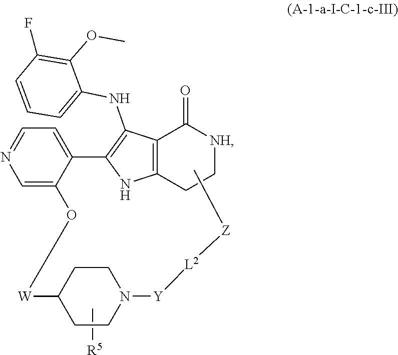

or a pharmaceutically acceptable salt, prodrug, stereoisomer, solvate, or tautomer thereof, wherein W is (CH₂)_{w}; w is an integer selected from 0, 1, 2, 3, 4, 5, 6, 7, 8, 9, and 10; R⁵ is selected from H, halogen, OH, oxo, CN, C₁-C₆ alkyl, C₁-C₆ alkoxy, C₁-C₆ alkyl-C₁-C₆ alkoxy, C₁-C₆ alkyl-NR¹—C₁-C₆ alkyl, NR³R⁴, —C(O)NR³R⁴, —S(O)C₁-C₆ alkyl, —S(O)₂NR³R⁴, and —S(O)₂C₁-C₆ alkyl; and all other variables are as defined herein.

In some embodiments, the compound is of Formula (A-1-a-I-C-2):

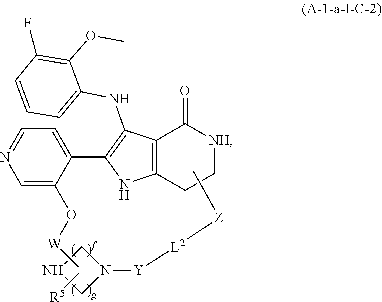

or a pharmaceutically acceptable salt, prodrug, stereoisomer, solvate, or tautomer thereof, wherein W is (CH₂)_{w}; w is an integer selected from 0, 1, 2, 3, 4, 5, 6, 7, 8, 9, and 10; f is an integer selected from 1, 2, and 3; g is an integer selected from 1, 2, and 3; R⁵ is selected from H, halogen, OH, oxo, CN, C₁-C₆ alkyl, C₁-C₆ alkoxy, C₁-C₆ alkyl-C₁-C₆ alkoxy, C₁-C₆ alkyl-NR¹—C₁-C₆ alkyl, NR³R⁴, —C(O)NR³R⁴, —S(O)C₁-C₆ alkyl, —S(O)₂NR³R⁴, and —S(O)₂C₁-C₆ alkyl; and all other variables are as defined herein.

In some embodiments, the compound is of Formula (A-1-a-I-C-2-a):

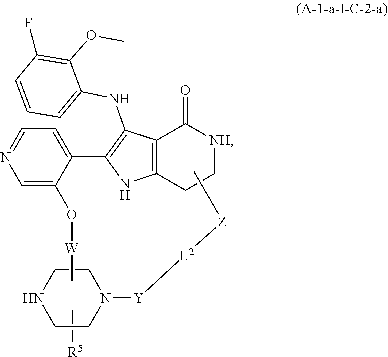

or a pharmaceutically acceptable salt, prodrug, stereoisomer, solvate, or tautomer thereof, wherein W is (CH₂)_{w}; w is an integer selected from 0, 1, 2, 3, 4, 5, 6, 7, 8, 9, and 10; R⁵ is selected from H, halogen, OH, oxo, CN, C₁-C₆ alkyl, C₁-C₆ alkoxy, C₁-C₆ alkyl-C₁-C₆ alkoxy, C₁-C₆ alkyl-NR¹—C₁-C₆ alkyl, NR³R⁴, —C(O)NR³R⁴, —S(O)C₁-C₆ alkyl, —S(O)₂NR³R⁴, and —S(O)₂C₁-C₆ alkyl; and all other variables are as defined herein.

In some embodiments, the compound is of Formula (A-1-a-I-C-2-a-I):

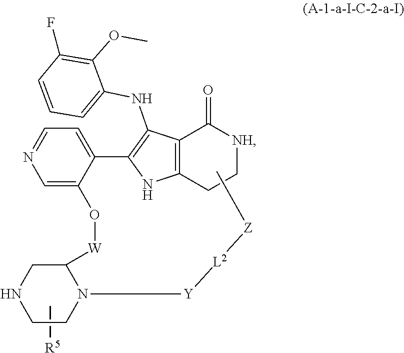

or a pharmaceutically acceptable salt, prodrug, stereoisomer, solvate, or tautomer thereof, wherein W is (CH₂)_{w}; w is an integer selected from 0, 1, 2, 3, 4, 5, 6, 7, 8, 9, and 10; R⁵ is selected from H, halogen, OH, oxo, CN, C₁-C₆ alkyl, C₁-C₆ alkoxy, C₁-C₆ alkyl-C₁-C₆ alkoxy, C₁-C₆ alkyl-NR¹—C₁-C₆ alkyl, NR³R⁴, —C(O)NR³R⁴, —S(O)C₁-C₆ alkyl, —S(O)₂NR³R⁴, and —S(O)₂C₁-C₆ alkyl; and all other variables are as defined herein.

In some embodiments, the compound is of Formula (A-1-a-I-C-2-a-II):

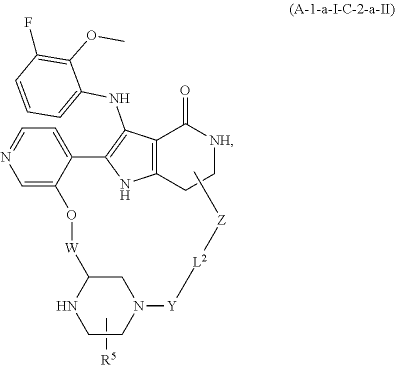

or a pharmaceutically acceptable salt, prodrug, stereoisomer, solvate, or tautomer thereof, wherein W is (CH₂)_{w}; w is an integer selected from 0, 1, 2, 3, 4, 5, 6, 7, 8, 9, and 10; R⁵ is selected from H, halogen, OH, oxo, CN, C₁-C₆ alkyl, C₁-C₆ alkoxy, C₁-C₆ alkyl-C₁-C₆ alkoxy, C₁-C₆ alkyl-NR¹—C₁-C₆ alkyl, NR³R⁴, —C(O)NR³R⁴, —S(O)C₁-C₆ alkyl, —S(O)₂NR³R⁴, and —S(O)₂C₁-C₆ alkyl; and all other variables are as defined herein.

In some embodiments, the compound is of Formula (A-1-a-I-C-2-a-III):

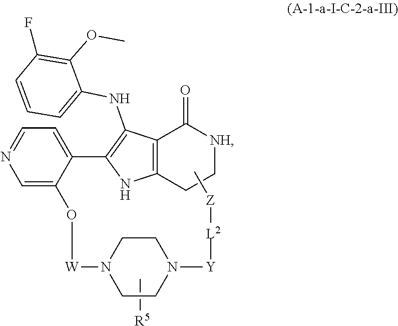

or a pharmaceutically acceptable salt, prodrug, stereoisomer, solvate, or tautomer thereof, wherein W is (CH₂)_{w}; w is an integer selected from 0, 1, 2, 3, 4, 5, 6, 7, 8, 9, and 10; R⁵ is selected from H, halogen, OH, oxo, CN, C₁-C₆ alkyl, C₁-C₆ alkoxy, C₁-C₆ alkyl-C₁-C₆ alkoxy, C₁-C₆ alkyl-NR¹—C₁-C₆ alkyl, NR³R⁴, —C(O)NR³R⁴, —S(O)C₁-C₆ alkyl, —S(O)₂NR³R⁴, and —S(O)₂C₁-C₆ alkyl; and all other variables are as defined herein.

In some embodiments, the compound is of Formula (A-1-a-I-C-3):

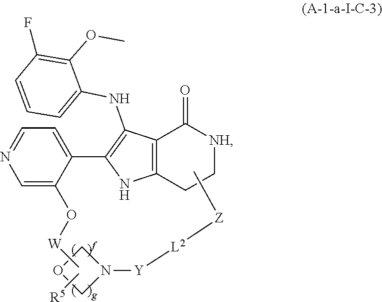

or a pharmaceutically acceptable salt, prodrug, stereoisomer, solvate, or tautomer thereof, wherein W is (CH₂)_{w}; w is an integer selected from 0, 1, 2, 3, 4, 5, 6, 7, 8, 9, and 10; f is an integer selected from 1, 2, and 3; g is an integer selected from 1, 2, and 3; R⁵ is selected from H, halogen, OH, oxo, CN, C₁-C₆ alkyl, C₁-C₆ alkoxy, C₁-C₆ alkyl-C₁-C₆ alkoxy, C₁-C₆ alkyl-NR¹—C₁-C₆ alkyl, NR³R⁴, —C(O)NR³R⁴, —S(O)C₁-C₆ alkyl, —S(O)₂NR³R⁴, and —S(O)₂C₁-C₆ alkyl; and all other variables are as defined herein.

In some embodiments, the compound is of Formula (A-1-a-I-C-3-a):

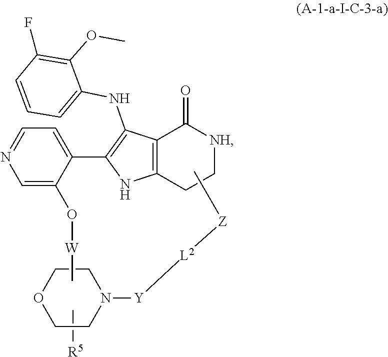

or a pharmaceutically acceptable salt, prodrug, stereoisomer, solvate, or tautomer thereof, wherein W is (CH₂)_{w}; w is an integer selected from 0, 1, 2, 3, 4, 5, 6, 7, 8, 9, and 10; R⁵ is selected from H, halogen, OH, oxo, CN, C₁-C₆ alkyl, C₁-C₆ alkoxy, C₁-C₆ alkyl-C₁-C₆ alkoxy, C₁-C₆ alkyl-NR¹—C₁-C₆ alkyl, NR³R⁴, —C(O)NR³R⁴, —S(O)C₁-C₆ alkyl, —S(O)₂NR³R⁴, and —S(O)₂C₁-C₆ alkyl; and all other variables are as defined herein.

In some embodiments, the compound is of Formula (A-1-a-I-C-3-a-I):

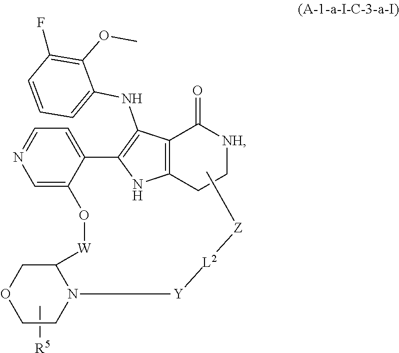

or a pharmaceutically acceptable salt, prodrug, stereoisomer, solvate, or tautomer thereof, wherein W is (CH₂)_{w}; w is an integer selected from 0, 1, 2, 3, 4, 5, 6, 7, 8, 9, and 10; R⁵ is selected from H, halogen, OH, oxo, CN, C₁-C₆ alkyl, C₁-C₆ alkoxy, C₁-C₆ alkyl-C₁-C₆ alkoxy, C₁-C₆ alkyl-NR¹—C₁-C₆ alkyl, NR³R⁴, —C(O)NR³R⁴, —S(O)C₁-C₆ alkyl, —S(O)₂NR³R⁴, and —S(O)₂C₁-C₆ alkyl; and all other variables are as defined herein.

In some embodiments, the compound is of Formula (A-1-a-I-C-3-a-II):

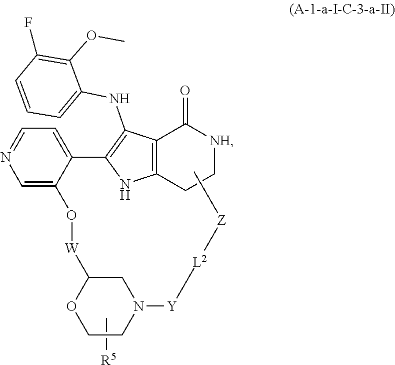

or a pharmaceutically acceptable salt, prodrug, stereoisomer, solvate, or tautomer thereof, wherein W is (CH₂)_{w}; w is an integer selected from 0, 1, 2, 3, 4, 5, 6, 7, 8, 9, and 10; R⁵ is selected from H, halogen, OH, oxo, CN, C₁-C₆ alkyl, C₁-C₆ alkoxy, C₁-C₆ alkyl-C₁-C₆ alkoxy, C₁-C₆ alkyl-NR¹—C₁-C₆ alkyl, NR³R⁴, —C(O)NR³R⁴, —S(O)C₁-C₆ alkyl, —S(O)₂NR³R⁴, and —S(O)₂C₁-C₆ alkyl; and all other variables are as defined herein.

In some embodiments, the compound is of Formula (A-1-a-I-D):

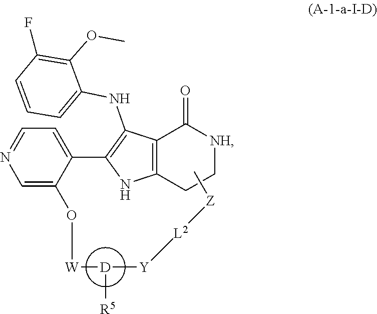

or a pharmaceutically acceptable salt, prodrug, stereoisomer, solvate, or tautomer thereof, wherein Ring D is 5-10 membered monocyclic or bicyclic heteroaryl comprising 1-3 heteroatoms selected from N, O, S; W is (CH₂)_{w}; w is an integer selected from 0, 1, 2, 3, 4, 5, 6, 7, 8, 9, and 10; R⁵ is selected from H, halogen, OH, oxo, CN, C₁-C₆ alkyl, C₁-C₆ alkoxy, C₁-C₆ alkyl-C₁-C₆ alkoxy, C₁-C₆ alkyl-NR¹—C₁-C₆ alkyl, NR³R⁴, —C(O)NR³R⁴, —S(O)C₁-C₆ alkyl, —S(O)₂NR³R⁴, and —S(O)₂C₁-C₆ alkyl and all other variables are as defined herein.

In some embodiments, the compound is of Formula (A-1-a-I-D-1):

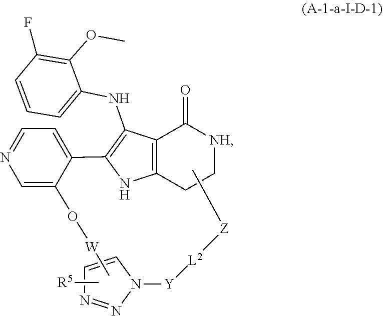

or a pharmaceutically acceptable salt, prodrug, stereoisomer, solvate, or tautomer thereof, wherein W is (CH₂)_{w}; w is an integer selected from 0, 1, 2, 3, 4, 5, 6, 7, 8, 9, and 10; R⁵ is selected from H, halogen, OH, oxo, CN, C₁-C₆ alkyl, C₁-C₆ alkoxy, C₁-C₆ alkyl-C₁-C₆ alkoxy, C₁-C₆ alkyl-NR¹—C₁-C₆ alkyl, NR³R⁴, —C(O)NR³R⁴, —S(O)C₁-C₆ alkyl, —S(O)₂NR³R⁴, and —S(O)₂C₁-C₆ alkyl and all other variables are as defined herein.

In some embodiments, the compound is of Formula (A-1-a-I-D-1-a):

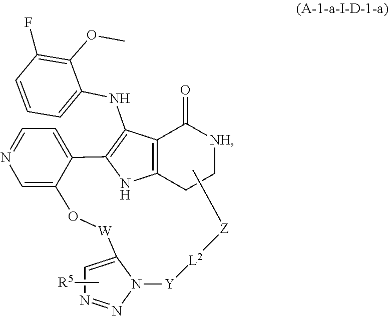

or a pharmaceutically acceptable salt, prodrug, stereoisomer, solvate, or tautomer thereof, wherein W is (CH₂)_{w}; w is an integer selected from 0, 1, 2, 3, 4, 5, 6, 7, 8, 9, and 10; R⁵ is selected from H, halogen, OH, oxo, CN, C₁-C₆ alkyl, C₁-C₆ alkoxy, C₁-C₆ alkyl-C₁-C₆ alkoxy, C₁-C₆ alkyl-NR¹—C₁-C₆ alkyl, NR³R⁴, —C(O)NR³R⁴, —S(O)C₁-C₆ alkyl, —S(O)₂NR³R⁴, and —S(O)₂C₁-C₆ alkyl and all other variables are as defined herein.

In some embodiments, the compound is of Formula (A-1-a-I-D-1-b):

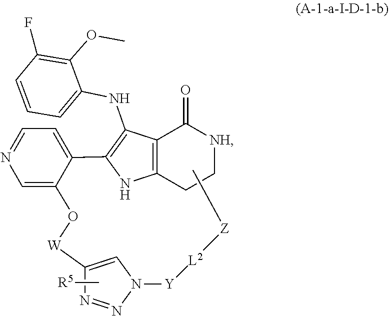

or a pharmaceutically acceptable salt, prodrug, stereoisomer, solvate, or tautomer thereof, wherein W is (CH₂)_{w}; w is an integer selected from 0, 1, 2, 3, 4, 5, 6, 7, 8, 9, and 10; R⁵ is selected from H, halogen, OH, oxo, CN, C₁-C₆ alkyl, C₁-C₆ alkoxy, C₁-C₆ alkyl-C₁-C₆ alkoxy, C₁-C₆ alkyl-NR¹—C₁-C₆ alkyl, NR³R⁴, —C(O)NR³R⁴, —S(O)C₁-C₆ alkyl, —S(O)₂NR³R⁴, and —S(O)₂C₁-C₆ alkyl and all other variables are as defined herein.

In some embodiments, the compound is of Formula (A-1-b-I):

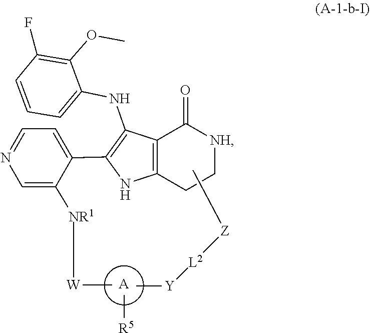

or a pharmaceutically acceptable salt, prodrug, stereoisomer, solvate, or tautomer thereof, wherein Ring A is selected from C₃-C₁₀-cycloalkane, arene, heterocycle, heteroarene; W is (CH₂)_{w}; w is an integer selected from 0, 1, 2, 3, 4, 5, 6, 7, 8, 9, and 10; R⁵ is selected from H, halogen, OH, oxo, CN, C₁-C₆ alkyl, C₁-C₆ alkoxy, C₁-C₆ alkyl-C₁-C₆ alkoxy, C₁-C₆ alkyl-NR¹—C₁-C₆ alkyl, NR³R⁴, —C(O)NR³R⁴, —S(O)C₁-C₆ alkyl, —S(O)₂NR³R⁴, and —S(O)₂C₁-C₆ alkyl and all other variables are as defined herein.

In some embodiments, the compound is of Formula (A-1-b-I-A):

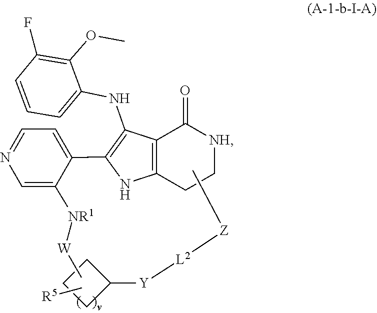

or a pharmaceutically acceptable salt, prodrug, stereoisomer, solvate, or tautomer thereof, wherein W is (CH₂)_{w}; w is an integer selected from 0, 1, 2, 3, 4, 5, 6, 7, 8, 9, and 10; v is an integer selected from 0, 1, 2, 3, 4 and 5; R⁵ is selected from H, halogen, OH, oxo, CN, C₁-C₆ alkyl, C₁-C₆ alkoxy, C₁-C₆ alkyl-C₁-C₆ alkoxy, C₁-C₆ alkyl-NR¹—C₁-C₆ alkyl, NR³R⁴, —C(O)NR³R⁴, —S(O)C₁-C₆ alkyl, —S(O)₂NR³R⁴, and —S(O)₂C₁-C₆ alkyl; and all other variables are as defined herein.

In some embodiments, the compound is of Formula (A-1-b-I-A-1):

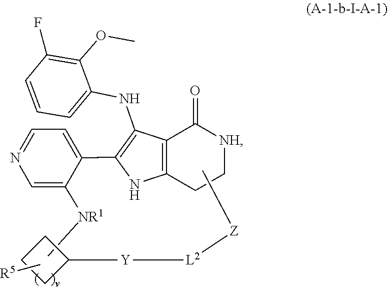

or a pharmaceutically acceptable salt, prodrug, stereoisomer, solvate, or tautomer thereof, wherein v is an integer selected from 0, 1, 2, 3, 4 and 5; R⁵ is selected from H, halogen, OH, oxo, CN, C₁-C₆ alkyl, C₁-C₆ alkoxy, C₁-C₆ alkyl-C₁-C₆ alkoxy, C₁-C₆ alkyl-NR¹—C₁-C₆ alkyl, NR³R⁴, —C(O)NR³R⁴, —S(O)C₁-C₆ alkyl, —S(O)₂NR³R⁴, and —S(O)₂C₁-C₆ alkyl; and all other variables are as defined herein.

In some embodiments, the compound is of Formula (A-1-b-I-B):

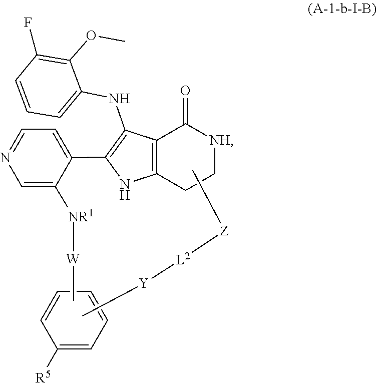

or a pharmaceutically acceptable salt, prodrug, stereoisomer, solvate, or tautomer thereof, wherein W is (CH₂)_{w}; w is an integer selected from 0, 1, 2, 3, 4, 5, 6, 7, 8, 9, and 10; R⁵ is selected from H, halogen, OH, oxo, CN, C₁-C₆ alkyl, C₁-C₆ alkoxy, C₁-C₆ alkyl-C₁-C₆ alkoxy, C₁-C₆ alkyl-NR¹—C₁-C₆ alkyl, NR³R⁴, —C(O)NR³R⁴, —S(O)C₁-C₆ alkyl, —S(O)₂NR³R⁴, and —S(O)₂C₁-C₆ alkyl and all other variables are as defined herein.

In some embodiments, the compound is of Formula (A-1-b-I-B-0):

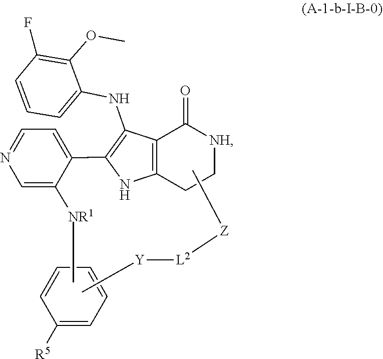

or a pharmaceutically acceptable salt, prodrug, stereoisomer, solvate, or tautomer thereof, wherein R⁵ is selected from H, halogen, OH, oxo, CN, C₁-C₆ alkyl, C₁-C₆ alkoxy, C₁-C₆ alkyl-C₁-C₆ alkoxy, C₁-C₆ alkyl-NR¹—C₁-C₆ alkyl, NR³R⁴, —C(O)NR³R⁴, —S(O)C₁-C₆ alkyl, —S(O)₂NR³R⁴, and —S(O)₂C₁-C₆ alkyl and all other variables are as defined herein.

In some embodiments, the compound is of Formula (A-1-b-I-B-1):

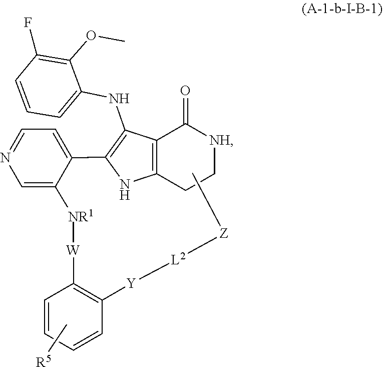

or a pharmaceutically acceptable salt, prodrug, stereoisomer, solvate, or tautomer thereof, wherein W is (CH₂)_{w}; w is an integer selected from 0, 1, 2, 3, 4, 5, 6, 7, 8, 9, and 10; R⁵ is selected from H, halogen, OH, oxo, CN, C₁-C₆ alkyl, C₁-C₆ alkoxy, C₁-C₆ alkyl-C₁-C₆ alkoxy, C₁-C₆ alkyl-NR¹—C₁-C₆ alkyl, NR³R⁴, —C(O)NR³R⁴, —S(O)C₁-C₆ alkyl, —S(O)₂NR³R⁴, and —S(O)₂C₁-C₆ alkyl and all other variables are as defined herein.

In some embodiments, the compound is of Formula (A-1-b-I-B-2):

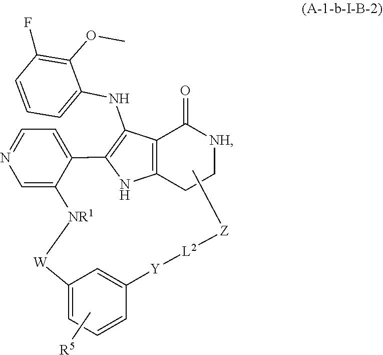

or a pharmaceutically acceptable salt, prodrug, stereoisomer, solvate, or tautomer thereof, wherein W is (CH₂)_{w}; w is an integer selected from 0, 1, 2, 3, 4, 5, 6, 7, 8, 9, and 10; R⁵ is selected from H, halogen, OH, oxo, CN, C₁-C₆ alkyl, C₁-C₆ alkoxy, C₁-C₆ alkyl-C₁-C₆ alkoxy, C₁-C₆ alkyl-NR¹—C₁-C₆ alkyl, NR³R⁴, —C(O)NR³R⁴, —S(O)C₁-C₆ alkyl, —S(O)₂NR³R⁴, and —S(O)₂C₁-C₆ alkyl and all other variables are as defined herein.

In some embodiments, the compound is of Formula (A-1-b-I-B-3):

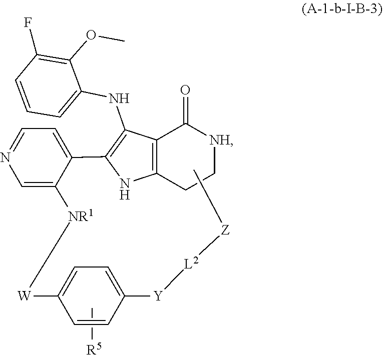

or a pharmaceutically acceptable salt, pro rug, s ereoisomer, solvate, or automer thereof, wherein W is (CH₂)_{w}; w is an integer selected from 0, 1, 2, 3, 4, 5, 6, 7, 8, 9, and 10; R⁵ is selected from H, halogen, OH, oxo, CN, C₁-C₆ alkyl, C₁-C₆ alkoxy, C₁-C₆ alkyl-C₁-C₆ alkoxy, C₁-C₆ alkyl-NR¹—C₁-C₆ alkyl, NR³R⁴, —C(O)NR³R⁴, —S(O)C₁-C₆ alkyl, —S(O)₂NR³R⁴, and —S(O)₂C₁-C₆ alkyl and all other variables are as defined herein.

In some embodiments, the compound is of Formula (A-1-b-I-C):

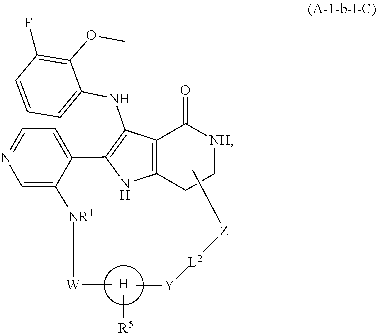

or a pharmaceutically acceptable salt, prodrug, stereoisomer, solvate, or tautomer thereof, wherein Ring H is 3-10 membered monocyclic or bicyclic heterocycle comprising 1-3 heteroatoms selected from N, O, S; W is (CH₂)_{w}; w is an integer selected from 0, 1, 2, 3, 4, 5, 6, 7, 8, 9, and 10; R⁵ is selected from H, halogen, OH, oxo, CN, C₁-C₆ alkyl, C₁-C₆ alkoxy, C₁-C₆ alkyl-C₁-C₆ alkoxy, C₁-C₆ alkyl-NR¹—C₁-C₆ alkyl, NR³R⁴, —C(O)NR³R⁴, —S(O)C₁-C₆ alkyl, —S(O)₂NR³R⁴, and —S(O)₂C₁-C₆ alkyl and all other variables are as defined herein.

In some embodiments, the compound is of Formula (A-1-b-I-C-1):

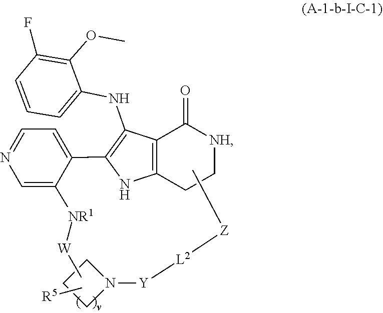

or a pharmaceutically acceptable salt, prodrug, stereoisomer, solvate, or tautomer thereof, wherein W is (CH₂)_{w}; w is an integer selected from 0, 1, 2, 3, 4, 5, 6, 7, 8, 9, and 10; v is an integer selected from 0, 1, 2, 3, 4 and 5; R⁵ is selected from H, halogen, OH, oxo, CN, C₁-C₆ alkyl, C₁-C₆ alkoxy, C₁-C₆ alkyl-C₁-C₆ alkoxy, C₁-C₆ alkyl-NR¹—C₁-C₆ alkyl, NR³R⁴, —C(O)NR³R⁴, —S(O)C₁-C₆ alkyl, —S(O)₂NR³R⁴, and —S(O)₂C₁-C₆ alkyl; and all other variables are as defined herein.

In some embodiments, the compound is of Formula (A-1-b-I-C-1-a):

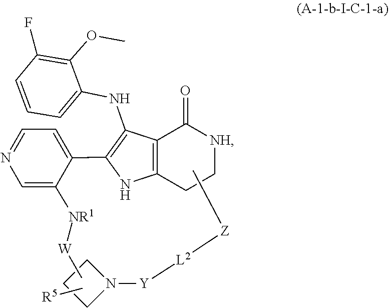

or a pharmaceutically acceptable salt, prodrug, stereoisomer, solvate, or tautomer thereof, wherein W is (CH₂)_{w}; w is an integer selected from 0, 1, 2, 3, 4, 5, 6, 7, 8, 9, and 10; R⁵ is selected from H, halogen, OH, oxo, CN, C₁-C₆ alkyl, C₁-C₆ alkoxy, C₁-C₆ alkyl-C₁-C₆ alkoxy, C₁-C₆ alkyl-NR¹—C₁-C₆ alkyl, NR³R⁴, —C(O)NR³R⁴, —S(O)C₁-C₆ alkyl, —S(O)₂NR³R⁴, and —S(O)₂C₁-C₆ alkyl; and all other variables are as defined herein.

In some embodiments, the compound is of Formula (A-1-b-I-C-1-a-I):

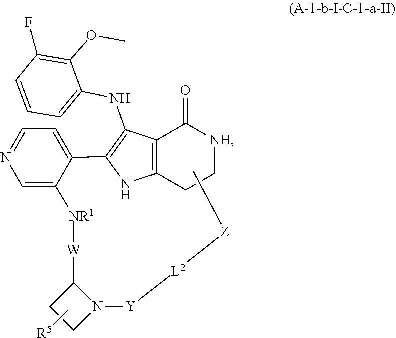

or a pharmaceutically acceptable salt, prodrug, stereoisomer, solvate, or tautomer thereof, wherein W is (CH₂)_{w}; w is an integer selected from 0, 1,2, 3,4, 5, 6, 7,8,9, and 10; Ri is selected from H, halogen, OH, oxo, CN, C₁-C₆ alkyl, C₁-C₆ alkoxy, C₁-C₆ alkyl-C₁-C₆ alkoxy, C₁-C₆ alkyl-NR¹—C₁-C₆ alkyl, NR³R⁴, —C(O)NR³R⁴, —S(O)C₁-C₆ alkyl, —S(O)₂NR³R⁴, and —S(O)₂C₁-C₆ alkyl; and all other variables are as defined herein.

In some embodiments, the compound is of Formula (A-1-b-I-C-1-a-II):

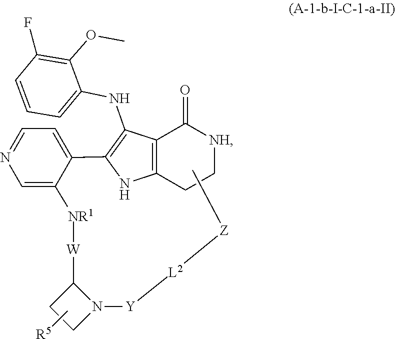

or a pharmaceutically acceptable salt, prodrug, stereoisomer, solvate, or tautomer thereof, wherein W is (CH₂)_{w}; w is an integer selected from 0, i, 2, 3,4, 5,6, 7, 8,9, and 10; R¹ is selected from H, halogen, OH, oxo, CN, C₁-C₆ alkyl, C₁-C₆ alkoxy, C₁-C₆ alkyl-C₁-C₆ alkoxy, C₁-C₆ alkyl-NR¹—C₁-C₆ alkyl, NR³R⁴, —C(O)NR³R⁴, —S(O)C₁-C₆ alkyl, —S(O)₂NR³R⁴, and —S(O)₂C₁-C₆ alkyl; and all other variables are as defined herein.

In some embodiments, the compound is of Formula (A-i-b-I-C-i-b):

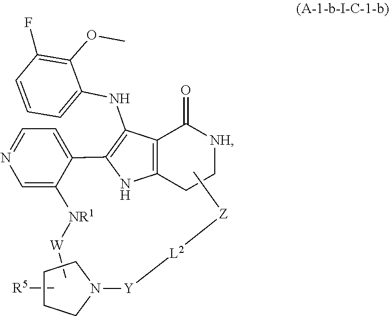

or a pharmaceutically acceptable salt, prodrug, stereoisomer, solvate, or tautomer thereof, wherein W is (CH₂)_{w}; w is an integer selected from 0, 1, 2, 3, 4, 5, 6, 7, 8, 9, and 10; R⁵ is selected from H, halogen, OH, oxo, CN, C₁-C₆ alkyl, C₁-C₆ alkoxy, C₁-C₆ alkyl-C₁-C₆ alkoxy, C₁-C₆ alkyl-NR¹—C₁-C₆ alkyl, NR³R⁴, —C(O)NR³R⁴, —S(O)C₁-C₆ alkyl, —S(O)₂NR³R⁴, and —S(O)₂C₁-C₆ alkyl; and all other variables are as defined herein.

In some embodiments, the compound is of Formula (A-1-b-I-C-1-b-I):

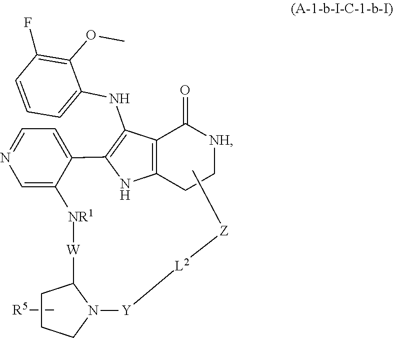

or a pharmaceutically acceptable salt, prodrug, stereoisomer, solvate, or tautomer thereof, wherein W is (CH₂)_{w}; w is an integer selected from 0, 1, 2, 3, 4, 5, 6, 7, 8, 9, and 10; R⁵ is selected from H, halogen, OH, oxo, CN, C₁-C₆ alkyl, C₁-C₆ alkoxy, C₁-C₆ alkyl-C₁-C₆ alkoxy, C₁-C₆ alkyl-NR¹—C₁-C₆ alkyl, NR³R⁴, —C(O)NR³R⁴, —S(O)C₁-C₆ alkyl, —S(O)₂NR³R⁴, and —S(O)₂C₁-C₆ alkyl; and all other variables are as defined herein.

In some embodiments, the compound is of Formula (A-1-b-I-C-1-b-II):

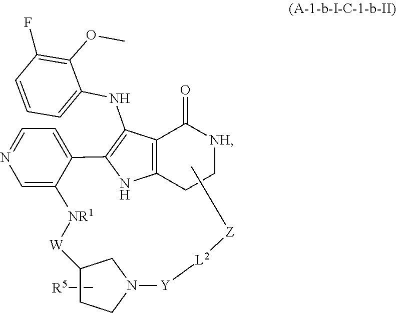

or a pharmaceutically acceptable salt, prodrug, stereoisomer, solvate, or tautomer thereof, wherein W is (CH₂)_{w}; w is an integer selected from 0, 1, 2, 3, 4, 5, 6, 7, 8, 9, and 10; R⁵ is selected from H, halogen, OH, oxo, CN, C₁-C₆ alkyl, C₁-C₆ alkoxy, C₁-C₆ alkyl-C₁-C₆ alkoxy, C₁-C₆ alkyl-NR¹—C₁-C₆ alkyl, NR³R⁴, —C(O)NR³R⁴, —S(O)C₁-C₆ alkyl, —S(O)₂NR³R⁴, and —S(O)₂C₁-C₆ alkyl; and all other variables are as defined herein.

In some embodiments, the compound is of Formula (A-1-b-I-C-1-c):

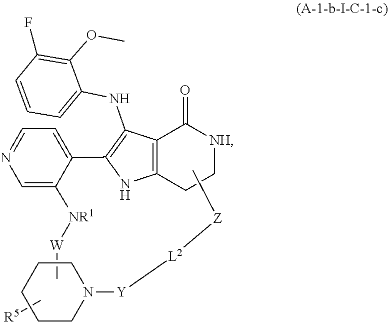

or a pharmaceutically acceptable salt, prodrug, stereoisomer, solvate, or tautomer thereof, wherein W is (CH₂)_{w}; w is an integer selected from 0, 1, 2, 3, 4, 5, 6, 7, 8, 9, and 10; R⁵ is selected from H, halogen, OH, oxo, CN, C₁-C₆ alkyl, C₁-C₆ alkoxy, C₁-C₆ alkyl-C₁-C₆ alkoxy, C₁-C₆ alkyl-NR¹—C₁-C₆ alkyl, NR³R⁴, —C(O)NR³R⁴, —S(O)C₁-C₆ alkyl, —S(O)₂NR³R⁴, and —S(O)₂C₁-C₆ alkyl; and all other variables are as defined herein.

In some embodiments, the compound is of Formula (A-1-b-I-C-1-c-I):

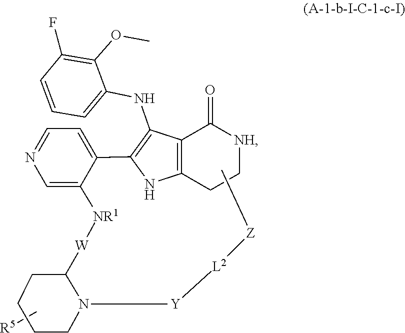

or a pharmaceutically acceptable salt, prodrug, stereoisomer, solvate, or tautomer thereof, wherein W is (CH₂)_{w}; w is an integer selected from 0, 1, 2, 3, 4, 5, 6, 7, 8, 9, and 10; R⁵ is selected from H, halogen, OH, oxo, CN, C₁-C₆ alkyl, C₁-C₆ alkoxy, C₁-C₆ alkyl-C₁-C₆ alkoxy, C₁-C₆ alkyl-NR¹—C₁-C₆ alkyl, NR³R⁴, —C(O)NR³R⁴, —S(O)C₁-C₆ alkyl, —S(O)₂NR³R⁴, and —S(O)₂C₁-C₆ alkyl; and all other variables are as defined herein.

In some embodiments, the compound is of Formula (A-1-b-I-C-1-c-II):

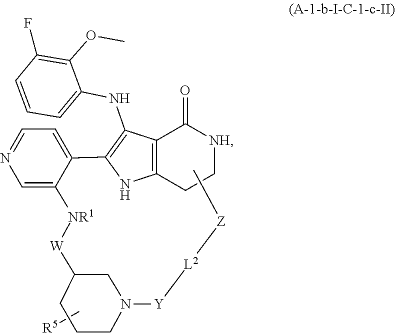

or a pharmaceutically acceptable salt, prodrug, stereoisomer, solvate, or tautomer thereof, wherein W is (CH₂)_{w}; w is an integer selected from 0, 1, 2, 3, 4, 5, 6, 7, 8, 9, and 10; R⁵ is selected from H, halogen, OH, oxo, CN, C₁-C₆ alkyl, C₁-C₆ alkoxy, C₁-C₆ alkyl-C₁-C₆ alkoxy, C₁-C₆ alkyl-NR¹—C₁-C₆ alkyl, NR³R⁴, —C(O)NR³R⁴, —S(O)C₁-C₆ alkyl, —S(O)₂NR³R⁴, and —S(O)₂C₁-C₆ alkyl; and all other variables are as defined herein.

In some embodiments, the compound is of Formula (A-1-b-I-C-1-c-III):

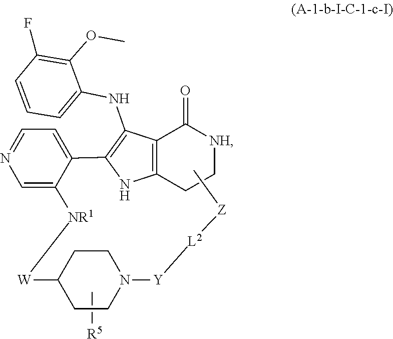

or a pharmaceutically acceptable salt, prodrug, stereoisomer, solvate, or tautomer thereof, wherein W is (CH₂)_{w}; w is an integer selected from 0, 1, 2, 3, 4, 5, 6, 7, 8, 9, and 10; R⁵ is selected from H, halogen, OH, oxo, CN, C₁-C₆ alkyl, C₁-C₆ alkoxy, C₁-C₆ alkyl-C₁-C₆ alkoxy, C₁-C₆ alkyl-NR¹—C₁-C₆ alkyl, NR³R⁴, —C(O)NR³R⁴, —S(O)C₁-C₆ alkyl, —S(O)₂NR³R⁴, and —S(O)₂C₁-C₆ alkyl; and all other variables are as defined herein.

In some embodiments, the compound is of Formula (A-1-b-I-C-2):

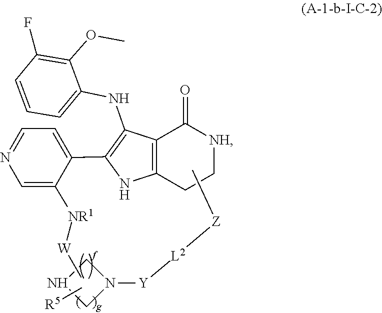

or a pharmaceutically acceptable salt, prodrug, stereoisomer, solvate, or tautomer thereof, wherein W is (CH₂)_{w}; w is an integer selected from 0, 1, 2, 3, 4, 5, 6, 7, 8, 9, and 10; f is an integer selected from 1, 2, and 3; g is an integer selected from 1, 2, and 3; R⁵ is selected from H, halogen, OH, oxo, CN, C₁-C₆ alkyl, C₁-C₆ alkoxy, C₁-C₆ alkyl-C₁-C₆ alkoxy, C₁-C₆ alkyl-NR¹—C₁-C₆ alkyl, NR³R⁴, —C(O)NR³R⁴, —S(O)C₁-C₆ alkyl, —S(O)₂NR³R⁴, and —S(O)₂C₁-C₆ alkyl; and all other variables are as defined herein.

In some embodiments, the compound is of Formula (A-1-b-I-C-2-a):

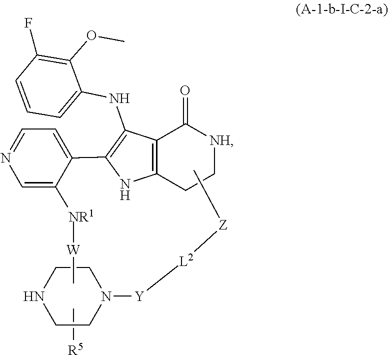

or a pharmaceutically acceptable salt, prodrug, stereoisomer, solvate, or tautomer thereof, wherein W is (CH₂)_{w}; w is an integer selected from 0, 1, 2, 3, 4, 5, 6, 7, 8, 9, and 10; R⁵ is selected from H, halogen, OH, oxo, CN, C₁-C₆ alkyl, C₁-C₆ alkoxy, C₁-C₆ alkyl-C₁-C₆ alkoxy, C₁-C₆ alkyl-NR¹—C₁-C₆ alkyl, NR³R⁴, —C(O)NR³R⁴, —S(O)C₁-C₆ alkyl, —S(O)₂NR³R⁴, and —S(O)₂C₁-C₆ alkyl; and all other variables are as defined herein.

In some embodiments, the compound is of Formula (A-1-b-I-C-2-a-I):

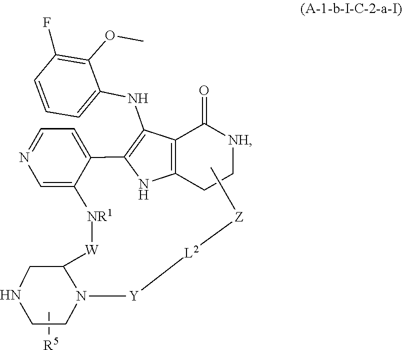

or a pharmaceutically acceptable salt, prodrug, stereoisomer, solvate, or tautomer thereof, wherein W is (CH₂)_{w}; w is an integer selected from 0, 1, 2, 3, 4, 5, 6, 7, 8, 9, and 10; R⁵ is selected from H, halogen, OH, oxo, CN, C₁-C₆ alkyl, C₁-C₆ alkoxy, C₁-C₆ alkyl-C₁-C₆ alkoxy, C₁-C₆ alkyl-NR¹—C₁-C₆ alkyl, NR³R⁴, —C(O)NR³R⁴, —S(O)C₁-C₆ alkyl, —S(O)₂NR³R⁴, and —S(O)₂C₁-C₆ alkyl; and all other variables are as defined herein.

In some embodiments, the compound is of Formula (A-1-b-I-C-2-a-II):

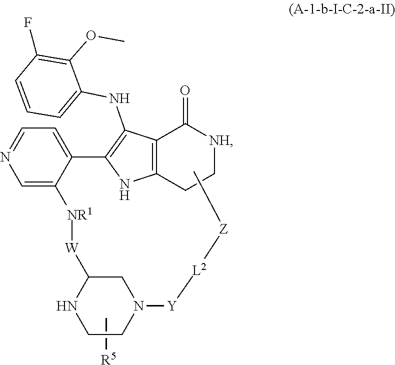

or a pharmaceutically acceptable salt, pro rug, stereoisomer, solvate, or tautomer thereof, wherein W is (CH₂)_{w}; w is an integer selected from 0, 1, 2, 3, 4, 5, 6, 7, 8, 9, and 10; R⁵ is selected from H, halogen, OH, oxo, CN, C₁-C₆ alkyl, C₁-C₆ alkoxy, C₁-C₆ alkyl-C₁-C₆ alkoxy, C₁-C₆ alkyl-NR¹—C₁-C₆ alkyl, NR³R⁴, —C(O)NR³R⁴, —S(O)C₁-C₆ alkyl, —S(O)₂NR³R⁴, and —S(O)₂C₁-C₆ alkyl; and all other variables are as defined herein.

In some embodiments, the compound is of Formula (A-1-b-I-C-2-a-III):

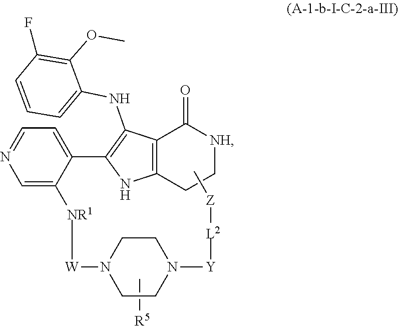

or a pharmaceutically acceptable salt, prodrug, stereoisomer, solvate, or tautomer thereof, wherein W is (CH₂)_{w}; w is an integer selected from 0, 1, 2, 3, 4, 5, 6, 7, 8, 9, and 10; R⁵ is selected from H, halogen, OH, oxo, CN, C₁-C₆ alkyl, C₁-C₆ alkoxy, C₁-C₆ alkyl-C₁-C₆ alkoxy, C₁-C₆ alkyl-NR¹—C₁-C₆ alkyl, NR³R⁴, —C(O)NR³R⁴, —S(O)C₁-C₆ alkyl, —S(O)₂NR³R⁴, and —S(O)₂C₁-C₆ alkyl; and all other variables are as defined herein.

In some embodiments, the compound is of Formula (A-1-b-I-C-3):

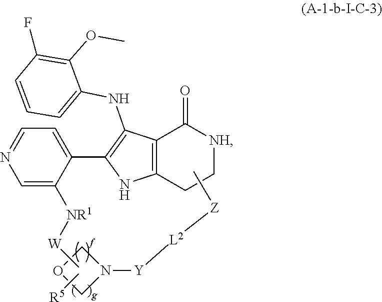

or a pharmaceutically acceptable salt, prodrug, stereoisomer, solvate, or tautomer thereof, wherein W is (CH₂)_{w}; w is an integer selected from 0, 1, 2, 3, 4, 5, 6, 7, 8, 9, and 10; f is an integer selected from 1, 2, and 3; g is an integer selected from 1, 2, and 3; R⁵ is selected from H, halogen, OH, oxo, CN, C₁-C₆ alkyl, C₁-C₆ alkoxy, C₁-C₆ alkyl-C₁-C₆ alkoxy, C₁-C₆ alkyl-NR¹—C₁-C₆ alkyl, NR³R⁴, —C(O)NR³R⁴, —S(O)C₁-C₆ alkyl, —S(O)₂NR³R⁴, and —S(O)₂C₁-C₆ alkyl; and all other variables are as defined herein.

In some embodiments, the compound is of Formula (A-1-b-I-C-3-a):

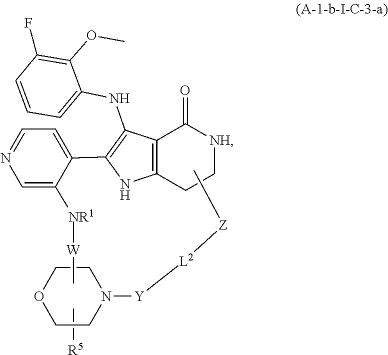

or a pharmaceutically acceptable salt, prodrug, stereoisomer, solvate, or tautomer thereof, wherein W is (CH₂)_{w}; w is an integer selected from 0, 1, 2, 3, 4, 5, 6, 7, 8, 9, and 10; R⁵ is selected from H, halogen, OH, oxo, CN, C₁-C₆ alkyl, C₁-C₆ alkoxy, C₁-C₆ alkyl-C₁-C₆ alkoxy, C₁-C₆ alkyl-NR¹—C₁-C₆ alkyl, NR³R⁴, —C(O)NR³R⁴, —S(O)C₁-C₆ alkyl, —S(O)₂NR³R⁴, and —S(O)₂C₁-C₆ alkyl; and all other variables are as defined herein.

In some embodiments, the compound is of Formula (A-1-b-I-C-3-a-I):

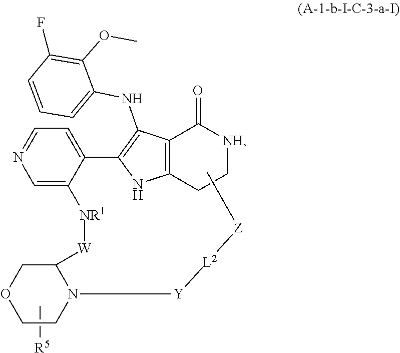

or a pharmaceutically acceptable salt, prodrug, stereoisomer, solvate, or tautomer thereof, wherein W is (CH₂)_{w}; w is an integer selected from 0, 1, 2, 3, 4, 5, 6, 7, 8, 9, and 10; R⁵ is selected from H, halogen, OH, oxo, CN, C₁-C₆ alkyl, C₁-C₆ alkoxy, C₁-C₆ alkyl-C₁-C₆ alkoxy, C₁-C₆ alkyl-NR¹—C₁-C₆ alkyl, NR³R⁴, —C(O)NR³R⁴, —S(O)C₁-C₆ alkyl, —S(O)₂NR³R⁴, and —S(O)₂C₁-C₆ alkyl; and all other variables are as defined herein.

In some embodiments, the compound is of Formula (A-1-b-I-C-3-a-II):

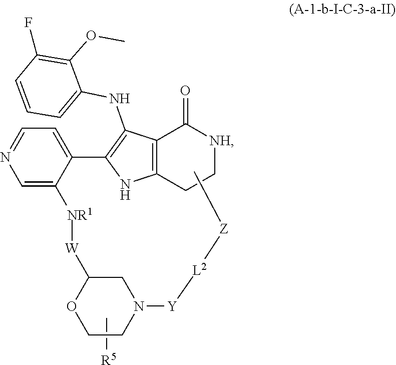

or a pharmaceutically acceptable salt, prodrug, stereoisomer, solvate, or tautomer thereof, wherein W is (CH₂)_{w}; w is an integer selected from 0, 1, 2, 3, 4, 5, 6, 7, 8, 9, and 10; R⁵ is selected from H, halogen, OH, oxo, CN, C₁-C₆ alkyl, C₁-C₆ alkoxy, C₁-C₆ alkyl-C₁-C₆ alkoxy, C₁-C₆ alkyl-NR¹—C₁-C₆ alkyl, NR³R⁴, —C(O)NR³R⁴, —S(O)C₁-C₆ alkyl, —S(O)₂NR³R⁴, and —S(O)₂C₁-C₆ alkyl; and all other variables are as defined herein.

In some embodiments, the compound is of Formula (A-1-b-I-D):

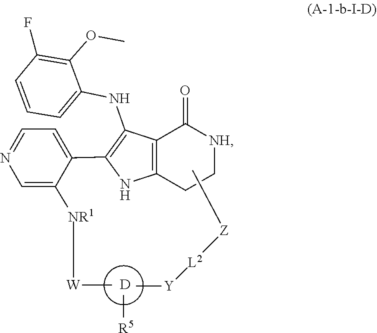

or a pharmaceutically acceptable salt, prodrug, stereoisomer, solvate, or tautomer thereof, wherein Ring D is 5-10 membered monocyclic or bicyclic heteroaryl comprising 1-3 heteroatoms selected from N, O, S; W is (CH₂)_{w}; w is an integer selected from 0, 1, 2, 3, 4, 5, 6, 7, 8, 9, and 10; R⁵ is selected from H, halogen, OH, oxo, CN, C₁-C₆ alkyl, C₁-C₆ alkoxy, C₁-C₆ alkyl-C₁-C₆ alkoxy, C₁-C₆ alkyl-NR¹—C₁-C₆ alkyl, NR³R⁴, —C(O)NR³R⁴, —S(O)C₁-C₆ alkyl, —S(O)₂NR³R⁴, and —S(O)₂C₁-C₆ alkyl and all other variables are as defined herein.

In some embodiments, the compound is of Formula (A-1-b-I-D-1):

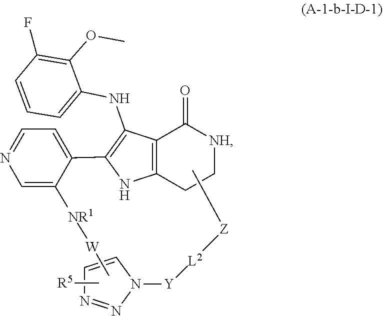

or a pharmaceutically acceptable salt, prodrug, stereoisomer, solvate, or tautomer thereof, wherein W is (CH₂)_{w}; w is an integer selected from 0, 1, 2, 3, 4, 5, 6, 7, 8, 9, and 10; R⁵ is selected from H, halogen, OH, oxo, CN, C₁-C₆ alkyl, C₁-C₆ alkoxy, C₁-C₆ alkyl-C₁-C₆ alkoxy, C₁-C₆ alkyl-NR¹—C₁-C₆ alkyl, NR³R⁴, —C(O)NR³R⁴, —S(O)C₁-C₆ alkyl, —S(O)₂NR³R⁴, and —S(O)₂C₁-C₆ alkyl and all other variables are as defined herein.

In some embodiments, the compound is of Formula (A-1-b-I-D-1-a):

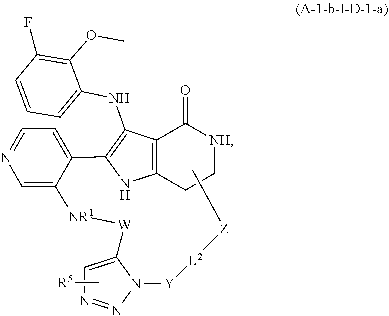

or a pharmaceutically acceptable salt, prodrug, stereoisomer, solvate, or tautomer thereof, wherein W is (CH₂)_{w}; w is an integer selected from 0, 1, 2, 3, 4, 5, 6, 7, 8, 9, and 10; R⁵ is selected from H, halogen, OH, oxo, CN, C₁-C₆ alkyl, C₁-C₆ alkoxy, C₁-C₆ alkyl-C₁-C₆ alkoxy, C₁-C₆ alkyl-NR¹—C₁-C₆ alkyl, NR³R⁴, —C(O)NR³R⁴, —S(O)C₁-C₆ alkyl, —S(O)₂NR³R⁴, and —S(O)₂C₁-C₆ alkyl and all other variables are as defined herein.

In some embodiments, the compound is of Formula (A-1-b-I-D-1-b):

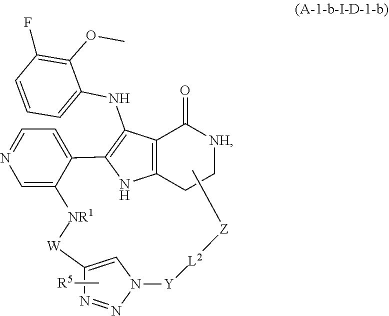

or a pharmaceutically acceptable salt, prodrug, stereoisomer, solvate, or tautomer thereof, wherein W is (CH₂)_{w}; w is an integer selected from 0, 1, 2, 3, 4, 5, 6, 7, 8, 9, and 10; R⁵ is selected from H, halogen, OH, oxo, CN, C₁-C₆ alkyl, C₁-C₆ alkoxy, C₁-C₆ alkyl-C₁-C₆ alkoxy, C₁-C₆ alkyl-NR¹—C₁-C₆ alkyl, NR³R⁴, —C(O)NR³R⁴, —S(O)C₁-C₆ alkyl, —S(O)₂NR³R⁴, and —S(O)₂C₁-C₆ alkyl and all other variables are as defined herein.

In some embodiments, the compound is of Formula (A-B):

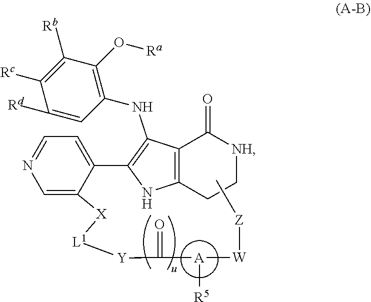

or a pharmaceutically acceptable salt, prodrug, stereoisomer, solvate, or tautomer thereof, wherein Ring A is selected from C₃-C₁₀-cycloalkane, arene, heterocycle, heteroarene; W is (CH₂)_{w}; w is an integer selected from 0, 1, 2, 3, 4, 5, 6, 7, 8, 9, and 10; R⁵ is selected from H, halogen, OH, oxo, CN, C₁-C₆ alkyl, C₁-C₆ alkoxy, C₁-C₆ alkyl-C₁-C₆ alkoxy, C₁-C₆ alkyl-NR¹—C₁-C₆ alkyl, NR³R⁴, —C(O)NR³R⁴, —S(O)C₁-C₆ alkyl, —S(O)₂NR³R⁴, and —S(O)₂C₁-C₆ alkyl; u is an integer selected from 0 and 1; and all other variables are as defined herein.

In more specific aspect, the present disclosure provides compounds of Formula (I) and salts, stereoisomers, solvates, prodrugs, isotopic derivatives, and tautomers thereof:

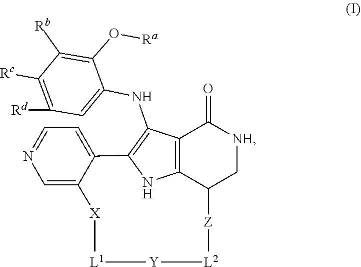

Wherein R^{a}, R^{b}, R^{c}, R^{d}, X, Li, Y, L², and Z are as described herein.

It is understood that, for a compound of Formula (I), R^{a}, R^{b}, R^{c}, R^{d}, X, Li, Y, L², and Z can each be, where applicable, selected from the groups described herein, and any group described herein for any R^{a}, R^{b}, R^{c}, R^{d}, X, Li, Y, L², and Z can be combined, where applicable, with any group described herein for one or more of the remainder of R^{a}, R^{b}, R^{c}, R^{d}, X, Li, Y, L², and Z.

In some embodiments, the compound is of Formula (I)

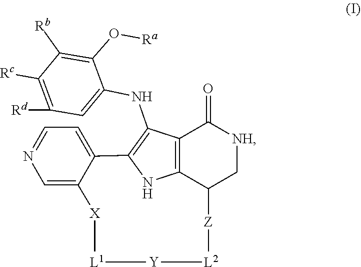

wherein
R^{a} is selected from H, C₁-C₆ alkyl, C₂-C₆ alkenyl, C₂-C₆ alkynyl, C₃-C₁₀ cycloalkyl, heterocyclyl, aryl, and heteroaryl, wherein the alkyl, alkenyl, alkynyl, cycloalkyl, heterocyclyl, aryl, or heteroaryl is optionally substituted with one or more substituents independently selected from halogen, OH, ═O, NR³R⁴, CN, NO₂, COOR¹, CONR³R⁴;
R^{b}, R^{c}, R^{d} are each independently selected from H, halogen, OH, NH₂, CN, NO₂, C₁-C₆ alkyl, and C₁-C₆ alkoxy, wherein alkyl or alkoxy is optionally substituted with one or more halogen, CN;
X is selected from a bond, O, NR¹, C(R²)₂, S, S(O), S(O)₂, C(O), C(O)O, C(O)NR¹, OC(O)O, N(R²)C(O)NR², arenediyl;
R¹ is selected from H, C₁-C₆ alkyl, C₃-C₁₀ cycloalkyl, C₂-C₆ alkenyl, C₂-C₆ alkynyl, C₁-C₆ acyl, heterocycle, aryl, heteroaryl, and S(O)₂R² wherein the alkyl, alkenyl, alkynyl, heterocycle, cycloalkyl, aryl, or heteroaryl is optionally substituted with one or more substituents independently selected from halogen, OH, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, heteroaryl;
each R² is independently selected from H, C₁-C₆ alkyl, C₃-C₁₀ cycloalkyl, C₂-C₆ alkenyl, C₂-C₆ alkynyl, heterocycle, aryl, and heteroaryl wherein the alkyl, alkenyl, alkynyl, heterocycle, cycloalkyl, aryl, or heteroaryl is optionally substituted with one or more substituents independently selected from halogen, OH, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, heteroaryl;
Y is selected from a bond, O, NR¹, C(R²)₂, S, S(O), S(O)₂, C(O), C(O)O, C(O)NR¹, OC(O)O, N(R²)C(O)NR²;
Z is selected from a bond, O, C(R²)₂;
L¹ is selected from heterocyclediyl-C₁-C₁₀-alkanediyl, C₁-C₁₀ alkanediyl, C₂-C₁₀ alkenediyl, C₂-C₁₀ alkynediyl, C₃-C₁₀-cycloalkanediyl, C₃-C₁₀-cycloalkanediyl-C₁-C₁₀-alkanediyl, arenediyl, arenediyl-C₁-C₁₀-alkanediyl, heterocyclediyl, heterocyclediyl-C(O), heteroarenediyl, heteroarenediyl-C₁-C₁₀ alkanediyl, C₁-C₆ alkanediyl-O—C₁-C₆ alkanediyl, C₁-C₆ alkanediyl-NR¹-C₁-C₆ alkanediyl, C₁-C₆ alkanediyl-C(O)NR¹—C₁-C₆ alkanediyl, wherein alkanediyl, alkendiyl, alkynediyl, arenediyl, heterocyclediyl or heteroarenediyl heteroaryl is optionally substituted with one or more substituents independently selected from halogen, OH, oxo, CN, C₁-C₆ alkyl, C₁-C₆ alkoxy, C₁-C₆ alkyl-C₁-C₆ alkoxy, C₁-C₆ alkyl-NR¹—C₁-C₆ alkyl, NR³R⁴, —C(O)NR³R⁴, —S(O)C₁-C₆ alkyl, —S(O)₂NR³R⁴, aryl, and —S(O)₂C₁-C₆ alkyl;
L² is selected from C₁-C₁₀ alkanediyl, C₂-C₁₀ alkenediyl, C₂-C₁₀ alkynediyl, C₃-C₁₀-cycloalkanediyl, C₃-C₁₀-cycloalkanediyl-C₁-C₁₀-alkanediyl, arenediyl, arenediyl-C₁-C₁₀-alkanediyl, heterocyclediyl, heterocyclediyl-C₁-C₁₀-alkanediyl, heterocyclediyl-C(O), heteroarenediyl, heteroarenediyl-C₁-C₁₀ alkanediyl, C₁-C₆ alkanediyl-O—C₁-C₆ alkanediyl, C₁-C₆ alkanediyl-NR¹—C₁-C₆ alkanediyl, C₁-C₆ alkanediyl-C(O)NR¹—C₁-C₆ alkanediyl, wherein alkanediyl, alkendiyl, alkynediyl, arenediyl, heterocyclediyl or heteroarenediyl heteroaryl is optionally substituted with one or more substituents independently selected from halogen, OH, oxo, CN, C₁-C₆ alkyl, C₁-C₆ alkoxy, C₁-C₆ alkyl-C₁-C₆ alkoxy, C₁-C₆ alkyl-NR¹—C₁-C₆ alkyl, NR³R⁴, —C(O)NR³R⁴, —S(O)C₁-C₆ alkyl, —S(O)₂NR³R⁴, aryl, and —S(O)₂C₁-C₆ alkyl;
R³ and R⁴ are each independently selected from H, C₁-C₆ alkyl, C₁-C₆ acyl and C₃-C₁₀ cycloalkyl;
or R³ and R⁴ together with the atoms to which they are attached and any intervening atoms, form a 5-14 membered heterocycle, wherein the heterocycle is optionally substituted with one or more substituents independently selected from halogen, OH, ═O, CN, NO₂, C₁-C₆ alkyl, C₁-C₆ alkoxy.

In another more specific aspect, the present disclosure provides compounds of Formula (II) and salts, stereoisomers, solvates, prodrugs, isotopic derivatives, and tautomers thereof.

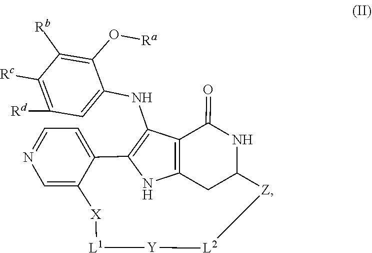

Wherein R^{a}, R^{b}, R^{c}, R^{d}, X, Li, Y, L², and Z are as described herein.

It is understood that, for a compound of Formula (I), R^{a}, R^{b}, R^{c}, R^{d}, X, Li, Y, L², and Z can each be, where applicable, selected from the groups described herein, and any group described herein for any R^{a}, R^{b}, R^{c}, R^{d}, X, Li, Y, L², and Z can be combined, where applicable, with any group described herein for one or more of the remainder of R^{a}, R^{b}, R^{c}, R^{d}, X, L¹, Y, L², and Z.

In some embodiments, the compound is of Formula (II)

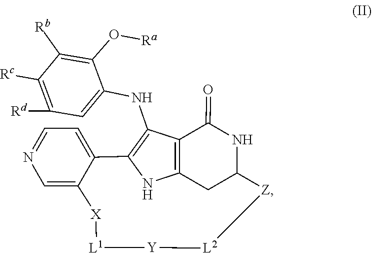

wherein
R^{a} is selected from H, C₁-C₆ alkyl, C₂-C₆ alkenyl, C₂-C₆ alkynyl, C₃-C₁₀ cycloalkyl, heterocyclyl, aryl, and heteroaryl, wherein the alkyl, alkenyl, alkynyl, cycloalkyl, heterocyclyl, aryl, or heteroaryl is optionally substituted with one or more substituents independently selected from halogen, OH, ═O, NR³R⁴, CN, NO₂, COOR¹, CONR³R⁴;
R^{b}, R^{c}, R^{d} are each independently selected from H, halogen, OH, NH₂, CN, NO₂, C₁-C₆ alkyl, and C₁-C₆ alkoxy, wherein alkyl or alkoxy is optionally substituted with one or more halogen, CN;
X is selected from a bond, O, NR¹, C(R²)₂, S, S(O), S(O)₂, C(O), C(O)O, C(O)NR¹, OC(O)O, N(R²)C(O)NR², arenediyl;
R¹ is selected from H, C₁-C₆ alkyl, C₃-C₁₀ cycloalkyl, C₂-C₆ alkenyl, C₂-C₆ alkynyl, C₁-C₆ acyl, heterocycle, aryl, heteroaryl, and S(O)₂R² wherein the alkyl, alkenyl, alkynyl, heterocycle, cycloalkyl, aryl, or heteroaryl is optionally substituted with one or more substituents independently selected from halogen, OH, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, heteroaryl;
each R² is independently selected from H, C₁-C₆ alkyl, C₃-C₁₀ cycloalkyl, C₂-C₆ alkenyl, C₂-C₆ alkynyl, heterocycle, aryl, and heteroaryl wherein the alkyl, alkenyl, alkynyl, heterocycle, cycloalkyl, aryl, or heteroaryl is optionally substituted with one or more substituents independently selected from halogen, OH, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, heteroaryl;
Y is selected from a bond, O, NR¹, C(R²)₂, S, S(O), S(O)₂, C(O), C(O)O, C(O)NR¹, OC(O)O, N(R²)C(O)NR²;
Z is selected from a bond, O, C(R²)₂;
L¹ is selected from heterocyclediyl-C₁-C₁₀-alkanediyl, C₁-C₁₀ alkanediyl, C₂-C₁₀ alkenediyl, C₂-C₁₀ alkynediyl, C₃-C₁₀-cycloalkanediyl, C₃-C₁₀-cycloalkanediyl-C₁-C₁₀-alkanediyl, arenediyl, arenediyl-C₁-C₁₀-alkanediyl, heterocyclediyl, heterocyclediyl-C(O), heteroarenediyl, heteroarenediyl-C₁-C₁₀ alkanediyl, C₁-C₆ alkanediyl-O—C₁-C₆ alkanediyl, C₁-C₆ alkanediyl-NR¹—C₁-C₆ alkanediyl, C₁-C₆ alkanediyl-C(O)NR¹—C₁-C₆ alkanediyl, wherein alkanediyl, alkendiyl, alkynediyl, arenediyl, heterocyclediyl or heteroarenediyl heteroaryl is optionally substituted with one or more substituents independently selected from halogen, OH, oxo, CN, C₁-C₆ alkyl, C₁-C₆ alkoxy, C₁-C₆ alkyl-C₁-C₆ alkoxy, C₁-C₆ alkyl-NR¹—C₁-C₆ alkyl, NR³R⁴, —C(O)NR³R⁴, —S(O)C₁-C₆ alkyl, —S(O)₂NR³R⁴, aryl, and —S(O)₂C₁-C₆ alkyl;
L² is selected from C₁-C₁₀ alkanediyl, C₂-C₁₀ alkenediyl, C₂-C₁₀ alkynediyl, C₃-C₁₀-cycloalkanediyl, C₃-C₁₀-cycloalkanediyl-C₁-C₁₀-alkanediyl, arenediyl, arenediyl-C₁-C₁₀-alkanediyl, heterocyclediyl, heterocyclediyl-C₁-C₁₀-alkanediyl, heterocyclediyl-C(O), heteroarenediyl, heteroarenediyl-C₁-C₁₀ alkanediyl, C₁-C₆ alkanediyl-O—C₁-C₆ alkanediyl, C₁-C₆ alkanediyl-NR¹—C₁-C₆ alkanediyl, C₁-C₆ alkanediyl-C(O)NR¹—C₁-C₆ alkanediyl, wherein alkanediyl, alkendiyl, alkynediyl, arenediyl, heterocyclediyl or heteroarenediyl heteroaryl is optionally substituted with one or more substituents independently selected from halogen, OH, oxo, CN, C₁-C₆ alkyl, C₁-C₆ alkoxy, C₁-C₆ alkyl-C₁-C₆ alkoxy, C₁-C₆ alkyl-NR¹—C₁-C₆ alkyl, NR³R⁴, —C(O)NR³R⁴, —S(O)C₁-C₆ alkyl, —S(O)₂NR³R⁴, aryl, and —S(O)₂C₁-C₆ alkyl; R³ and R⁴ are each independently selected from H, C₁-C₆ alkyl, C₁-C₆ acyl and C₃-C₁₀ cycloalkyl;
or R³ and R⁴ together with the atoms to which they are attached and any intervening atoms, form a 5-14 membered heterocycle, wherein the heterocycle is optionally substituted with one or more substituents independently selected from halogen, OH, ═O, CN, NO₂, C₁-C₆ alkyl, C₁-C₆ alkoxy.

In some embodiments, R^{a} is selected from H, C₁-C₆ alkyl, C₂-C₆ alkenyl, C₂-C₆ alkynyl, C₃-C₁₀ cycloalkyl, heterocyclyl, aryl, and heteroaryl, wherein the alkyl, alkenyl, alkynyl, cycloalkyl, heterocyclyl, aryl, or heteroaryl is optionally substituted with one or more substituents independently selected from halogen, OH, ═O, NR³R⁴, CN, NO₂, COOR¹, CONR³R⁴.

In some embodiments, R^{a} is H.

In some embodiments, R^{a} is C₁-C₆ alkyl.

In some embodiments, R^{a} is C₁-C₆ alkyl substituted with one or more halogen.

In some embodiments, R^{a} is C₁-C₆ alkyl substituted with one or more F.

In some embodiments, R^{a} is —CH₃.

In some embodiments, R^{a} is —CH₂CH₃.

In some embodiments, R^{a} is —CH₂F.

In some embodiments, R^{a} is —CHF₂.

In some embodiments, R^{a} is —CF₃.

In some embodiments, R^{a} is —CH₂CHF₂.

In some embodiments, R^{a} is C₂-C₆ alkenyl.

In some embodiments, R^{a} is —CH₂CH═CH₂.

In some embodiments, R^{b} is selected from H, halogen, OH, NH₂, CN, NO₂, C₁-C₆ alkyl, and C₁-C₆ alkoxy, wherein alkyl or alkoxy is optionally substituted with one or more halogen, CN.

In some embodiments, R^{b} is H.

In some embodiments, R^{b} is halogen.

In some embodiments, R^{b} is F.

In some embodiments, R^{b} is C₁.

In some embodiments, R^{b} is Br.

In some embodiments, R^{b} is OH.

In some embodiments, R^{b} is CN.

In some embodiments, R^{b} is CF₃.

In some embodiments, R^{c} is selected from H, halogen, OH, NH₂, CN, NO₂, C₁-C₆ alkyl, and C₁-C₆ alkoxy, wherein alkyl or alkoxy is optionally substituted with one or more halogen, CN.

In some embodiments, R^{c} is H.

In some embodiments, R^{c} is halogen.

In some embodiments, R^{c} is F.

In some embodiments, R^{c} is C₁.

In some embodiments, R^{c} is Br.

In some embodiments, R^{c} is OH.

In some embodiments, R^{c} is CN.

In some embodiments, R^{c} is CF₃.

In some embodiments, R^{d} is selected from H, halogen, OH, NH₂, CN, NO₂, C₁-C₆ alkyl, and C₁-C₆ alkoxy, wherein alkyl or alkoxy is optionally substituted with one or more halogen, CN.

In some embodiments, R^{d} is H.

In some embodiments, R^{d} is halogen.

In some embodiments, R^{d} is F.

In some embodiments, R^{d} is C₁.

In some embodiments, R^{d} is Br.

In some embodiments, R^{d} is OH.

In some embodiments, R^{d} is CN.

In some embodiments, R^{d} is CF₃.

In some embodiments, the Compound is of Formula (I-A)

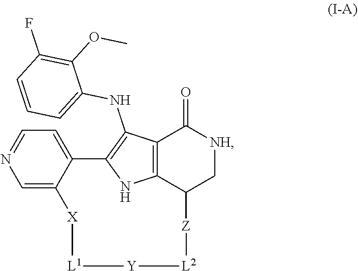

wherein
X is selected from O, a bond, NR¹, C(R²)₂, S, S(O), S(O)₂, C(O), C(O)O, C(O)NR¹, OC(O)O, N(R²)C(O)NR², arenediyl;
R¹ is selected from H, C₁-C₆ alkyl, C₃-C₁₀ cycloalkyl, C₂-C₆ alkenyl, C₂-C₆ alkynyl, C₁-C₆ acyl, heterocycle, aryl, heteroaryl, and S(O)₂R² wherein the alkyl, alkenyl, alkynyl, heterocycle, cycloalkyl, aryl, or heteroaryl is optionally substituted with one or more substituents independently selected from halogen, OH, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, heteroaryl;
each R² is independently selected from H, C₁-C₆ alkyl, C₃-C₁₀ cycloalkyl, C₂-C₆ alkenyl, C₂-C₆ alkynyl, heterocycle, aryl, and heteroaryl wherein the alkyl, alkenyl, alkynyl, heterocycle, cycloalkyl, aryl, or heteroaryl is optionally substituted with one or more substituents independently selected from halogen, OH, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, heteroaryl;
Y is selected from a bond, O, NR¹, C(R²)₂, S, S(O), S(O)₂, C(O), C(O)O, C(O)NR¹, OC(O)O, N(R²)C(O)NR²;
Z is selected from a bond, O, C(R²)₂;
L¹ is selected from heterocyclediyl-C₁-C₁₀-alkanediyl, C₁-C₁₀ alkanediyl, C₂-C₁₀ alkenediyl, C₂-C₁₀ alkynediyl, C₃-C₁₀-cycloalkanediyl, C₃-C₁₀-cycloalkanediyl-C₁-C₁₀-alkanediyl, arenediyl, arenediyl-C₁-C₁₀-alkanediyl, heterocyclediyl, heterocyclediyl-C(O), heteroarenediyl, heteroarenediyl-C₁-C₁₀ alkanediyl, C₁-C₆ alkanediyl-O—C₁-C₆ alkanediyl, C₁-C₆ alkanediyl-NR¹—C₁-C₆ alkanediyl, C₁-C₆ alkanediyl-C(O)NR¹—C₁-C₆ alkanediyl, wherein alkanediyl, alkendiyl, alkynediyl, arenediyl, heterocyclediyl or heteroarenediyl heteroaryl is optionally substituted with one or more substituents independently selected from halogen, OH, oxo, CN, C₁-C₆ alkyl, C₁-C₆ alkoxy, C₁-C₆ alkyl-C₁-C₆ alkoxy, C₁-C₆ alkyl-NR¹—C₁-C₆ alkyl, NR³R⁴, —C(O)NR³R⁴, —S(O)C₁-C₆ alkyl, —S(O)₂NR³R⁴, aryl, and —S(O)₂C₁-C₆ alkyl;
L² is selected from C₁-C₁₀ alkanediyl, C₂-C₁₀ alkenediyl, C₂-C₁₀ alkynediyl, C₃-C₁₀-cycloalkanediyl, C₃-C₁₀-cycloalkanediyl-C₁-C₁₀-alkanediyl, arenediyl, arenediyl-C₁-C₁₀-alkanediyl, heterocyclediyl, heterocyclediyl-C₁-C₁₀-alkanediyl, heterocyclediyl-C(O), heteroarenediyl, heteroarenediyl-C₁-C₁₀ alkanediyl, C₁-C₆ alkanediyl-O—C₁-C₆ alkanediyl, C₁-C₆ alkanediyl-NR¹—C₁-C₆ alkanediyl, C₁-C₆ alkanediyl-C(O)NR¹—C₁-C₆ alkanediyl, wherein alkanediyl, alkendiyl, alkynediyl, arenediyl, heterocyclediyl or heteroarenediyl heteroaryl is optionally substituted with one or more substituents independently selected from halogen, OH, oxo, CN, C₁-C₆ alkyl, C₁-C₆ alkoxy, C₁-C₆ alkyl-C₁-C₆ alkoxy, C₁-C₆ alkyl-NR¹—C₁-C₆ alkyl, NR³R⁴, —C(O)NR³R⁴, —S(O)C₁-C₆ alkyl, —S(O)₂NR³R⁴, aryl, and —S(O)₂C₁-C₆ alkyl;
R³ and R⁴ are each independently selected from H, C₁-C₆ alkyl, C₁-C₆ acyl and C₃-C₁₀ cycloalkyl;
or R³ and R⁴ together with the atoms to which they are attached and any intervening atoms, form a 5-14 membered heterocycle, wherein the heterocycle is optionally substituted with one or more substituents independently selected from halogen, OH, ═O, CN, NO₂, C₁-C₆ alkyl, C₁-C₆ alkoxy.

In some embodiments, the Compound is of Formula (II-A)

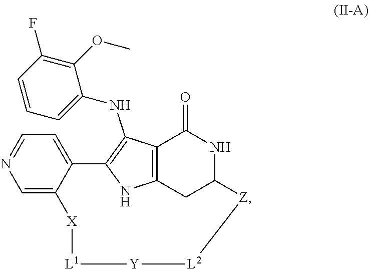

wherein
X is selected from O, a bond, NR¹, C(R²)₂, S, S(O), S(O)₂, C(O), C(O)O, C(O)NR¹, OC(O)O, N(R²)C(O)NR², arenediyl;
R¹ is selected from H, C₁-C₆ alkyl, C₃-C₁₀ cycloalkyl, C₂-C₆ alkenyl, C₂-C₆ alkynyl, C₁-C₆ acyl, heterocycle, aryl, heteroaryl, and S(O)₂R² wherein the alkyl, alkenyl, alkynyl, heterocycle, cycloalkyl, aryl, or heteroaryl is optionally substituted with one or more substituents independently selected from halogen, OH, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, heteroaryl;
each R² is independently selected from H, C₁-C₆ alkyl, C₃-C₁₀ cycloalkyl, C₂-C₆ alkenyl, C₂-C₆ alkynyl, heterocycle, aryl, and heteroaryl wherein the alkyl, alkenyl, alkynyl, heterocycle, cycloalkyl, aryl, or heteroaryl is optionally substituted with one or more substituents independently selected from halogen, OH, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, heteroaryl;
Y is selected from a bond, O, NR¹, C(R²)₂, S, S(O), S(O)₂, C(O), C(O)O, C(O)NR¹, OC(O)O, N(R²)C(O)NR²;
Z is selected from a bond, O, C(R²)₂;
L¹ is selected from heterocyclediyl-C₁-C₁₀-alkanediyl, C₁-C₁₀ alkanediyl, C₂-C₁₀ alkenediyl, C₂-C₁₀ alkynediyl, C₃-C₁₀-cycloalkanediyl, C₃-C₁₀-cycloalkanediyl-C₁-C₁₀-alkanediyl, arenediyl, arenediyl-C₁-C₁₀-alkanediyl, heterocyclediyl, heterocyclediyl-C(O), heteroarenediyl, heteroarenediyl-C₁-C₁₀ alkanediyl, C₁-C₆ alkanediyl-O—C₁-C₆ alkanediyl, C₁-C₆ alkanediyl-NR¹—C₁-C₆ alkanediyl, C₁-C₆ alkanediyl-C(O)NR¹—C₁-C₆ alkanediyl, wherein alkanediyl, alkendiyl, alkynediyl, arenediyl, heterocyclediyl or heteroarenediyl heteroaryl is optionally substituted with one or more substituents independently selected from halogen, OH, oxo, CN, C₁-C₆ alkyl, C₁-C₆ alkoxy, C₁-C₆ alkyl-C₁-C₆ alkoxy, C₁-C₆ alkyl-NR¹—C₁-C₆ alkyl, NR³R⁴, —C(O)NR³R⁴, —S(O)C₁-C₆ alkyl, —S(O)₂NR³R⁴, aryl, and —S(O)₂C₁-C₆ alkyl;
L² is selected from C₁-C₁₀ alkanediyl, C₂-C₁₀ alkenediyl, C₂-C₁₀ alkynediyl, C₃-C₁₀-cycloalkanediyl, C₃-C₁₀-cycloalkanediyl-C₁-C₁₀-alkanediyl, arenediyl, arenediyl-C₁-C₁₀-alkanediyl, heterocyclediyl, heterocyclediyl-C₁-C₁₀-alkanediyl, heterocyclediyl-C(O), heteroarenediyl, heteroarenediyl-C₁-C₁₀ alkanediyl, C₁-C₆ alkanediyl-O—C₁-C₆ alkanediyl, C₁-C₆ alkanediyl-NR¹—C₁-C₆ alkanediyl, C₁-C₆ alkanediyl-C(O)NR¹—C₁-C₆ alkanediyl, wherein alkanediyl, alkendiyl, alkynediyl, arenediyl, heterocyclediyl or heteroarenediyl heteroaryl is optionally substituted with one or more substituents independently selected from halogen, OH, oxo, CN, C₁-C₆ alkyl, C₁-C₆ alkoxy, C₁-C₆ alkyl-C₁-C₆ alkoxy, C₁-C₆ alkyl-NR¹—C₁-C₆ alkyl, NR³R⁴, —C(O)NR³R⁴, —S(O)C₁-C₆ alkyl, —S(O)₂NR³R⁴, aryl, and —S(O)₂C₁-C₆ alkyl;
R³ and R⁴ are each independently selected from H, C₁-C₆ alkyl, C₁-C₆ acyl and C₃-C₁₀ cycloalkyl;
or R³ and R⁴ together with the atoms to which they are attached and any intervening atoms, form a 5-14 membered heterocycle, wherein the heterocycle is optionally substituted with one or more substituents independently selected from halogen, OH, ═O, CN, NO₂, C₁-C₆ alkyl, C₁-C₆ alkoxy.

In some embodiments, X is selected from a bond, O, NR¹, C(R²)₂, S, S(O), S(O)₂, C(O), C(O)O, C(O)NR¹, OC(O)O, or N(R²)C(O)NR².

In some embodiments, X is a bond.

In some embodiments, X is a single bond.

In some embodiments, X is —O—.

In some embodiments, X is

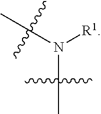

In some embodiments, X is

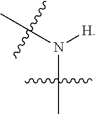

In some embodiments, X is

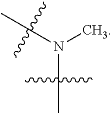 [SMILES: *N(*)C]

In some embodiments, X is

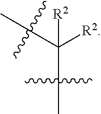

In some embodiments, X is

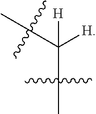

In some embodiments, X is

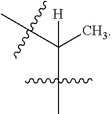 [SMILES: *C(*)C]

In some embodiments, X is

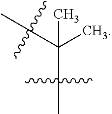 [SMILES: *C(*)(C)C]

In some embodiments, X is

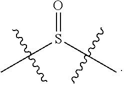 [SMILES: *S(*)=O]

In some embodiments, X is

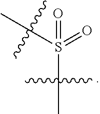 [SMILES: *S(*)(=O)=O]

In some embodiments, X is

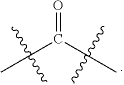 [SMILES: *C(*)=O]

In some embodiments, X is

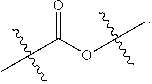 [SMILES: *OC(*)=O]

In some embodiments, X is

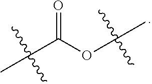 [SMILES: *OC(*)=O]

In some embodiments, X is

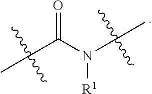

In some embodiments, X is

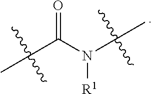

In some embodiments, X is

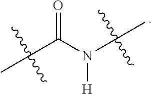 [SMILES: *NC(*)=O]

In some embodiments, X is

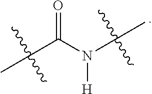 [SMILES: *NC(*)=O]

In some embodiments, X is

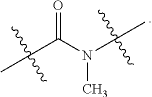 [SMILES: *C(=O)N(*)C]

In some embodiments, X is

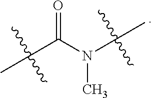 [SMILES: *C(=O)N(*)C]

In some embodiments, R¹ is selected from H, C₁-C₆ alkyl, C₃-C₁₀ cycloalkyl, C₂-C₆ alkenyl, C₂-C₆ alkynyl, C₁-C₆ acyl, heterocycle, aryl, heteroaryl, or S(O)₂R² wherein the alkyl, alkenyl, alkynyl, heterocycle, cycloalkyl, aryl, or heteroaryl is optionally substituted with one or more substituents independently selected from halogen, OH, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, heteroaryl.

In some embodiments, R¹ is H.

In some embodiments, R¹ is CH₃.

In some embodiments, R² is selected from H, C₁-C₆ alkyl, C₃-C₁₀ cycloalkyl, C₂-C₆ alkenyl, C₂-C₆ alkynyl, heterocycle, aryl, or heteroaryl wherein the alkyl, alkenyl, alkynyl, heterocycle, cycloalkyl, aryl, or heteroaryl is optionally substituted with one or more substituents independently selected from halogen, OH, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, heteroaryl.

In some embodiments, R² is H.

In some embodiments, R² is CH₃.

In some embodiments, Y is a bond, O, NR¹, C(R²)₂, S, S(O), S(O)₂; C(O); C(O)O, C(O)NR¹, OC(O)O, N(R²)C(O)NR², arenediyl.

In some embodiments, Y is a bond.

In some embodiments, Y is a single bond.

In some embodiments, Y is —O—.

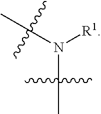

In some embodiments, Y is

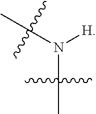

In some embodiments, Y is

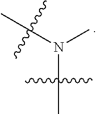 [SMILES: *N(*)C]

In some embodiments, Y is

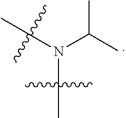 [SMILES: *N(*)C(C)C]

In some embodiments, Y is

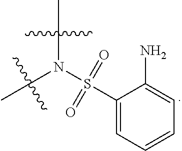 [SMILES: *N(*)S(=O)(=O)c1ccccc1N]

In some embodiments, Y is

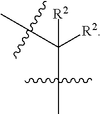

In some embodiments, Y is

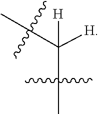

In some embodiments, Y is

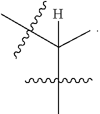 [SMILES: *C(*)C]

In some embodiments, Y is

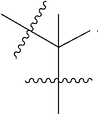 [SMILES: *C(*)(C)C]

In some embodiments, Y is —S—.

In some embodiments, Y is

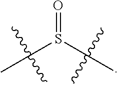

In some embodiments, Y is

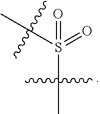 [SMILES: *S(*)(=O)=O]

In some embodiments, Y is

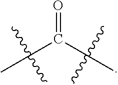

In some embodiments, Y is

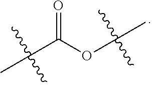 [SMILES: *OC(*)=O]

In some embodiments, Y is

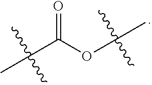 [SMILES: *OC(*)=O]

In some embodiments, Y is

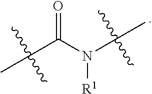

In some embodiments, Y is

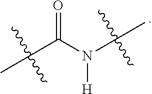 [SMILES: *NC(*)=O]

In some embodiments, Y is

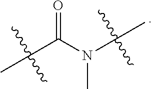 [SMILES: *C(=O)N(*)C]

In some embodiments, Y is —OC(O)O—.

In some embodiments, Y is N(R²)C(O)NR².

In some embodiments, Y is

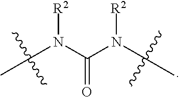

In some embodiments, Y is

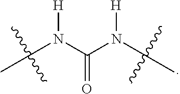 [SMILES: *NC(=O)N*]

In some embodiments, Y is

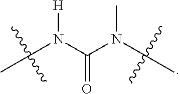 [SMILES: *NC(=O)N(*)C]

In some embodiments, Y is

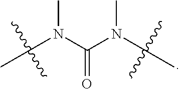 [SMILES: *N(C)C(=O)N(*)C]

In some embodiments, Z is selected from a bond, O, or C(R²)₂.

In some embodiments, Z is a bond.

In some embodiments, Z is a single bond.

In some embodiments, Z is —O—.

In some embodiments, Z is C(R²)₂.

In some embodiments, Z is CH₂.

In some embodiments, Li is selected from C₁-C₁₀ alkanediyl, C₂-C₁₀ alkenediyl, C₂-C₁₀ alkynediyl, C₃-C₁₀-cycloalkanediyl, C₃-C₁₀-cycloalkanediyl-C₁-C₁₀-alkanediyl, arenediyl, arenediyl-C₁-C₁₀-alkanediyl, heterocyclediyl, heterocyclediyl-C₁-C₁₀-alkanediyl, heterocyclediyl-C(O), heteroarenediyl, heteroarenediyl-C₁-C₁₀ alkanediyl, C₁-C₆ alkanediyl-O—C₁-C₆ alkanediyl, C₁-C₆ alkanediyl-NR¹—C₁-C₆ alkanediyl, C₁-C₆ alkanediyl-C(O)NR¹—C₁-C₆ alkanediyl, wherein alkanediyl, alkendiyl, alkynediyl, arenediyl, heterocyclediyl or heteroarenediyl heteroaryl is optionally substituted with one or more substituents independently selected from halogen, OH, oxo, CN, C₁-C₆ alkyl, C₁-C₆ alkoxy, C₁-C₆ alkyl-C₁-C₆ alkoxy, C₁-C₆ alkyl-NR¹—C₁-C₆ alkyl, NR³R⁴, —C(O)NR³R⁴, —S(O)C₁-C₆ alkyl, —S(O)₂NR³R⁴, aryl, and —S(O)₂C₁-C₆ alkyl.

In some embodiments, Li is C₁-C₁₀ alkanediyl.

In some embodiments, Li is C₁-C₁₀ alkanediyl optionally substituted with one or more substituents independently selected from halogen, OH, oxo, CN, C₁-C₆ alkyl, C₁-C₆ alkoxy, C₁-C₆ alkyl-C₁-C₆ alkoxy, C₁-C₆ alkyl-NR¹—C₁-C₆ alkyl, NR³R⁴, —C(O)NR³R⁴, —S(O)C₁-C₆ alkyl, —S(O)₂NR³R⁴, aryl, and —S(O)₂C₁-C₆ alkyl.

In some embodiments, Li is methanediyl.

In some embodiments, Li is ethanediyl.

In some embodiments, Li is 1,1-ethanediyl.

In some embodiments, Li is 1,2-ethanediyl.

In some embodiments, Li is propanediyl.

In some embodiments, Li is 1,3-propanediyl.

In some embodiments, Li is 1-methylpropanediyl-1,3.

In some embodiments, Li is 2-methylpropanediyl-1,3.

In some embodiments, Li is butanediyl.

In some embodiments, Li is 1,4-butanediyl.

In some embodiments, Li is 2-methylbutanediyl-1,4.

In some embodiments, Li is pentanediyl.

In some embodiments, Li is 1,5-pentanediyl.

In some embodiments, Li is 3-methylpentanediyl-1,5.

In some embodiments, Li is 2,3-dimethylpentanediyl-1,5.

In some embodiments, Li is hexanediyl.

In some embodiments, L¹ is 1,6-hexanediyl.

In some embodiments, L¹ is 3-methylhexanediyl-1,6.

In some embodiments, L¹ is 3,4-dimethylhexanediyl-1,6.

In some embodiments, L¹ is C₂-C₁₀ alkenediyl.

In some embodiments, L¹ is E-C₂-C₁₀ alkenediyl.

In some embodiments, L¹ is Z—C₂-C₁₀ alkenediyl.

In some embodiments, L¹ is ethenediyl.

In some embodiments, L¹ is ethenediyl-1,2.

In some embodiments, L¹ is propenediyl.

In some embodiments, L¹ is propenediyl-1,3.

In some embodiments, L¹ is 2-methylpropenediyl-1,3

In some embodiments, L¹ is butenediyl.

In some embodiments, L¹ is butene-1-diyl-1,4.

In some embodiments, L¹ is E-butene-1-diyl-1,4.

In some embodiments, L¹ is Z-butene-1-diyl-1,4.

In some embodiments, L¹ is butene-2-diyl-1,4.

In some embodiments, L¹ is E-butene-2-diyl-1,4.

In some embodiments, L¹ is Z-butene-2-diyl-1,4.

In some embodiments, L¹ is 2-methylbutene-2-diyl-1,4.

In some embodiments, L¹ is E-2-methylbutene-2-diyl-1,4.

In some embodiments, L¹ is Z-2-methylbutene-2-diyl-1,4.

In some embodiments, L¹ is pentenediyl.

In some embodiments, L¹ is pentene-2-diyl-1,5.

In some embodiments, L¹ is E-pentene-2-diyl-1,5.

In some embodiments, L¹ is Z-pentene-2-diyl-1,5.

In some embodiments, L¹ is 2-methylpentene-2-diyl-1,5.

In some embodiments, L¹ is E-2-methylpentene-2-diyl-1,5.

In some embodiments, L¹ is Z-2-methylpentene-2-diyl-1,5.

In some embodiments, L¹ is 3-methylpentene-2-diyl-1,5.

In some embodiments, L¹ is E-3-methylpentene-2-diyl-1,5.

In some embodiments, L¹ is Z-3-methylpentene-2-diyl-1,5.

In some embodiments, L¹ is 2,3-dimethylpenten-2-diyl-1,5.

In some embodiments, L¹ is E-2,3-dimethylpenten-2-diyl-1,5.

In some embodiments, L¹ is Z-2,3-dimethylpenten-2-diyl-1,5.

In some embodiments, L¹ is hexenediyl.

In some embodiments, L¹ is hexene-2-diyl-1,6.

In some embodiments, L¹ is hexene-3-diyl-1,6.

In some embodiments, L¹ is 3-methylhexen-3-diyl-1,6.

In some embodiments, L¹ is E-3-methylhexen-3-diyl-1,6.

In some embodiments, L¹ is Z-3-methylhexen-3-diyl-1,6.

In some embodiments, L¹ is 3,4-dimethylhexen-3-diyl-1,6.

In some embodiments, L¹ is E-3,4-dimethylhexen-3-diyl-1,6.

In some embodiments, L¹ is Z-3,4-dimethylhexen-3-diyl-1,6.

In some embodiments, L¹ is C₂-C₁₀ alkynediyl.

In some embodiments, L¹ is ethynediyl.

In some embodiments, L¹ is propynediyl.

In some embodiments, L¹ is propyne-1-diyl-1,3.

In some embodiments, L¹ is 3-methylpropyne-1-diyl-1,3.

In some embodiments, L¹ is C₃-C₁₀-cycloalkanediyl.

In some embodiments, L¹ is

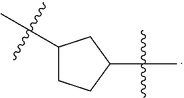 [SMILES: *C1CCC(*)C1]

In some embodiments, L¹ is

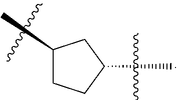

In some embodiments, L¹ is

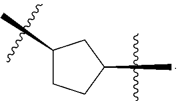

In some embodiments, L¹ is

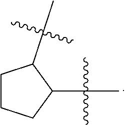 [SMILES: *C1CCCC1*]

In some embodiments, L¹ is

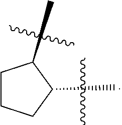

In some embodiments, L¹ is

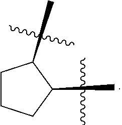

In some embodiments, L¹ is

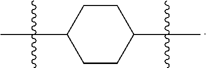 [SMILES: *C1CCC(*)CC1]

In some embodiments, L¹ is

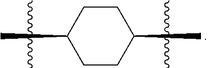

In some embodiments, L¹ is

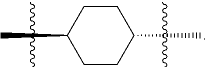

In some embodiments, L¹ is

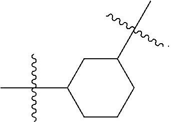 [SMILES: *C1CCCC(*)C1]

In some embodiments, L¹ is

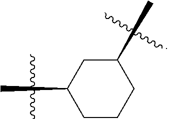

In some embodiments, L¹ is

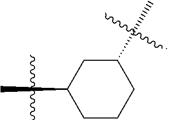

In some embodiments, L¹ is

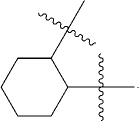 [SMILES: *C1CCCCC1*]

In some embodiments, L¹ is

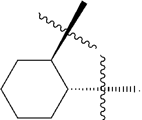

In some embodiments, L¹ is

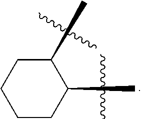

In some embodiments, L¹ is C₃-C₁₀-cycloalkanediyl-C₁-C₁₀-alkanediyl.

In some embodiments, L¹ is

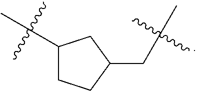 [SMILES: *CC1CCC(*)C1]

In some embodiments, L¹ is

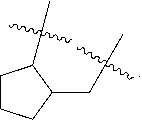 [SMILES: *CC1CCCC1*]

In some embodiments, L¹ is

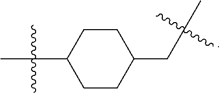 [SMILES: *CC1CCC(*)CC1]

In some embodiments, L¹ is

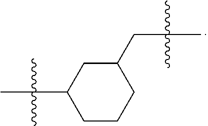 [SMILES: *CC1CCCC(*)C1]

In some embodiments, L¹ is

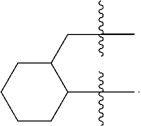 [SMILES: *CC1CCCCC1*]

In some embodiments, L¹ is arenediyl.

In some embodiments, L¹ is

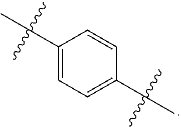 [SMILES: *c1ccc(*)cc1]

In some embodiments, L¹ is

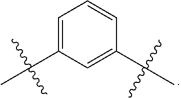 [SMILES: *c1cccc(*)c1]

In some embodiments, L¹ is

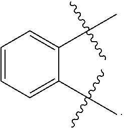 [SMILES: *c1ccccc1*]

In some embodiments, L¹ is arenediyl-C₁-C₁₀-alkanediyl.

In some embodiments, L¹ is arenediyl-methanediyl.

In some embodiments, L¹ is arenediyl-ethanediyl.

In some embodiments, L¹ is arenediyl-1,2-ethanediyl.

In some embodiments, L¹ is arenediyl-propanediyl.

In some embodiments, L¹ is arenediyl-1,3-propanediyl.

In some embodiments, L¹ is arenediyl-butanediyl.

In some embodiments, L¹ is arenediyl-1,4-butanediyl.

In some embodiments, L¹ is arenediyl-pentanediyl.

In some embodiments, L¹ is arenediyl-hexanediyl.

In some embodiments, L¹ is arenediyl-heptanediyl.

In some embodiments, L¹ is arenediyl-octanediyl.

In some embodiments, L¹ is arenediyl-nonanediyl.

In some embodiments, L¹ is arenediyl-decanediyl.

In some embodiments, L¹ is phenylen-C₁-C₁₀-alkanediyl.

In some embodiments, L¹ is

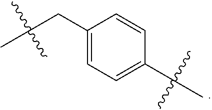 [SMILES: *Cc1ccc(*)cc1]

In some embodiments, L¹

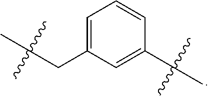 [SMILES: *Cc1cccc(*)c1]

In some embodiments, L₁ is

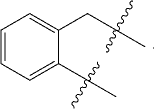 [SMILES: *Cc1ccccc1*]

In some embodiments, L¹ is heterocyclediyl.

In some embodiments, L¹ is 3-membered heterocyclediyl.

In some embodiments, L¹ is 3-membered heterocyclediyl comprising N as a heteroatom.

In some embodiments, L¹ is 4-membered heterocyclediyl.

In some embodiments, L¹ is 4-membered heterocyclediyl comprising N as a heteroatom.

In some embodiments, L¹ is

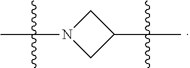 [SMILES: *C1CN(*)C1]

In some embodiments, L¹ is

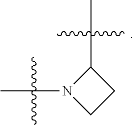 [SMILES: *C1CCN1*]

In some embodiments, L¹ is

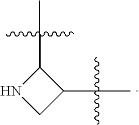 [SMILES: *C1CNC1*]

In some embodiments, L¹ is

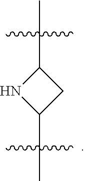 [SMILES: *C1CC(*)N1]

In some embodiments, L¹ is 5-membered heterocyclediyl.

In some embodiments, L¹ is 5-membered heterocyclediyl comprising N as a heteroatom.

In some embodiments, L¹ is

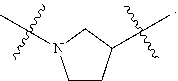 [SMILES: *C1CCN(*)C1]

In some embodiments, L¹ is

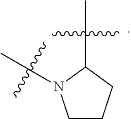 [SMILES: *C1CCCN1*]

In some embodiments, L¹ is 6-membered heterocyclediyl.

In some embodiments, L¹ is 6-membered heterocyclediyl comprising N as a heteroatom.

In some embodiments, L¹ is piperidinediyl.

In some embodiments, L¹ is

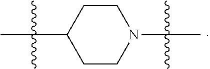 [SMILES: *C1CCN(*)CC1]

In some embodiments, L¹ is

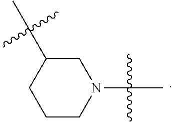 [SMILES: *C1CCCN(*)C1]

In some embodiments, L¹ is

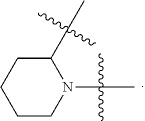 [SMILES: *C1CCCCN1*]

In some embodiments, L¹ is

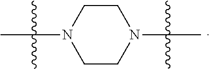 [SMILES: *N1CCN(*)CC1]

In some embodiments, L¹ is

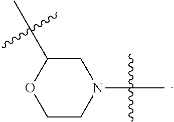 [SMILES: *C1CN(*)CCO1]

In some embodiments, L¹ is

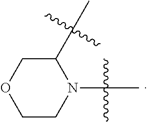 [SMILES: *C1COCCN1*]

In some embodiments, L¹ is 7-membered heterocyclediyl.

In some embodiments, L¹ is 7-membered heterocyclediyl comprising N as a heteroatom.

In some embodiments, L¹ is heterocyclediyl-C₁-C₁₀-alkanediyl.

In some embodiments, L¹ is heterocyclediyl-methanediyl.

In some embodiments, L¹ is heterocyclediyl-ethanediyl.

In some embodiments, L¹ is heterocyclediyl-1,2-ethanediyl.

In some embodiments, L¹ is heterocyclediyl-propanediyl.

In some embodiments, L¹ is heterocyclediyl-1,3-propanediyl.

In some embodiments, L¹ is heterocyclediyl-butanediyl.

In some embodiments, L¹ is heterocyclediyl-1,4-butanediyl.

In some embodiments, L¹ is heterocyclediyl-pentanediyl.

In some embodiments, L¹ is heterocyclediyl-hexanediyl.

In some embodiments, L¹ is heterocyclediyl-heptanediyl.

In some embodiments, L¹ is heterocyclediyl-octanediyl.

In some embodiments, L¹ is heterocyclediyl-nonanediyl.

In some embodiments, L¹ is heterocyclediyl-decanediyl.

In some embodiments, L¹ is 3-membered heterocyclediyl-C₁-C₁₀-alkanediyl.

In some embodiments, L¹ is 4-membered heterocyclediyl-C₁-C₁₀-alkanediyl.

In some embodiments, L¹ is

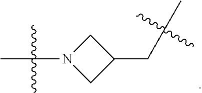 [SMILES: *CC1CN(*)C1]

In some embodiments, L¹ is

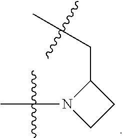 [SMILES: *CC1CCN1*]

In some embodiments, L¹ is

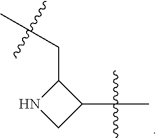 [SMILES: *CC1NCC1*]

In some embodiments, L¹ is

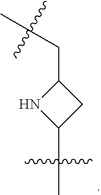 [SMILES: *CC1CC(*)N1]

In some embodiments, L¹ is

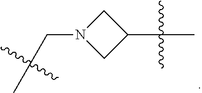 [SMILES: *CN1CC(*)C1]

In some embodiments, L¹ is

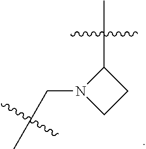 [SMILES: *CN1CCC1*]

In some embodiments, L¹ is

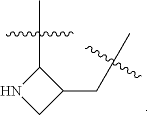 [SMILES: *CC1CNC1*]

In some embodiments, L¹ is

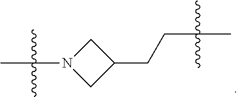 [SMILES: *CCC1CN(*)C1]

In some embodiments, L¹ is 5-membered heterocyclediyl-C₁-C₁₀-alkanediyl.

In some embodiments, L is

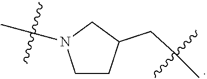 [SMILES: *CC1CCN(*)C1]

In some embodiments, L¹ is

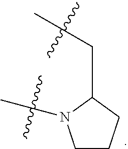 [SMILES: *CC1CCCN1*]

In some embodiments, L¹ is 6-membered heterocyclediyl-C₁-C₁₀-alkanediyl.

In some embodiments, L¹ is

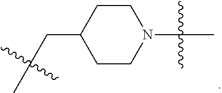 [SMILES: *CC1CCN(*)CC1]

In some embodiments, L¹ is

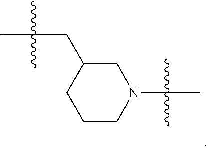 [SMILES: *CC1CCCN(*)C1]

In some embodiments, L¹ is

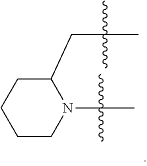 [SMILES: *CC1CCCCN1*]

In some embodiments, L¹ is

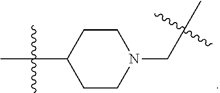 [SMILES: *CN1CCC(*)CC1]

In some embodiments, L¹ is

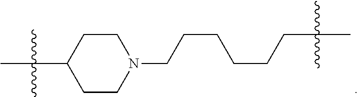 [SMILES: *CCCCCCN1CCC(*)CC1]

In some embodiments, L¹ is

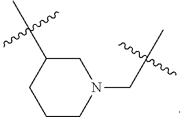 [SMILES: *CN1CCCC(*)C1]

In some embodiments, L¹ is

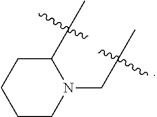 [SMILES: *CN1CCCCC1*]

In some embodiments, L¹ is

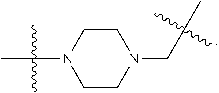 [SMILES: *CN1CCN(*)CC1]

In some embodiments, L¹ is

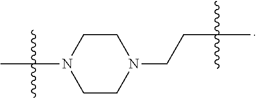 [SMILES: *CCN1CCN(*)CC1]

In some embodiments, L¹ is

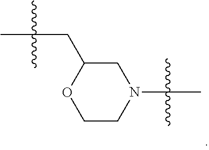 [SMILES: *CC1CN(*)CCO1]

In some embodiments, L¹ is

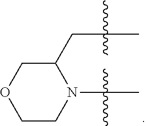 [SMILES: *CC1COCCN1*]

In some embodiments, L¹ is

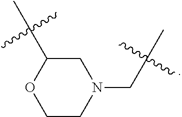 [SMILES: *CN1CCOC(*)C1]

In some embodiments, L¹ is

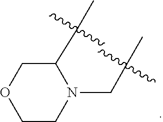 [SMILES: *CN1CCOCC1*]

In some embodiments, L¹ is 7-membered heterocyclediyl-C₁-C₁₀-alkanediyl.

In some embodiments, L¹ is heteroarenediyl.

In some embodiments, L¹ is 5-membered heteroarenediyl.

In some embodiments, L¹ is

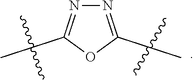 [SMILES: *c1nnc(*)o1]

In some embodiments, L¹ is

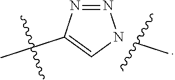

In some embodiments, L¹ is

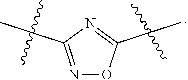 [SMILES: *c1noc(*)n1]

In some embodiments, L¹ is

In some embodiments, L¹ is 6-membered heteroarenediyl.

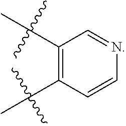 [SMILES: *c1ccncc1*]

In some embodiments, L¹ is

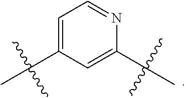 [SMILES: *c1ccnc(*)c1]

In some embodiments, L¹ is

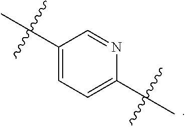 [SMILES: *c1ccc(*)nc1]

In some embodiments, L¹ is

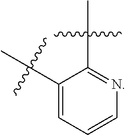 [SMILES: *c1cccnc1*]

In some embodiments, L¹ is heteroarenediyl-C₁-C₁₀ alkanediyl.

In some embodiments, L¹ is heteroarenediyl-methanediyl.

In some embodiments, L¹ is heteroarenediyl-ethanediyl.

In some embodiments, L¹ is heteroarenediyl-1,2-ethanediyl.

In some embodiments, L¹ is heteroarenediyl-propanediyl.

In some embodiments, L¹ is heteroarenediyl-1,3-propanediyl.

In some embodiments, L¹ is heteroarenediyl-butanediyl.

In some embodiments, L¹ is heteroarenediyl-1,4-butanediyl.

In some embodiments, L¹ is heteroarenediyl-pentanediyl.

In some embodiments, L¹ is heteroarenediyl-hexanediyl.

In some embodiments, L¹ is heteroarenediyl-heptanediyl.

In some embodiments, L¹ is heteroarenediyl-octanediyl.

In some embodiments, L¹ is heteroarenediyl-nonanediyl.

In some embodiments, L¹ is heteroarenediyl-decanediyl.

In some embodiments, L¹ is 5-membered heteroarenediyl-C₁-C₁₀ alkanediyl.

In some embodiments, L¹ is

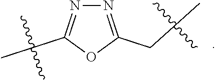 [SMILES: *Cc1nnc(*)o1]

In some embodiments, L¹ is

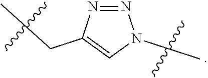 [SMILES: *Cc1cn(*)nn1]

In some embodiments, L¹ is

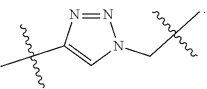 [SMILES: *Cn1cc(*)nn1]

In some embodiments, L¹ is

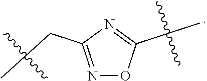 [SMILES: *Cc1noc(*)n1]

In some embodiments, L¹ is

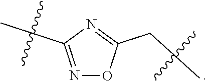 [SMILES: *Cc1nc(*)no1]

In some embodiments, L¹ is 6-membered heteroarenediyl-C₁-C₁₀ alkanediyl.

In some embodiments, L¹ is

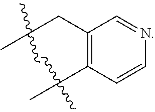 [SMILES: *Cc1cnccc1*]

In some embodiments, L¹ is

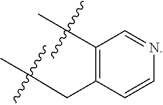 [SMILES: *Cc1ccncc1*]

In some embodiments, L¹ is

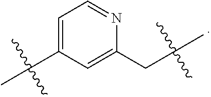 [SMILES: *Cc1cc(*)ccn1]

In some embodiments, L¹ is

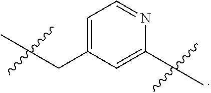 [SMILES: *Cc1ccnc(*)c1]

In some embodiments, L¹ is

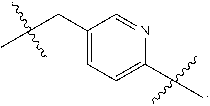 [SMILES: *Cc1ccc(*)nc1]

In some embodiments, L¹ is

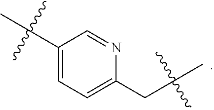 [SMILES: *Cc1ccc(*)cn1]

In some embodiments, L¹ is

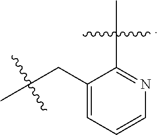 [SMILES: *Cc1cccnc1*]

In some embodiments, L¹ is

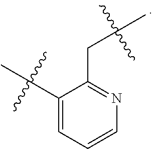 [SMILES: *Cc1ncccc1*]

In some embodiments, L¹ is C₁-C₆ alkanediyl-O—C₁-C₆ alkanediyl.

In some embodiments, L¹ is —CH₂—O—CH₂—.

In some embodiments, L¹ is —CH₂—O—CH₂CH₂—.

In some embodiments, L¹ is —CH₂—O—CH₂CH₂CH₂—.

In some embodiments, L¹ is —CH₂CH₂—O—CH₂CH₂—.

In some embodiments, L¹ is —CH₂CH₂—O—CH₂CH₂CH₂—.

In some embodiments, L¹ is —CH₂CH₂CH₂—O—CH₂CH₂CH₂—.

In some embodiments, L¹ is C₁-C₆ alkanediyl-NR¹—C₁-C₆ alkanediyl.

In some embodiments, L¹ is C₁-C₆ alkanediyl-NH—C₁-C₆ alkanediyl.

In some embodiments, L¹ is C₁-C₆ alkanediyl-N(CH₃)—C₁-C₆ alkanediyl.

In some embodiments, L¹

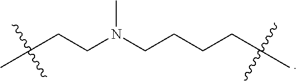 [SMILES: *CCCCN(C)CC*]

In some embodiments, L¹ is,

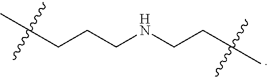 [SMILES: *CCCNCC*]

In some embodiments, L¹ is

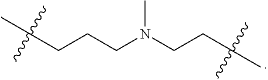 [SMILES: *CCCN(C)CC*]

In some embodiments, L¹ is C₁-C₆ alkanediyl-C(O)NR¹—C₁-C₆ alkanediyl, wherein each alkanediyl is independently optionally substituted with one or more substituents independently selected from halogen, OH, oxo, CN, C₁-C₆ alkyl, C₁-C₆ alkoxy, C₁-C₆ alkyl-C₁-C₆ alkoxy, C₁-C₆ alkyl-NR¹—C₁-C₆ alkyl, NR³R⁴, —C(O)NR³R⁴, —S(O)C₁-C₆ alkyl, —S(O)₂NR³R⁴, aryl, and —S(O)₂C₁-C₆ alkyl.

In some embodiments, L¹ is —CH₂CH₂CH₂NHC(O)CH₂—.

In some embodiments, L¹ is

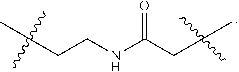 [SMILES: *CCNC(=O)C*]

In some embodiments, L¹ is

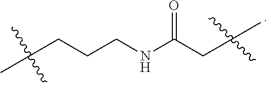 [SMILES: *CCCNC(=O)C*]

In some embodiments, L¹ is

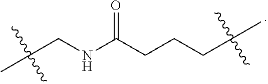 [SMILES: *CCCC(=O)NC*]

In some embodiments, L¹ is —CH₂CH₂CH₂NHC(O)C(CH₃)H—.

In some embodiments, L¹ is

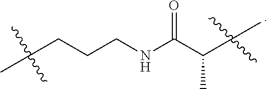 [SMILES: *CCCNC(=O)[C@H](*)C]

In some embodiments, L¹ is

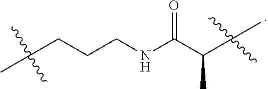 [SMILES: *CCCNC(=O)[C@@H](*)C]

In some embodiments, L¹ is substituted with one or more substituents independently selected from halogen, OH, oxo, CN, C₁-C₆ alkyl, C₁-C₆ alkoxy, C₁-C₆ alkyl-C₁-C₆ alkoxy, C₁-C₆ alkyl-NR¹—C₁-C₆ alkyl, NR³R⁴, —C(O)NR³R⁴, —S(O)C₁-C₆ alkyl, —S(O)₂NR³R⁴, aryl, and —S(O)₂C₁-C₆ alkyl.

In some embodiments, L² is selected from C₁-C₁₀ alkanediyl, C₂-C₁₀ alkenediyl, C₂-C₁₀ alkynediyl, C₃-C₁₀-cycloalkanediyl, C₃-C₁₀-cycloalkanediyl-C₁-C₁₀-alkanediyl, arenediyl, arenediyl-C₁-C₁₀-alkanediyl, heterocyclediyl, heterocyclediyl-C₁-C₁₀-alkanediyl, heterocyclediyl-C(O), heteroarenediyl, heteroarenediyl-C₁-C₁₀ alkanediyl, C₁-C₆ alkanediyl-O—C₁-C₆ alkanediyl, C₁-C₆ alkanediyl-NR¹—C₁-C₆ alkanediyl, C₁-C₆ alkanediyl-C(O)NR¹—C₁-C₆ alkanediyl, wherein alkanediyl, alkendiyl, alkynediyl, arenediyl, heterocyclediyl or heteroarenediyl heteroaryl is optionally substituted with one or more substituents independently selected from halogen, OH, oxo, CN, C₁-C₆ alkyl, C₁-C₆ alkoxy, C₁-C₆ alkyl-C₁-C₆ alkoxy, C₁-C₆ alkyl-NR¹—C₁-C₆ alkyl, NR³R⁴, —C(O)NR³R⁴, —S(O)C₁-C₆ alkyl, —S(O)₂NR³R⁴, aryl, and —S(O)₂C₁-C₆ alkyl.

In some embodiments, L² is C₁-C₁₀ alkanediyl.

In some embodiments, L² is methanediyl.

In some embodiments, L² is ethanediyl.

In some embodiments, L² is 1,1-ethanediyl.

In some embodiments, L² is 1,2-ethanediyl.

In some embodiments, L² is propanediyl.

In some embodiments, L² is 1,3-propanediyl.

In some embodiments, L² is 1-methylpropanediyl-1,3.

In some embodiments, L² is 2-methylpropanediyl-1,3.

In some embodiments, L² is butanediyl.

In some embodiments, L² is 1,3-butanediyl.

In some embodiments, L² is 1,4-butanediyl.

In some embodiments, L² is 2-methylbutanediyl-1,4.

In some embodiments, L² is pentanediyl.

In some embodiments, L² is 1,5-pentanediyl.

In some embodiments, L² is 3-methylpentanediyl-1,5.

In some embodiments, L² is 2,3-dimethylpentanediyl-1,5.

In some embodiments, L² is hexanediyl.

In some embodiments, L² is 1,6-hexanediyl.

In some embodiments, L² is 3-methylhexanediyl-1,6.

In some embodiments, L² is 3,4-dimethylhexanediyl-1,6.

In some embodiments, L² is C₂-C₁₀ alkenediyl.

In some embodiments, L² is ethenediyl.

In some embodiments, L² is ethenediyl-1,2.

In some embodiments, L² is propenediyl.

In some embodiments, L² is propenediyl-1,3.

In some embodiments, L² is 2-methylpropenediyl-1,3

In some embodiments, L² is butenediyl.

In some embodiments, L² is butene-1-diyl-1,4.

In some embodiments, L² is E-butene-1-diyl-1,4.

In some embodiments, L² is Z-butene-1-diyl-1,4.

In some embodiments, L² is butene-2-diyl-1,4.

In some embodiments, L² is E-butene-2-diyl-1,4.

In some embodiments, L² is Z-butene-2-diyl-1,4.

In some embodiments, L² is 2-methylbutene-2-diyl-1,4.

In some embodiments, L² is E-2-methylbutene-2-diyl-1,4.

In some embodiments, L² is Z-2-methylbutene-2-diyl-1,4.

In some embodiments, L² is pentenediyl.

In some embodiments, L² is pentene-2-diyl-1,5.

In some embodiments, L² is E-pentene-2-diyl-1,5.

In some embodiments, L² is Z-pentene-2-diyl-1,5.

In some embodiments, L² is 2-methylpentene-2-diyl-1,5.

In some embodiments, L² is E-2-methylpentene-2-diyl-1,5.

In some embodiments, L² is Z-2-methylpentene-2-diyl-1,5.

In some embodiments, L² is 3-methylpentene-2-diyl-1,5.

In some embodiments, L² is E-3-methylpentene-2-diyl-1,5.

In some embodiments, L² is Z-3-methylpentene-2-diyl-1,5.

In some embodiments, L² is 2,3-dimethylpenten-2-diyl-1,5.

In some embodiments, L² is E-2,3-dimethylpenten-2-diyl-1,5.

In some embodiments, L² is Z-2,3-dimethylpenten-2-diyl-1,5.

In some embodiments, L² is hexenediyl.

In some embodiments, L² is hexene-2-diyl-1,6.

In some embodiments, L² is hexene-3-diyl-1,6.

In some embodiments, L² is 3-methylhexen-3-diyl-1,6.

In some embodiments, L² is E-3-methylhexen-3-diyl-1,6.

In some embodiments, L² is Z-3-methylhexen-3-diyl-1,6.

In some embodiments, L² is 3,4-dimethylhexen-3-diyl-1,6.

In some embodiments, L² is E-3,4-dimethylhexen-3-diyl-1,6.

In some embodiments, L² is Z-3,4-dimethylhexen-3-diyl-1,6.

In some embodiments, L² is C₂-C₁₀ alkynediyl.

In some embodiments, L² is ethynediyl.

In some embodiments, L² is propynediyl.

In some embodiments, L² is propyne-1-diyl-1,3.

In some embodiments, L² is 3-methylpropyne-1-diyl-1,3.

In some embodiments, L² is C₃-C₁₀-cycloalkanediyl.

In some embodiments, L² is

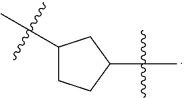 [SMILES: *C1CCC(*)C1]

In some embodiments, L² is

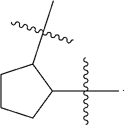 [SMILES: *C1CCCC1*]

In some embodiments, L² is

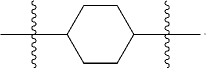 [SMILES: *C1CCC(*)CC1]

In some embodiments, L² is

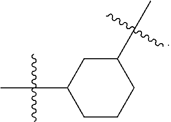 [SMILES: *C1CCCC(*)C1]

In some embodiments, L² is

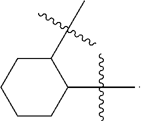 [SMILES: *C1CCCCC1*]

In some embodiments, L² is C₃-C₁₀-cycloalkanediyl-C₁-C₁₀-alkanediyl.

In some embodiments, L² is

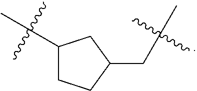 [SMILES: *CC1CCC(*)C1]

In some embodiments, L² is

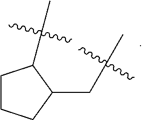 [SMILES: *CC1CCCC1*]

In some embodiments, L² is

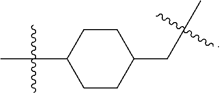 [SMILES: *CC1CCC(*)CC1]

In some embodiments, L² is

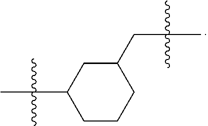 [SMILES: *CC1CCCC(*)C1]

In some embodiments, L² is arenediyl.

In some embodiments, L² is.

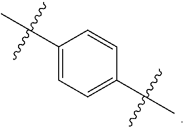 [SMILES: *c1ccc(*)cc1]

In some embodiments, L² is

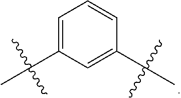 [SMILES: *c1cccc(*)c1]

In some embodiments, L² is

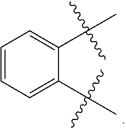 [SMILES: *c1ccccc1*]

In some embodiments, L² is arenediyl-C₁-C₁₀-alkanediyl.

In some embodiments, L² is arenediyl-methanediyl.

In some embodiments, L² is arenediyl-ethanediyl.

In some embodiments, L² is arenediyl-1,2-ethanediyl.

In some embodiments, L² is arenediyl-propanediyl.

In some embodiments, L² is arenediyl-1,3-propanediyl.

In some embodiments, L² is arenediyl-butanediyl.

In some embodiments, L² is arenediyl-1,4-butanediyl.

In some embodiments, L² is arenediyl-pentanediyl.

In some embodiments, L² is arenediyl-hexanediyl.

In some embodiments, L² is arenediyl-heptanediyl.

In some embodiments, L² is arenediyl-octanediyl.

In some embodiments, L² is arenediyl-nonanediyl.

In some embodiments, L² is arenediyl-decanediyl.

In some embodiments, L² is phenylen-C₁-C₁₀-alkanediyl.

In some embodiments, L² is

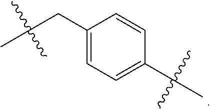 [SMILES: *Cc1ccc(*)cc1]

In some embodiments, L² is

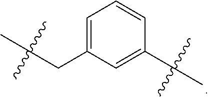 [SMILES: *Cc1cccc(*)c1]

In some embodiments, L² is

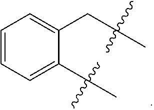 [SMILES: *Cc1ccccc1*]

In some embodiments, L² is heterocyclediyl.

In some embodiments, L² is 3-membered heterocyclediyl.

In some embodiments, L² is 3-membered heterocyclediyl comprising N as a heteroatom.

In some embodiments, L² is 4-membered heterocyclediyl.

In some embodiments, L² is 4-membered heterocyclediyl comprising N as a heteroatom.

In some embodiments, L² is 5-membered heterocyclediyl.

In some embodiments, L² is 5-membered heterocyclediyl comprising N as a heteroatom.

In some embodiments, L² is 6-membered heterocyclediyl.

In some embodiments, L² is 6-membered heterocyclediyl comprising N as a heteroatom.

In some embodiments, L² is piperidinediyl.

In some embodiments, L² is

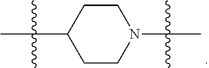 [SMILES: *C1CCN(*)CC1]

In some embodiments, L² is

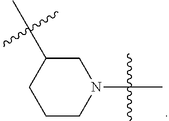 [SMILES: *C1CCCN(*)C1]

In some embodiments, L² is

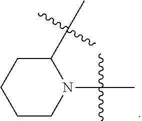 [SMILES: *C1CCCCN1*]

In some embodiments, L² is

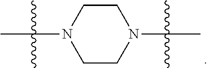 [SMILES: *N1CCN(*)CC1]

In some embodiments, L² is 7-membered heterocyclediyl.

In some embodiments, L² is 7-membered heterocyclediyl comprising N as a heteroatom.

In some embodiments, L² is heterocyclediyl-C₁-C₁₀-alkanediyl.

In some embodiments, L² is heterocyclediyl-methanediyl.

In some embodiments, L² is heterocyclediyl-ethanediyl.

In some embodiments, L² is heterocyclediyl-1,2-ethanediyl.

In some embodiments, L² is heterocyclediyl-propanediyl.

In some embodiments, L² is heterocyclediyl-1,3-propanediyl.

In some embodiments, L² is heterocyclediyl-butanediyl.

In some embodiments, L² is heterocyclediyl-1,4-butanediyl.

In some embodiments, L² is heterocyclediyl-pentanediyl.

In some embodiments, L² is heterocyclediyl-hexanediyl.

In some embodiments, L² is heterocyclediyl-heptanediyl.

In some embodiments, L² is heterocyclediyl-octanediyl.

In some embodiments, L² is heterocyclediyl-nonanediyl.

In some embodiments, L² is heterocyclediyl-decanediyl.

In some embodiments, L² is 3-membered heterocyclediyl-C₁-C₁₀-alkanediyl.

In some embodiments, L² is 4-membered heterocyclediyl-C₁-C₁₀-alkanediyl.

In some embodiments, L² is 5-membered heterocyclediyl-C₁-C₁₀-alkanediyl.

In some embodiments, L² is 6-membered heterocyclediyl-C₁-C₁₀-alkanediyl.

In some embodiments, L² is

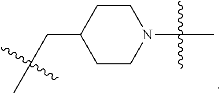 [SMILES: *CC1CCN(*)CC1]

In some embodiments, L² is

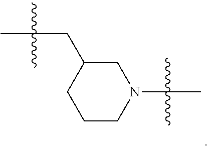 [SMILES: *CC1CCCN(*)C1]

In some embodiments, L² is

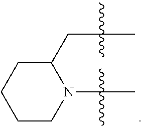 [SMILES: *CC1CCCCN1*]

In some embodiments, L² is

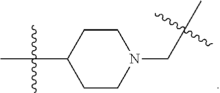 [SMILES: *CN1CCC(*)CC1]

In some embodiments, L² is

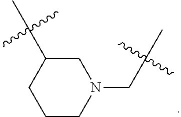 [SMILES: *CN1CCCC(*)C1]

In some embodiments, L² is

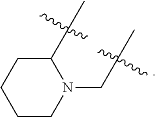 [SMILES: *CN1CCCCC1*]

In some embodiments, L² is 7-membered heterocyclediyl-C₁-C₁₀-alkanediyl.

In some embodiments, L² is heterocyclediyl-C(O).

In some embodiments, L² is

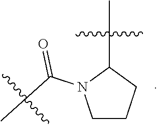 [SMILES: *C(=O)N1CCCC1*]

In some embodiments, L² is

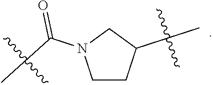 [SMILES: *C(=O)N1CCC(*)C1]

In some embodiments, L²

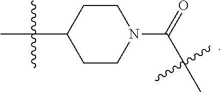 [SMILES: *C(=O)N1CCC(*)CC1]

In some embodiments, L² is

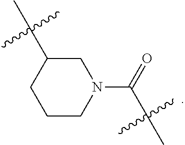 [SMILES: *C(=O)N1CCCC(*)C1]

In some embodiments, L² is

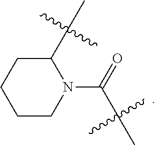 [SMILES: *C(=O)N1CCCCC1*]

In some embodiments, L² is

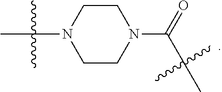 [SMILES: *C(=O)N1CCN(*)CC1]

In some embodiments, L² is heteroarenediyl.

In some embodiments, L² is 5-membered heteroarenediyl.

In some embodiments, L² is

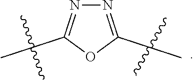 [SMILES: *c1nnc(*)o1]

In some embodiments, L² is

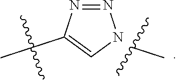

In some embodiments, L² is

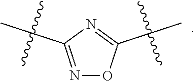 [SMILES: *c1noc(*)n1]

In some embodiments, L² is 6-membered heteroarenediyl.

In some embodiments, L² is

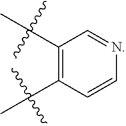 [SMILES: *c1ccncc1*]

In some embodiments, L² is

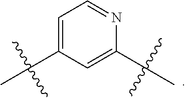 [SMILES: *c1ccnc(*)c1]

In some embodiments, L² is

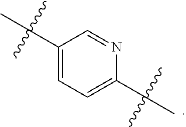 [SMILES: *c1ccc(*)nc1]

In some embodiments, L² is

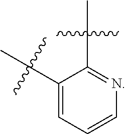 [SMILES: *c1cccnc1*]

In some embodiments, L² is heteroarenediyl-C₁-C₁₀ alkanediyl.

In some embodiments, L² is heteroarenediyl-methanediyl.

In some embodiments, L² is heteroarenediyl-ethanediyl.

In some embodiments, L² is heteroarenediyl-1,2-ethanediyl.

In some embodiments, L² is heteroarenediyl-propanediyl.

In some embodiments, L² is heteroarenediyl-1,3-propanediyl.

In some embodiments, L² is heteroarenediyl-butanediyl.

In some embodiments, L² is heteroarenediyl-1,4-butanediyl.

In some embodiments, L² is heteroarenediyl-pentanediyl.

In some embodiments, L² is heteroarenediyl-hexanediyl.

In some embodiments, L² is heteroarenediyl-heptanediyl.

In some embodiments, L² is heteroarenediyl-octanediyl.

In some embodiments, L² is heteroarenediyl-nonanediyl.

In some embodiments, L² is heteroarenediyl-decanediyl.

In some embodiments, L² is 5-membered heteroarenediyl-C₁-C₁₀ alkanediyl.

In some embodiments, L² is

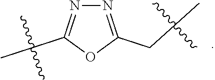 [SMILES: *Cc1nnc(*)o1]

In some embodiments, L² is

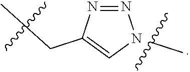

In some embodiments, L² is

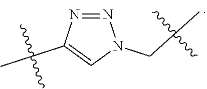 [SMILES: *Cn1cc(*)nn1]

In some embodiments, L²

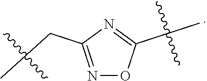 [SMILES: *Cc1noc(*)n1]

In some embodiments, L² is

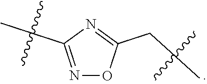 [SMILES: *Cc1nc(*)no1]

In some embodiments, L² is 6-membered heteroarenediyl-C₁-C₁₀ alkanediyl.

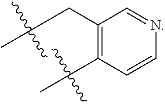 [SMILES: *Cc1cnccc1*]

In some embodiments, L² is

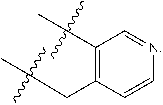 [SMILES: *Cc1ccncc1*]

In some embodiments, L² is

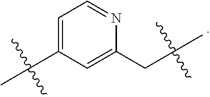 [SMILES: *Cc1cc(*)ccn1]

In some embodiments, L² is

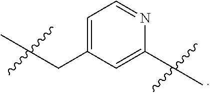 [SMILES: *Cc1ccnc(*)c1]

In some embodiments, L² is

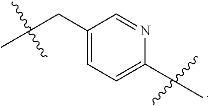 [SMILES: *Cc1ccc(*)nc1]

In some embodiments, L² is

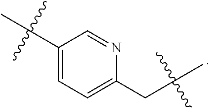 [SMILES: *Cc1ccc(*)cn1]

In some embodiments, L² is

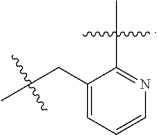 [SMILES: *Cc1cccnc1*]

In some embodiments, L² is

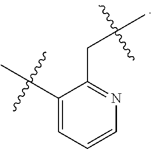 [SMILES: *Cc1ncccc1*]

In some embodiments, L² is C₁-C₆ alkanediyl-O—C₁-C₆ alkanediyl.

In some embodiments, L² is —CH₂—O—CH₂—.

In some embodiments, L² is —CH₂—O—CH₂CH₂—.

In some embodiments, L² is —CH₂—O—CH₂CH₂CH₂—.

In some embodiments, L² is —CH₂CH₂—O—CH₂CH₂—.

In some embodiments, L² is —CH₂CH₂—O—CH₂CH₂CH₂—.

In some embodiments, L² is —CH₂CH₂CH₂—O—CH₂CH₂CH₂—.

In some embodiments, L² is C₁-C₆ alkanediyl-NR¹—C₁-C₆ alkanediyl.

In some embodiments, L² is C₁-C₆ alkanediyl-NH—C₁-C₆ alkanediyl.

In some embodiments, L² is C₁-C₆ alkanediyl-N(CH₃)—C₁-C₆ alkanediyl.

In some embodiments, L² is C₁-C₆ alkanediyl-C(O)NR¹—C₁-C₆ alkanediyl.

In some embodiments, L² is —CH₂—NH—C(O)—CH₂—.

In some embodiments, L² is —CH₂—N(CH₃)—C(O)—CH₂—.

In some embodiments, L² is —CH₂—CH₂—NH—C(O)—CH₂—.

In some embodiments, L² is

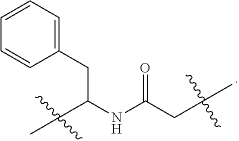 [SMILES: *CC(=O)NC(*)Cc1ccccc1]

In some embodiments, L² is

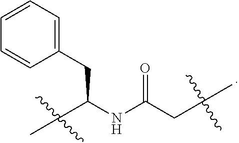 [SMILES: *CC(=O)N[C@@H](*)Cc1ccccc1]

In some embodiments, L² is

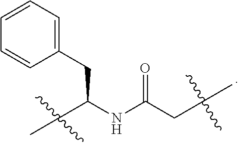 [SMILES: *CC(=O)N[C@@H](*)Cc1ccccc1]

In some embodiments, L² is substituted with one or more substituents independently selected from halogen, OH, oxo, CN, C₁-C₆ alkyl, C₁-C₆ alkoxy, C₁-C₆ alkyl-C₁-C₆ alkoxy, C₁-C₆ alkyl-NH—C₁-C₆ alkyl, NR³R⁴, —C(O)NR³R⁴, —S(O)C₁-C₆ alkyl, —S(O)₂NR³R⁴, and —S(O)₂C₁-C₆ alkyl.

In some embodiments, R³ and R⁴ are each independently selected from H, C₁-C₆ alkyl, C₁-C₆ acyl and C₃-C₁₀ cycloalkyl.

In some embodiments, R³ and R⁴ are H.

In some embodiments, R³ is H and R⁴ is CH₃.

In some embodiments, R³ and R⁴ are CH₃.

In some embodiments, R³ and R⁴ together with the atoms to which they are attached and any intervening atoms, form a 5-14 membered heterocycle, wherein the heterocycle is optionally substituted with one or more substituents independently selected from halogen, OH, ═O, CN, NO₂, CN—C₆ alkyl, CN—C₆ alkoxy.

In some preferred embodiments, combination of X, Y, Z, L¹, and L² is selected from the Table 1.

Table 1. Selection of possible values of X, Y, Z, L¹, and L².

TABLE 1
Selection of possible values of X, Y, Z, L¹, and L².

| X | L¹ | Y | L² | Z |
|---|---|---|---|---|
| bond | CH₂ | bond | CH₂ | bond |
| O | 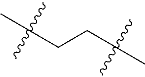 [SMILES: *CC*] | O | 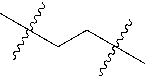 [SMILES: *CC*] | O |
| NH | 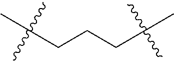 [SMILES: *CCC*] | NH | 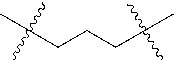 [SMILES: *CCC*] | CH₂ |
| N(CH₃) | 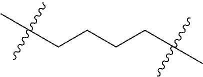 [SMILES: *CCCC*] | N(CH₃) | 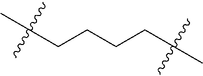 [SMILES: *CCCC*] | |
| CH₂ | 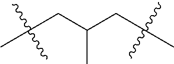 [SMILES: *CC(C)C*] | CH₂ | 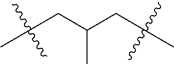 [SMILES: *CC(C)C*] | |
| S | 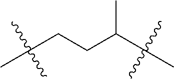 [SMILES: *CCC(*)C] | S | 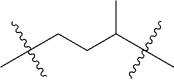 [SMILES: *CCC(*)C] | |
| S(O) | 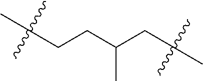 [SMILES: *CCC(C)C*] | S(O) | 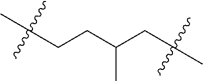 [SMILES: *CCC(C)C*] | |
| S(O)₂ | 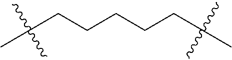 [SMILES: *CCCCC*] | S(O)₂ | 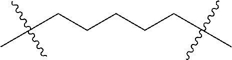 [SMILES: *CCCCC*] | |
| C(O) | 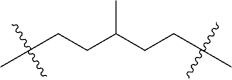 [SMILES: *CCC(C)CC*] | C(O) | 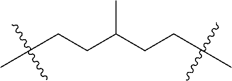 [SMILES: *CCC(C)CC*] | |
| C(O)O | 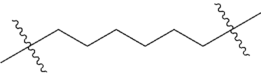 [SMILES: *CCCCCC*] | C(O)O | 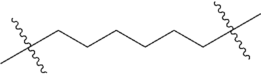 [SMILES: *CCCCCC*] | |
| C(O)NH | 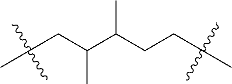 [SMILES: *CCC(C)C(C)C*] | C(O)NH | 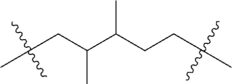 [SMILES: *CCC(C)C(C)C*] | |
| OC(O)O | 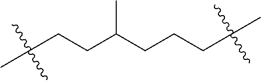 [SMILES: *CCCC(C)CC*] | OC(O)O | 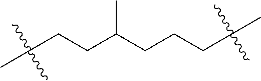 [SMILES: *CCCC(C)CC*] | |
| NHC(O)NH | 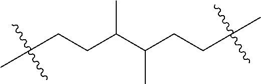 [SMILES: *CCC(C)C(C)CC*] | NHC(O)NH | 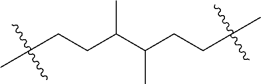 [SMILES: *CCC(C)C(C)CC*] | |
| | 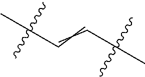 [SMILES: *C=C*] | NCH(CH₃)₂ | 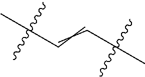 [SMILES: *C=C*] | |
| | 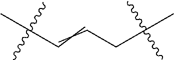 [SMILES: *C=CC*] | | 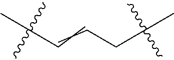 [SMILES: *C=CC*] | |
| | 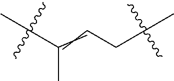 [SMILES: *CC=C(*)C] | | 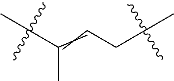 [SMILES: *CC=C(*)C] | |
| | 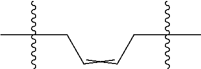 [SMILES: *CC=CC*] | | 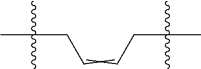 [SMILES: *CC=CC*] | |
| | 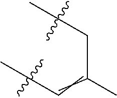 [SMILES: *C=C(C)C*] | | 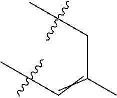 [SMILES: *C=C(C)C*] | |
| | 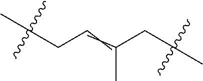 [SMILES: *CC=C(C)C*] | | 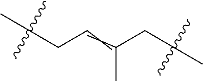 [SMILES: *CC=C(C)C*] | |
| | 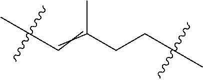 [SMILES: *C=C(C)CC*] | | 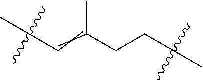 [SMILES: *C=C(C)CC*] | |
| | 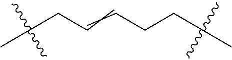 [SMILES: *CC=CCC*] | | 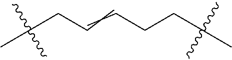 [SMILES: *CC=CCC*] | |
| | 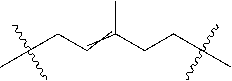 [SMILES: *CC=C(C)CC*] | | 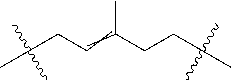 [SMILES: *CC=C(C)CC*] | |
| | 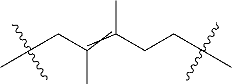 [SMILES: *CCC(C)=C(C)C*] | | 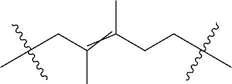 [SMILES: *CCC(C)=C(C)C*] | |
| | 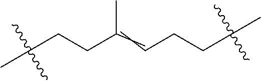 [SMILES: *CCC=C(C)CC*] | | 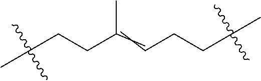 [SMILES: *CCC=C(C)CC*] | |
| | 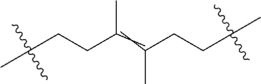 [SMILES: *CCC(C)=C(C)CC*] | | 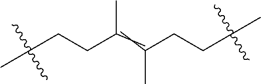 [SMILES: *CCC(C)=C(C)CC*] | |
| | 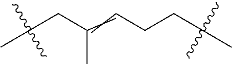 [SMILES: *CCC=C(C)C*] | | 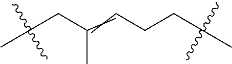 [SMILES: *CCC=C(C)C*] | |
| | 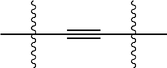 [SMILES: *C#C*] | | 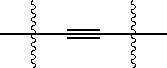 [SMILES: *C#C*] | |
| | 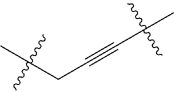 [SMILES: *C#CC*] | | 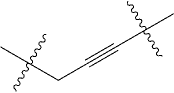 [SMILES: *C#CC*] | |
| | 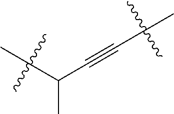 [SMILES: *C#CC(*)C] | | 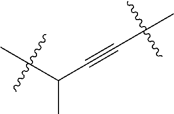 [SMILES: *C#CC(*)C] | |
| | —CH₂—O—CH₂— | | —CH₂—O—CH₂— | |
| | —CH₂—O—CH₂CH₂— | | —CH₂—O—CH₂CH₂— | |
| | —(CH₂)₂—O—(CH₂)₂— | | —(CH₂)₂—O—(CH₂)₂— | |
| | —(CH₂)₃—O—(CH₂)₂— | | —(CH₂)₃—O—(CH₂)₂— | |
| | —(CH₂)₃—O—(CH₂)₄— | | —(CH₂)₃—O—(CH₂)₄— | |
| | —CH₂C(O)NHCH₂— | | —CH₂C(O)NHCH₂— | |
| | —CH₂C(O)N(CH₃)CH₂— | | —CH₂C(O)N(CH₃)CH₂— | |
| | —CH₂C(O)NHCH(CH₃)— | | —CH₂C(O)NHCH(CH₃)— | |
| | —CH₂C(O)NH(CH₂)₂— | | —CH₂C(O)NH(CH₂)₂— | |
| | —CH₂C(O)NHCH(CH₂Ph)— | | —CH₂C(O)NHCH(CH₂Ph)— | |
| | 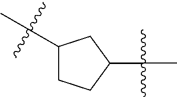 [SMILES: *C1CCC(*)C1] | | 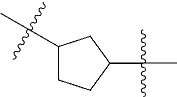 [SMILES: *C1CCC(*)C1] | |
| | 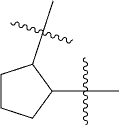 [SMILES: *C1CCCC1*] | | 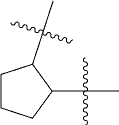 [SMILES: *C1CCCC1*] | |
| | 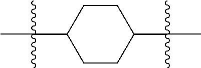 [SMILES: *C1CCC(*)CC1] | | 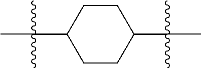 [SMILES: *C1CCC(*)CC1] | |
| | 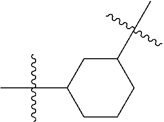 [SMILES: *C1CCCC(*)C1] | | 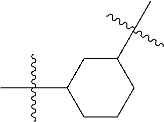 [SMILES: *C1CCCC(*)C1] | |
| | 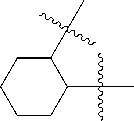 [SMILES: *C1CCCCC1*] | | 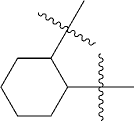 [SMILES: *C1CCCCC1*] | |
| | 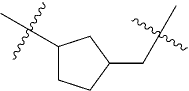 [SMILES: *CC1CCC(*)C1] | | 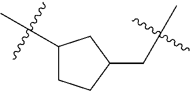 [SMILES: *CC1CCC(*)C1] | |
| | 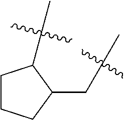 [SMILES: *CC1CCCC1*] | | 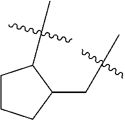 [SMILES: *CC1CCCC1*] | |
| | 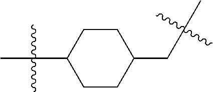 [SMILES: *CC1CCC(*)CC1] | | 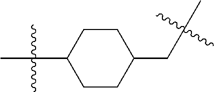 [SMILES: *CC1CCC(*)CC1] | |
| | 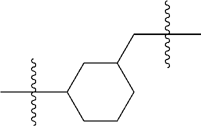 [SMILES: *CC1CCCC(*)C1] | | 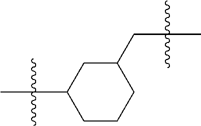 [SMILES: *CC1CCCC(*)C1] | |
| | 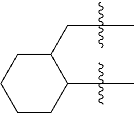 [SMILES: *CC1CCCCC1*] | | 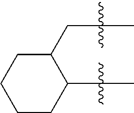 [SMILES: *CC1CCCCC1*] | |
| | 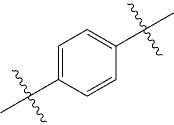 [SMILES: *c1ccc(*)cc1] | | 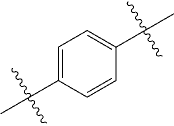 [SMILES: *c1ccc(*)cc1] | |
| | 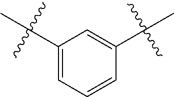 [SMILES: *c1cccc(*)c1] | | 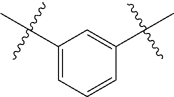 [SMILES: *c1cccc(*)c1] | |
| | 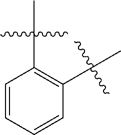 [SMILES: *c1ccccc1*] | | 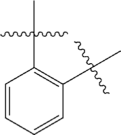 [SMILES: *c1ccccc1*] | |
| | 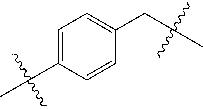 [SMILES: *Cc1ccc(*)cc1] | | 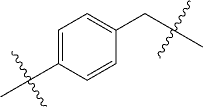 [SMILES: *Cc1ccc(*)cc1] | |
| | 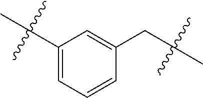 [SMILES: *Cc1cccc(*)c1] | | 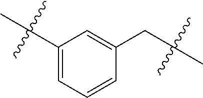 [SMILES: *Cc1cccc(*)c1] | |
| | 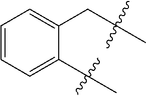 [SMILES: *Cc1ccccc1*] | | 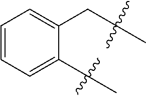 [SMILES: *Cc1ccccc1*] | |
| | 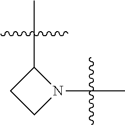 [SMILES: *C1CCN1*] | | 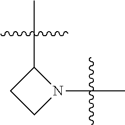 [SMILES: *C1CCN1*] | |
| | 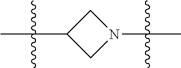 [SMILES: *C1CN(*)C1] | | 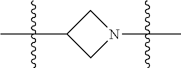 [SMILES: *C1CN(*)C1] | |
| | 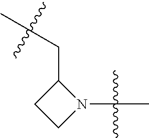 [SMILES: *CC1CCN1*] | | 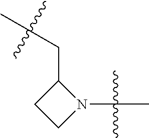 [SMILES: *CC1CCN1*] | |
| | 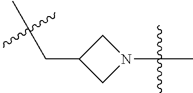 [SMILES: *CC1CN(*)C1] | | 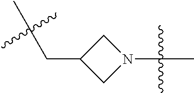 [SMILES: *CC1CN(*)C1] | |
| | 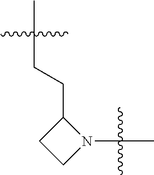 [SMILES: *CCC1CCN1*] | | 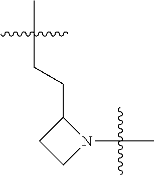 [SMILES: *CCC1CCN1*] | |
| | 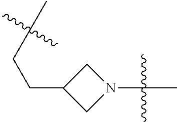 [SMILES: *CCC1CN(*)C1] | | 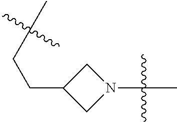 [SMILES: *CCC1CN(*)C1] | |
| | 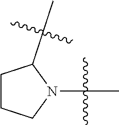 [SMILES: *C1CCCN1*] | | 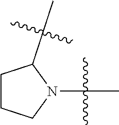 [SMILES: *C1CCCN1*] | |
| | 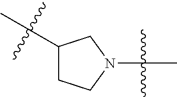 [SMILES: *C1CCN(*)C1] | | 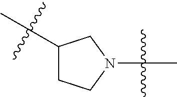 [SMILES: *C1CCN(*)C1] | |
| | 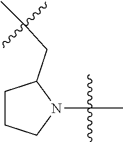 [SMILES: *CC1CCCN1*] | | 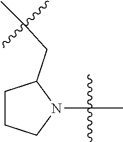 [SMILES: *CC1CCCN1*] | |
| | 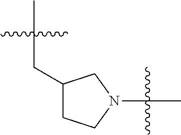 [SMILES: *CC1CCN(*)C1] | | 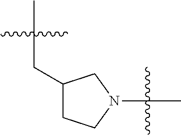 [SMILES: *CC1CCN(*)C1] | |
| | 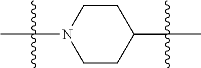 [SMILES: *C1CCN(*)CC1] | | 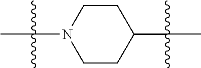 [SMILES: *C1CCN(*)CC1] | |
| | 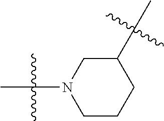 [SMILES: *C1CCCN(*)C1] | | 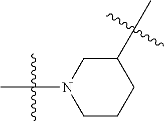 [SMILES: *C1CCCN(*)C1] | |
| | 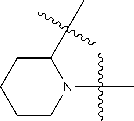 [SMILES: *C1CCCCN1*] | | 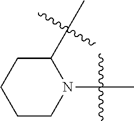 [SMILES: *C1CCCCN1*] | |
| | 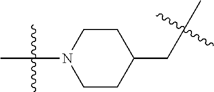 [SMILES: *CC1CCN(*)CC1] | | 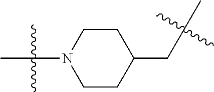 [SMILES: *CC1CCN(*)CC1] | |
| | 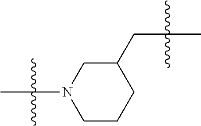 [SMILES: *CC1CCCN(*)C1] | | 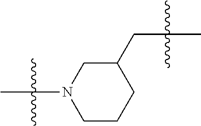 [SMILES: *CC1CCCN(*)C1] | |
| | 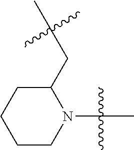 [SMILES: *CC1CCCCN1*] | | 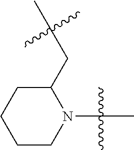 [SMILES: *CC1CCCCN1*] | |
| | 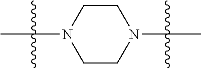 [SMILES: *N1CCN(*)CC1] | | 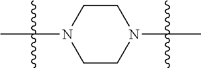 [SMILES: *N1CCN(*)CC1] | |
| | 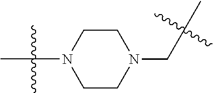 [SMILES: *CN1CCN(*)CC1] | | 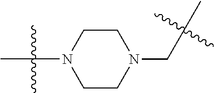 [SMILES: *CN1CCN(*)CC1] | |
| | 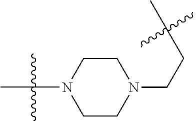 [SMILES: *CCN1CCN(*)CC1] | | 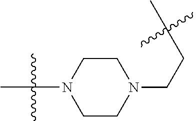 [SMILES: *CCN1CCN(*)CC1] | |
| | 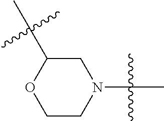 [SMILES: *C1CN(*)CCO1] | | 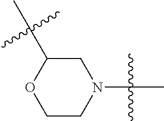 [SMILES: *C1CN(*)CCO1] | |
| | 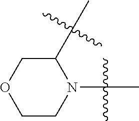 [SMILES: *C1COCCN1*] | | 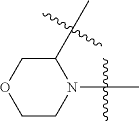 [SMILES: *C1COCCN1*] | |
| | 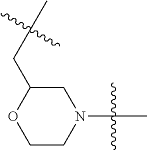 [SMILES: *CC1CN(*)CCO1] | | 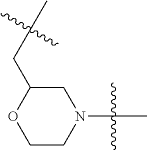 [SMILES: *CC1CN(*)CCO1] | |
| | 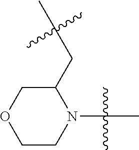 [SMILES: *CC1COCCN1*] | | 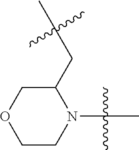 [SMILES: *CC1COCCN1*] | |
| | 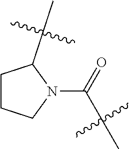 [SMILES: *C(=O)N1CCCC1*] | | 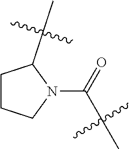 [SMILES: *C(=O)N1CCCC1*] | |
| | 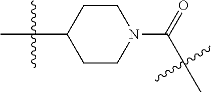 [SMILES: *C(=O)N1CCC(*)CC1] | | 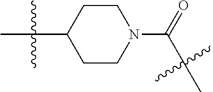 [SMILES: *C(=O)N1CCC(*)CC1] | |
| | 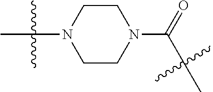 [SMILES: *C(=O)N1CCN(*)CC1] | | 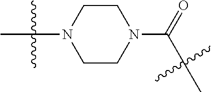 [SMILES: *C(=O)N1CCN(*)CC1] | |
| | 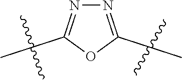 [SMILES: *c1nnc(*)o1] | | 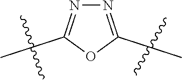 [SMILES: *c1nnc(*)o1] | |
| | 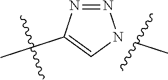 | | 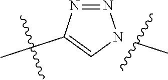 | |
| | 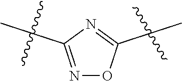 [SMILES: *c1noc(*)n1] | | 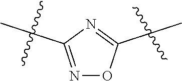 [SMILES: *c1noc(*)n1] | |
| | 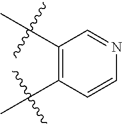 [SMILES: *c1ccncc1*] | | 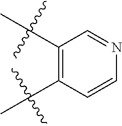 [SMILES: *c1ccncc1*] | |
| | 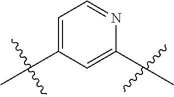 [SMILES: *c1ccnc(*)c1] | | 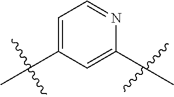 [SMILES: *c1ccnc(*)c1] | |
| | 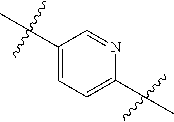 [SMILES: *c1ccc(*)nc1] | | 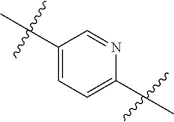 [SMILES: *c1ccc(*)nc1] | |
| | 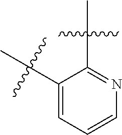 [SMILES: *c1cccnc1*] | | 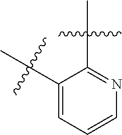 [SMILES: *c1cccnc1*] | |
| | 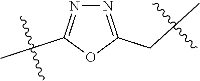 [SMILES: *Cc1nnc(*)o1] | | 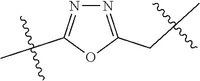 [SMILES: *Cc1nnc(*)o1] | |
| | 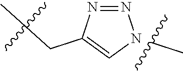 | | 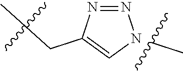 | |
| | 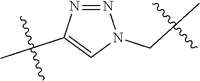 [SMILES: *Cn1cc(*)nn1] | | 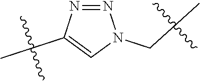 [SMILES: *Cn1cc(*)nn1] | |
| | 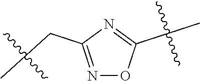 [SMILES: *Cc1noc(*)n1] | | 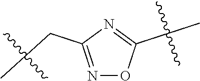 [SMILES: *Cc1noc(*)n1] | |
| | 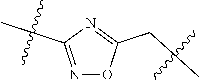 [SMILES: *Cc1nc(*)no1] | | 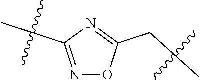 [SMILES: *Cc1nc(*)no1] | |
| | 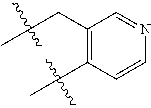 [SMILES: *Cc1cnccc1*] | | 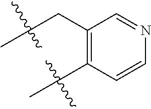 [SMILES: *Cc1cnccc1*] | |
| | 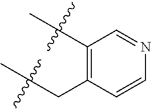 [SMILES: *Cc1ccncc1*] | | 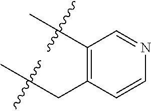 [SMILES: *Cc1ccncc1*] | |
| | 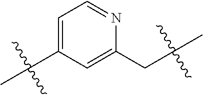 [SMILES: *Cc1cc(*)ccn1] | | 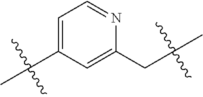 [SMILES: *Cc1cc(*)ccn1] | |
| | 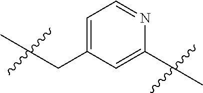 [SMILES: *Cc1ccnc(*)c1] | | 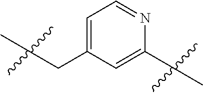 [SMILES: *Cc1ccnc(*)c1] | |
| | 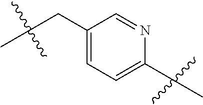 [SMILES: *Cc1ccc(*)nc1] | | 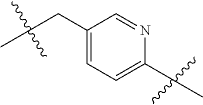 [SMILES: *Cc1ccc(*)nc1] | |
| | 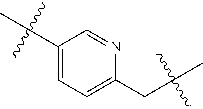 [SMILES: *Cc1ccc(*)cn1] | | 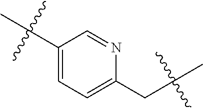 [SMILES: *Cc1ccc(*)cn1] | |
| | 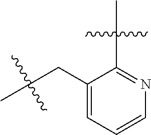 [SMILES: *Cc1cccnc1*] | | 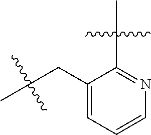 [SMILES: *Cc1cccnc1*] | |
| | 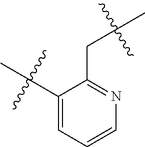 [SMILES: *Cc1ncccc1*] | | 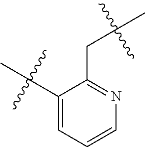 [SMILES: *Cc1ncccc1*] | |

It would be understood by a person of ordinary skill in the art that any of the biradicals described herein can be bonded to any of biradicals described herein according to the General Formula (A) using any of the two unpaired valence electrons, provided such bond is chemically capable of being formed. Some examples of possible bonding biradicals described herein presented in the Table 2.

TABLE 2
Possible bonding of biradicals described herein (non-limiting examples).

| X | L¹ | Y | L² | Z |
|---|---|---|---|---|
| —O— | 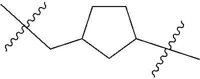 [SMILES: *CC1CCC(*)C1] | —O— | 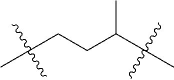 [SMILES: *CCC(*)C] | bond |

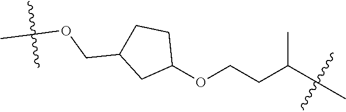 [SMILES: *OCC1CCC(OCCC(*)C)C1]

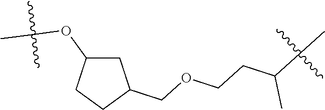 [SMILES: *OC1CCC(COCCC(*)C)C1]

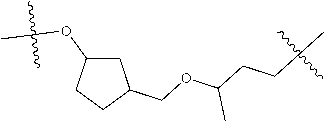 [SMILES: *CCC(C)OCC1CCC(O*)C1]

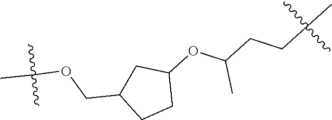 [SMILES: *CCC(C)OC1CCC(CO*)C1]

| | | | | |
|---|---|---|---|---|
| —O— | 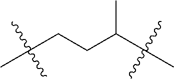 [SMILES: *CCC(*)C] | —C(O)NH— | —CH₂—CH₂— | bond |

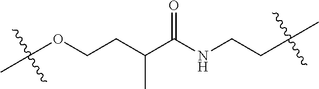 [SMILES: *CCNC(=O)C(C)CCO*]

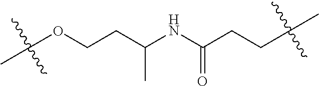 [SMILES: *CCC(=O)NC(C)CCO*]

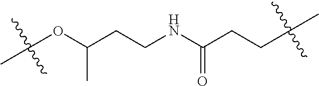 [SMILES: *CCC(=O)NCCC(C)O*]

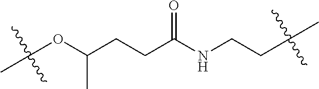 [SMILES: *CCNC(=O)CCC(C)O*]

| | | | | |
|---|---|---|---|---|
| —NH— | 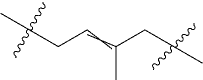 [SMILES: *CC=C(C)C*] | bond | 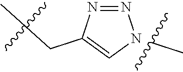 | bond |

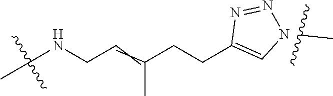

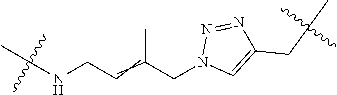 [SMILES: *Cc1cn(CC(C)=CCN*)nn1]

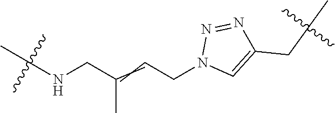 [SMILES: *Cc1cn(CC=C(C)CN*)nn1]

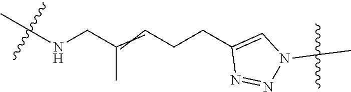 [SMILES: *NCC(C)=CCCc1cn(*)nn1]

In some embodiments, the compound is of Formula (I′):

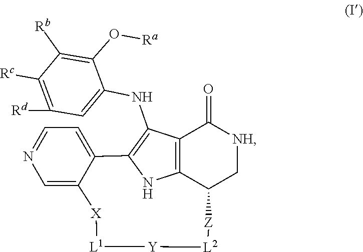

or a pharmaceutically acceptable salt, stereoisomer, solvate, prodrug, isotopic derivative, or tautomer thereof wherein all variables are as defined herein.

In some embodiments, the compound is of Formula (I″):

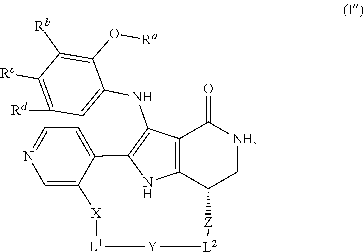

or a pharmaceutically acceptable salt, stereoisomer, solvate, prodrug, isotopic derivative, or tautomer thereof wherein all variables are as defined herein.

In some embodiments, the compound is of Formula (I-A′):

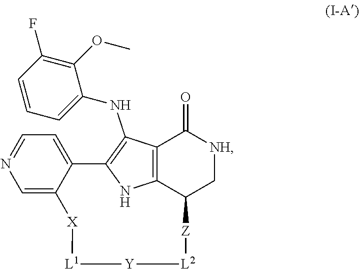

or a pharmaceutically acceptable salt, stereoisomer, solvate, prodrug, isotopic derivative, or tautomer thereof wherein all variables are as defined herein.

In some embodiments, the compound is of Formula (I-A″):

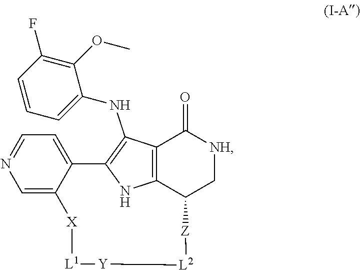

or a pharmaceutically acceptable salt, stereoisomer, solvate, prodrug, isotopic derivative, or tautomer thereof wherein all variables are as defined herein.

In some embodiments, the compound is of Formula (I-A-I):

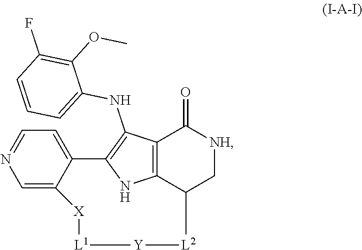

or a pharmaceutically acceptable salt, stereoisomer, solvate, prodrug, isotopic derivative, or tautomer thereof.

In some embodiments, the compound is of Formula (I-A-I′):

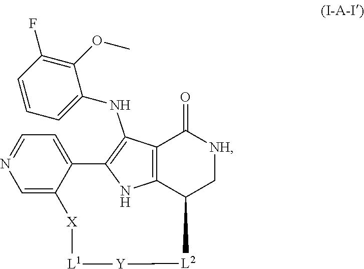

or a pharmaceutically acceptable salt, stereoisomer, solvate, prodrug, isotopic derivative, or tautomer thereof.

In some embodiments, the compound is of Formula (I-A-I″):

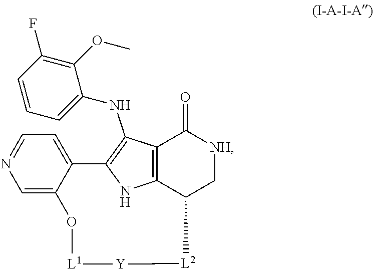

or a pharmaceutically acceptable salt, stereoisomer, solvate, prodrug, isotopic derivative, or tautomer thereof.

In some embodiments, the compound is of Formula (I-A-I-A):

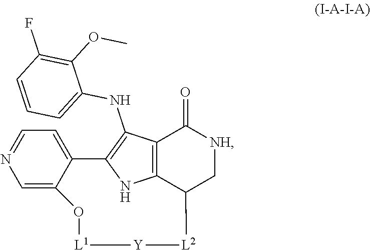

or a pharmaceutically acceptable salt, stereoisomer, solvate, prodrug, isotopic derivative, or tautomer thereof.

In some embodiments, the compound is of Formula (I-A-I-A′):

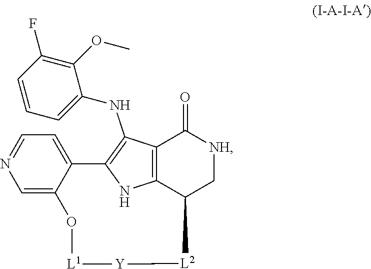

or a pharmaceutically acceptable salt, stereoisomer, solvate, pro rug, isotopic derivative, or tautomer thereof.

In some embodiments, the compound is of Formula (I-A-I-A″):

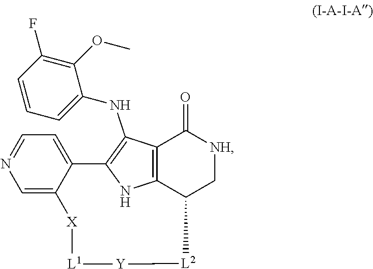

or a pharmaceutically acceptable salt, stereoisomer, solvate, prodrug, isotopic derivative, or tautomer thereof.

In some embodiments, the compound is of Formula (I-A-I-B):

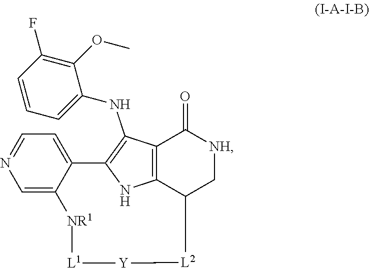

or a pharmaceutically acceptable salt, stereoisomer, solvate, prodrug, isotopic derivative, or tautomer thereof.

In some embodiments, the compound is of Formula (I-A-I-B′):

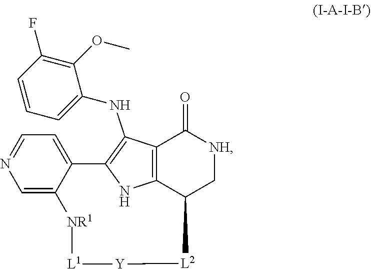

or a pharmaceutically acceptable salt, stereoisomer, solvate, prodrug, isotopic derivative, or tautomer thereof.

In some embodiments, the compound is of Formula (I-A-I-B″):

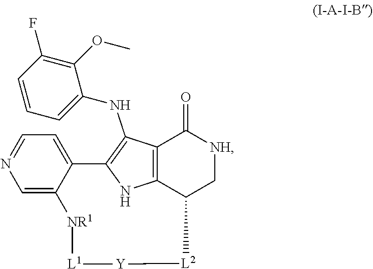

or a pharmaceutically acceptable salt, stereoisomer, solvate, prodrug, isotopic derivative, or tautomer thereof.

In some embodiments, the compound is of Formula (I-A-I-B-H):

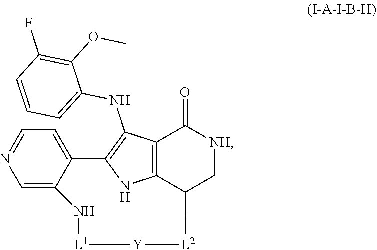

or a pharmaceutically acceptable salt, stereoisomer, solvate, prodrug, isotopic derivative, or tautomer thereof.

In some embodiments, the compound is of Formula (I-A-I-B-H′):

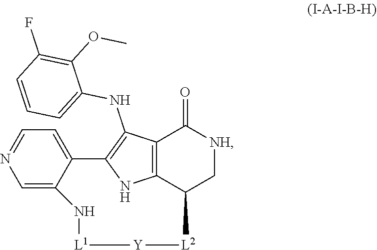

or a pharmaceutically acceptable salt, stereoisomer, solvate, prodrug, isotopic derivative, or tautomer thereof.

In some embodiments, the compound is of Formula (I-A-I-B-H″):

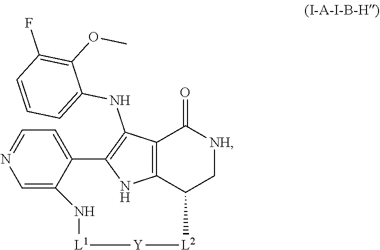

or a pharmaceutically acceptable salt, stereoisomer, solvate, prodrug, isotopic derivative, or tautomer thereof.

In some embodiments, the compound is of Formula (I-A-I-C):

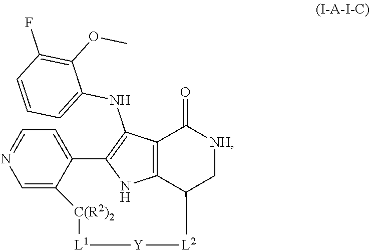

or a pharmaceutically acceptable salt, stereoisomer, solvate, prodrug, isotopic derivative, or tautomer thereof.

In some embodiments, the compound is of Formula (I-A-I-C′):

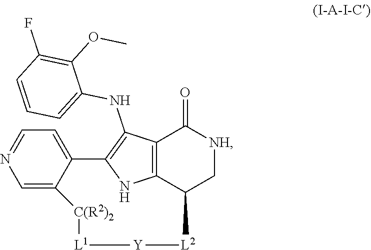

or a pharmaceutically acceptable salt, stereoisomer, solvate, prodrug, isotopic derivative, or tautomer thereof.

In some embodiments, the compound is of Formula (I-A-I-C″):

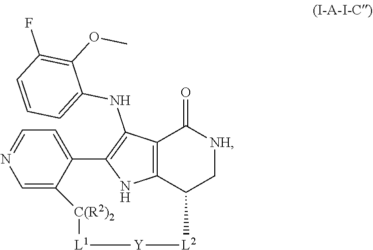

or a pharmaceutically acceptable salt, stereoisomer, solvate, prodrug, isotopic derivative, or tautomer thereof.

In some embodiments, the compound is of Formula (I-A-I-C-H):

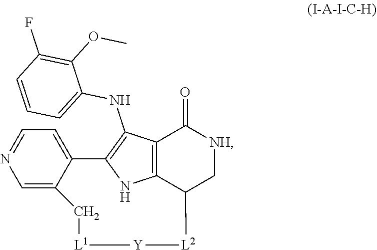

or a pharmaceutically acceptable salt, stereoisomer, solvate, prodrug, isotopic derivative, or tautomer thereof.

In some embodiments, the compound is of Formula (I-A-I-C-H′):

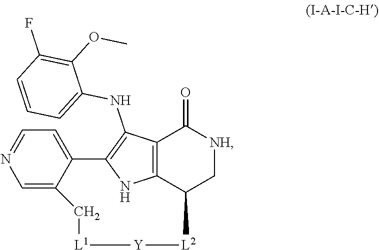

or a pharmaceutically acceptable salt, stereoisomer, solvate, prodrug, isotopic derivative, or tautomer thereof.

In some embodiments, the compound is of Formula (I-A-I-C-H″):

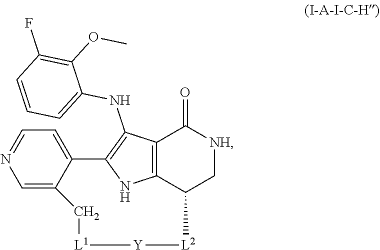

or a pharmaceutically acceptable salt, stereoisomer, solvate, prodrug, isotopic derivative, or tautomer thereof.

In some embodiments, the compound is of Formula (I-A-I-D):

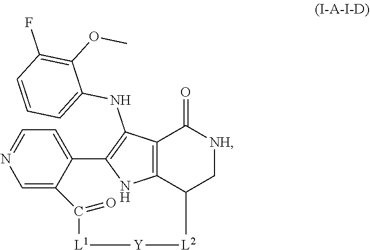

or a pharmaceutically acceptable salt, stereoisomer, solvate, prodrug, isotopic derivative, or tautomer thereof.

In some embodiments, the compound is of Formula (I-A-I-D′):

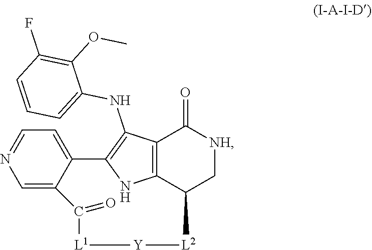

or a pharmaceutically acceptable salt, stereoisomer, solvate, prodrug, isotopic derivative, or tautomer thereof.

In some embodiments, the compound is of Formula (I-A-I-D″):

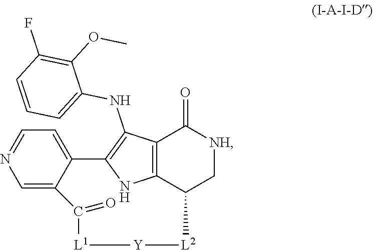

or a pharmaceutically acceptable salt, stereoisomer, solvate, prodrug, isotopic derivative, or tautomer thereof.

In some embodiments, the compound is of Formula (I-A-I-E):

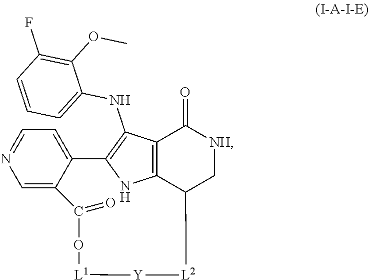

or a pharmaceutically acceptable salt, stereoisomer, solvate, prodrug, isotopic derivative, or tautomer thereof.

In some embodiments, the compound is of Formula (I-A-I-E′):

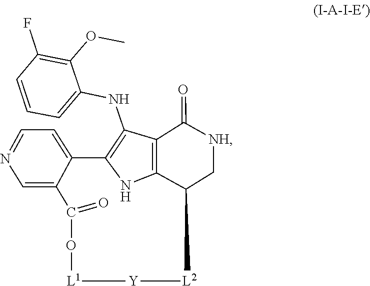

or a pharmaceutically acceptable salt, stereoisomer, solvate, prodrug, isotopic derivative, or tautomer thereof.

In some embodiments, the compound is of Formula (I-A-I-E″):

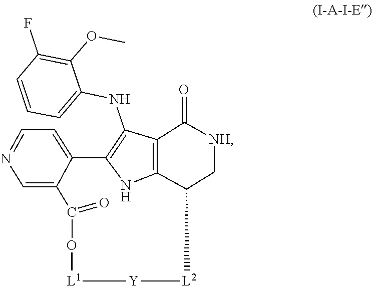

or a pharmaceutically acceptable salt, stereoisomer, solvate, prodrug, isotopic derivative, or tautomer thereof.

In some embodiments, the compound is of Formula (I-A-I-F):

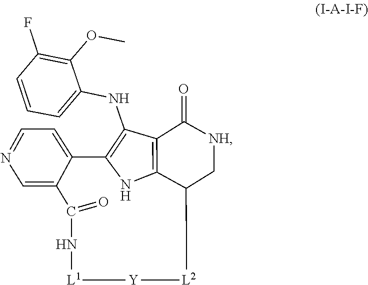

or a pharmaceutically acceptable salt, stereoisomer, solvate, prodrug, isotopic derivative, or tautomer thereof.

In some embodiments, the compound is of Formula (I-A-I-F′):

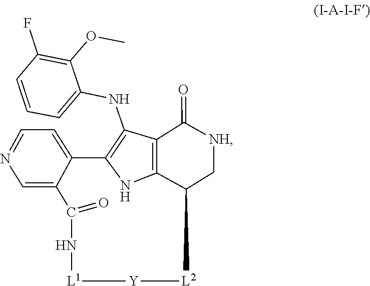

or a pharmaceutically acceptable salt, stereoisomer, solvate, prodrug, isotopic derivative, or tautomer thereof.

In some embodiments, the compound is of Formula (I-A-I-F″):

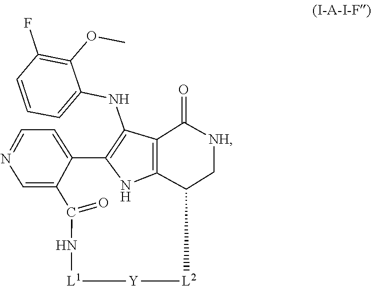

or a pharmaceutically acceptable salt, stereoisomer, solvate, prodrug, isotopic derivative, or tautomer thereof.

In some embodiments, the compound is of Formula (I-A-I-G):

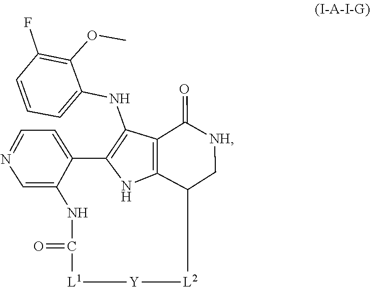

or a pharmaceutically acceptable salt, stereoisomer, solvate, prodrug, isotopic derivative, or tautomer thereof.

In some embodiments, the compound is of Formula (I-A-I-G′):

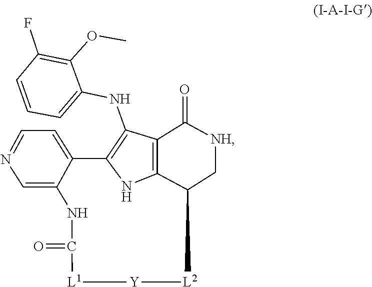

or a pharmaceutically acceptable salt, stereoisomer, solvate, prodrug, isotopic derivative, or tautomer thereof.

In some embodiments, the compound is of Formula (I-A-I-G″):

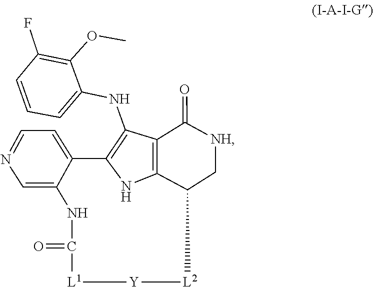

or a pharmaceutically acceptable salt, stereoisomer, solvate, prodrug, isotopic derivative, or tautomer thereof.

In some embodiments, the compound is of Formula (I-A-I-A-a):

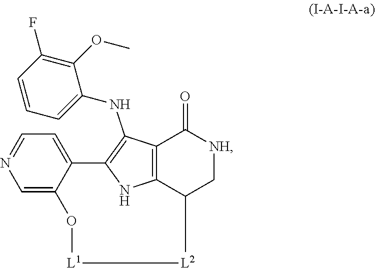

or a pharmaceutically acceptable salt, stereoisomer, solvate, prodrug, isotopic derivative, or tautomer thereof.

In some embodiments, the compound is of Formula (I-A-I-A-a′):

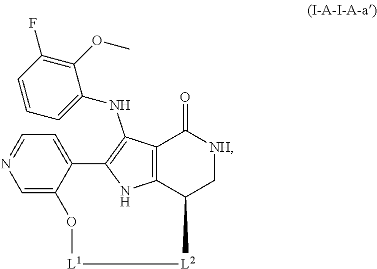

or a pharmaceutically acceptable salt, stereoisomer, solvate, prodrug, isotopic derivative, or tautomer thereof.

In some embodiments, the compound is of Formula (I-A-I-A-a″):

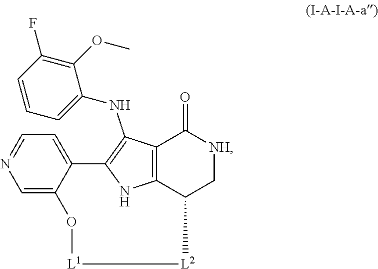

or a pharmaceutically acceptable salt, stereoisomer, solvate, prodrug, isotopic derivative, or tautomer thereof.

In some embodiments, the compound is of Formula (I-A-I-A-b):

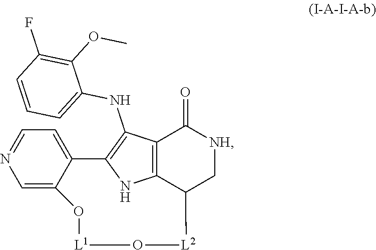

or a pharmaceutically acceptable salt, stereoisomer, solvate, prodrug, isotopic derivative, or tautomer thereof.

In some embodiments, the compound is of Formula (I-A-I-A-b′):

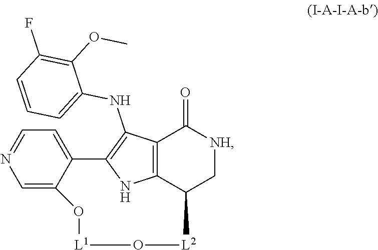

or a pharmaceutically acceptable salt, stereoisomer, solvate, prodrug, isotopic derivative, or tautomer thereof.

In some embodiments, the compound is of Formula (I-A-I-A-b″):

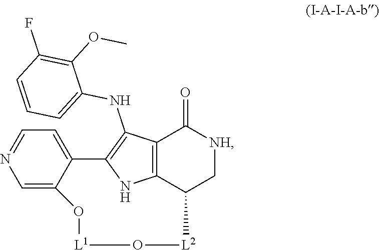

or a pharmaceutically acceptable salt, stereoisomer, solvate, prodrug, isotopic derivative, or tautomer thereof.

In some embodiments, the compound is of Formula (I-A-I-A-c):

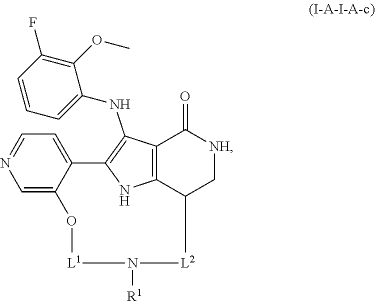

or a pharmaceutically acceptable salt, stereoisomer, solvate, prodrug, isotopic derivative, or tautomer thereof.

In some embodiments, the compound is of Formula (I-A-I-A-c′):

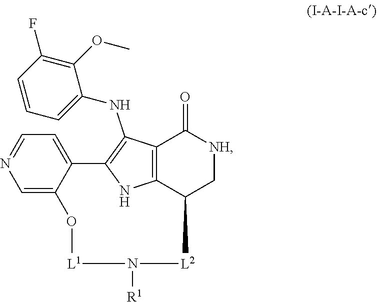

or a pharmaceutically acceptable salt, stereoisomer, solvate, prodrug, isotopic derivative, or tautomer thereof.

In some embodiments, the compound is of Formula (I-A-I-A-c″):

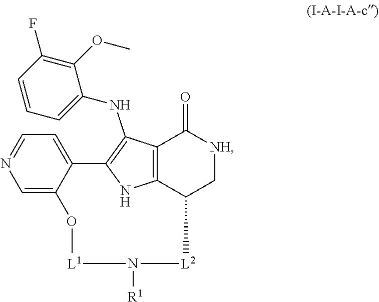

or a pharmaceutically acceptable salt, stereoisomer, solvate, prodrug, isotopic derivative, or tautomer thereof.

In some embodiments, the compound is of Formula (I-A-I-A-c-H):

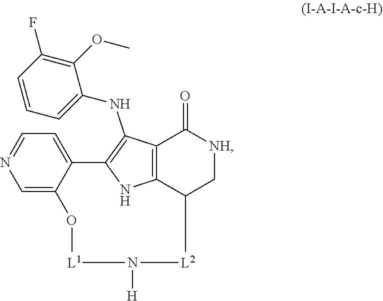

or a pharmaceutically acceptable salt, stereoisomer, solvate, prodrug, isotopic derivative, or tautomer thereof.

In some embodiments, the compound is of Formula (I-A-I-A-c-H′):

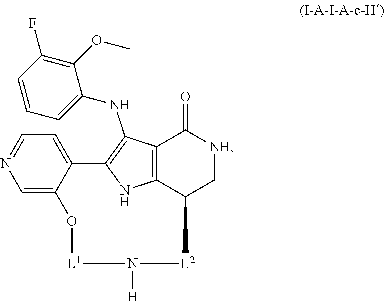

or a pharmaceutically acceptable salt, stereoisomer, solvate, prodrug, isotopic derivative, or tautomer thereof.

In some embodiments, the compound is of Formula (I-A-I-A-c-H″):

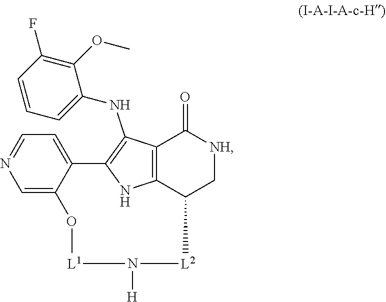

or a pharmaceutically acceptable salt, stereoisomer, solvate, prodrug, isotopic derivative, or tautomer thereof.

In some embodiments, the compound is of Formula (I-A-I-A-c-M):

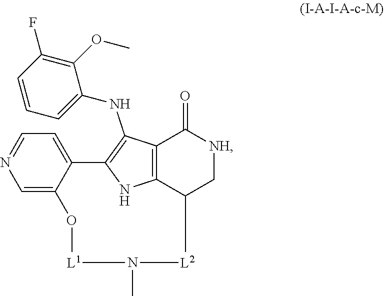

or a pharmaceutically acceptable salt, stereoisomer, solvate, prodrug, isotopic derivative, or tautomer thereof.

In some embodiments, the compound is of Formula (I-A-I-A-c-M′):

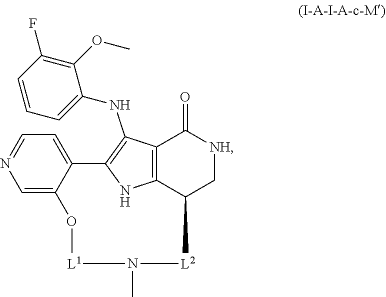

or a pharmaceutically acceptable salt, stereoisomer, solvate, prodrug, isotopic derivative, or tautomer thereof.

In some embodiments, the compound is of Formula (I-A-I-A-c-M″):

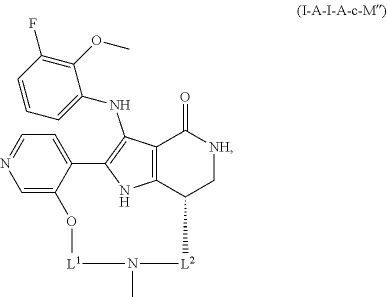

or a pharmaceutically acceptable salt, stereoisomer, solvate, prodrug, isotopic derivative, or tautomer thereof.

In some embodiments, the compound is of Formula (I-A-I-A-c-IP):

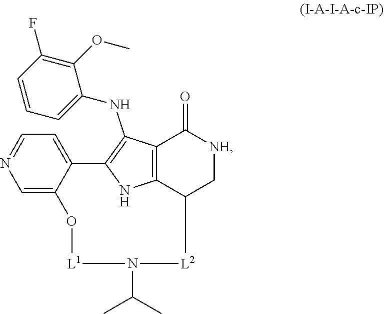

or a pharmaceutically acceptable salt, stereoisomer, solvate, prodrug, isotopic derivative, or tautomer thereof.

In some embodiments, the compound is of Formula (I-A-I-A-c-IP′):

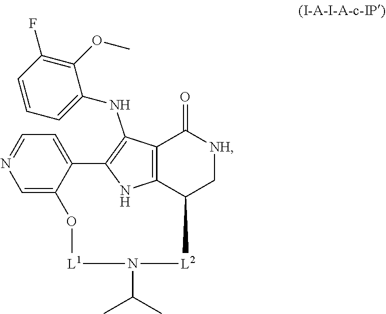

or a pharmaceutically acceptable salt, stereoisomer, solvate, prodrug, isotopic derivative, or tautomer thereof.

In some embodiments, the compound is of Formula (I-A-I-A-c-IP″):

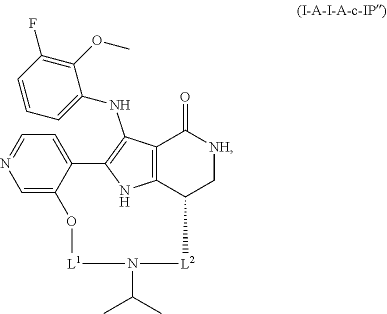

or a pharmaceutically acceptable salt, stereoisomer, solvate, prodrug, isotopic derivative, or tautomer thereof.

In some embodiments the compound is of Formula (I-A-I-A-c-Ar):

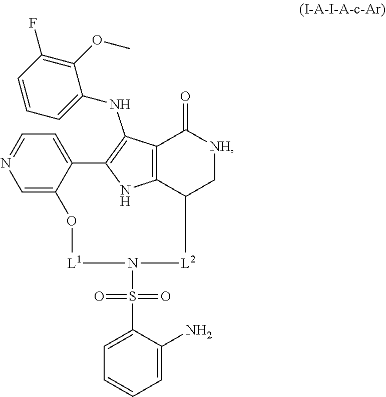

or a pharmaceutically acceptable salt, stereoisomer, solvate, prodrug, isotopic derivative, or tautomer thereof.

In some embodiments, the compound is of Formula (I-A-I-A-c-Ar′):

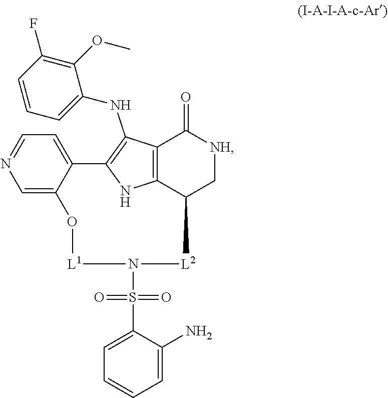

or a pharmaceutically acceptable salt, stereoisomer, solvate, prodrug, isotopic derivative, or tautomer thereof.

In some embodiments, the compound is of Formula (I-A-I-A-c-Ar″):

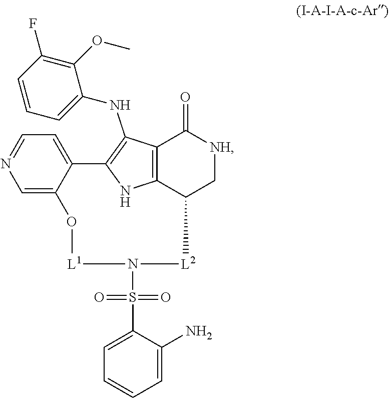

or a pharmaceutically acceptable salt, stereoisomer, solvate, prodrug, isotopic derivative, or tautomer thereof.

In some embodiments, the compound is of Formula (I-A-I-A-d):

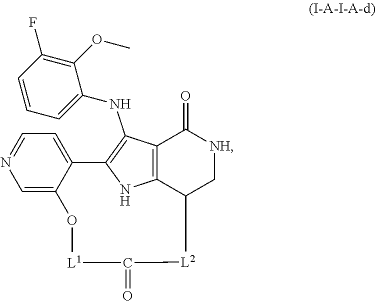

or a pharmaceutically acceptable salt, stereoisomer, solvate, prodrug, isotopic derivative, or tautomer thereof.

In some embodiments, the compound is of Formula (I-A-I-A-d′):

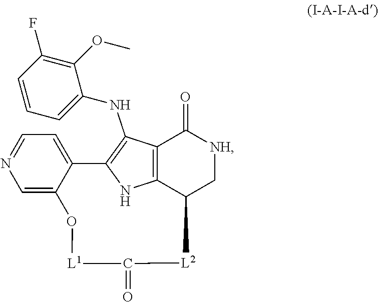

or a pharmaceutically acceptable salt, stereoisomer, solvate, prodrug, isotopic derivative, or tautomer thereof.

In some embodiments, the compound is of Formula (I-A-I-A-d″):

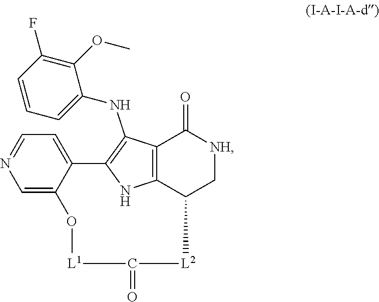

or a pharmaceutically acceptable salt, stereoisomer, solvate, prodrug, isotopic derivative, or tautomer thereof.

In some embodiments, the compound is of Formula (I-A-I-A-e):

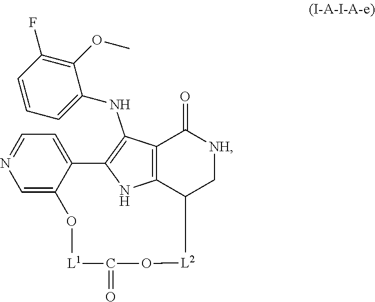

or a pharmaceutically acceptable salt, stereoisomer, solvate, prodrug, isotopic derivative, or tautomer thereof.

In some embodiments, the compound is of Formula (I-A-I-A-e′):

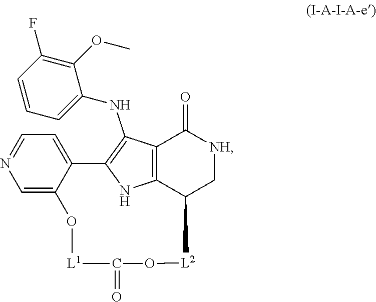

or a pharmaceutically acceptable salt, stereoisomer, solvate, prodrug, isotopic derivative, or tautomer thereof.

In some embodiments, the compound is of Formula (I-A-I-A-e″):

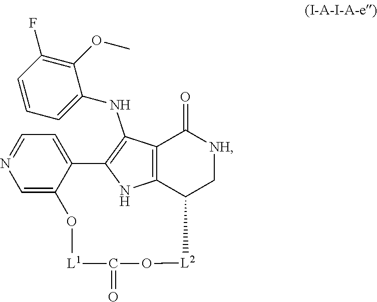

or a pharmaceutically acceptable salt, stereoisomer, solvate, prodrug, isotopic derivative, or tautomer thereof.

In some embodiments, the compound is of Formula (I-A-I-A-f):

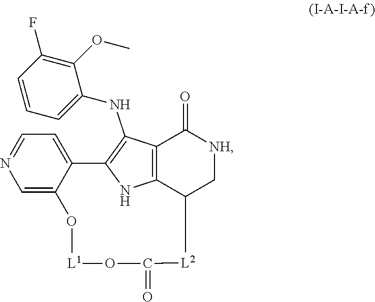

or a pharmaceutically acceptable salt, stereoisomer, solvate, prodrug, isotopic derivative, or tautomer thereof.

In some embodiments, the compound is of Formula (I-A-I-A-f′):

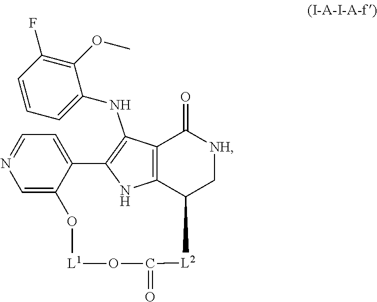

or a pharmaceutically acceptable salt, stereoisomer, solvate, prodrug, isotopic derivative, or tautomer thereof.

In some embodiments, the compound is of Formula (I-A-I-A-f′):

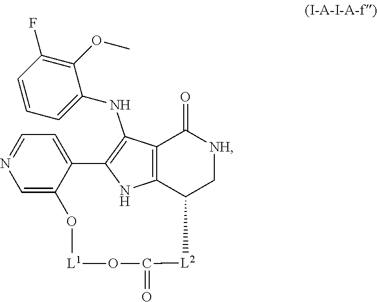

or a pharmaceutically acceptable salt, stereoisomer, solvate, prodrug, isotopic derivative, or tautomer thereof.

In some embodiments, the compound is of Formula (I-A-I-A-g):

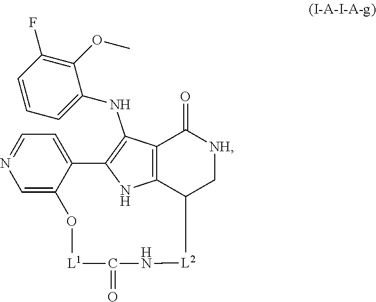

or a pharmaceutically acceptable salt, stereoisomer, solvate, prodrug, isotopic derivative, or tautomer thereof.

In some embodiments, the compound is of Formula (I-A-I-A-g′):

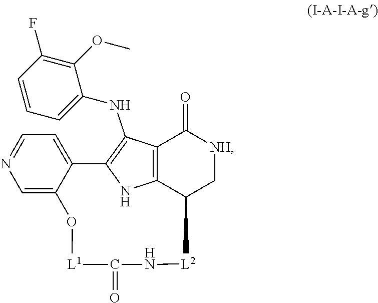

or a pharmaceutically acceptable salt, stereoisomer, solvate, prodrug, isotopic derivative, or tautomer thereof.

In some embodiments, the compound is of Formula (I-A-I-A-g″):

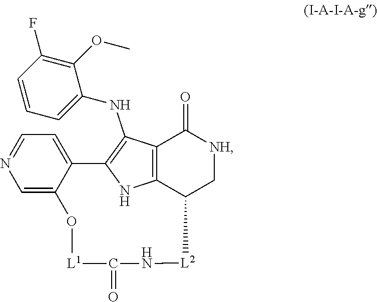

or a pharmaceutically acceptable salt, stereoisomer, solvate, prodrug, isotopic derivative, or tautomer thereof.

In some embodiments, the compound is of Formula (I-A-I-A-h):

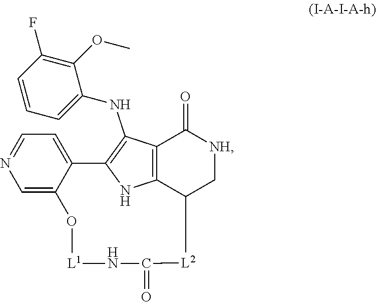

or a pharmaceutically acceptable salt, stereoisomer, solvate, prodrug, isotopic derivative, or tautomer thereof.

In some embodiments, the compound is of Formula (I-A-I-A-h′):

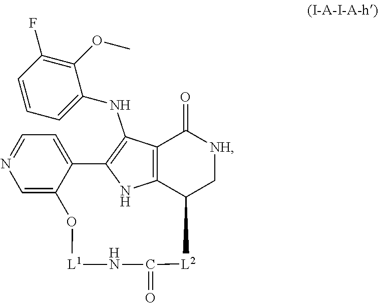

or a pharmaceutically acceptable salt, stereoisomer, solvate, prodrug, isotopic derivative, or tautomer thereof.

In some embodiments, the compound is of Formula (I-A-I-A-h″):

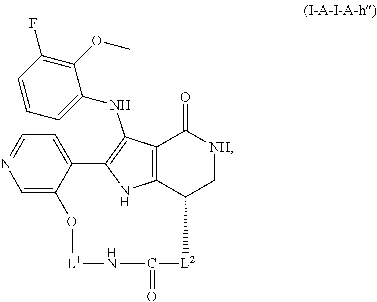

or a pharmaceutically acceptable salt, stereoisomer, solvate, prodrug, isotopic derivative, or tautomer thereof.

In some embodiments, the compound is of Formula (I-A-I-A-i):

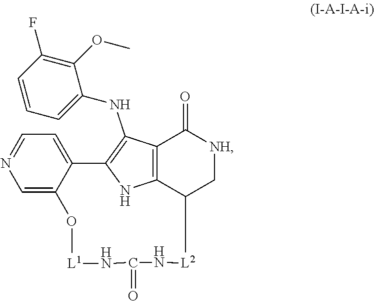

or a pharmaceutically acceptable salt, stereoisomer, solvate, prodrug, isotopic derivative, or tautomer thereof.

In some embodiments, the compound is of Formula (I-A-I-A-i′):

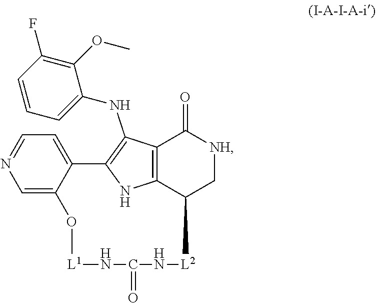

or a pharmaceutically acceptable salt, stereoisomer, solvate, prodrug, isotopic derivative, or tautomer thereof.

In some embodiments, the compound is of Formula (I-A-I-A-i″):

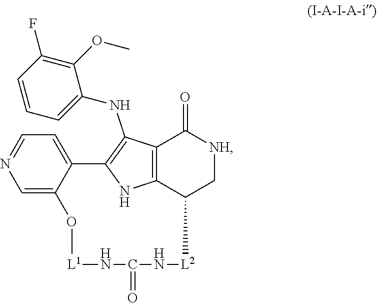

or a pharmaceutically acceptable salt, stereoisomer, solvate, prodrug, isotopic derivative, or tautomer thereof.

In some embodiments, the compound is of Formula (I-A-I-A-j):

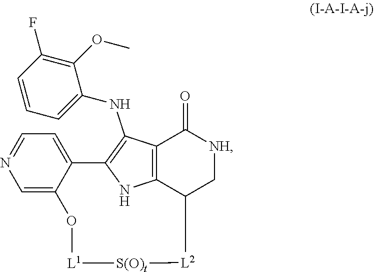

or a pharmaceutically acceptable salt, stereoisomer, solvate, prodrug, isotopic derivative, or tautomer thereof wherein t is 0, 1, or 2 and all other variables are as defined herein.

In some embodiments, the compound is of Formula (I-A-I-A-i′):

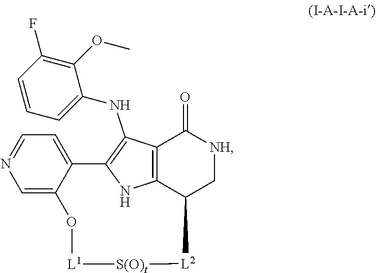

or a pharmaceutically acceptable salt, stereoisomer, solvate, prodrug, isotopic derivative, or tautomer thereof wherein t is 0, 1, or 2 and all other variables are as defined herein.

In some embodiments, the compound is of Formula (I-A-I-A-i″):

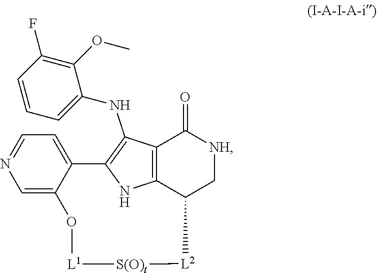

or a pharmaceutically acceptable salt, stereoisomer, solvate, prodrug, isotopic derivative, or tautomer thereof wherein t is 0, 1, or 2 and all other variables are as defined herein.

In some embodiments, the compound is of Formula (I-A-I-A-k):

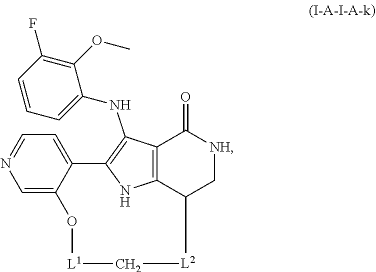

or a pharmaceutically acceptable salt, stereoisomer, solvate, prodrug, isotopic derivative, or tautomer thereof.

In some embodiments, the compound is of Formula (I-A-I-A-k′):

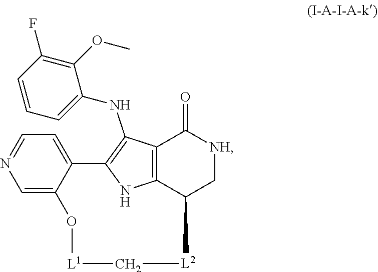

or a pharmaceutically acceptable salt, stereoisomer, solvate, prodrug, isotopic derivative, or tautomer thereof.

In some embodiments, the compound is of Formula (I-A-I-A-k″):

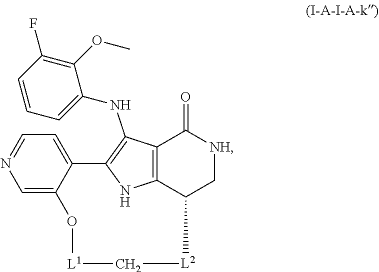

or a pharmaceutically acceptable salt, stereoisomer, solvate, prodrug, isotopic derivative, or tautomer thereof.

In some embodiments, the compound is of Formula (I-A-I-B-a):

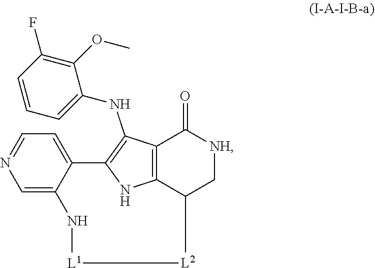

or a pharmaceutically acceptable salt, stereoisomer, solvate, pro rug, isotopic derivative, or tautomer thereof.

In some embodiments, the compound is of Formula (I-A-I-B-a′):

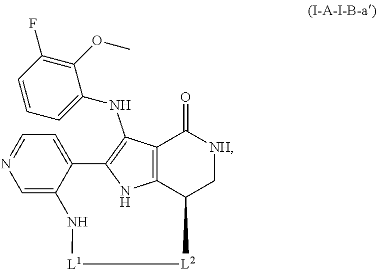

or a pharmaceutically acceptable salt, stereoisomer, solvate, prodrug, isotopic derivative, or tautomer thereof.

In some embodiments, the compound is of Formula (I-A-I-B-1-a″):

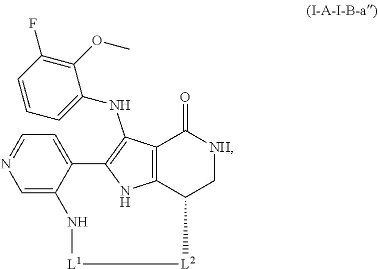

or a pharmaceutically acceptable salt, stereoisomer, solvate, prodrug, isotopic derivative, or tautomer thereof.

In some embodiments, the compound is of Formula (I-A-I-B-b):

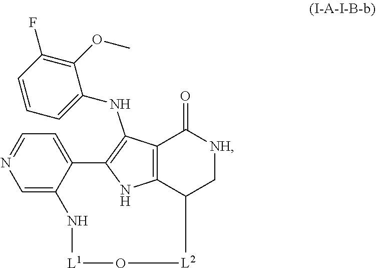

or a pharmaceutically acceptable salt, stereoisomer, solvate, prodrug, isotopic derivative, or tautomer thereof.

In some embodiments, the compound is of Formula (I-A-I-B-b′):

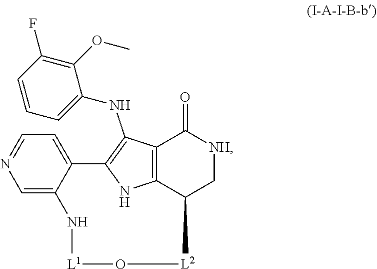

or a pharmaceutically acceptable salt, stereoisomer, solvate, prodrug, isotopic derivative, or tautomer thereof.

In some embodiments, the compound is of Formula (I-A-I-B-b″):

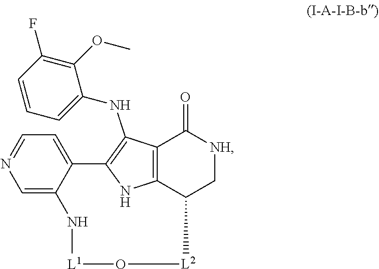

or a pharmaceutically acceptable salt, stereoisomer, solvate, prodrug, isotopic derivative, or tautomer thereof.

In some embodiments, the compound is of Formula (I-A-I-B-c):

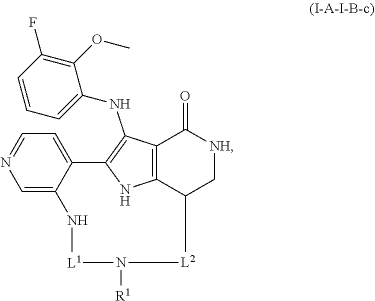

or a pharmaceutically acceptable salt, stereoisomer, solvate, prodrug, isotopic derivative, or tautomer thereof.

In some embodiments, the compound is of Formula (I-A-I-B-c′):

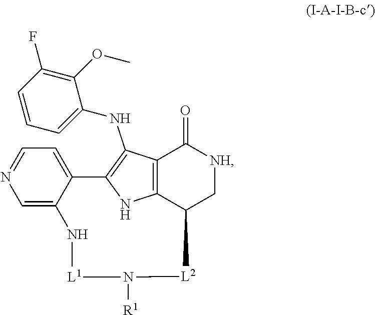

or a pharmaceutically acceptable salt, stereoisomer, solvate, prodrug, isotopic derivative, or tautomer thereof.

In some embodiments, the compound is of Formula (I-A-I-B-c″):

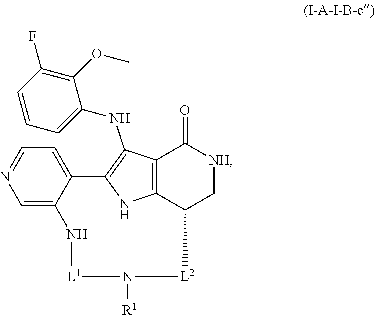

or a pharmaceutically acceptable salt, stereoisomer, solvate, prodrug, isotopic derivative, or tautomer thereof.

In some embodiments, the compound is of Formula (I-A-I-B-c-H):

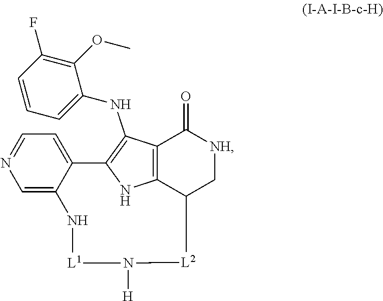

or a pharmaceutically acceptable salt, stereoisomer, solvate, prodrug, isotopic derivative, or tautomer thereof.

In some embodiments, the compound is of Formula (I-A-I-B-c-H′):

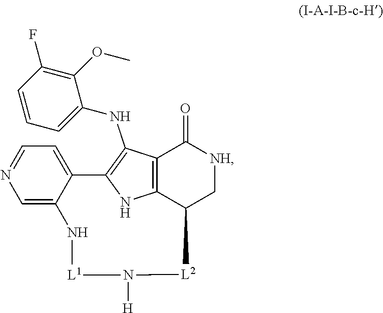

or a pharmaceutically acceptable salt, stereoisomer, solvate, prodrug, isotopic derivative, or tautomer thereof.

In some embodiments, the compound is of Formula (I-A-I-B-c-H″):

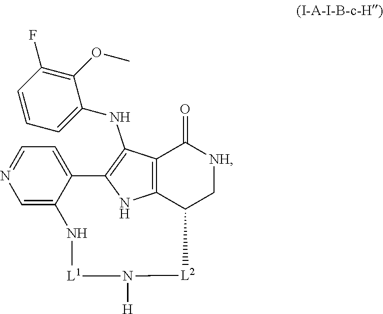

or a pharmaceutically acceptable salt, stereoisomer, solvate, prodrug, isotopic derivative, or tautomer thereof.

In some embodiments, the compound is of Formula (I-A-I-B-c-M):

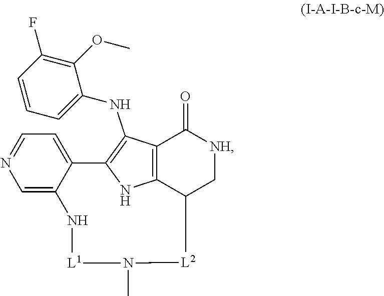

or a pharmaceutically acceptable salt, stereoisomer, solvate, prodrug, isotopic derivative, or tautomer thereof.

In some embodiments, the compound is of Formula (I-A-I-B-c-M′):

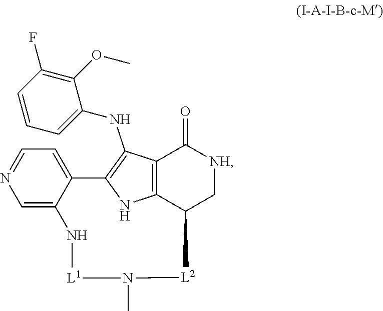

or a pharmaceutically acceptable salt, stereoisomer, solvate, prodrug, isotopic derivative, or tautomer thereof.

In some embodiments, the compound is of Formula (I-A-I-B-c-M″):

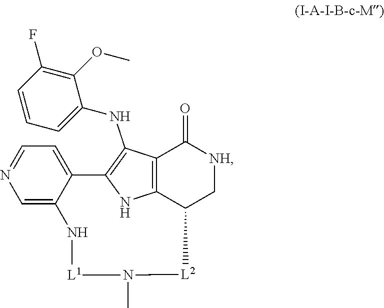

or a pharmaceutically acceptable salt, stereoisomer, solvate, prodrug, isotopic derivative, or tautomer thereof.

In some embodiments, the compound is of Formula (I-A-I-B-d):

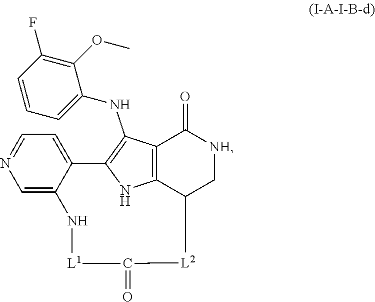

or a pharmaceutically acceptable salt, stereoisomer, solvate, prodrug, isotopic derivative, or tautomer thereof.

In some embodiments, the compound is of Formula (I-A-I-B-d′):

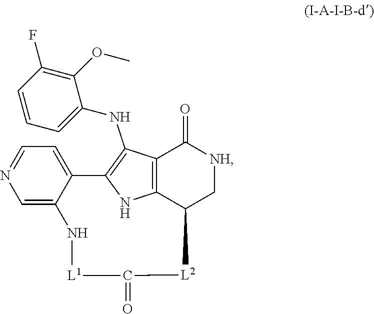

or a pharmaceutically acceptable salt, stereoisomer, solvate, prodrug, isotopic derivative, or tautomer thereof.

In some embodiments, the compound is of Formula (I-A-I-B-d″):

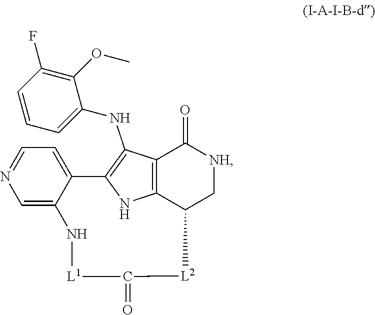

or a pharmaceutically acceptable salt, stereoisomer, solvate, prodrug, isotopic derivative, or tautomer thereof.

In some embodiments, the compound is of Formula (I-A-I-B-e):

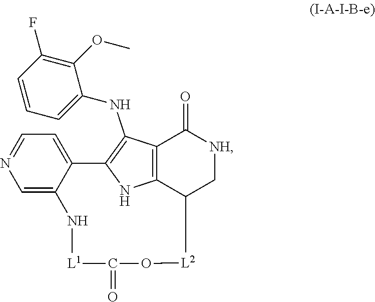

or a pharmaceutically acceptable salt, stereoisomer, solvate, prodrug, isotopic derivative, or tautomer thereof.

In some embodiments, the compound is of Formula (I-A-I-B-e′):

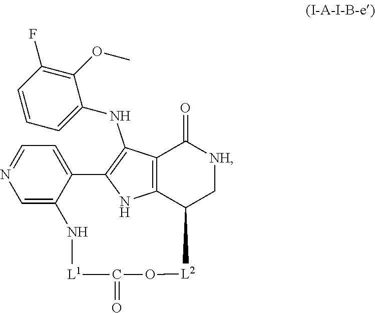

or a pharmaceutically acceptable salt, stereoisomer, solvate, prodrug, isotopic derivative, or tautomer thereof.

In some embodiments, the compound is of Formula (I-A-I-B-e″):

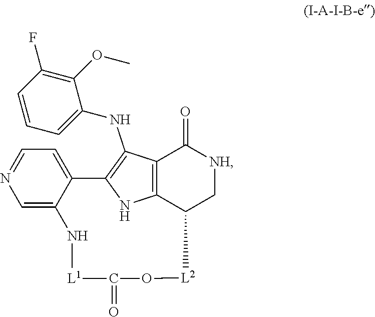

or a pharmaceutically acceptable salt, stereoisomer, solvate, prodrug, isotopic derivative, or tautomer thereof.

In some embodiments, the compound is of Formula (I-A-I-B-f):

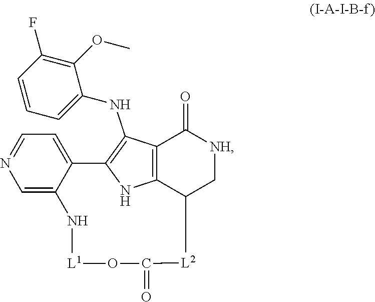

or a pharmaceutically acceptable salt, stereoisomer, solvate, prodrug, isotopic derivative, or tautomer thereof.

In some embodiments, the compound is of Formula (I-A-I-B-f′):

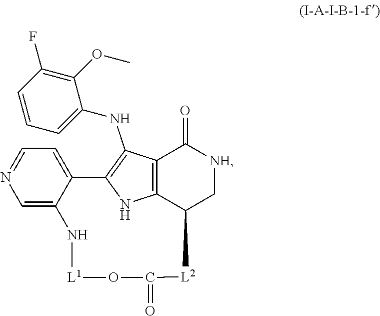

or a pharmaceutically acceptable salt, stereoisomer, solvate, prodrug, isotopic derivative, or tautomer thereof.

In some embodiments, the compound is of Formula (I-A-I-B-f′):

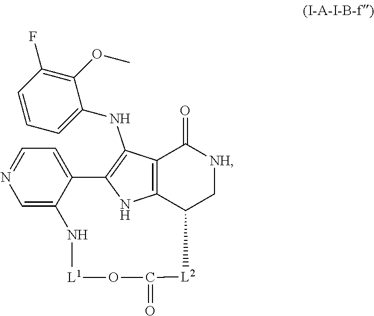

or a pharmaceutically acceptable salt, stereoisomer, solvate, prodrug, isotopic derivative, or tautomer thereof.

In some embodiments, the compound is of Formula (I-A-I-B-g):

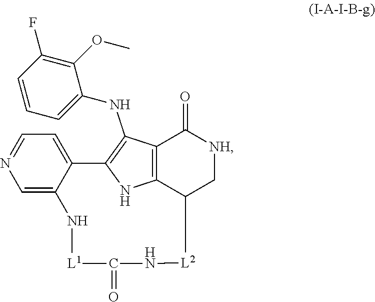

or a pharmaceutically acceptable salt, stereoisomer, solvate, prodrug, isotopic derivative, or tautomer thereof.

In some embodiments, the compound is of Formula (I-A-I-B-g′):

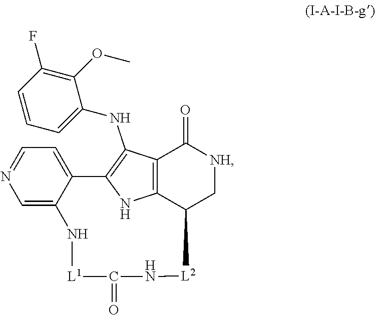

or a pharmaceutically acceptable salt, stereoisomer, solvate, prodrug, isotopic derivative, or tautomer thereof.

In some embodiments, the compound is of Formula (I-A-I-B-g″):

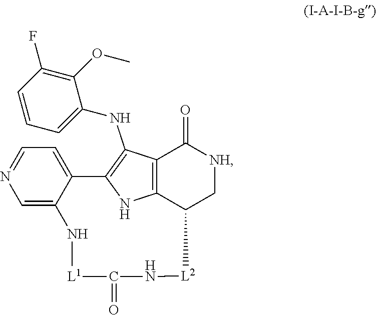

or a pharmaceutically acceptable salt, stereoisomer, solvate, prodrug, isotopic derivative, or tautomer thereof.

In some embodiments, the compound is of Formula (I-A-I-B-h):

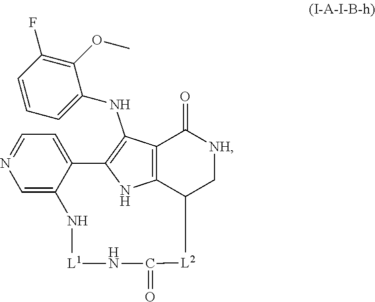

or a pharmaceutically acceptable salt, stereoisomer, solvate, prodrug, isotopic derivative, or tautomer thereof.

In some embodiments, the compound is of Formula (I-A-I-B-h′):

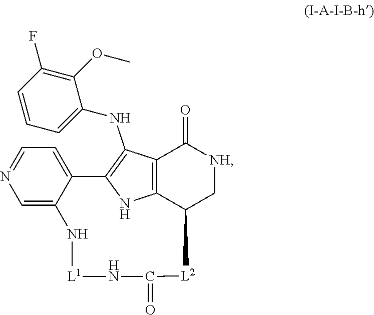

or a pharmaceutically acceptable salt, stereoisomer, solvate, prodrug, isotopic derivative, or tautomer thereof.

In some embodiments, the compound is of Formula (I-A-I-B-1-h″):

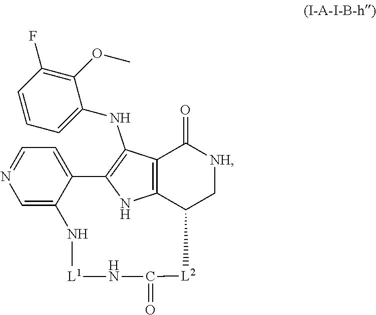

or a pharmaceutically acceptable salt, stereoisomer, solvate, prodrug, isotopic derivative, or tautomer thereof.

In some embodiments, the compound is of Formula (I-A-I-B-i):

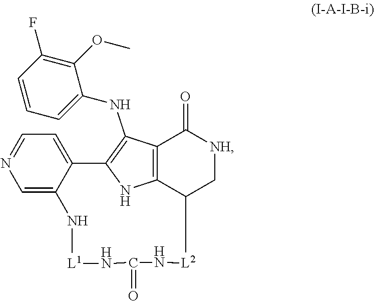

or a pharmaceutically acceptable salt, stereoisomer, solvate, prodrug, isotopic derivative, or tautomer thereof.

In some embodiments, the compound is of Formula (I-A-I-B-i′):

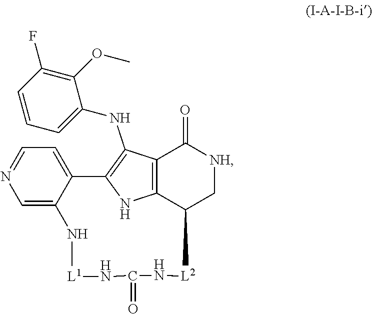

or a pharmaceutically acceptable salt, stereoisomer, solvate, prodrug, isotopic derivative, or tautomer thereof.

In some embodiments, the compound is of Formula (I-A-I-B-i″):

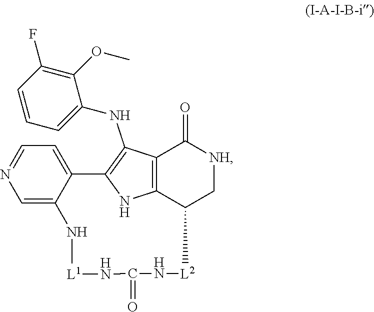

or a pharmaceutically acceptable salt, stereoisomer, solvate, prodrug, isotopic derivative, or tautomer thereof.

In some embodiments, the compound is of Formula (I-A-I-B-j):

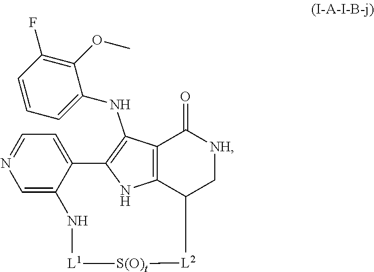

or a pharmaceutically acceptable salt, stereoisomer, solvate, prodrug, isotopic derivative, or tautomer thereof wherein t is 0, 1, or 2 and all other variables are as defined herein.

In some embodiments, the compound is of Formula (I-A-I-B-i′):

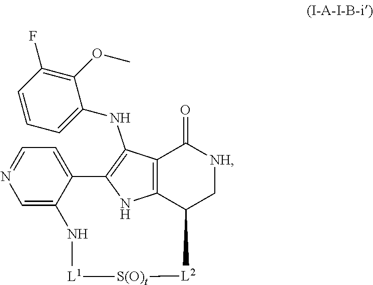

or a pharmaceutically acceptable salt, stereoisomer, solvate, prodrug, isotopic derivative, or tautomer thereof wherein t is 0, 1, or 2 and all other variables are as defined herein.

In some embodiments, the compound is of Formula (I-A-I-B-i″):

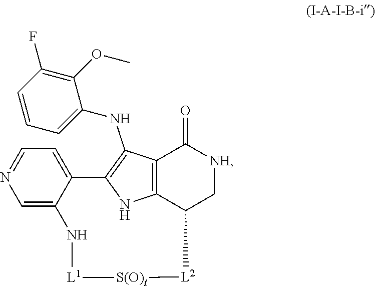

or a pharmaceutically acceptable salt, stereoisomer, solvate, prodrug, isotopic derivative, or tautomer thereof wherein t is 0, 1, or 2 and all other variables are as defined herein.

In some embodiments, the compound is of Formula (I-A-I-B-k):

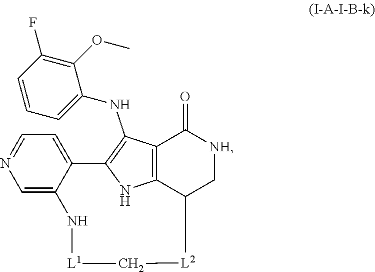

or a pharmaceutically acceptable salt, stereoisomer, solvate, prodrug, isotopic derivative, or tautomer thereof.

In some embodiments, the compound is of Formula (I-A-I-B-k′):

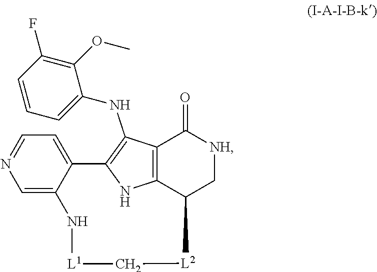

or a pharmaceutically acceptable salt, stereoisomer, solvate, prodrug, isotopic derivative, or tautomer thereof.

In some embodiments, the compound is of Formula (I-A-I-B-k″):

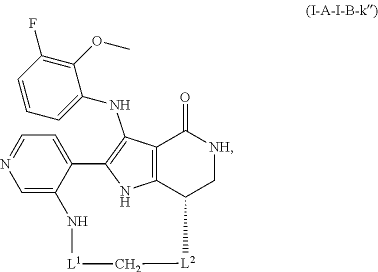

or a pharmaceutically acceptable salt, stereoisomer, solvate, prodrug, isotopic derivative, or tautomer thereof.

In some embodiments, the compound is of Formula (I-A-I-A-a-1):

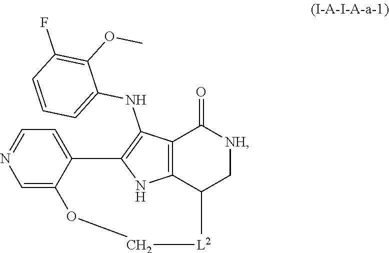

or a pharmaceutically acceptable salt, stereoisomer, solvate, prodrug, isotopic derivative, or tautomer thereof.

In some embodiments, the compound is of Formula (I-A-I-A-a-1′) or (I-A-I-A-a-1″):

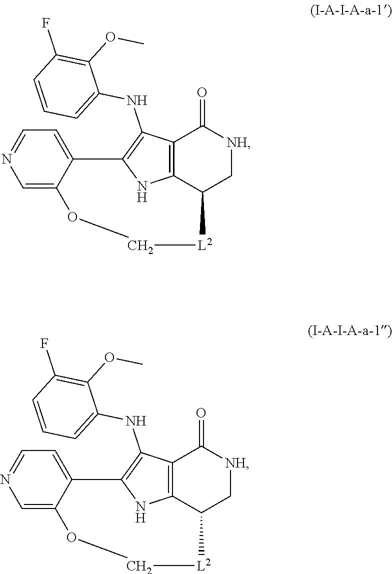

or a pharmaceutically acceptable salt, stereoisomer, solvate, prodrug, isotopic derivative, or tautomer thereof.

In some embodiments, the compound is of Formula (I-A-I-A-a-2):

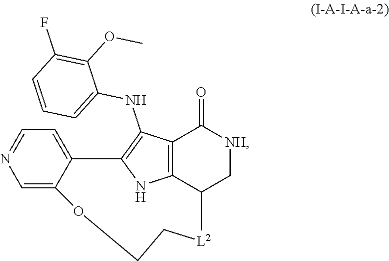

or a pharmaceutically acceptable salt, stereoisomer, solvate, prodrug, isotopic derivative, or tautomer thereof.

In some embodiments, the compound is of Formula (I-A-I-A-a-2′) or (I-A-I-A-a-2″):

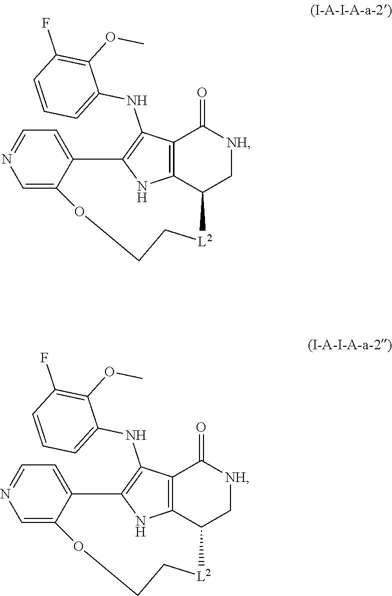

or a pharmaceutically acceptable salt, stereoisomer, solvate, prodrug, isotopic derivative, or tautomer thereof.

In some embodiments, the compound is of Formula (I-A-I-A-a-3):

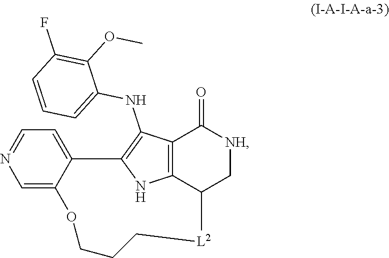

or a pharmaceutically acceptable salt, stereoisomer, solvate, prodrug, isotopic derivative, or tautomer thereof.

In some embodiments, the compound is of Formula (I-A-I-A-a-3′) or (I-A-I-A-a-3″):

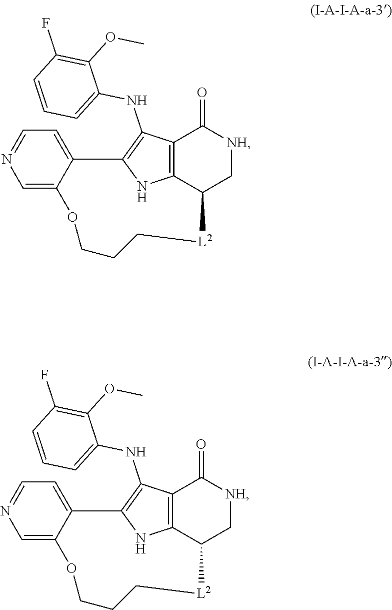

or a pharmaceutically acceptable salt, stereoisomer, solvate, prodrug, isotopic derivative, or tautomer thereof.

In some embodiments, the compound is of Formula (I-A-I-A-a-4):

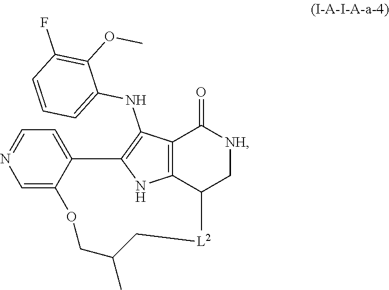

or a pharmaceutically acceptable salt, stereoisomer, solvate, prodrug, isotopic derivative, or tautomer thereof.

In some embodiments, the compound is of Formula (I-A-I-A-a-4′) or (I-A-I-A-a-4″):

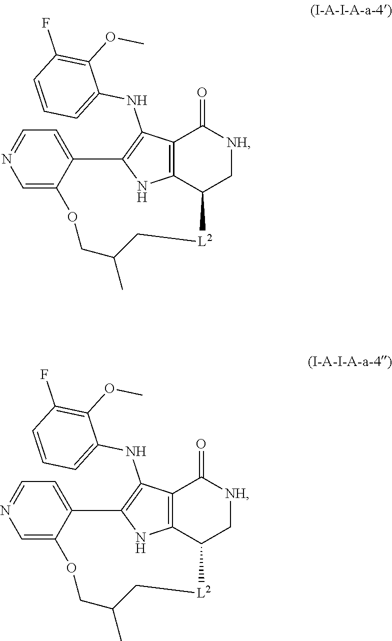

or a pharmaceutically acceptable salt, stereoisomer, solvate, prodrug, isotopic derivative, or tautomer thereof.

In some embodiments, the compound is of Formula (I-A-I-A-a-5):

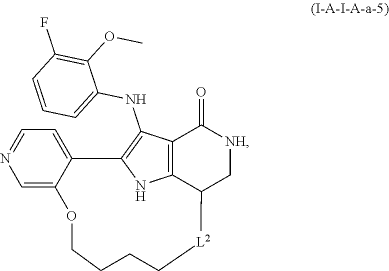

or a pharmaceutically acceptable salt, stereoisomer, solvate, prodrug, isotopic derivative, or tautomer thereof.

In some embodiments, the compound is of Formula (I-A-I-A-a-5′) or (I-A-I-A-a-5″):

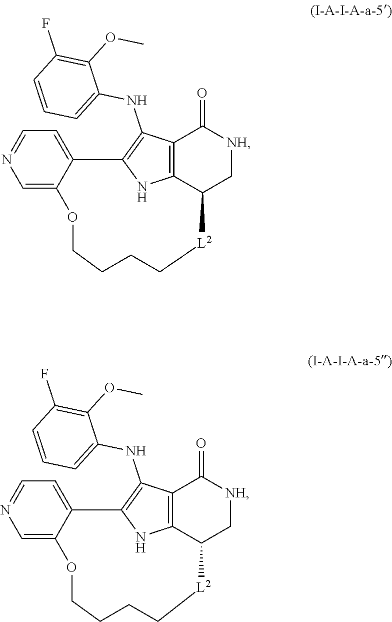

or a pharmaceutically acceptable salt, stereoisomer, solvate, prodrug, isotopic derivative, or tautomer thereof.

In some embodiments, the compound is of Formula (I-A-I-A-a-6):

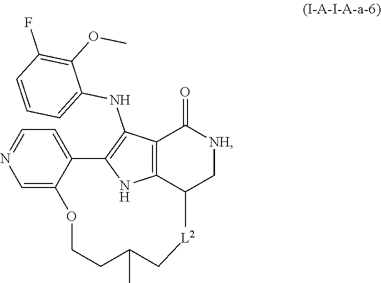

or a pharmaceutically acceptable salt, stereoisomer, solvate, prodrug, isotopic derivative, or tautomer thereof.

In some embodiments, the compound is of Formula (I-A-I-A-a-6′) or (I-A-I-A-a-6″):

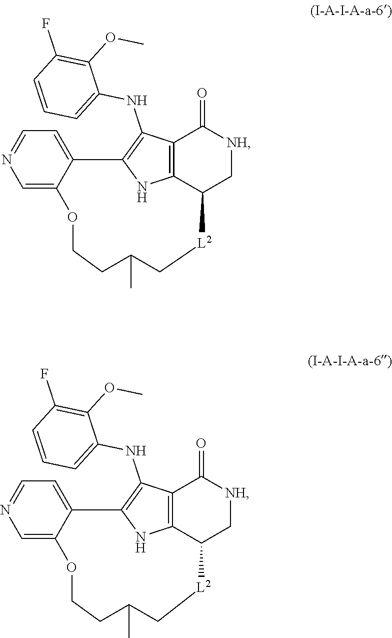

or a pharmaceutically acceptable salt, stereoisomer, solvate, prodrug, isotopic derivative, or tautomer thereof.

In some embodiments, the compound is of Formula (I-A-I-A-a-7):

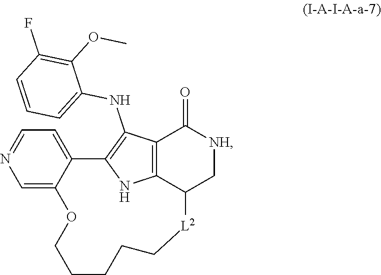

or a pharmaceutically acceptable salt, stereoisomer, solvate, prodrug, isotopic derivative, or tautomer thereof.

In some embodiments, the compound is of Formula (I-A-I-A-a-7′) or (I-A-I-A-a-7″):

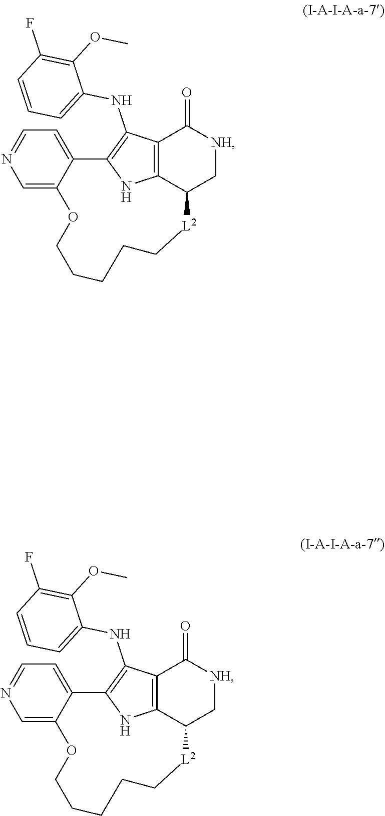

or a pharmaceutically acceptable salt, stereoisomer, solvate, prodrug, isotopic derivative, or tautomer thereof.

In some embodiments, the compound is of Formula (I-A-I-A-a-8):

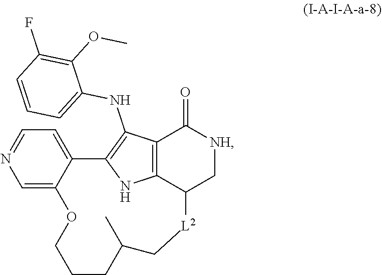

or a pharmaceutically acceptable salt, stereoisomer, solvate, prodrug, isotopic derivative, or tautomer thereof.

In some embodiments, the compound is of Formula (I-A-I-A-a-8′) or (I-A-I-A-a-8″):

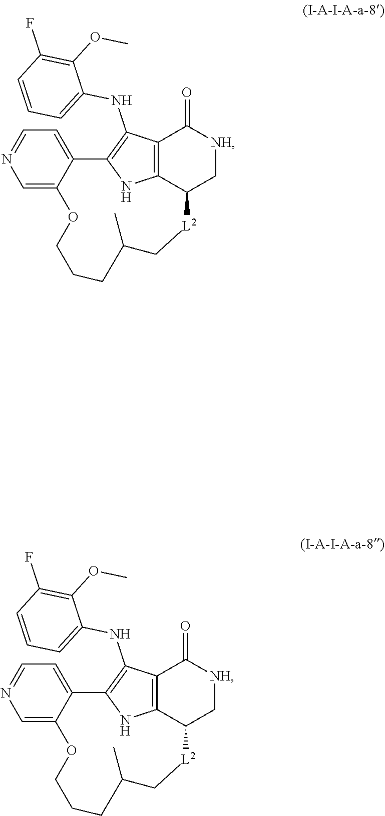

or a pharmaceutically acceptable salt, stereoisomer, solvate, prodrug, isotopic derivative, or tautomer thereof.

In some embodiments, the compound is of Formula (I-A-I-A-a-9):

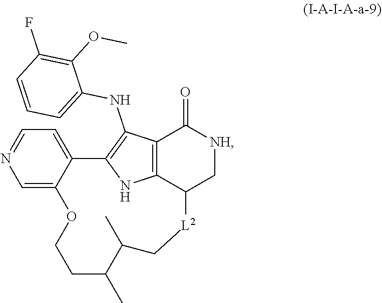

or a pharmaceutically acceptable salt, stereoisomer, solvate, prodrug, isotopic derivative, or tautomer thereof.

In some embodiments, the compound is of Formula (I-A-I-A-a-9′) or (I-A-I-A-a-9″):

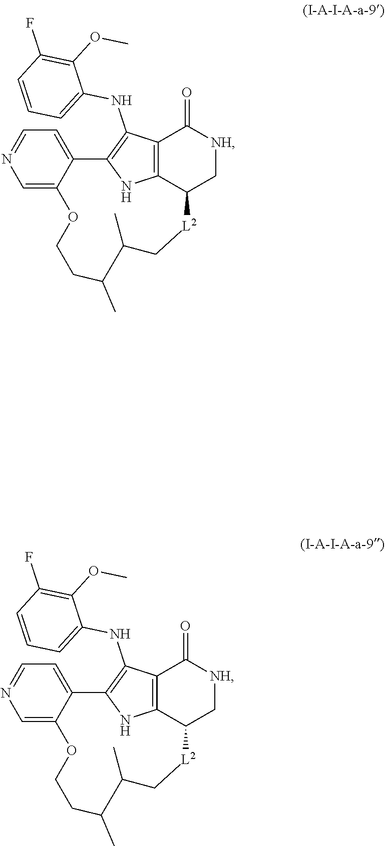

or a pharmaceutically acceptable salt, stereoisomer, solvate, prodrug, isotopic derivative, or tautomer thereof.

In some embodiments, the compound is of Formula (I-A-I-A-a-10):

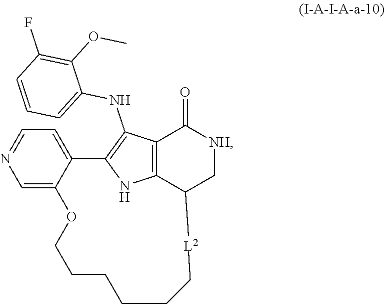

or a pharmaceutically acceptable salt, stereoisomer, solvate, prodrug, isotopic derivative, or tautomer thereof.

In some embodiments, the compound is of Formula (I-A-I-A-a-10′) or (I-A-I-A-a-10″):

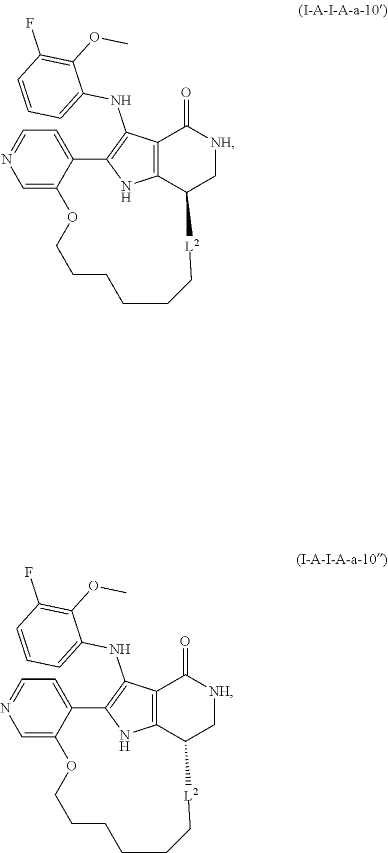

or a pharmaceutically acceptable salt, stereoisomer, solvate, prodrug, isotopic derivative, or tautomer thereof.

In some embodiments, the compound is of Formula (I-A-I-A-a-11):

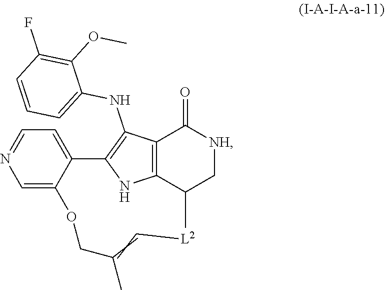

or a pharmaceutically acceptable salt, stereoisomer, solvate, prodrug, isotopic derivative, or tautomer thereof.

In some embodiments, the compound is of Formula (E-I-A-I-A-a-11) or (Z-I-A-I-A-a-11):

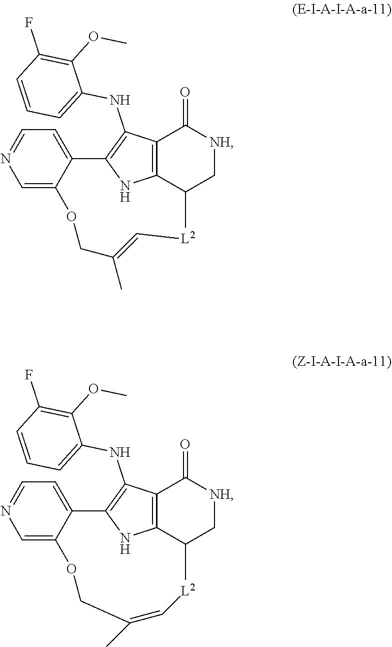

or a pharmaceutically acceptable salt, stereoisomer, solvate, prodrug, isotopic derivative, or tautomer thereof.

In some embodiments, the compound is of Formula (E-I-A-I-A-a-11′), (Z-I-A-I-A-a-11′), (E-I-A-I-A-a-11″), or (Z-I-A-I-A-a-11″):

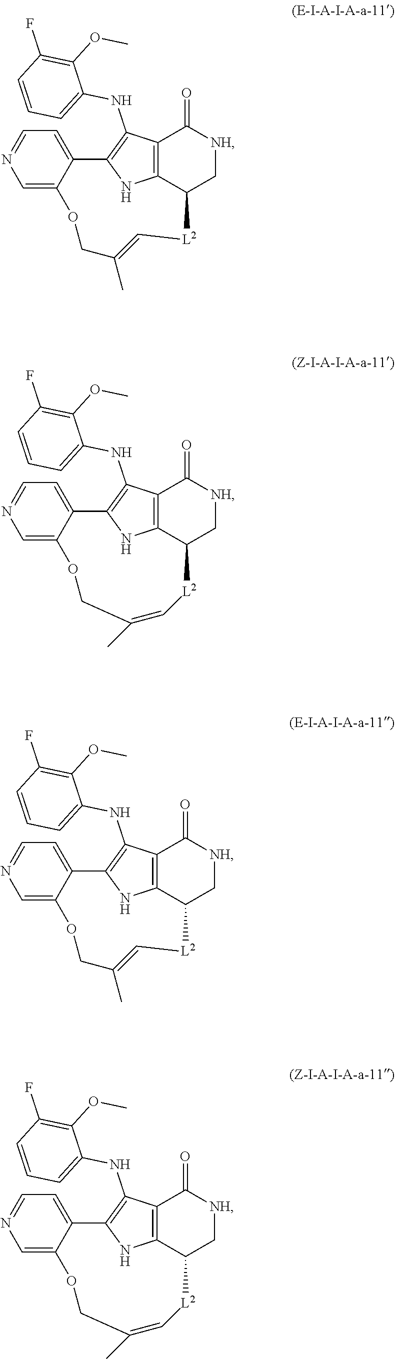

or a pharmaceutically acceptable salt, stereoisomer, solvate, prodrug, isotopic derivative, or tautomer thereof.

In some embodiments, the compound is of Formula (I-A-I-A-a-12):

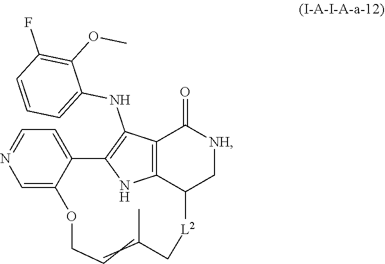

or a pharmaceutically acceptable salt, stereoisomer, solvate, prodrug, isotopic derivative, or tautomer thereof.

In some embodiments, the compound is of Formula (E-I-A-I-A-a-12) or (Z-I-A-I-A-a-12):

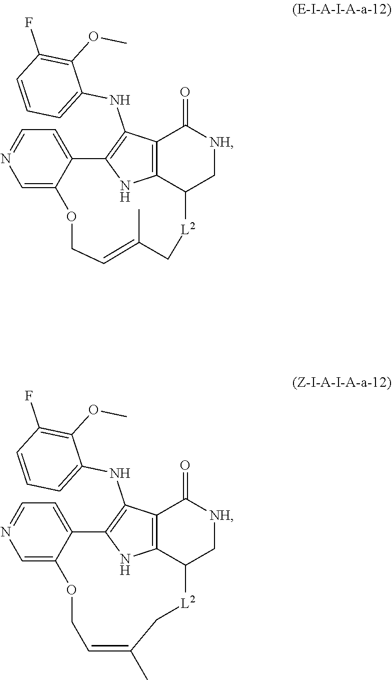

or a pharmaceutically acceptable salt, stereoisomer, solvate, prodrug, isotopic derivative, or tautomer thereof.

In some embodiments, the compound is of Formula (E-I-A-I-A-a-12′), (E-I-A-I-A-a-12″), (Z-I-A-I-A-a-12′), or (Z-I-A-I-A-a-12″):

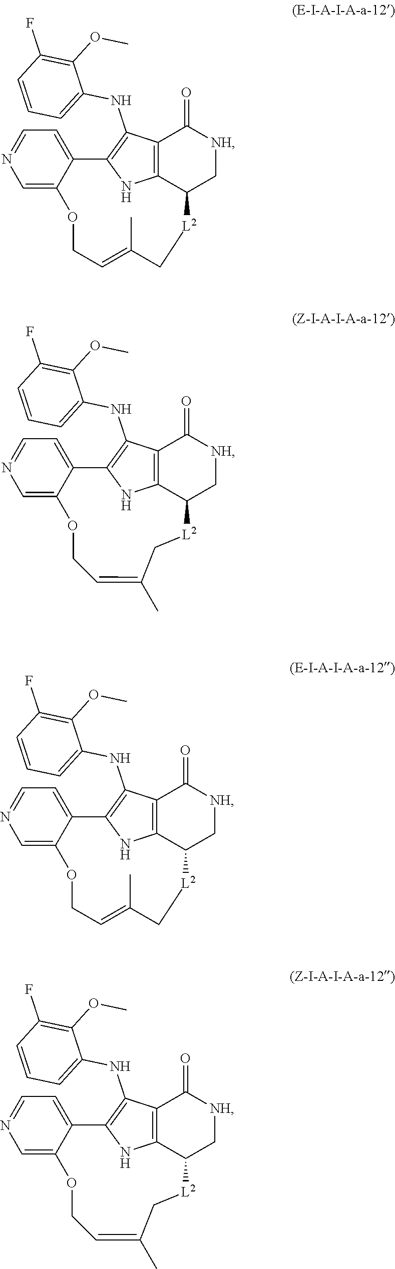

or a pharmaceutically acceptable salt, stereoisomer, solvate, prodrug, isotopic derivative, or tautomer thereof.

In some embodiments, the compound is of Formula (I-A-I-A-a-13):

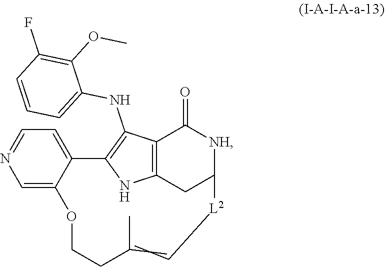

or a pharmaceutically acceptable salt, stereoisomer, solvate, prodrug, isotopic derivative, or tautomer thereof.

In some embodiments, the compound is of Formula (E-I-A-I-A-a-13) or (Z-I-A-I-A-a-13):

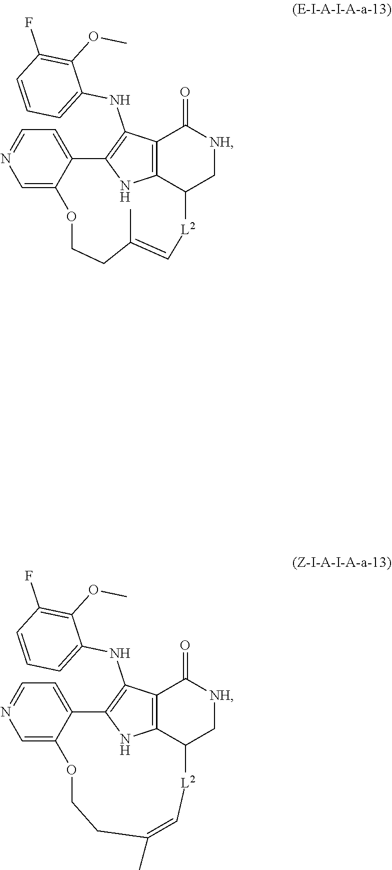

or a pharmaceutically acceptable salt, stereoisomer, solvate, prodrug, isotopic derivative, or tautomer thereof.

In some embodiments, the compound is of Formula (E-I-A-I-A-a-13′), (E-I-A-I-A-a-13″), (7-T-A-T-A-a-13′) or (7-T-A-I-A-a-l 3″)

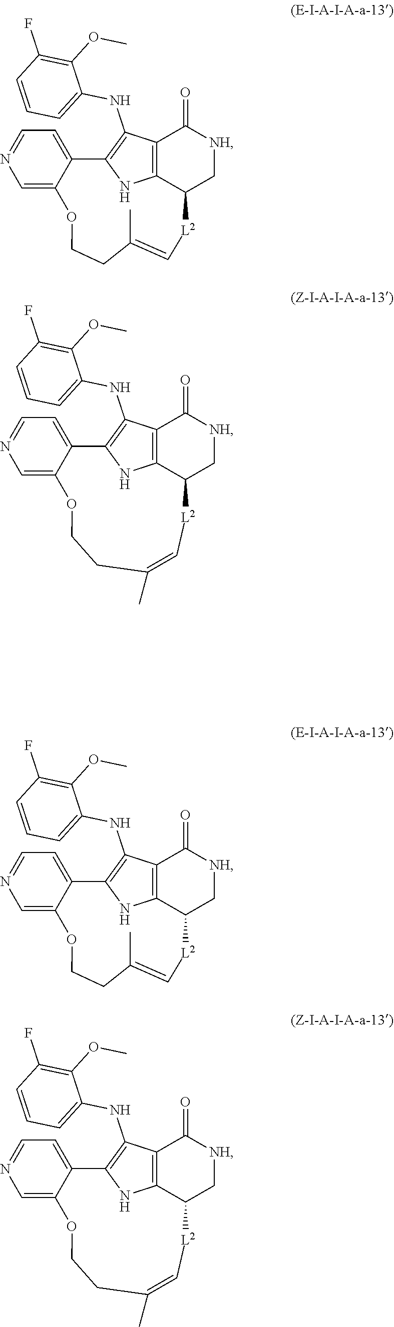

or a pharmaceutically acceptable salt, stereoisomer, solvate, prodrug, isotopic derivative, or tautomer thereof.

In some embodiments, the compound is of Formula (I-A-I-A-a-14):

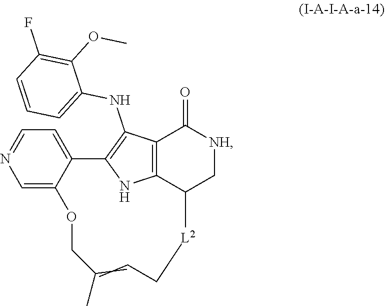

or a pharmaceutically acceptable salt, stereoisomer, solvate, prodrug, isotopic derivative, or tautomer thereof.

In some embodiments, the compound is of Formula (E-I-A-I-A-a-14) or (Z-I-A-I-A-a-14):

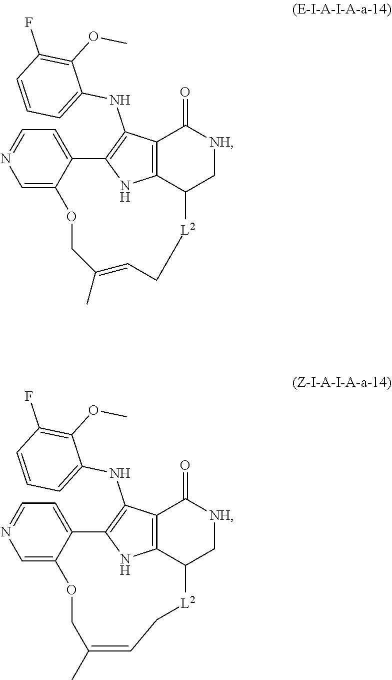

or a pharmaceutically acceptable salt, stereoisomer, solvate, prodrug, isotopic derivative, or tautomer thereof.

In some embodiments, the compound is of Formula (E-I-A-I-A-a-14′), (E-I-A-I-A-a-14″), (Z-I-A-I-A-a-14′), or (Z-I-A-I-A-a-14″):

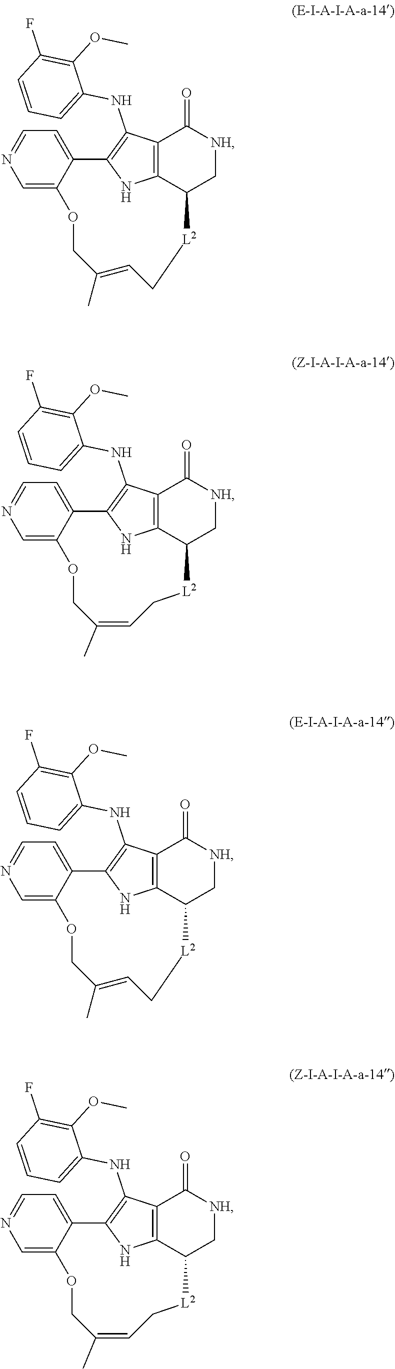

or a pharmaceutically acceptable salt, stereoisomer, solvate, prodrug, isotopic derivative, or tautomer thereof.

In some embodiments, the compound is of Formula (I-A-I-A-a-15):

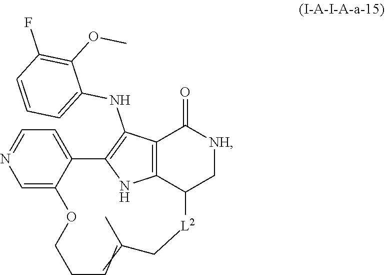

or a pharmaceutically acceptable salt, stereoisomer, solvate, prodrug, isotopic derivative, or tautomer thereof.

In some embodiments, the compound is of Formula (E-I-A-I-A-a-15) or (Z-I-A-I-A-a-15):

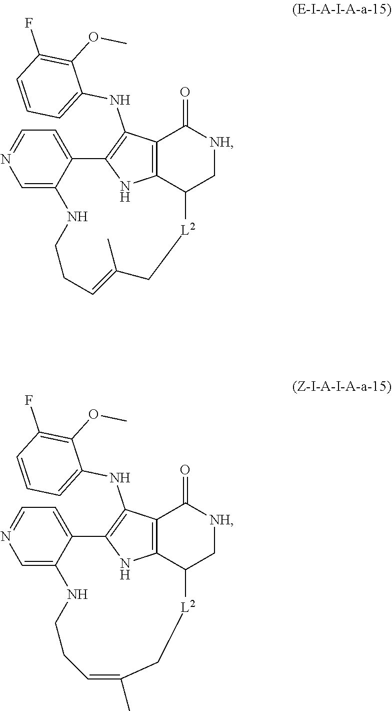

or a pharmaceutically acceptable salt, stereoisomer, solvate, prodrug, isotopic derivative, or tautomer thereof.

In some embodiments, the compound is of Formula (E-I-A-I-A-a-15′), (E-I-A-I-A-a-15″), (Z-I-A-I-A-a-15′), or (Z-I-A-I-A-a-15″):

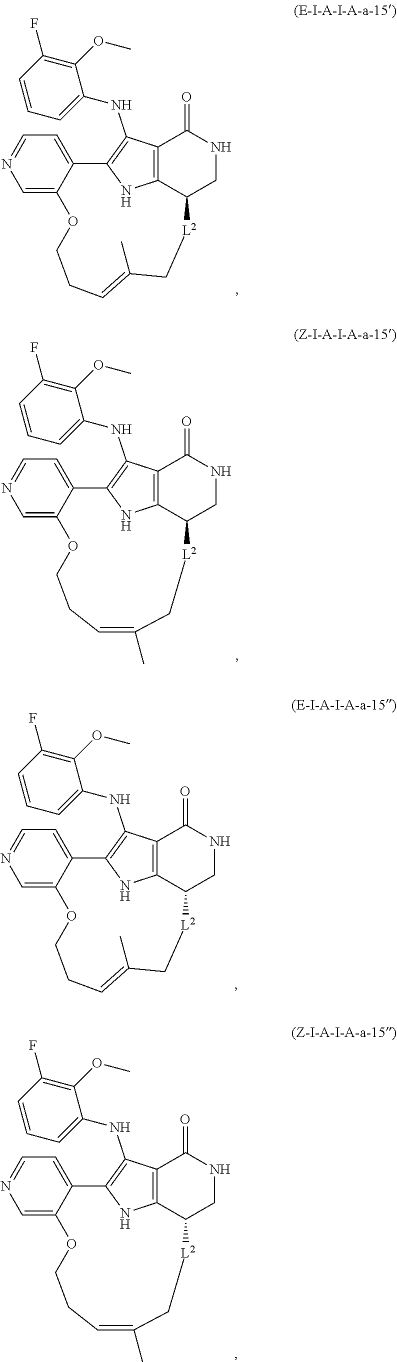

or a pharmaceutically acceptable salt, stereoisomer, solvate, prodrug, isotopic derivative, or tautomer thereof.

In some embodiments, the compound is of Formula (I-A-I-A-a-16):

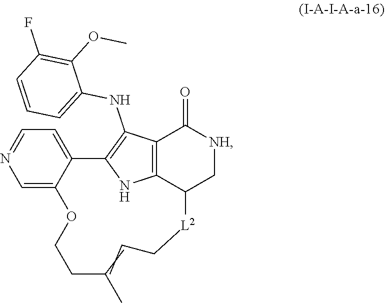

or a pharmaceutically acceptable salt, stereoisomer, solvate, prodrug, isotopic derivative, or tautomer thereof.

In some embodiments, the compound is of Formula (E-I-A-I-A-a-16) or (Z-I-A-I-A-a-16):

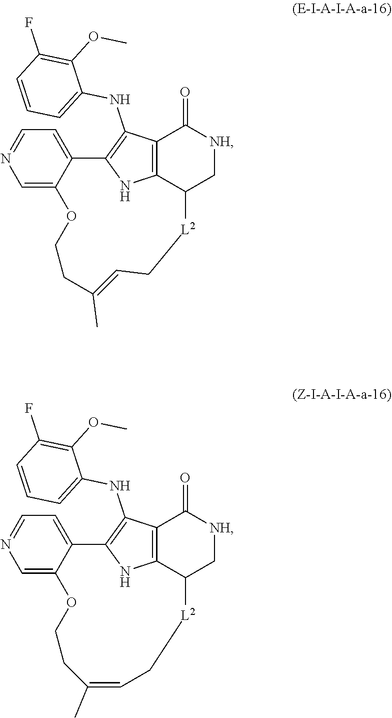

or a pharmaceutically acceptable salt, stereoisomer, solvate, prodrug, isotopic derivative, or tautomer thereof.

In some embodiments, the compound is of Formula (E-I-A-I-A-a-16′), (E-I-A-I-A-a-16″), (Z-I-A-I-A-a-16′), or (Z-I-A-I-A-a-16″):

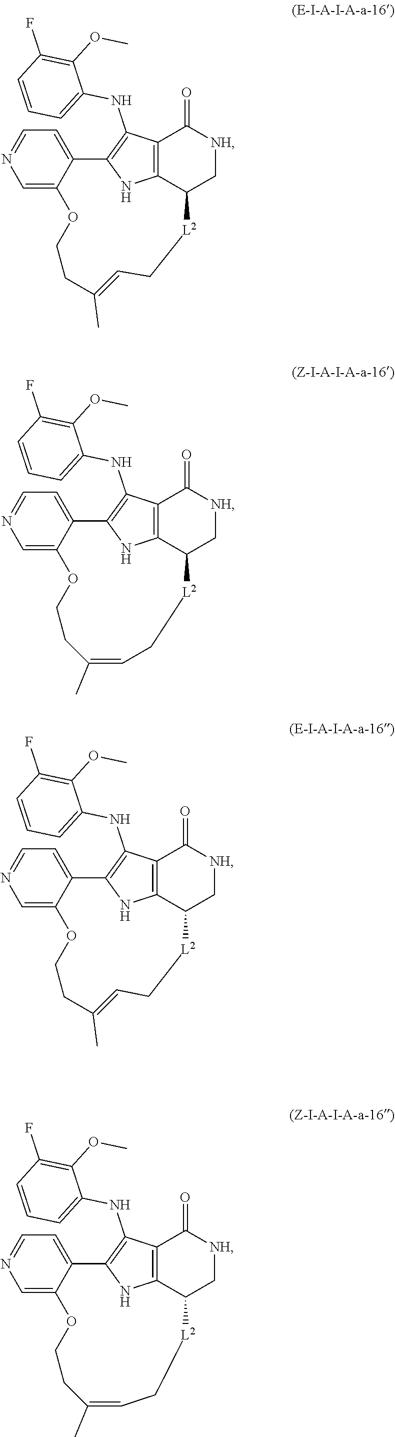

or a pharmaceutically acceptable salt, stereoisomer, solvate, prodrug, isotopic derivative, or tautomer thereof.

In some embodiments, the compound is of Formula (I-A-I-A-a-17):

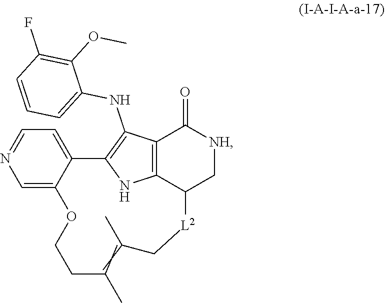

or a pharmaceutically acceptable salt, stereoisomer, solvate, prodrug, isotopic derivative, or tautomer thereof.

In some embodiments, the compound is of Formula (E-I-A-I-A-a-17) or (E-I-A-I-A-a-17):

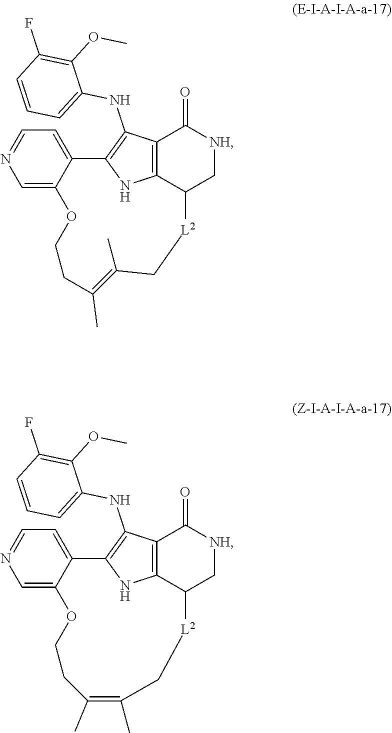

or a pharmaceutically acceptable salt, stereoisomer, solvate, prodrug, isotopic derivative, or tautomer thereof.

In some embodiments, the compound is of Formula (E-I-A-I-A-a-17′), (E-I-A-I-A-a-17″), (Z-I-A-I-A-a-17′), or (Z-I-A-I-A-a-17″):

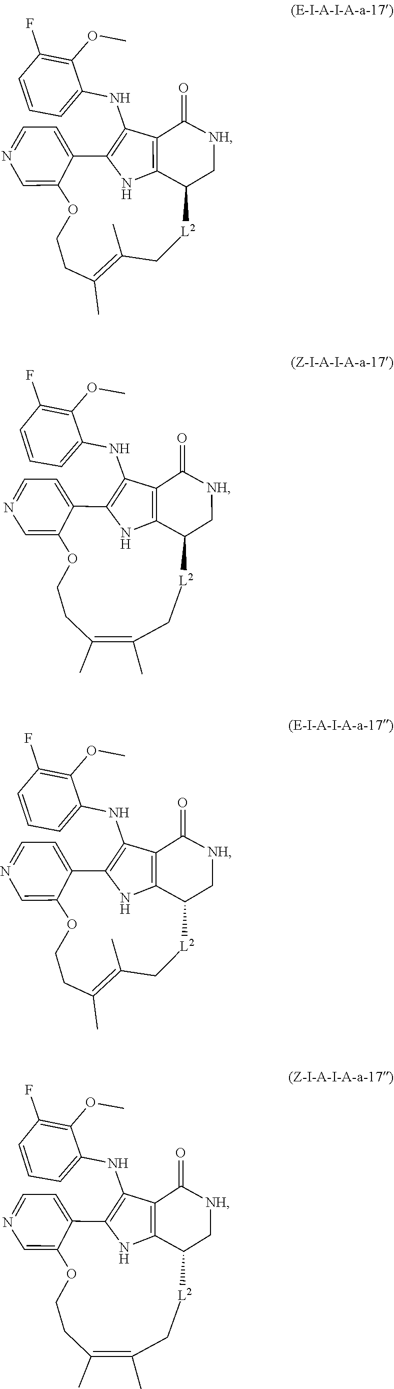

or a pharmaceutically acceptable salt, stereoisomer, solvate, pro rug, isotopic derivative, or tautomer thereof.

In some embodiments, the compound is of Formula (I-A-I-A-a-18):

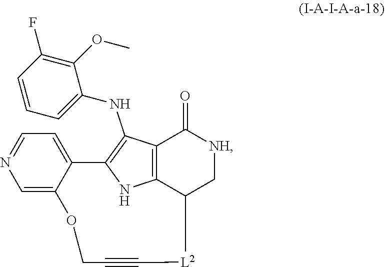

or a pharmaceutically acceptable salt, stereoisomer, solvate, prodrug, isotopic derivative, or tautomer thereof.

In some embodiments, the compound is of Formula (I-A-I-A-a-18′) or (I-A-I-A-a-18″):

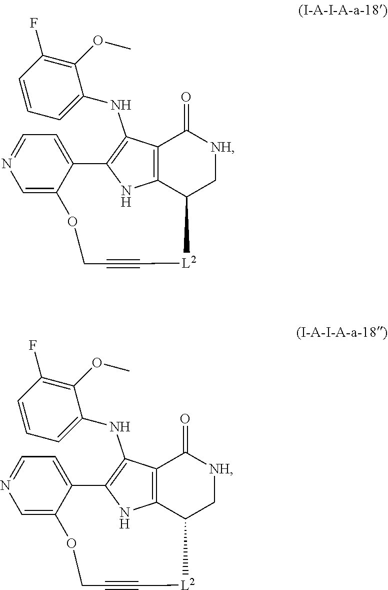

or a pharmaceutically acceptable salt, stereoisomer, solvate, prodrug, isotopic derivative, or tautomer thereof.

In some embodiments, the compound is of Formula (I-A-I-A-a-19):

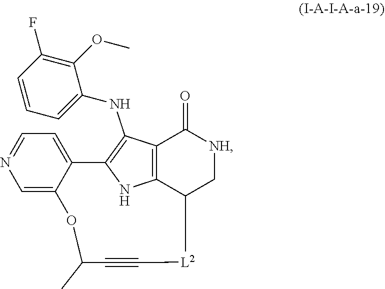

or a pharmaceutically acceptable salt, stereoisomer, solvate, prodrug, isotopic derivative, or tautomer thereof.

In some embodiments, the compound is of Formula (I-A-I-A-a-19′) or (I-A-I-A-a-19″):

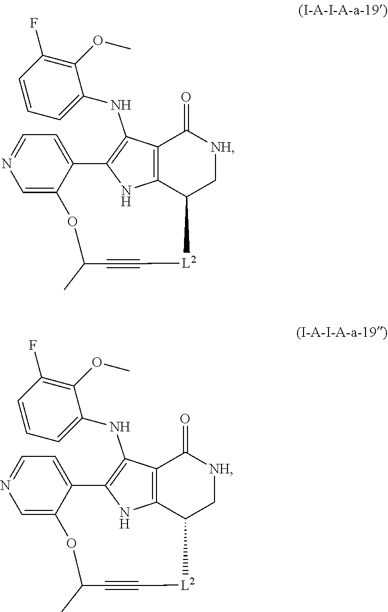

or a pharmaceutically acceptable salt, stereoisomer, solvate, prodrug, isotopic derivative, or tautomer thereof.

In some embodiments, the compound is of Formula (I-A-I-A-a-20):

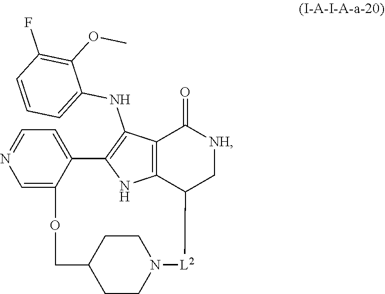

or a pharmaceutically acceptable salt, stereoisomer, solvate, prodrug, isotopic derivative, or tautomer thereof.

In some embodiments, the compound is of Formula (I-A-I-A-a-20′) or (I-A-I-A-a-20″):

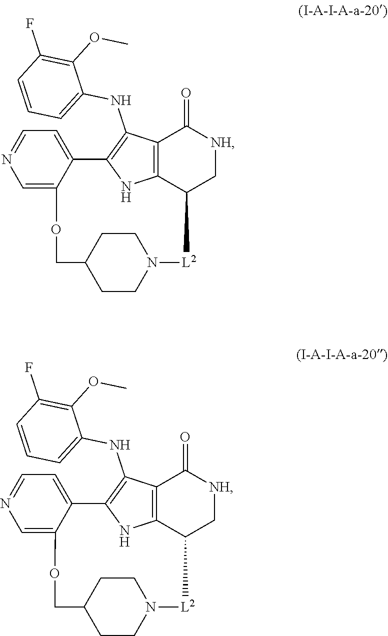

or a pharmaceutically acceptable salt, stereoisomer, solvate, prodrug, isotopic derivative, or tautomer thereof.

In some embodiments, the compound is of Formula (I-A-I-A-a-21):

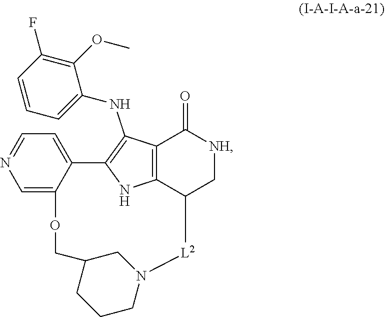

or a pharmaceutically acceptable salt, stereoisomer, solvate, prodrug, isotopic derivative, or tautomer thereof.

In some embodiments, the compound is of Formula (I-A-I-A-a-21′) or (I-A-I-A-a-21″):

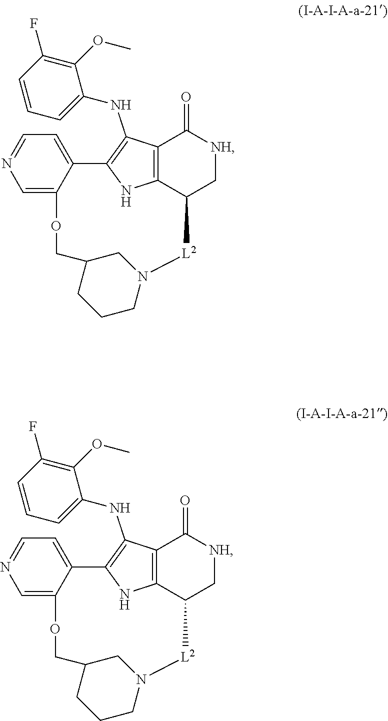

or a pharmaceutically acceptable salt, stereoisomer, solvate, prodrug, isotopic derivative, or tautomer thereof.

In some embodiments, the compound is of Formula (I-A-I-A-a-22):

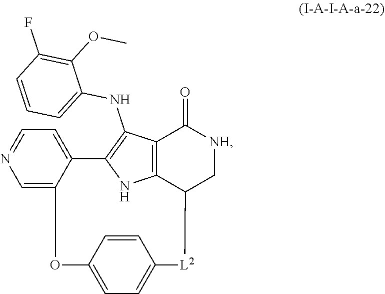

or a pharmaceutically acceptable salt, stereoisomer, solvate, prodrug, isotopic derivative, or tautomer thereof.

In some embodiments, the compound is of Formula (I-A-I-A-a-22′) or (I-A-I-A-a-22″)

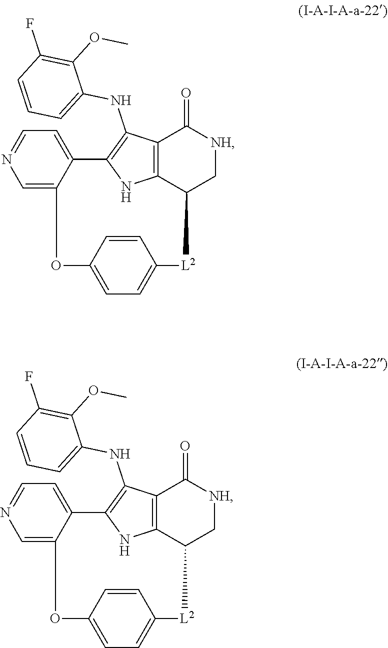

or a pharmaceutically acceptable salt, stereoisomer, solvate, prodrug, isotopic derivative, or tautomer thereof.

In some embodiments, the compound is of Formula (I-A-I-A-a-23):

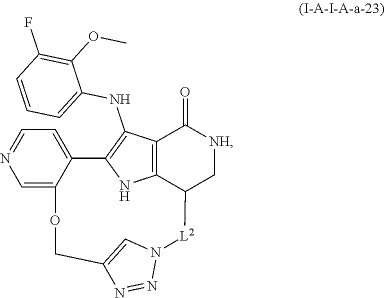

or a pharmaceutically acceptable salt, stereoisomer, solvate, prodrug, isotopic derivative, or tautomer thereof.

In some embodiments, the compound is of Formula (I-A-I-A-a-23′) or (I-A-I-A-a-23″):

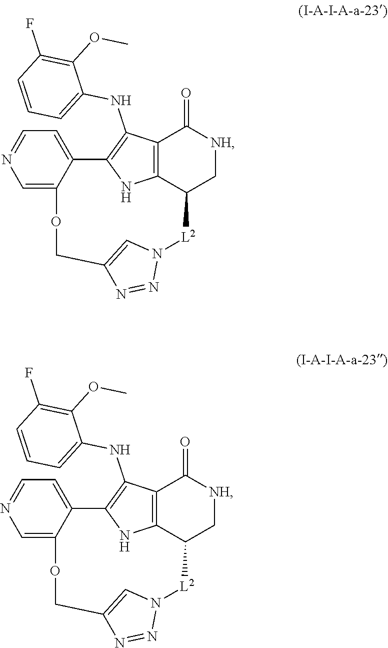

or a pharmaceutically acceptable salt, stereoisomer, solvate, prodrug, isotopic derivative, or tautomer thereof.

In some embodiments, the compound is of Formula (I-A-I-A-a-24):

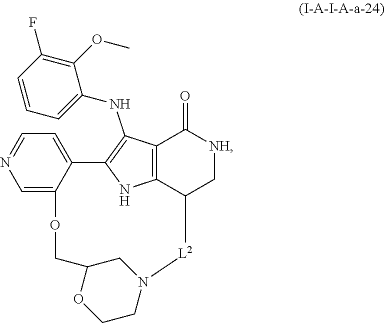

or a pharmaceutically acceptable salt, stereoisomer, solvate, prodrug, isotopic derivative, or tautomer thereof.

In some embodiments, the compound is of Formula (I-A-I-A-a-24′), (I-A-I-A-a-24″), (I-A-I-A-a-24″′), or (I-A-I-A-a-24″″):

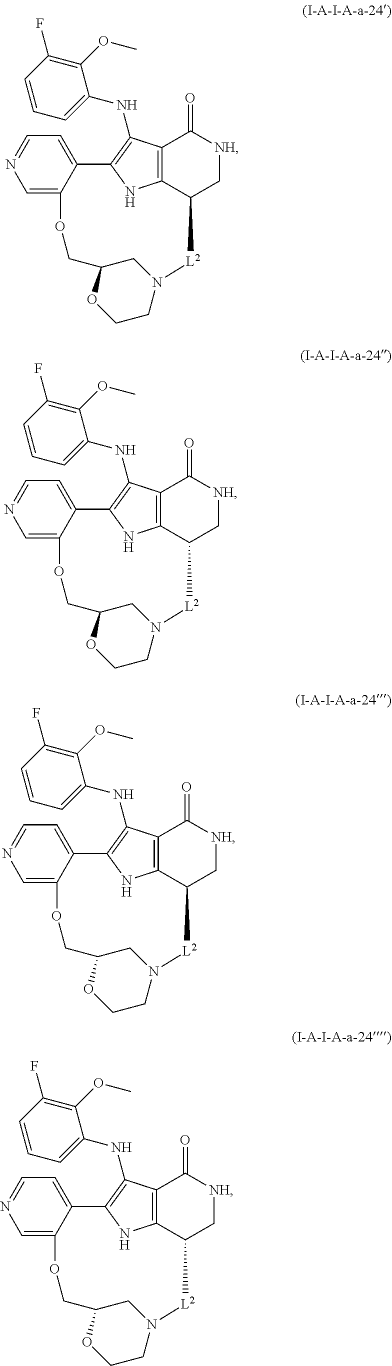

or a pharmaceutically acceptable salt, stereoisomer, solvate, prodrug, isotopic derivative, or tautomer thereof.

In some embodiments, the compound is of Formula (I-A-I-A-a-25):

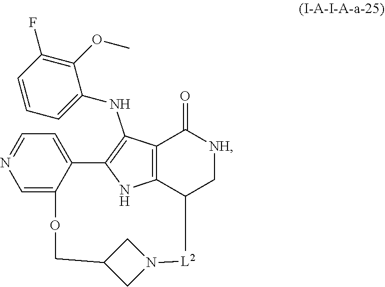

or a pharmaceutically acceptable salt, stereoisomer, solvate, prodrug, isotopic derivative, or tautomer thereof.

In some embodiments, the compound is of Formula (I-A-I-A-a-25′), (I-A-I-A-a-25″), (I-A-I-A-a-25″′), or (I-A-I-A-a-25″″):

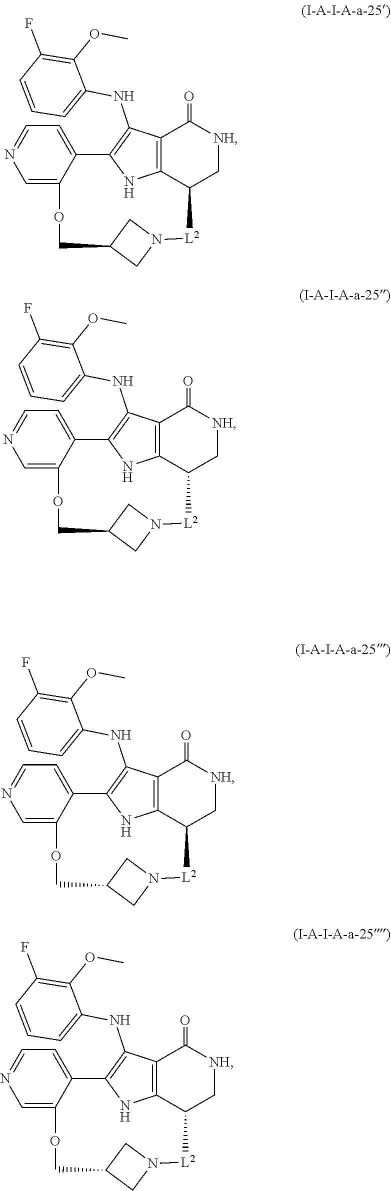

or a pharmaceutically acceptable salt, stereoisomer, solvate, prodrug, isotopic derivative, or tautomer thereof.

In some embodiments, the compound is ofFormula (I-A-I-A-a-26):

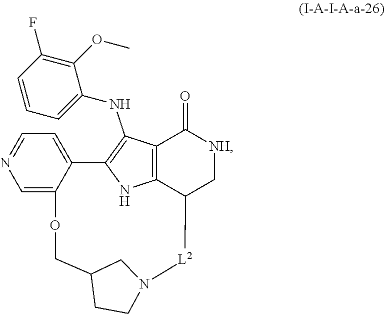

or a pharmaceutically acceptable salt, stereoisomer, solvate, prodrug, isotopic derivative, or tautomer thereof.

In some embodiments, the compound is of Formula (I-A-I-A-a-26′), (I-A-I-A-a-26″), (I-A-I-A-a-26″′), or (I-A-I-A-a-26″″):

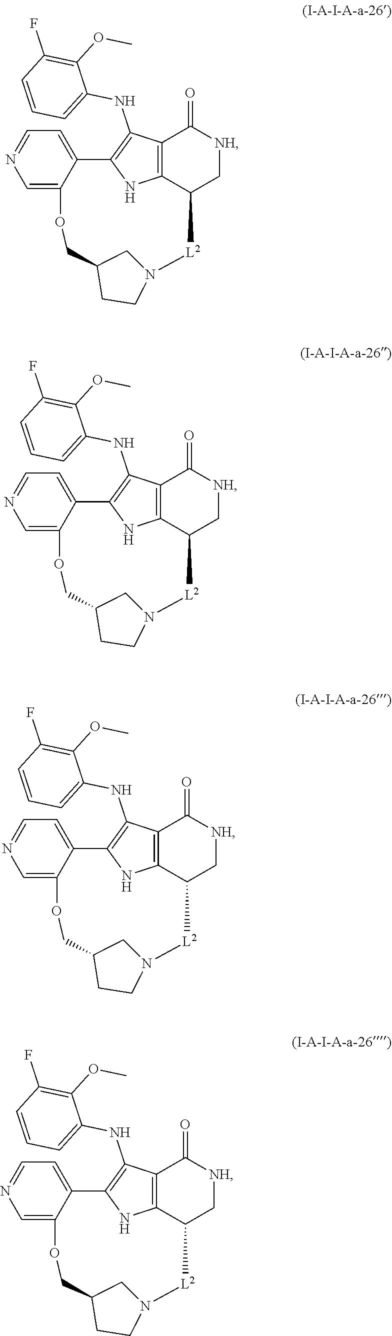

or a pharmaceutically acceptable salt, stereoisomer, solvate, prodrug, isotopic derivative, or tautomer thereof.

In some embodiments, the compound is of Formula (I-A-I-A-a-27):

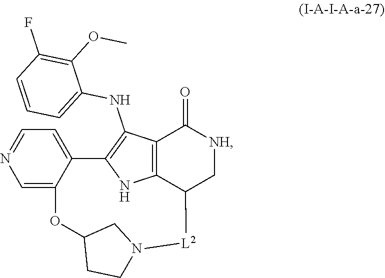

or a pharmaceutically acceptable salt, stereoisomer, solvate, prodrug, isotopic derivative, or tautomer thereof.

In some embodiments, the compound is of Formula (I-A-I-A-a-27′), (I-A-I-A-a-27″), (I-A-I-A-a-27′″), or (I-A-I-A-a-27″″):

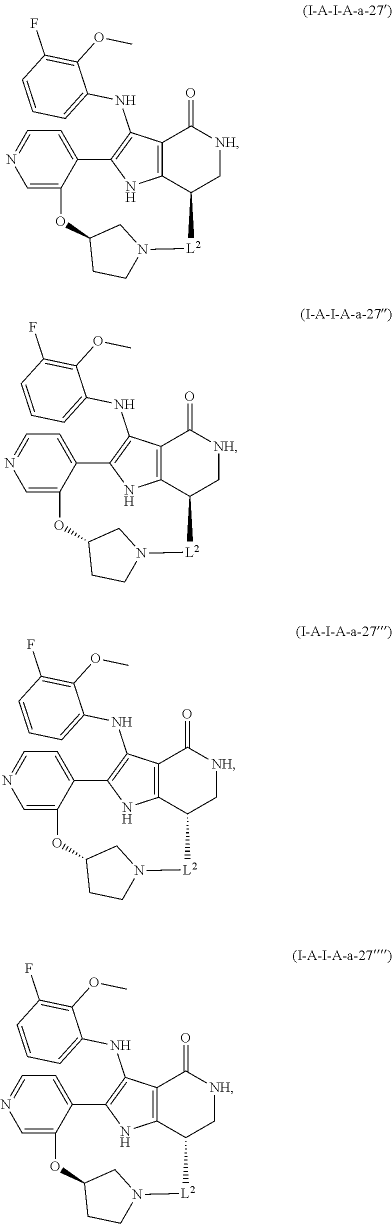

or a pharmaceutically acceptable salt, stereoisomer, solvate, prodrug, isotopic derivative, or tautomer thereof.

In some embodiments, the compound is of Formula (I-A-I-A-a-28):

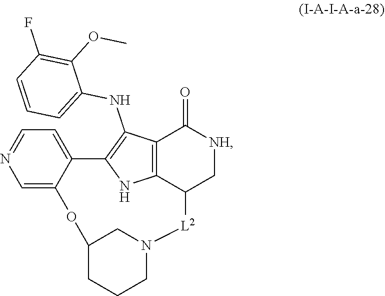

or a pharmaceutically acceptable salt, stereoisomer, solvate, prodrug, isotopic derivative, or tautomer thereof.

In some embodiments, the compound is of Formula (I-A-I-A-a-28′), (I-A-I-A-a-28″), (I-A-I-A-a-28″′), or (I-A-I-A-a-28″″):

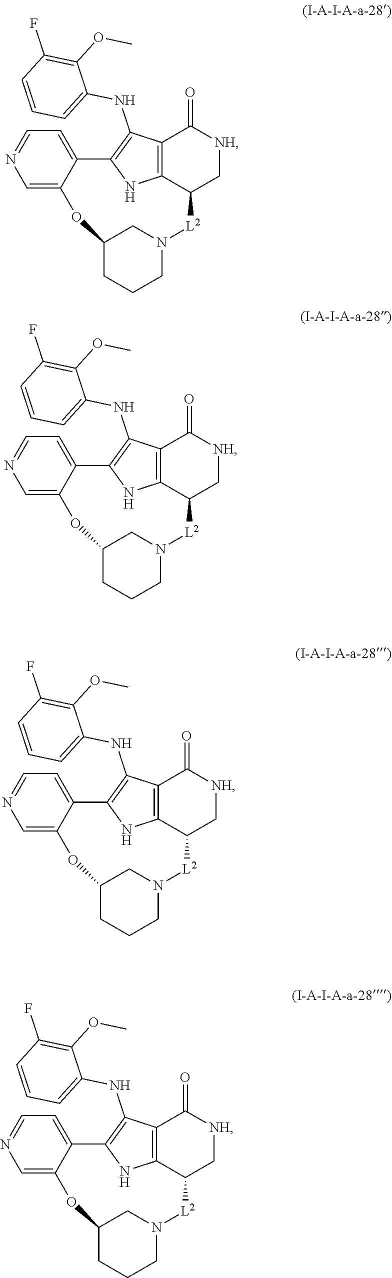

or a pharmaceutically acceptable salt, stereoisomer, solvate, prodrug, isotopic derivative, or tautomer thereof.

In some embodiments, the compound is of Formula (I-A-I-A-a-29):

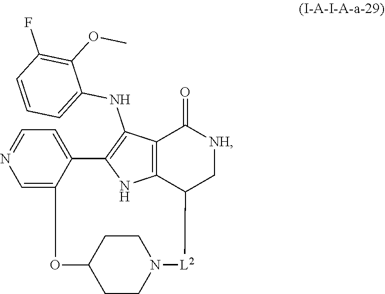

or a pharmaceutically acceptable salt, stereoisomer, solvate, prodrug, isotopic derivative, or tautomer thereof.

In some embodiments, the compound is of Formula (I-A-I-A-a-29′), (I-A-I-A-a-29″), (I-A-I-A-a-29″′), or (I-A-I-A-a-29″″):

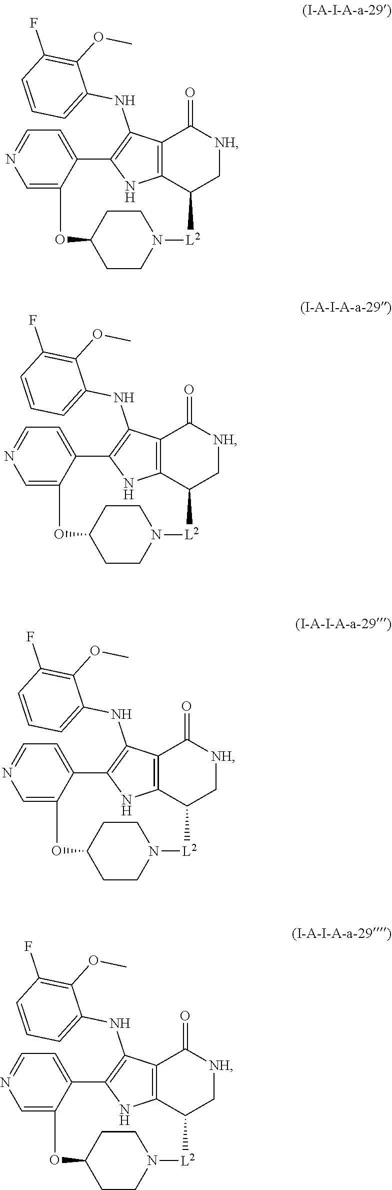

or a pharmaceutically acceptable salt, stereoisomer, solvate, prodrug, isotopic derivative, or tautomer thereof.

In some embodiments, the compound is of Formula (I-A-I-A-a-30):

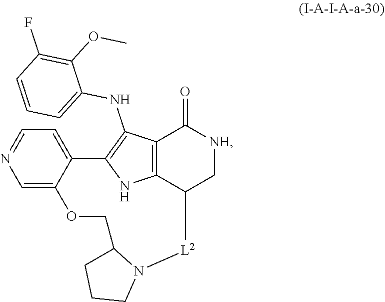

or a pharmaceutically acceptable salt, stereoisomer, solvate, prodrug, isotopic derivative, or tautomer thereof.

In some embodiments, the compound is of Formula (I-A-I-A-a-30′), (I-A-I-A-a-30″), (I-A-I-A-a-30″′), or (I-A-I-A-a-30″″):

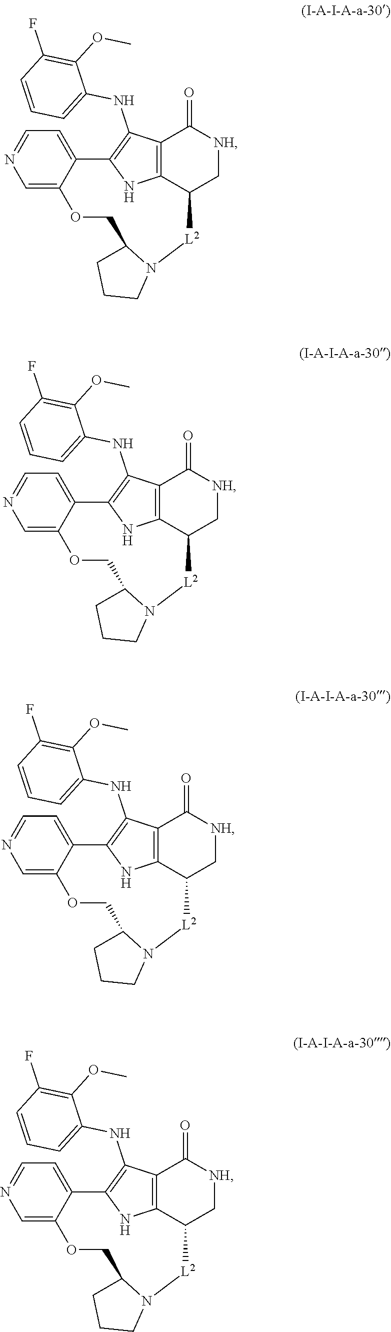

or a pharmaceutically acceptable salt, stereoisomer, solvate, prodrug, isotopic derivative, or tautomer thereof.

In some embodiments, the compound is of Formula (I-A-I-A-b-1):

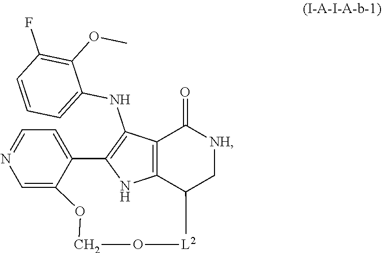

or a pharmaceutically acceptable salt, stereoisomer, solvate, prodrug, isotopic derivative, or tautomer thereof.

In some embodiments, the compound is of Formula (I-A-I-A-b-1′) or (I-A-I-A-b-1″):

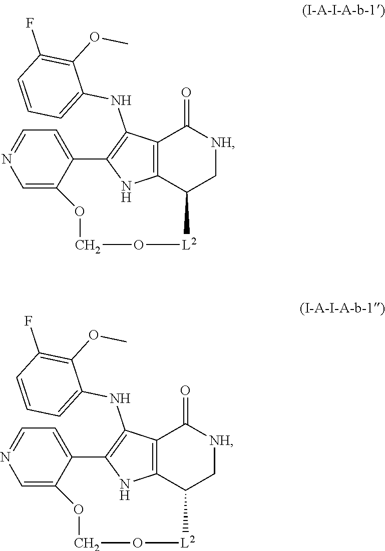

or a pharmaceutically acceptable salt, stereoisomer, solvate, prodrug, isotopic derivative, or tautomer thereof.

In some embodiments, the compound is of Formula (I-A-I-A-b-2):

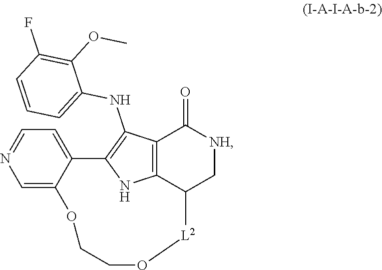

or a pharmaceutically acceptable salt, stereoisomer, solvate, prodrug, isotopic derivative, or tautomer thereof.

In some embodiments, the compound is of Formula (I-A-I-A-b-2′) or (I-A-I-A-b-2″):

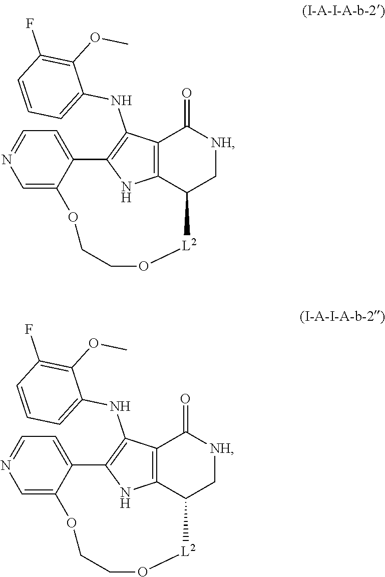

or a pharmaceutically acceptable salt, stereoisomer, solvate, prodrug, isotopic derivative, or tautomer thereof.

In some embodiments, the compound is of Formula (I-A-I-A-b-3):

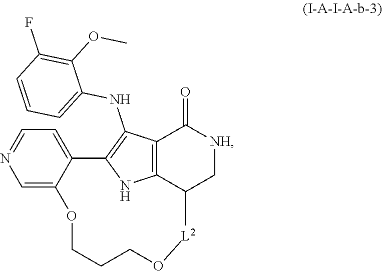

or a pharmaceutically acceptable salt, stereoisomer, solvate, prodrug, isotopic derivative, or tautomer thereof.

In some embodiments, the compound is of Formula (I-A-I-A-a-3′) or (I-A-I-A-a-3″):

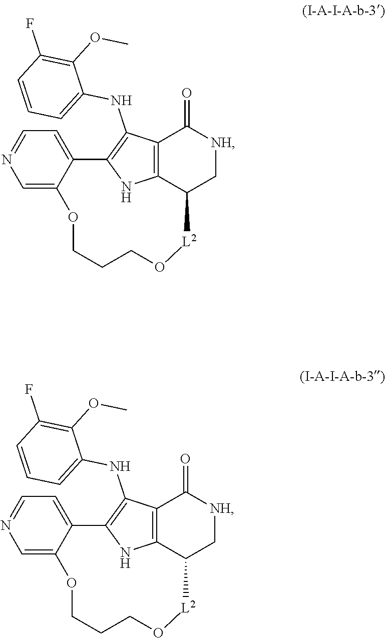

or a pharmaceutically acceptable salt, stereoisomer, solvate, prodrug, isotopic derivative, or tautomer thereof.

In some embodiments, the compound is of Formula (I-A-I-A-b-4):

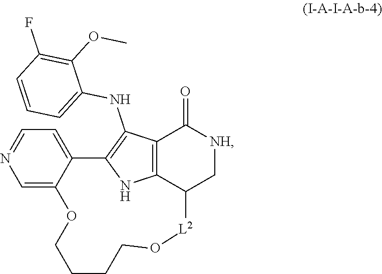

or a pharmaceutically acceptable salt, stereoisomer, solvate, prodrug, isotopic derivative, or tautomer thereof.

In some embodiments, the compound is of Formula (I-A-I-A-b-4′) or (I-A-I-A-b-4″):

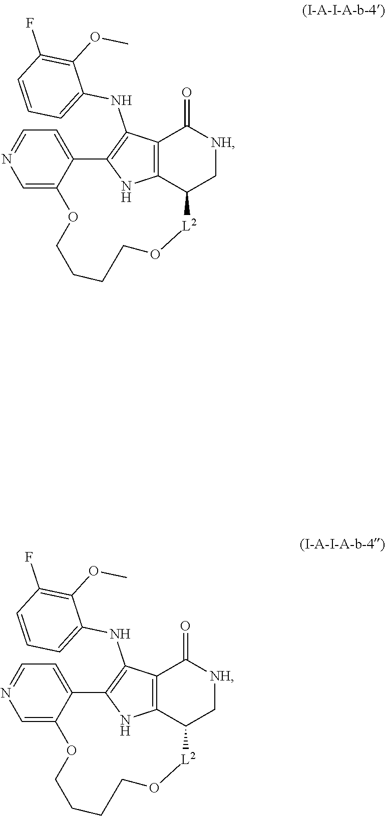

or a pharmaceutically acceptable salt, stereoisomer, solvate, prodrug, isotopic derivative, or tautomer thereof.

In some embodiments, the compound is of Formula (I-A-I-A-b-5):

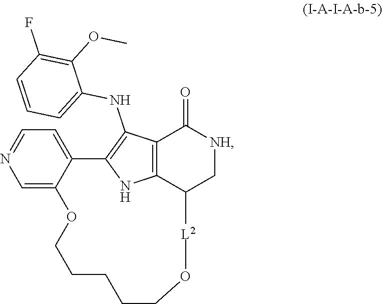

or a pharmaceutically acceptable salt, stereoisomer, solvate, prodrug, isotopic derivative, or tautomer thereof.

In some embodiments, the compound is of Formula (I-A-I-A-b-5′) or (I-A-I-A-b-5″):

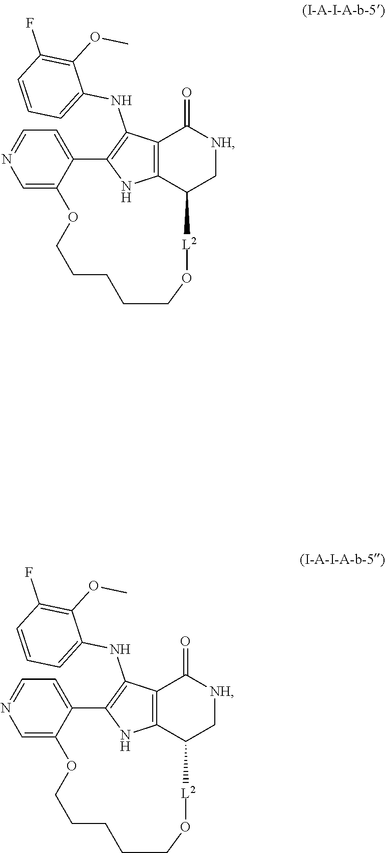

or a pharmaceutically acceptable salt, stereoisomer, solvate, prodrug, isotopic derivative, or tautomer thereof.

In some embodiments, the compound is of Formula (I-A-I-A-b-6):

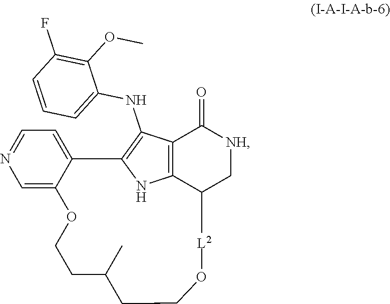

or a pharmaceutically acceptable salt, stereoisomer, solvate, prodrug, isotopic derivative, or tautomer thereof.

In some embodiments, the compound is of Formula (I-A-I-A-a-6′) or (I-A-I-A-a-6″):

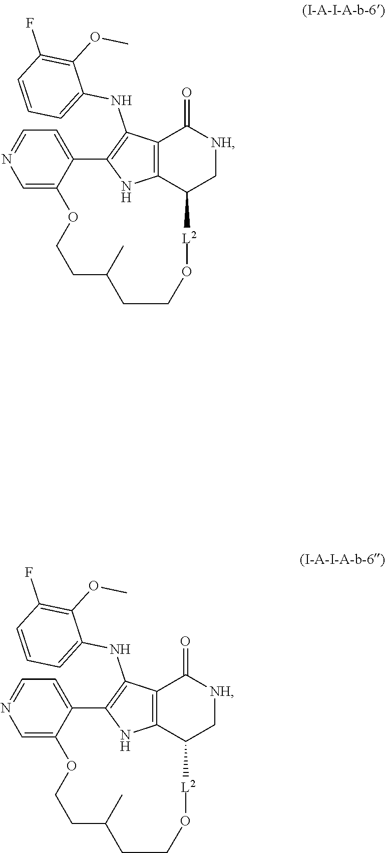

or a pharmaceutically acceptable salt, stereoisomer, solvate, prodrug, isotopic derivative, or tautomer thereof.

In some embodiments, the compound is of Formula (I-A-I-A-b-7):

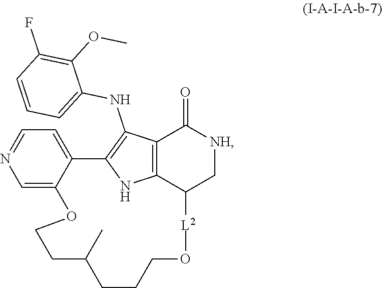

or a pharmaceutically acceptable salt, stereoisomer, solvate, prodrug, isotopic derivative, or tautomer thereof.

In some embodiments, the compound is of Formula (I-A-I-A-b-7′) or (I-A-I-A-b-7″):

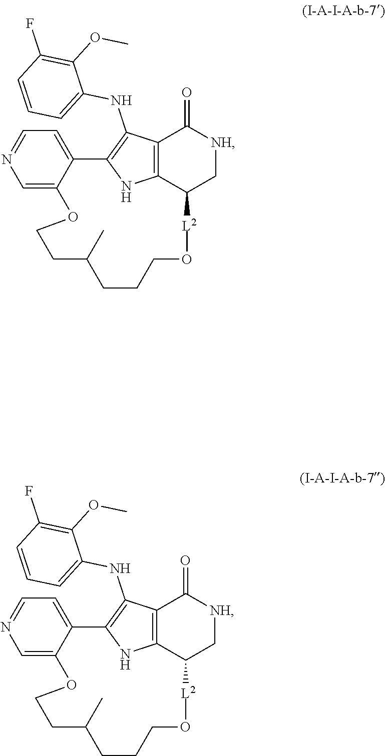

or a pharmaceutically acceptable salt, stereoisomer, solvate, prodrug, isotopic derivative, or tautomer thereof.

In some embodiments, the compound is of Formula (I-A-I-A-b-8):

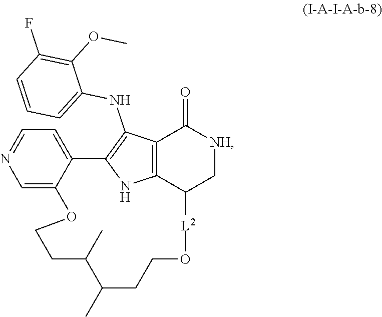

or a pharmaceutically acceptable salt, stereoisomer, solvate, prodrug, isotopic derivative, or tautomer thereof.

In some embodiments, the compound is of Formula (I-A-I-A-b-8′) or (I-A-I-A-b-8″):

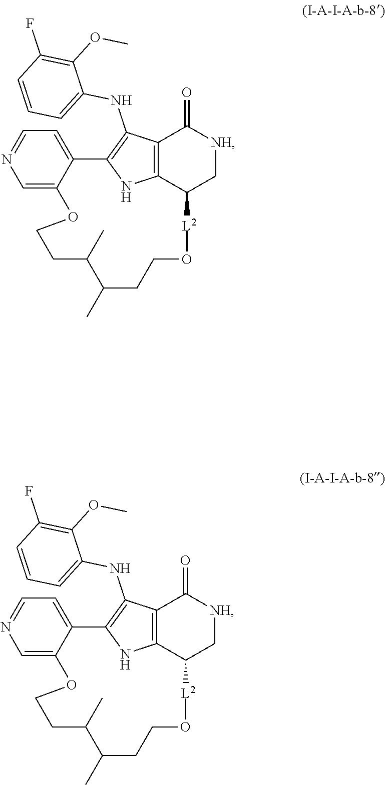

or a pharmaceutically acceptable salt, stereoisomer, solvate, prodrug, isotopic derivative, or tautomer thereof.

In some embodiments, the compound is of Formula (I-A-I-A-b-9):

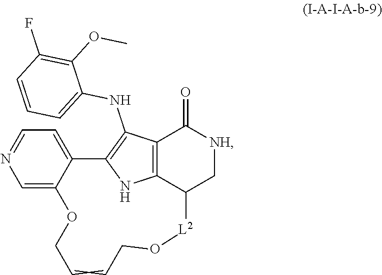

or a pharmaceutically acceptable salt, stereoisomer, solvate, prodrug, isotopic derivative, or tautomer thereof.

In some embodiments, the compound is of Formula (E-I-A-I-A-b-9) or (Z-I-A-I-A-b-9):

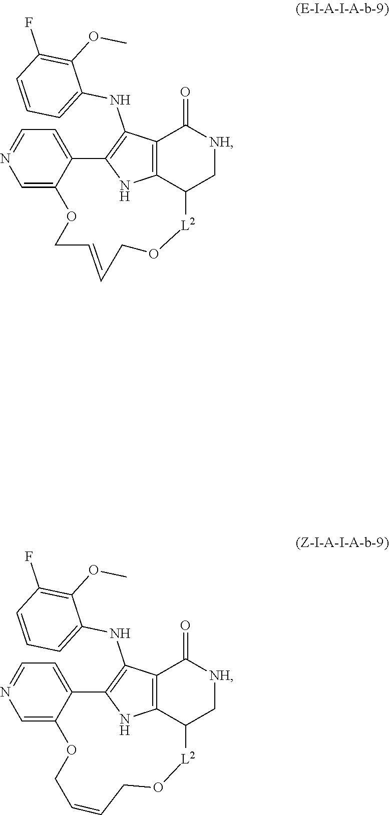

or a pharmaceutically acceptable salt, stereoisomer, solvate, prodrug, isotopic derivative, or tautomer thereof.

In some embodiments, the compound is of Formula (E-I-A-I-A-b-9′), (E-I-A-I-A-b-9″), (Z-I-A-I-A-b-9′), (Z-I-A-I-A-b-9″):

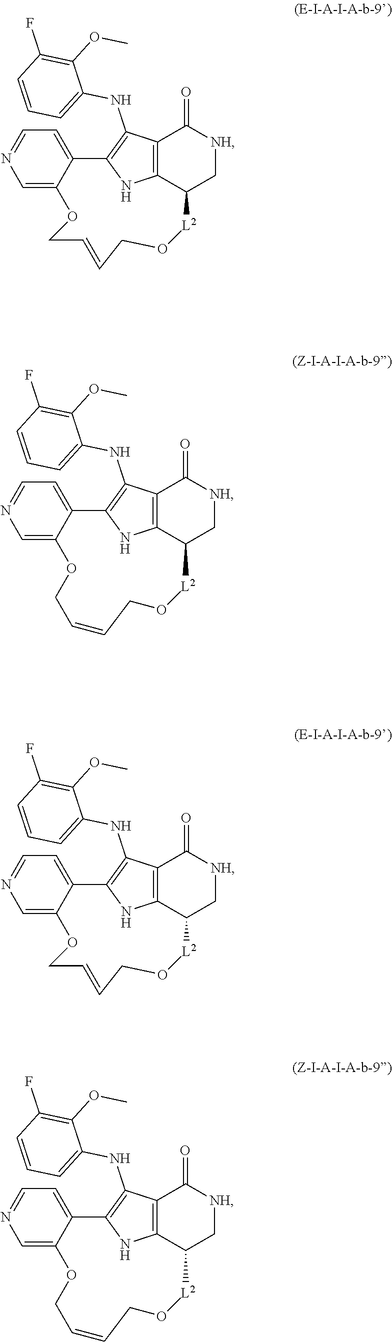

or a pharmaceutically acceptable salt, stereoisomer, solvate, prodrug, isotopic derivative, or tautomer thereof.

In some embodiments, the compound is of Formula (I-A-I-A-b-10):

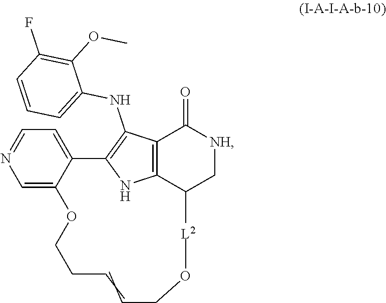

or a pharmaceutically acceptable salt, stereoisomer, solvate, prodrug, isotopic derivative, or tautomer thereof.

In some embodiments, the compound is of Formula (E-I-A-I-A-b-10) or (Z-I-A-I-A-b-10):

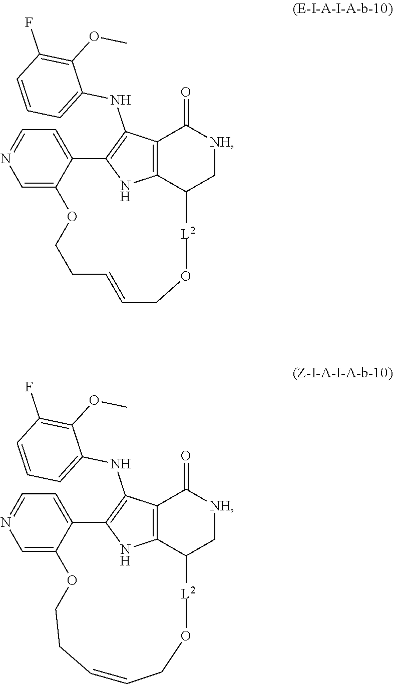

or a pharmaceutically acceptable salt, stereoisomer, solvate, prodrug, isotopic derivative, or tautomer thereof.

In some embodiments, the compound is of Formula (E-I-A-I-A-b-10′), (E-I-A-I-A-b-10″), (Z-I-A-I-A-b-10′), or (Z-I-A-1-A-b-10″):

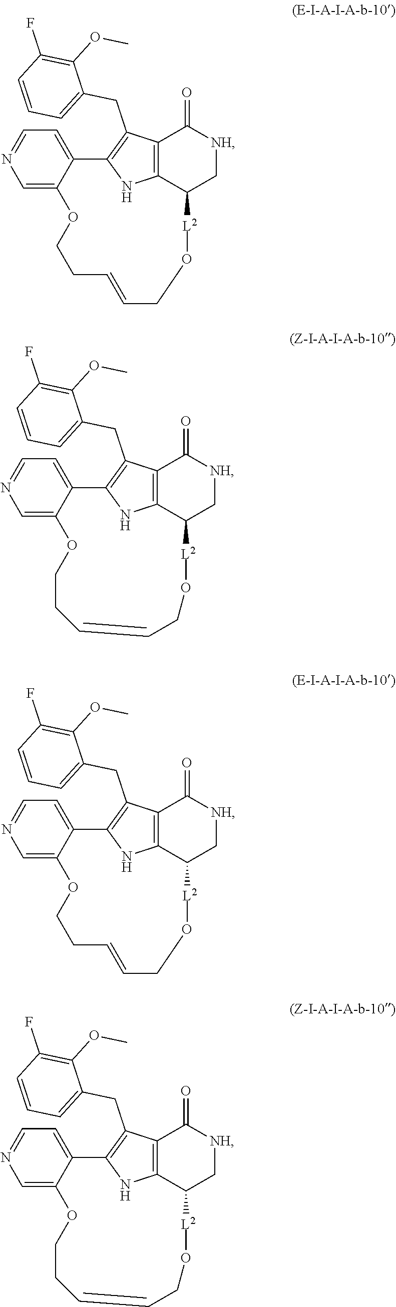

or a pharmaceutically acceptable salt, stereoisomer, solvate, prodrug, isotopic derivative, or tautomer thereof.

In some embodiments, the compound is of Formula (I-A-I-A-b-11):

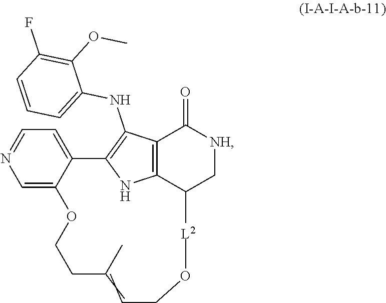

or a pharmaceutically acceptable salt, stereoisomer, solvate, prodrug, isotopic derivative, or tautomer thereof.

In some embodiments, the compound is of Formula (E-I-A-I-A-b-11) or (Z-I-A-I-A-b-11):

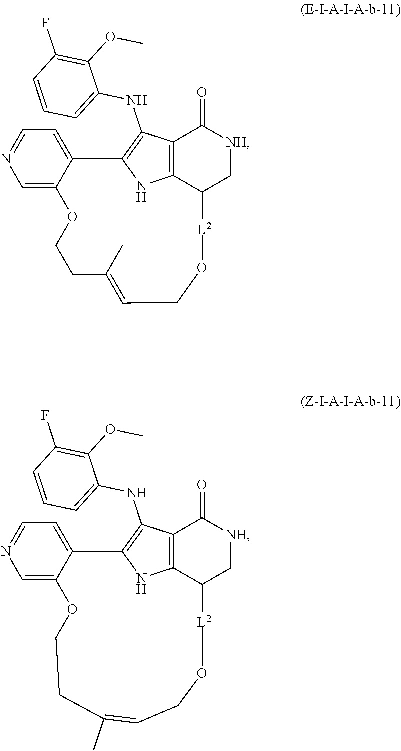

or a pharmaceutically acceptable salt, stereoisomer, solvate, prodrug, isotopic derivative, or tautomer thereof.

In some embodiments, the compound is of Formula (I-A-I-A-b-11′), (I-A-I-A-b-11″), (I-A-I-A-b-11′), or (I-A-I-A-b-11″)

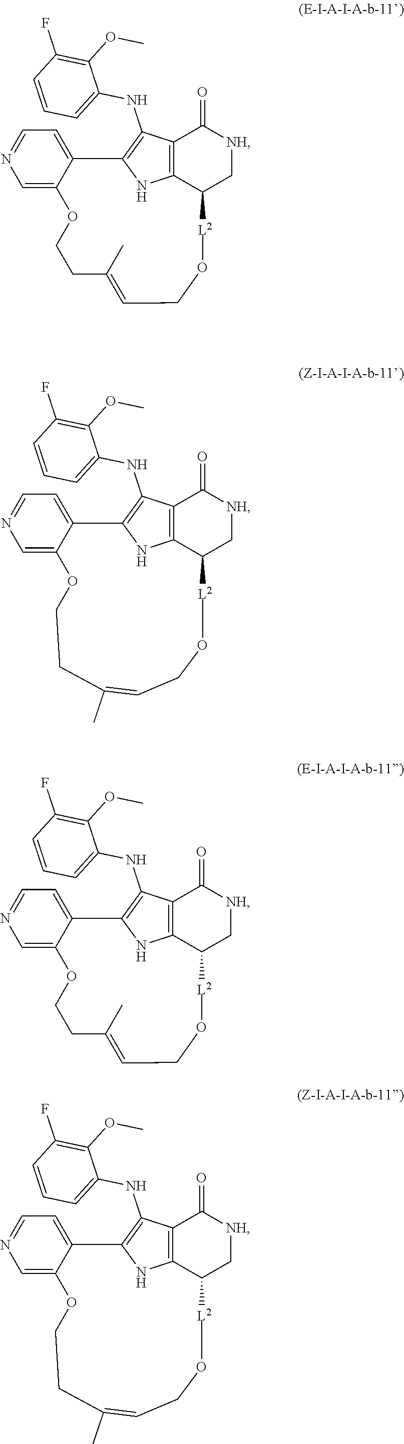

or a pharmaceutically acceptable salt, stereoisomer, solvate, prodrug, isotopic derivative, or tautomer thereof.

In some embodiments, the compound is of Formula (I-A-I-A-b-12):

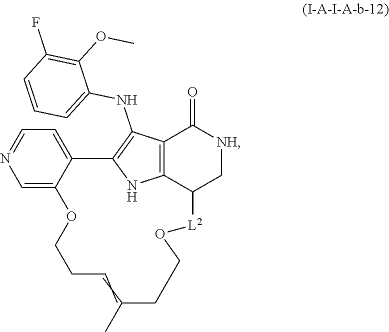

or a pharmaceutically acceptable salt, stereoisomer, solvate, prodrug, isotopic derivative, or tautomer thereof.

In some embodiments, the compound is of Formula (E-I-A-I-A-b-12) or (Z-I-A-I-A-b-12):

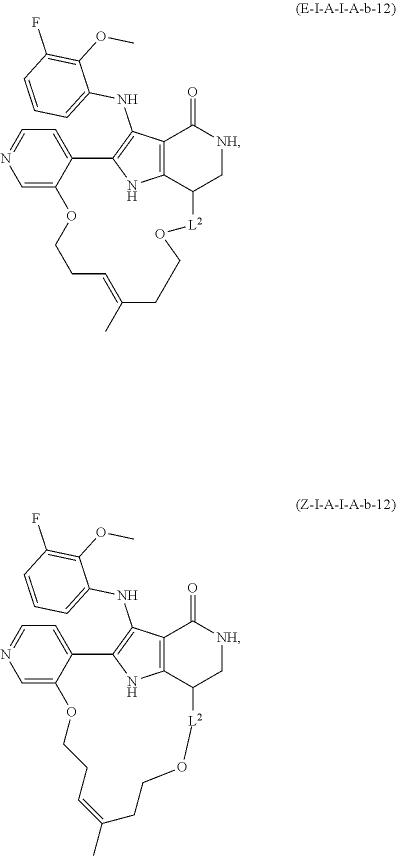

or a pharmaceutically acceptable salt, stereoisomer, solvate, prodrug, isotopic derivative, or tautomer thereof.

In some embodiments, the compound is of Formula (E-I-A-I-A-b-12′), (E-I-A-I-A-b-12″), (Z-I-A-I-A-b-12′), (Z-I-A-I-A-b-12″):

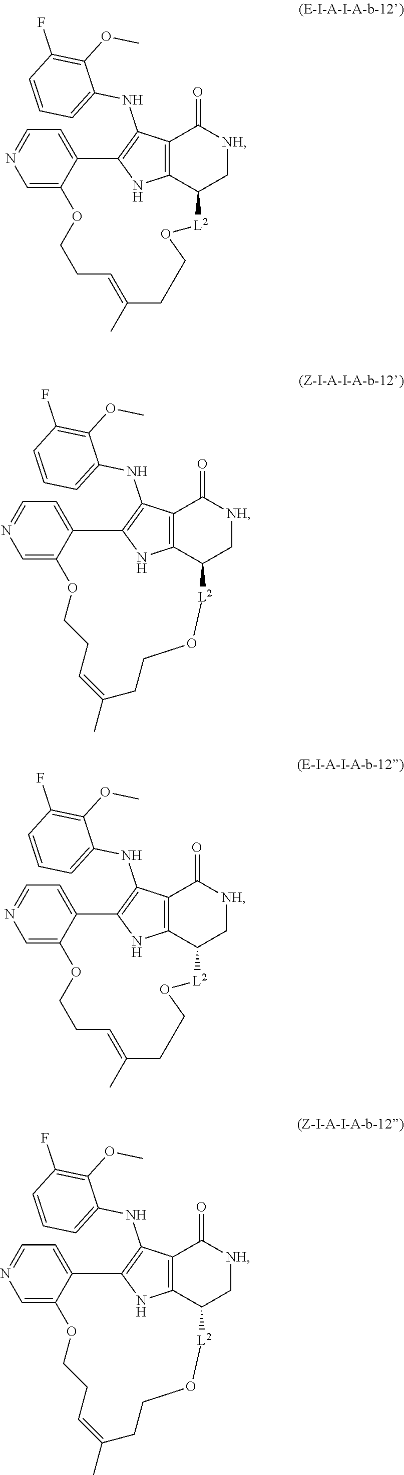

or a pharmaceutically acceptable salt, stereoisomer, solvate, prodrug, isotopic derivative, or tautomer thereof.

In some embodiments, the compound is of Formula (I-A-I-A-b-13):

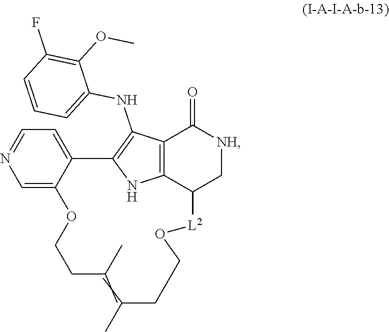

or a pharmaceutically acceptable salt, stereoisomer, solvate, prodrug, isotopic derivative, or tautomer thereof.

In some embodiments, the compound is of Formula (E-I-A-I-A-b-13) or (Z-I-A-I-A-b-13):

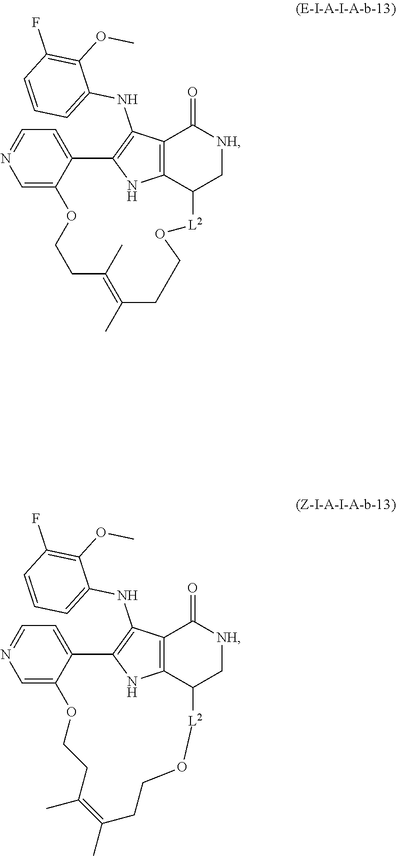

or a pharmaceutically acceptable salt, stereoisomer, solvate, prodrug, isotopic derivative, or tautomer thereof.

In some embodiments, the compound is of Formula (E-I-A-I-A-b-13′), (E-I-A-I-A-b-13″), (Z-I-A-I-A-b-13′), (Z-I-A-I-A-b-13″):

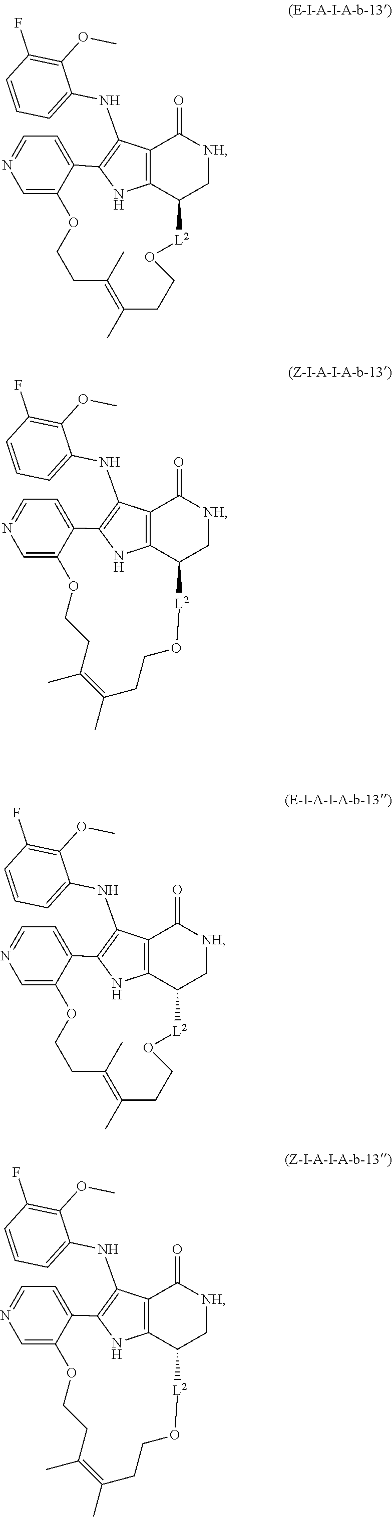

or a pharmaceutically acceptable salt, stereoisomer, solvate, prodrug, isotopic derivative, or tautomer thereof.

In some embodiments, the compound is of Formula (I-A-I-A-b-14):

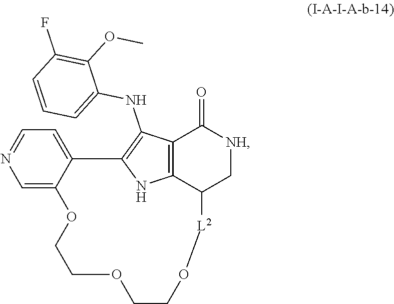

or a pharmaceutically acceptable salt, stereoisomer, solvate, prodrug, isotopic derivative, or tautomer thereof.

In some embodiments, the compound is of Formula (I-A-I-A-b-14′) or (I-A-I-A-b-14″):

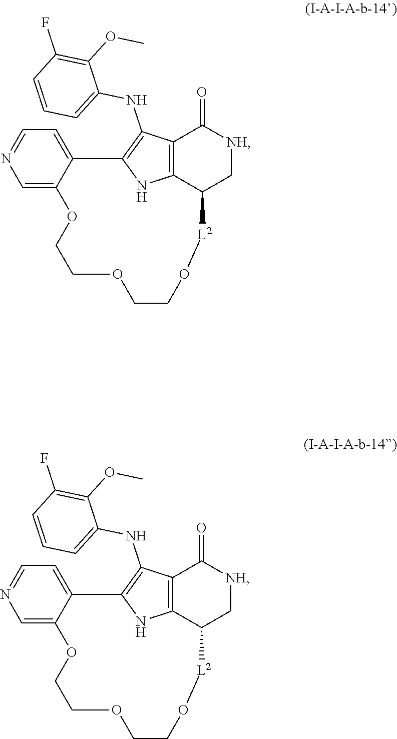

or a pharmaceutically acceptable salt, stereoisomer, solvate, prodrug, isotopic derivative, or tautomer thereof.

In some embodiments, the compound is of Formula (I-A-I-A-b-15):

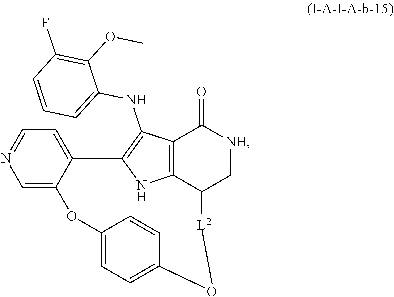

or a pharmaceutically acceptable salt, stereoisomer, solvate, prodrug, isotopic derivative, or tautomer thereof.

In some embodiments, the compound is of Formula (I-A-I-A-b-15′) or (I-A-I-A-b-15″):

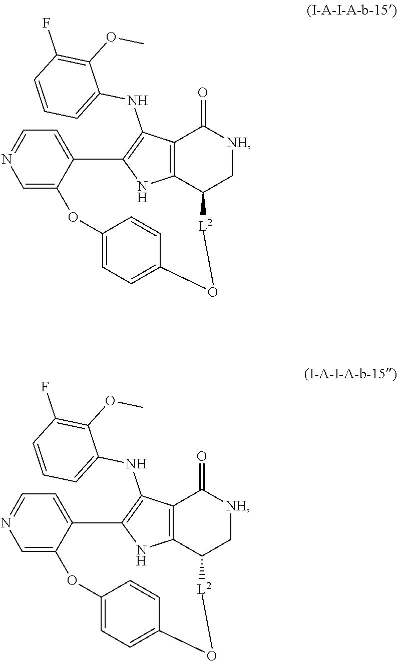

or a pharmaceutically acceptable salt, stereoisomer, solvate, prodrug, isotopic derivative, or tautomer thereof.

In some embodiments, the compound is of Formula (I-A-I-A-b-16):

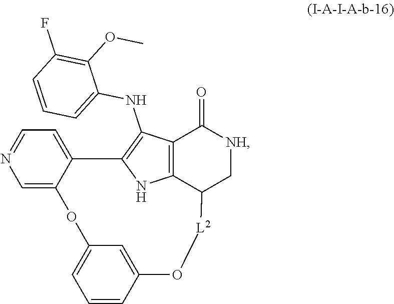

or a pharmaceutically acceptable salt, stereoisomer, solvate, prodrug, isotopic derivative, or tautomer thereof.

In some embodiments, the compound is of Formula (I-A-I-A-b-16′) or (I-A-I-A-b-16″):

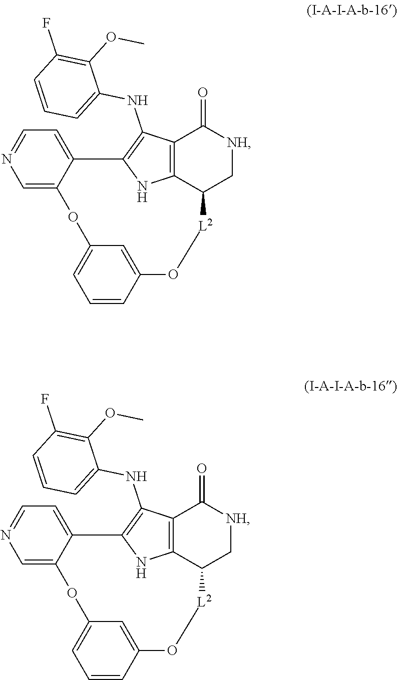

or a pharmaceutically acceptable salt, stereoisomer, solvate, prodrug, isotopic derivative, or tautomer thereof.

In some embodiments, the compound is of Formula (I-A-I-A-b-17):

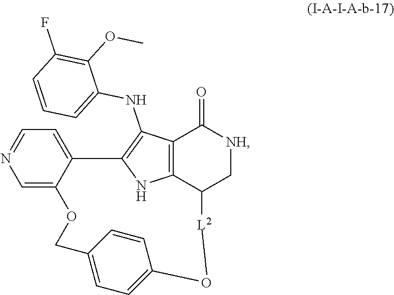

or a pharmaceutically acceptable salt, stereoisomer, solvate, prodrug, isotopic derivative, or tautomer thereof.

In some embodiments, the compound is of Formula (I-A-I-A-b-17′) or (I-A-I-A-b-17″):

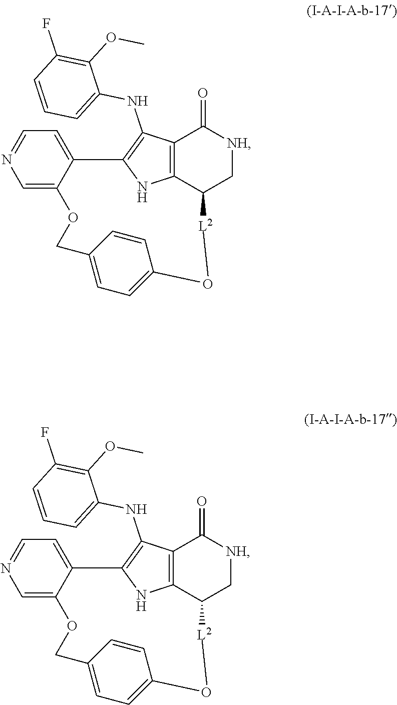

or a pharmaceutically acceptable salt, stereoisomer, solvate, prodrug, isotopic derivative, or tautomer thereof.

In some embodiments, the compound is of Formula (I-A-I-A-b-18):

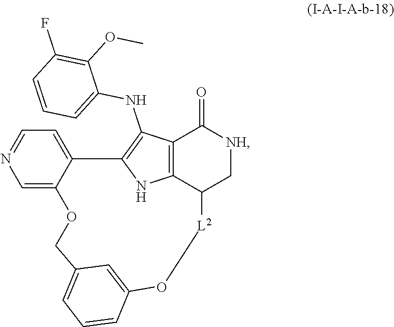

or a pharmaceutically acceptable salt, stereoisomer, solvate, prodrug, isotopic derivative, or tautomer thereof.

In some embodiments, the compound is of Formula (I-A-I-A-b-18′) or (I-A-I-A-b-18″):

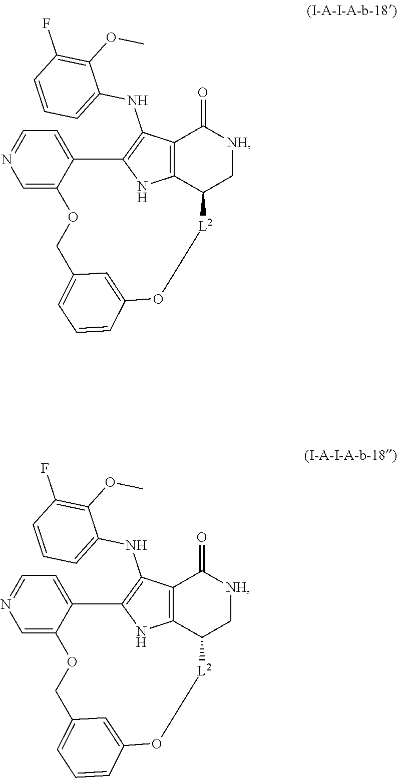

or a pharmaceutically acceptable salt, stereoisomer, solvate, prodrug, isotopic derivative, or tautomer thereof.

In some embodiments, the compound is of Formula (I-A-I-A-b-19):

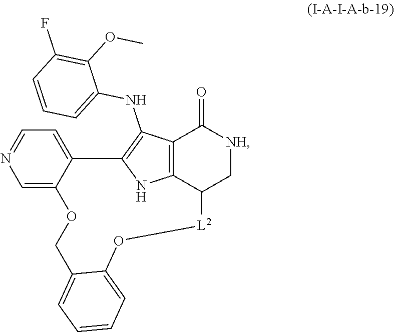

or a pharmaceutically acceptable salt, stereoisomer, solvate, prodrug, isotopic derivative, or tautomer thereof.

In some embodiments, the compound is of Formula (I-A-I-A-b-19′) or (I-A-I-A-b-19″):

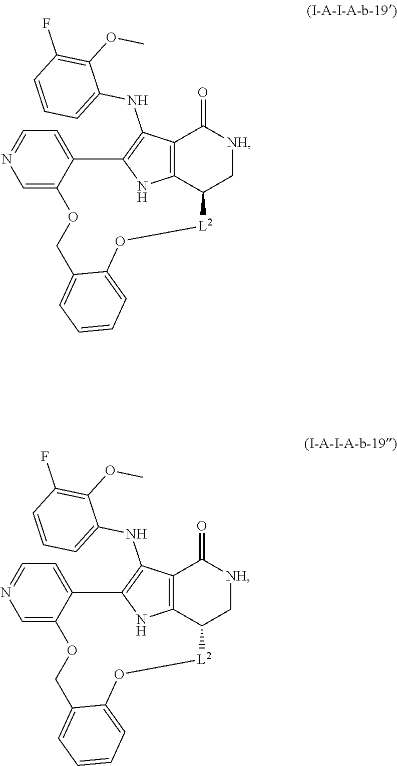

or a pharmaceutically acceptable salt, stereoisomer, solvate, prodrug, isotopic derivative, or tautomer thereof.

In some embodiments, the compound is of Formula (I-A-I A-c-1:

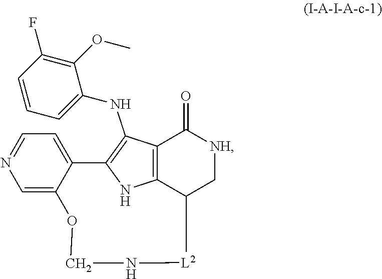

or a pharmaceutically acceptable salt, stereoisomer, solvate, prodrug, isotopic derivative, or tautomer thereof.

In some embodiments, the compound is of Formula (I-A-I-A-c-1′) or (I-A-I-A-c-1″):

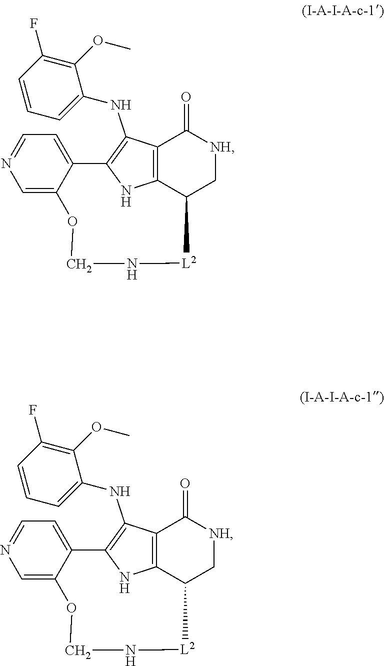

or a pharmaceutically acceptable salt, stereoisomer, solvate, prodrug, isotopic derivative, or tautomer thereof.

In some embodiments, the compound is of Formula (I-A-I-A-c-2):

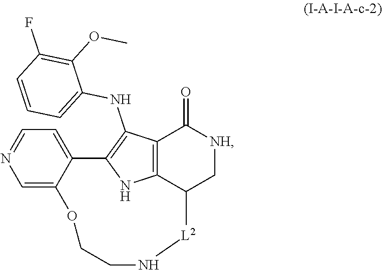

or a pharmaceutically acceptable salt, stereoisomer, solvate, prodrug, isotopic derivative, or tautomer thereof.

Tn some embodiments, the compound is of Formula (T-A-T-A-c-2′) or (T-A-A-c-2

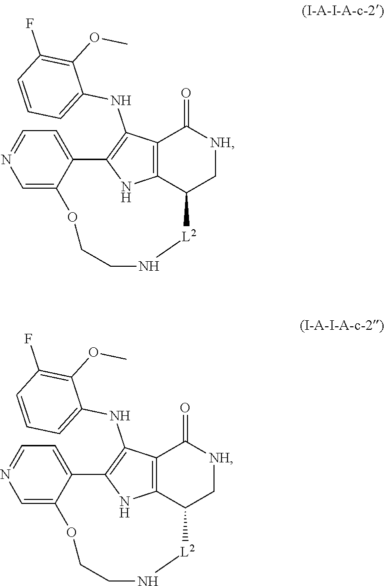

or a pharmaceutically acceptable salt, stereoisomer, solvate, prodrug, isotopic derivative, or tautomer thereof.

In some embodiments, the compound is of Formula (I-A-I-A-c-3):

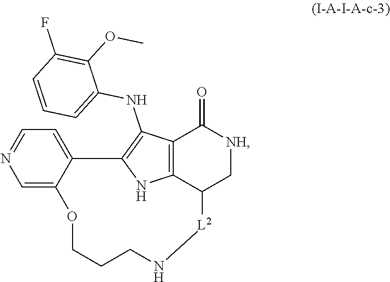

or a pharmaceutically acceptable salt, stereoisomer, solvate, prodrug, isotopic derivative, or tautomer thereof.

In some embodiments, the compound is of Formula (I-A-I-A-c-3′) or (I-A-I-A-c-3″):

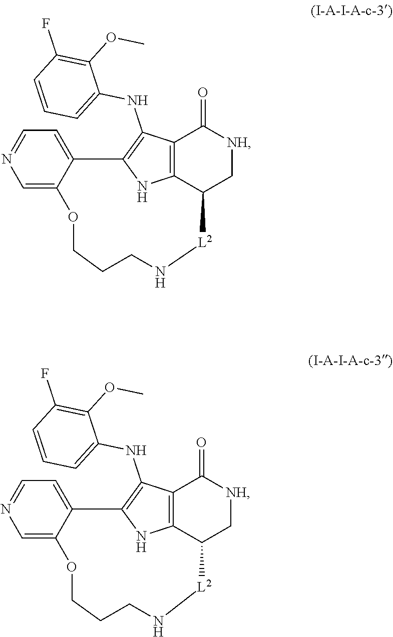

or a pharmaceutically acceptable salt, stereoisomer, solvate, prodrug, isotopic derivative, or tautomer thereof.

In some embodiments, the compound is of Formula (I-A-I-A-c-4):

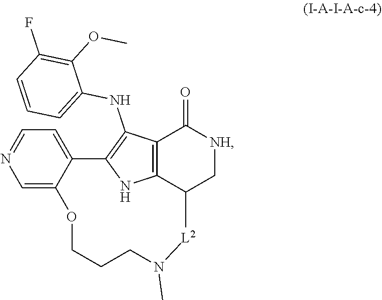

or a pharmaceutically acceptable salt, stereoisomer, solvate, prodrug, isotopic derivative, or tautomer thereof.

In some embodiments, the compound is of Formula (I-A-I-A-c-4′) or (I-A-I-A-c-4″):

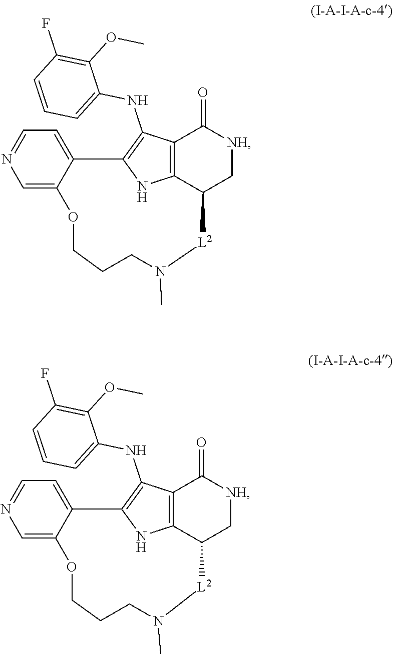

or a pharmaceutically acceptable salt, stereoisomer, solvate, prodrug, isotopic derivative, or tautomer thereof.

In some embodiments, the compound is of Formula (I-A-I-A-c-5):

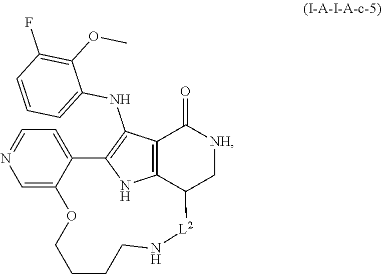

or a pharmaceutically acceptable salt, stereoisomer, solvate, prodrug, isotopic derivative, or tautomer thereof.

In some embodiments, the compound is of Formula (I-A-I-A-c-5′) or (I-A-I-A-c-5″):

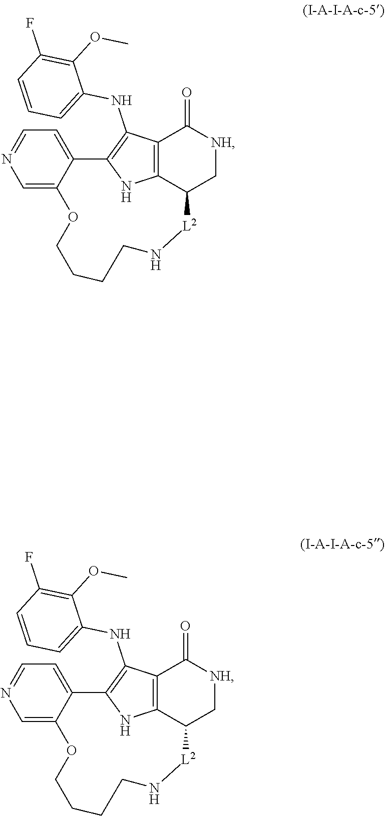

or a pharmaceutically acceptable salt, stereoisomer, solvate, prodrug, isotopic derivative, or tautomer thereof.

In some embodiments, the compound is of Formula (I-A-I-A-c-6):

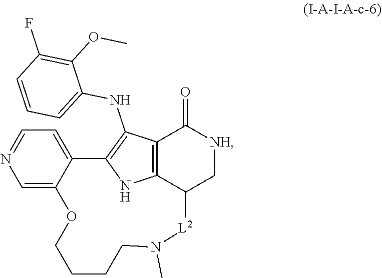

or a pharmaceutically acceptable salt, stereoisomer, solvate, prodrug, isotopic derivative, or tautomer thereof.

In some embodiments, the compound is of Formula (I-A-I-A-c-6′) or (I-A-I-A-c-6″):

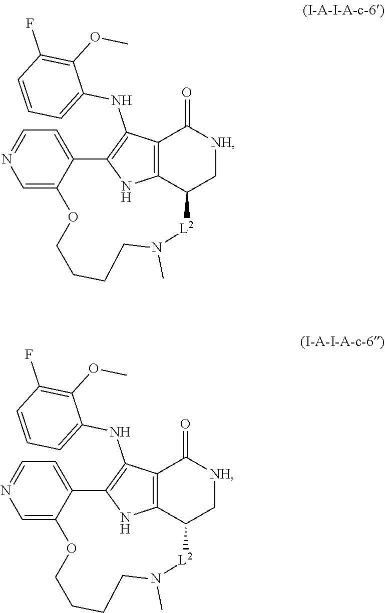

or a pharmaceutically acceptable salt, stereoisomer, solvate, prodrug, isotopic derivative, or tautomer thereof.

In some embodiments, the compound is of Formula (I-A-I-A-c-7):

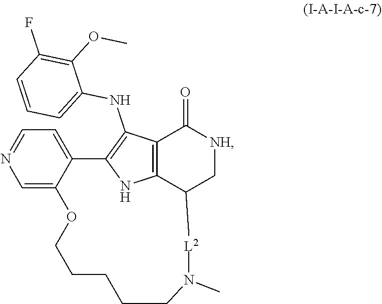

or a pharmaceutically acceptable salt, stereoisomer, solvate, prodrug, isotopic derivative, or tautomer thereof.

In some embodiments, the compound is of Formula (I-A-I-A-c-7′) or (I-A-I-A-c-7″):

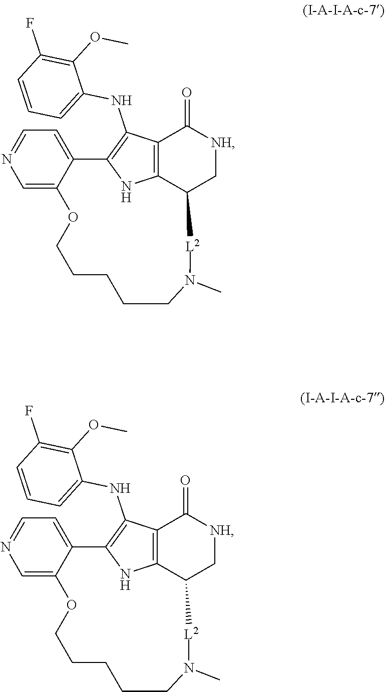

or a pharmaceutically acceptable salt, stereoisomer, solvate, prodrug, isotopic derivative, or tautomer thereof.

In some embodiments, the compound is of Formula (I-A-I-A-d-1):

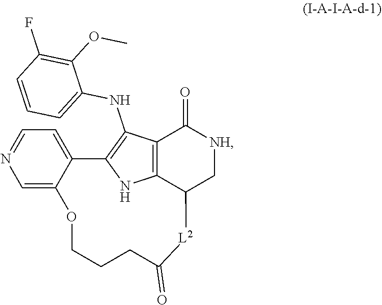

or a pharmaceutically acceptable salt, stereoisomer, solvate, prodrug, isotopic derivative, or tautomer thereof.

In some embodiments, the compound is of Formula (I-A-I-A-d-1′) or (I-A-I-A-d-1″):

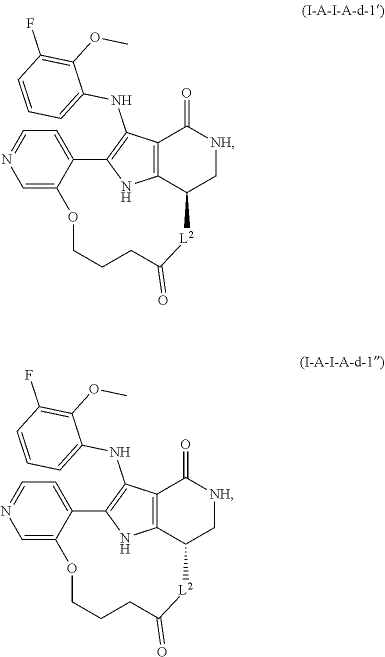

or a pharmaceutically acceptable salt, stereoisomer, solvate, prodrug, isotopic derivative, or tautomer thereof.

In some embodiments, the compound is of Formula (I-A-I-A-d-2):

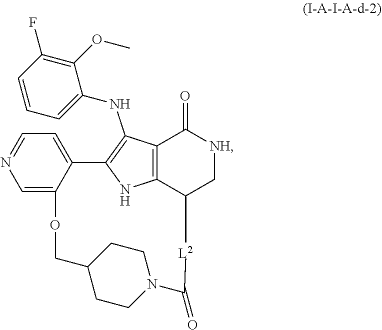

or a pharmaceutically acceptable salt, stereoisomer, solvate, prodrug, isotopic derivative, or tautomer thereof.

In some embodiments, the compound is of Formula (I-A-I-A-d-2′) or (I-A-I-A-d-2″):

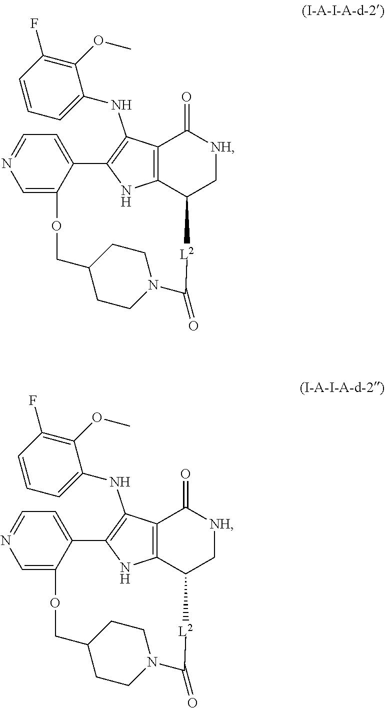

or a pharmaceutically acceptable salt, stereoisomer, solvate, prodrug, isotopic derivative, or tautomer thereof.

In some embodiments, the compound is of Formula (I-A-I-A-d-3):

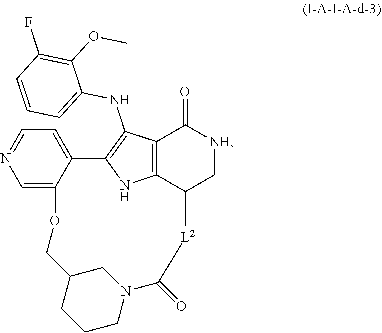

or a pharmaceutically acceptable salt, stereoisomer, solvate, prodrug, isotopic derivative, or tautomer thereof.

In some embodiments, the compound is of Formula (I-A-I-A-d-3′) or (I-A-I-A-d-3″)

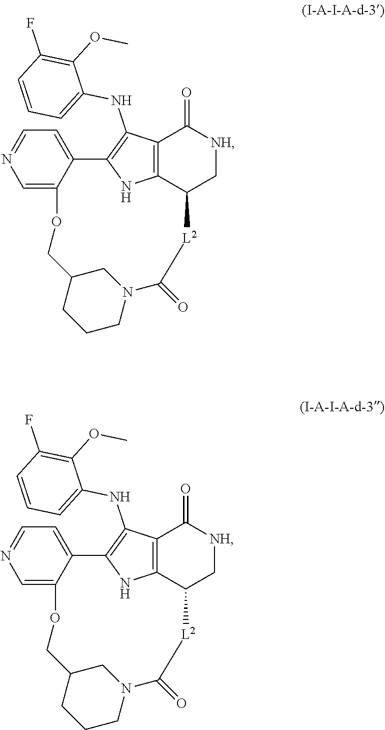

or a pharmaceutically acceptable salt, stereoisomer, solvate, prodrug, isotopic derivative, or tautomer thereof.

In some embodiments, the compound is of Formula (I-A-I-A-d-4):

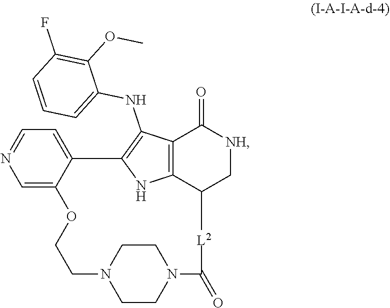

or a pharmaceutically acceptable salt, stereoisomer, solvate, prodrug, isotopic derivative, or tautomer thereof.

In some embodiments, the compound is of Formula (I-A-I-A-d-4′) or (I-A-I-A-d-4″):

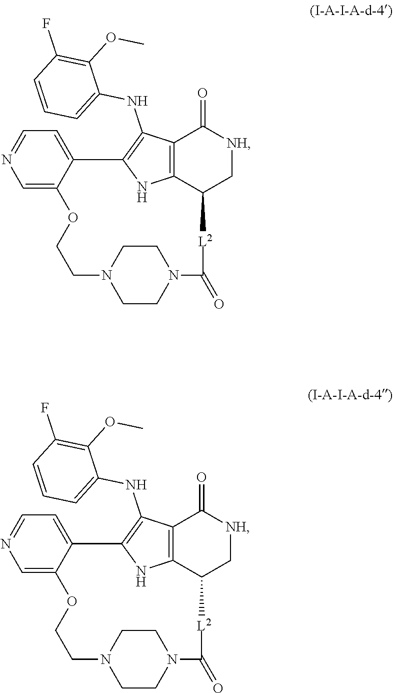

or a pharmaceutically acceptable salt, stereoisomer, solvate, prodrug, isotopic derivative, or tautomer thereof.

In some embodiments, the compound is of Formula (I-A-I-A-d-5):

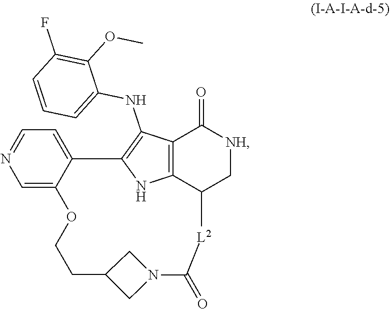

or a pharmaceutically acceptable salt, stereoisomer, solvate, prodrug, isotopic derivative, or tautomer thereof.

In some embodiments, the compound is of Formula (I-A-I-A-d-5′) or (I-A-I-A-d-5″):

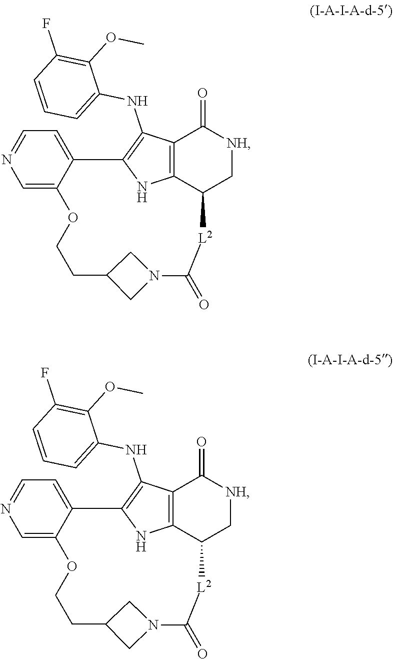

or a pharmaceutically acceptable salt, stereoisomer, solvate, prodrug, isotopic derivative, or tautomer thereof.

In some embodiments, the compound is of Formula (I-A-I-A-e-1):

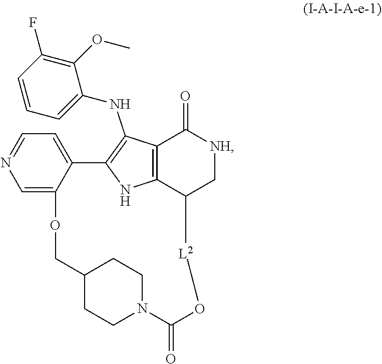

or a pharmaceutically acceptable salt, stereoisomer, solvate, prodrug, isotopic derivative, or tautomer thereof.

In some embodiments, the compound is of Formula (I-A-I-A-e-1′) or (I-A-I-A-e-1″

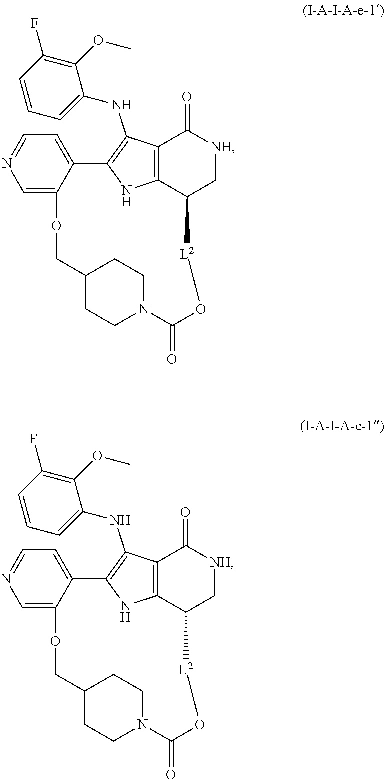

or a pharmaceutically acceptable salt, stereoisomer, solvate, prodrug, isotopic derivative, or tautomer thereof.

In some embodiments, the compound is of Formula (I-A-I-A-e-2):

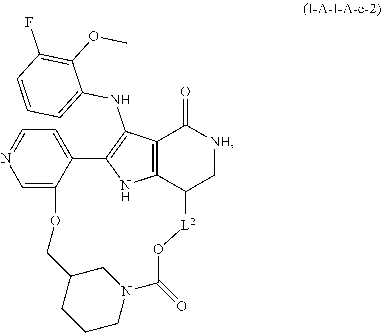

or a pharmaceutically acceptable salt, stereoisomer, solvate, prodrug, isotopic derivative, or tautomer thereof.

In some embodiments, the compound is of Formula (I-A-I-A-e-2′) or (I-A-I-A-e-2″):

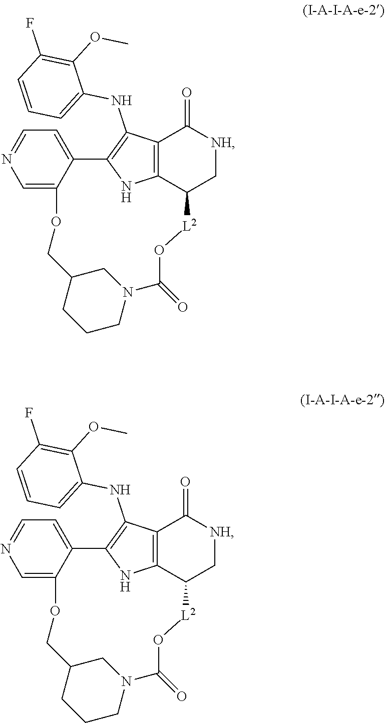

or a pharmaceutically acceptable salt, stereoisomer, solvate, prodrug, isotopic derivative, or tautomer thereof.

In some embodiments, the compound is of Formula (I-A-I-A-f-1):

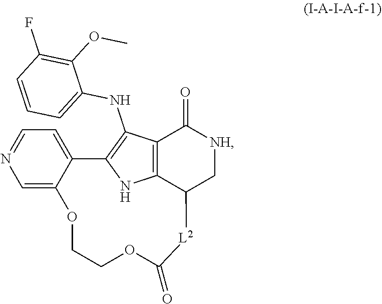

or a pharmaceutically acceptable salt, stereoisomer, solvate, prodrug, isotopic derivative, or tautomer thereof.

In some embodiments, the compound is of Formula (I-A-I-A-f-1′) or (I-A-I-A-f-2″):

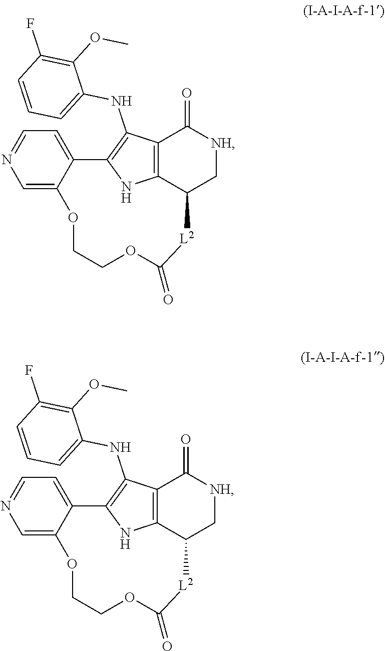

or a pharmaceutically acceptable salt, stereoisomer, solvate, prodrug, isotopic derivative, or tautomer thereof.

In some embodiments, the compound is of Formula (I-A-I-A-g-1):

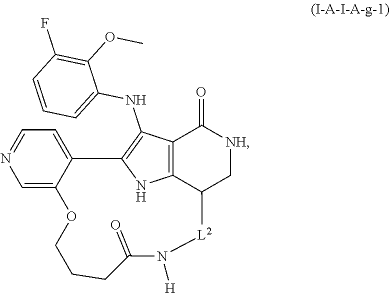

or a pharmaceutically acceptable salt, stereoisomer, solvate, prodrug, isotopic derivative, or tautomer thereof.

In some embodiments, the compound is of Formula (I-A-I-A-g-1′) or (I-A-I-A-g-1″):

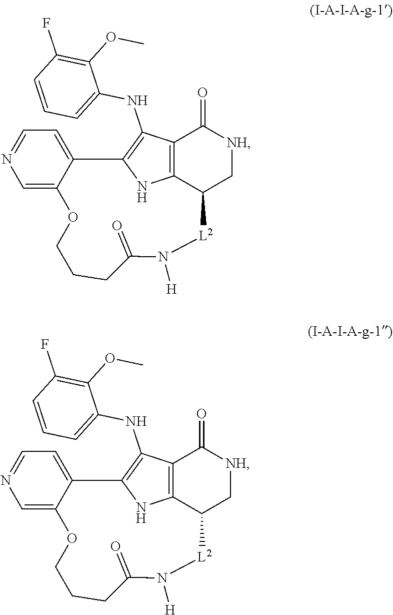

or a pharmaceutically acceptable salt, stereoisomer, solvate, prodrug, isotopic derivative, or tautomer thereof.

In some embodiments, the compound is of Formula (I-A-I-A-g-2):

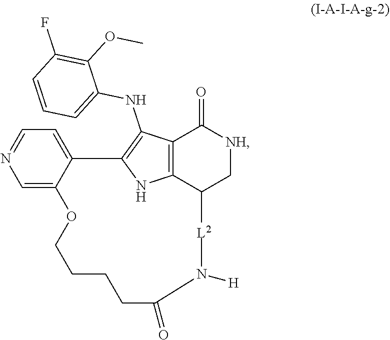

or a pharmaceutically acceptable salt, stereoisomer, solvate, prodrug, isotopic derivative, or tautomer thereof.

In some embodiments, the compound is of Formula (I-A-I-A-g-2′) or (I-A-I-A-g-2″):

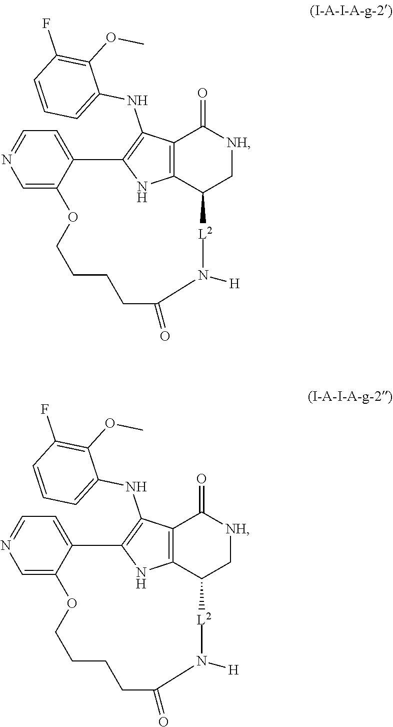

or a pharmaceutically acceptable salt, stereoisomer, solvate, prodrug, isotopic derivative, or tautomer thereof.

In some embodiments, the compound is of Formula (I-A-I-A-h-1):

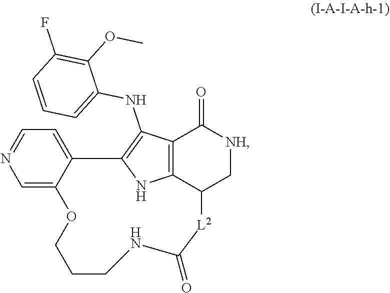

or a pharmaceutically acceptable salt, stereoisomer, solvate, prodrug, isotopic derivative, or tautomer thereof.

In some embodiments, the compound is of Formula (I-A-I-A-h-1′) or (I-A-I-A-h-1″):

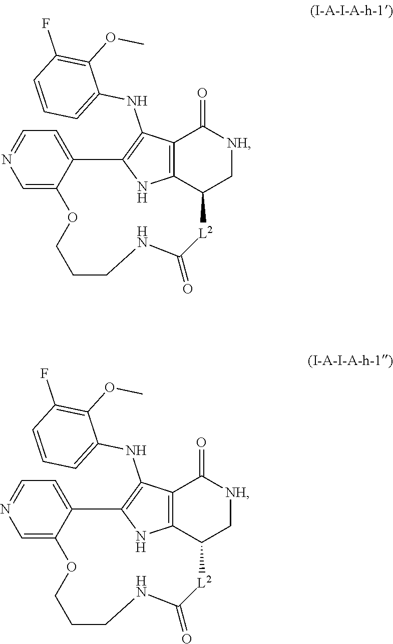

or a pharmaceutically acceptable salt, stereoisomer, solvate, prodrug, isotopic derivative, or tautomer thereof.

In some embodiments, the compound is of Formula (I-A-I-A-h-2):

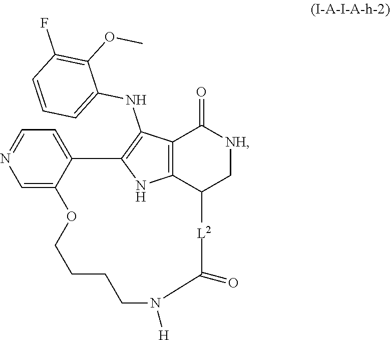

or a pharmaceutically acceptable salt, stereoisomer, solvate, prodrug, isotopic derivative, or tautomer thereof.

In some embodiments, the compound is of Formula (I-A-I-A-h-2′) or (I-A-I-A-h-2″):

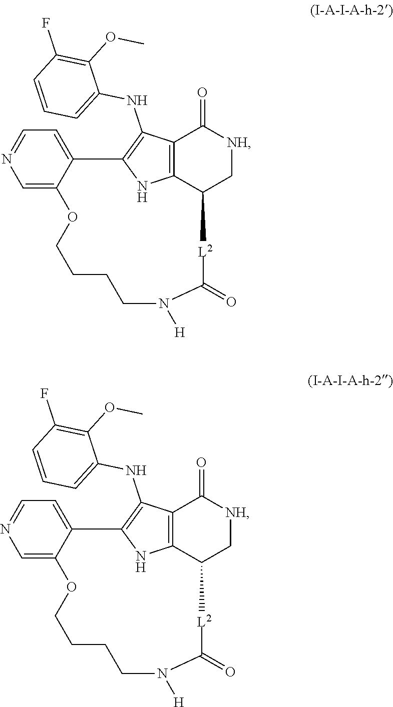

or a pharmaceutically acceptable salt, stereoisomer, solvate, prodrug, isotopic derivative, or tautomer thereof.

In some embodiments, the compound is of Formula (I-A-I-A-100):

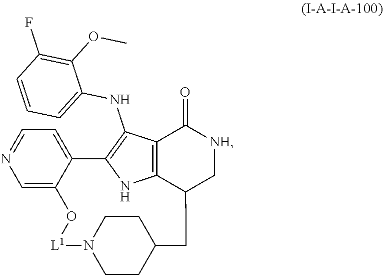

or a pharmaceutically acceptable salt, stereoisomer, solvate, prodrug, isotopic derivative, or tautomer thereof.

In some embodiments, the compound is of Formula (I-A-I-A-101):

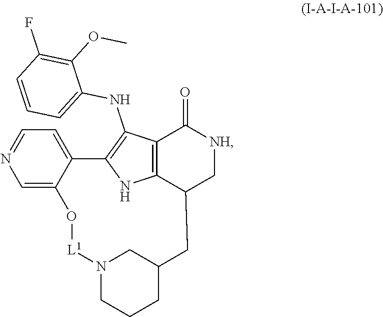

or a pharmaceutically acceptable salt, stereoisomer, solvate, prodrug, isotopic derivative, or tautomer thereof.

In some embodiments, the compound is of Formula (I-A-I-A-102):

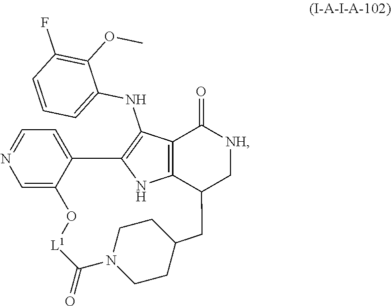

or a pharmaceutically acceptable salt, stereoisomer, solvate, prodrug, isotopic derivative, or tautomer thereof.

In some embodiments, the compound is of Formula (I-A-I-A-103):

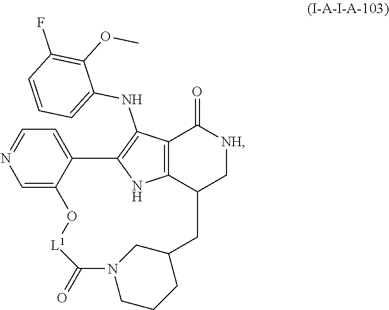

or a pharmaceutically acceptable salt, stereoisomer, solvate, prodrug, isotopic derivative, or tautomer thereof.

In some embodiments, the compound is of Formula (I-A-I-A-104):

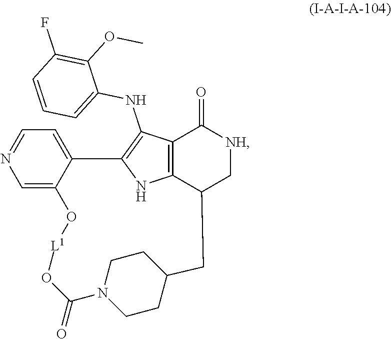

or a pharmaceutically acceptable salt, stereoisomer, solvate, prodrug, isotopic derivative, or tautomer thereof.

In some embodiments, the compound is of Formula (I-A-I-A-105):

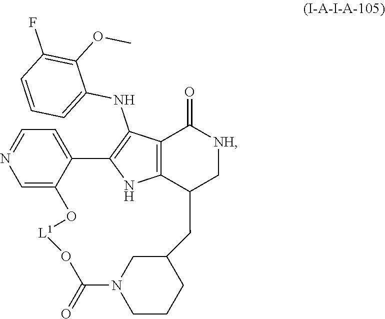

or a pharmaceutically acceptable salt, stereoisomer, solvate, prodrug, isotopic derivative, or tautomer thereof.

In some embodiments, the compound is of Formula (I-A-I-A-106):

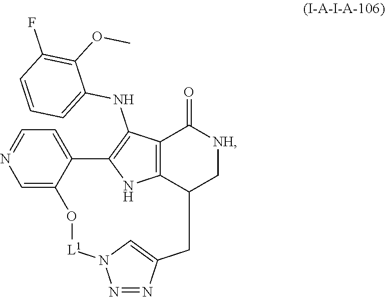

or a pharmaceutically acceptable salt, stereoisomer, solvate, prodrug, isotopic derivative, or tautomer thereof.

In some embodiments, the compound is of Formula (I-A-I-A-107):

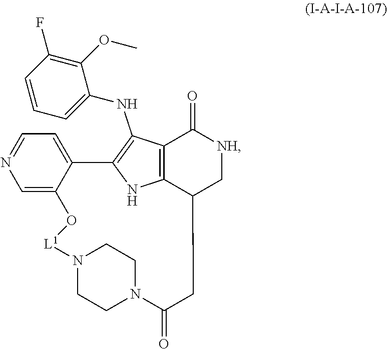

or a pharmaceutically acceptable salt, stereoisomer, solvate, prodrug, isotopic derivative, or tautomer thereof.

In some embodiments, the compound is of Formula (I-A-I-A-108):

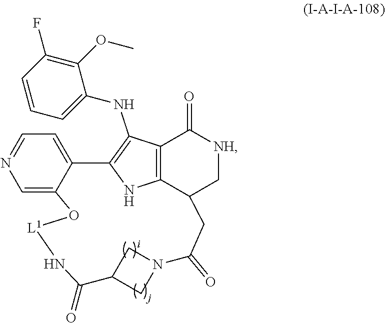

or a pharmaceutically acceptable salt, stereoisomer, solvate, prodrug, isotopic derivative, or tautomer thereof, wherein i is an integer selected from 0, 1, 2, and 3; j is an integer selected from 1, 2, 3, 4 and 5; and all other variables are as defined herein.

In some embodiments, the compound is of Formula (I-A-I-A-109):

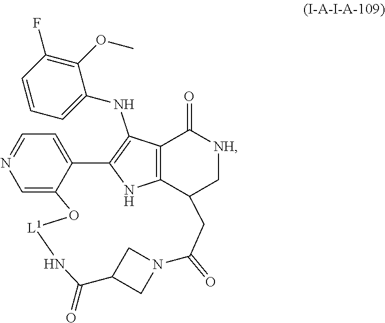

or a pharmaceutically acceptable salt, stereoisomer, solvate, prodrug, isotopic derivative, or tautomer thereof.

In some embodiments, the compound is of Formula (I-A-I-A-110):

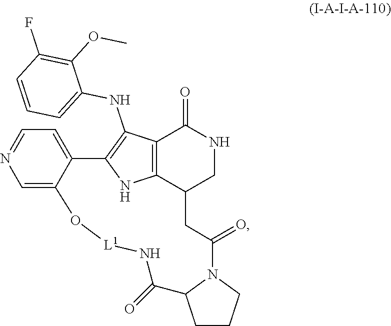

or a pharmaceutically acceptable salt, stereoisomer, solvate, prodrug, isotopic derivative, or tautomer thereof.

In some embodiments, the compound is of Formula (I-A-I-A-110′), (I-A-I-A-110″), (I-A-I-A-110″′), or (I-A-I-A-110″″):

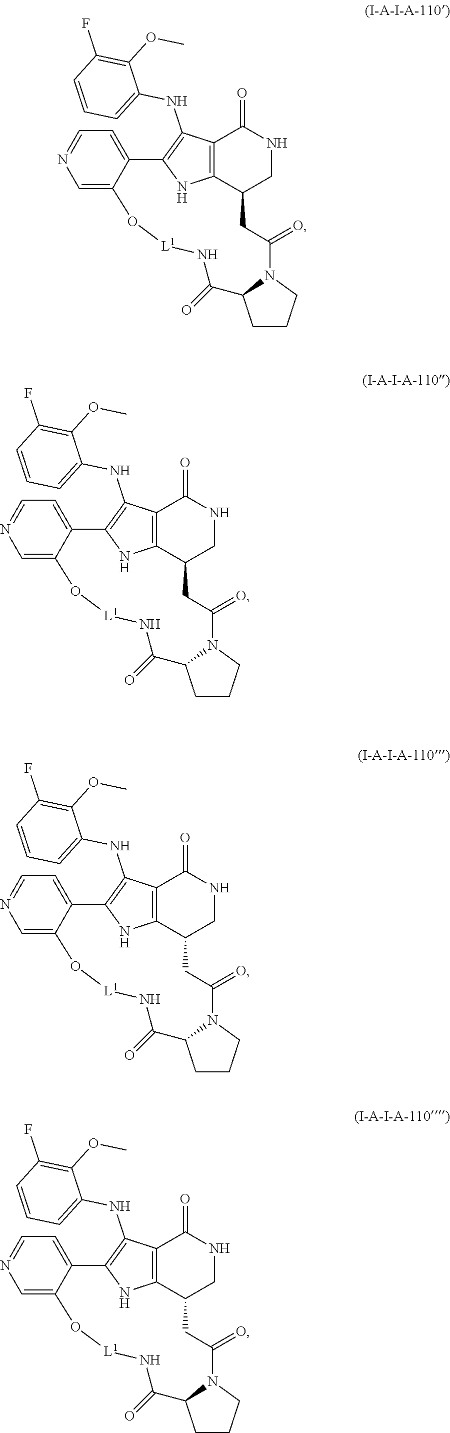

or a pharmaceutically acceptable salt, stereoisomer, solvate, prodrug, isotopic derivative, or tautomer thereof.

In some embodiments, the compound is of Formula (I-A-I-A-111).

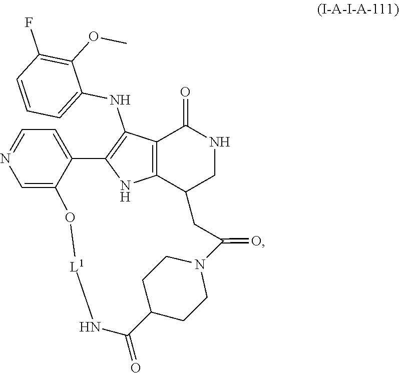

or a pharmaceutically acceptable salt, stereoisomer, solvate, prodrug, isotopic derivative, or tautomer thereof.

In some embodiments, the compound is of Formula (I-A-I-B-a-1):

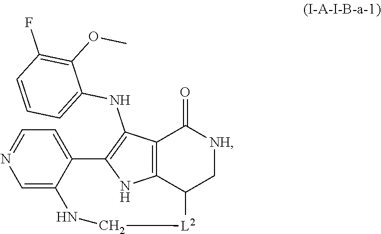

or a pharmaceutically acceptable salt, stereoisomer, solvate, prodrug, isotopic derivative, or tautomer thereof.

In some embodiments, the compound is of Formula (I-A-I-B-a-1′) or (I-A-I-B-a-1″):

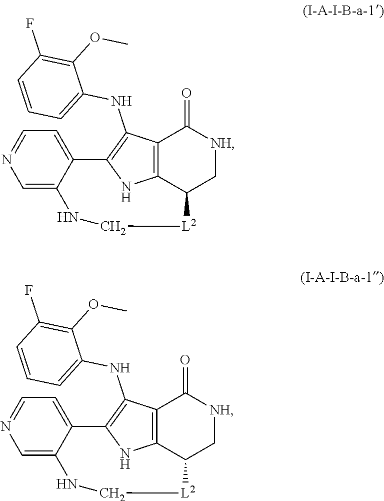

or a pharmaceutically acceptable salt, stereoisomer, solvate, prodrug, isotopic derivative, or tautomer thereof.

In some embodiments, the compound is of Formula (I-A-I-B-a-2):

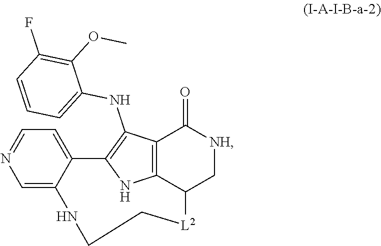

or a pharmaceutically acceptable salt, stereoisomer, solvate, prodrug, isotopic derivative, or tautomer thereof.

In some embodiments, the compound is of Formula (I-A-I-B-a-2′) or (I-A-I-B-a-2″):

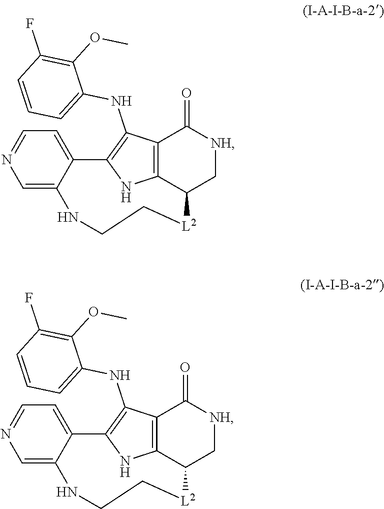

or a pharmaceutically acceptable salt, stereoisomer, solvate, prodrug, isotopic derivative, or tautomer thereof.

In some embodiments, the compound is of Formula (I-A-I-B-a-3):

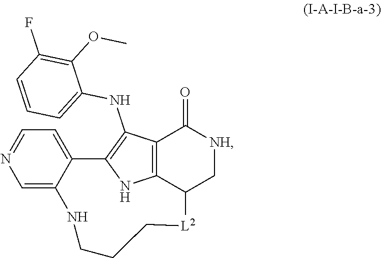

or a pharmaceutically acceptable salt, stereoisomer, solvate, prodrug, isotopic derivative, or tautomer thereof.

In some embodiments, the compound is of Formula (I-A-I-B-a-3′ r (I-A-I-B-a-3″):

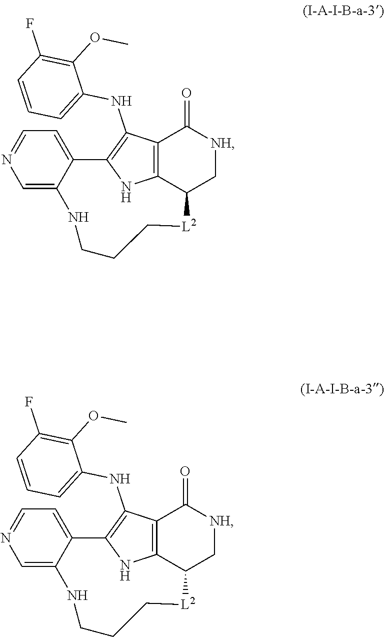

or a pharmaceutically acceptable salt, stereoisomer, solvate, prodrug, isotopic derivative, or tautomer thereof.

In some embodiments, the compound is of Formula (I-A-I-B-a-4):

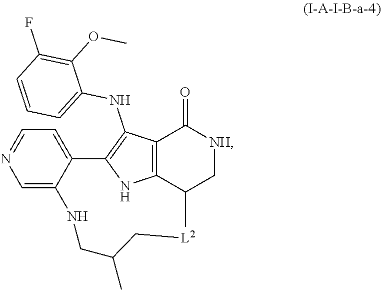

or a pharmaceutically acceptable salt, stereoisomer, solvate, prodrug, isotopic derivative, or tautomer thereof.

In some embodiments, the compound is of Formula (I-A-I-B-a-4′) or (I-A-I-B-a-4″):

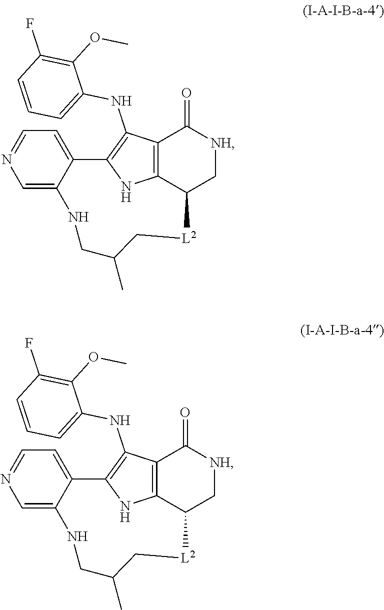

or a pharmaceutically acceptable salt, stereoisomer, solvate, prodrug, isotopic derivative, or tautomer thereof.

In some embodiments, the compound is of Formula (I-A-I-B-a-5):

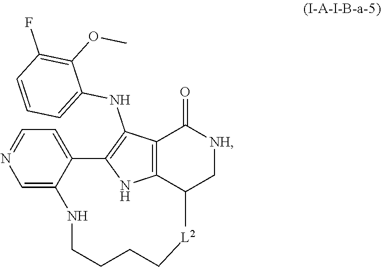

or a pharmaceutically acceptable salt, stereoisomer, solvate, prodrug, isotopic derivative, or tautomer thereof.

In some embodiments, the compound is of Formula (I-A-I-B-a-5′) or (I-A-I-B-a-5″):

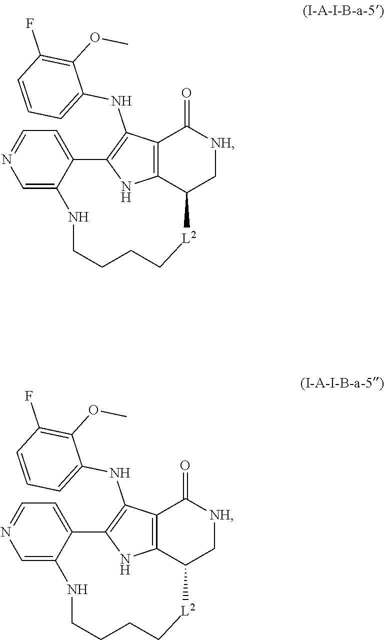

or a pharmaceutically acceptable salt, stereoisomer, solvate, prodrug, isotopic derivative, or tautomer thereof.

In some embodiments, the compound is of Formula (I-A-I-B-a-6):

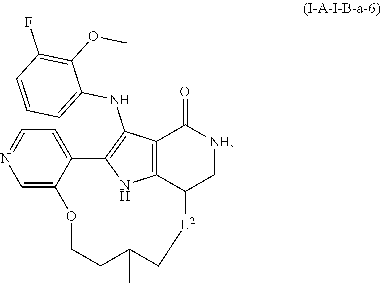

or a pharmaceutically acceptable salt, stereoisomer, solvate, prodrug, isotopic derivative, or tautomer thereof.

In some embodiments, the compound is of Formula (I-A-I-B-a-6′) or (I-A-I-B-a-6″):

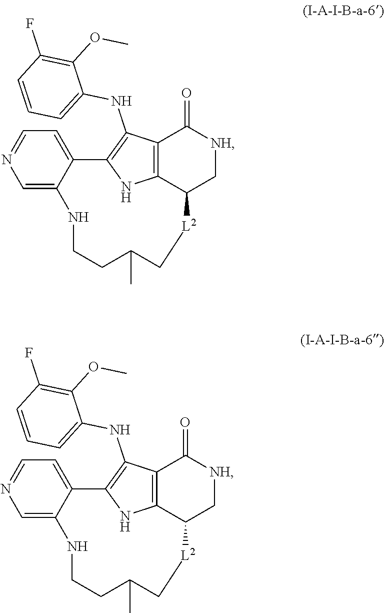

or a pharmaceutically acceptable salt, stereoisomer, solvate, prodrug, isotopic derivative, or tautomer thereof.

In some embodiments, the compound is of Formula (I-A-I-B-a-7):

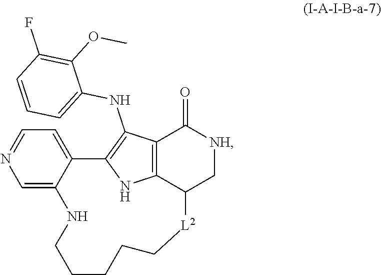

or a pharmaceutically acceptable salt, stereoisomer, solvate, prodrug, isotopic derivative, or tautomer thereof.

In some embodiments, the compound is of Formula (I-A-I-B-a-7′) or (I-A-I-B-a-7″):

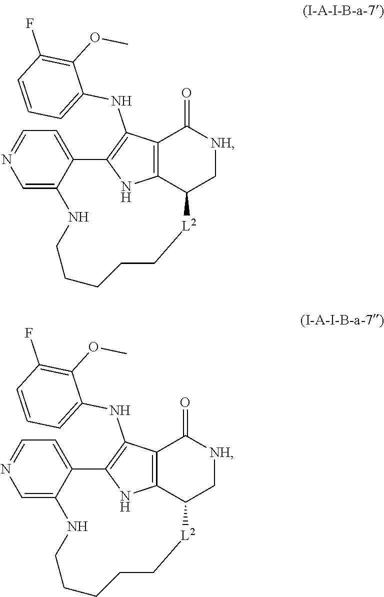

or a pharmaceutically acceptable salt, stereoisomer, solvate, prodrug, isotopic derivative, or tautomer thereof.

In some embodiments, the compound is of Formula (I-A-I-B-a-8):

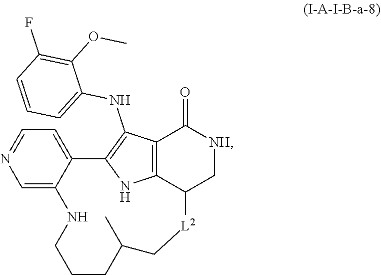

or a pharmaceutically acceptable salt, stereoisomer, solvate, prodrug, isotopic derivative, or tautomer thereof.

In some embodiments, the compound is of Formula (I-A-I-B-a-8′) or (I-A-I-B-a-8″):

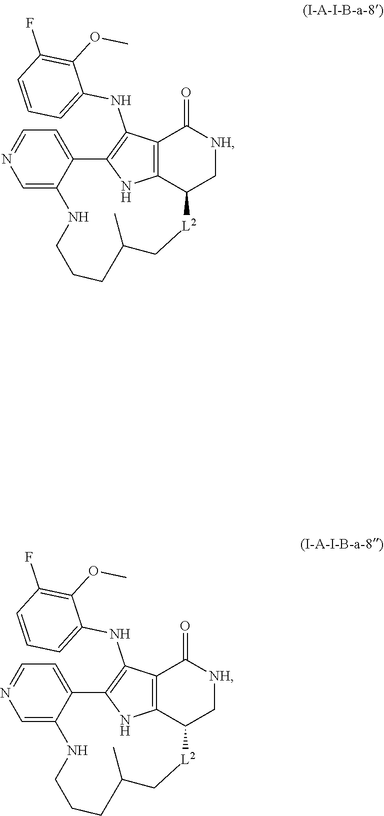

or a pharmaceutically acceptable salt, stereoisomer, solvate, prodrug, isotopic derivative, or tautomer thereof.

In some embodiments, the compound is of Formula (I-A-I-B-a-9):

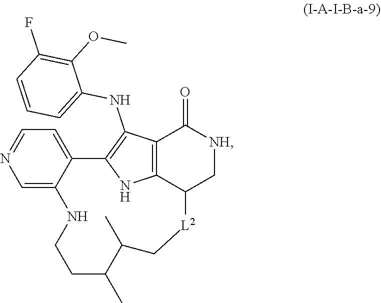

or a pharmaceutically acceptable salt, stereoisomer, solvate, prodrug, isotopic derivative, or tautomer thereof.

In some embodiments, the compound is of Formula (I-A-I-B-a-9′) or (I-A-I-B-a-9″):

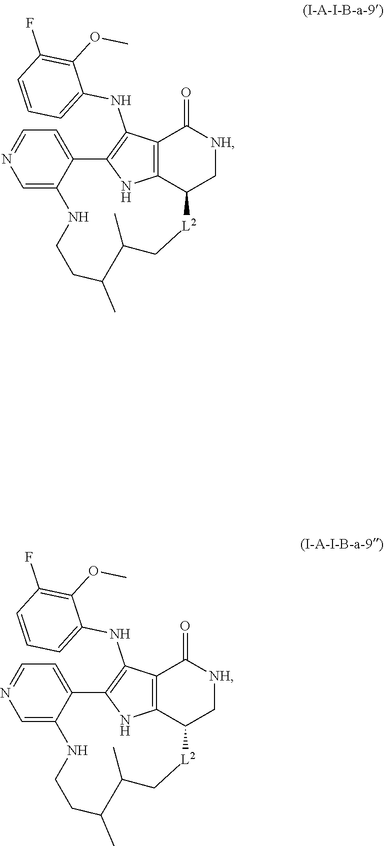

or a pharmaceutically acceptable salt, stereoisomer, solvate, prodrug, isotopic derivative, or tautomer thereof.

In some embodiments, the compound is of Formula (I-A-I-B-a-10):

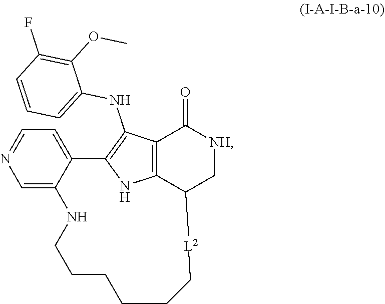

or a pharmaceutically acceptable salt, stereoisomer, solvate, prodrug, isotopic derivative, or tautomer thereof.

In some embodiments, the compound is of Formula (I-A-I-B-a-10′) or (I-A-I-B-a-10″):

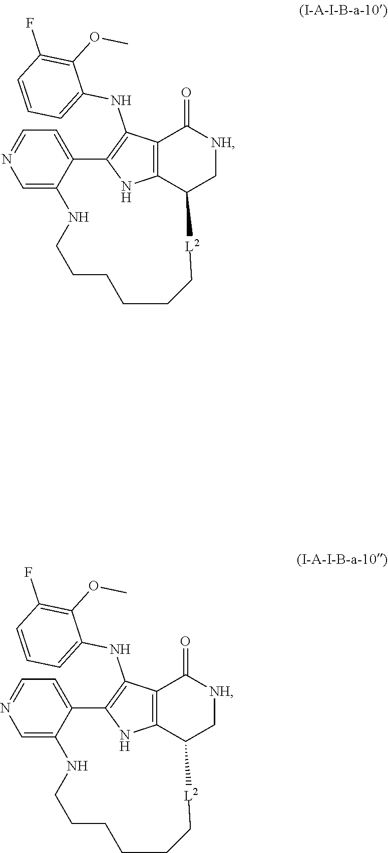

or a pharmaceutically acceptable salt, stereoisomer, solvate, prodrug, isotopic derivative, or tautomer thereof.

In some embodiments, the compound is of Formula (I-A-I-B-a-11):

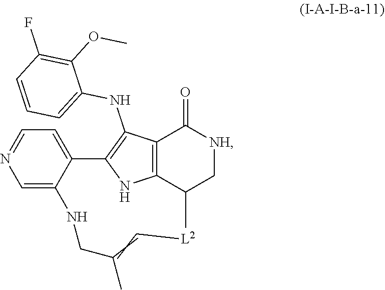

or a pharmaceutically acceptable salt, stereoisomer, solvate, prodrug, isotopic derivative, or tautomer thereof.

In some embodiments, the compound is of Formula (E-I-A-I-B-a-11) or (Z-I-A-I-B-a-11):

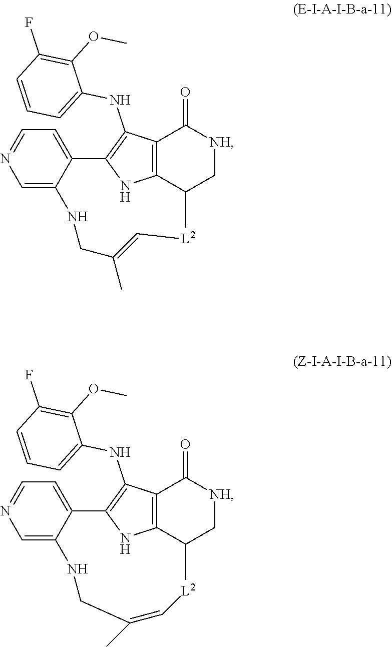

or a pharmaceutically acceptable salt, stereoisomer, solvate, prodrug, isotopic derivative, or tautomer thereof.

In some embodiments, the compound is of Formula (E-I-A-I-B-a-11′), (Z-I-A-I-B-a-11′), (E-I-A-I-B-a-11″), or (Z-I-A-I-B-a-11″):

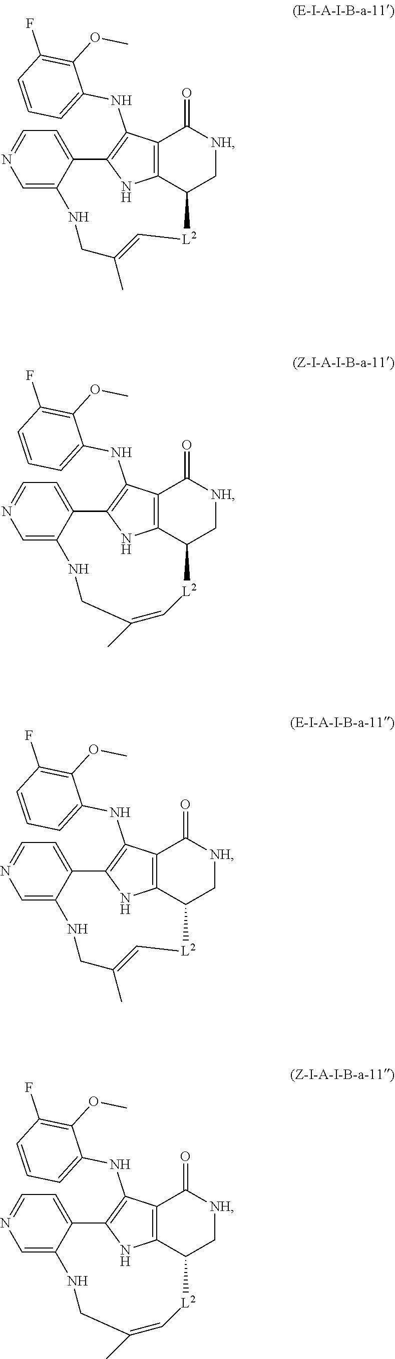

or a pharmaceutically acceptable salt, stereoisomer, solvate, prodrug, isotopic derivative, or tautomer thereof.

In some embodiments, the compound is of Formula (I-A-I-B-a-12):

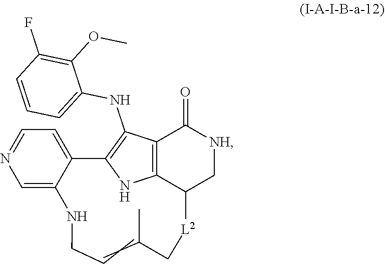

or a pharmaceutically acceptable salt, stereoisomer, solvate, prodrug, isotopic derivative, or tautomer thereof.

In some embodiments, the compound is of Formula (E-I-A-I-B-a-12) or (Z-I-A-I-B-a-12):

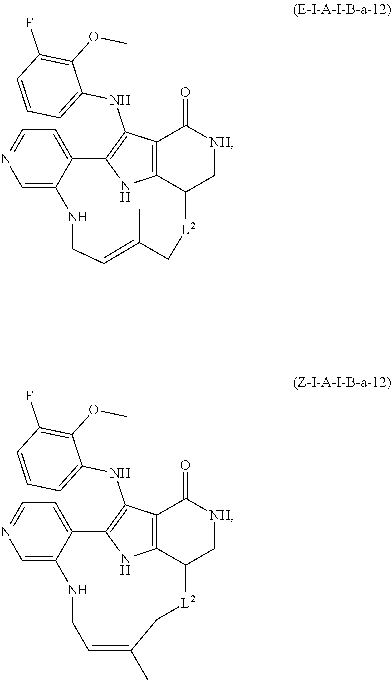

or a pharmaceutically acceptable salt, stereoisomer, solvate, prodrug, isotopic derivative, or tautomer thereof.

In some embodiments, the compound is of Formula (E-I-A-I-B-a-12′), (E-I-A-I-B-a-12″), (Z-I-A-B-a-12′), or (Z-I-A-1-B-a-12″):

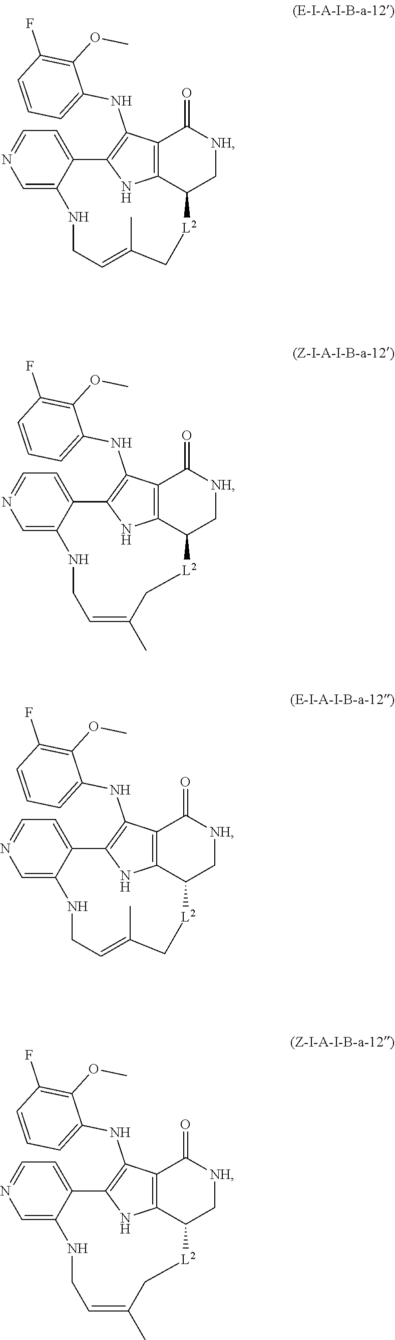

or a pharmaceutically acceptable salt, stereoisomer, solvate, prodrug, isotopic derivative, or tautomer thereof.

In some embodiments, the compound is of Formula (I-A-I-B-a-13):

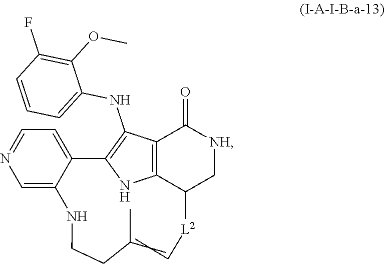

or a pharmaceutically acceptable salt, stereoisomer, solvate, prodrug, isotopic derivative, or tautomer thereof.

In some embodiments, the compound is of Formula (E-I-A-I-B-a-13) or (Z-I-A-I-B-a-13):

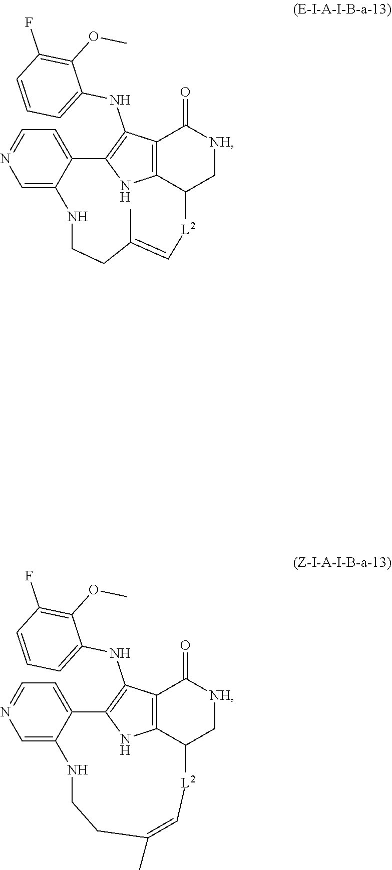

or a pharmaceutically acceptable salt, stereoisomer, solvate, prodrug, isotopic derivative, or tautomer thereof.

In some embodiments, the compound is of Formula (E-I-A-I-B-a-13′), (E-I-A-I-B-a-13″), (Z-I-A-I-B-a-13′), or (Z-I-A-I-B-a-13″):

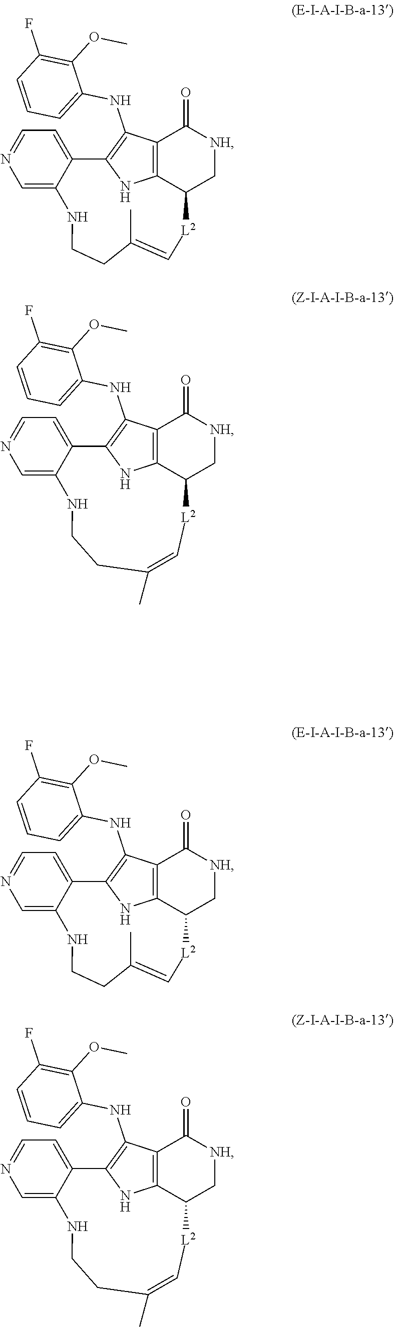

or a pharmaceutically acceptable salt, stereoisomer, solvate, prodrug, isotopic derivative, or tautomer thereof.

In some embodiments, the compound is of Formula (I-A-I-B-a-14):

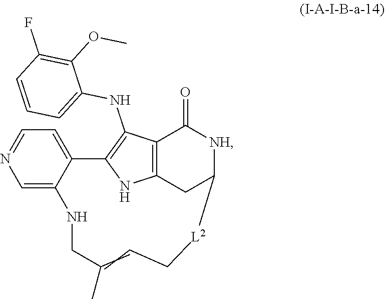

or a pharmaceutically acceptable salt, stereoisomer, solvate, prodrug, isotopic derivative, or tautomer thereof.

In some embodiments, the compound is of Formula (E-I-A-I-B-a-14) or (Z-I-A-I-B-a-14):

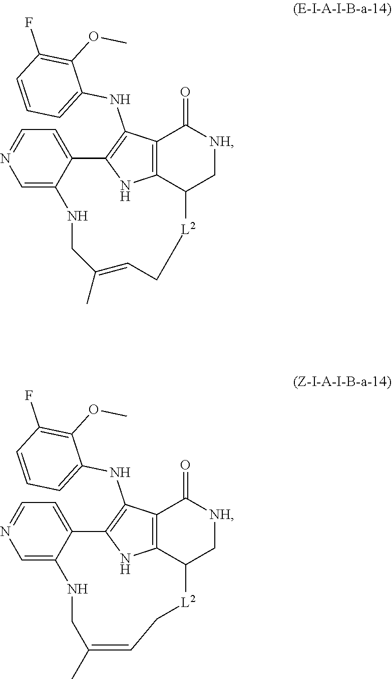

or a pharmaceutically acceptable salt, stereoisomer, solvate, prodrug, isotopic derivative, or tautomer thereof.

In some embodiments, the compound is of Formula (E-I-A-I-B-a-14′), (E-I-A-I-B-a-14″), —(Z-I-A-I-B-a-14′), or (Z-I-A-I-B-a-14″):

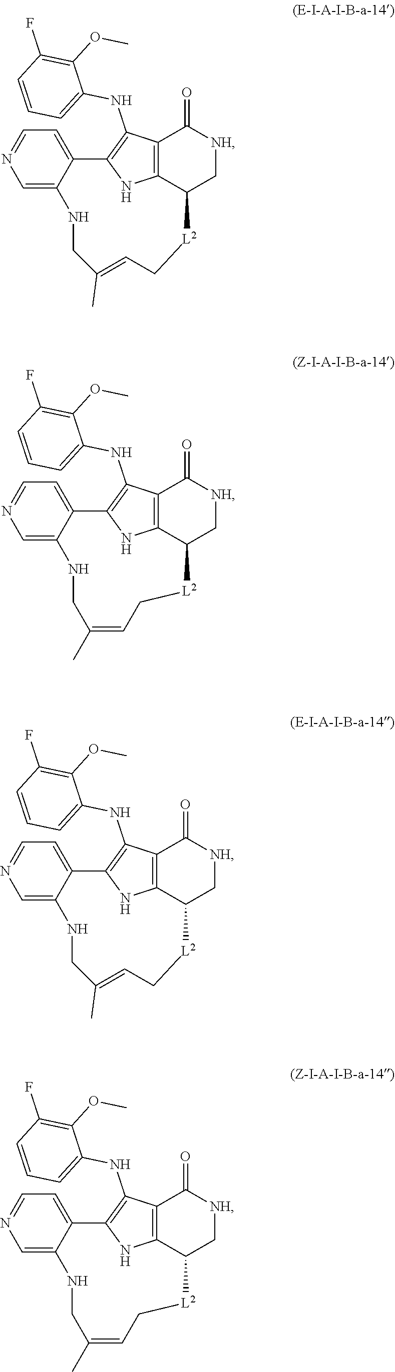

or a pharmaceutically acceptable salt, stereoisomer, solvate, prodrug, isotopic derivative, or tautomer thereof.

In some embodiments, the compound is of Formula (I-A-I-B-a-15):

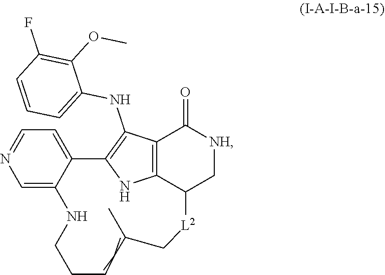

or a pharmaceutically acceptable salt, stereoisomer, solvate, prodrug, isotopic derivative, or tautomer thereof.

In some embodiments, the compound is of Formula (E-I-A-I-B-a-15) or (Z-I-A-I-B-a-15):

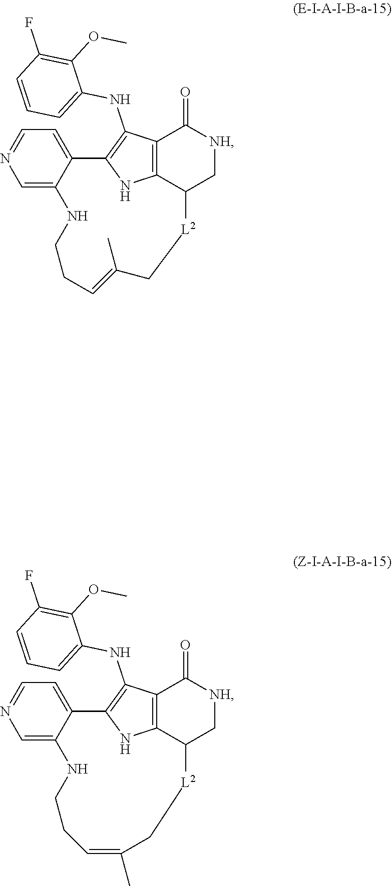

or a pharmaceutically acceptable salt, stereoisomer, solvate, pro rug, isotopic derivative, or tautomer thereof.

In some embodiments, the compound is of Formula (E-I-A-I-B-a-15′), (E-I-A-I-B-a-15″), (Z-I-A-I-B-a-15′), or (Z-I-A-I-B-a-15″):

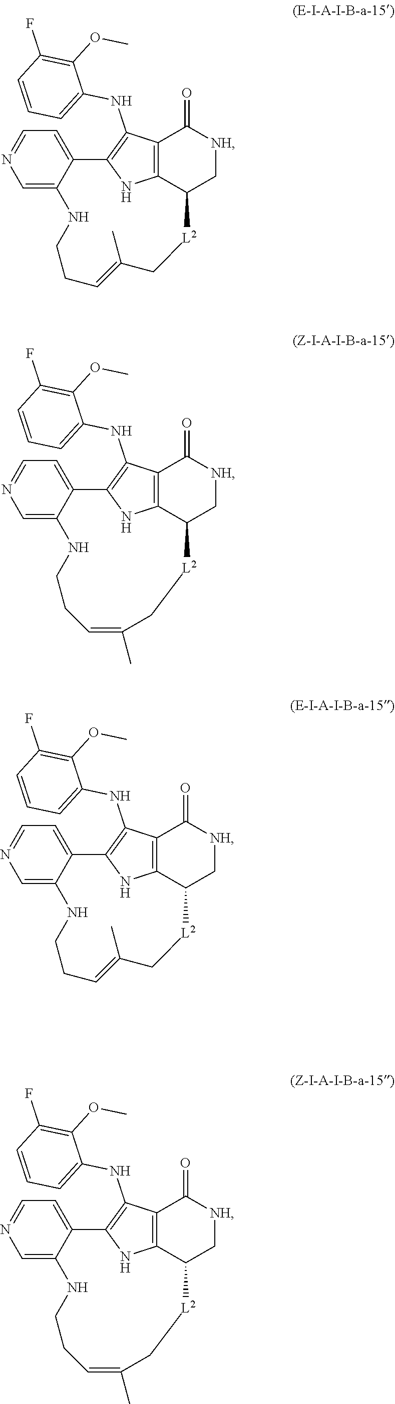

or a pharmaceutically acceptable salt, stereoisomer, solvate, prodrug, isotopic derivative, or tautomer thereof.

In some embodiments, the compound is of Formula (I-A-I-B-a-16):

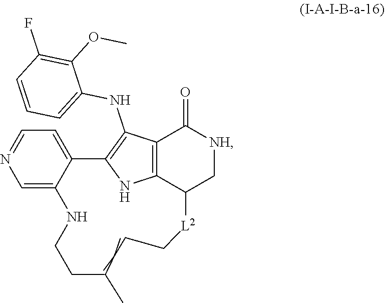

or a pharmaceutically acceptable salt, stereoisomer, solvate, prodrug, isotopic derivative, or tautomer thereof.

In some embodiments, the compound is of Formula (E-I-A-I-B-a-16) or (Z-I-A-I-B-a-16):

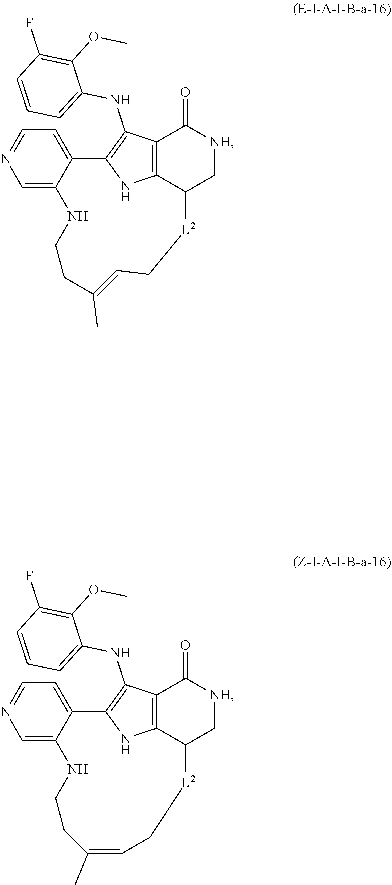

or a pharmaceutically acceptable salt, stereoisomer, solvate, prodrug, isotopic derivative, or tautomer thereof.

In some embodiments, the compound is of Formula (E-I-A-I-B-a-16′), (E-I-A-I-B-a-16″), (Z-I-A-I-B-a-16′), or (Z-I-A-I-B-a-16″):

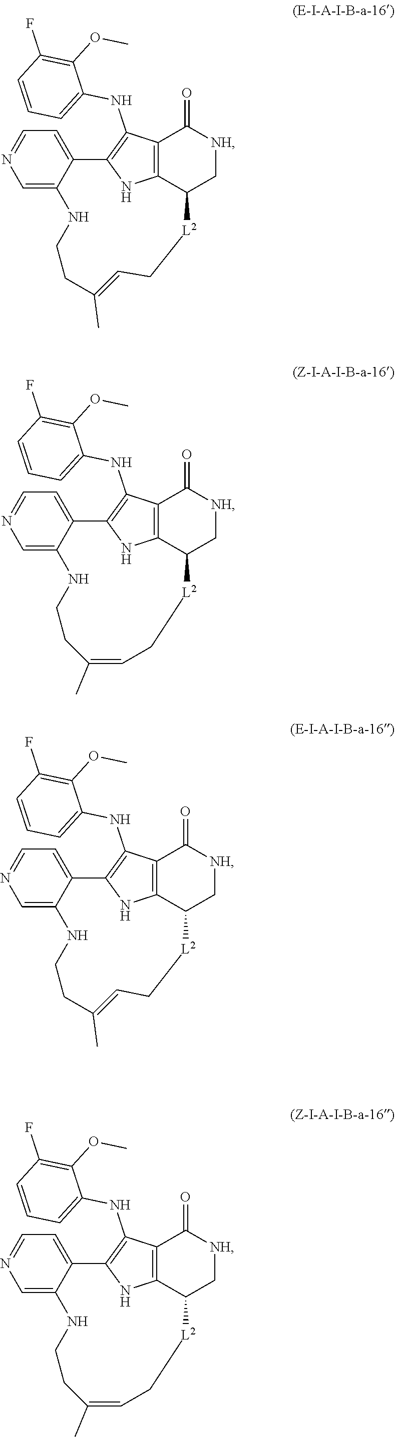

or a pharmaceutically acceptable salt, stereoisomer, solvate, prodrug, isotopic derivative, or tautomer thereof.

In some embodiments, the compound is of Formula (I-A-I-B-a-17):

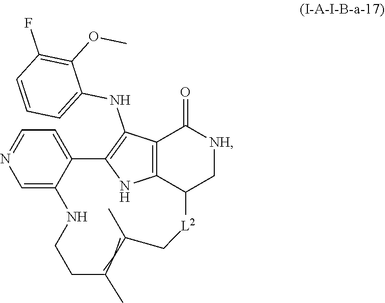

or a pharmaceutically acceptable salt, stereoisomer, solvate, prodrug, isotopic derivative, or tautomer thereof.

In some embodiments, the compound is of Formula (E-I-A-I-B-a-17) or (E-I-A-I-B-a-17):

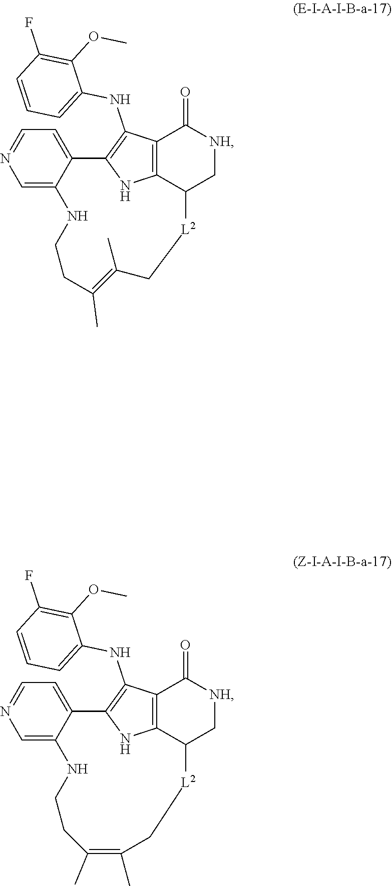

or a pharmaceutically acceptable salt, stereoisomer, solvate, prodrug, isotopic derivative, or tautomer thereof.

In some embodiments, the compound is of Formula (E-I-A-I-B-a-17′), (E-I-A-I-B-a-17″), (Z-I-A-I-B-a-17′), or (Z-I-A-I-B-a-17″):

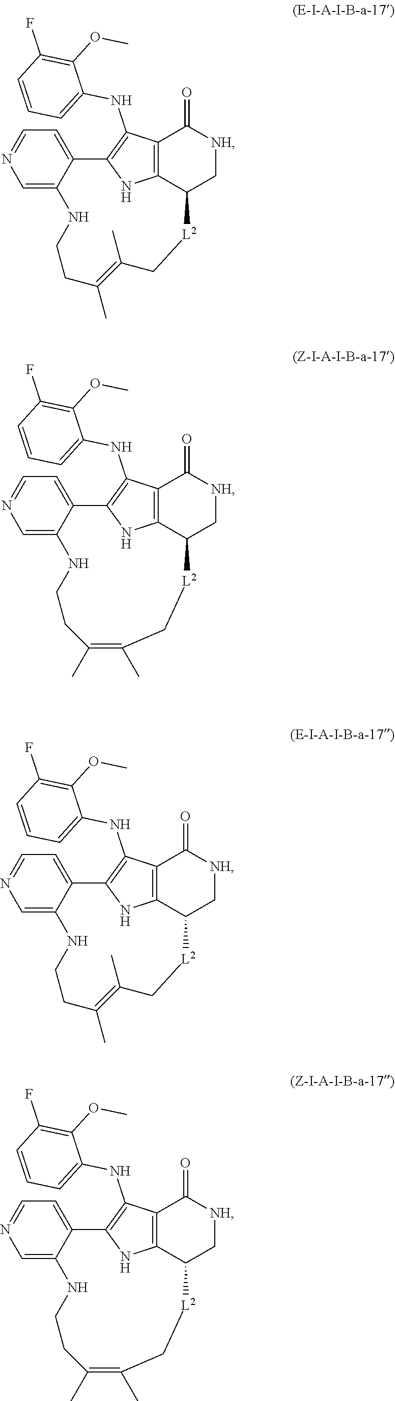

or a pharmaceutically acceptable salt, stereoisomer, solvate, prodrug, isotopic derivative, or tautomer thereof.

In some embodiments, the compound is of Formula (I-A-I-B-a-18):

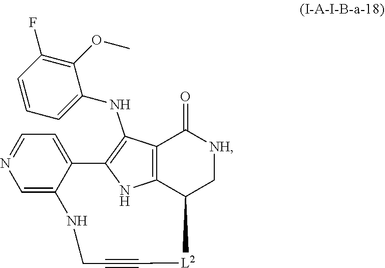

or a pharmaceutically acceptable salt, stereoisomer, solvate, prodrug, isotopic derivative, or tautomer thereof.

In some embodiments, the compound is of Formula (I-A-I-B-a-18′) or (I-A-I-B-a-18″):

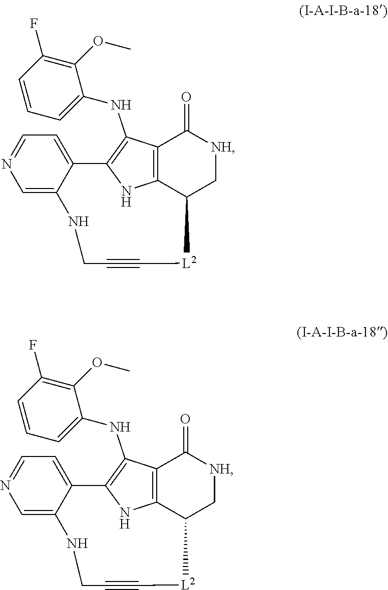

or a pharmaceutically acceptable salt, stereoisomer, solvate, prodrug, isotopic derivative, or tautomer thereof.

In some embodiments, the compound is of Formula (I-A-I-B-a-19):

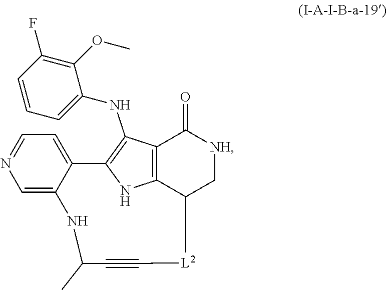

or a pharmaceutically acceptable salt, stereoisomer, solvate, prodrug, isotopic derivative, or tautomer thereof.

In some embodiments, the compound is of Formula (I-A-I-B-a-19′) or (I-A-I-B-a-19″):

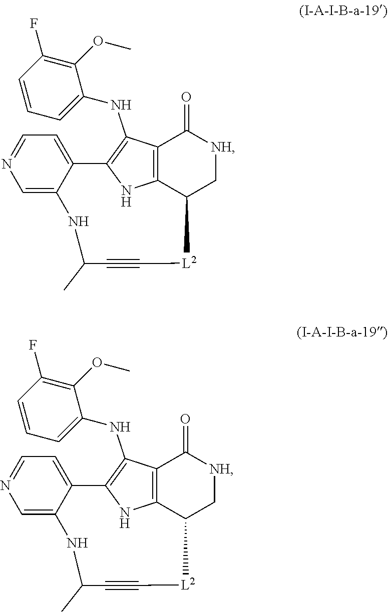

or a pharmaceutically acceptable salt, stereoisomer, solvate, prodrug, isotopic derivative, or tautomer thereof.

In some embodiments, the compound is of Formula (I-A-I-B-a-20):

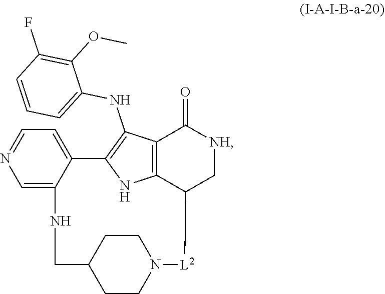

or a pharmaceutically acceptable salt, stereoisomer, solvate, prodrug, isotopic derivative, or tautomer thereof.

In some embodiments, the compound is of Formula (I-A-I-B-a-20′) or (I-A-I-B-a-20″):

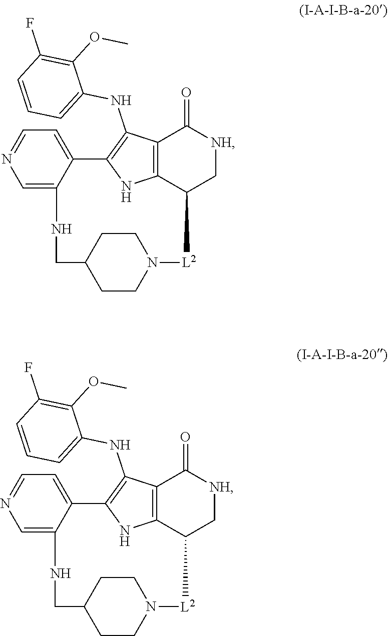

or a pharmaceutically acceptable salt, stereoisomer, solvate, prodrug, isotopic derivative, or tautomer thereof.

In some embodiments, the compound is of Formula (I-A-I-B-a-21):

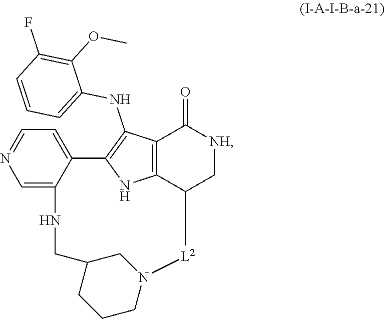

or a pharmaceutically acceptable salt, stereoisomer, solvate, prodrug, isotopic derivative, or tautomer thereof.

In some embodiments, the compound is of Formula (I-A-I-B-a-21′) or (I-A-I-B-a-21″

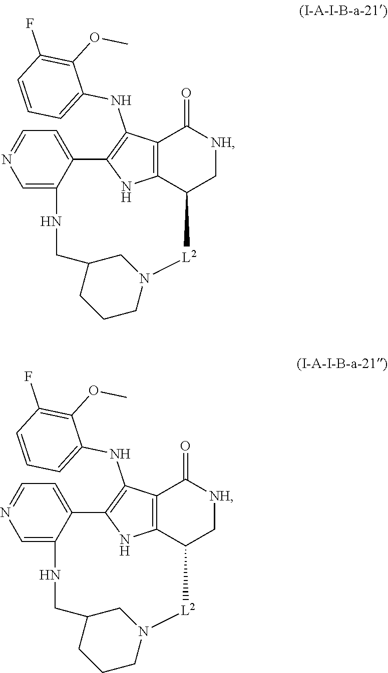

or a pharmaceutically acceptable salt, stereoisomer, solvate, prodrug, isotopic derivative, or tautomer thereof.

In some embodiments, the compound is of Formula (I-A-I-B-a-22):

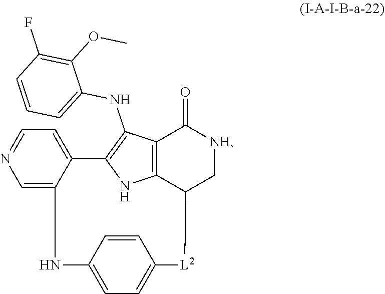

or a pharmaceutically acceptable salt, stereoisomer, solvate, prodrug, isotopic derivative, or tautomer thereof.

In some embodiments, the compound is of Formula (I-A-I-B-a-22′) or (I-A-I-B-a-22″):

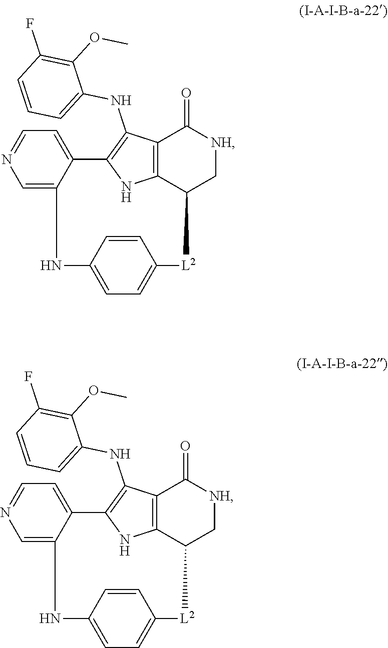

or a pharmaceutically acceptable salt, stereoisomer, solvate, prodrug, isotopic derivative, or tautomer thereof.

In some embodiments, the compound is of Formula (I-A-I-B-a-23):

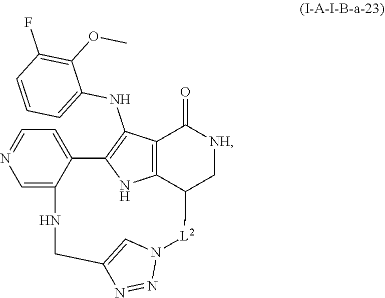

or a pharmaceutically acceptable salt, stereoisomer, solvate, prodrug, isotopic derivative, or tautomer thereof.

In some embodiments, the compound is of Formula (I-A-I-B-a-23′) or (I-A-I-B-a-23″):

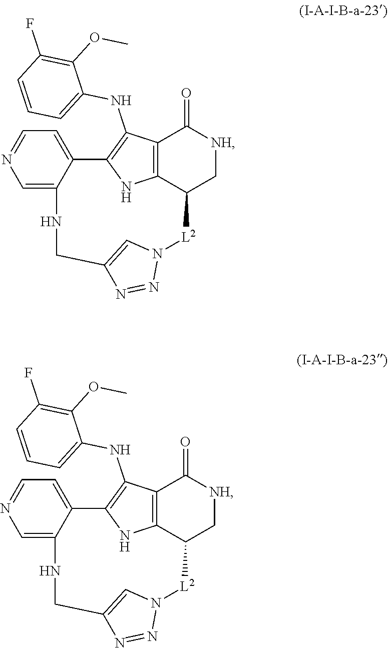

or a pharmaceutically acceptable salt, stereoisomer, solvate, prodrug, isotopic derivative, or tautomer thereof.

In some embodiments, the compound is of Formula (I-A-I-B-a-24):

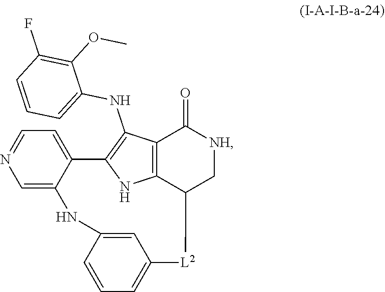

or a pharmaceutically acceptable salt, stereoisomer, solvate, prodrug, isotopic derivative, or tautomer thereof.

In some embodiments, the compound is of Formula (I-A-I-B-a-24′) or (I-A-I-B-a-24″):

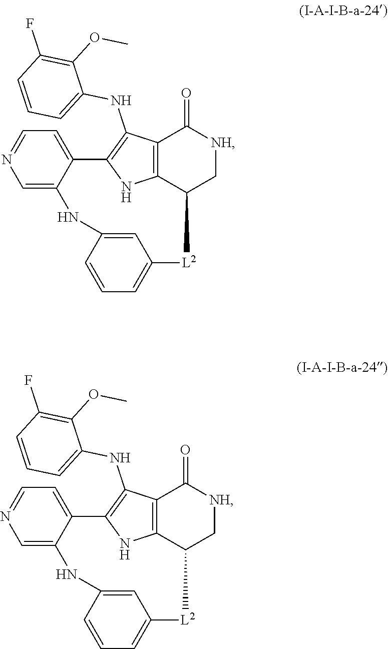

or a pharmaceutically acceptable salt, stereoisomer, solvate, prodrug, isotopic derivative, or tautomer thereof.

In some embodiments, the compound is of Formula (I-A-I-B-b-1):

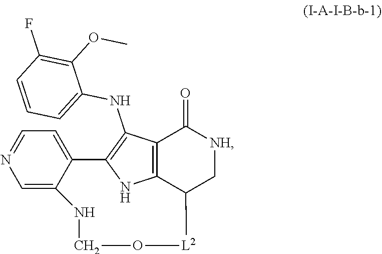

or a pharmaceutically acceptable salt, stereoisomer, solvate, prodrug, isotopic derivative, or tautomer thereof.

In some embodiments, the compound is of Formula (I-A-I-B-b-1′) or (I-A-I-B-b-1″):

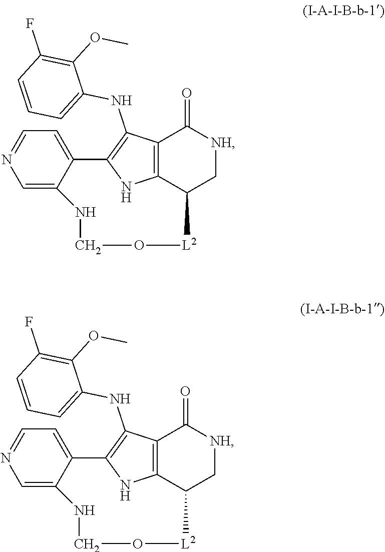

or a pharmaceutically acceptable salt, stereoisomer, solvate, prodrug, isotopic derivative, or tautomer thereof.

In some embodiments, the compound is of Formula (I-A-I-B-b-2):

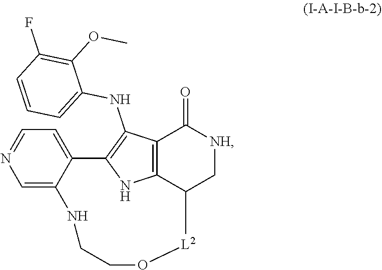

or a pharmaceutically acceptable salt, stereoisomer, solvate, prodrug, isotopic derivative, or tautomer thereof.

In some embodiments, the compound is of Formula (I-A-I-B-b-2′) or (I-A-I-B-b-2″):

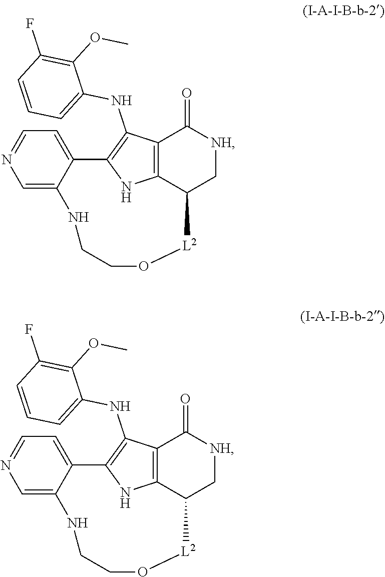

or a pharmaceutically acceptable salt, stereoisomer, solvate, prodrug, isotopic derivative, or tautomer thereof.

In some embodiments, the compound is of Formula (I-A-I-B-b-3):

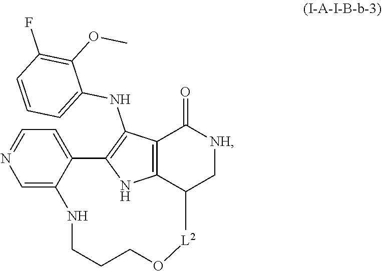

or a pharmaceutically acceptable salt, stereoisomer, solvate, prodrug, isotopic derivative, or tautomer thereof.

In some embodiments, the compound is of Formula (I-A-I-B-a-3′) or (I-A-I-B-a-3″):

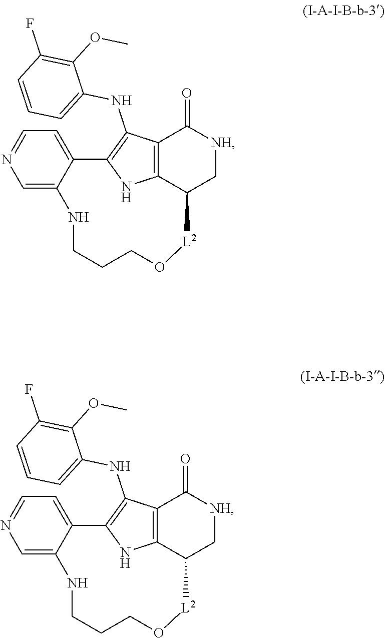

or a pharmaceutically acceptable salt, stereoisomer, solvate, prodrug, isotopic derivative, or tautomer thereof.

In some embodiments, the compound is of Formula (I-A-I-B-b-4):

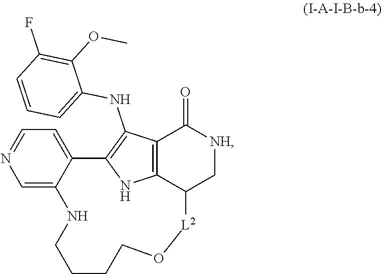

or a pharmaceutically acceptable salt, stereoisomer, solvate, prodrug, isotopic derivative, or tautomer thereof.

In some embodiments, the compound is of Formula (I-A-I-B-b-4′) or (I-A-I-B-b-4″):

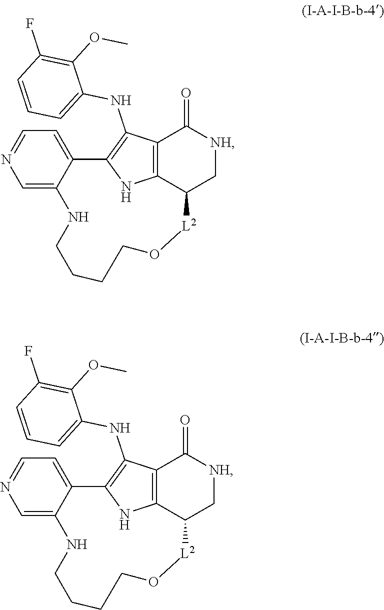

or a pharmaceutically acceptable salt, stereoisomer, solvate, prodrug, isotopic derivative, or tautomer thereof.

In some embodiments, the compound is of Formula (I-A-I-B-b-5):

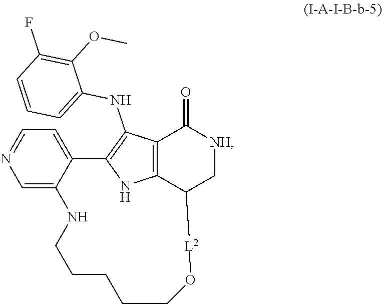

or a pharmaceutically acceptable salt, stereoisomer, solvate, prodrug, isotopic derivative, or tautomer thereof.

In some embodiments, the compound is of Formula (I-A-I-B-b-5′) or (I-A-I-B-b-5″):

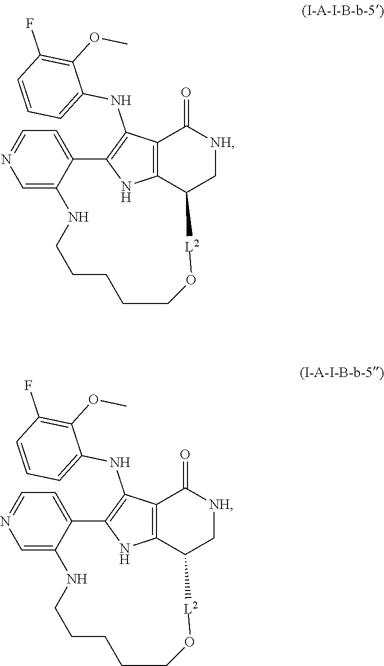

or a pharmaceutically acceptable salt, stereoisomer, solvate, prodrug, isotopic derivative, or tautomer thereof.

In some embodiments, the compound is of Formula (I-A-I-B-b-6):

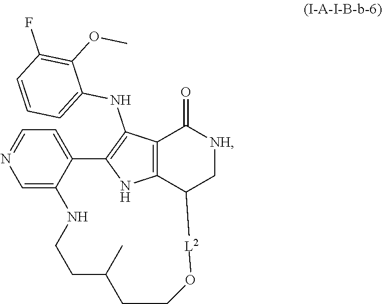

or a pharmaceutically acceptable salt, stereoisomer, solvate, prodrug, isotopic derivative, or tautomer thereof.

In some embodiments, the compound is of Formula (I-A-I-B-a-6′) or (I-A-I-B-a-6″):

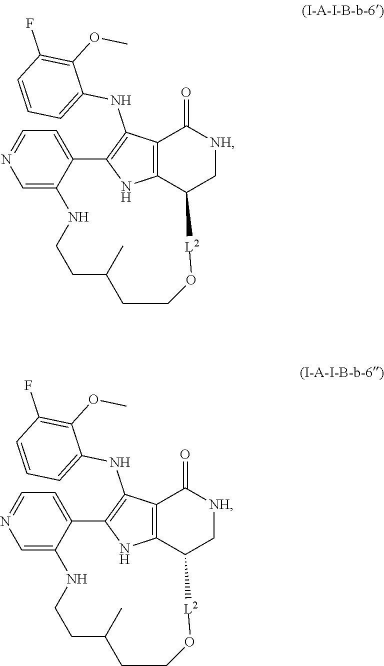

or a pharmaceutically acceptable salt, stereoisomer, solvate, prodrug, isotopic derivative, or tautomer thereof.

In some embodiments, the compound is of Formula (I-A-I-B-b-7):

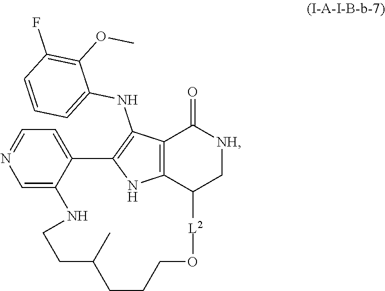

or a pharmaceutically acceptable salt, stereoisomer, solvate, prodrug, isotopic derivative, or tautomer thereof.

In some embodiments, the compound is of Formula (I-A-I-B-b-7′) or (I-A-I-B-b-7″):

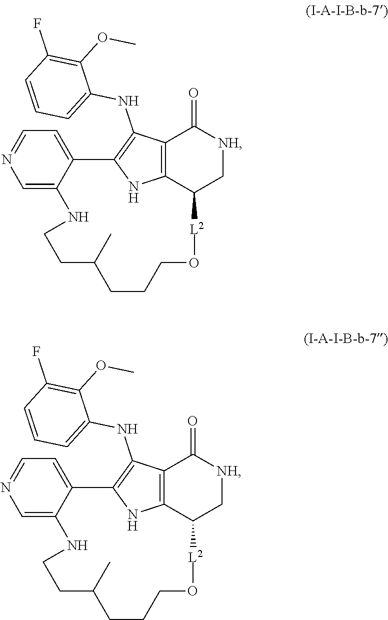

or a pharmaceutically acceptable salt, stereoisomer, solvate, prodrug, isotopic derivative, or tautomer thereof.

In some embodiments, the compound is of Formula (I-A-I-B-b-8):

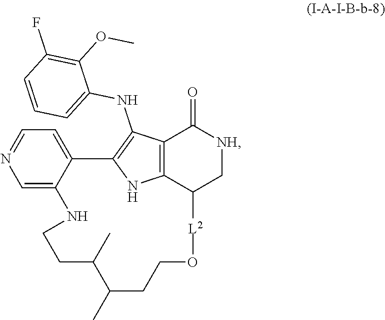

or a pharmaceutically acceptable salt, stereoisomer, solvate, prodrug, isotopic derivative, or tautomer thereof.

In some embodiments, the compound is of Formula (I-A-I-B-b-8′) or (I-A-I-B-b-8″):

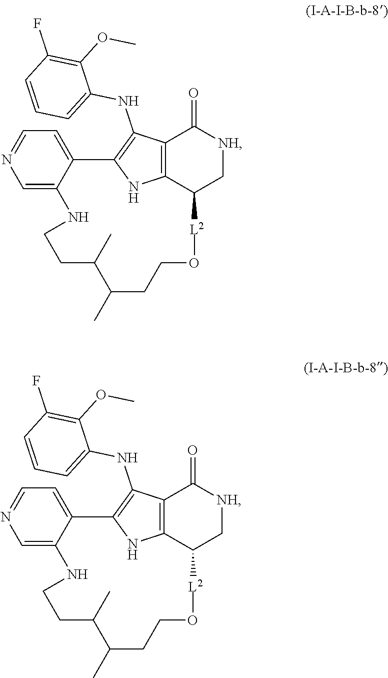

or a pharmaceutically acceptable salt, stereoisomer, solvate, prodrug, isotopic derivative, or tautomer thereof.

In some embodiments, the compound is of Formula (I-A-I-B-b-9):

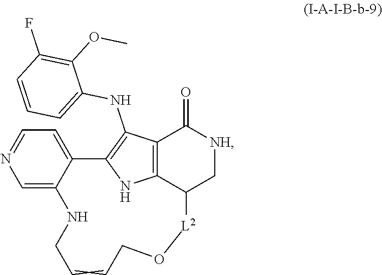

or a pharmaceutically acceptable salt, stereoisomer, solvate, prodrug, isotopic derivative, or tautomer thereof.

In some embodiments, the compound is of Formula (E-I-A-I-B-b-9) or (Z-I-A-I-B-b-9):

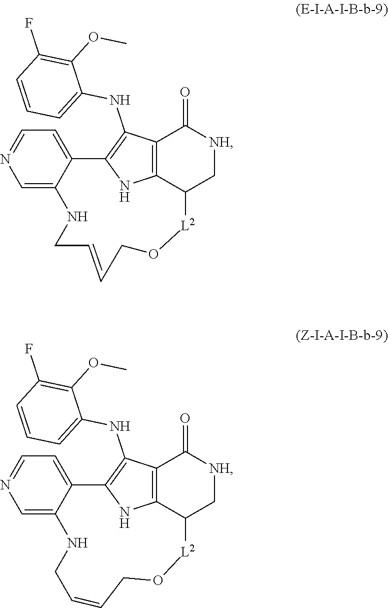

or a pharmaceutically acceptable salt, stereoisomer, solvate, prodrug, isotopic derivative, or tautomer thereof.

In some embodiments, the compound is of Formula (E-I-A-I-B-b-9′), (E-I-A-I-B-b-9″), (Z-I-A-T-B-b-9′) (Z-T-A-T-B-b-9″)

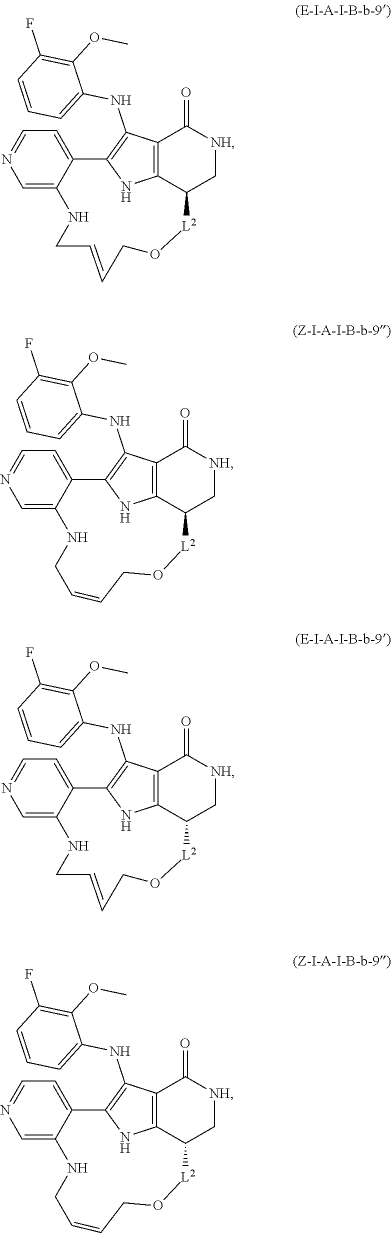

or a pharmaceutically acceptable salt, stereoisomer, solvate, prodrug, isotopic derivative, or tautomer thereof.

In some embodiments, the compound is of Formula (I-A-I-B-b-10):

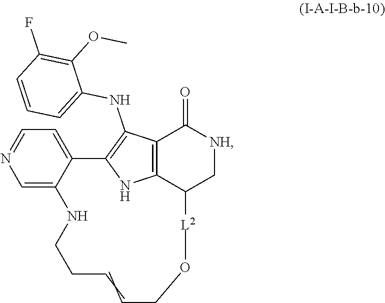

or a pharmaceutically acceptable salt, stereoisomer, solvate, prodrug, isotopic derivative, or tautomer thereof.

In some embodiments, the compound is of Formula (E-I-A-I-B-b-10) or (Z-I-A-I-B-b-10):

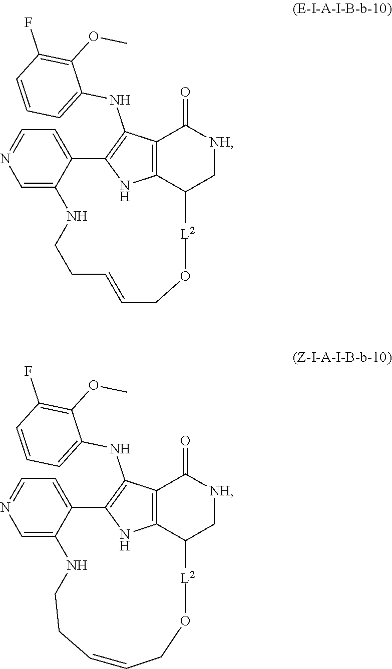

or a pharmaceutically acceptable salt, stereoisomer, solvate, prodrug, isotopic derivative, or tautomer thereof.

In some embodiments, the compound is of Formula (E-I-A-I-B-b-10′), (E-I-A-I-B-b-10″), (Z-I-A-I-B-b-10′), or (Z-I-A-I-B-b-10″):

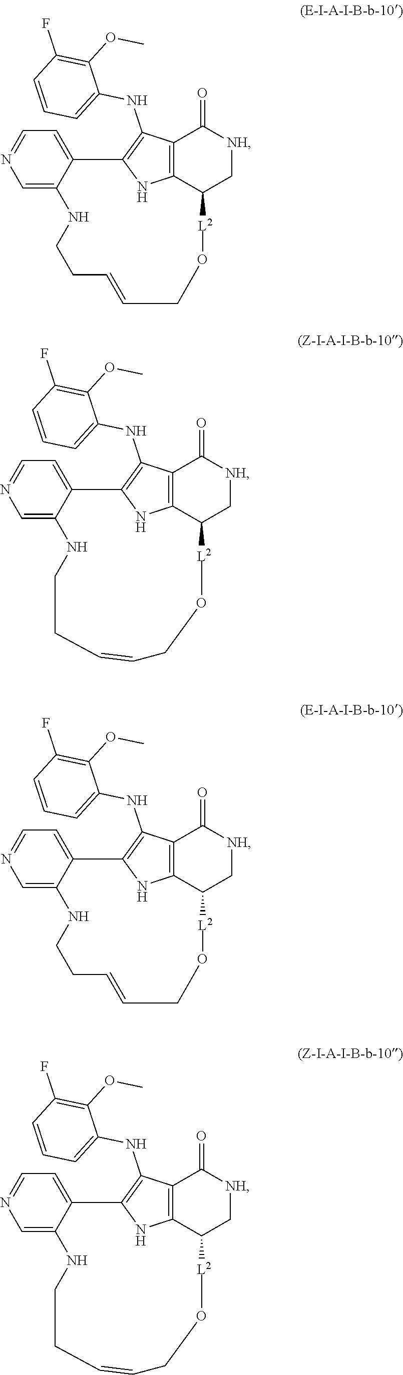

or a pharmaceutically acceptable salt, stereoisomer, solvate, prodrug, isotopic derivative, or tautomer thereof.

In some embodiments, the compound is of Formula (I-A-I-B-b-11):

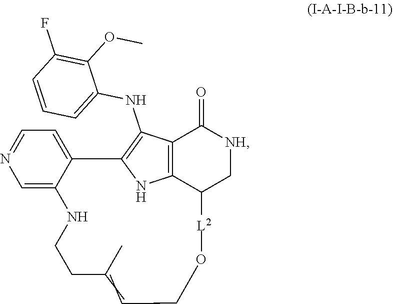

or a pharmaceutically acceptable salt, stereoisomer, solvate, prodrug, isotopic derivative, or tautomer thereof.

In some embodiments, the compound is of Formula (E-I-A-I-B-b-11) or (Z-I-A-I-B-b-11):

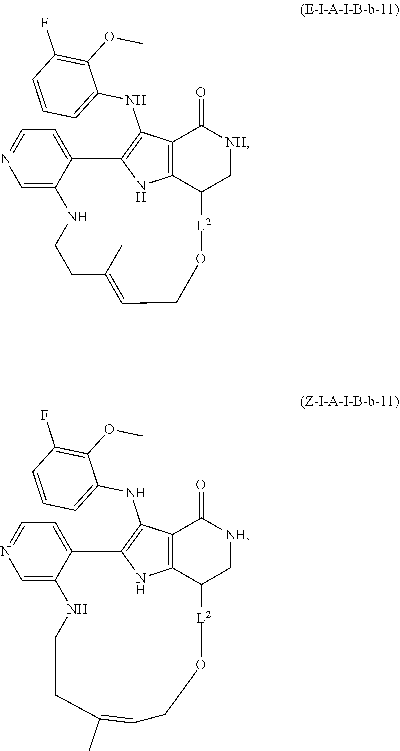

or a pharmaceutically acceptable salt, stereoisomer, solvate, prodrug, isotopic derivative, or tautomer thereof.

In some embodiments, the compound is of Formula (I-A-I-B-b-11′), (I-A-I-B-b-11″), (I-A-I-B-b-I1′), or (I-A-I-B-b-i1″):

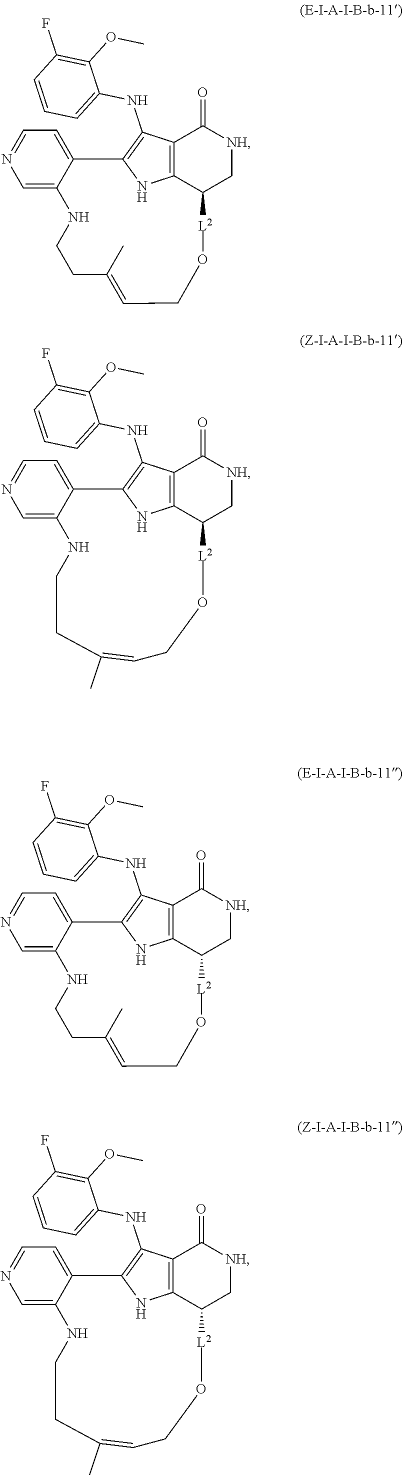

or a pharmaceutically acceptable salt, stereoisomer, solvate, prodrug, isotopic derivative, or tautomer thereof.

In some embodiments, the compound is of Formula (I-A-I-B-b-12):

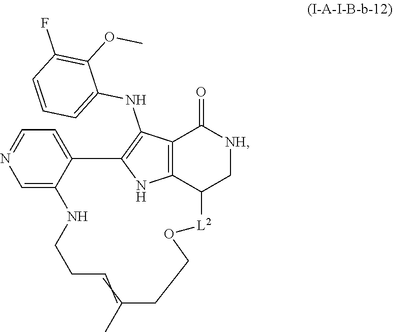

or a pharmaceutically acceptable salt, stereoisomer, solvate, prodrug, isotopic derivative, or tautomer thereof.

In some embodiments, the compound is of Formula (E-I-A-I-B-b-12) or (Z-I-A-I-B-b-12):

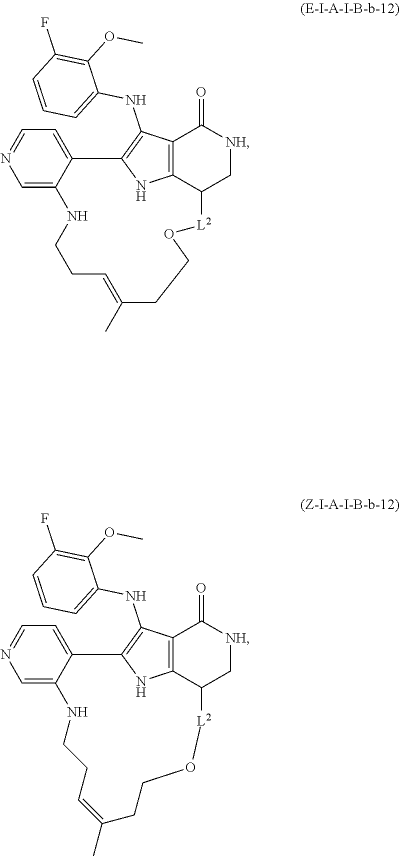

or a pharmaceutically acceptable salt, stereoisomer, solvate, prodrug, isotopic derivative, or tautomer thereof.

In some embodiments, the compound is of Formula (E-I-A-I-B-b-12′), (E-I-A-I-B-b-12″), (Z-I-A-I-B-b-12′), (Z-I-A-I-B-b-12″):

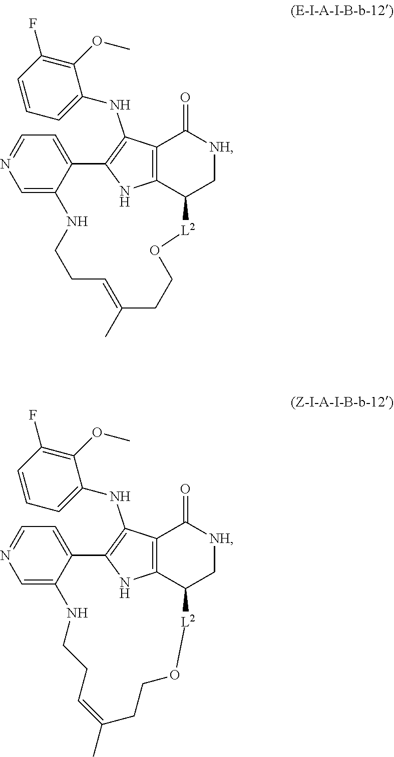

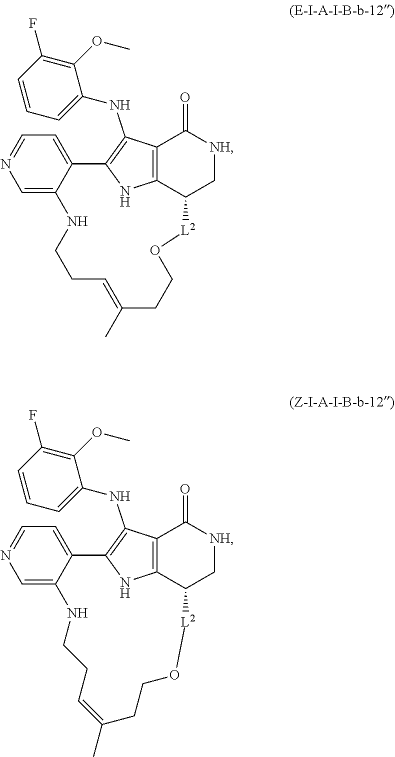

or a pharmaceutically acceptable salt, stereoisomer, solvate, prodrug, isotopic derivative, or tautomer thereof.

In some embodiments, the compound is of Formula (I-A-I-B-b-13):

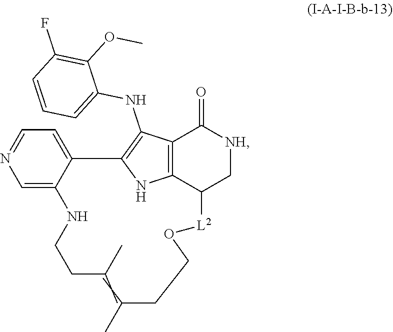

or a pharmaceutically acceptable salt, stereoisomer, solvate, prodrug, isotopic derivative, or tautomer thereof.

In some embodiments, the compound is of Formula (E-I-A-I-B-b-13) or (Z-I-A-I-B-b-13):

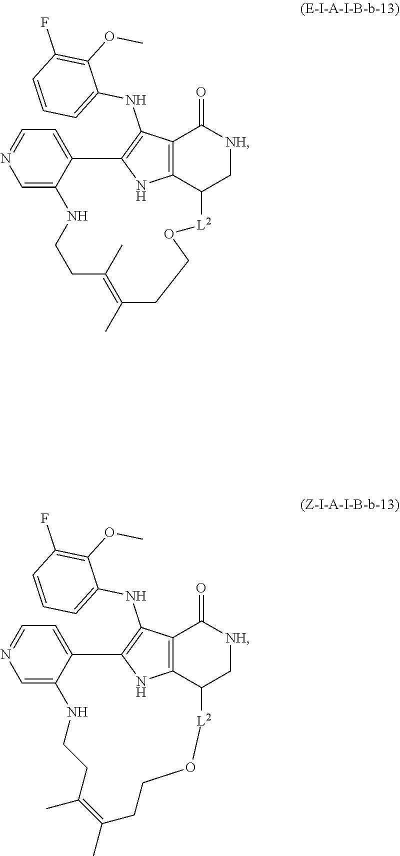

or a pharmaceutically acceptable salt, stereoisomer, solvate, prodrug, isotopic derivative, or tautomer thereof.

In some embodiments, the compound is of Formula (E-I-A-I-B-b-13′), (E-I-A-I-B-b-13″), (Z-I-A-I-B-b-13′), (Z-I-A-I-B-b-13″):

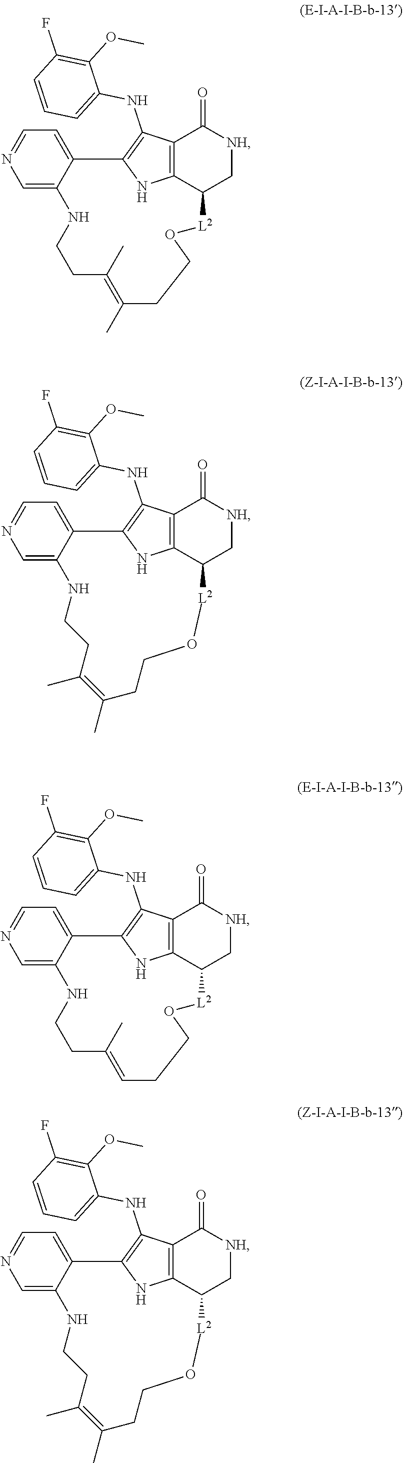

or a pharmaceutically acceptable salt, stereoisomer, solvate, prodrug, isotopic derivative, or tautomer thereof.

In some embodiments, the compound is of Formula (I-A-I-B-b-14):

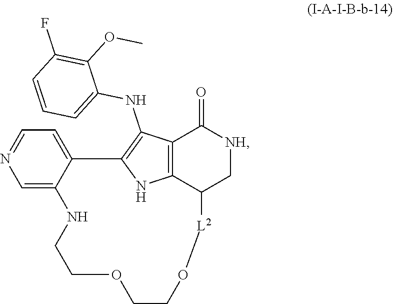

or a pharmaceutically acceptable salt, stereoisomer, solvate, prodrug, isotopic derivative, or tautomer thereof.

In some embodiments, the compound is of Formula (I-A-I-B-b-14′) or (I-A-I-B-b-14″):

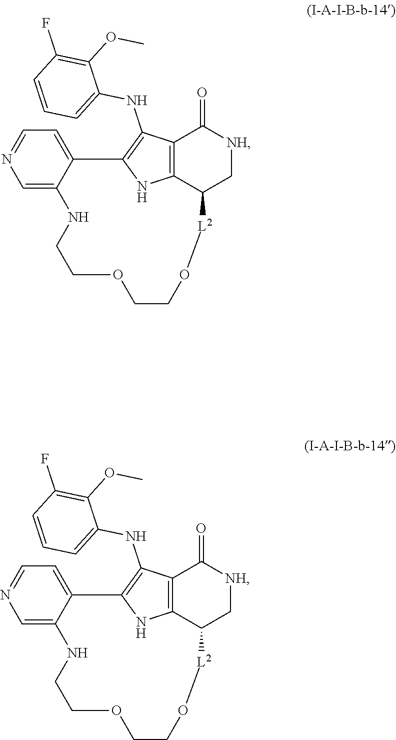

or a pharmaceutically acceptable salt, stereoisomer, solvate, prodrug, isotopic derivative, or tautomer thereof.

In some embodiments, the compound is of Formula (I-A-I-B-b-15):

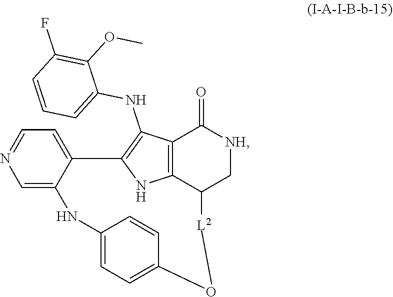

or a pharmaceutically acceptable salt, stereoisomer, solvate, prodrug, isotopic derivative, or tautomer thereof.

In some embodiments, the compound is of Formula (I-A-I-B-b-15′) or (I-A-I-B-b-15″):

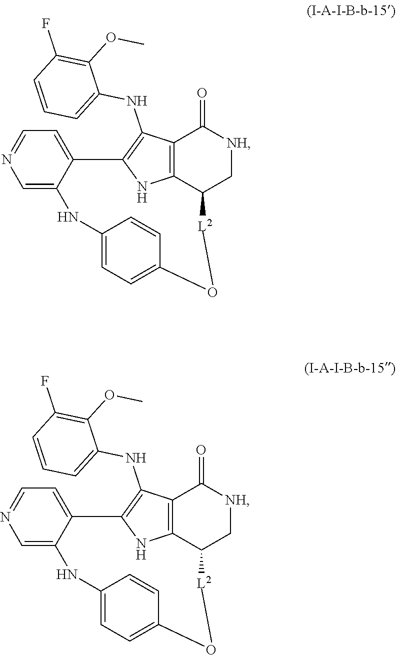

or a pharmaceutically acceptable salt, stereoisomer, solvate, prodrug, isotopic derivative, or tautomer thereof.

In some embodiments, the compound is of Formula (I-A-I-B-b-16):

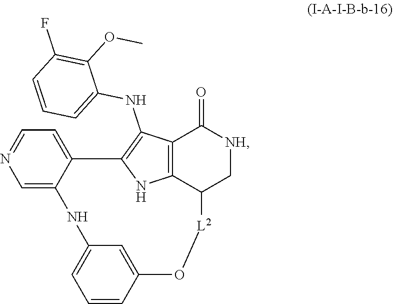

or a pharmaceutically acceptable salt, stereoisomer, solvate, prodrug, isotopic derivative, or tautomer thereof.

In some embodiments, the compound is of Formula (I-A-I-B-b-16′) or (I-A-I-B-b-16″):

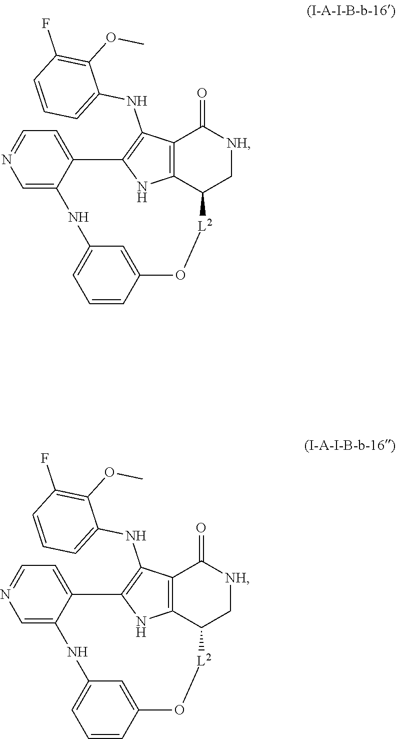

or a pharmaceutically acceptable salt, stereoisomer, solvate, prodrug, isotopic derivative, or tautomer thereof.

In some embodiments, the compound is of Formula (I-A-I-B-b-17):

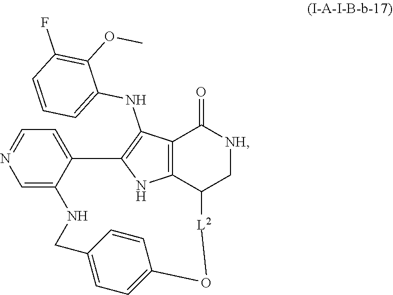

or a pharmaceutically acceptable salt, stereoisomer, solvate, prodrug, isotopic derivative, or tautomer thereof.

In some embodiments, the compound is of Formula (I-A-I-B-b-17′) or (I-A-I-B-b-17″):

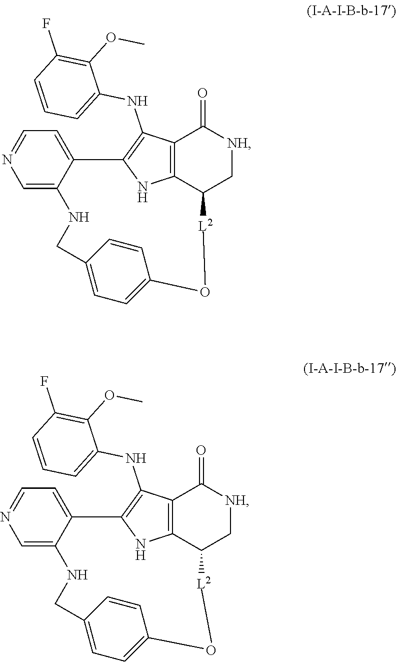

or a pharmaceutically acceptable salt, stereoisomer, solvate, prodrug, isotopic derivative, or tautomer thereof.

In some embodiments, the compound is of Formula (I-A-I-B-b-18):

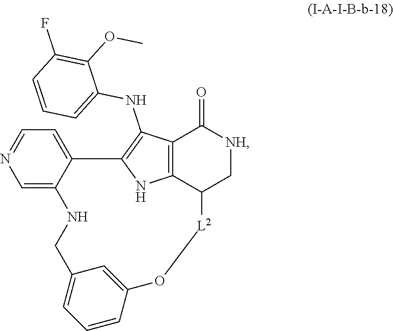

or a pharmaceutically acceptable salt, stereoisomer, solvate, prodrug, isotopic derivative, or tautomer thereof.

In some embodiments, the compound is of Formula (I-A-I-B-b-18′) or (I-A-I-B-b-18″):

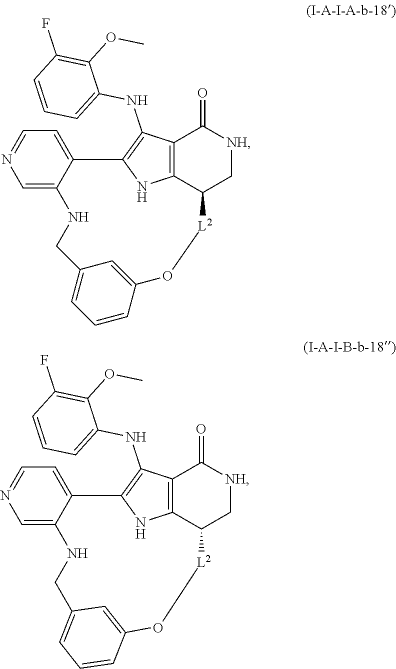

or a pharmaceutically acceptable salt, stereoisomer, solvate, prodrug, isotopic derivative, or tautomer thereof.

In some embodiments, the compound is of Formula (I-A-I-B-b-19):

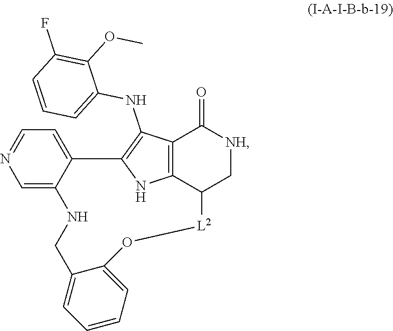

or a pharmaceutically acceptable salt, stereoisomer, solvate, prodrug, isotopic derivative, or tautomer thereof.

In some embodiments, the compound is of Formula (I-A-I-B-b-19′) or (I-A-I-B-b-19″):

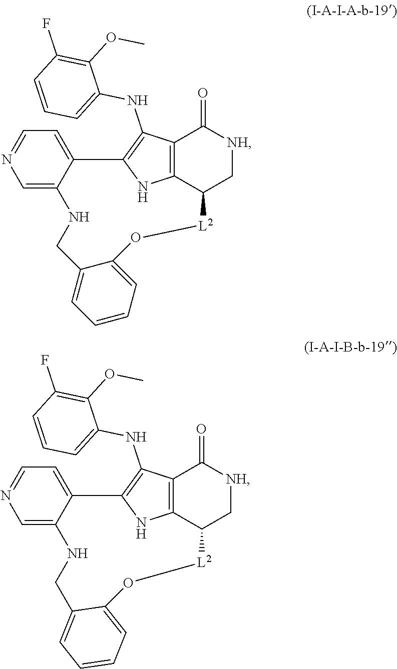

or a pharmaceutically acceptable salt, stereoisomer, solvate, prodrug, isotopic derivative, or tautomer thereof.

In some embodiments, the compound is of Formula (I-A-I-B-c-1):

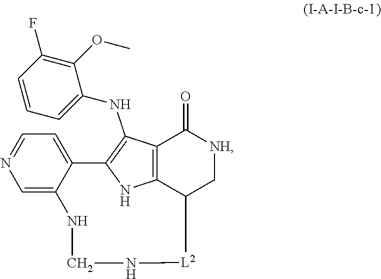

or a pharmaceutically acceptable salt, stereoisomer, solvate, prodrug, isotopic derivative, or tautomer thereof.

In some embodiments, the compound is of Formula (I-A-I-B-c-1′) or (I-A-I-B-c-1″):

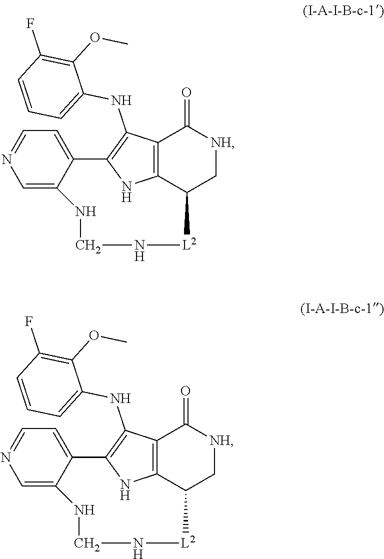

or a pharmaceutically acceptable salt, stereoisomer, solvate, prodrug, isotopic derivative, or tautomer thereof.

In some embodiments, the compound is of Formula (I-A-I-B-c-2):

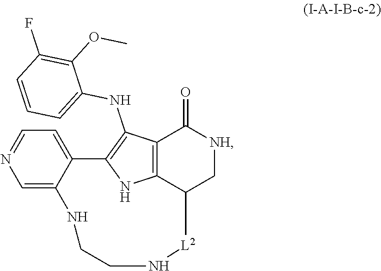

or a pharmaceutically acceptable salt, stereoisomer, solvate, pro rug, isotopic derivative, or tautomer thereof.

In some embodiments, the compound is of Formula (I-A-I-B-c-2′) or (I-A-I-B-c-2″):

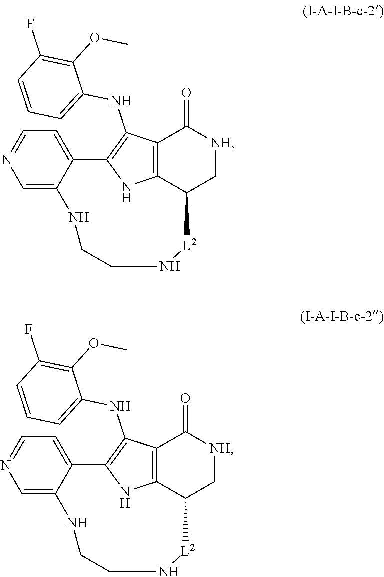

or a pharmaceutically acceptable salt, stereoisomer, solvate, prodrug, isotopic derivative, or tautomer thereof.

In some embodiments, the compound is of Formula (I-A-I-B-c-3):

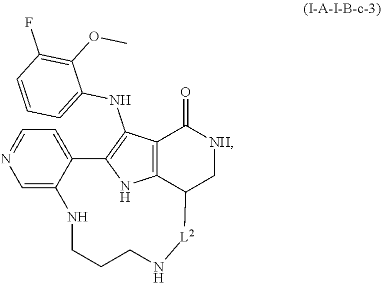

or a pharmaceutically acceptable salt, stereoisomer, solvate, prodrug, isotopic derivative, or tautomer thereof

In some embodiments, the compound is of Formula (I-A-I-B-c-3′) or (I-A-I-B-c-3″):

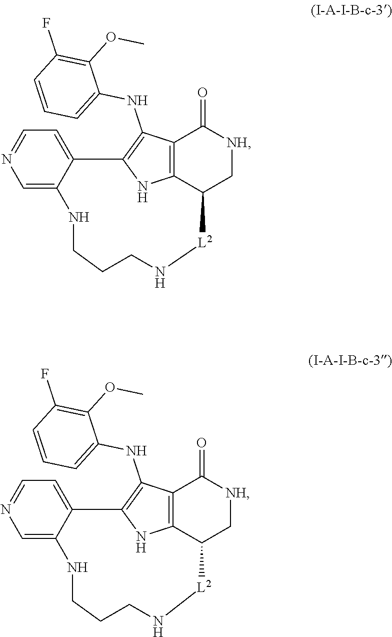

or a pharmaceutically acceptable salt, stereoisomer, solvate, prodrug, isotopic derivative, or tautomer thereof.

In some embodiments, the compound is of Formula (I-A-I-B-c-4):

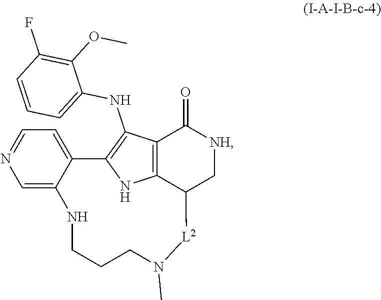

or a pharmaceutically acceptable salt, stereoisomer, solvate, prodrug, isotopic derivative, or tautomer thereof.

In some embodiments, the compound is of Formula (I-A-I-B-c-4′) or (I-A-I-B-c-4″):

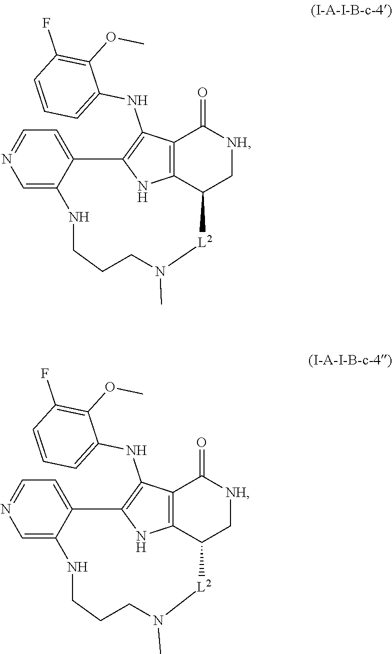

or a pharmaceutically acceptable salt, stereoisomer, solvate, prodrug, isotopic derivative, or tautomer thereof.

In some embodiments, the compound is of Formula (I-A-I-B-c-5):

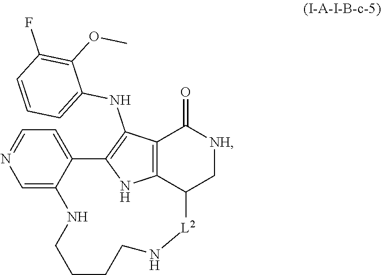

or a pharmaceutically acceptable salt, stereoisomer, solvate, prodrug, isotopic derivative, or tautomer thereof.

In some embodiments, the compound is of Formula (I-A-I-B-c-5′) or (I-A-I-B-c-5″):

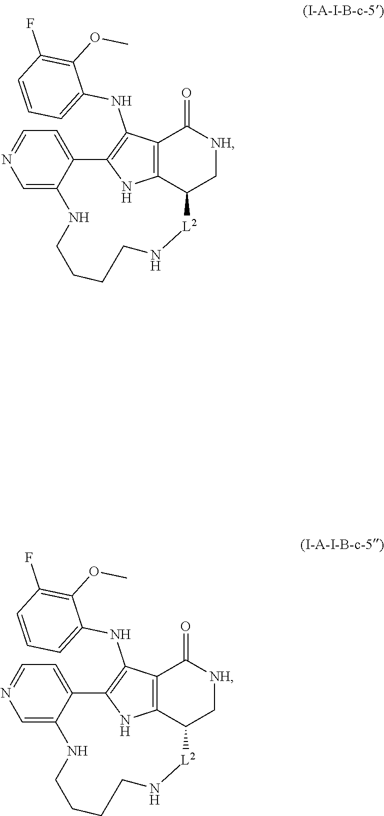

or a pharmaceutically acceptable salt, stereoisomer, solvate, prodrug, isotopic derivative, or tautomer thereof.

In some embodiments, the compound is of Formula (I-A-I-B-c-6):

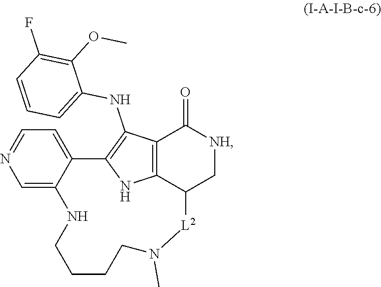

or a pharmaceutically acceptable salt, stereoisomer, solvate, prodrug, isotopic derivative, or tautomer thereof.

In some embodiments, the compound is of Formula (I-A-I-B-c-6′) or (I-A-I-B-c-6″):

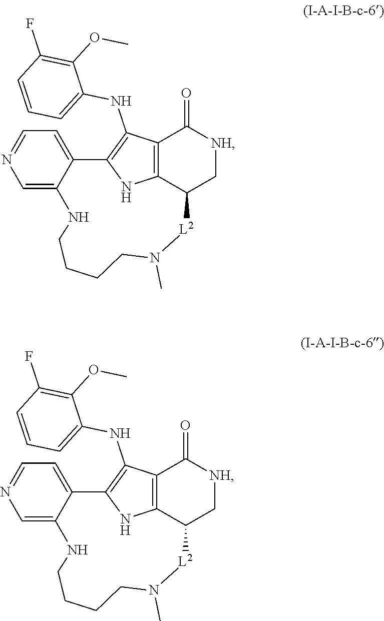

or a pharmaceutically acceptable salt, stereoisomer, solvate, prodrug, isotopic derivative, or tautomer thereof.

In some embodiments, the compound is of Formula (I-A-I-B-c-7):

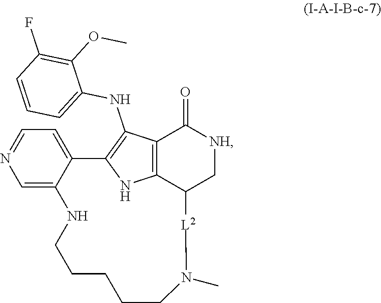

or a pharmaceutically acceptable salt, stereoisomer, solvate, prodrug, isotopic derivative, or tautomer thereof.

In some embodiments, the compound is of Formula (I-A-I-B-c-7′) or (I-A-I-B-c-7″):

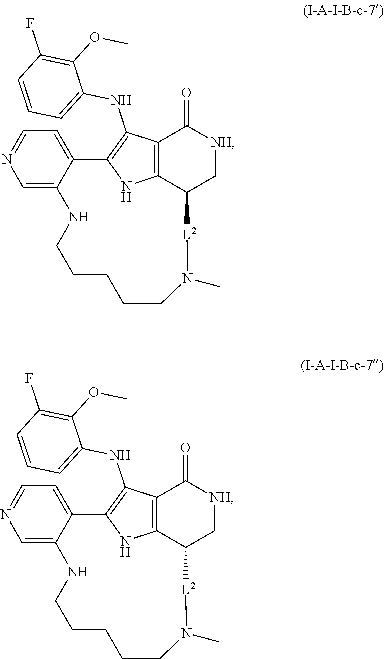

or a pharmaceutically acceptable salt, stereoisomer, solvate, prodrug, isotopic derivative, or tautomer thereof.

In some embodiments, the compound is of Formula (I-A-I-B-d-1):

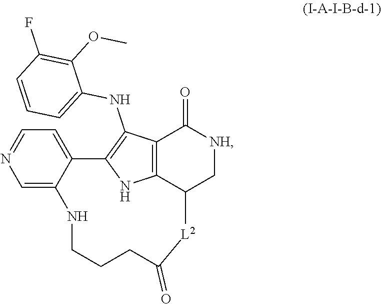

or a pharmaceutically acceptable salt, stereoisomer, solvate, prodrug, isotopic derivative, or tautomer thereof.

In some embodiments, the compound is of Formula (I-A-I-B-d-1′) or (I-A-I-B-d-1″):

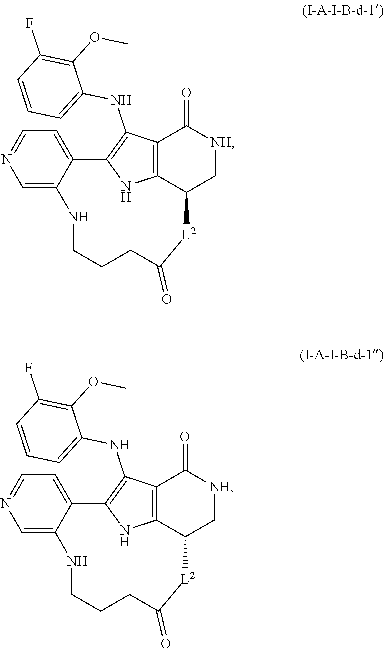

or a pharmaceutically acceptable salt, stereoisomer, solvate, prodrug, isotopic derivative, or tautomer thereof.

In some embodiments, the compound is of Formula (I-A-I-B-d-2):

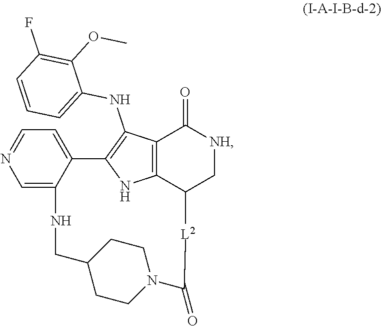

or a pharmaceutically acceptable salt, stereoisomer, solvate, prodrug, isotopic derivative, or tautomer thereof.

In some embodiments, the compound is of Formula (I-A-I-B-d-2′) or (I-A-I-B-d-2″):

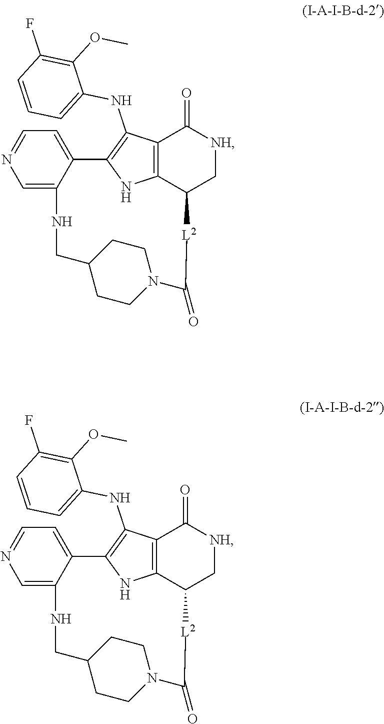

or a pharmaceutically acceptable salt, stereoisomer, solvate, prodrug, isotopic derivative, or tautomer thereof.

In some embodiments, the compound is of Formula (I-A-I-B-d-3):

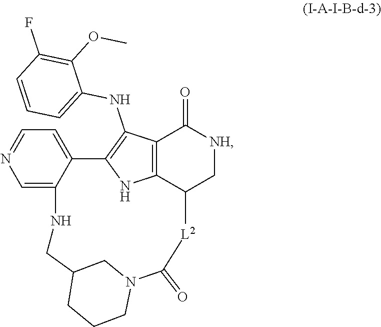

or a pharmaceutically acceptable salt, stereoisomer, solvate, prodrug, isotopic derivative, or tautomer thereof.

In some embodiments, the compound is of Formula (I-A-I-B-d-3′) or (I-A-I-B-d-3″):

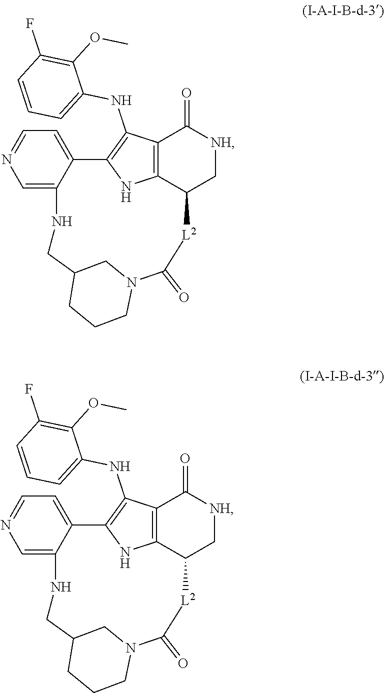

or a pharmaceutically acceptable salt, stereoisomer, solvate, prodrug, isotopic derivative, or tautomer thereof.

In some embodiments, the compound is of Formula (I-A-I-B-e-1):

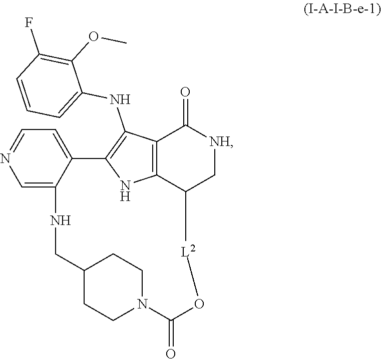

or a pharmaceutically acceptable salt, stereoisomer, solvate, prodrug, isotopic derivative, or tautomer thereof.

In some embodiments, the compound is of Formula (I-A-I-B-e-1′) or (I-A-I-B-e-1″):

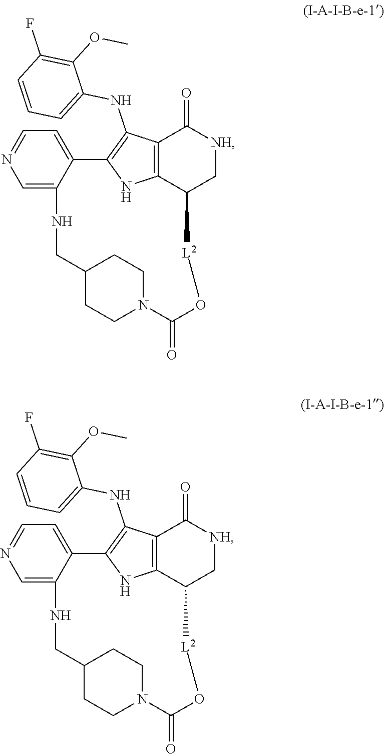

or a pharmaceutically acceptable salt, stereoisomer, solvate, prodrug, isotopic derivative, or tautomer thereof.

In some embodiments, the compound is of Formula (I-A-I-B-e-2):

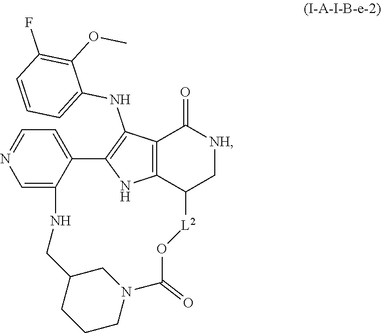

or a pharmaceutically acceptable salt, stereoisomer, solvate, prodrug, isotopic derivative, or tautomer thereof.

In some embodiments, the compound is of Formula (I-A-I-B-e-2′) or (I-A-I-B-e-2″):

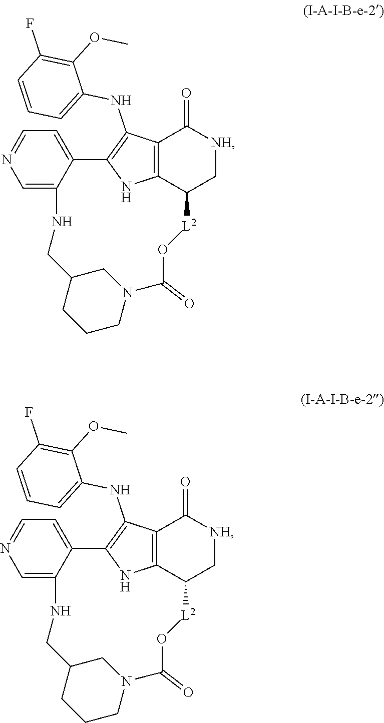

or a pharmaceutically acceptable salt, stereoisomer, solvate, prodrug, isotopic derivative, or tautomer thereof.

In some embodiments, the compound is of Formula (I-A-I-B-f-1):

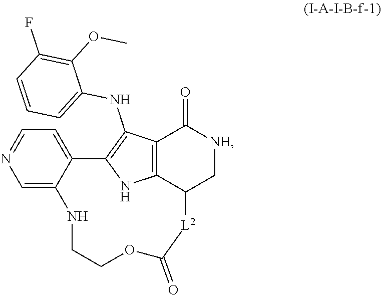

or a pharmaceutically acceptable salt, stereoisomer, solvate, prodrug, isotopic derivative, or tautomer thereof.

In some embodiments, the compound is of Formula (I-A-I-B-f-1′) or (I-A-I-B-f-2″):

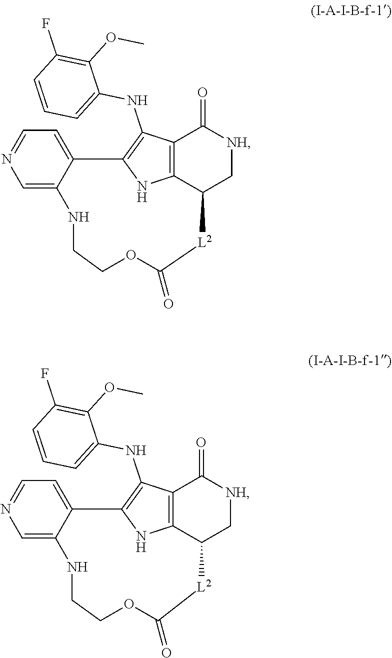

or a pharmaceutically acceptable salt, stereoisomer, solvate, prodrug, isotopic derivative, or tautomer thereof.

In some embodiments, the compound is of Formula (I-A-I-B-g-1):

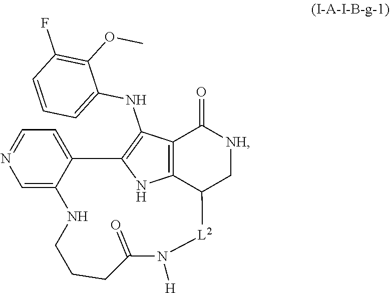

or a pharmaceutically acceptable salt, stereoisomer, solvate, prodrug, isotopic derivative, or tautomer thereof.

In some embodiments, the compound is of Formula (I-A-I-B-g-1′) or (I-A-I-B-g-1″):

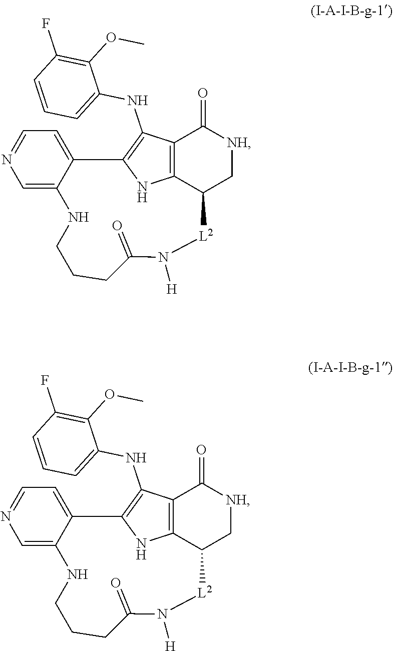

or a pharmaceutically acceptable salt, stereoisomer, solvate, prodrug, isotopic derivative, or tautomer thereof.

In some embodiments, the compound is of Formula (I-A-I-B-g-2):

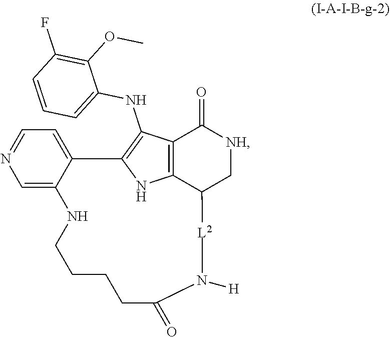

or a pharmaceutically acceptable salt, stereoisomer, solvate, prodrug, isotopic derivative, or tautomer thereof.

In some embodiments, the compound is of Formula (I-A-I-B-g-2′) or (I-A-I-B-g-2″):

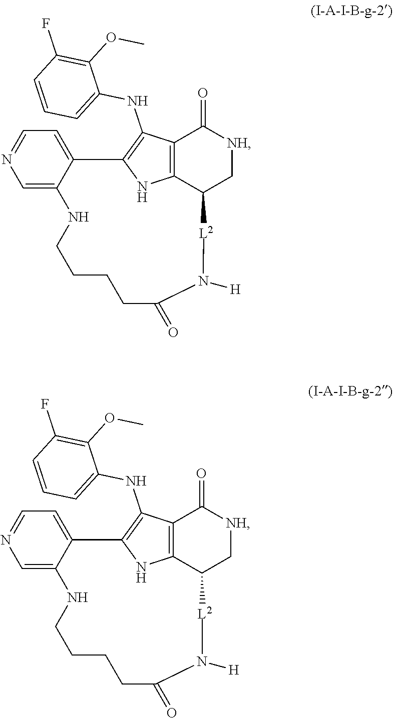

or a pharmaceutically acceptable salt, stereoisomer, solvate, prodrug, isotopic derivative, or tautomer thereof.

In some embodiments, the compound is of Formula (I-A-I-B-h-1):

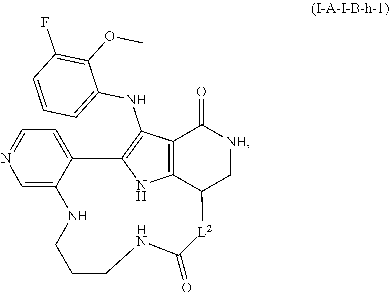

or a pharmaceutically acceptable salt, stereoisomer, solvate, prodrug, isotopic derivative, or tautomer thereof.

In some embodiments, the compound is of Formula (I-A-I-B-h-1′) or (I-A-I-B-h-1″):

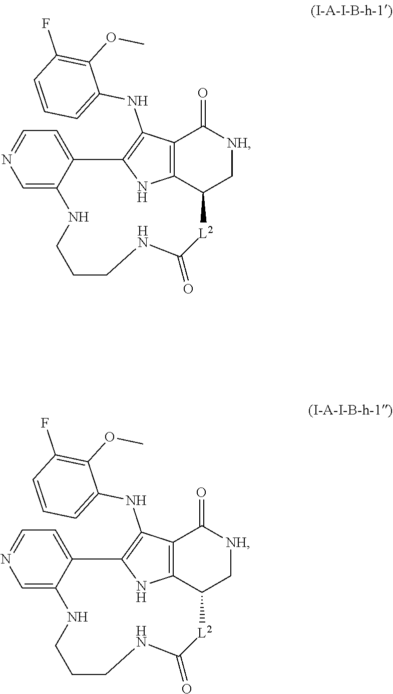

or a pharmaceutically acceptable salt, stereoisomer, solvate, prodrug, isotopic derivative, or tautomer thereof.

In some embodiments, the compound is of Formula (I-A-I-B-h-2):

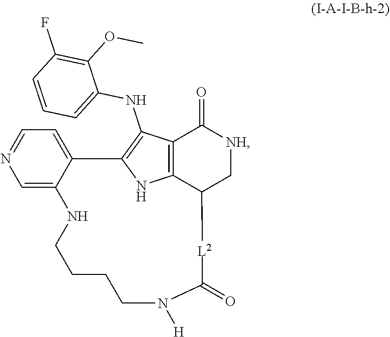

or a pharmaceutically acceptable salt, stereoisomer, solvate, prodrug, isotopic derivative, or tautomer thereof.

In some embodiments, the compound is of Formula (I-A-I-B-h-2′) or (I-A-I-B-h-2″):

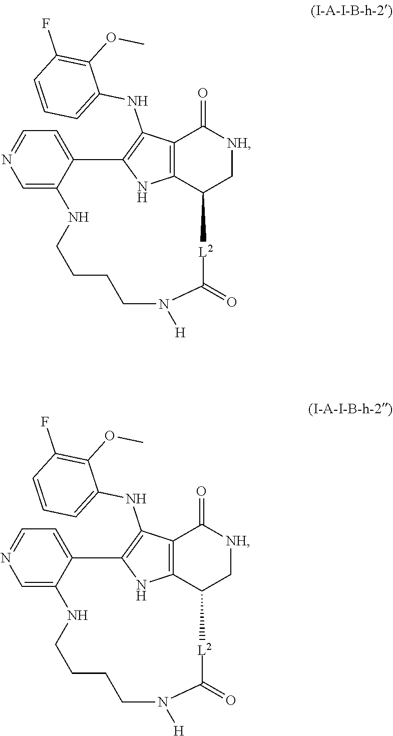

or a pharmaceutically acceptable salt, stereoisomer, solvate, prodrug, isotopic derivative, or tautomer thereof.

In some embodiments, the compound is of Formula (I-A-II):

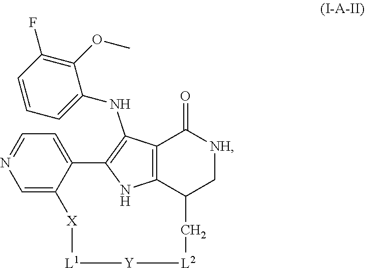

or a pharmaceutically acceptable salt, stereoisomer, solvate, prodrug, isotopic derivative, or tautomer thereof.

In some embodiments, the compound is of Formula (I-A-II-A):

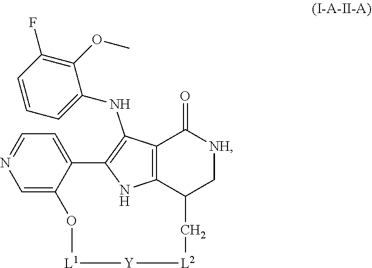

or a pharmaceutically acceptable salt, stereoisomer, solvate, prodrug, isotopic derivative, or tautomer thereof.

In some embodiments, the compound is of Formula (I-A-II-B):

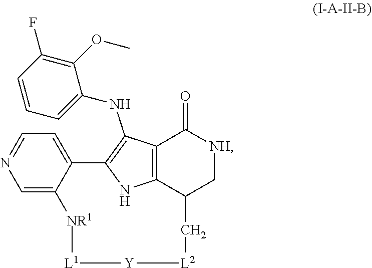

or a pharmaceutically acceptable salt, stereoisomer, solvate, prodrug, isotopic derivative, or tautomer thereof.

In some embodiments, the compound is of Formula (A-B):

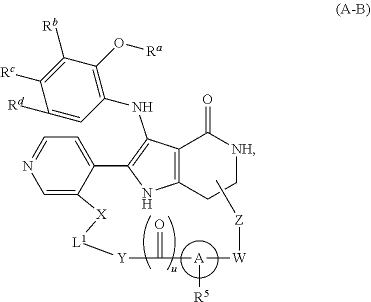

or a pharmaceutically acceptable salt, stereoisomer, solvate, or tautomer thereof, wherein
R^{a} is selected from C₁-C₆ alkyl, H, C₂-C₆ alkenyl, C₂-C₆ alkynyl, C₃-C₁₀ cycloalkyl, heterocyclyl, aryl, and heteroaryl, wherein the alkyl, alkenyl, alkynyl, cycloalkyl, heterocyclyl, aryl, or heteroaryl is optionally substituted with one or more substituents independently selected from halogen, OH, ═O, NR³R⁴, CN, NO₂, COOR¹, CONR³R⁴;
R^{b}, R^{c}, R^{d} are each independently selected from H, halogen, OH, NH₂, CN, NO₂, C₁-C₆ alkyl, and C₁-C₆ alkoxy, wherein alkyl or alkoxy is optionally substituted with one or more halogen, CN;
X is selected from a bond, O, NR¹, C(R²)₂, S, S(O), S(O)₂, C(O), C(O)O, C(O)NR¹, OC(O)O, N(R²)C(O)NR², arenediyl;
R¹ is selected from H, C₁-C₆ alkyl, C₃-C₁₀ cycloalkyl, C₂-C₆ alkenyl, C₂-C₆ alkynyl, C₁-C₆ acyl, heterocycle, aryl, heteroaryl, and S(O)₂R² wherein the alkyl, alkenyl, alkynyl, heterocycle, cycloalkyl, aryl, or heteroaryl is optionally substituted with one or more substituents independently selected from halogen, OH, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, heteroaryl;
each R² is independently selected from H, C₁-C₆ alkyl, C₃-C₁₀ cycloalkyl, C₂-C₆ alkenyl, C₂-C₆ alkynyl, heterocycle, aryl, and heteroaryl wherein the alkyl, alkenyl, alkynyl, heterocycle, cycloalkyl, aryl, or heteroaryl is optionally substituted with one or more substituents independently selected from halogen, OH, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, heteroaryl;
L¹ is selected from C₁-C₁₀ alkanediyl, C₂-C₁₀ alkenediyl, C₂-C₁₀ alkynediyl, C₃-C₁₀-cycloalkanediyl, C₃-C₁₀-cycloalkanediyl-C₁-C₁₀-alkanediyl, arenediyl, arenediyl-C₁-C₁₀-alkanediyl, heterocyclediyl, heterocyclediyl-C₁-C₁₀-alkanediyl, heterocyclediyl-C(O), heteroarenediyl, heteroarenediyl-C₁-C₁₀ alkanediyl, C₁-C₆ alkanediyl-O—C₁-C₆ alkanediyl, C₁-C₆ alkanediyl-NR¹—C₁-C₆ alkanediyl, C₁-C₆ alkanediyl-C(O)NR¹—C₁-C₆ alkanediyl, wherein alkanediyl, alkendiyl, alkynediyl, arenediyl, heterocyclediyl or heteroarenediyl heteroaryl is optionally substituted with one or more substituents independently selected from halogen, OH, oxo, CN, C₁-C₆ alkyl, C₁-C₆ alkoxy, C₁-C₆ alkyl-C₁-C₆ alkoxy, C₁-C₆ alkyl-NR¹—C₁-C₆ alkyl, NR³R⁴, —C(O)NR³R⁴, —S(O)C₁-C₆ alkyl, —S(O)₂NR³R⁴, aryl, and —S(O)₂C₁-C₆ alkyl;
Y is selected from a bond, O, NR¹, C(R²)₂, S, S(O), S(O)₂, C(O), C(O)O, C(O)NR¹, OC(O)O, N(R²)C(O)NR²;
Z is selected from a bond, O, C(R²)₂;
Ring A is selected from C₃-C₁₀-cycloalkane, arene, heterocycle, heteroarene;
W is (CH₂)_{w};
w is an integer selected from 0, 1, 2, 3, 4, 5, 6, 7, 8, 9, and 10;
u is an integer selected from 0 and 1;
R³ and R⁴ are each independently selected from H, C₁-C₆ alkyl, C₁-C₆ acyl and C₃-C₁₀ cycloalkyl;
or R³ and R⁴ together with the atoms to which they are attached and any intervening atoms, form a 5-14 membered heterocycle, wherein the heterocycle is optionally substituted with one or more substituents independently selected from halogen, OH, ═O, CN, NO₂, C₁-C₆ alkyl, C₁-C₆ alkoxy;
R⁵ is selected from H, halogen, OH, oxo, CN, C₁-C₆ alkyl, C₁-C₆ alkoxy, C₁-C₆ alkyl-C₁-C₆ alkoxy, C₁-C₆ alkyl-NR¹—C₁-C₆ alkyl, NR³R⁴, —C(O)NR³R⁴, —S(O)C₁-C₆ alkyl, —S(O)₂NR³R⁴, and —S(O)₂C₁-C₆ alkyl.

In some embodiments, the compound is of Formula (I-A-II-A-a):

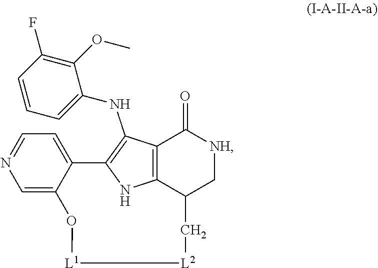

or a pharmaceutically acceptable salt, stereoisomer, solvate, prodrug, isotopic derivative, or tautomer thereof.

In some embodiments, the compound is of Formula (I-A-II-A-a-1):

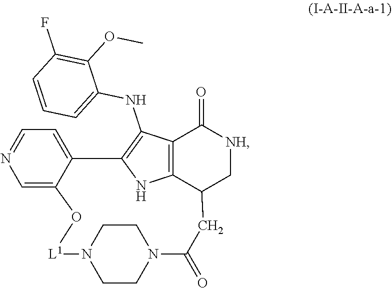

or a pharmaceutically acceptable salt, stereoisomer, solvate, prodrug, isotopic derivative, or tautomer thereof.

In some embodiments, the compound is of Formula (I-A-II-A-h):

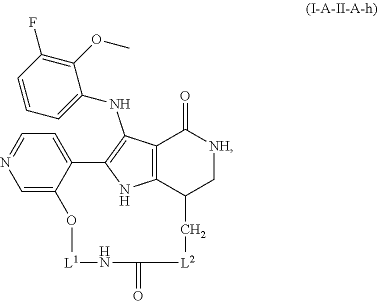

or a pharmaceutically acceptable salt, stereoisomer, solvate, prodrug, isotopic derivative, or tautomer thereof.

In some embodiments, the compound is of Formula (I-A-II-A-h-1):

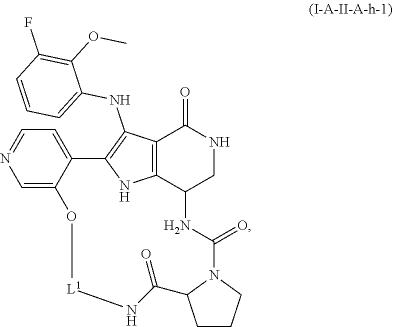

or a pharmaceutically acceptable salt, stereoisomer, solvate, prodrug, isotopic derivative, or tautomer thereof.

In some embodiments, the compound is of Formula (I-A-II-A-h-2):

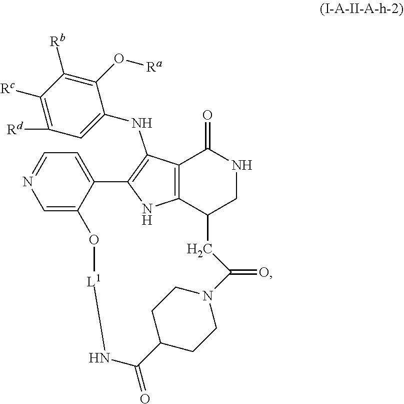

or a pharmaceutically acceptable salt, stereoisomer, solvate, pro rug, isotopic derivative, or tautomer thereof.

In some embodiments, the compound is of Formula (II′):

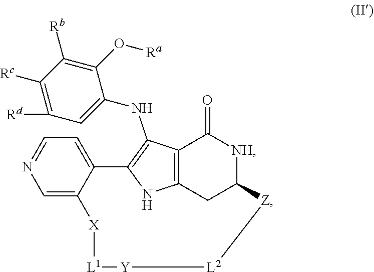

or a pharmaceutically acceptable salt, stereoisomer, solvate, prodrug, isotopic derivative, or tautomer thereof wherein all variables are as defined herein.

In some embodiments, the compound is of Formula (II″):

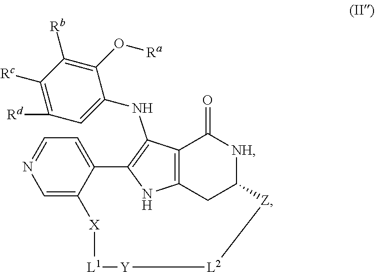

or a pharmaceutically acceptable salt, stereoisomer, solvate, prodrug, isotopic derivative, or tautomer thereof wherein all variables are as defined herein.

In some embodiments, the compound is of Formula (II-A′):

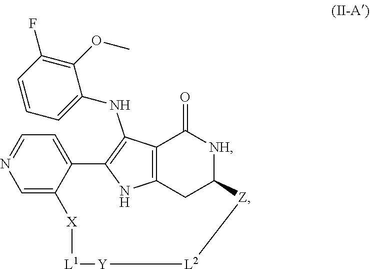

or a pharmaceutically acceptable salt, stereoisomer, solvate, prodrug, isotopic derivative, or tautomer thereof wherein all variables are as defined herein.

In some embodiments, the compound is of Formula (II-A″):

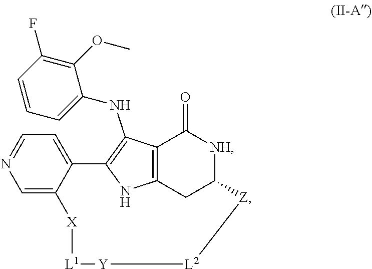

or a pharmaceutically acceptable salt, stereoisomer, solvate, prodrug, isotopic derivative, or tautomer thereof wherein all variables are as defined herein.

In some embodiments, the compound is of Formula (II-A-I):

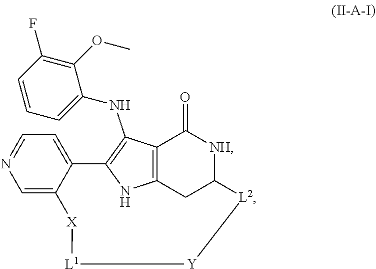

or a pharmaceutically acceptable salt, stereoisomer, solvate, prodrug, isotopic derivative, or tautomer thereof.

In some embodiments, the compound is of Formula (II-A-I′):

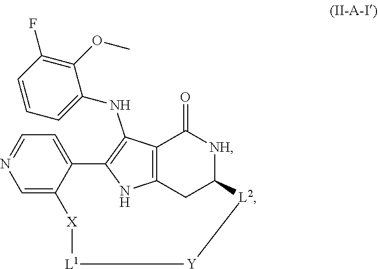

or a pharmaceutically acceptable salt, stereoisomer, solvate, prodrug, isotopic derivative, or tautomer thereof.

In some embodiments, the compound is of Formula (II-A-I″):

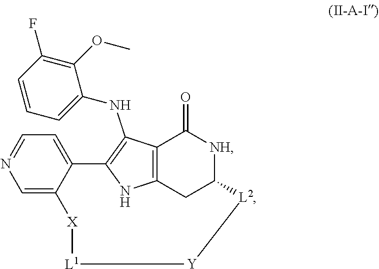

or a pharmaceutically acceptable salt, stereoisomer, solvate, prodrug, isotopic derivative, or tautomer thereof.

In some embodiments, the compound is of Formula (II-A-I-A):

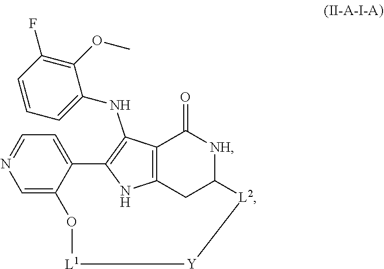

or a pharmaceutically acceptable salt, stereoisomer, solvate, prodrug, isotopic derivative, or tautomer thereof.

In some embodiments, the compound is of Formula (II-A-I-A′):

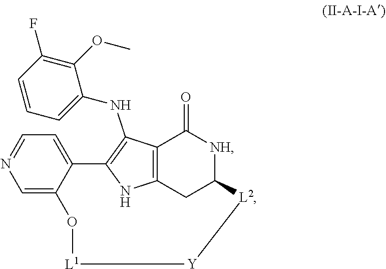

or a pharmaceutically acceptable salt, stereoisomer, solvate, prodrug, isotopic derivative, or tautomer thereof.

In some embodiments, the compound is of Formula (II-A-I-A″):

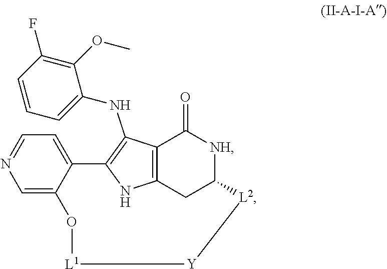

or a pharmaceutically acceptable salt, stereoisomer, solvate, prodrug, isotopic derivative, or tautomer thereof.

In some embodiments, the compound is of Formula (II-A-I-B):

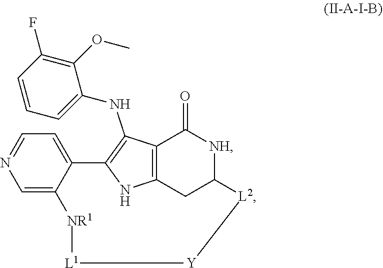

or a pharmaceutically acceptable salt, stereoisomer, solvate, prodrug, isotopic derivative, or tautomer thereof.

In some embodiments, the compound is of Formula (II-A-I-B′):

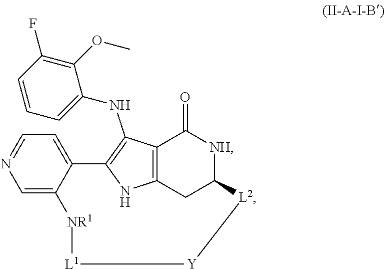

or a pharmaceutically acceptable salt, stereoisomer, solvate, prodrug, isotopic derivative, or tautomer thereof.

In some embodiments, the compound is of Formula (II-A-I-B″):

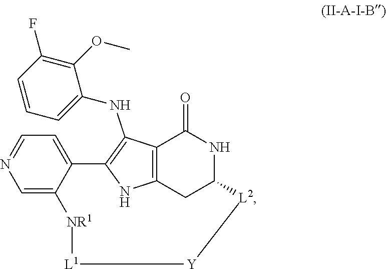

or a pharmaceutically acceptable salt, stereoisomer, solvate, prodrug, isotopic derivative, or tautomer thereof.

In some embodiments, the compound is of Formula (II-A-I-B-H):

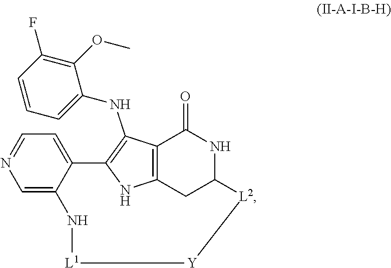

or a pharmaceutically acceptable salt, stereoisomer, solvate, prodrug, isotopic derivative, or tautomer thereof.

In some embodiments, the compound is of Formula (II-A-I-B-H′):

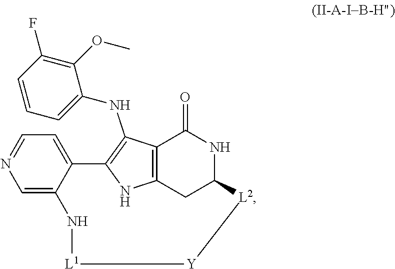

or a pharmaceutically acceptable salt, stereoisomer, solvate, prodrug, isotopic derivative, or tautomer thereof.

In some embodiments, the compound is of Formula (II-A-I-B-H″):

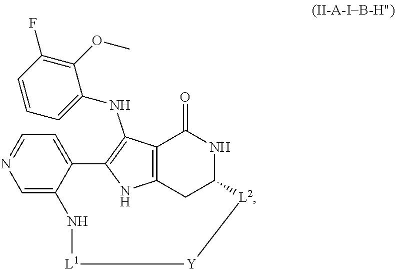

or a pharmaceutically acceptable salt, stereoisomer, solvate, prodrug, isotopic derivative, or tautomer thereof.

In some embodiments, the compound is of Formula (II-A-I-C):

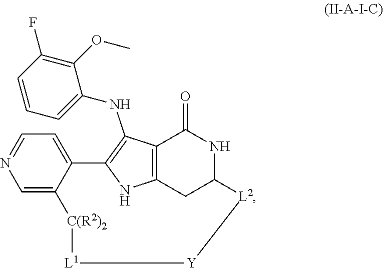

or a pharmaceutically acceptable salt, stereoisomer, solvate, prodrug, isotopic derivative, or tautomer thereof.

In some embodiments, the compound is of Formula (II-A-I-C′):

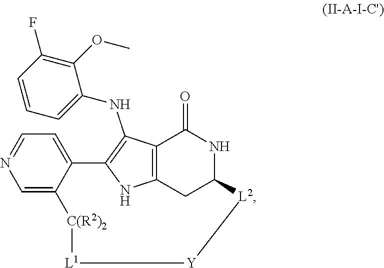

or a pharmaceutically acceptable salt, stereoisomer, solvate, prodrug, isotopic derivative, or tautomer thereof.

In some embodiments, the compound is of Formula (II-A-I-C″):

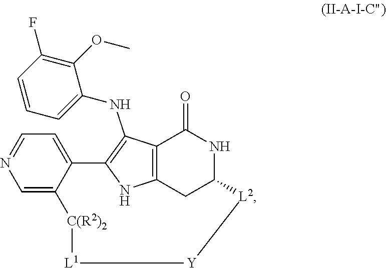

or a pharmaceutically acceptable salt, stereoisomer, solvate, prodrug, isotopic derivative, or tautomer thereof.

In some embodiments, the compound is of Formula (II-A-I-C-H):

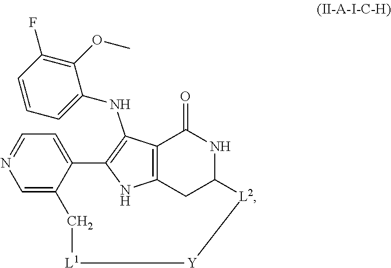

or a pharmaceutically acceptable salt, stereoisomer, solvate, prodrug, isotopic derivative, or tautomer thereof.

In some embodiments, the compound is of Formula (II-A-I-C-H′):

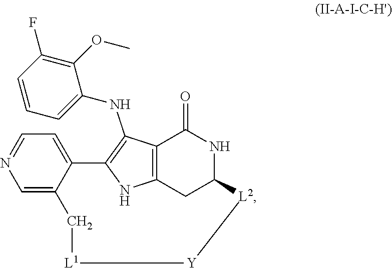

or a pharmaceutically acceptable salt, stereoisomer, solvate, prodrug, isotopic derivative, or tautomer thereof.

In some embodiments, the compound is of Formula (II-A-I-C-H″):

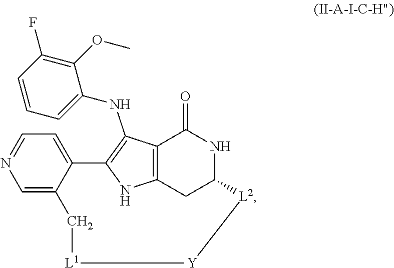

or a pharmaceutically acceptable salt, stereoisomer, solvate, prodrug, isotopic derivative, or tautomer thereof.

In some embodiments, the compound is of Formula (II-A-I-D):

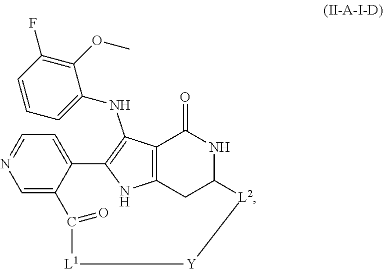

or a pharmaceutically acceptable salt, stereoisomer, solvate, prodrug, isotopic derivative, or tautomer thereof.

In some embodiments, the compound is of Formula (II-A-I-D′):

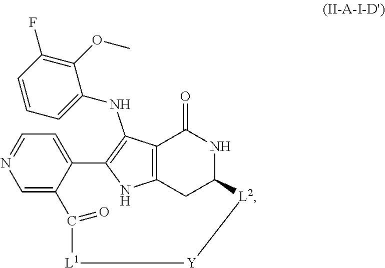

or a pharmaceutically acceptable salt, stereoisomer, solvate, prodrug, isotopic derivative, or tautomer thereof.

In some embodiments, the compound is of Formula (II-A-I-D″):

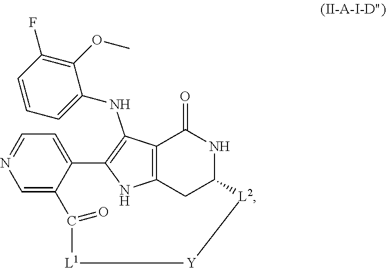

or a pharmaceutically acceptable salt, stereoisomer, solvate, prodrug, isotopic derivative, or tautomer thereof.

In some embodiments, the compound is of Formula (II-A-I-E):

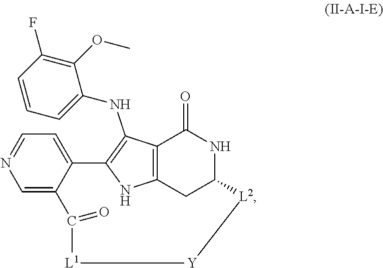

or a pharmaceutically acceptable salt, stereoisomer, solvate, prodrug, isotopic derivative, or tautomer thereof.

In some embodiments, the compound is of Formula (II-A-I-E′):

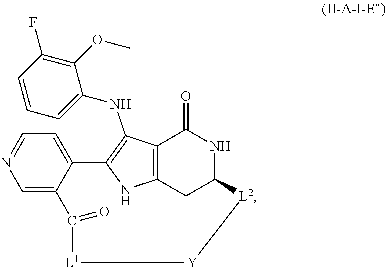

or a pharmaceutically acceptable salt, stereoisomer, solvate, prodrug, isotopic derivative, or tautomer thereof.

In some embodiments, the compound is of Formula (II-A-I-E″):

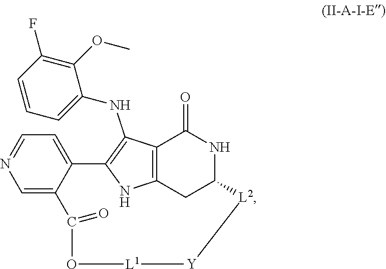

or a pharmaceutically acceptable salt, stereoisomer, solvate, prodrug, isotopic derivative, or tautomer thereof.

In some embodiments, the compound is of Formula (II-A-I-F):

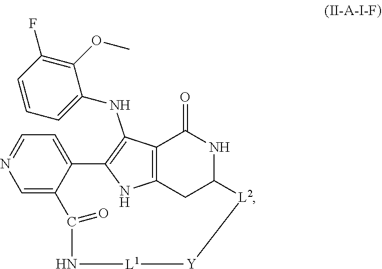

or a pharmaceutically acceptable salt, stereoisomer, solvate, prodrug, isotopic derivative, or tautomer thereof.

In some embodiments, the compound is of Formula (II-A-I-F′):

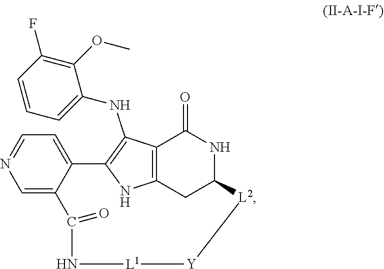

or a pharmaceutically acceptable salt, stereoisomer, solvate, prodrug, isotopic derivative, or tautomer thereof.

In some embodiments, the compound is of Formula (II-A-I-F″):

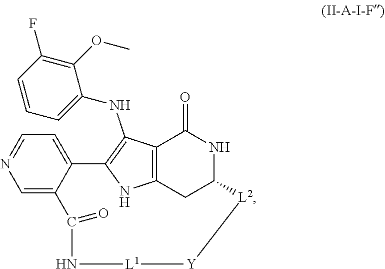

or a pharmaceutically acceptable salt, stereoisomer, solvate, prodrug, isotopic derivative, or tautomer thereof.

In some embodiments, the compound is of Formula (II-A-I-G):

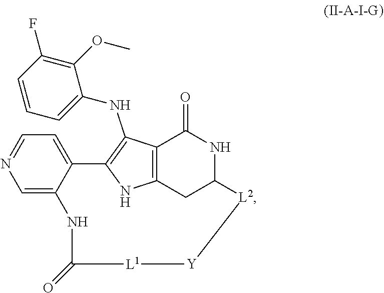

or a pharmaceutically acceptable salt, stereoisomer, solvate, prodrug, isotopic derivative, or tautomer thereof.

In some embodiments, the compound is of Formula (II-A-I-G′):

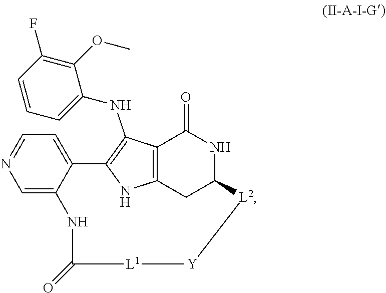

or a pharmaceutically acceptable salt, stereoisomer, solvate, prodrug, isotopic derivative, or tautomer thereof.

In some embodiments, the compound is of Formula (II-A-I-G″):

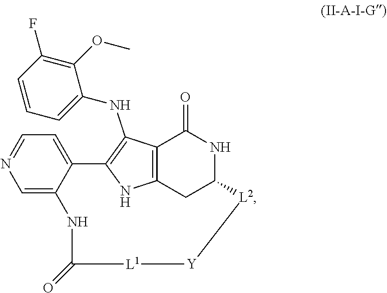

or a pharmaceutically acceptable salt, stereoisomer, solvate, prodrug, isotopic derivative, or tautomer thereof.

In some embodiments, the compound is of Formula (II-A-I-A-a):

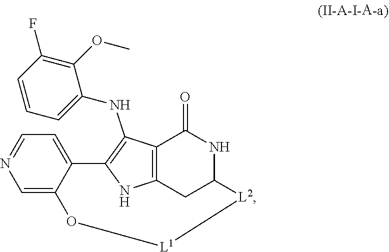

or a pharmaceutically acceptable salt, stereoisomer, solvate, prodrug, isotopic derivative, or tautomer thereof.

In some embodiments, the compound is of Formula (II-A-I-A-a′):

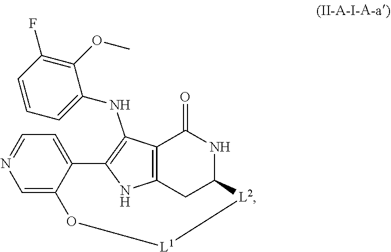

or a pharmaceutically acceptable salt, stereoisomer, solvate, prodrug, isotopic derivative, or tautomer thereof.

In some embodiments, the compound is of Formula (II-A-I-A-a″):

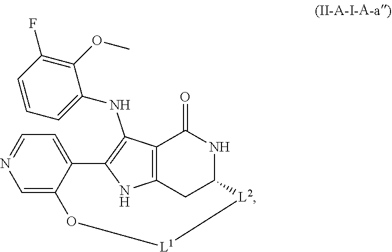

or a pharmaceutically acceptable salt, stereoisomer, solvate, prodrug, isotopic derivative, or tautomer thereof.

In some embodiments, the compound is of Formula (II-A-I-A-b):

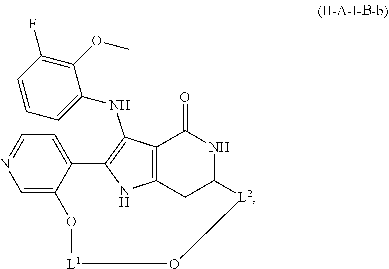

or a pharmaceutically acceptable salt, stereoisomer, solvate, prodrug, isotopic derivative, or tautomer thereof.

In some embodiments, the compound is of Formula (II-A-I-A-b′):

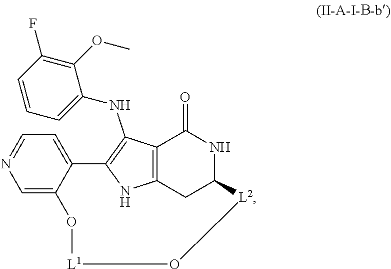

or a pharmaceutically acceptable salt, stereoisomer, solvate, prodrug, isotopic derivative, or tautomer thereof.

In some embodiments, the compound is of Formula (II-A-I-A-b″):

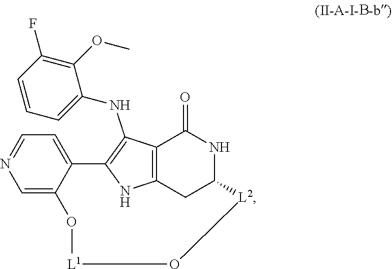

or a pharmaceutically acceptable salt, stereoisomer, solvate, prodrug, isotopic derivative, or tautomer thereof.

In some embodiments, the compound is of Formula (II-A-I-A-c):

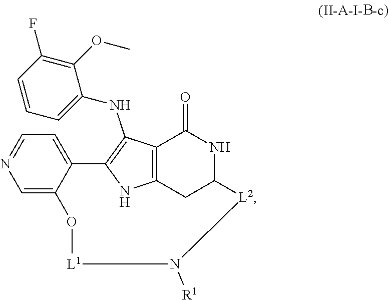

or a pharmaceutically acceptable salt, stereoisomer, solvate, prodrug, isotopic derivative, or tautomer thereof.

In some embodiments, the compound is of Formula (II-A-I-A-c′):

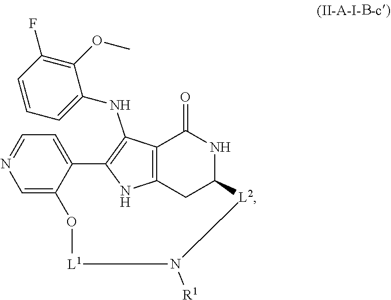

or a pharmaceutically acceptable salt, stereoisomer, solvate, pro rug, isotopic derivative, or tautomer thereof.

In some embodiments, the compound is of Formula (II-A-I-A-c″):

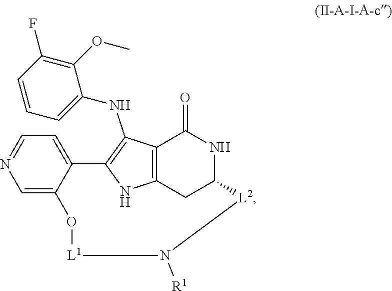

or a pharmaceutically acceptable salt, stereoisomer, solvate, prodrug, isotopic derivative, or tautomer thereof.

In some embodiments, the compound is of Formula (II-A-I-A-c-H):

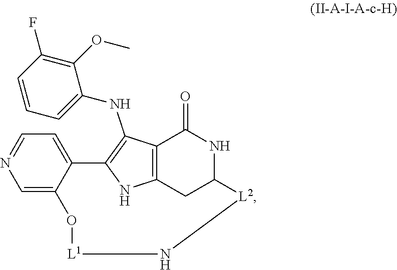

or a pharmaceutically acceptable salt, stereoisomer, solvate, prodrug, isotopic derivative, or tautomer thereof.

In some embodiments, the compound is of Formula (II-A-I-A-c-H′):

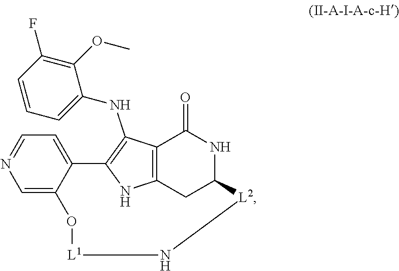

or a pharmaceutically acceptable salt, stereoisomer, solvate, prodrug, isotopic derivative, or tautomer thereof.

In some embodiments, the compound is of Formula (II-A-I-A-c-H″):

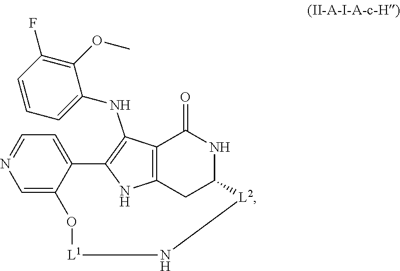

or a pharmaceutically acceptable salt, stereoisomer, solvate, prodrug, isotopic derivative, or tautomer thereof.

In some embodiments, the compound is of Formula (II-A-I-A-c-M):

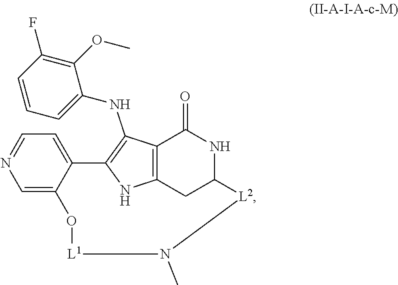

or a pharmaceutically acceptable salt, stereoisomer, solvate, prodrug, isotopic derivative, or tautomer thereof.

In some embodiments, the compound is of Formula (II-A-I-A-c-M′):

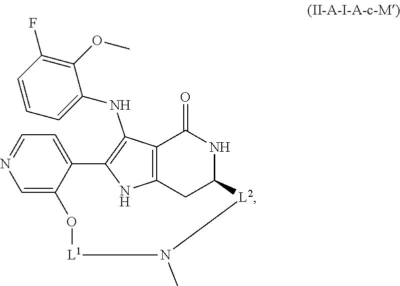

or a pharmaceutically acceptable salt, stereoisomer, solvate, prodrug, isotopic derivative, or tautomer thereof.

In some embodiments, the compound is of Formula (II-A-I-A-c-M″):

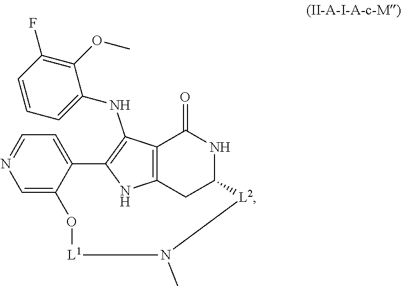

or a pharmaceutically acceptable salt, stereoisomer, solvate, prodrug, isotopic derivative, or tautomer thereof.

In some embodiments, the compound is of Formula (II-A-I-A-d):

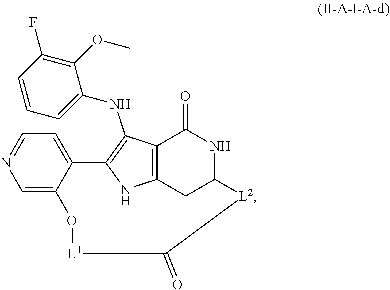

or a pharmaceutically acceptable salt, stereoisomer, solvate, prodrug, isotopic derivative, or tautomer thereof.

In some embodiments, the compound is of Formula (II-A-I-A-d′):

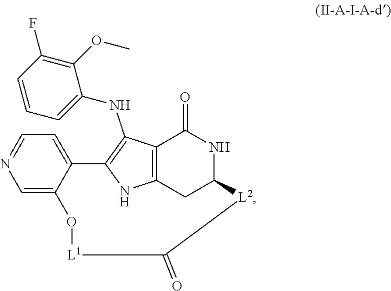

or a pharmaceutically acceptable salt, stereoisomer, solvate, prodrug, isotopic derivative, or tautomer thereof.

In some embodiments, the compound is of Formula (II-A-I-A-d″):

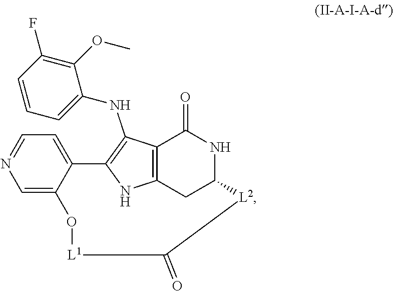

or a pharmaceutically acceptable salt, stereoisomer, solvate, prodrug, isotopic derivative, or tautomer thereof.

In some embodiments, the compound is of Formula (II-A-I-A-e):

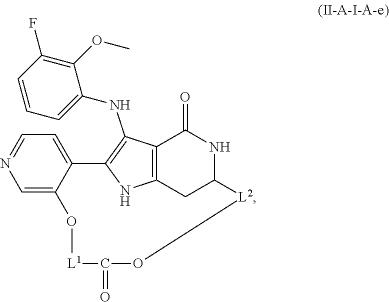

or a pharmaceutically acceptable salt, stereoisomer, solvate, prodrug, isotopic derivative, or tautomer thereof.

In some embodiments, the compound is of Formula (II-A-I-A-e′):

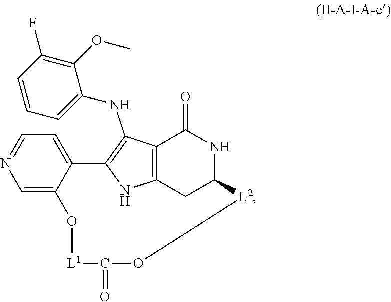

or a pharmaceutically acceptable salt, stereoisomer, solvate, prodrug, isotopic derivative, or tautomer thereof.

In some embodiments, the compound is of Formula (II-A-I-A-e″):

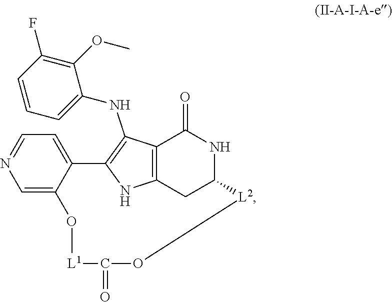

or a pharmaceutically acceptable salt, stereoisomer, solvate, prodrug, isotopic derivative, or tautomer thereof.

In some embodiments, the compound is of Formula (II-A-I-A-f):

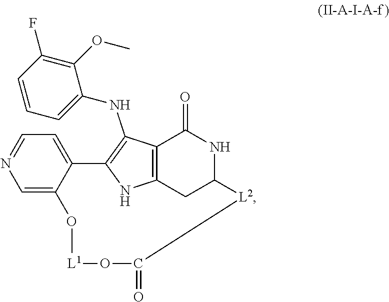

or a pharmaceutically acceptable salt, stereoisomer, solvate, prodrug, isotopic derivative, or tautomer thereof.

In some embodiments, the compound is of Formula (II-A-I-A-f′):

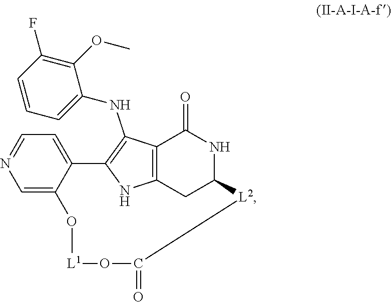

or a pharmaceutically acceptable salt, stereoisomer, solvate, prodrug, isotopic derivative, or tautomer thereof.

In some embodiments, the compound is of Formula (II-A-I-A-f′):

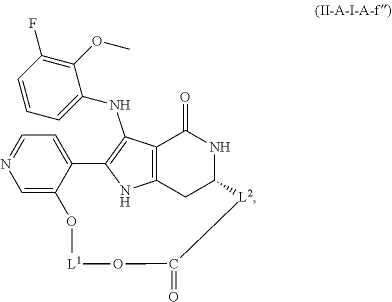

or a pharmaceutically acceptable salt, stereoisomer, solvate, prodrug, isotopic derivative, or tautomer thereof.

In some embodiments, the compound is of Formula (II-A-I-A-g):

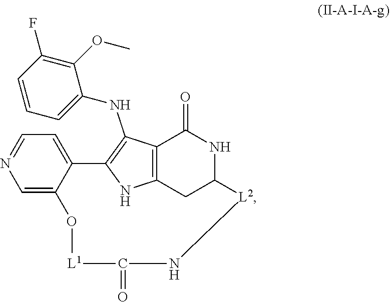

or a pharmaceutically acceptable salt, stereoisomer, solvate, prodrug, isotopic derivative, or tautomer thereof.

In some embodiments, the compound is of Formula (II-A-I-A-g′):

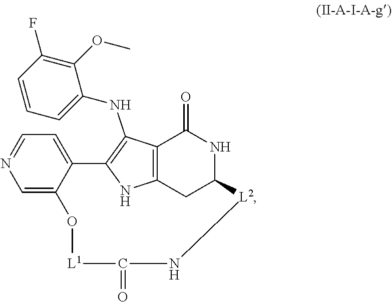

or a pharmaceutically acceptable salt, stereoisomer, solvate, prodrug, isotopic derivative, or tautomer thereof.

In some embodiments, the compound is of Formula (II-A-I-A-g″):

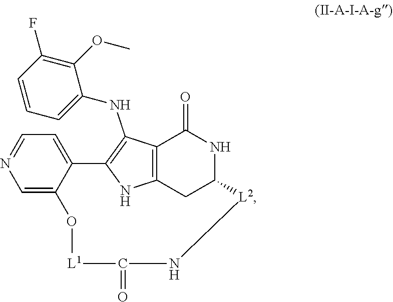

or a pharmaceutically acceptable salt, stereoisomer, solvate, prodrug, isotopic derivative, or tautomer thereof.

In some embodiments, the compound is of Formula (II-A-I-A-h):

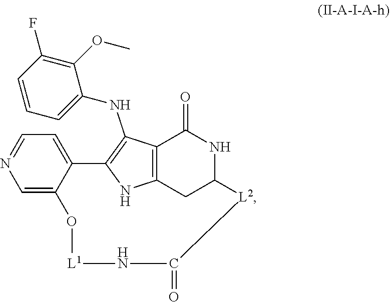

or a pharmaceutically acceptable salt, stereoisomer, solvate, prodrug, isotopic derivative, or tautomer thereof.

In some embodiments, the compound is of Formula (II-A-I-A-h′):

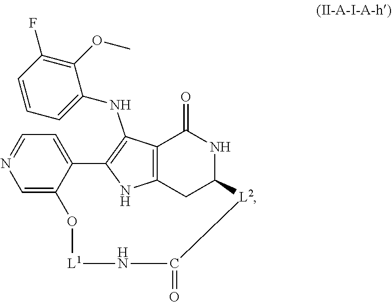

or a pharmaceutically acceptable salt, stereoisomer, solvate, prodrug, isotopic derivative, or tautomer thereof.

In some embodiments, the compound is of Formula (II-A-I-A-h″):

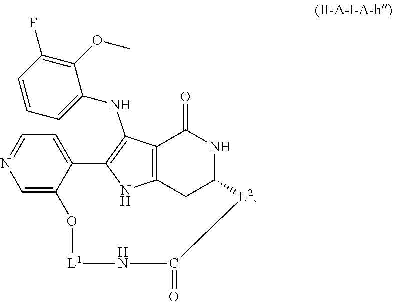

or a pharmaceutically acceptable salt, stereoisomer, solvate, prodrug, isotopic derivative, or tautomer thereof.

In some embodiments, the compound is of Formula (II-A-I-A-i):

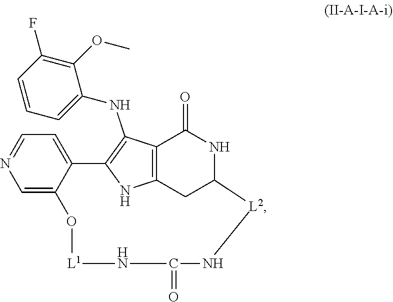

or a pharmaceutically acceptable salt, stereoisomer, solvate, prodrug, isotopic derivative, or tautomer thereof.

In some embodiments, the compound is of Formula (II-A-I-A-i′):

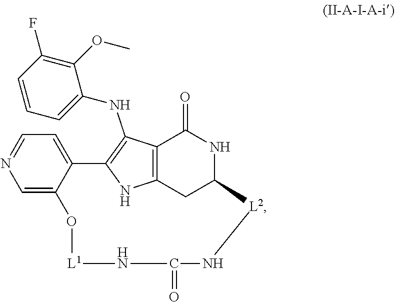

or a pharmaceutically acceptable salt, stereoisomer, solvate, prodrug, isotopic derivative, or tautomer thereof.

In some embodiments, the compound is of Formula (II-A-I-A-i″):

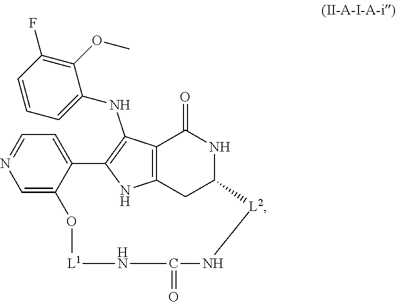

or a pharmaceutically acceptable salt, stereoisomer, solvate, prodrug, isotopic derivative, or tautomer thereof.

In some embodiments, the compound is of Formula (II-A-I-A-j):

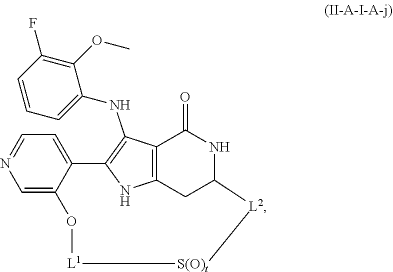

or a pharmaceutically acceptable salt, stereoisomer, solvate, prodrug, isotopic derivative, or tautomer thereof wherein t is 0, 1, or 2 and all other variables are as defined herein.

In some embodiments, the compound is of Formula (II-A-I-A-i′):

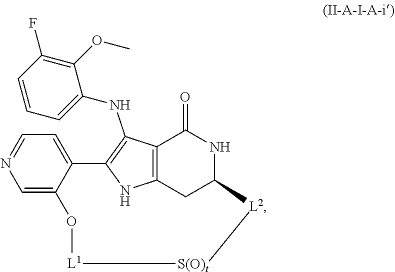

or a pharmaceutically acceptable salt, stereoisomer, solvate, prodrug, isotopic derivative, or tautomer thereof wherein t is 0, 1, or 2 and all other variables are as defined herein.

In some embodiments, the compound is of Formula (II-A-I-A-i″):

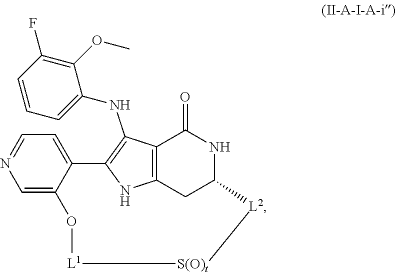

or a pharmaceutically acceptable salt, stereoisomer, solvate, prodrug, isotopic derivative, or tautomer thereof wherein t is 0, 1, or 2 and all other variables are as defined herein.

In some embodiments, the compound is of Formula (II-A-I-A-k):

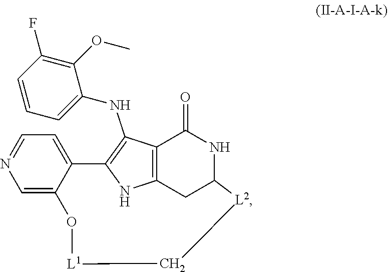

or a pharmaceutically acceptable salt, stereoisomer, solvate, prodrug, isotopic derivative, or tautomer thereof.

In some embodiments, the compound is of Formula (II-A-I-A-k′):

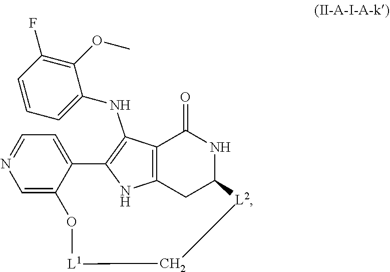

or a pharmaceutically acceptable salt, stereoisomer, solvate, prodrug, isotopic derivative, or tautomer thereof.

In some embodiments, the compound is of Formula (II-A-I-A-k″):

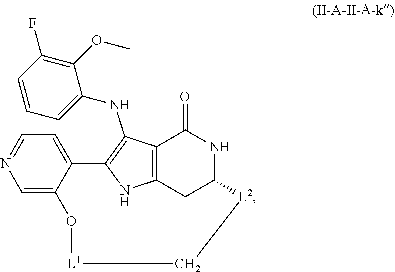

or a pharmaceutically acceptable salt, stereoisomer, solvate, prodrug, isotopic derivative, or tautomer thereof.

In some embodiments, the compound is of Formula (II-A-I-B-a):

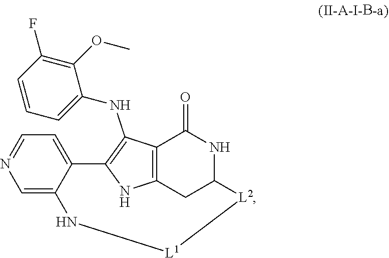

or a pharmaceutically acceptable salt, stereoisomer, solvate, prodrug, isotopic derivative, or tautomer thereof.

In some embodiments, the compound is of Formula (II-A-I-B-a′):

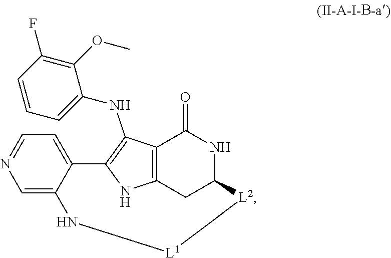

or a pharmaceutically acceptable salt, stereoisomer, solvate, prodrug, isotopic derivative, or tautomer thereof.

In some embodiments, the compound is of Formula (II-A-I-B-a″):

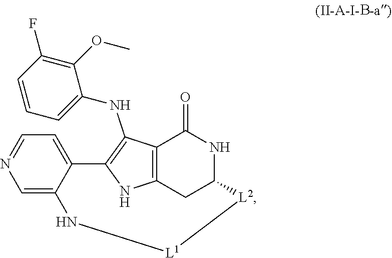

or a pharmaceutically acceptable salt, stereoisomer, solvate, prodrug, isotopic derivative, or tautomer thereof.

In some embodiments, the compound is of Formula (II-A-I-B-b):

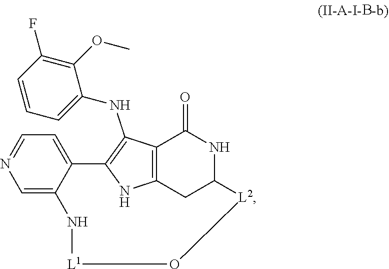

or a pharmaceutically acceptable salt, stereoisomer, solvate, prodrug, isotopic derivative, or tautomer thereof.

In some embodiments, the compound is of Formula (II-A-I-B-b′):

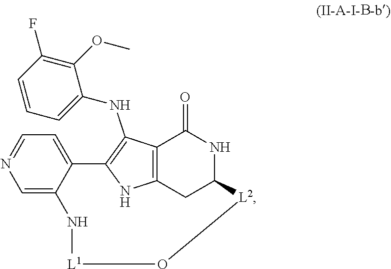

or a pharmaceutically acceptable salt, stereoisomer, solvate, prodrug, isotopic derivative, or tautomer thereof.

In some embodiments, the compound is of Formula (II-A-I-B-b″):

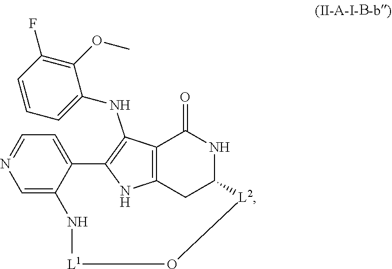

or a pharmaceutically acceptable salt, stereoisomer, solvate, prodrug, isotopic derivative, or tautomer thereof.

In some embodiments, the compound is of Formula (II-A-I-B-c):

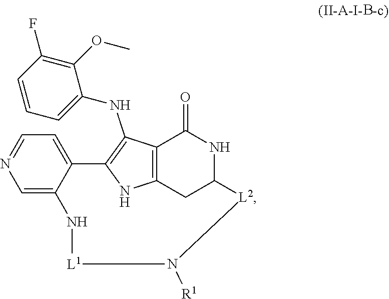

or a pharmaceutically acceptable salt, stereoisomer, solvate, prodrug, isotopic derivative, or tautomer thereof.

In some embodiments, the compound is of Formula (II-A-I-B-c′):

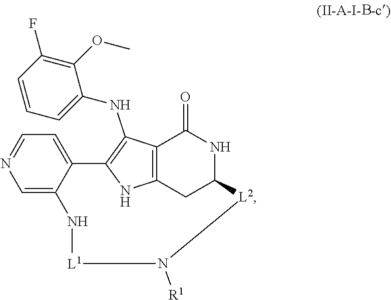

or a pharmaceutically acceptable salt, stereoisomer, solvate, prodrug, isotopic derivative, or tautomer thereof.

In some embodiments, the compound is of Formula (II-A-I-B-c″):

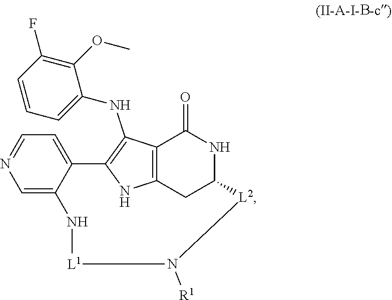

or a pharmaceutically acceptable salt, stereoisomer, solvate, prodrug, isotopic derivative, or tautomer thereof.

In some embodiments, the compound is of Formula (II-A-I-B-c-H):

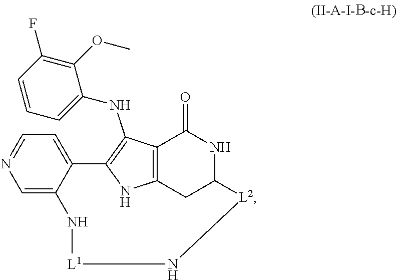

or a pharmaceutically acceptable salt, stereoisomer, solvate, prodrug, isotopic derivative, or tautomer thereof.

In some embodiments, the compound is of Formula (II-A-I-B-c-H′):

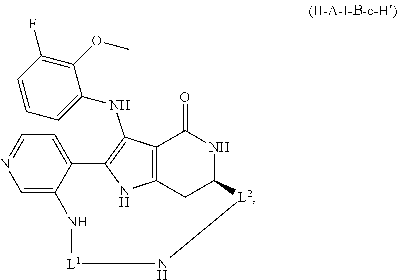

or a pharmaceutically acceptable salt, stereoisomer, solvate, prodrug, isotopic derivative, or tautomer thereof.

In some embodiments, the compound is of Formula (II-A-I-B-c-H″):

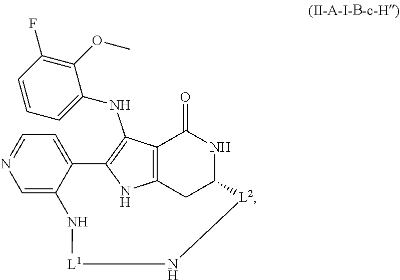

or a pharmaceutically acceptable salt, stereoisomer, solvate, prodrug, isotopic derivative, or tautomer thereof.

In some embodiments, the compound is of Formula (II-A-I-B-c-M):

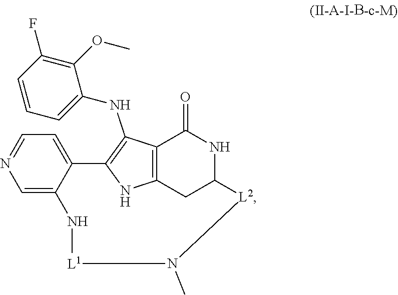

or a pharmaceutically acceptable salt, stereoisomer, solvate, prodrug, isotopic derivative, or tautomer thereof.

In some embodiments, the compound is of Formula (II-A-I-B-c-M′):

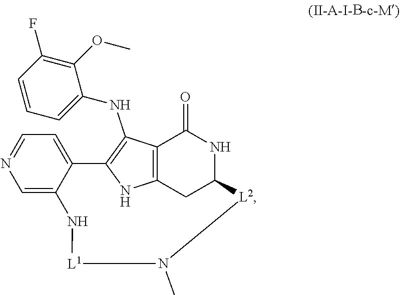

or a pharmaceutically acceptable salt, stereoisomer, solvate, prodrug, isotopic derivative, or tautomer thereof.

In some embodiments, the compound is of Formula (II-A-I-B-c-M″):

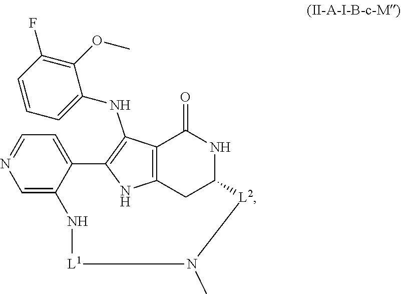

or a pharmaceutically acceptable salt, stereoisomer, solvate, prodrug, isotopic derivative, or tautomer thereof.

In some embodiments, the compound is of Formula (II-A-I-B-d):

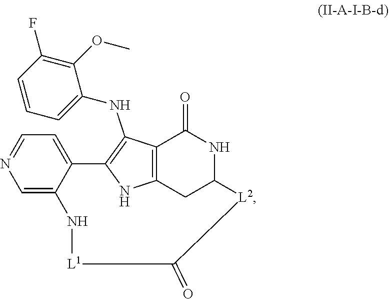

or a pharmaceutically acceptable salt, stereoisomer, solvate, prodrug, isotopic derivative, or tautomer thereof.

In some embodiments, the compound is of Formula (II-A-I-B-d′):

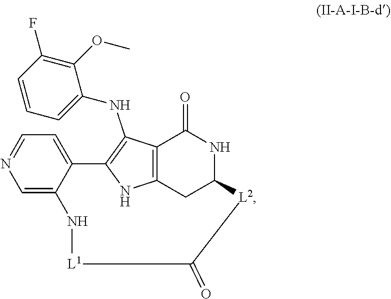

or a pharmaceutically acceptable salt, stereoisomer, solvate, prodrug, isotopic derivative, or tautomer thereof.

In some embodiments, the compound is of Formula (II-A-I-B-d″):

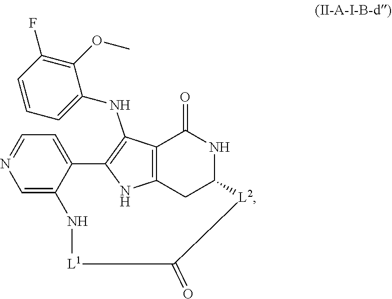

or a pharmaceutically acceptable salt, stereoisomer, solvate, prodrug, isotopic derivative, or tautomer thereof.

In some embodiments, the compound is of Formula (II-A-I-B-e):

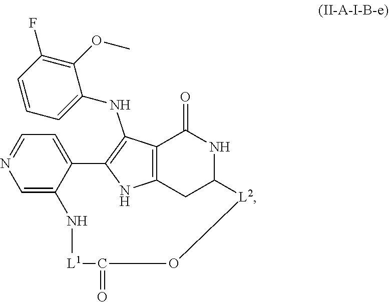

or a pharmaceutically acceptable salt, stereoisomer, solvate, prodrug, isotopic derivative, or tautomer thereof.

In some embodiments, the compound is of Formula (II-A-I-B-e′):

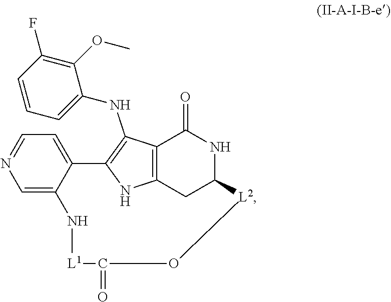

or a pharmaceutically acceptable salt, stereoisomer, solvate, prodrug, isotopic derivative, or tautomer thereof.

In some embodiments, the compound is of Formula (II-A-I-B-e″):

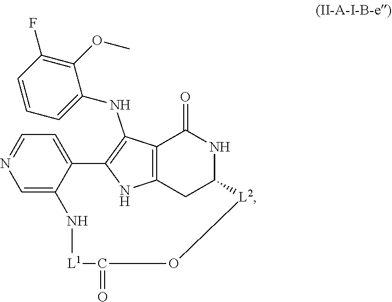

or a pharmaceutically acceptable salt, stereoisomer, solvate, prodrug, isotopic derivative, or tautomer thereof.

In some embodiments, the compound is of Formula (II-A-I-B-f):

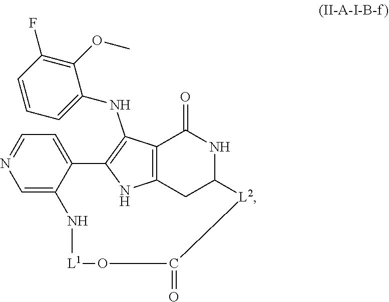

or a pharmaceutically acceptable salt, stereoisomer, solvate, prodrug, isotopic derivative, or tautomer thereof.

In some embodiments, the compound is of Formula (II-A-I-B-f′):

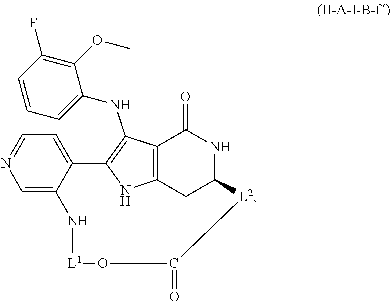

or a pharmaceutically acceptable salt, stereoisomer, solvate, prodrug, isotopic derivative, or tautomer thereof.

In some embodiments, the compound is of Formula (II-A-I-B-f′):

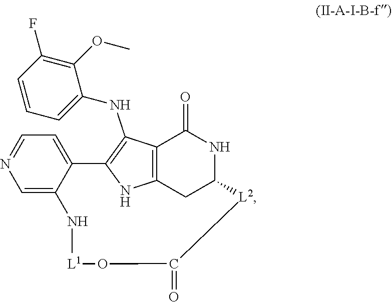

or a pharmaceutically acceptable salt, stereoisomer, solvate, prodrug, isotopic derivative, or tautomer thereof.

In some embodiments, the compound is of Formula (II-A-I-B-g):

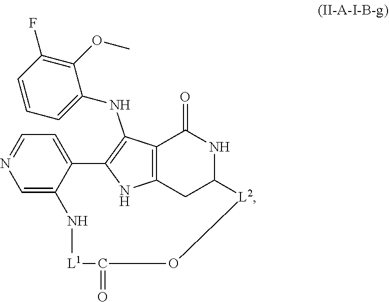

or a pharmaceutically acceptable salt, stereoisomer, solvate, prodrug, isotopic derivative, or tautomer thereof.

In some embodiments, the compound is of Formula (II-A-I-B-g′):

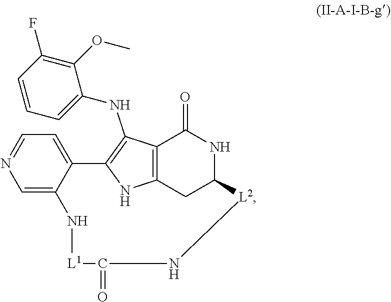

or a pharmaceutically acceptable salt, stereoisomer, solvate, prodrug, isotopic derivative, or tautomer thereof.

In some embodiments, the compound is of Formula (II-A-I-B-g″):

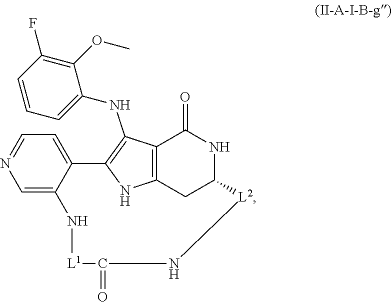

or a pharmaceutically acceptable salt, stereoisomer, solvate, prodrug, isotopic derivative, or tautomer thereof.

In some embodiments, the compound is of Formula (II-A-I-B-h):

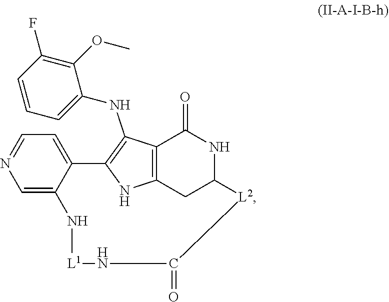

or a pharmaceutically acceptable salt, stereoisomer, solvate, prodrug, isotopic derivative, or tautomer thereof.

In some embodiments, the compound is of Formula (II-A-I-B-h′):

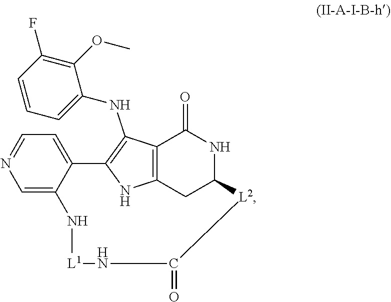

or a pharmaceutically acceptable salt, stereoisomer, solvate, prodrug, isotopic derivative, or tautomer thereof.

In some embodiments, the compound is of Formula (II-A-I-B-h″):

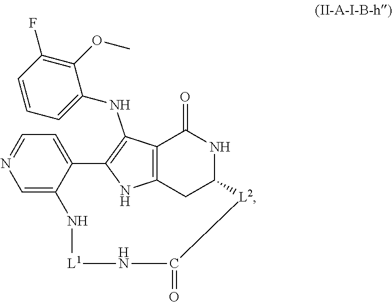

or a pharmaceutically acceptable salt, stereoisomer, solvate, prodrug, isotopic derivative, or tautomer thereof.

In some embodiments, the compound is of Formula (II-A-I-B-i):

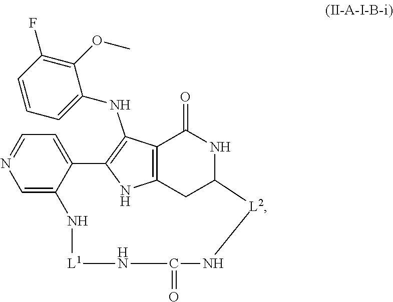

or a pharmaceutically acceptable salt, stereoisomer, solvate, prodrug, isotopic derivative, or tautomer thereof.

In some embodiments, the compound is of Formula (II-A-I-B-i′):

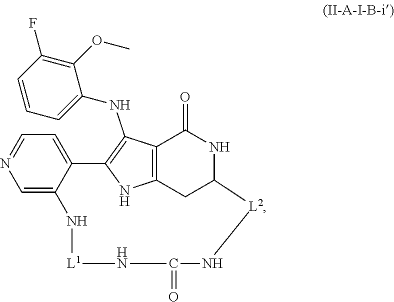

or a pharmaceutically acceptable salt, stereoisomer, solvate, prodrug, isotopic derivative, or tautomer thereof.

In some embodiments, the compound is of Formula (II-A-I-B-i″):

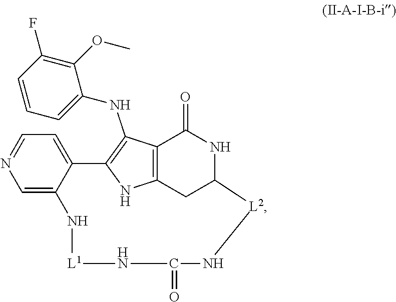

or a pharmaceutically acceptable salt, stereoisomer, solvate, prodrug, isotopic derivative, or tautomer thereof.

In some embodiments, the compound is of Formula (II-A-I-B-j):

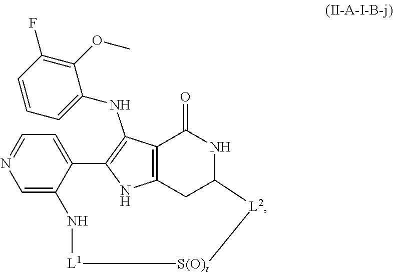

or a pharmaceutically acceptable salt, stereoisomer, solvate, prodrug, isotopic derivative, or tautomer thereof wherein t is 0, 1, or 2 and all other variables are as defined herein.

In some embodiments, the compound is of Formula (II-A-I-B-i′):

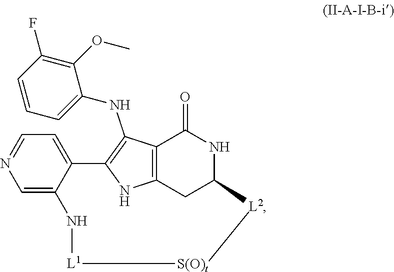

or a pharmaceutically acceptable salt, stereoisomer, solvate, prodrug, isotopic derivative, or tautomer thereof wherein t is 0, 1, or 2 and all other variables are as defined herein.

In some embodiments, the compound is of Formula (II-A-I-B-i″):

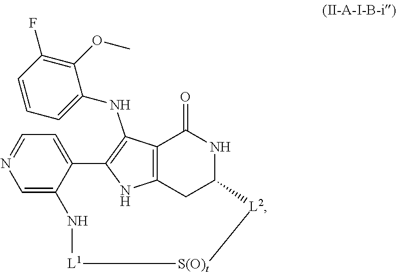

or a pharmaceutically acceptable salt, stereoisomer, solvate, prodrug, isotopic derivative, or tautomer thereof wherein t is 0, 1, or 2 and all other variables are as defined herein.

In some embodiments, the compound is of Formula (II-A-I-B-k):

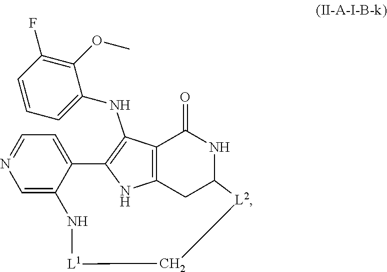

or a pharmaceutically acceptable salt, stereoisomer, solvate, prodrug, isotopic derivative, or tautomer thereof.

In some embodiments, the compound is of Formula (II-A-I-B-k′):

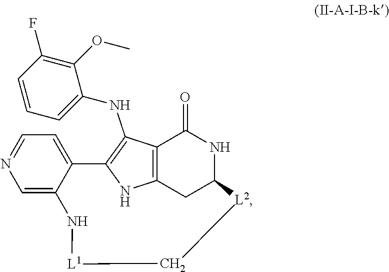

or a pharmaceutically acceptable salt, stereoisomer, solvate, prodrug, isotopic derivative, or tautomer thereof.

In some embodiments, the compound is of Formula (II-A-I-B-k″):

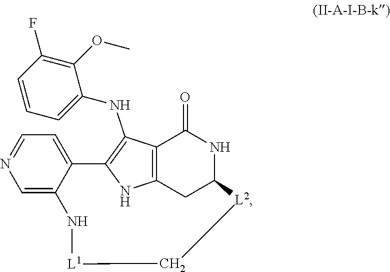

or a pharmaceutically acceptable salt, stereoisomer, solvate, prodrug, isotopic derivative, or tautomer thereof.

In some embodiments, the compound is of Formula (II-A-I-A-a-1):

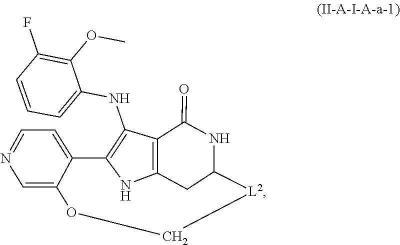

or a pharmaceutically acceptable salt, stereoisomer, solvate, prodrug, isotopic derivative, or tautomer thereof.

In some embodiments, the compound is of Formula (II-A-I-A-a-1′) or (II-A-I-A-a-1″):

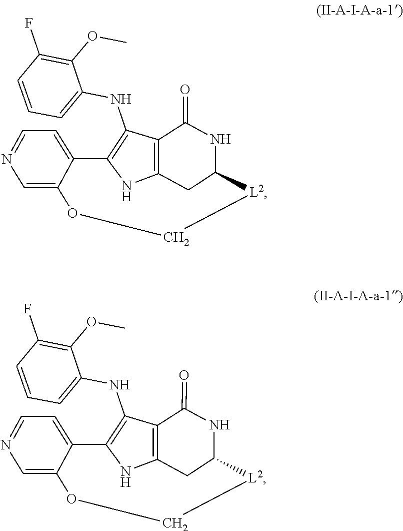

or a pharmaceutically acceptable salt, stereoisomer, solvate, prodrug, isotopic derivative, or tautomer thereof.

In some embodiments, the compound is of Formula (II-A-I-A-a-2):

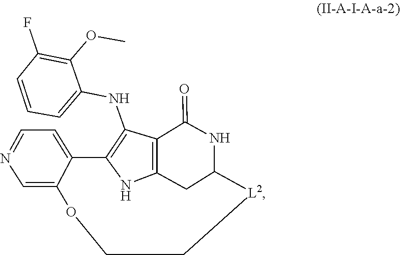

or a pharmaceutically acceptable salt, stereoisomer, solvate, prodrug, isotopic derivative, or tautomer thereof.

In some embodiments, the compound is of Formula (II-A-I-A-a-2′) or (II-A-I-A-a-2″):

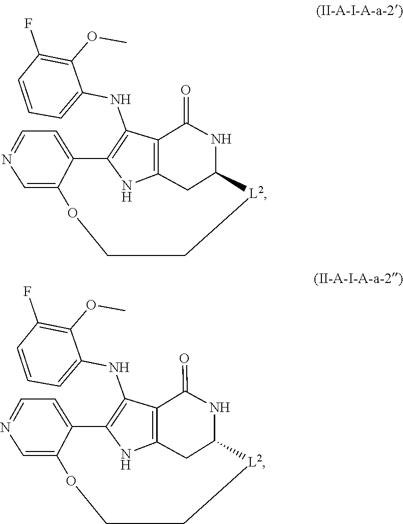

or a pharmaceutically acceptable salt, stereoisomer, solvate, prodrug, isotopic derivative, or tautomer thereof.

In some embodiments, the compound is of Formula (II-A-I-A-a-3):

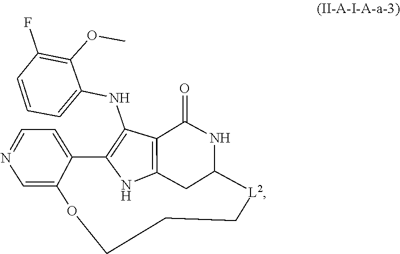

or a pharmaceutically acceptable salt, stereoisomer, solvate, prodrug, isotopic derivative, or tautomer thereof.

In some embodiments, the compound is of Formula (I-A-I-A-a-3′) or (I-A-I-A-a-3″):

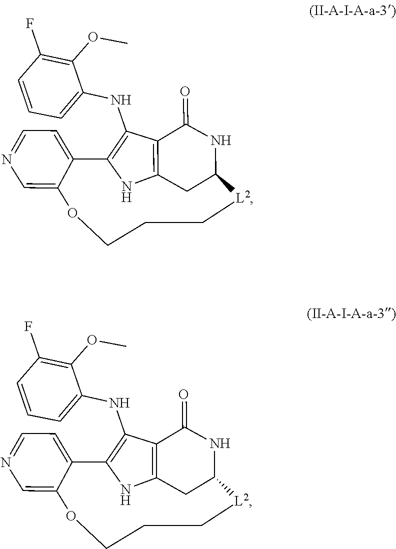

or a pharmaceutically acceptable salt, stereoisomer, solvate, prodrug, isotopic derivative, or tautomer thereof.

In some embodiments, the compound is of Formula (II-A-I-A-a-4):

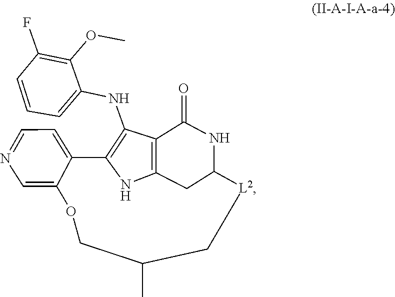

or a pharmaceutically acceptable salt, stereoisomer, solvate, prodrug, isotopic derivative, or tautomer thereof.

In some embodiments, the compound is of Formula (II-A-I-A-a-4′) or (II-A-I-A-a-4″):

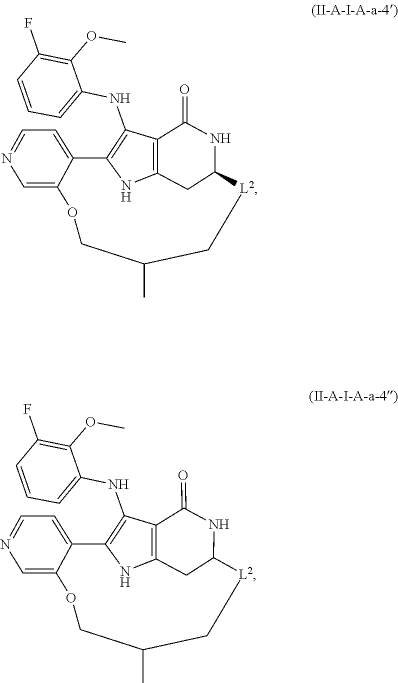

or a pharmaceutically acceptable salt, stereoisomer, solvate, prodrug, isotopic derivative, or tautomer thereof.

In some embodiments, the compound is of Formula (II-A-I-A-a-5):

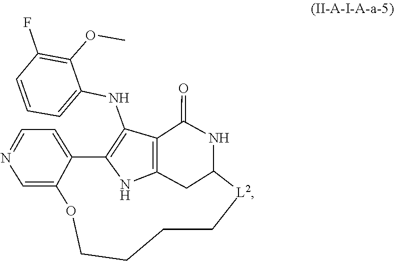

or a pharmaceutically acceptable salt, stereoisomer, solvate, prodrug, isotopic derivative, or tautomer thereof.

In some embodiments, the compound is of Formula (II-A-I-A-a-5′) or (II-A-I-A-a-5″):

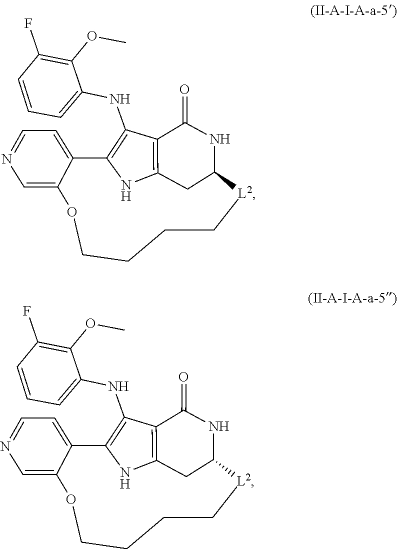

or a pharmaceutically acceptable salt, stereoisomer, solvate, prodrug, isotopic derivative, or tautomer thereof.

In some embodiments, the compound is of Formula (II-A-I-A-a-6):

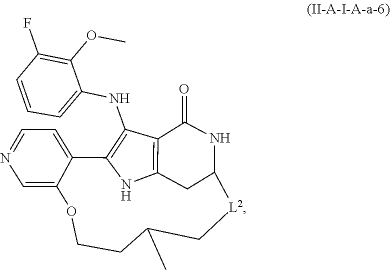

or a pharmaceutically acceptable salt, stereoisomer, solvate, prodrug, isotopic derivative, or tautomer thereof.

In some embodiments, the compound is of Formula (TT-I-A-a-6′) or (II-A-T-A-a-6″)

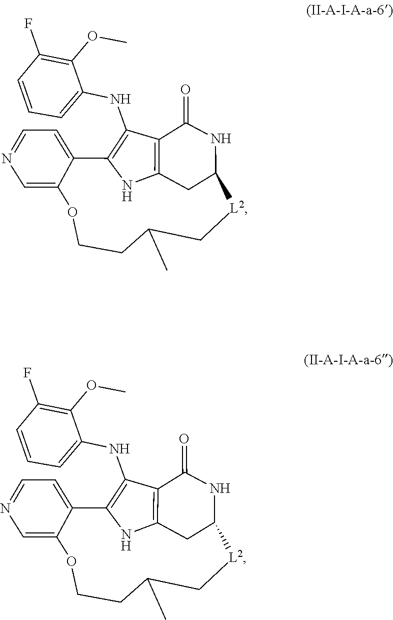

or a pharmaceutically acceptable salt, stereoisomer, solvate, prodrug, isotopic derivative, or tautomer thereof.

In some embodiments, the compound is of Formula (II-A-I-A-a-7):

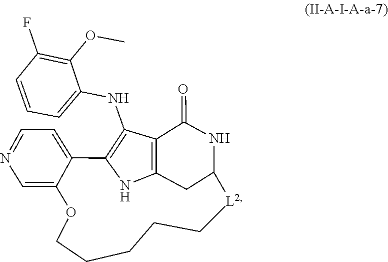

or a pharmaceutically acceptable salt, stereoisomer, solvate, prodrug, isotopic derivative, or tautomer thereof.

In some embodiments, the compound is of Formula (II-A-I-A-a-7′) or (II-A-I-A-a-7″):

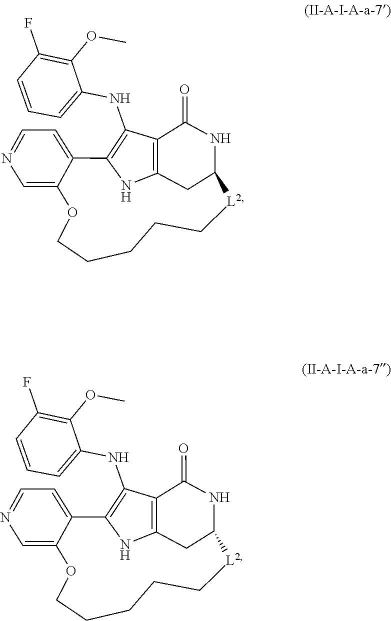

or a pharmaceutically acceptable salt, stereoisomer, solvate, prodrug, isotopic derivative, or tautomer thereof.

In some embodiments, the compound is of Formula (II-A-I-A-a-8):

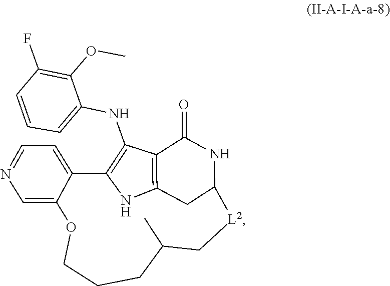

or a pharmaceutically acceptable salt, stereoisomer, solvate, prodrug, isotopic derivative, or tautomer thereof.

In some embodiments, the compound is of Formula (II-A-I-A-a-8′) or (II-A-I-A-a-8″):

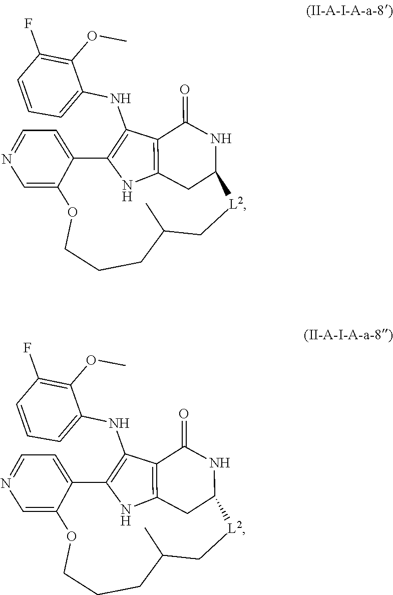

or a pharmaceutically acceptable salt, stereoisomer, solvate, prodrug, isotopic derivative, or tautomer thereof.

In some embodiments, the compound is of Formula (II-A-I-A-a-9):

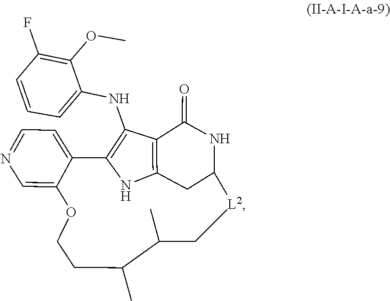

or a pharmaceutically acceptable salt, stereoisomer, solvate, prodrug, isotopic derivative, or tautomer thereof.

In some embodiments, the compound is of Formula (II-A-I-A-a-9′) or (II-A-I-A-a-9″):

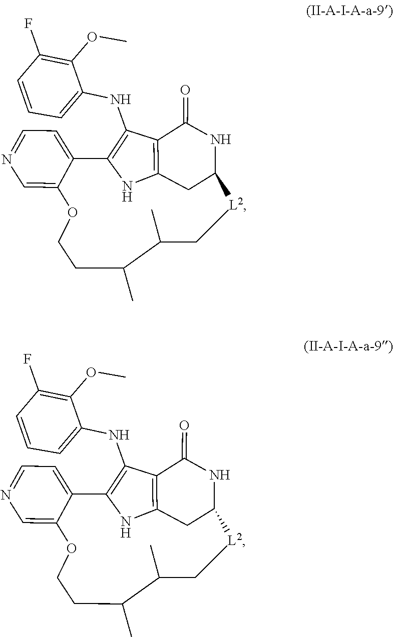

or a pharmaceutically acceptable salt, stereoisomer, solvate, prodrug, isotopic derivative, or tautomer thereof.

In some embodiments, the compound is of Formula (I-A-I-A-a-10):

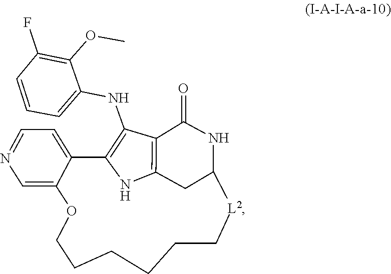

or a pharmaceutically acceptable salt, stereoisomer, solvate, prodrug, isotopic derivative, or tautomer thereof.

In some embodiments, the compound is of Formula (I-A-I-A-a-10′) or (I-A-I-A-a-10″):

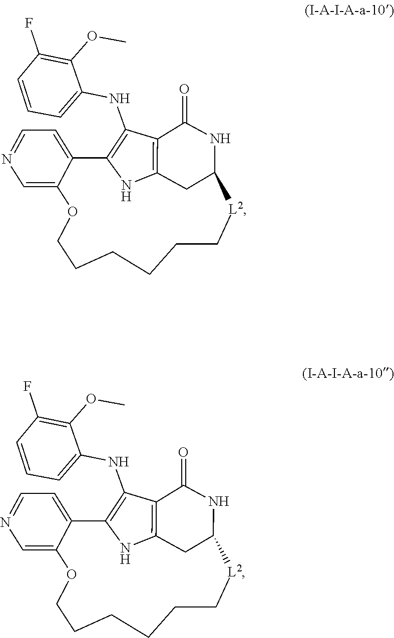

or a pharmaceutically acceptable salt, stereoisomer, solvate, prodrug, isotopic derivative, or tautomer thereof.

In some embodiments, the compound is of Formula (II-A-I-A-a-11):

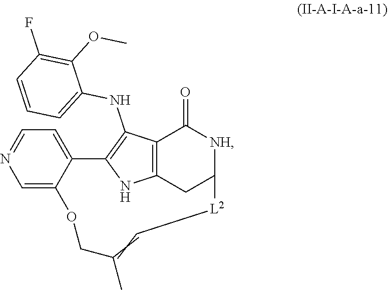

or a pharmaceutically acceptable salt, stereoisomer, solvate, prodrug, isotopic derivative, or tautomer thereof.

In some embodiments, the compound is of Formula (E-II-A-I-A-a-11) or (Z-II-A-I-A-a-11):

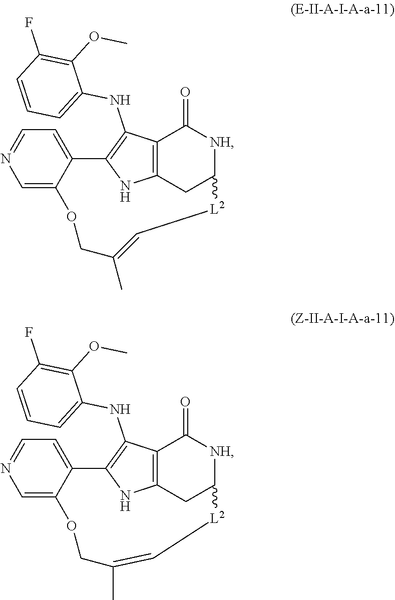

or a pharmaceutically acceptable salt, stereoisomer, solvate, prodrug, isotopic derivative, or tautomer thereof.

In some embodiments, the compound is of Formula (II-A-I-A-a-12):

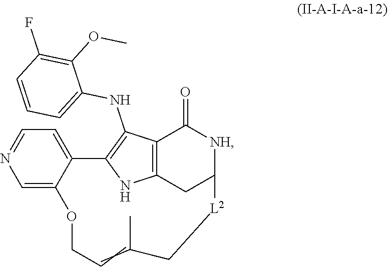

or a pharmaceutically acceptable salt, stereoisomer, solvate, prodrug, isotopic derivative, or tautomer thereof.

In some embodiments, the compound is of Formula (E-II-A-I-A-a-12) or (Z-II-A-I-A-a-12):

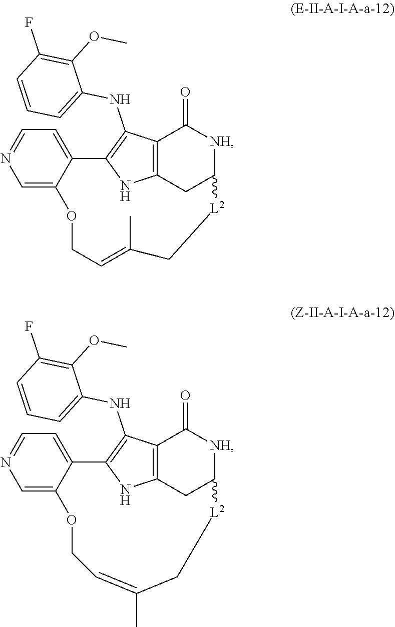

or a pharmaceutically acceptable salt, stereoisomer, solvate, prodrug, isotopic derivative, or tautomer thereof.

In some embodiments, the compound is of Formula (II-A-I-A-a-13):

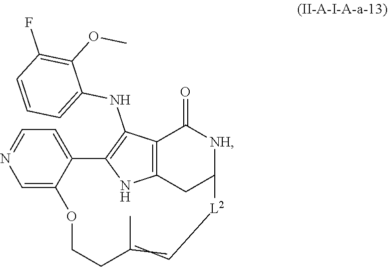

or a pharmaceutically acceptable salt, stereoisomer, solvate, prodrug, isotopic derivative, or tautomer thereof.

In some embodiments, the compound is of Formula (E-II-A-I-A-a-13) or (Z-II-A-I-A-a-F

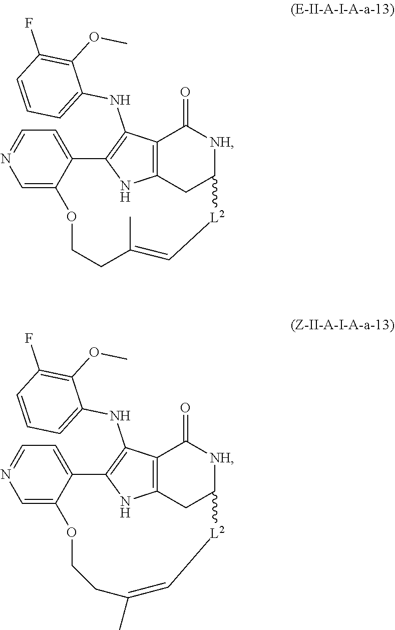

or a pharmaceutically acceptable salt, stereoisomer, solvate, prodrug, isotopic derivative, or tautomer thereof.

In some embodiments, the compound is of Formula (II-A-I-A-a-14):

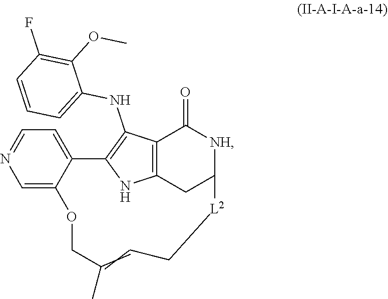

or a pharmaceutically acceptable salt, stereoisomer, solvate, prodrug, isotopic derivative, or tautomer thereof.

In some embodiments, the compound is of Formula (E-II-A-I-A-a-14) or (Z-II-A-I-A-a-14):

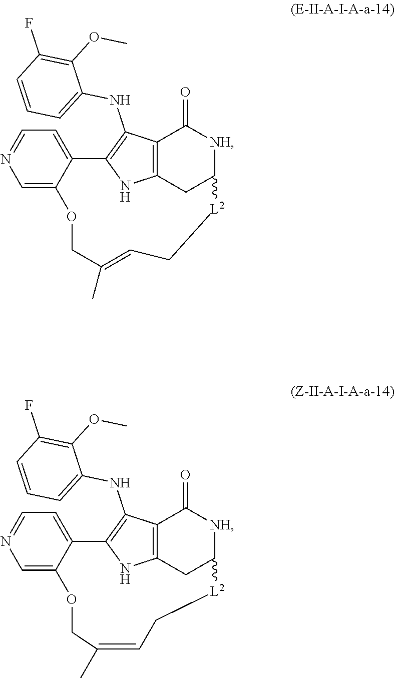

or a pharmaceutically acceptable salt, stereoisomer, solvate, prodrug, isotopic derivative, or tautomer thereof.

In some embodiments, the compound is of Formula (II-A-I-A-a-15):

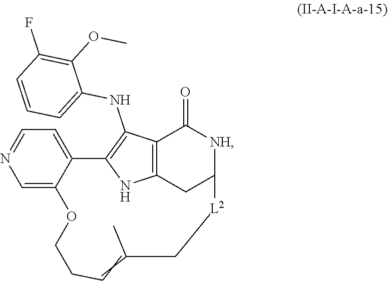

or a pharmaceutically acceptable salt, stereoisomer, solvate, prodrug, isotopic derivative, or tautomer thereof.

In some embodiments, the compound is of Formula (E-II-A-I-A-a-15) or (Z-II-A-I-A-a-15):

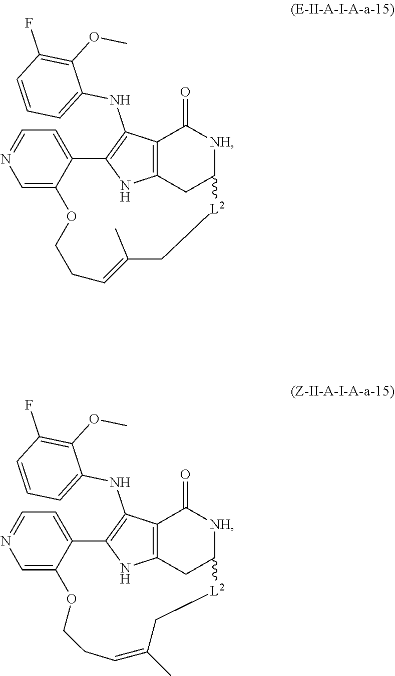

or a pharmaceutically acceptable salt, stereoisomer, solvate, prodrug, isotopic derivative, or tautomer thereof.

In some embodiments, the compound is of Formula (II-A-I-A-a-16):

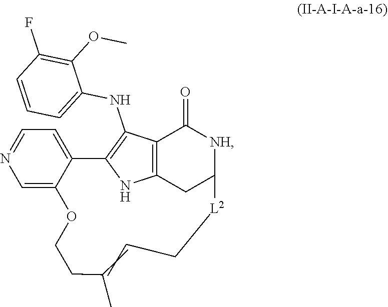

or a pharmaceutically acceptable salt, stereoisomer, solvate, prodrug, isotopic derivative, or tautomer thereof.

In some embodiments, the compound is of Formula (E-II-A-I-A-a-16) or (Z-II-A-I-A-a-16):

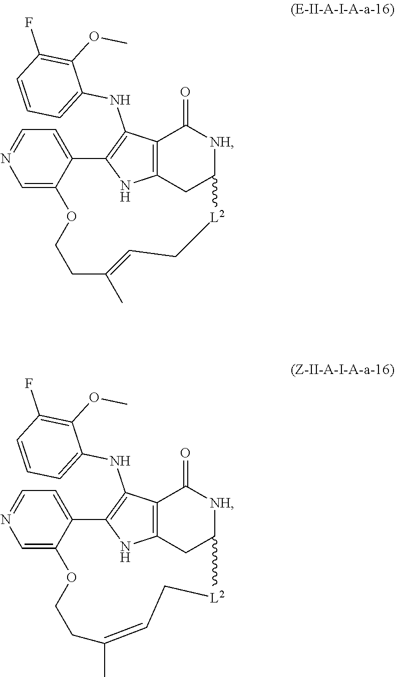

or a pharmaceutically acceptable salt, stereoisomer, solvate, prodrug, isotopic derivative, or tautomer thereof.

In some embodiments, the compound is of Formula (II-A-I-A-a-17):

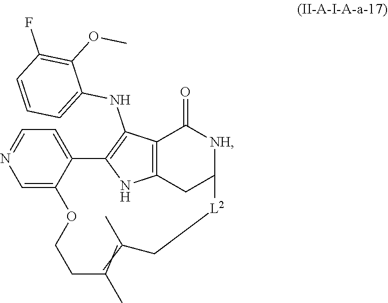

or a pharmaceutically acceptable salt, stereoisomer, solvate, prodrug, isotopic derivative, or tautomer thereof.

In some embodiments, the compound is of Formula (E-II-A-I-A-a-17) or (E-II-A-I-A-a-17):

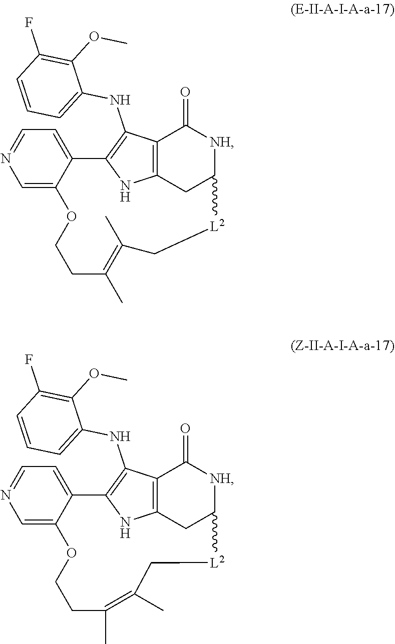

or a pharmaceutically acceptable salt, stereoisomer, solvate, prodrug, isotopic derivative, or tautomer thereof.

In some embodiments, the compound is of Formula (II-A-I-A-a-18):

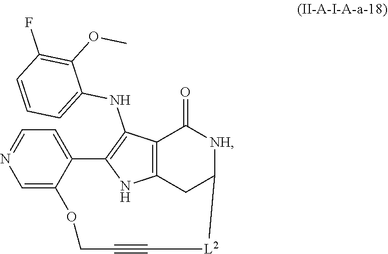

or a pharmaceutically acceptable salt, stereoisomer, solvate, prodrug, isotopic derivative, or tautomer thereof.

In some embodiments, the compound is of Formula (II-A-I-A-a-18′) or (II-A-I-A-a-18″):

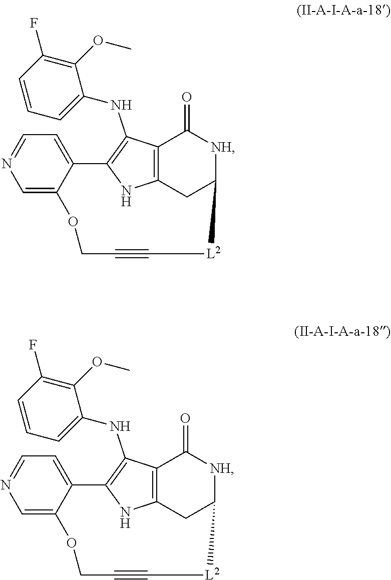

or a pharmaceutically acceptable salt, stereoisomer, solvate, prodrug, isotopic derivative, or tautomer thereof.

In some embodiments, the compound is of Formula (II-A-I-A-a-19):

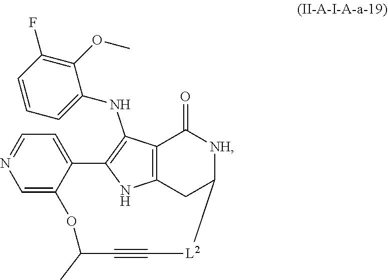

or a pharmaceutically acceptable salt, stereoisomer, solvate, prodrug, isotopic derivative, or tautomer thereof.

In some embodiments, the compound is of Formula (II-A-I-A-a-19′) or (II-A-I-A-a-19″):

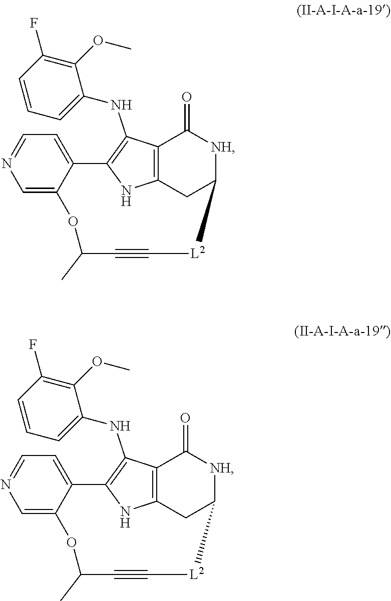

or a pharmaceutically acceptable salt, stereoisomer, solvate, prodrug, isotopic derivative, or tautomer thereof.

In some embodiments, the compound is of Formula (II-A-I-A-a-20):

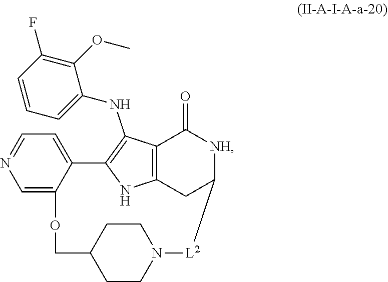

or a pharmaceutically acceptable salt, stereoisomer, solvate, prodrug, isotopic derivative, or tautomer thereof.

In some embodiments, the compound is of Formula (II-A-I-A-a-20′) or (II-A-I-A-a-20″):

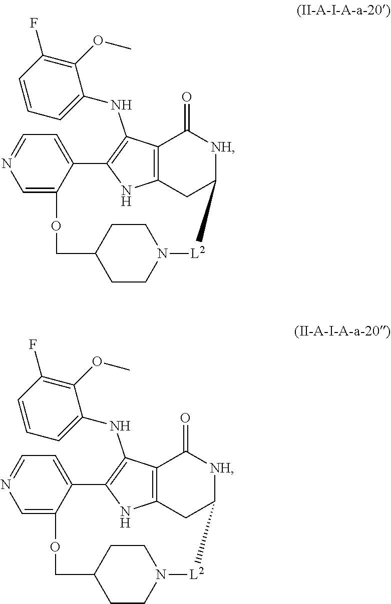

or a pharmaceutically acceptable salt, stereoisomer, solvate, prodrug, isotopic derivative, or tautomer thereof.

In some embodiments, the compound is of Formula (II-A-I-A-a-21):

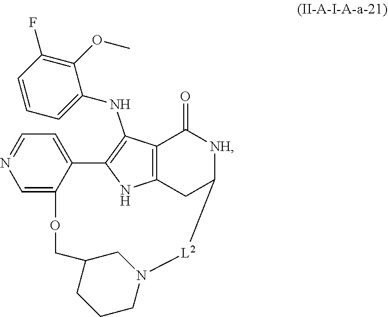

or a pharmaceutically acceptable salt, stereoisomer, solvate, prodrug, isotopic derivative, or tautomer thereof.

In some embodiments, the compound is of Formula (II-A-I-A-a-21′) or (II-A-I-A-a-21″):

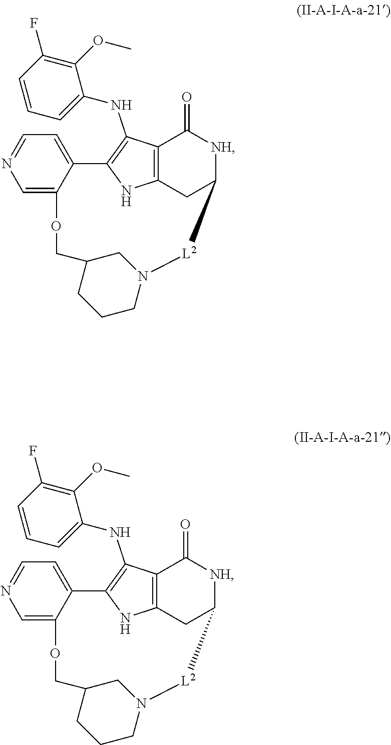

or a pharmaceutically acceptable salt, stereoisomer, solvate, prodrug, isotopic derivative, or tautomer thereof.

In some embodiments, the compound is of Formula (II-A-I-A-a-22):

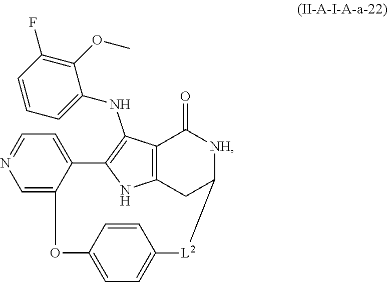

or a pharmaceutically acceptable salt, stereoisomer, solvate, prodrug, isotopic derivative, or tautomer thereof.

In some embodiments, the compound is of Formula (II-A-I-A-a-22′) or (II-A-I-A-a-22″):

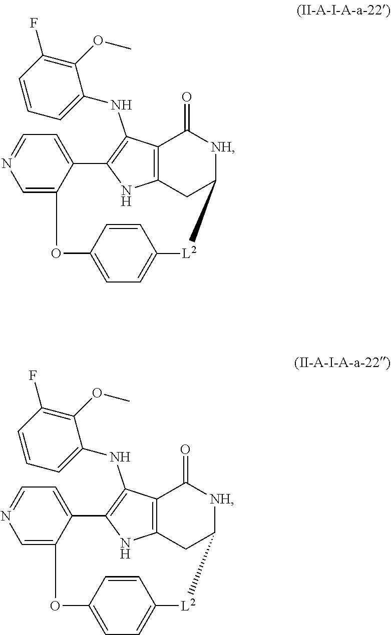

or a pharmaceutically acceptable salt, stereoisomer, solvate, prodrug, isotopic derivative, or tautomer thereof.

In some embodiments, the compound is of Formula (II-A-I-A-a-23):

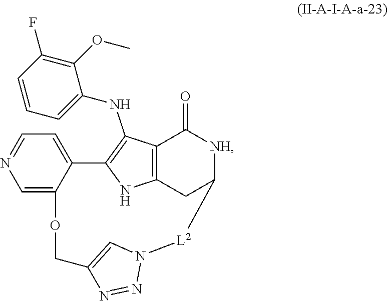

or a pharmaceutically acceptable salt, stereoisomer, solvate, prodrug, isotopic derivative, or tautomer thereof.

In some embodiments, the compound is of Formula (II-A-I-A-a-23′) or (II-A-I-A-a-23″):

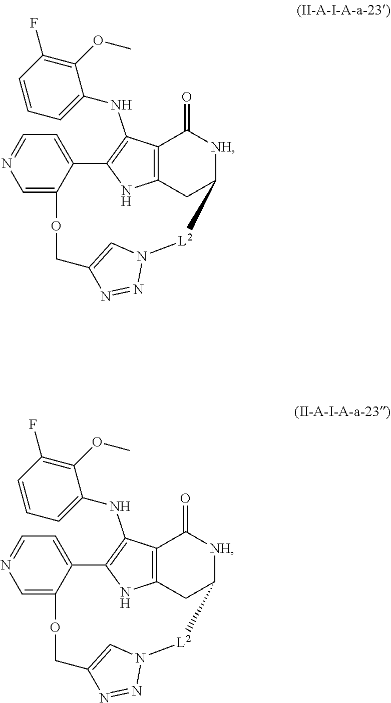

or a pharmaceutically acceptable salt, stereoisomer, solvate, prodrug, isotopic derivative, or tautomer thereof.

In some embodiments, the compound is of Formula (II-A-I-A-a-24):

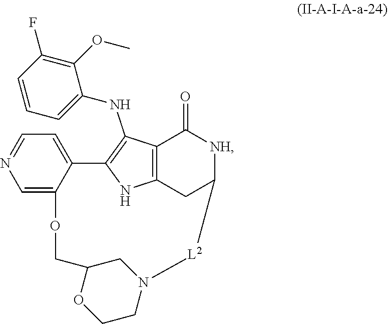

or a pharmaceutically acceptable salt, stereoisomer, solvate, prodrug, isotopic derivative, or tautomer thereof.

In some embodiments, the compound is of Formula (II-A-I-A-a-24′), (II-A-I-A-a-24″), (II-A-I-A-a-24″′), or (II-A-I-A-a-24″″):

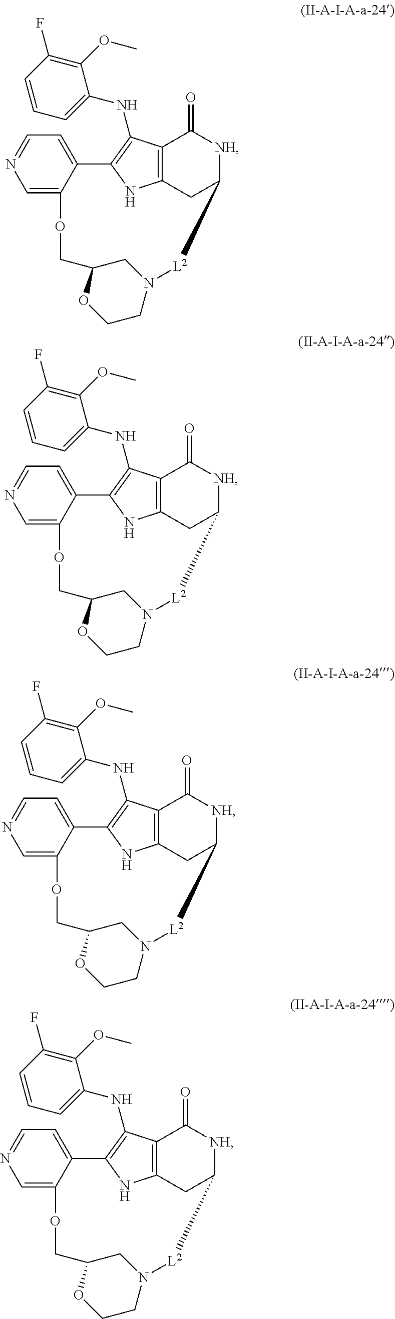

or a pharmaceutically acceptable salt, stereoisomer, solvate, prodrug, isotopic derivative, or tautomer thereof.

In some embodiments, the compound is of Formula (II-A-I-A-a-25):

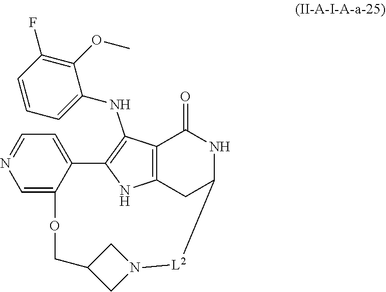

or a pharmaceutically acceptable salt, stereoisomer, solvate, prodrug, isotopic derivative, or tautomer thereof.

In some embodiments, the compound is of Formula (II-A-I-A-a-25′), (II-A-I-A-a-25″), (II-A-I-A-a-25″′), or(II-A-I—-″″):

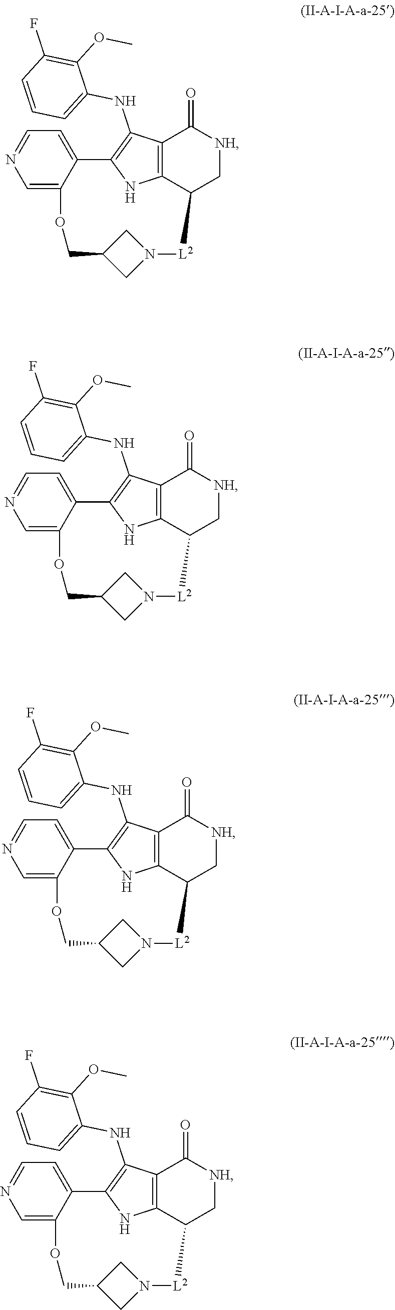

or a pharmaceutically acceptable salt, stereoisomer, solvate, prodrug, isotopic derivative, or tautomer thereof.

In some embodiments, the compound is of Formula (II-A-I-A-a-26):

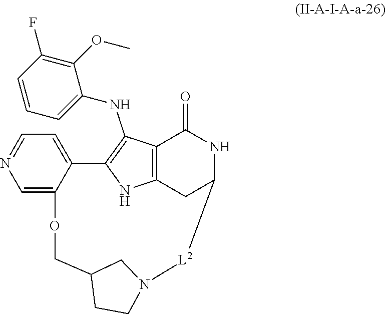

or a pharmaceutically acceptable salt, stereoisomer, solvate, prodrug, isotopic derivative, or tautomer thereof.

In some embodiments, the compound is of Formula (II-A-I-A-a-26′), (II-A-I-A-a-26″), (II—-A-T-A-a-26′″), or (II-A-I-A-a-26″″)

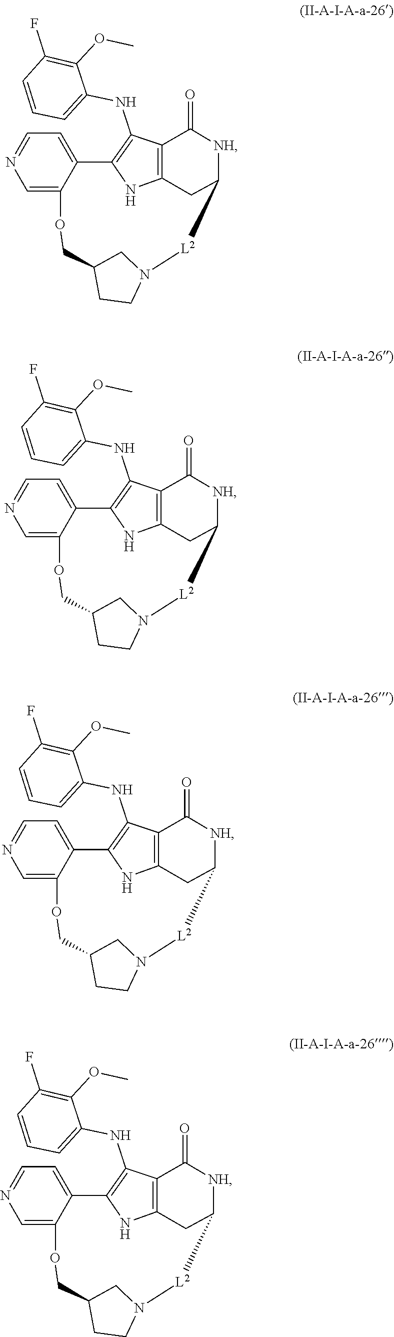

or a pharmaceutically acceptable salt, stereoisomer, solvate, prodrug, isotopic derivative, or tautomer thereof.

In some embodiments, the compound is of Formula (II-A-I-A-a-27):

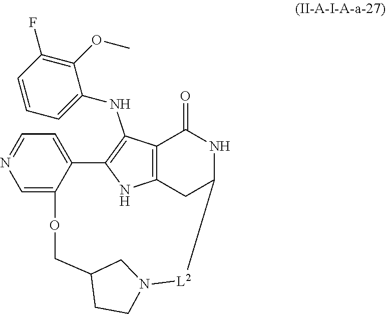

or a pharmaceutically acceptable salt, stereoisomer, solvate, prodrug, isotopic derivative, or tautomer thereof.

In some embodiments, the compound is of Formula (II-A-I-A-a-27′), (II-A-I-A-a-27″), (II-A-I-A-a-27′″), or (II-A-I-A-a-27″″):

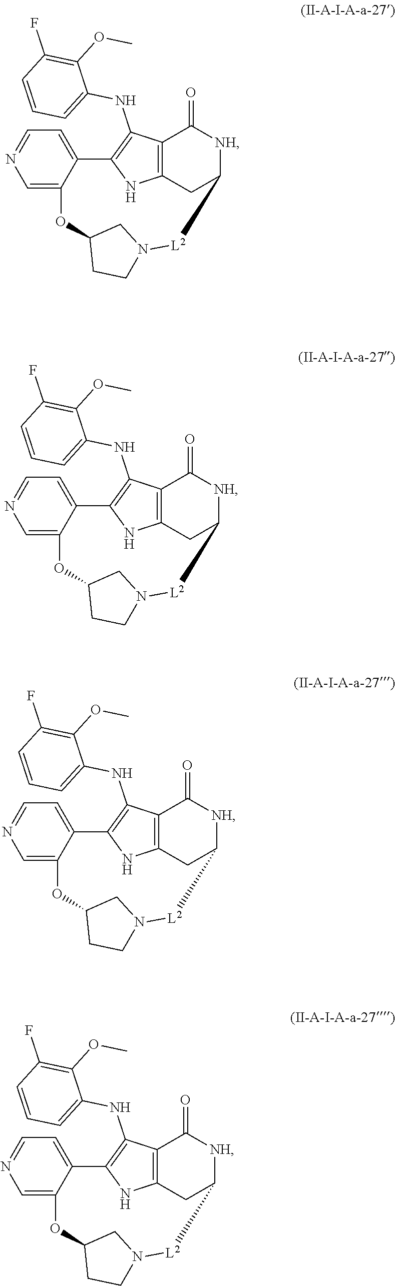

or a pharmaceutically acceptable salt, stereoisomer, solvate, prodrug, isotopic derivative, or tautomer thereof.

In some embodiments, the compound is of Formula (II-A-I-A-a-28):

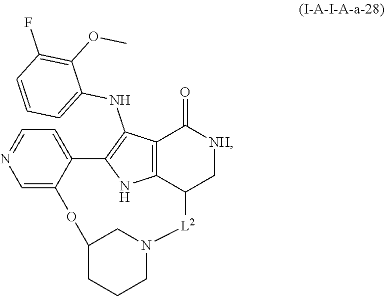

or a pharmaceutically acceptable salt, stereoisomer, solvate, prodrug, isotopic derivative, or tautomer thereof.

In some embodiments, the compound is of Formula (II-A-I-A-a-28′), (II-A-I-A-a-28″), (II-A-I-A-a-28″′), or (II-A-I-A-a-28″″):

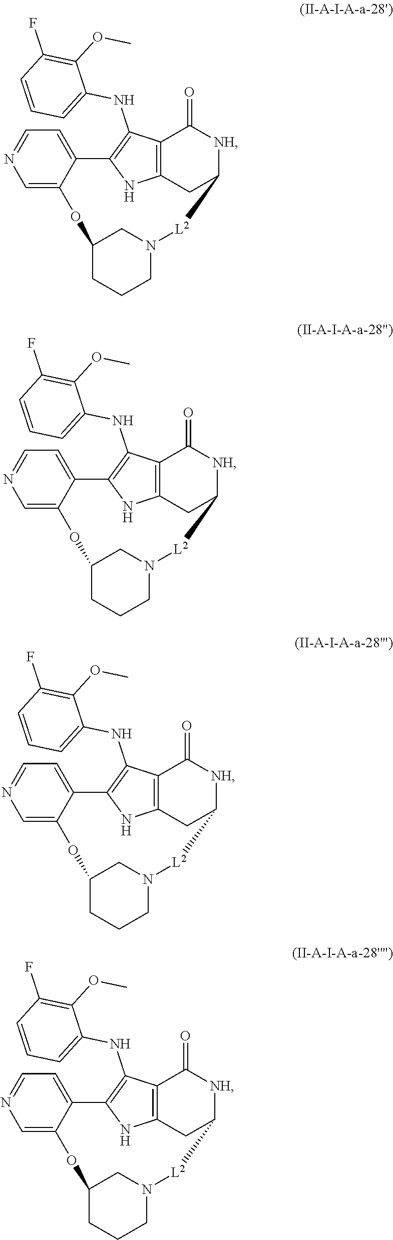

or a pharmaceutically acceptable salt, stereoisomer, solvate, prodrug, isotopic derivative, or tautomer thereof.

In some embodiments, the compound is of Formula (II-A-I-A-a-29):

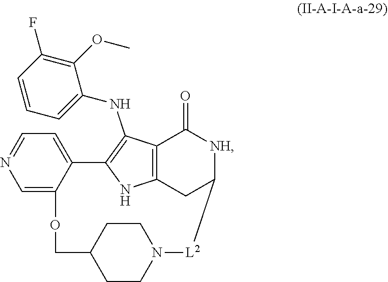

or a pharmaceutically acceptable salt, stereoisomer, solvate, prodrug, isotopic derivative, or tautomer thereof.

In some embodiments, the compound is of Formula (II-A-I-A-a-29′), (II-A-I-A-a-29″), (II-A-I-A-a-29′″), or (II-A-I-A-a-29″″):

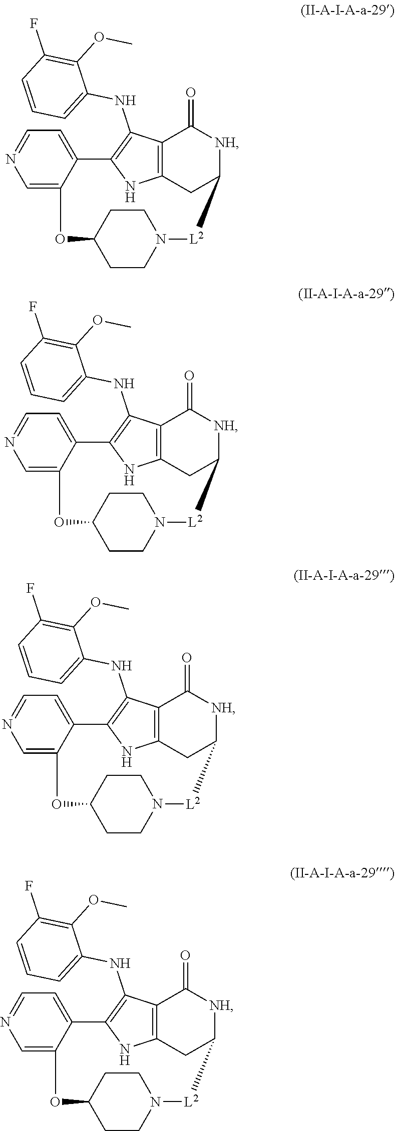

or a pharmaceutically acceptable salt, stereoisomer, solvate, prodrug, isotopic derivative, or tautomer thereof.

In some embodiments, the compound is of Formula (II-A-I-A-b-1):

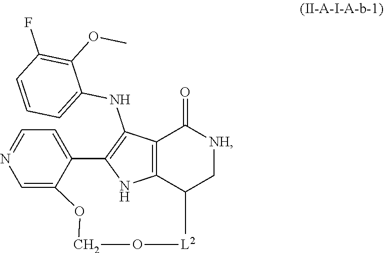

or a pharmaceutically acceptable salt, stereoisomer, solvate, prodrug, isotopic derivative, or tautomer thereof.

In some embodiments, the compound is of Formula (II-A-I-A-b-1′) or II-A-I-A-b-1″):

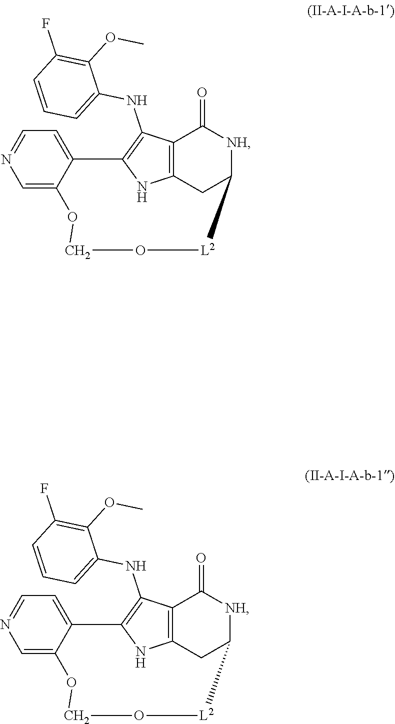

or a pharmaceutically acceptable salt, stereoisomer, solvate, prodrug, isotopic derivative, or tautomer thereof.

In some embodiments, the compound is of Formula (II-A-I-A-b-2):

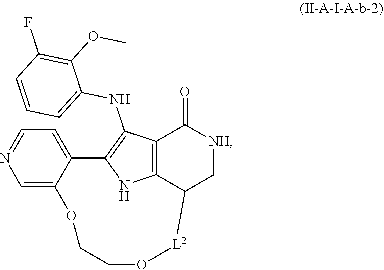

or a pharmaceutically acceptable salt, stereoisomer, solvate, prodrug, isotopic derivative, or tautomer thereof.

In some embodiments, the compound is of Formula (II-A-I-A-b-2′) or (II-A-I-A-b-2″):

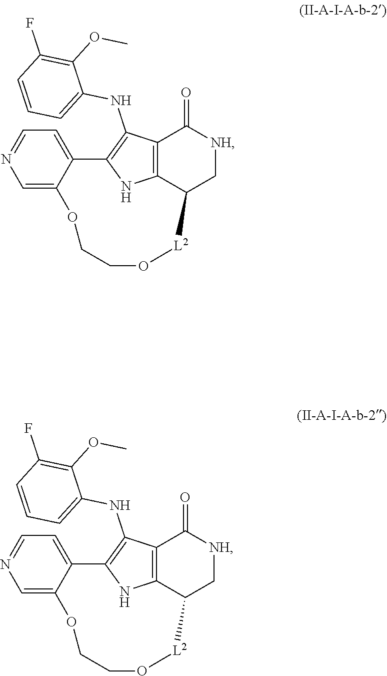

or a pharmaceutically acceptable salt, stereoisomer, solvate, prodrug, isotopic derivative, or tautomer thereof.

In some embodiments, the compound is of Formula (II-A-I-A-b-3):

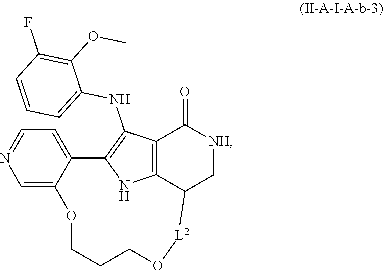

or a pharmaceutically acceptable salt, stereoisomer, solvate, prodrug, isotopic derivative, or tautomer thereof.

In some embodiments, the compound is of Formula (II-A-I-A-a-3′) or (II-A-I-A-a-3″):

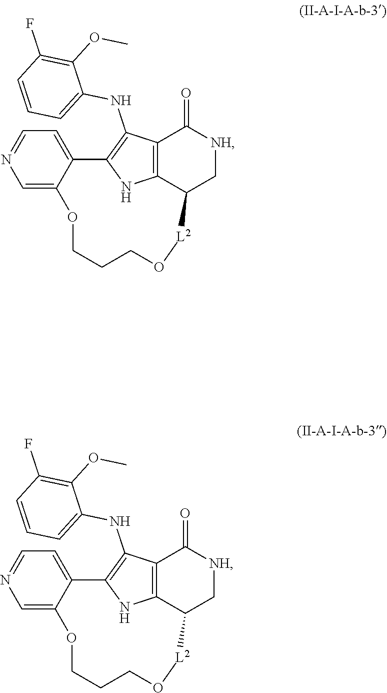

or a pharmaceutically acceptable salt, stereoisomer, solvate, prodrug, isotopic derivative, or tautomer thereof.

In some embodiments, the compound is of Formula (II-A-I-A-b-4):

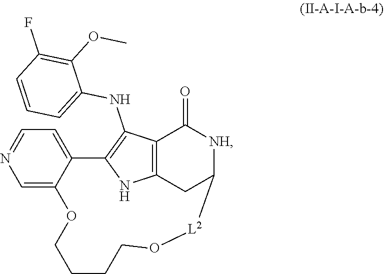

or a pharmaceutically acceptable salt, stereoisomer, solvate, prodrug, isotopic derivative, or tautomer thereof.

In some embodiments, the compound is of Formula (II-A-I-A-b-4′) or (II-A-I-A-b-4″):

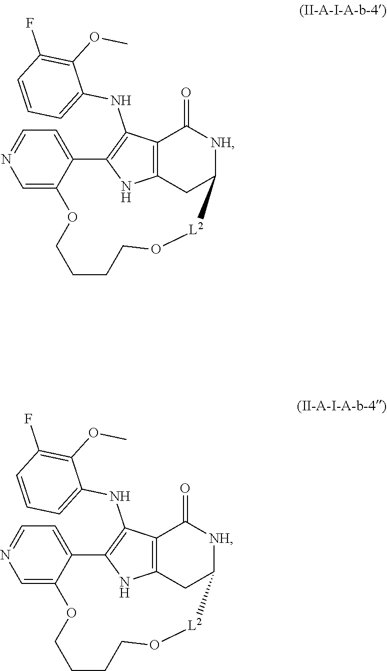

or a pharmaceutically acceptable salt, stereoisomer, solvate, prodrug, isotopic derivative, or tautomer thereof.

In some embodiments, the compound is of Formula (II-A-I-A-b-5):

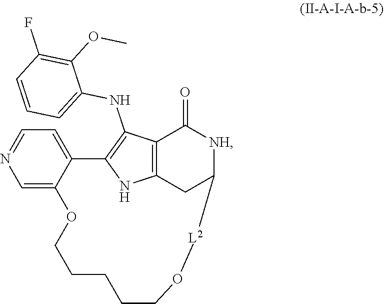

or a pharmaceutically acceptable salt, stereoisomer, solvate, prodrug, isotopic derivative, or tautomer thereof.

In some embodiments, the compound is of Formula (II-A-I-A-b-5′) or (II-A-I-A-b-5″):

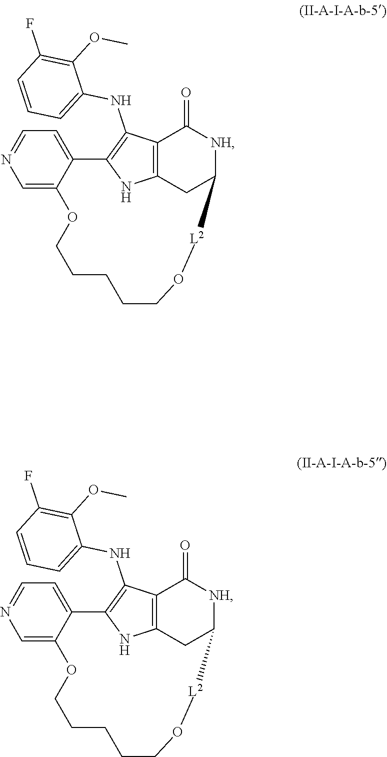

or a pharmaceutically acceptable salt, stereoisomer, solvate, prodrug, isotopic derivative, or tautomer thereof.

In some embodiments, the compound is of Formula (II-A-I-A-b-6):

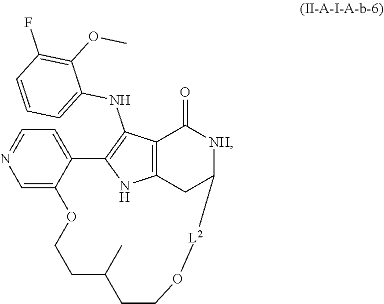

or a pharmaceutically acceptable salt, stereoisomer, solvate, prodrug, isotopic derivative, or tautomer thereof.

In some embodiments, the compound is of Formula (II-A-I-A-a-6′) or (II-A-I-A-a-6″):

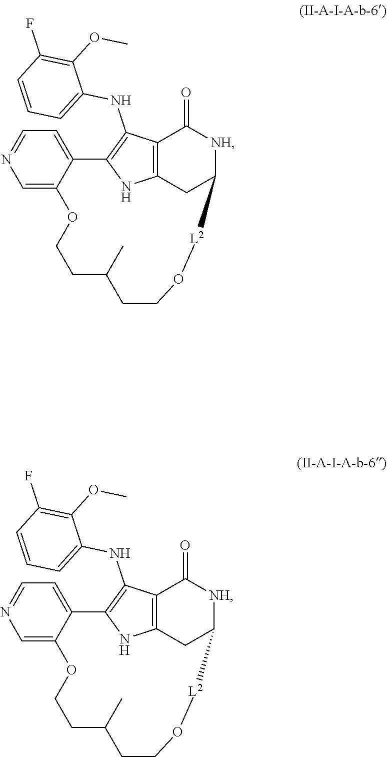

or a pharmaceutically acceptable salt, stereoisomer, solvate, prodrug, isotopic derivative, or tautomer thereof.

In some embodiments, the compound is of Formula (II-A-I-A-b-7):

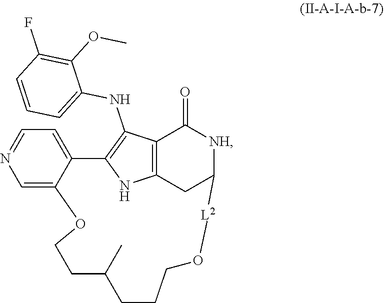

or a pharmaceutically acceptable salt, stereoisomer, solvate, prodrug, isotopic derivative, or tautomer thereof.

In some embodiments, the compound is of Formula (II-A-I-A-b-7′) or (II-A-I-A-b-7″):

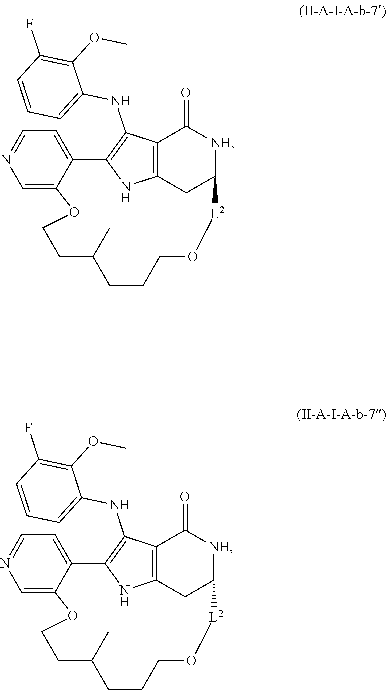

or a pharmaceutically acceptable salt, stereoisomer, solvate, prodrug, isotopic derivative, or tautomer thereof.

In some embodiments, the compound is of Formula (II-A-I-A-b-8):

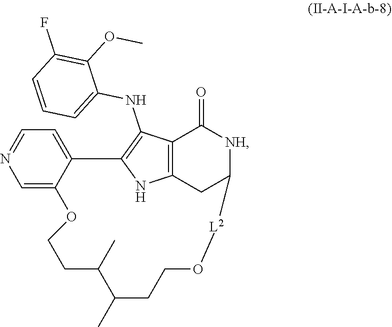

or a pharmaceutically acceptable salt, stereoisomer, solvate, prodrug, isotopic derivative, or tautomer thereof.

In some embodiments, the compound is of Formula (II-A-I-A-b-8′) or (II-A-I-A-b-8″):

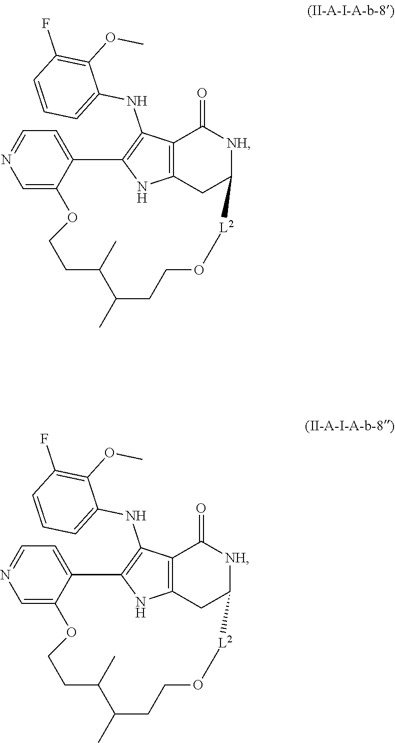

or a pharmaceutically acceptable salt, stereoisomer, solvate, prodrug, isotopic derivative, or tautomer thereof.

In some embodiments, the compound is of Formula (II-A-I-A-b-9):

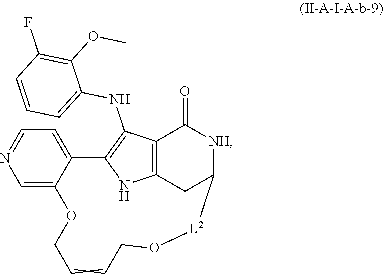

or a pharmaceutically acceptable salt, stereoisomer, solvate, prodrug, isotopic derivative, or tautomer thereof.

In some embodiments, the compound is of Formula (E-I-A-I-A-b-9) or (Z-I-A-I-A-b-9):

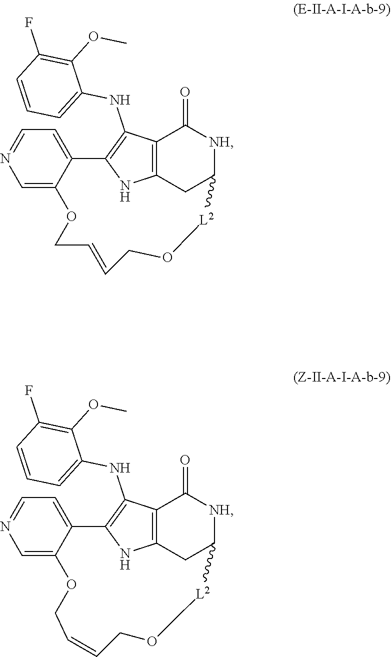

or a pharmaceutically acceptable salt, stereoisomer, solvate, prodrug, isotopic derivative, or tautomer thereof.

In some embodiments, the compound is of Formula (II-A-I-A-b-10):

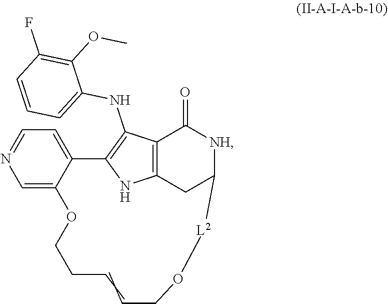

or a pharmaceutically acceptable salt, stereoisomer, solvate, prodrug, isotopic derivative, or tautomer thereof.

In some embodiments, the compound is of Formula (E-II-A-I-A-b-10) or (Z-II-A-I-A-b-10):

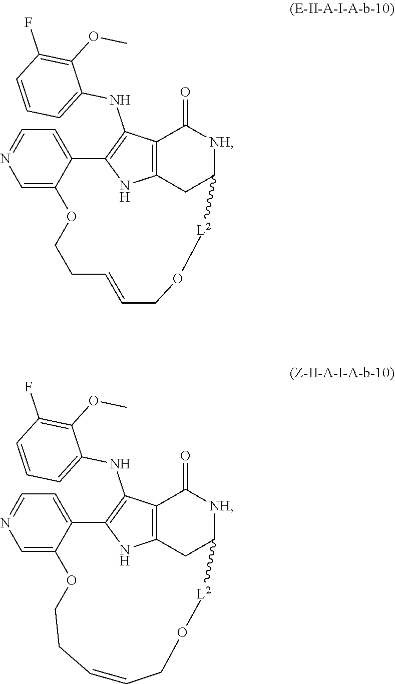

or a pharmaceutically acceptable salt, stereoisomer, solvate, prodrug, isotopic derivative, or tautomer thereof.

In some embodiments, the compound is of Formula (II-A-I-A-b-11):

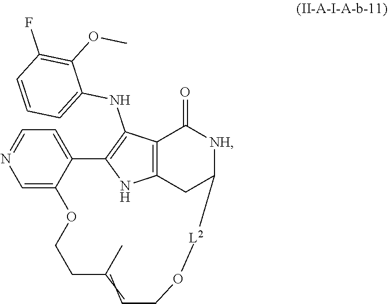

or a pharmaceutically acceptable salt, stereoisomer, solvate, prodrug, isotopic derivative, or tautomer thereof.

In some embodiments, the compound is of Formula (E-II-A-I-A-b-11) or (Z-II-A-I-A-b-11):

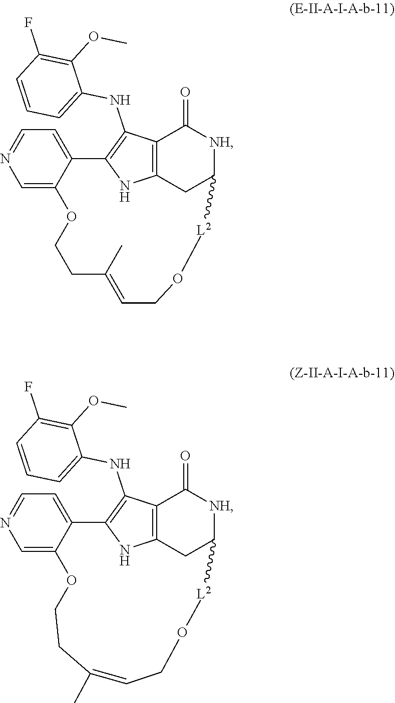

or a pharmaceutically acceptable salt, stereoisomer, solvate, prodrug, isotopic derivative, or tautomer thereof.

In some embodiments, the compound is of Formula (II-A-I-A-b-12):

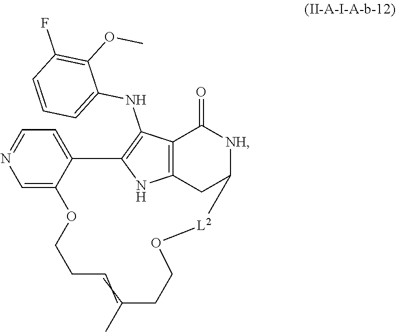

or a pharmaceutically acceptable salt, stereoisomer, solvate, prodrug, isotopic derivative, or tautomer thereof.

In some embodiments, the compound is of Formula (E-II-A-I-A-b-12) or (Z-II-A-I-A-b-12):

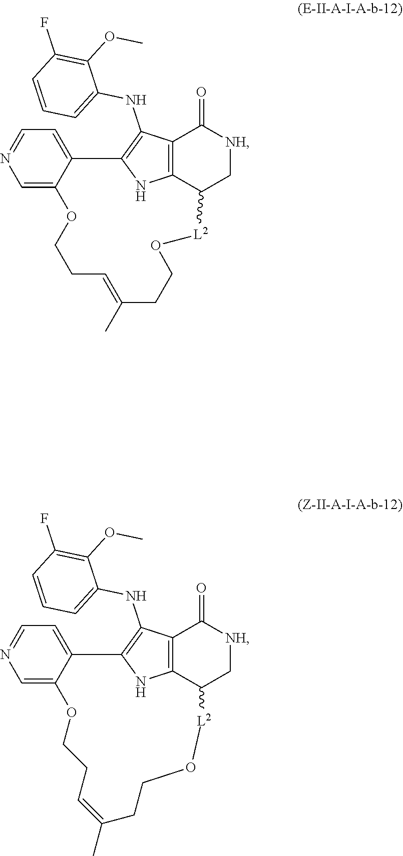

or a pharmaceutically acceptable salt, stereoisomer, solvate, prodrug, isotopic derivative, or tautomer thereof.

In some embodiments, the compound is of Formula (II-A-I-A-b-13):

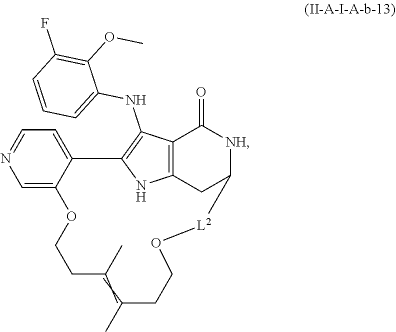

or a pharmaceutically acceptable salt, stereoisomer, solvate, prodrug, isotopic derivative, or tautomer thereof.

In some embodiments, the compound is of Formula (E-II-A-I-A-b-13) or (Z-II-A-I-A-b-13):

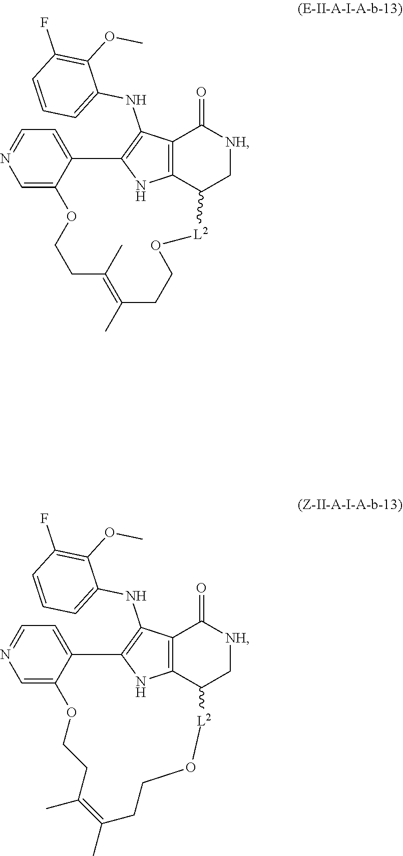

or a pharmaceutically acceptable salt, stereoisomer, solvate, prodrug, isotopic derivative, or tautomer thereof.

In some embodiments, the compound is of Formula (II-A-I-A-b-14):

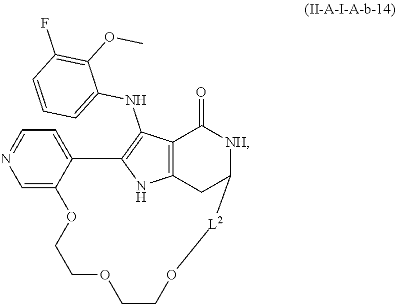

or a pharmaceutically acceptable salt, stereoisomer, solvate, prodrug, isotopic derivative, or tautomer thereof.

In some embodiments, the compound is of Formula (II-A-I-A-b-14′) or (II-A-I-A-b-14″):

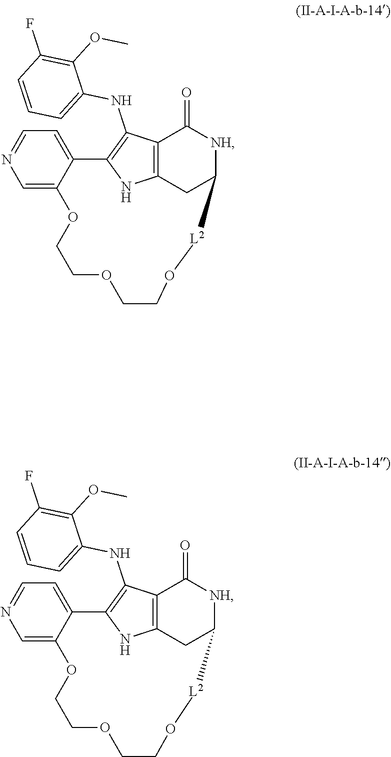

or a pharmaceutically acceptable salt, stereoisomer, solvate, prodrug, isotopic derivative, or tautomer thereof.

In some embodiments, the compound is of Formula (II-A-I-A-b-15):

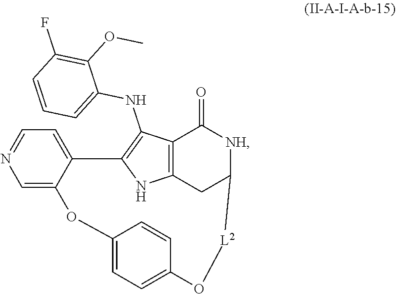

or a pharmaceutically acceptable salt, stereoisomer, solvate, prodrug, isotopic derivative, or tautomer thereof.

In some embodiments, the compound is of Formula (II-A-I-A-b-15′) or (II-A-I-A-b-15″):

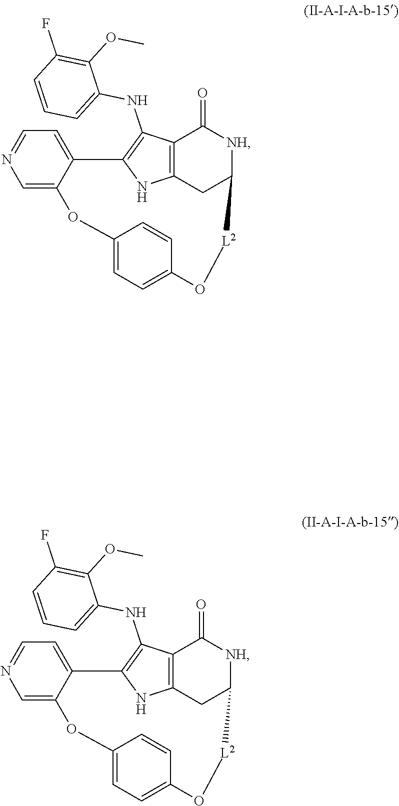

or a pharmaceutically acceptable salt, stereoisomer, solvate, prodrug, isotopic derivative, or tautomer thereof.

In some embodiments, the compound is of Formula (II-A-I-A-b-16):

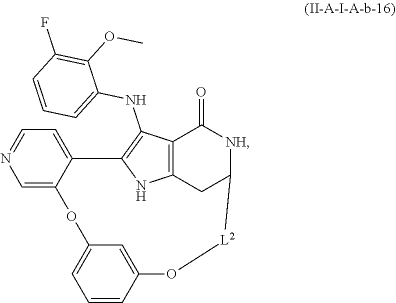

or a pharmaceutically acceptable salt, stereoisomer, solvate, prodrug, isotopic derivative, or tautomer thereof.

In some embodiments, the compound is of Formula (II-A-I-A-b-16′) or (II-A-I-A-b-16″):

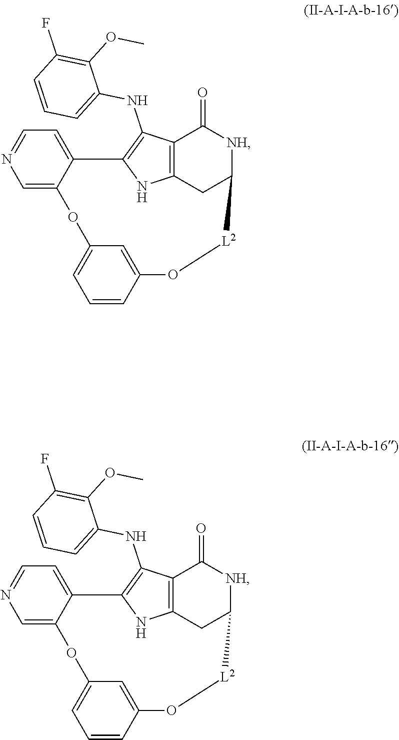

or a pharmaceutically acceptable salt, stereoisomer, solvate, prodrug, isotopic derivative, or tautomer thereof.

In some embodiments, the compound is of Formula (II-A-I-A-b-17):

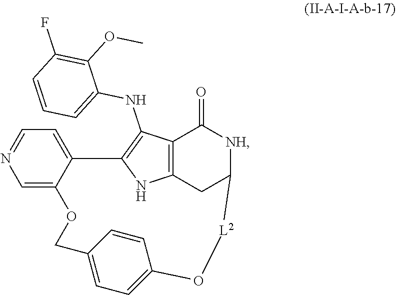

or a pharmaceutically acceptable salt, stereoisomer, solvate, prodrug, isotopic derivative, or tautomer thereof.

In some embodiments, the compound is of Formula (II-A-I-A-b-17′) or (II-A-I-A-b-17″):

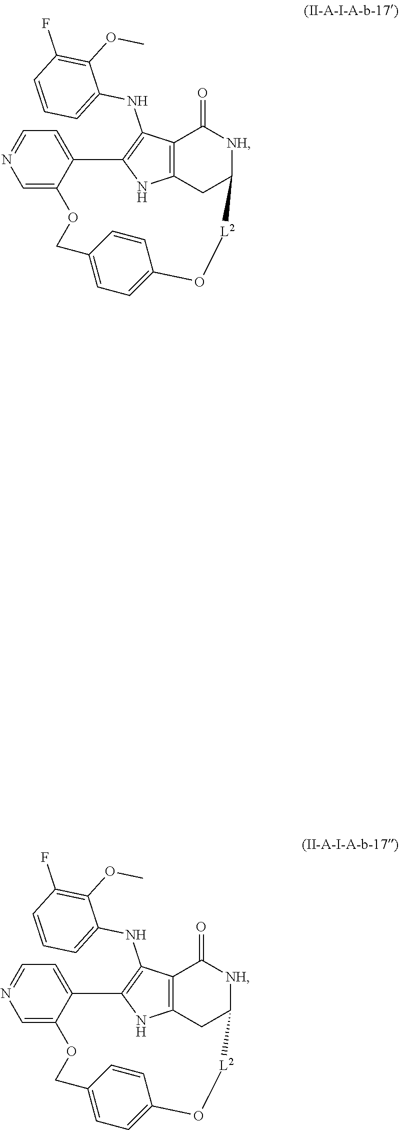

or a pharmaceutically acceptable salt, stereoisomer, solvate, prodrug, isotopic derivative, or tautomer thereof.

In some embodiments, the compound is of Formula (II-A-I-A-b-18):

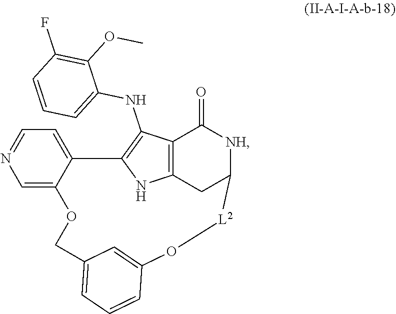

or a pharmaceutically acceptable salt, stereoisomer, solvate, prodrug, isotopic derivative, or tautomer thereof.

In some embodiments, the compound is of Formula (II-A-I-A-b-18′) or (II-A-I-A-b-18″):

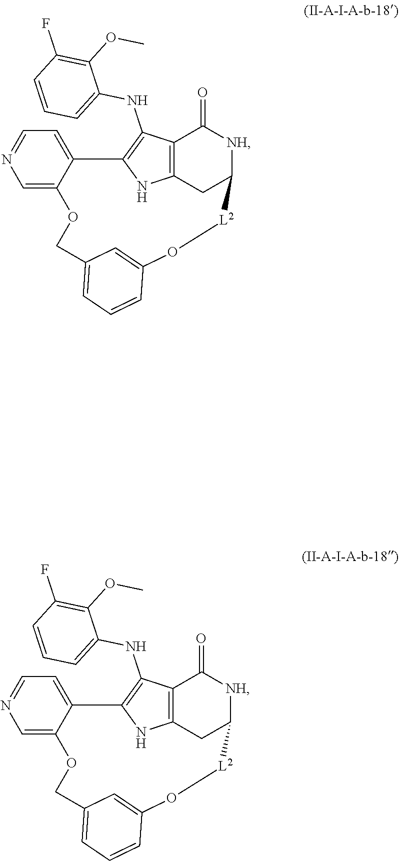

or a pharmaceutically acceptable salt, stereoisomer, solvate, prodrug, isotopic derivative, or tautomer thereof.

In some embodiments, the compound is of Formula (II-A-I-A-b-19):

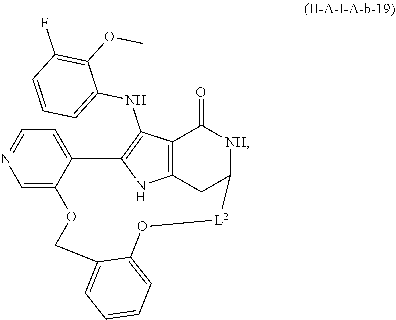

or a pharmaceutically acceptable salt, stereoisomer, solvate, prodrug, isotopic derivative, or tautomer thereof.

In some embodiments, the compound is of Formula (II-A-I-A-b-19′) or (II-A-I-A-b-19″):

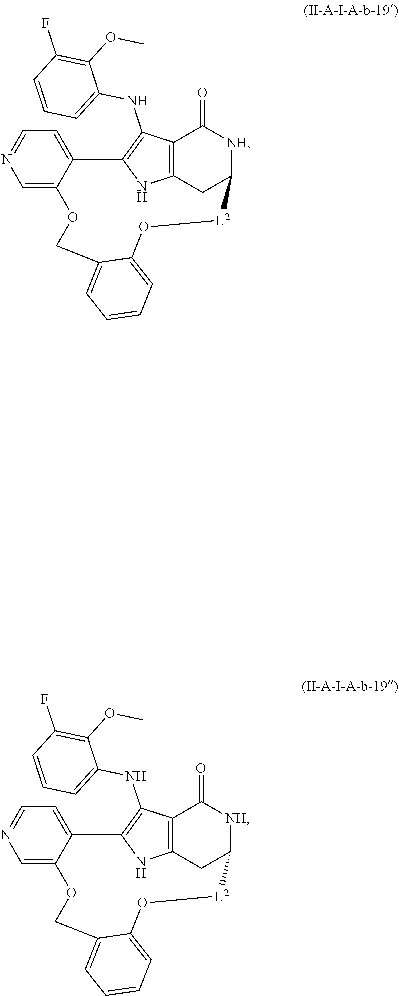

or a pharmaceutically acceptable salt, stereoisomer, solvate, prodrug, isotopic derivative, or tautomer thereof.

In some embodiments, the compound is of Formula (II-A-I-A-c-1):

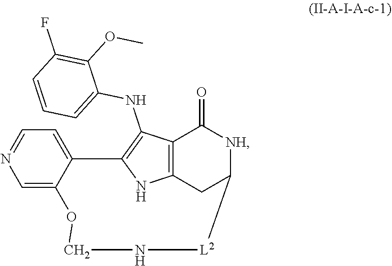

or a pharmaceutically acceptable salt, stereoisomer, solvate, prodrug, isotopic derivative, or tautomer thereof.

In some embodiments, the compound is of Formula (II-A-I-A-c-1′) or (II-A-I-A-c-1″):

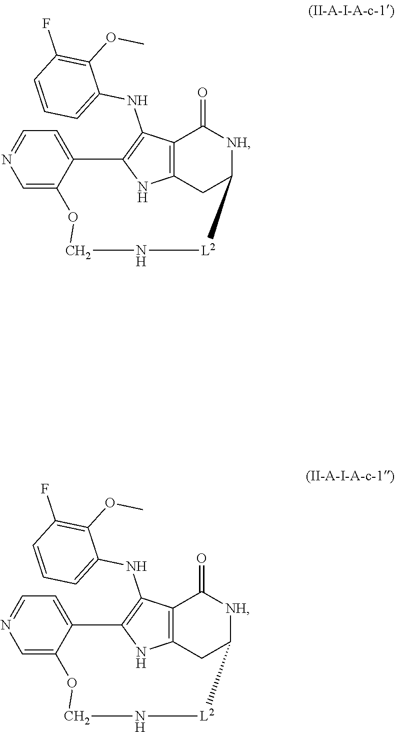

or a pharmaceutically acceptable salt, stereoisomer, solvate, prodrug, isotopic derivative, or tautomer thereof.

In some embodiments, the compound is of Formula (II-A-I-A-c-2):

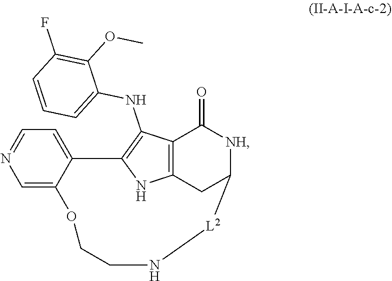

or a pharmaceutically acceptable salt, stereoisomer, solvate, prodrug, isotopic derivative, or tautomer thereof.

In some embodiments, the compound is of Formula (II-A-I-A-c-2′) or (II-A-I-A-c-2″):

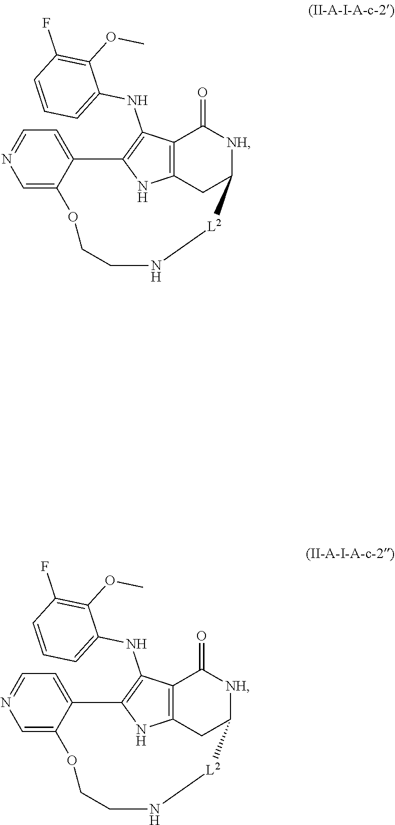

or a pharmaceutically acceptable salt, stereoisomer, solvate, prodrug, isotopic derivative, or tautomer thereof.

In some embodiments, the compound is of Formula (II-A-I-A-c-3):

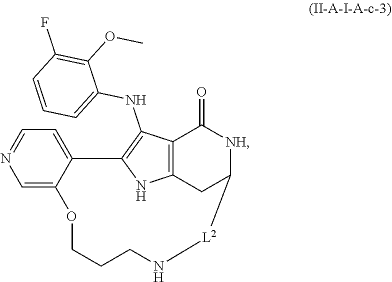

or a pharmaceutically acceptable salt, stereoisomer, solvate, prodrug, isotopic derivative, or tautomer thereof.

In some embodiments, the compound is of Formula (II-A-I-A-c-3′) or (II-A-I-A-c-3″):

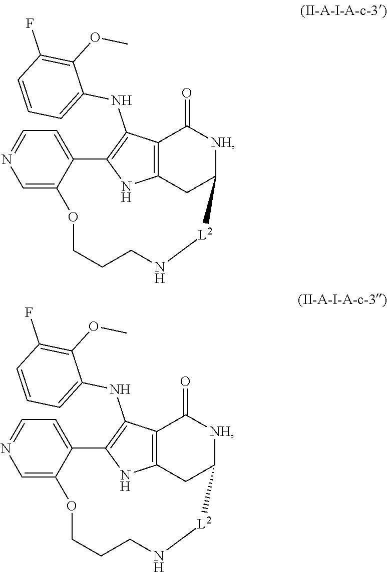

or a pharmaceutically acceptable salt, stereoisomer, solvate, prodrug, isotopic derivative, or tautomer thereof.

In some embodiments, the compound is of Formula (II-A-I-A-c-4):

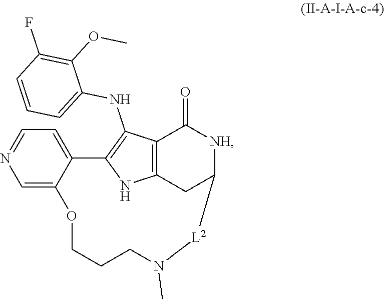

or a pharmaceutically acceptable salt, stereoisomer, solvate, prodrug, isotopic derivative, or tautomer thereof.

In some embodiments, the compound is of Formula (II-A-I-A-c-4′) or (II-A-I-A-c-4″):

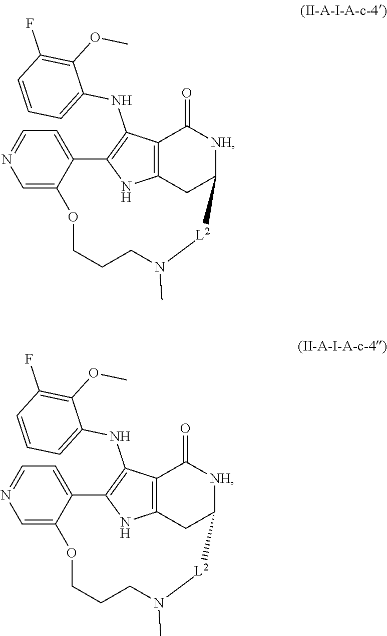

or a pharmaceutically acceptable salt, stereoisomer, solvate, prodrug, isotopic derivative, or tautomer thereof.

In some embodiments, the compound is of Formula (II-A-I-A-c-5):

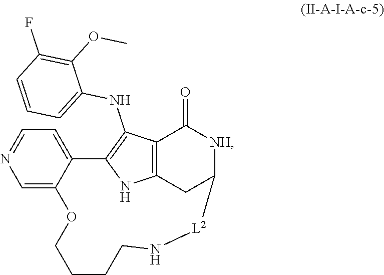

or a pharmaceutically acceptable salt, stereoisomer, solvate, prodrug, isotopic derivative, or tautomer thereof.

In some embodiments, the compound is of Formula (II-A-I-A-c-5′) or (II-A-I-A-c-5″):

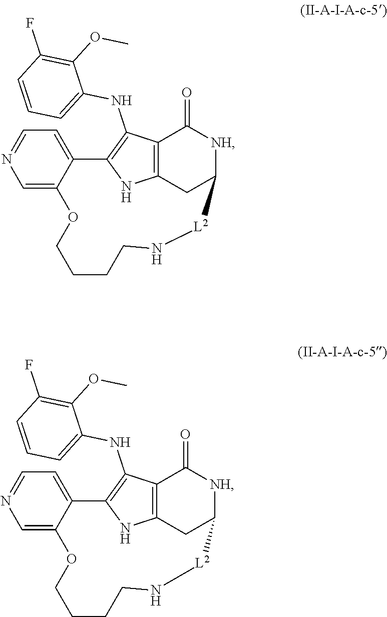

or a pharmaceutically acceptable salt, stereoisomer, solvate, prodrug, isotopic derivative, or tautomer thereof.

In some embodiments, the compound is of Formula (II-A-I-A-c-6):

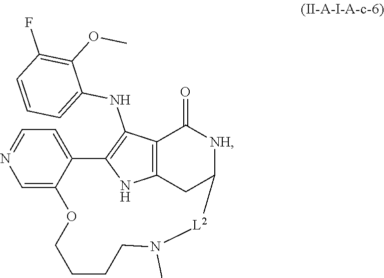

or a pharmaceutically acceptable salt, stereoisomer, solvate, prodrug, isotopic derivative, or tautomer thereof.

In some embodiments, the compound is of Formula (II-A-I-A-c-6′) or II-A-I-A-c-6″

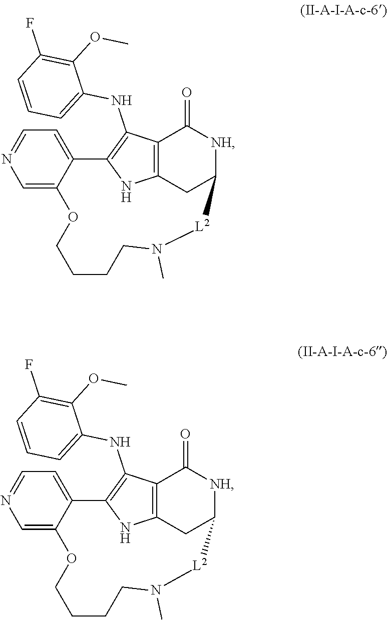

or a pharmaceutically acceptable salt, stereoisomer, solvate, prodrug, isotopic derivative, or tautomer thereof

In some embodiments, the compound is of Formula (II-A-I-A-c-7):

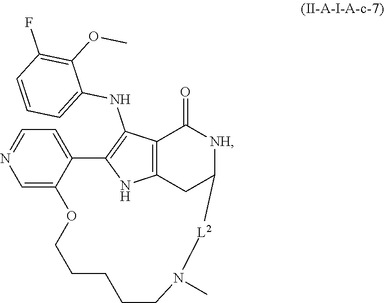

or a pharmaceutically acceptable salt, stereoisomer, solvate, prodrug, isotopic derivative, or tautomer thereof.

In some embodiments, the compound is of Formula (II-A-I-A-c-7′) or (II-A-I-A-c-7″):

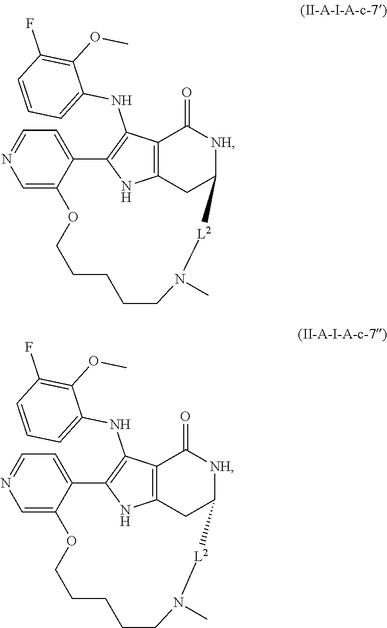

or a pharmaceutically acceptable salt, stereoisomer, solvate, prodrug, isotopic derivative, or tautomer thereof.

In some embodiments, the compound is of Formula (II-A-I-A-d-1):

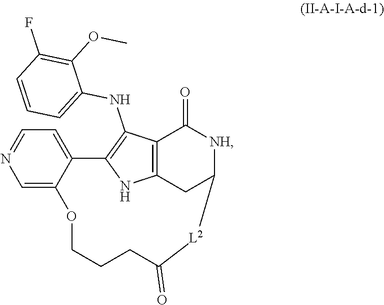

or a pharmaceutically acceptable salt, stereoisomer, solvate, prodrug, isotopic derivative, or tautomer thereof.

In some embodiments, the compound is of Formula (II-A-I-A-d-1′) or (II-A-I-A-d-1″):

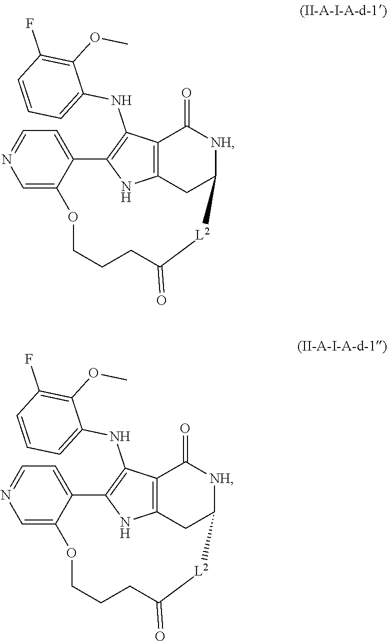

or a pharmaceutically acceptable salt, stereoisomer, solvate, prodrug, isotopic derivative, or tautomer thereof.

In some embodiments, the compound is of Formula (II-A-I-A-d-2):

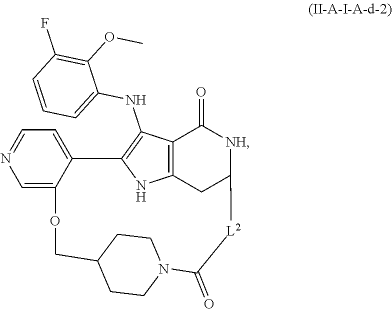

or a pharmaceutically acceptable salt, stereoisomer, solvate, prodrug, isotopic derivative, or tautomer thereof.

In some embodiments, the compound is of Formula (II-A-I-A-d-2′) or (II-A-1-A-d-2′):

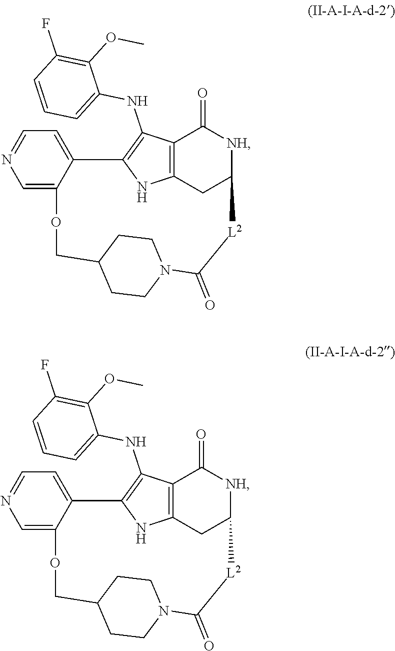

or a pharmaceutically acceptable salt, stereoisomer, solvate, prodrug, isotopic derivative, or tautomer thereof.

In some embodiments, the compound is of Formula (II-A-I-A-d-3):

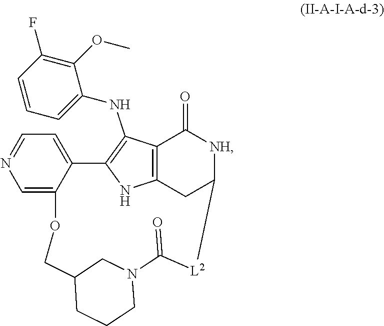

or a pharmaceutically acceptable salt, stereoisomer, solvate, prodrug, isotopic derivative, or tautomer thereof.

In some embodiments, the compound is of Formula (II-A-I-A-d-3′) or (II-A-I-A-d-3″):

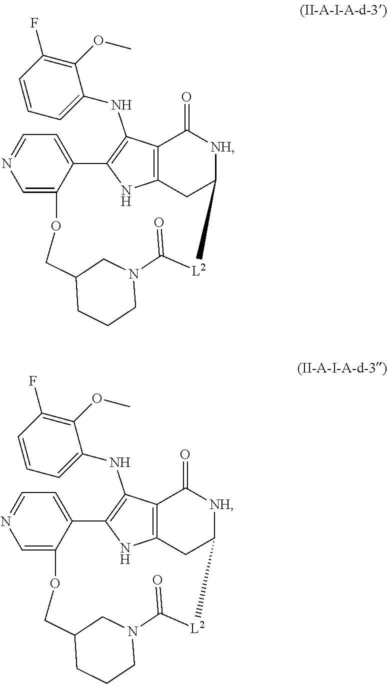

or a pharmaceutically acceptable salt, stereoisomer, solvate, pro rug, isotopic derivative, or tautomer thereof.

In some embodiments, the compound is of Formula (II-A-I-A-d-4):

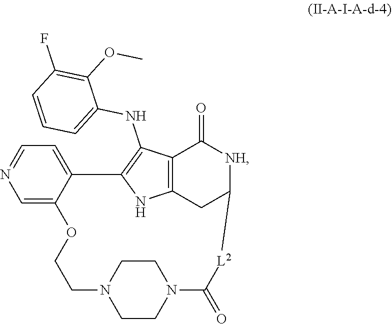

or a pharmaceutically acceptable salt, stereoisomer, solvate, prodrug, isotopic derivative, or tautomer thereof.

In some embodiments, the compound is of Formula (II-A-I-A-d-4′) or (II-A-I-A-d-4″):

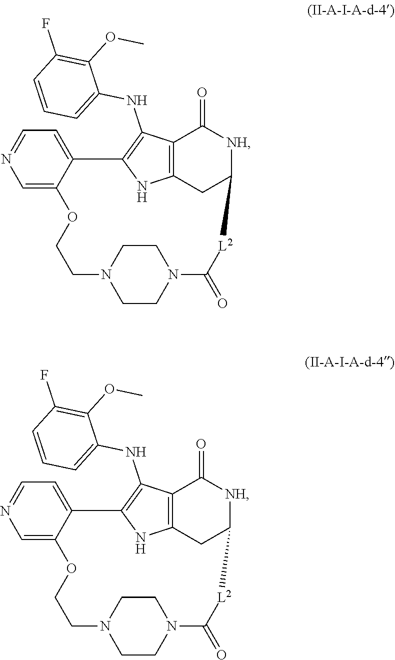

or a pharmaceutically acceptable salt, stereoisomer, solvate, prodrug, isotopic derivative, or tautomer thereof.

In some embodiments, the compound is of Formula (II-A-I-A-d-5):

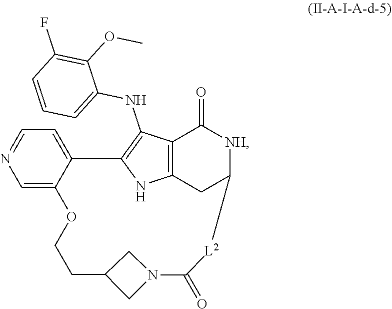

or a pharmaceutically acceptable salt, stereoisomer, solvate, prodrug, isotopic derivative, or tautomer thereof.

In some embodiments, the compound is of Formula (II-A-I-A-d-5′) or (II-A-I-A-d-5″):

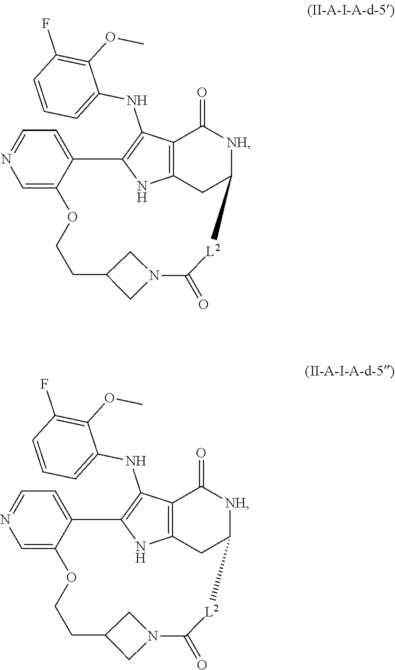

or a pharmaceutically acceptable salt, stereoisomer, solvate, prodrug, isotopic derivative, or tautomer thereof.

In some embodiments, the compound is of Formula (II-A-I-A-e-1):

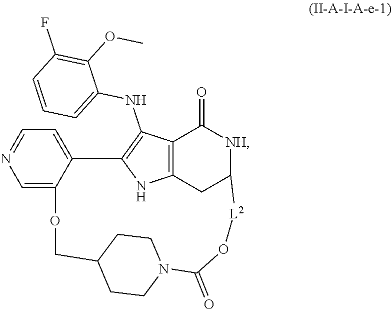

or a pharmaceutically acceptable salt, stereoisomer, solvate, prodrug, isotopic derivative, or tautomer thereof.

In some embodiments, the compound is of Formula (II-A-I-A-e-1′) or (II-A-I-A-e-1″):

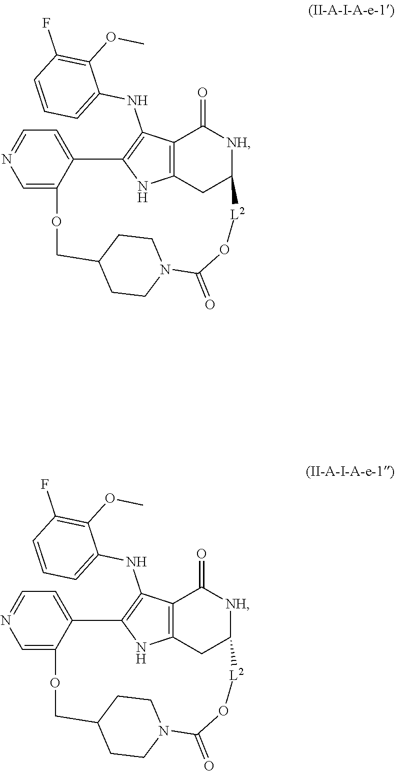

or a pharmaceutically acceptable salt, stereoisomer, solvate, prodrug, isotopic derivative, or tautomer thereof.

In some embodiments, the compound is of Formula (II-A-I-A-e-2):

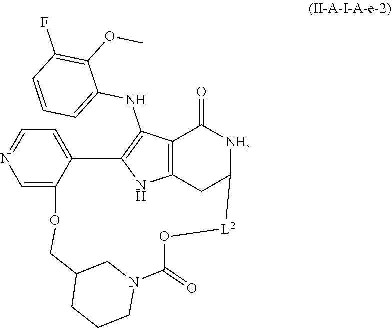

or a pharmaceutically acceptable salt, stereoisomer, solvate, prodrug, isotopic derivative, or tautomer thereof.

In some embodiments, the compound is of Formula (II-A-I-A-e-2′) or (II-A-I-A-e-2″):

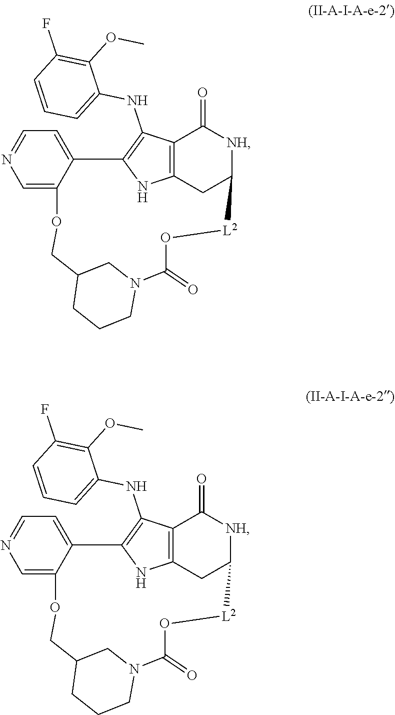

or a pharmaceutically acceptable salt, stereoisomer, solvate, prodrug, isotopic derivative, or tautomer thereof.

In some embodiments, the compound is of Formula (II-A-I-A-f-1):

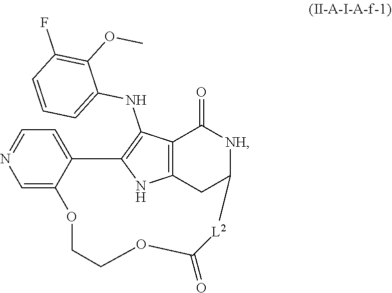

or a pharmaceutically acceptable salt, stereoisomer, solvate, prodrug, isotopic derivative, or tautomer thereof.

In some embodiments, the compound is of Formula (II-A-I-A-f-1′) or (II-A-I-A-f-2″):

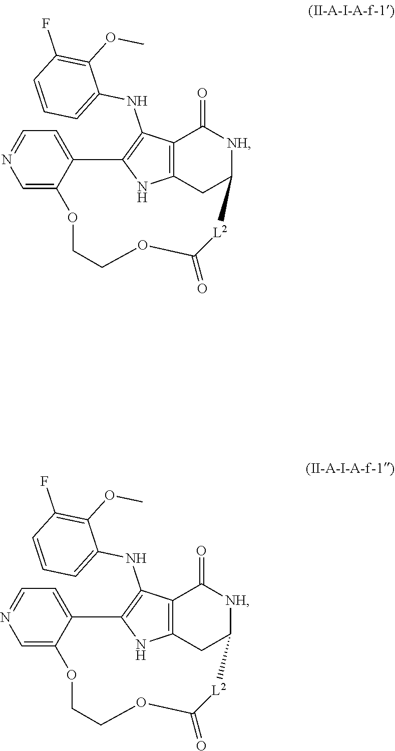

or a pharmaceutically acceptable salt, stereoisomer, solvate, prodrug, isotopic derivative, or tautomer thereof.

In some embodiments, the compound is of Formula (II-A-I-A-g-1):

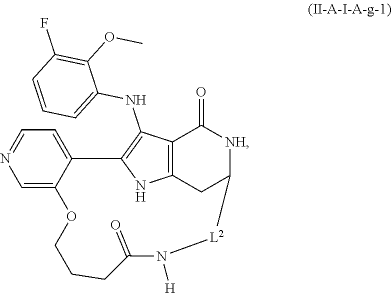

or a pharmaceutically acceptable salt, stereoisomer, solvate, prodrug, isotopic derivative, or tautomer thereof.

In some embodiments, the compound is of Formula (II-A-I-A-g-1′) or (II-A-I-A-g-1″):

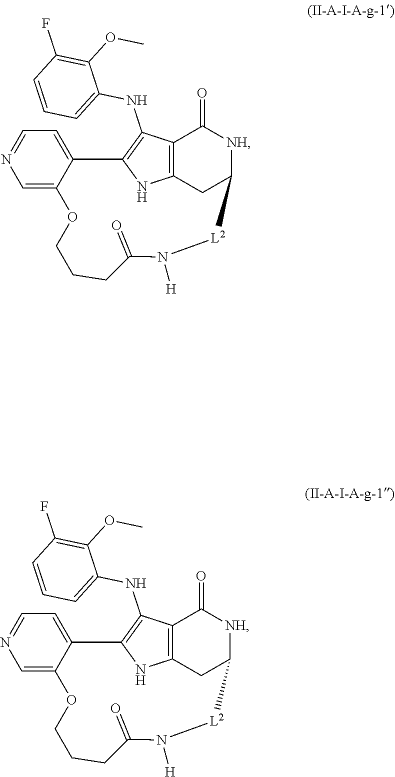

or a pharmaceutically acceptable salt, stereoisomer, solvate, prodrug, isotopic derivative, or tautomer thereof.

In some embodiments, the compound is of Formula (II-A-I-A-g-2):

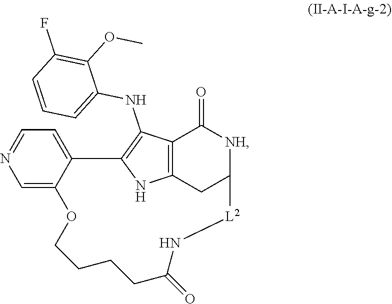

or a pharmaceutically acceptable salt, stereoisomer, solvate, prodrug, isotopic derivative, or tautomer thereof.

In some embodiments, the compound is of Formula (II-A-I-A-g-2′) or (II-A-I-A-g-2″):

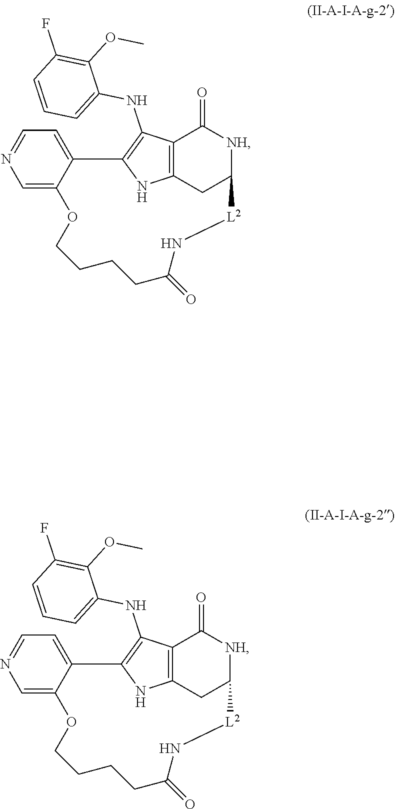

or a pharmaceutically acceptable salt, stereoisomer, solvate, prodrug, isotopic derivative, or tautomer thereof.

In some embodiments, the compound is of Formula (II-A-I-A-h-1):

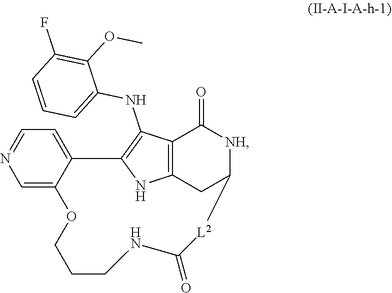

or a pharmaceutically acceptable salt, stereoisomer, solvate, prodrug, isotopic derivative, or tautomer thereof.

In some embodiments, the compound is of Formula (I-A-I-A-h-1′) or (I-A-I-A-h-1″):

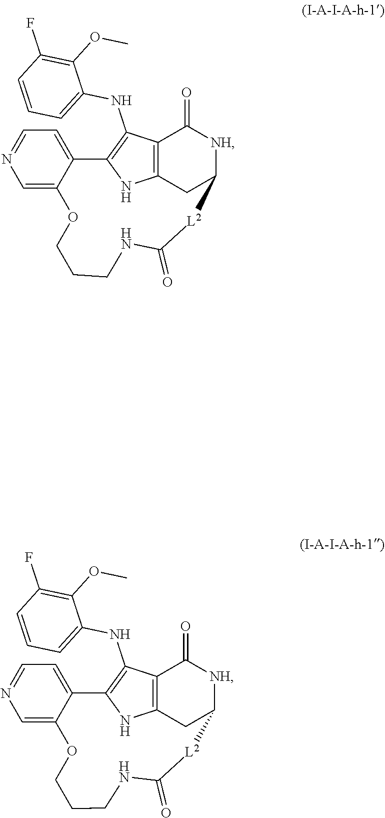

or a pharmaceutically acceptable salt, stereoisomer, solvate, prodrug, isotopic derivative, or tautomer thereof.

In some embodiments, the compound is of Formula (II-A-I-A-h-2):

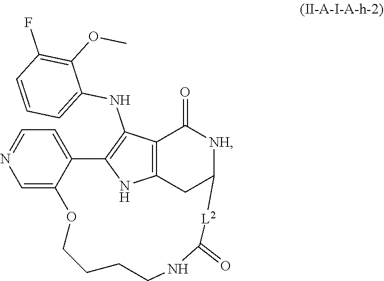

or a pharmaceutically acceptable salt, stereoisomer, solvate, prodrug, isotopic derivative, or tautomer thereof.

In some embodiments, the compound is of Formula (II-A-I-A-h-2′) or (II-A-I-A-h-2″):

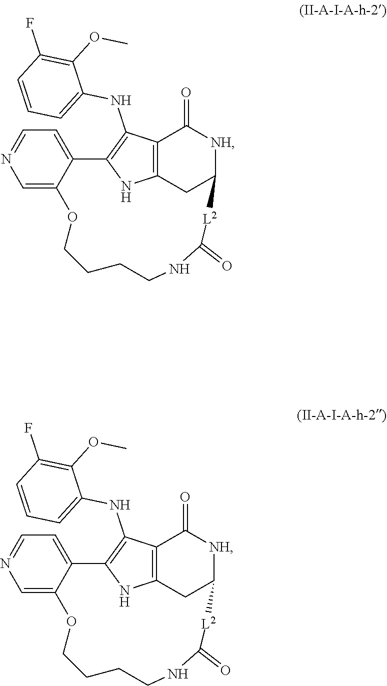

or a pharmaceutically acceptable salt, stereoisomer, solvate, prodrug, isotopic derivative, or tautomer thereof.

In some embodiments, the compound is of Formula (II-A-I-A-100):

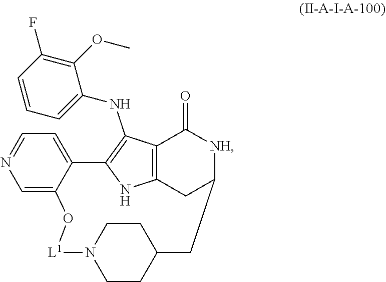

or a pharmaceutically acceptable salt, stereoisomer, solvate, prodrug, isotopic derivative, or tautomer thereof.

In some embodiments, the compound is of Formula (II-A-I-A-101):

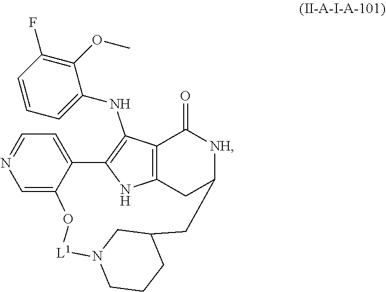

or a pharmaceutically acceptable salt, stereoisomer, solvate, prodrug, isotopic derivative, or tautomer thereof.

In some embodiments, the compound is of Formula (II-A-I-A-102):

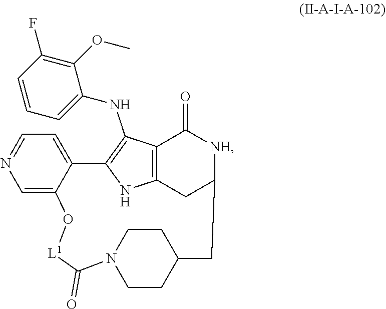

or a pharmaceutically acceptable salt, stereoisomer, solvate, prodrug, isotopic derivative, or tautomer thereof.

In some embodiments, the compound is of Formula (II-A-I-A-103):

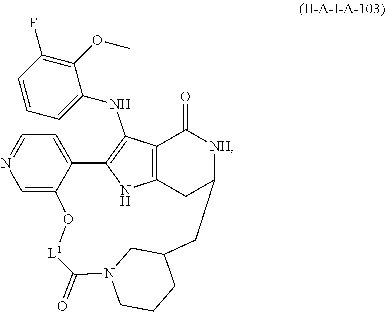

or a pharmaceutically acceptable salt, stereoisomer, solvate, prodrug, isotopic derivative, or tautomer thereof.

In some embodiments, the compound is of Formula (II-A-I-A-104):

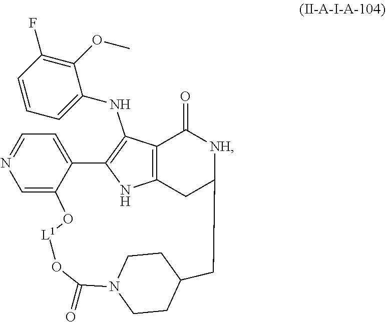

or a pharmaceutically acceptable salt, stereoisomer, solvate, prodrug, isotopic derivative, or tautomer thereof.

In some embodiments, the compound is of Formula (II-A-I-A-105):

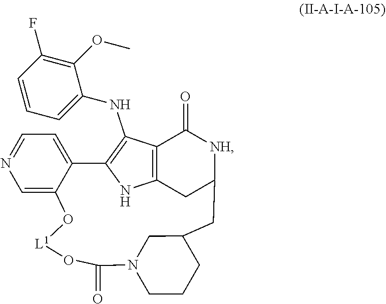

or a pharmaceutically acceptable salt, stereoisomer, solvate, prodrug, isotopic derivative, or tautomer thereof.

In some embodiments, the compound is of Formula (II-A-I-A-106):

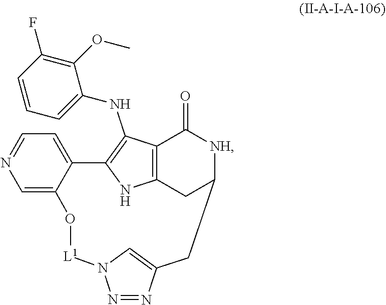

or a pharmaceutically acceptable salt, stereoisomer, solvate, prodrug, isotopic derivative, or tautomer thereof.

In some embodiments, the compound is of Formula (II-A-I-A-107):

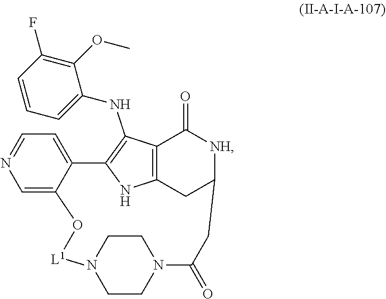

or a pharmaceutically acceptable salt, stereoisomer, solvate, prodrug, isotopic derivative, or tautomer thereof.

In some embodiments, the compound is of Formula (II-A-I-A-108):

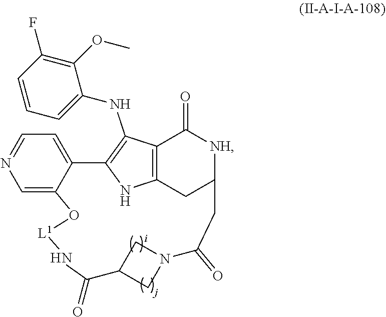

or a pharmaceutically acceptable salt, stereoisomer, solvate, prodrug, isotopic derivative, or tautomer thereof, wherein i is an integer selected from 0, 1, 2, and 3; j is an integer selected from 1, 2, 3, 4 and 5; and all other variables are as defined herein.

In some embodiments, the compound is of Formula (II-A-I-A-109):

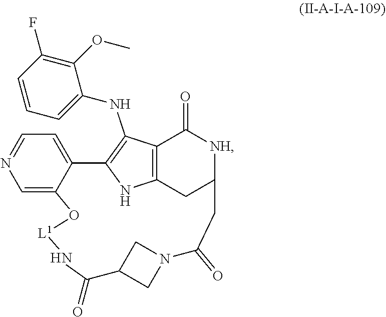

or a pharmaceutically acceptable salt, stereoisomer, solvate, prodrug, isotopic derivative, or tautomer thereof.

In some embodiments, the compound is of Formula (II-A-I-A-110):

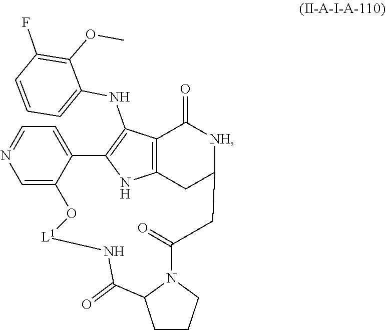

or a pharmaceutically acceptable salt, stereoisomer, solvate, prodrug, isotopic derivative, or tautomer thereof.

In some embodiments, the compound is of Formula (II-A-I-A-111):

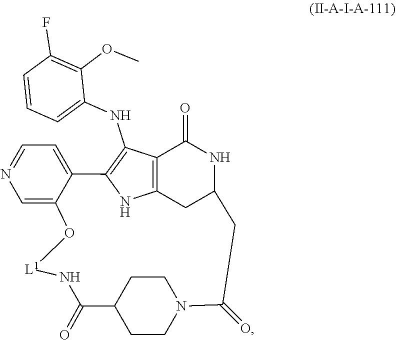

or a pharmaceutically acceptable salt, stereoisomer, solvate, prodrug, isotopic derivative, or tautomer thereof.

In some embodiments, the compound is of Formula (II-A-I-B-a-1):

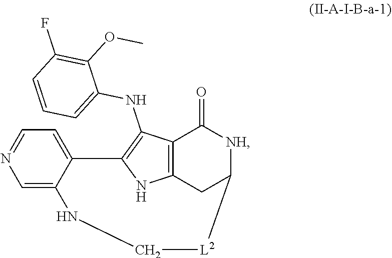

or a pharmaceutically acceptable salt, stereoisomer, solvate, prodrug, isotopic derivative, or tautomer thereof.

In some embodiments, the compound is of Formula (II-A-I-B-a-2):

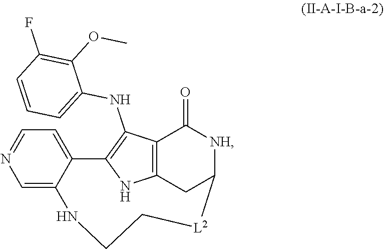

or a pharmaceutically acceptable salt, stereoisomer, solvate, prodrug, isotopic derivative, or tautomer thereof.

In some embodiments, the compound is of Formula (II-A-I-B-a-3):

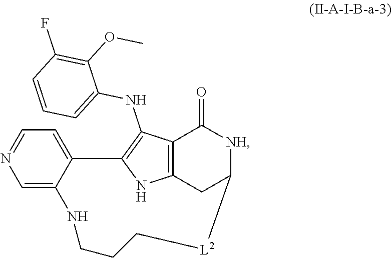

or a pharmaceutically acceptable salt, stereoisomer, solvate, prodrug, isotopic derivative, or tautomer thereof.

In some embodiments, the compound is of Formula (II-A-I-B-a-4):

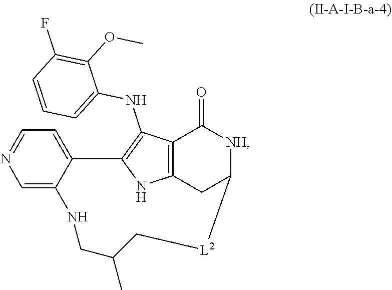

or a pharmaceutically acceptable salt, stereoisomer, solvate, prodrug, isotopic derivative, or tautomer thereof.

In some embodiments, the compound is of Formula (II-A-I-B-a-5):

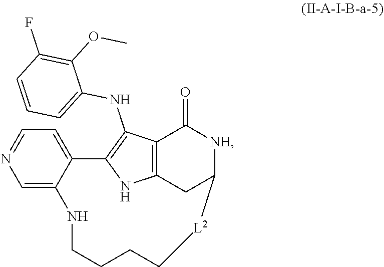

or a pharmaceutically acceptable salt, stereoisomer, solvate, prodrug, isotopic derivative, or tautomer thereof.

In some embodiments, the compound is of Formula (II-A-I-B-a-6):

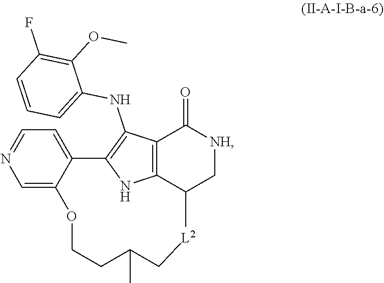

or a pharmaceutically acceptable salt, stereoisomer, solvate, prodrug, isotopic derivative, or tautomer thereof.

In some embodiments, the compound is of Formula (II-A-I-B-a-7):

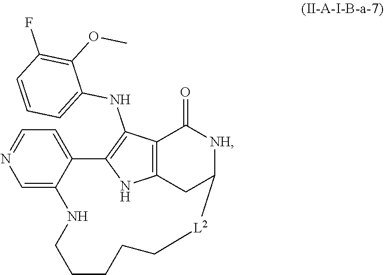

or a pharmaceutically acceptable salt, stereoisomer, solvate, prodrug, isotopic derivative, or tautomer thereof.

In some embodiments, the compound is of Formula (II-A-I-B-a-8):

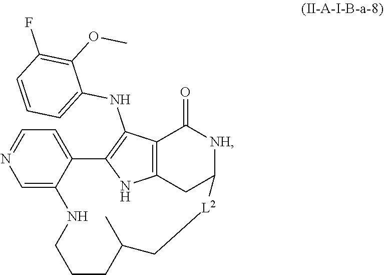

or a pharmaceutically acceptable salt, stereoisomer, solvate, prodrug, isotopic derivative, or tautomer thereof.

In some embodiments, the compound is of Formula (II-A-I-B-a-9):

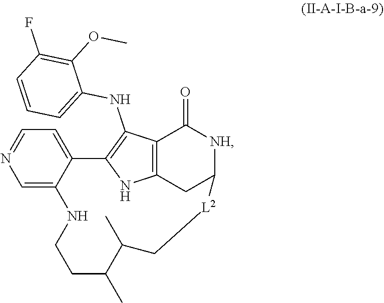

or a pharmaceutically acceptable salt, stereoisomer, solvate, prodrug, isotopic derivative, or tautomer thereof.

In some embodiments, the compound is of Formula (II-A-I-B-a-10):

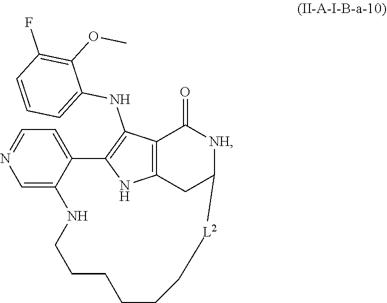

or a pharmaceutically acceptable salt, stereoisomer, solvate, prodrug, isotopic derivative, or tautomer thereof.

In some embodiments, the compound is of Formula (II-A-I-B-a-11):

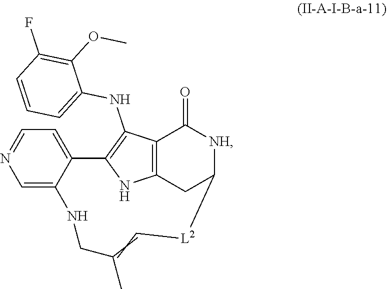

or a pharmaceutically acceptable salt, stereoisomer, solvate, prodrug, isotopic derivative, or tautomer thereof.

In some embodiments, the compound is of Formula (II-A-I-B-a-12):

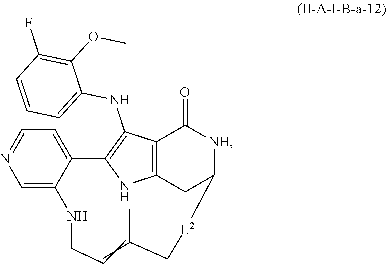

or a pharmaceutically acceptable salt, stereoisomer, solvate, prodrug, isotopic derivative, or tautomer thereof.

In some embodiments, the compound is of Formula (II-A-I-B-a-13):

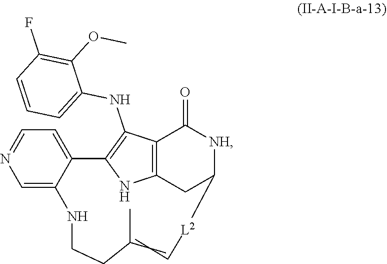

or a pharmaceutically acceptable salt, stereoisomer, solvate, prodrug, isotopic derivative, or tautomer thereof.

In some embodiments, the compound is of Formula (II-A-I-B-a-14):

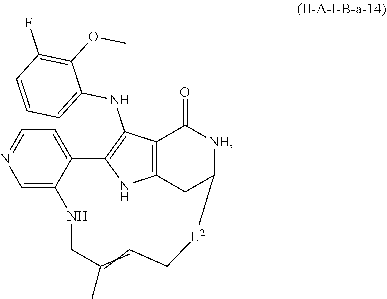

or a pharmaceutically acceptable salt, stereoisomer, solvate, prodrug, isotopic derivative, or tautomer thereof.

In some embodiments, the compound is of Formula (II-A-I-B-a-15):

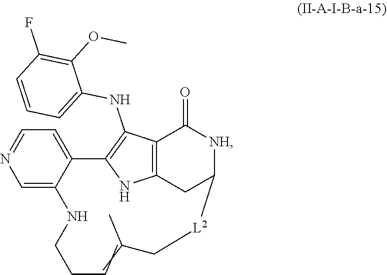

or a pharmaceutically acceptable salt, stereoisomer, solvate, prodrug, isotopic derivative, or tautomer thereof.

In some embodiments, the compound is of Formula (II-A-I-B-a-16):

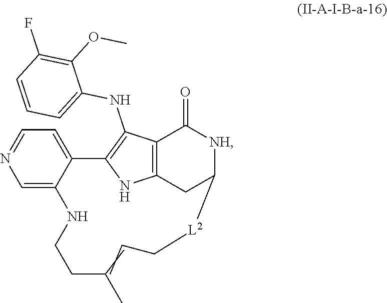

or a pharmaceutically acceptable salt, stereoisomer, solvate, prodrug, isotopic derivative, or tautomer thereof.

In some embodiments, the compound is of Formula (II-A-I-B-a-17):

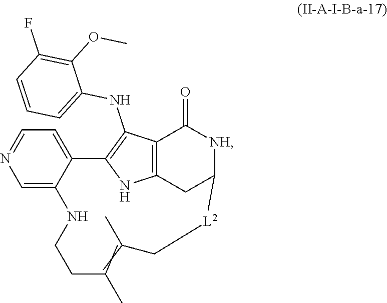

or a pharmaceutically acceptable salt, stereoisomer, solvate, prodrug, isotopic derivative, or tautomer thereof.

In some embodiments, the compound is of Formula (II-A-I-B-a-18):

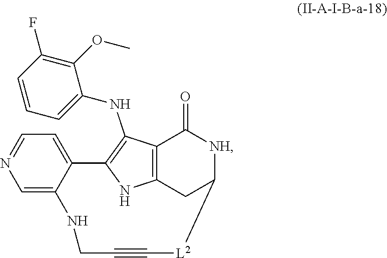

or a pharmaceutically acceptable salt, stereoisomer, solvate, prodrug, isotopic derivative, or tautomer thereof.

In some embodiments, the compound is of Formula (II-A-I-B-a-19):

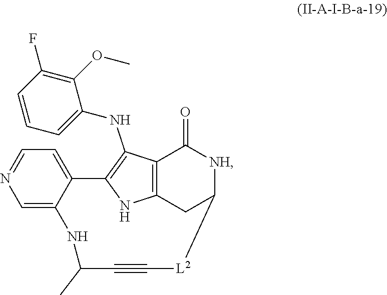

or a pharmaceutically acceptable salt, stereoisomer, solvate, prodrug, isotopic derivative, or tautomer thereof.

In some embodiments, the compound is of Formula (II-A-I-B-a-20):

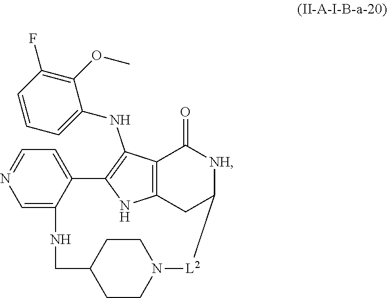

or a pharmaceutically acceptable salt, stereoisomer, solvate, prodrug, isotopic derivative, or tautomer thereof.

In some embodiments, the compound is of Formula (II-A-I-B-a-21):

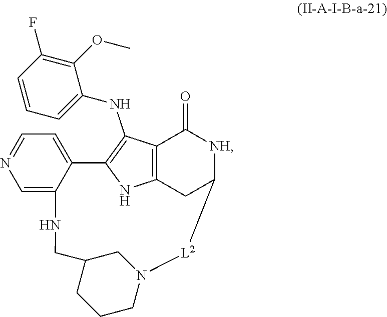

or a pharmaceutically acceptable salt, stereoisomer, solvate, prodrug, isotopic derivative, or tautomer thereof.

In some embodiments, the compound is of Formula (II-A-I-B-a-22):

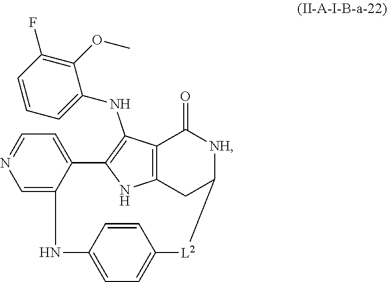

or a pharmaceutically acceptable salt, stereoisomer, solvate, prodrug, isotopic derivative, or tautomer thereof.

In some embodiments, the compound is of Formula (II-A-I-B-a-23):

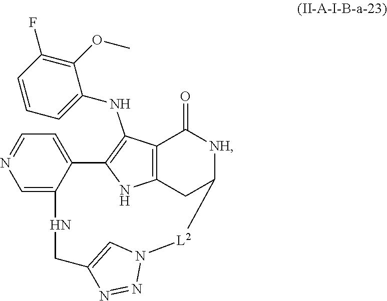

or a pharmaceutically acceptable salt, stereoisomer, solvate, prodrug, isotopic derivative, or tautomer thereof.

In some embodiments, the compound is of Formula (II-A-I-B-a-24):

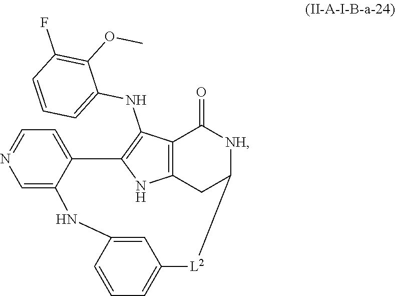

or a pharmaceutically acceptable salt, stereoisomer, solvate, prodrug, isotopic derivative, or tautomer thereof.

In some embodiments, the compound is of Formula (II-A-I-B-b-1):

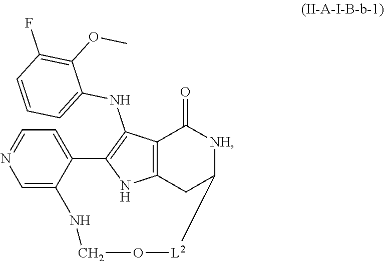

or a pharmaceutically acceptable salt, stereoisomer, solvate, prodrug, isotopic derivative, or tautomer thereof.

In some embodiments, the compound is of Formula (II-A-I-B-b-2):

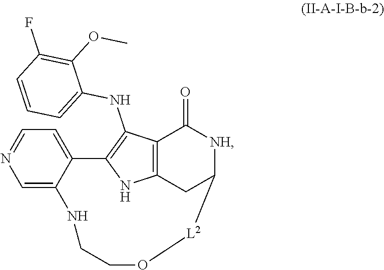

or a pharmaceutically acceptable salt, stereoisomer, solvate, prodrug, isotopic derivative, or tautomer thereof.

In some embodiments, the compound is of Formula (II-A-I-B-b-3):

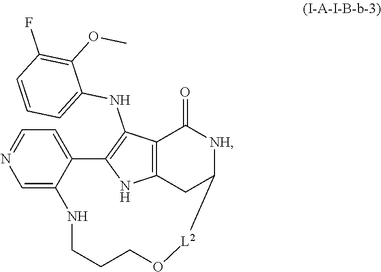

or a pharmaceutically acceptable salt, stereoisomer, solvate, prodrug, isotopic derivative, or tautomer thereof.

In some embodiments, the compound is of Formula (II-A-I-B-b-4):

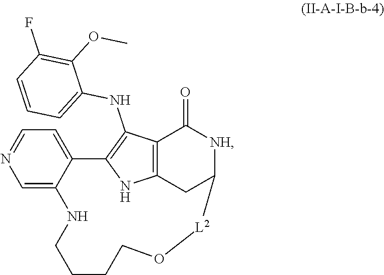

or a pharmaceutically acceptable salt, stereoisomer, solvate, prodrug, isotopic derivative, or tautomer thereof.

In some embodiments, the compound is of Formula (II-A-I-B-b-5):

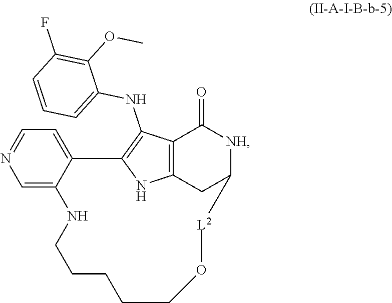

or a pharmaceutically acceptable salt, stereoisomer, solvate, prodrug, isotopic derivative, or tautomer thereof.

In some embodiments, the compound is of Formula (II-A-I-B-b-6):

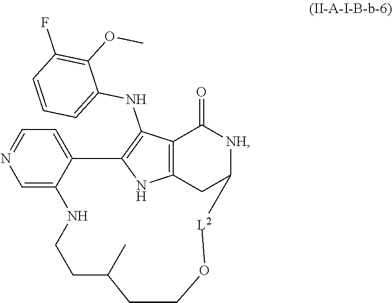

or a pharmaceutically acceptable salt, stereoisomer, solvate, prodrug, isotopic derivative, or tautomer thereof.

In some embodiments, the compound is of Formula (II-A-I-B-b-7):

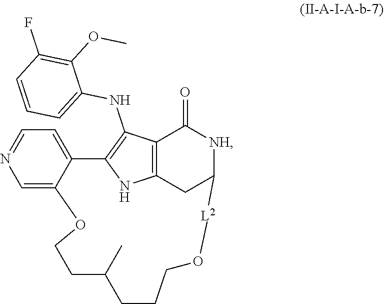

or a pharmaceutically acceptable salt, stereoisomer, solvate, prodrug, isotopic derivative, or tautomer thereof.

In some embodiments, the compound is of Formula (II-A-I-B-b-8):

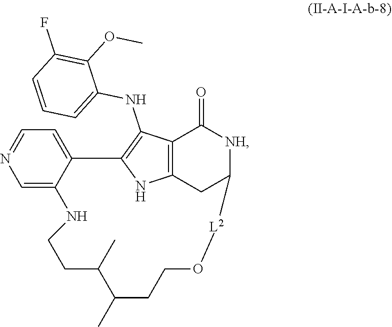

or a pharmaceutically acceptable salt, stereoisomer, solvate, prodrug, isotopic derivative, or tautomer thereof.

In some embodiments, the compound is of Formula (II-A-I-B-b-9):

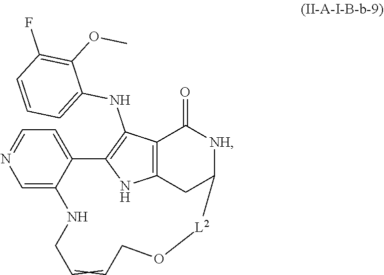

or a pharmaceutically acceptable salt, stereoisomer, solvate, prodrug, isotopic derivative, or tautomer thereof.

In some embodiments, the compound is of Formula (II-A-I-B-b-10):

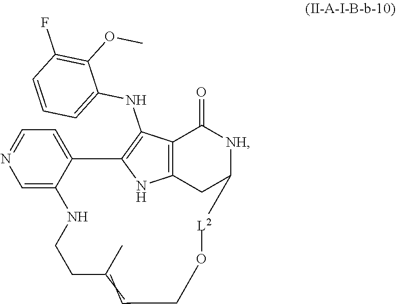

or a pharmaceutically acceptable salt, stereoisomer, solvate, prodrug, isotopic derivative, or tautomer thereof.

In some embodiments, the compound is of Formula (II-A-I-B-b-11):

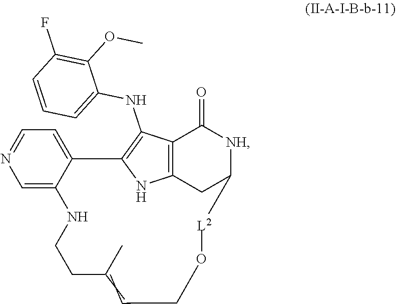

or a pharmaceutically acceptable salt, stereoisomer, solvate, prodrug, isotopic derivative, or tautomer thereof.

In some embodiments, the compound is of Formula (II-A-I-B-b-12):

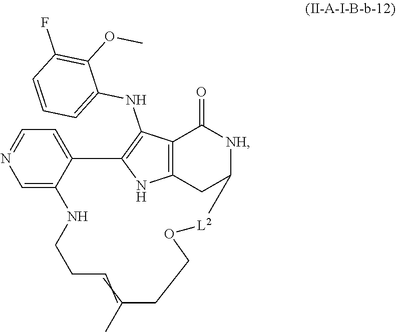

or a pharmaceutically acceptable salt, stereoisomer, solvate, prodrug, isotopic derivative, or tautomer thereof.

In some embodiments, the compound is of Formula (II-A-I-B-b-13):

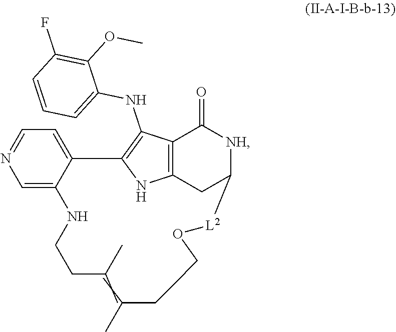

or a pharmaceutically acceptable salt, stereoisomer, solvate, prodrug, isotopic derivative, or tautomer thereof.

In some embodiments, the compound is of Formula (II-A-I-B-b-14):

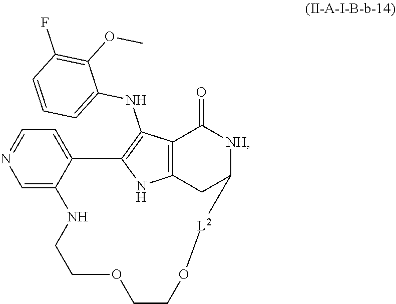

or a pharmaceutically acceptable salt, stereoisomer, solvate, prodrug, isotopic derivative, or tautomer thereof.

In some embodiments, the compound is of Formula (II-A-I-B-b-15):

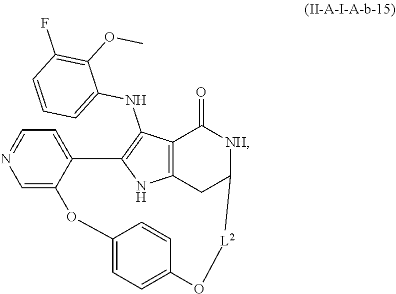

or a pharmaceutically acceptable salt, stereoisomer, solvate, prodrug, isotopic derivative, or tautomer thereof.

In some embodiments, the compound is of Formula (II-A-I-B-b-16):

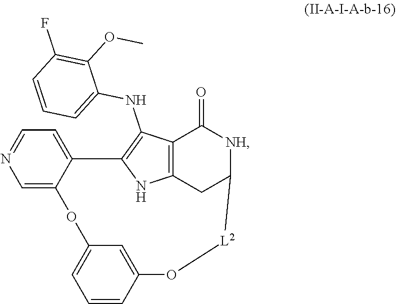

or a pharmaceutically acceptable salt, stereoisomer, solvate, prodrug, isotopic derivative, or tautomer thereof.

In some embodiments, the compound is of Formula (II-A-I-B-b-17):

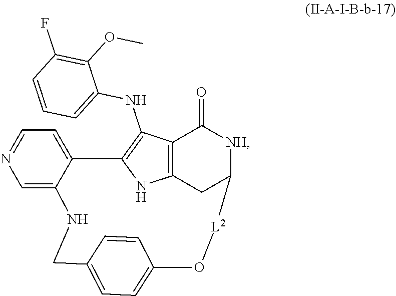

or a pharmaceutically acceptable salt, stereoisomer, solvate, prodrug, isotopic derivative, or tautomer thereof.

In some embodiments, the compound is of Formula (II-A-I-B-b-18):

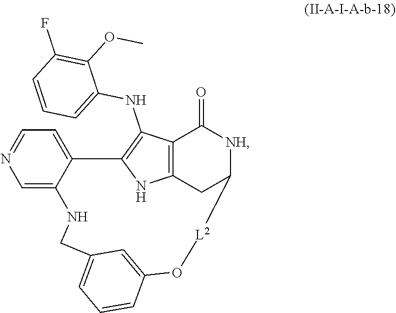

or a pharmaceutically acceptable salt, stereoisomer, solvate, prodrug, isotopic derivative, or tautomer thereof.

In some embodiments, the compound is of Formula (II-A-I-B-b-19):

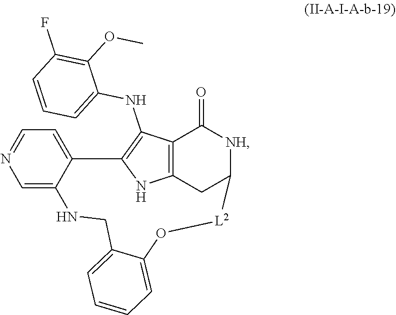

or a pharmaceutically acceptable salt, stereoisomer, solvate, prodrug, isotopic derivative, or tautomer thereof.

In some embodiments, the compound is of Formula (II-A-I-B-c-1):

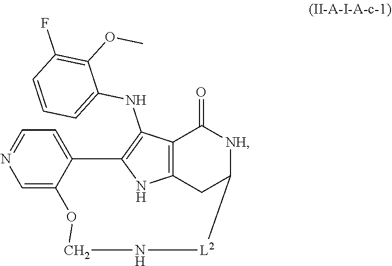

or a pharmaceutically acceptable salt, stereoisomer, solvate, prodrug, isotopic derivative, or tautomer thereof.

In some embodiments, the compound is of Formula (II-A-I-B-c-2):

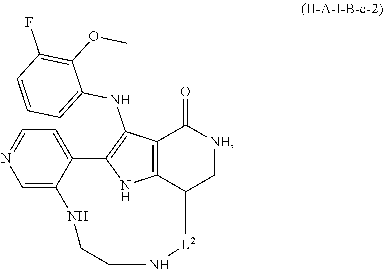

or a pharmaceutically acceptable salt, stereoisomer, solvate, prodrug, isotopic derivative, or tautomer thereof.

In some embodiments, the compound is of Formula (II-A-I-B-c-3):

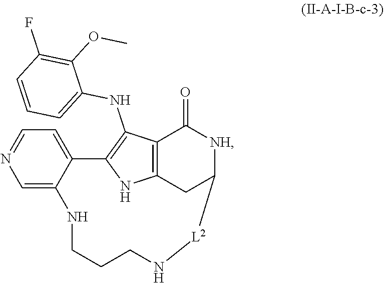

or a pharmaceutically acceptable salt, stereoisomer, solvate, prodrug, isotopic derivative, or tautomer thereof.

In some embodiments, the compound is of Formula (II-A-I-B-c-4):

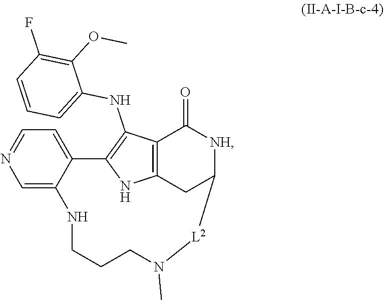

or a pharmaceutically acceptable salt, stereoisomer, solvate, prodrug, isotopic derivative, or tautomer thereof.

In some embodiments, the compound is of Formula (II-A-I-B-c-5):

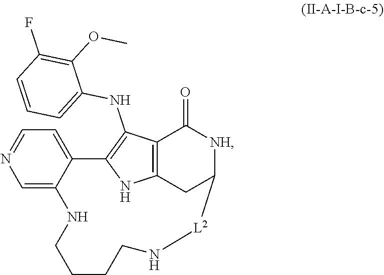

or a pharmaceutically acceptable salt, stereoisomer, solvate, prodrug, isotopic derivative, or tautomer thereof.

In some embodiments, the compound is of Formula (II-A-I-B-c-6):

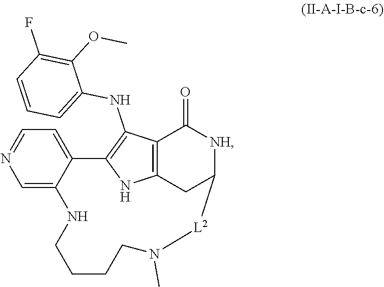

or a pharmaceutically acceptable salt, stereoisomer, solvate, prodrug, isotopic derivative, or tautomer thereof.

In some embodiments, the compound is of Formula (II-A-I-B-c-7):

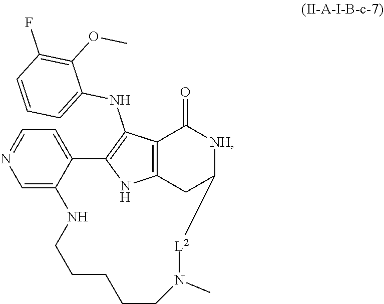

or a pharmaceutically acceptable salt, stereoisomer, solvate, prodrug, isotopic derivative, or tautomer thereof.

In some embodiments, the compound is of Formula (II-A-I-B-d-1):

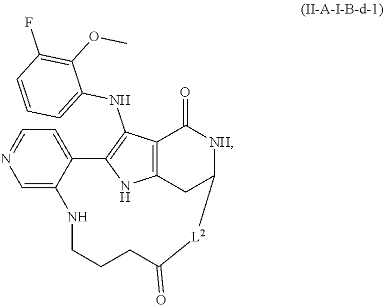

or a pharmaceutically acceptable salt, stereoisomer, solvate, prodrug, isotopic derivative, or tautomer thereof.

In some embodiments, the compound is of Formula (II-A-I-B-d-2):

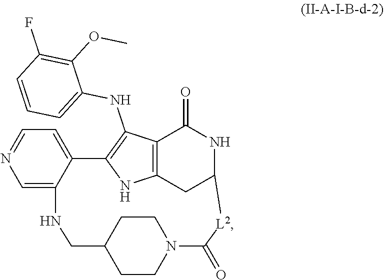

or a pharmaceutically acceptable salt, stereoisomer, solvate, prodrug, isotopic derivative, or tautomer thereof.

In some embodiments, the compound is of Formula (II-A-I-B-d-3):

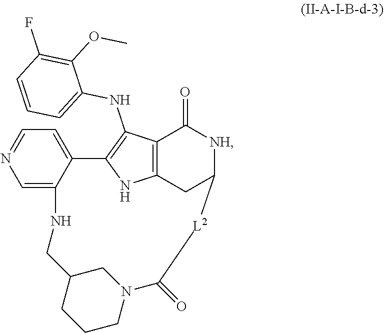

or a pharmaceutically acceptable salt, stereoisomer, solvate, prodrug, isotopic derivative, or tautomer thereof.

In some embodiments, the compound is of Formula (II-A-I-B-e-1):

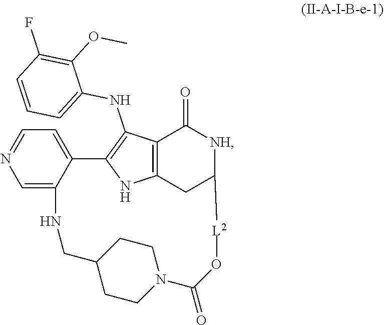

or a pharmaceutically acceptable salt, stereoisomer, solvate, prodrug, isotopic derivative, or tautomer thereof.

In some embodiments, the compound is of Formula (II-A-I-B-e-2):

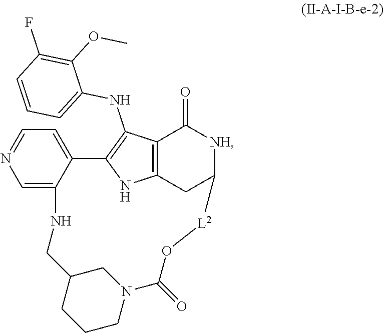

or a pharmaceutically acceptable salt, stereoisomer, solvate, prodrug, isotopic derivative, or tautomer thereof.

In some embodiments, the compound is of Formula (II-A-I-B-f-1):

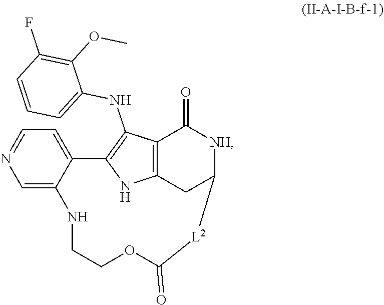

or a pharmaceutically acceptable salt, stereoisomer, solvate, prodrug, isotopic derivative, or tautomer thereof.

In some embodiments, the compound is of Formula (II-A-I-B-g-1):

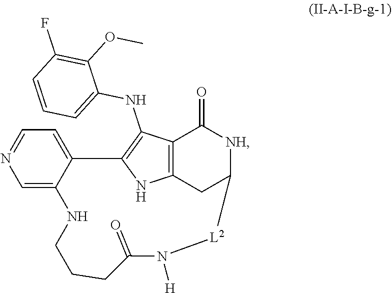

or a pharmaceutically acceptable salt, stereoisomer, solvate, prodrug, isotopic derivative, or tautomer thereof.

In some embodiments, the compound is of Formula (II-A-I-B-g-2):

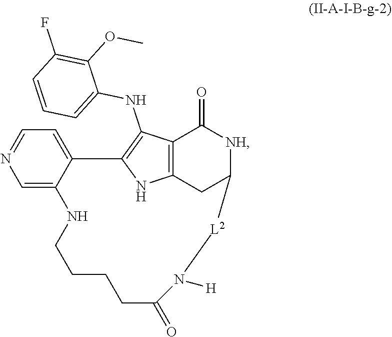

or a pharmaceutically acceptable salt, stereoisomer, solvate, prodrug, isotopic derivative, or tautomer thereof.

In some embodiments, the compound is of Formula (II-A-I-B-h-1):

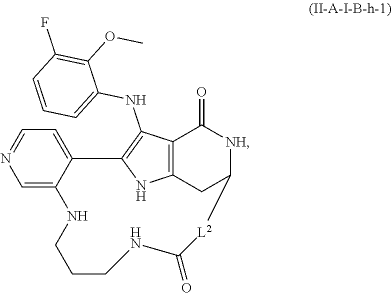

or a pharmaceutically acceptable salt, stereoisomer, solvate, prodrug, isotopic derivative, or tautomer thereof.

In some embodiments, the compound is of Formula (II-A-I-B-h-2):

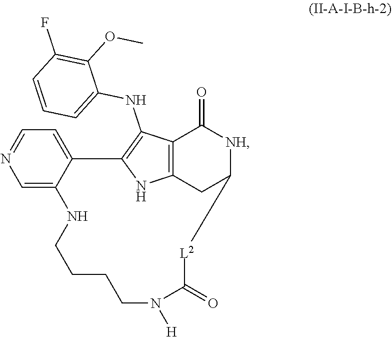

or a pharmaceutically acceptable salt, stereoisomer, solvate, prodrug, isotopic derivative, or tautomer thereof.

In some embodiments, the compound is selected from the compounds described in Table 3 and pharmaceutically acceptable salts, stereoisomers, solvates, prodrugs, isotopic derivatives, or tautomers thereof.

In some embodiments, the compound is selected from the compounds described in Table 3 and prodrugs and pharmaceutically acceptable salts thereof.

In some embodiments, the compound is selected from the compounds described in Table 3 and pharmaceutically acceptable salts thereof.

In some embodiments, the compound is selected from the prodrugs of the compounds described in Table 3 and pharmaceutically acceptable salts thereof.

In some embodiments, the compound is selected from the compounds described in Table 3.

TABLE 3
Certain examples of the compound of Formula A

| # | Structure | IUPAC name |
|---|---|---|
| 1 | 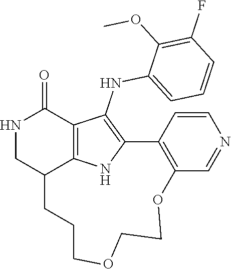 [SMILES: COc1c(F)cccc1Nc1c2[nH]c3c1C(=O)NCC3CCCOCCOc1cnccc1-2] | 22-[(3-fluoro-2-methoxyphenyl)amino]-8,11- dioxa-5,17,21- triazatetracyclo[17.2.1.0^{2,7}.0^{15,20}]docosa- 1(22),2,4,6,19-pentaen-18-one |
| 2 | 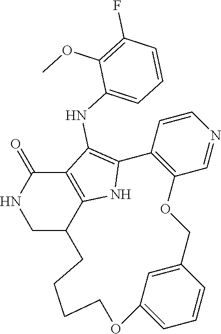 [SMILES: COc1c(F)cccc1Nc1c2[nH]c3c1C(=O)NCC3CCCCOc1cccc(c1)COc1cnccc1-2] | 28-[(3-fluoro-2-methoxyphenyl)amino]-3,22- dioxa-6,11,15- triazapentacyclo[21.3.1.1^{10,13}.0^{4,9}.0^{12,17}]octacosa- 1(27),4,6,8,10(28),12,23,25-octaen-14-one |
| 3 | 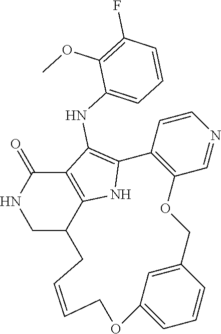 [SMILES: COc1c(F)cccc1Nc1c2[nH]c3c1C(=O)NCC3C/C=C\COc1cccc(c1)COc1cnccc1-2] | (19Z)-28-[(3-fluoro-2- methoxyphenyl)amino]-3,22-dioxa-6,11,15- triazapentacyclo[21.3.1.1^{10,13}.0^{4,9}.0^{12,17}]octacosa- 1(27),4,6,8,10(28),12,19,23,25-nonaen-14- one |
| 4 | 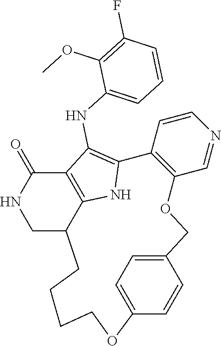 [SMILES: COc1c(F)cccc1Nc1c2[nH]c3c1C(=O)NCC3CCCCOc1ccc(cc1)COc1cnccc1-2] | 28-[(3-fluoro-2-methoxyphenyl)amino]-3,22- dioxa-6,11,15- triazapentacyclo[21.2.2.1^{10,13}.0^{4,9}.0^{12,17}]octaco sa-1(25),4,6,8,10(28),12,23,26-octaen-14-one |
| 5 | 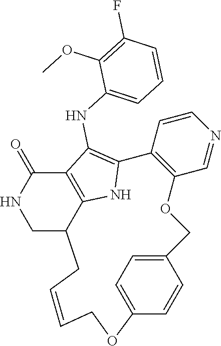 [SMILES: COc1c(F)cccc1Nc1c2[nH]c3c1C(=O)NCC3C/C=C\COc1ccc(cc1)COc1cnccc1-2] | (19Z)-28-[(3-fluoro-2- methoxyphenyl)amino]-3,22-dioxa-6,11,15- triazapentacyclo[21.2.2.1^{10,13}.0^{4,9}.0^{12,17}]octacosa- 1(25),4,6,8,10(28),12,19,23,26-nonaen-14- one |
| 6 | 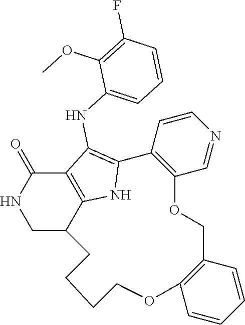 [SMILES: COc1c(F)cccc1Nc1c2[nH]c3c1C(=O)NCC3CCCCOc1ccccc1COc1cnccc1-2] | 28-[(3-fluoro-2-methoxyphenyl)amino]-8,16- dioxa-5,23,27- triazapentacyclo[23.2.1.0^{2,7}.0^{10,15}.0^{21,26}]octacosa- 1(28),2,4,6,10,12,14,25-octaen-24-one |
| 7 | 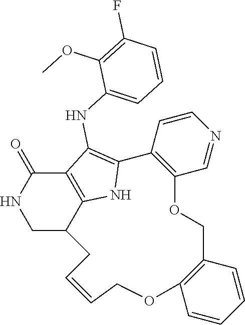 [SMILES: COc1c(F)cccc1Nc1c2[nH]c3c1C(=O)NCC3C/C=C\COc1ccccc1COc1cnccc1-2] | (18Z)-28-[(3-fluoro-2- methoxyphenyl)amino]-8,16-dioxa-5,23,27- triazapentacyclo[23.2.1.0^{2,7}.0^{10,15}.0^{21,26}]octacosa- 1(28),2,4,6,10,12,14,18,25-nonaen-24-one |
| 8 | 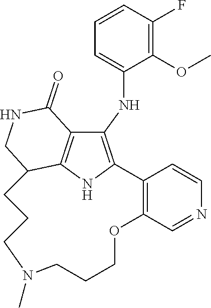 [SMILES: COc1c(F)cccc1Nc1c2[nH]c3c1C(=O)NCC3CCCN(C)CCCOc1cnccc1-2] | 23-[(3-fluoro-2-methoxyphenyl)amino]-12- methyl-8-oxa-5,12,18,22- tetraazatetracyclo[18.2.1.0^{2,7}.0^{16,21}]tricosa- 1(23),2,4,6,20-pentaen-19-one |
| 9 | 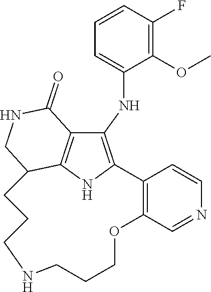 [SMILES: COc1c(F)cccc1Nc1c2[nH]c3c1C(=O)NCC3CCCNCCCOc1cnccc1-2] | 23-[(3-fluoro-2-methoxyphenyl)amino]-8- oxa-5,12,18,22- tetraazatetracyclo[18.2.1.0^{2,7}.0^{16,21}]tricosa- 1(23),2,4,6,20-pentaen-19-one |
| 10 | 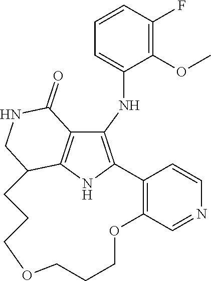 [SMILES: COc1c(F)cccc1Nc1c2[nH]c3c1C(=O)NCC3CCCOCCCOc1cnccc1-2] | 23-[(3-fluoro-2-methoxyphenyl)amino]-8,12- dioxa-5,18,22- triazatetracyclo[18.2.1.0^{2,7}.0^{16,21}]tricosa- 1(23),2,4,6,20-pentaen-19-one |
| 11 | 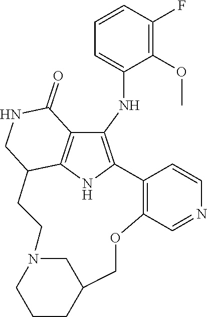 [SMILES: COc1c(F)cccc1Nc1c2[nH]c3c1C(=O)NCC3CCN1CCCC(COc3cnccc3-2)C1] | 25-[(3-fluoro-2-methoxyphenyl)amino]-18- oxa-1,6,10,15- tetraazapentacyclo[18.3.1.1^{8,11}.0^{4,9}.0^{12,17}]- pentacosa-8,11(25),12,14,16-pentaen-7-one |
| 12 | 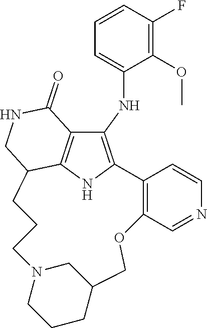 [SMILES: COc1c(F)cccc1Nc1c2[nH]c3c1C(=O)NCC3CCCN1CCCC(COc3cnccc3-2)C1] | 26-[(3-fluoro-2-methoxyphenyl)amino]-3- oxa-6,11,15,21- tetraazapentacyclo[19.3.1.1^{10,13}.0^{4,9}.0^{12,17}]- hexacosa-4,6,8,10(26),12-pentaen-14-one |
| 13 | 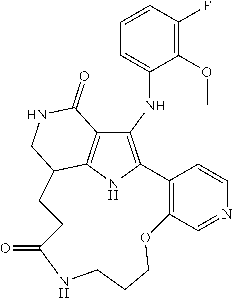 [SMILES: COc1c(F)cccc1Nc1c2[nH]c3c1C(=O)NCC3CCC(=O)NCCCOc1cnccc1-2] | 23-[(3-fluoro-2-methoxyphenyl)amino]-8- oxa-5,12,18,22- tetraazatetracyclo[18.2.1.0^{2,7}.0^{16,21}]tricosa- 1(23),2,4,6,20-pentaene-13,19-dione |
| 14 | 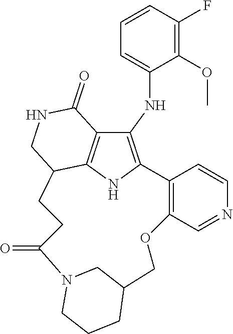 [SMILES: COc1c(F)cccc1Nc1c2[nH]c3c1C(=O)NCC3CCC(=O)N1CCCC(COc3cnccc3-2)C1] | 26-[(3-fluoro-2-methoxyphenyl)amino]-3- oxa-6,11,15,21- tetraazapentacyclo[19.3.1.1^{10,13}.0^{4,9}.0^{12,17}]- hexacosa-4,6,8,10(26),12-pentaene-14,20- dione |
| 15 | 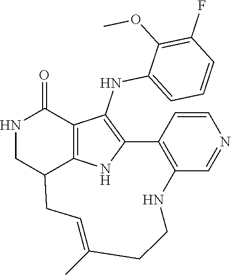 [SMILES: COc1c(F)cccc1Nc1c2[nH]c3c1C(=O)NCC3C/C=C(\C)CCNc1cnccc1-2] | (11E)-21-[(3-fluoro-2- methoxyphenyl)amino]-11-methyl-5,8,16,20- tetraazatetracyclo[16.2.1.0^{2,7}.0^{14,19}]henicosa- 1(21),2,4,6,11,18-hexaen-17-one |
| 16 | 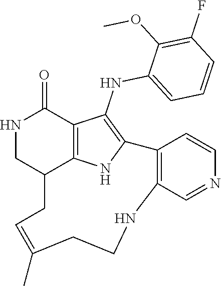 [SMILES: COc1c(F)cccc1Nc1c2[nH]c3c1C(=O)NCC3C/C=C(/C)CCNc1cnccc1-2] | (11Z)-21-[(3-fluoro-2- methoxyphenyl)amino]-11-methyl-5,8,16,20- tetraazatetracyclo[16.2.1.0^{2,7}.0^{14,19}]henicosa- 1(21),2,4,6,11,18-hexaen-17-one |
| 17 | 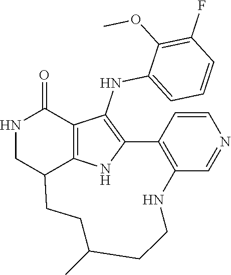 [SMILES: COc1c(F)cccc1Nc1c2[nH]c3c1C(=O)NCC3CCC(C)CCNc1cnccc1-2] | 21-[(3-fluoro-2-methoxyphenyl)amino]-11- methyl-5,8,16,20- tetraazatetracyclo[16.2.1.0^{2,7}.0^{14,19}]henicosa- 1(21),2,4,6,18-pentaen-17-one |
| 18 | 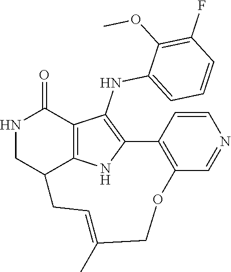 [SMILES: COc1c(F)cccc1Nc1c2[nH]c3c1C(=O)NCC3C/C=C(\C)COc1cnccc1-2] | 20-[(3-fluoro-2-methoxyphenyl)amino]-10- methyl-8-oxa-5,15,19- triazatetracyclo[15.2.1.0^{2,7}.0^{13,18}]icosa- 1(20),2,4,6,10,17-hexaen-16-one |
| 19 | 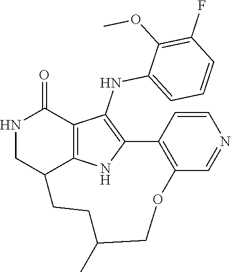 [SMILES: COc1c(F)cccc1Nc1c2[nH]c3c1C(=O)NCC3CCC(C)COc1cnccc1-2] | 20-[(3-fluoro-2-methoxyphenyl)amino]-10- methyl-8-oxa-5,15,19- triazatetracyclo[15.2.1.0^{2,7}.0^{13,18}]icosa- 1(20),2,4,6,17-pentaen-16-one |
| 20 | 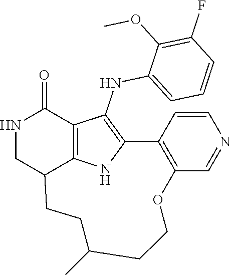 [SMILES: COc1c(F)cccc1Nc1c2[nH]c3c1C(=O)NCC3CCC(C)CCOc1cnccc1-2] | 21-[(3-fluoro-2-methoxyphenyl)amino]-11- methyl-8-oxa-5,16,20- triazatetracyclo[16.2.1.0^{2,7}.0^{14,19}]henicosa- 1(21),2,4,6,18-pentaen-17-one |
| 21 | 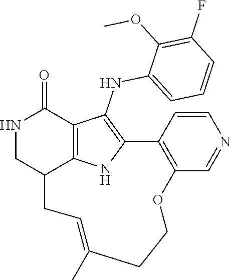 [SMILES: COc1c(F)cccc1Nc1c2[nH]c3c1C(=O)NCC3C/C=C(\C)CCOc1cnccc1-2] | (11E)-21-[(3-fluoro-2- methoxyphenyl)amino]-11-methyl-8-oxa- 5,16,20- triazatetracyclo[16.2.1.0^{2,7}.0^{14,19}]henicosa- 1(21),2,4,6,11,18-hexaen-17-one |
| 22 | 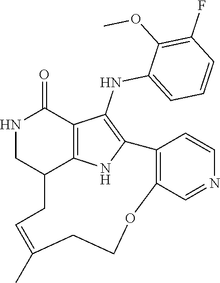 [SMILES: COc1c(F)cccc1Nc1c2[nH]c3c1C(=O)NCC3C/C=C(/C)CCOc1cnccc1-2] | (11Z)-21-[(3-fluoro-2- methoxyphenyl)amino]-11-methyl-8-oxa- 5,16,20- triazatetracyclo[16.2.1.0^{2,7}.0^{14,19}]henicosa- 1(21),2,4,6,11,18-hexaen-17-one |
| 23 | 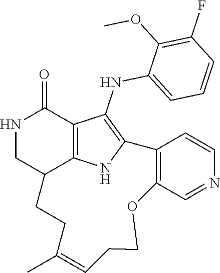 [SMILES: COc1c(F)cccc1Nc1c2[nH]c3c1C(=O)NCC3CC/C(C)=C\CCOc1cnccc1-2] | (11Z)-22-[(3-fluoro-2- methoxyphenyl)amino]-12-methyl-8-oxa- 5,17,21- triazatetracyclo[17.2.1.0^{2,7}.0^{15,20}]docosa- 1(22),2,4,6,11,19-hexaen-18-one |
| 24 | 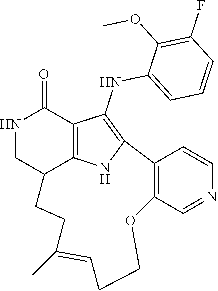 [SMILES: COc1c(F)cccc1Nc1c2[nH]c3c1C(=O)NCC3CC/C(C)=C/CCOc1cnccc1-2] | (11E)-22-[(3-fluoro-2- methoxyphenyl)amino]-12-methyl-8-oxa- 5,17,21- triazatetracyclo[17.2.1.0^{2,7}.0^{15,20}]docosa- 1(22),2,4,6,11,19-hexaen-18-one |
| 25 | 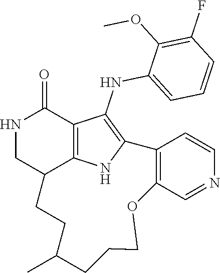 [SMILES: COc1c(F)cccc1Nc1c2[nH]c3c1C(=O)NCC3CCC(C)CCCOc1cnccc1-2] | 22-[(3-fluoro-2-methoxyphenyl)amino]-12- methyl-8-oxa-5,17,21- triazatetracyclo[17.2.1.0^{2,7}.0^{15,20}]docosa- 1(22),2,4,6,19-pentaen-18-one |
| 26 | 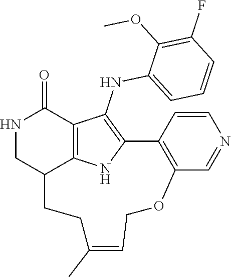 [SMILES: COc1c(F)cccc1Nc1c2[nH]c3c1C(=O)NCC3CC/C(C)=C\COc1cnccc1-2] | (10Z)-21-[(3-fluoro-2- methoxyphenyl)amino]-11-methyl-8-oxa- 5,16,20- triazatetracyclo[16.2.1.0^{2,7}.0^{14,19}]henicosa- 1(21),2,4,6,10,18-hexaen-17-one |
| 27 | 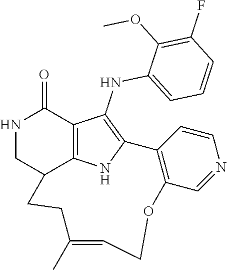 [SMILES: COc1c(F)cccc1Nc1c2[nH]c3c1C(=O)NCC3CC/C(C)=C/COc1cnccc1-2] | (10E)-21-[(3-fluoro-2- methoxyphenyl)amino]-11-methyl-8-oxa- 5,16,20- triazatetracyclo[16.2.1.0^{2,7}.0^{14,19}]henicosa- 1(21),2,4,6,10,18-hexaen-17-one |
| 28 | 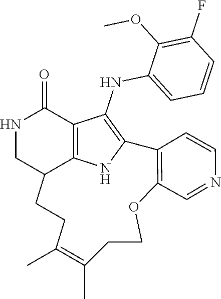 [SMILES: COc1c(F)cccc1Nc1c2[nH]c3c1C(=O)NCC3CC/C(C)=C(/C)CCOc1cnccc1-2] | (11Z)-22-[(3-fluoro-2- methoxyphenyl)amino]-11,12-dimethyl-8- oxa-5,17,21- triazatetracyclo[17.2.1.0^{2,7}.0^{15,20}]docosa- 1(22),2,4,6,11,19-hexaen-18-one |
| 29 | 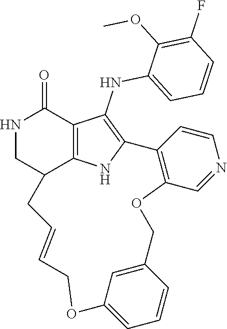 [SMILES: COc1c(F)cccc1Nc1c2[nH]c3c1C(=O)NCC3C/C=C/COc1cccc(c1)COc1cnccc1-2] | (19E)-28-[(3-fluoro-2- methoxyphenyl)amino]-3,22-dioxa-6,11,15- triazapentacyclo[21.3.1.1^{10,13}.0^{4,9}.0^{12,17}]octacosa- 1(27),4,6,8,10(28),12,19,23,25-nonaen-14- one |
| 30 | 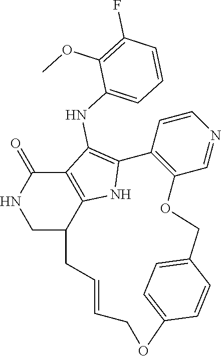 [SMILES: COc1c(F)cccc1Nc1c2[nH]c3c1C(=O)NCC3C/C=C/COc1ccc(cc1)COc1cnccc1-2] | (19E)-28-[(3-fluoro-2- methoxyphenyl)amino]-3,22-dioxa-6,11,15- triazapentacyclo[21.2.2.1^{10,13}.0^{4,9}.0^{12,17}]octacosa- 1(25),4,6,8,10(28),12,19,23,26-nonaen-14- one |
| 31 | 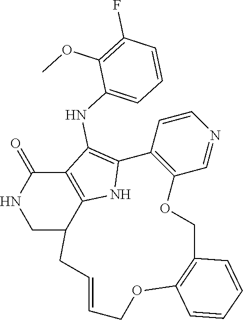 [SMILES: COc1c(F)cccc1Nc1c2[nH]c3c1C(=O)NCC3C/C=C/COc1ccccc1COc1cnccc1-2] | (18E)-28-[(3-fluoro-2- methoxyphenyl)amino]-8,16-dioxa-5,23,27- triazapentacyclo[23.2.1.0^{2,7}.^{10,15}.0_{21,26}]octacosa- 1(28),2,4,6,10,12,14,18,25-nonaen-24-one |
| 32 | 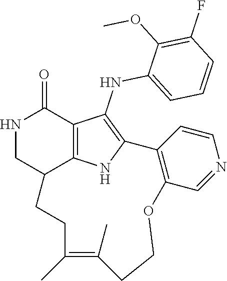 [SMILES: COc1c(F)cccc1Nc1c2[nH]c3c1C(=O)NCC3CC/C(C)=C(\C)CCOc1cnccc1-2] | (11E)-22-[(3-fluoro-2- methoxyphenyl)amino]-11,12-dimethyl-8- oxa-5,17,21- triazatetracyclo[17.2.1.0^{2,7}.0^{15,20}]docosa- 1(22),2,4,6,11,19-hexaen-18-one |
| 33 | 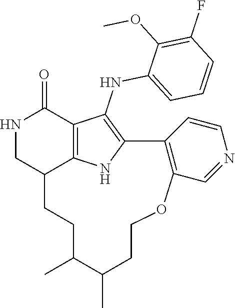 [SMILES: COc1c(F)cccc1Nc1c2[nH]c3c1C(=O)NCC3CCC(C)C(C)CCOc1cnccc1-2] | 22-[(3-fluoro-2-methoxyphenyl)amino]- 11,12-dimethyl-8-oxa-5,17,21- triazatetracyclo[17.2.1.0^{2,7}.0^{15,20}]docosa- 1(22),2,4,6,19-pentaen-18-one |
| 34 | 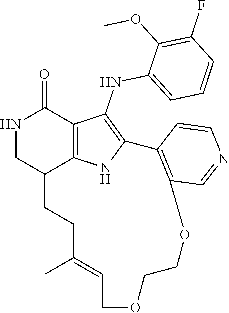 [SMILES: COc1c(F)cccc1Nc1c2[nH]c3c1C(=O)NCC3CC/C(C)=C/COCCOc1cnccc1-2] | (13E)-24-[(3-fluoro-2- methoxyphenyl)amino]-14-methyl-8,11- dioxa-5,19,23- triazatetracyclo[19.2.1.0^{2,7}.0^{17,22}]tetracosa- 1(24),2,4,6,13,21-hexaen-20-one |
| 35 | 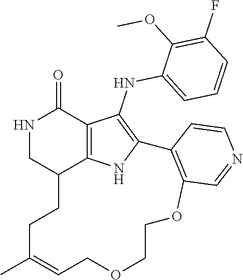 [SMILES: COc1c(F)cccc1Nc1c2[nH]c3c1C(=O)NCC3CC/C(C)=C\COCCOc1cnccc1-2] | (13Z)-24-[(3-fluoro-2- methoxyphenyl)amino]-14-methyl-8,11- dioxa-5,19,23- triazatetracyclo[19.2.1.0^{2,7}.0^{17,22}]tetracosa- 1(24),2,4,6,13,21-hexaen-20-one |
| 36 | 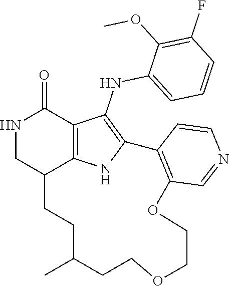 [SMILES: COc1c(F)cccc1Nc1c2[nH]c3c1C(=O)NCC3CCC(C)CCOCCOc1cnccc1-2] | 24-[(3-fluoro-2-methoxyphenyl)amino]-14- methyl-8,11-dioxa-5,19,23- triazatetracyclo[19.2.1.0^{2,7}.0^{17,22}]tetracosa- 1(24),2,4,6,21-pentaen-20-one |
| 37 | 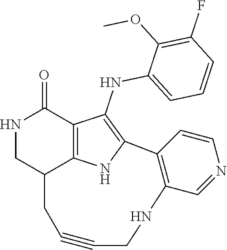 [SMILES: COc1c(F)cccc1Nc1c2[nH]c3c1C(=O)NCC3CC#CCNc1cnccc1-2] | 20-[(3-fluoro-2-methoxyphenyl)amino]- 5,8,15,19- tetraazatetracyclo[15.2.1.0^{2,7}.0^{13,18}]icosa- 1(20),2,4,6,17-pentaen-10-yn-16-one |
| 38 | 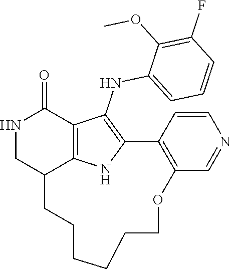 [SMILES: COc1c(F)cccc1Nc1c2[nH]c3c1C(=O)NCC3CCCCCCOc1cnccc1-2] | 22-[(3-fluoro-2-methoxyphenyl)amino]-8- oxa-5,17,21- triazatetracyclo[17.2.1.0^{2,7}.0^{15,20}]docosa- 1(22),2,4,6,19-pentaen-18-one |
| 39 | 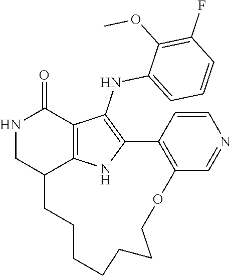 [SMILES: COc1c(F)cccc1Nc1c2[nH]c3c1C(=O)NCC3CCCCCCCOc1cnccc1-2] | 23-[(3-fluoro-2-methoxyphenyl)amino]-8- oxa-5,18,22- triazatetracyclo[18.2.1.0^{2,7}.0^{16,21}]tricosa- 1(23),2,4,6,20-pentaen-19-one |
| 40 | 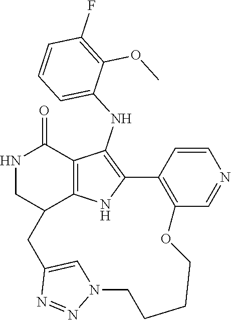 [SMILES: COc1c(F)cccc1Nc1c2[nH]c3c1C(=O)NCC3Cc1cn(nn1)CCCCOc1cnccc1-2] | 25-[(3-fluoro-2-methoxyphenyl)amino]-8- oxa-5,13,14,15,20,24- hexaazapentacyclo[20.2.1.1^{13,16}.0^{2,7}.0^{18,23}]- hexacosa-1(25),2,4,6,14,16(26),22-heptaen- 21-one |
| 41 | 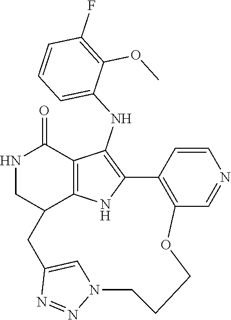 [SMILES: COc1c(F)cccc1Nc1c2[nH]c3c1C(=O)NCC3Cc1cn(nn1)CCCOc1cnccc1-2] | 24-[(3-fluoro-2-methoxyphenyl)amino]-8- oxa-5,12,13,14,19,23- hexaazapentacyclo[19.2.1.1^{12,15}.0^{2,7}.0^{17,22}]- pentacosa-1(24),2,4,6,13,15(25),21-heptaen- 20-one |
| 42 | 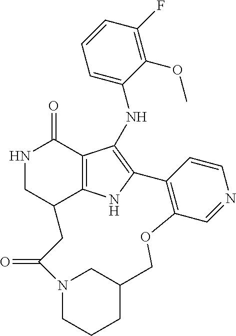 [SMILES: COc1c(F)cccc1Nc1c2[nH]c3c1C(=O)NCC3CC(=O)N1CCCC(COc3cnccc3-2)C1] | 25-[(3-fluoro-2-methoxyphenyl)amino]-18- oxa-1,6,10,15- tetraazapentacyclo[18.3.1.1^{8,11}.0^{4,9}.0^{12,17}]- pentacosa-8,11(25),12,14,16-pentaene-2,7- dione |
| 43 | 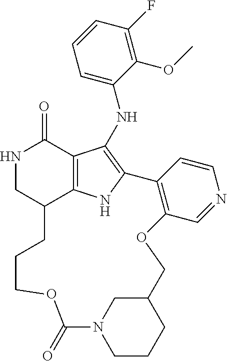 [SMILES: COc1c(F)cccc1Nc1c2[nH]c3c1C(=O)NCC3CCCOC(=O)N1CCCC(COc3cnccc3-2)C1] | 28-[(3-fluoro-2-methoxyphenyl)amino]-3,21- dioxa-6,11,15,23- tetraazapentacyclo[21.3.1.1^{10,13}.0^{4,9}.0^{12,17}]- octacosa-4,6,8,10(28),12-pentaene-14,22- dione |
| 44 | 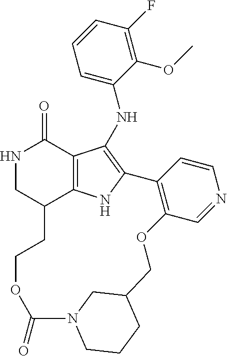 [SMILES: COc1c(F)cccc1Nc1c2[nH]c3c1C(=O)NCC3CCOC(=O)N1CCCC(COc3cnccc3-2)C1] | 27-[(3-fluoro-2-methoxyphenyl)amino]-3,20- dioxa-6,11,15,22- tetraazapentacyclo[20.3.1.1^{10,13}.0^{4,9}.0^{12,17}]- heptacosa-4,6,8,10(27),12-pentaene-14,21- dione |
| 45 | 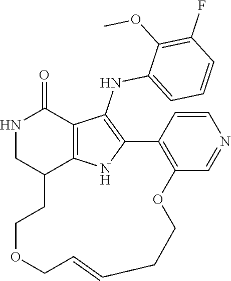 [SMILES: COc1c(F)cccc1Nc1c2[nH]c3c1C(=O)NCC3CCOC/C=C/CCOc1cnccc1-2] | (11E)-24-[(3-fluoro-2- methoxyphenyl)amino]-8,14-dioxa-5,19,23- triazatetracyclo[19.2.1.0^{2,7}.0^{17,22}]tetracosa- 1(24),2,4,6,11,21-hexaen-20-one |
| 46 | 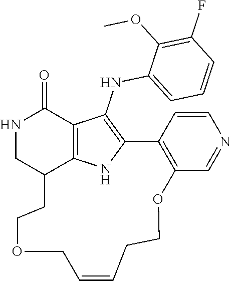 [SMILES: COc1c(F)cccc1Nc1c2[nH]c3c1C(=O)NCC3CCOC/C=C\CCOc1cnccc1-2] | (11Z)-24-[(3-fluoro-2- methoxyphenyl)amino]-8,14-dioxa-5,19,23- triazatetracyclo[19.2.1.0^{2,7}.0^{17,22}]tetracosa- 1(24),2,4,6,11,21-hexaen-20-one |
| 47 | 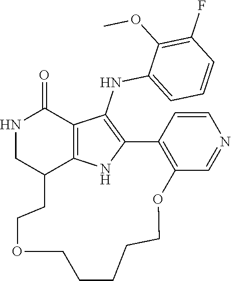 [SMILES: COc1c(F)cccc1Nc1c2[nH]c3c1C(=O)NCC3CCOCCCCCOc1cnccc1-2] | 24-[(3-fluoro-2-methoxyphenyl)amino]-8,14- dioxa-5,19,23- triazatetracyclo[19.2.1.0^{2,7}.0^{17,22}]tetracosa- 1(24),2,4,6,21-pentaen-20-one |
| 48 | 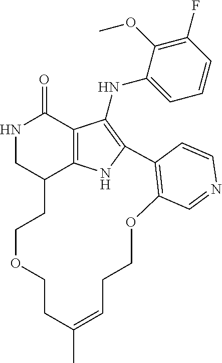 [SMILES: COc1c(F)cccc1Nc1c2[nH]c3c1C(=O)NCC3CCOCC/C(C)=C\CCOc1cnccc1-2] | (11Z)-25-[(3-fluoro-2- methoxyphenyl)amino]-12-methyl-8,15- dioxa-5,20,24- triazatetracyclo[20.2.1.0^{2,7}.0^{18,23}]pentacosa- 1(25),2,4,6,11,22-hexaen-21-one |
| 49 | 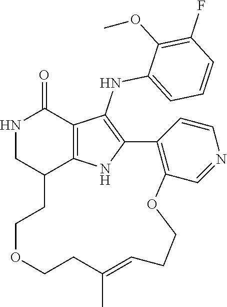 [SMILES: COc1c(F)cccc1Nc1c2[nH]c3c1C(=O)NCC3CCOCC/C(C)=C/CCOc1cnccc1-2] | (11E)-25-[(3-fluoro-2- methoxyphenyl)amino]-12-methyl-8,15- dioxa-5,20,24- triazatetracyclo[20.2.1.0^{2,7}.0^{18,23}]pentacosa- 1(25),2,4,6,11,22-hexaen-21-one |
| 50 | 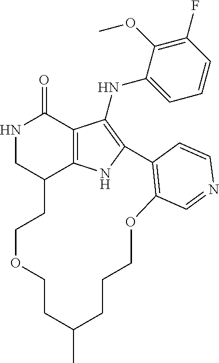 [SMILES: COc1c(F)cccc1Nc1c2[nH]c3c1C(=O)NCC3CCOCCC(C)CCCOc1cnccc1-2] | 25-[(3-fluoro-2-methoxyphenyl)amino]-12- methyl-8,15-dioxa-5,20,24- triazatetracyclo[20.2.1.0^{2,7}.0^{18,23}]pentacosa- 1(25),2,4,6,22-pentaen-21-one |
| 51 | 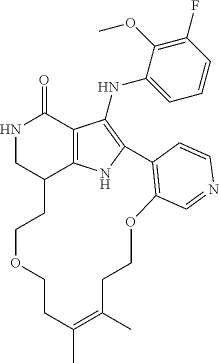 [SMILES: COc1c(F)cccc1Nc1c2[nH]c3c1C(=O)NCC3CCOCC/C(C)=C(/C)CCOc1cnccc1-2] | (11Z)-25-[(3-fluoro-2- methoxyphenyl)amino]-11,12-dimethyl-8,15- dioxa-5,20,24- triazatetracyclo[20.2.1.0^{2,7}.0^{18,23}]pentacosa- 1(25),2,4,6,11,22-hexaen-21-one |
| 52 | 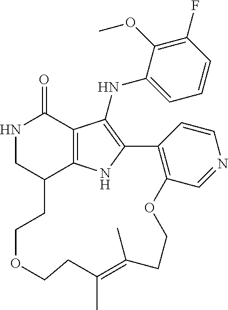 [SMILES: COc1c(F)cccc1Nc1c2[nH]c3c1C(=O)NCC3CCOCC/C(C)=C(\C)CCOc1cnccc1-2] | (11E)-25-[(3-fluoro-2- methoxyphenyl)amino]-11,12-dimethyl-8,15- dioxa-5,20,24- triazatetracyclo[20.2.1.0^{2,7}.0^{18,23}]pentacosa- 1(25),2,4,6,11,22-hexaen-21-one |
| 53 | 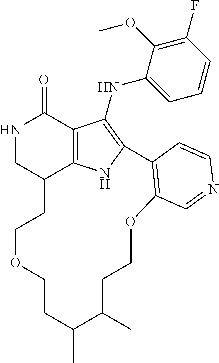 [SMILES: COc1c(F)cccc1Nc1c2[nH]c3c1C(=O)NCC3CCOCCC(C)C(C)CCOc1cnccc1-2] | 25-[(3-fluoro-2-methoxyphenyl)amino]- 11,12-dimethyl-8,15-dioxa-5,20,24- triazatetracyclo[20.2.1.0^{2,7}.0^{18,23}]pentacosa- 1(25),2,4,6,22-pentaen-21-one |
| 54 | 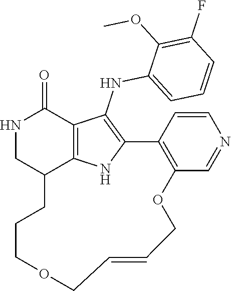 [SMILES: COc1c(F)cccc1Nc1c2[nH]c3c1C(=O)NCC3CCCOC/C=C/COc1cnccc1-2] | (10E)-24-[(3-fluoro-2- methoxyphenyl)amino]-8,13-dioxa-5,19,23- triazatetracyclo[19.2.1.0^{2,7}.0^{17,22}]tetracosa- 1(24),2,4,6,10,21-hexaen-20-one |
| 55 | 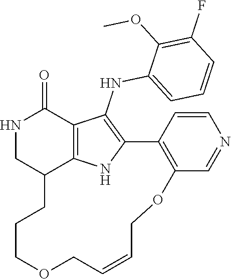 [SMILES: COc1c(F)cccc1Nc1c2[nH]c3c1C(=O)NCC3CCCOC/C=C\COc1cnccc1-2] | (10Z)-24-[(3-fluoro-2- methoxyphenyl)amino]-8,13-dioxa-5,19,23- triazatetracyclo[19.2.1.0^{2,7}.0^{17,22}]tetracosa- 1(24),2,4,6,10,21-hexaen-20-one |
| 56 | 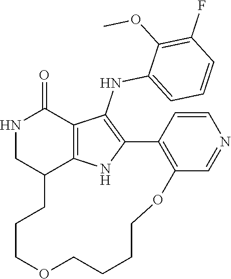 [SMILES: COc1c(F)cccc1Nc1c2[nH]c3c1C(=O)NCC3CCCOCCCCOc1cnccc1-2] | 24-[(3-fluoro-2-methoxyphenyl)amino]-8,13- dioxa-5,19,23- triazatetracyclo[19.2.1.0^{2,7}.0^{17,22}]tetracosa- 1(24),2,4,6,21-pentaen-20-one |
| 57 | 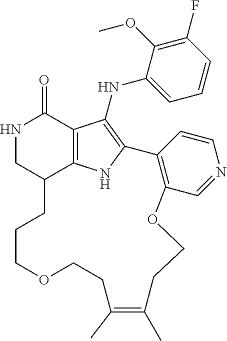 [SMILES: COc1c(F)cccc1Nc1c2[nH]c3c1C(=O)NCC3CCCOCC/C(C)=C(/C)CCOc1cnccc1-2] | (11Z)-26-[(3-fluoro-2- methoxyphenyl)amino]-11,12-dimethyl-8,15- dioxa-5,21,25- triazatetracyclo[21.2.1.0^{2,7}.0^{19,24}]hexacosa- 1(26),2,4,6,11,23-hexaen-22-one |
| 58 | 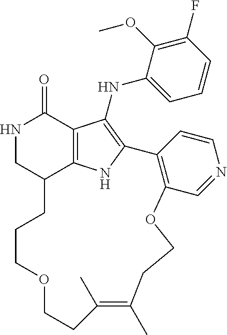 [SMILES: COc1c(F)cccc1Nc1c2[nH]c3c1C(=O)NCC3CCCOCC/C(C)=C(\C)CCOc1cnccc1-2] | (11E)-26-[(3-fluoro-2- methoxyphenyl)amino]-11,12-dimethyl-8,15- dioxa-5,21,25- triazatetracyclo[21.2.1.0^{2,7}.0^{19,24}]hexacosa- 1(26),2,4,6,11,23-hexaen-22-one |
| 59 | 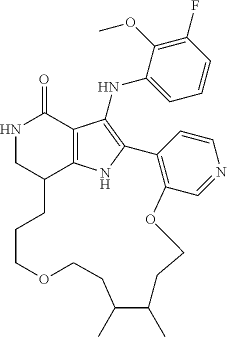 [SMILES: COc1c(F)cccc1Nc1c2[nH]c3c1C(=O)NCC3CCCOCCC(C)C(C)CCOc1cnccc1-2] | 26-[(3-fluoro-2-methoxyphenyl)amino]- 11,12-dimethyl-8,15-dioxa-5,21,25- triazatetracyclo[21.2.1.0^{2,7}.0^{19,24}]hexacosa- 1(26),2,4,6,23-pentaen-22-one |
| 60 | 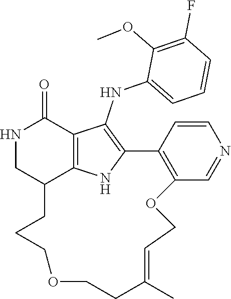 [SMILES: COc1c(F)cccc1Nc1c2[nH]c3c1C(=O)NCC3CCCOCC/C(C)=C/COc1cnccc1-2] | (10E)-25-[(3-fluoro-2- methoxyphenyl)amino]-11-methyl-8,14- dioxa-5,20,24- triazatetracyclo[20.2.1.0^{2,7}.0^{18,23}]pentacosa- 1(25),2,4,6,10,22-hexaen-21-one |
| 61 | 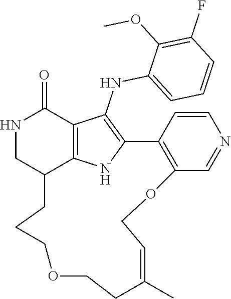 [SMILES: COc1c(F)cccc1Nc1c2[nH]c3c1C(=O)NCC3CCCOCC/C(C)=C\COc1cnccc1-2] | (10Z)-25-[(3-fluoro-2- methoxyphenyl)amino]-11-methyl-8,14- dioxa-5,20,24- triazatetracyclo[20.2.1.0^{2,7}.0^{18,23}]pentacosa- 1(25),2,4,6,10,22-hexaen-21-one |
| 62 | 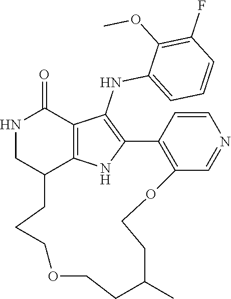 [SMILES: COc1c(F)cccc1Nc1c2[nH]c3c1C(=O)NCC3CCCOCCC(C)CCOc1cnccc1-2] | 25-[(3-fluoro-2-methoxyphenyl)amino]-11- methyl-8,14-dioxa-5,20,24- triazatetracyclo[20.2.1.0^{2,7}.0^{18,23}]pentacosa- 1(25),2,4,6,22-pentaen-21-one |
| 63 | 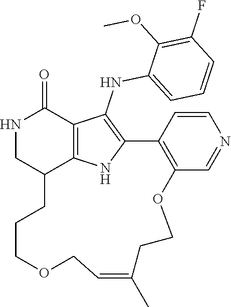 [SMILES: COc1c(F)cccc1Nc1c2[nH]c3c1C(=O)NCC3CCCOC/C=C(/C)CCOc1cnccc1-2] | (11Z)-25-[(3-fluoro-2- methoxyphenyl)amino]-11-methyl-8,14- dioxa-5,20,24- triazatetracyclo[20.2.1.0^{2,7}.0^{18,23}]pentacosa- 1(25),2,4,6,11,22-hexaen-21-one |
| 64 | 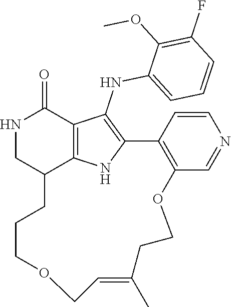 [SMILES: COc1c(F)cccc1Nc1c2[nH]c3c1C(=O)NCC3CCCOC/C=C(\C)CCOc1cnccc1-2] | (11E)-25-[(3-fluoro-2- methoxyphenyl)amino]-11-methyl-8,14- dioxa-5,20,24- triazatetracyclo[20.2.1.0^{2,7}.0^{18,23}]pentacosa- 1(25),2,4,6,11,22-hexaen-21-one |
| 65 | 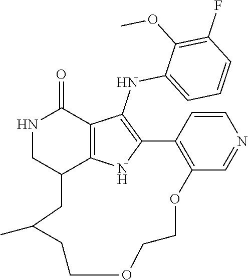 [SMILES: COc1c(F)cccc1Nc1c2[nH]c3c1C(=O)NCC3CC(C)CCOCCOc1cnccc1-2] | 23-[(3-fluoro-2-methoxyphenyl)amino]-14- methyl-8,11-dioxa-5,18,22- triazatetracyclo[18.2.1.0^{2,7}.0^{16,21}]tricosa- 1(23),2,4,6,20-pentaen-19-one |
| 66 | 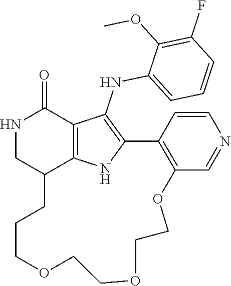 [SMILES: COc1c(F)cccc1Nc1c2[nH]c3c1C(=O)NCC3CCCOCCOCCOc1cnccc1-2] | 25-[(3-fluoro-2-methoxyphenyl)amino]- 8,11,14-trioxa-5,20,24- triazatetracyclo[20.2.1.0^{2,7}.0^{18,23}]pentacosa- 1(25),2,4,6,22-pentaen-21-one |
| 67 | 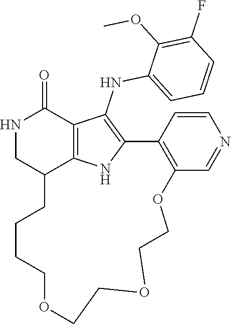 [SMILES: COc1c(F)cccc1Nc1c2[nH]c3c1C(=O)NCC3CCCCOCCOCCOc1cnccc1-2] | 26-[(3-fluoro-2-methoxyphenyl)amino]- 8,11,14-trioxa-5,21,25- triazatetracyclo[21.2.1.0^{2,7}.0^{19,24}]hexacosa- 1(26),2,4,6,23-pentaen-22-one |
| 68 | 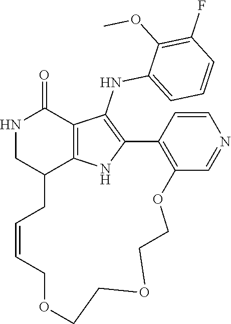 [SMILES: COc1c(F)cccc1Nc1c2[nH]c3c1C(=O)NCC3C/C=C\COCCOCCOc1cnccc1-2] | (16Z)-26-[(3-fluoro-2- methoxyphenyl)amino]-8,11,14-trioxa- 5,21,25- triazatetracyclo[21.2.1.0^{2,7}.0^{19,24}]hexacosa- 1(26),2,4,6,16,23-hexaen-22-one |
| 69 | 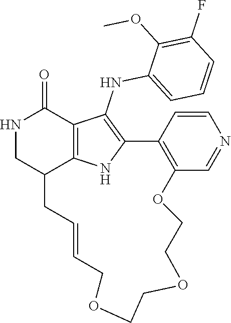 [SMILES: COc1c(F)cccc1Nc1c2[nH]c3c1C(=O)NCC3C/C=C/COCCOCCOc1cnccc1-2] | (16E)-26-[(3-fluoro-2- methoxyphenyl)amino]-8,11,14-trioxa- 5,21,25- triazatetracyclo[21.2.1.0^{2,7}.0^{19,24}]hexacosa- 1(26),2,4,6,16,23-hexaen-22-one |
| 70 | 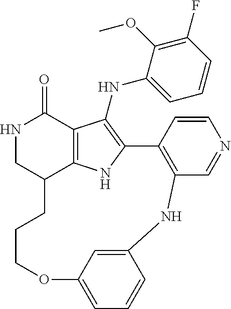 [SMILES: COc1c(F)cccc1Nc1c2[nH]c3c1C(=O)NCC3CCCOc1cccc(c1)Nc1cnccc1-2] | 26-[(3-fluoro-2-methoxyphenyl)amino]-20- oxa-2,5,10,14- tetraazapentacyclo[19.3.1.1^{9,12}.0^{3,8}.0^{11,16}]- hexacosa-1(25),3,5,7,9(26),11,21,23-octaen- 13-one |
| 71 | 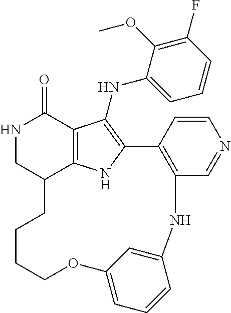 [SMILES: COc1c(F)cccc1Nc1c2[nH]c3c1C(=O)NCC3CCCCOc1cccc(c1)Nc1cnccc1-2] | 27-[(3-fluoro-2-methoxyphenyl)amino]-21- oxa-2,5,10,14- tetraazapentacyclo[20.3.1.1^{9,12}.0^{3,8}.0^{11,16}]- heptacosa-1(26),3,5,7,9(27),11,22,24-octaen- 13-one |
| 72 | 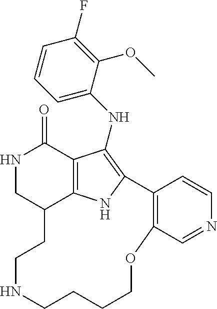 [SMILES: COc1c(F)cccc1Nc1c2[nH]c3c1C(=O)NCC3CCNCCCCOc1cnccc1-2] | 23-[(3-fluoro-2-methoxyphenyl)amino]-8- oxa-5,13,18,22- tetraazatetracyclo[18.2.1.0^{2,7}.0^{16,21}]tricosa- 1(23),2,4,6,20-pentaen-19-one |
| 73 | 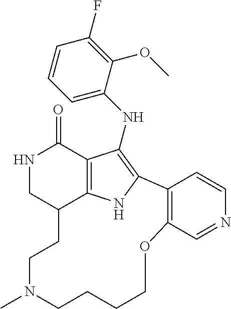 [SMILES: COc1c(F)cccc1Nc1c2[nH]c3c1C(=O)NCC3CCN(C)CCCCOc1cnccc1-2] | 23-[(3-fluoro-2-methoxyphenyl)amino]-13- methyl-8-oxa-5,13,18,22- tetraazatetracyclo[18.2.1.0^{2,7}.0^{16,21}]tricosa- 1(23),2,4,6,20-pentaen-19-one |
| 74 | 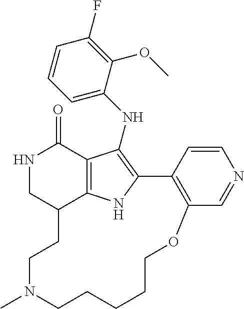 [SMILES: COc1c(F)cccc1Nc1c2[nH]c3c1C(=O)NCC3CCN(C)CCCCCOc1cnccc1-2] | 24-[(3-fluoro-2-methoxyphenyl)amino]-14- methyl-8-oxa-5,14,19,23- tetraazatetracyclo[19.2.1.0^{2,7}.0^{17,22}]tetracosa- 1(24),2,4,6,21-pentaen-20-one |
| 75 | 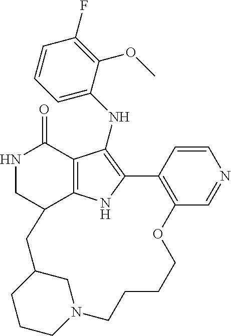 [SMILES: COc1c(F)cccc1Nc1c2[nH]c3c1C(=O)NCC3CC1CCCN(CCCCOc3cnccc3-2)C1] | 27-[(3-fluoro-2-methoxyphenyl)amino]-17- oxa-5,9,14,22- tetraazapentacyclo[20.3.1.1^{7,10}.0^{3,8}.0^{11,16}]- heptacosa-7,10(27),11,13,15-pentaen-6-one |
| 76 | 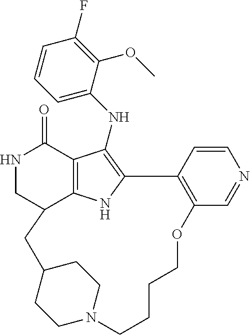 [SMILES: COc1c(F)cccc1Nc1c2[nH]c3c1C(=O)NCC3CC1CCN(CCCCOc3cnccc3-2)CC1] | 27-[(3-fluoro-2-methoxyphenyl)amino]-17- oxa-5,9,14,22- tetraazapentacyclo[20.2.2.1^{7,10}.0^{3,8}.0^{11,16}]- heptacosa-7,10(27),11,13,15-pentaen-6-one |
| 77 | 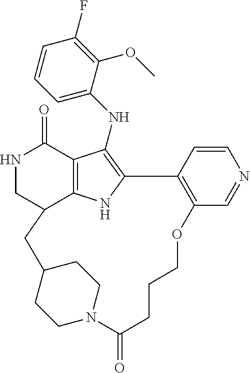 [SMILES: COc1c(F)cccc1Nc1c2[nH]c3c1C(=O)NCC3CC1CCN(CC1)C(=O)CCCOc1cnccc1-2] | 27-[(3-fluoro-2-methoxyphenyl)amino]-17- oxa-5,9,14,22- tetraazapentacyclo[20.2.2.1^{7,10}.0^{3,8}.0^{11,16}]- heptacosa-7,10(27),11,13,15-pentaene-6,21- dione |
| 78 | 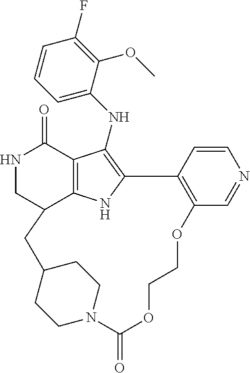 [SMILES: COc1c(F)cccc1Nc1c2[nH]c3c1C(=O)NCC3CC1CCN(CC1)C(=O)OCCOc1cnccc1-2] | 27-[(3-fluoro-2-methoxyphenyl)amino]- 17,20-dioxa-5,9,14,22- tetraazapentacyclo[20.2.2.1^{7,10}.0^{3,8}.0^{11,16}]- heptacosa-7,10(27),11,13,15-pentaene-6,21- dione |
| 79 | 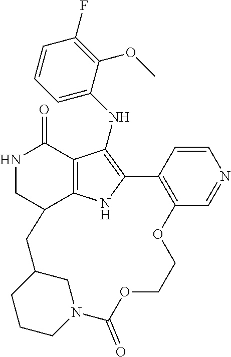 [SMILES: COc1c(F)cccc1Nc1c2[nH]c3c1C(=O)NCC3CC1CCCN(C1)C(=O)OCCOc1cnccc1-2] | 27-[(3-fluoro-2-methoxyphenyl)amino]- 17,20-dioxa-5,9,14,22- tetraazapentacyclo[20.3.1.1^{7,10}.0^{3,8}.0^{11,16}]hepta- cosa-7,10(27),11,13,15-pentaene-6,21-dione |
| 80 | 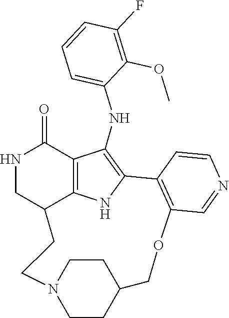 [SMILES: COc1c(F)cccc1Nc1c2[nH]c3c1C(=O)NCC3CCN1CCC(CC1)COc1cnccc1-2] | 25-[(3-fluoro-2-methoxyphenyl)amino]-18- oxa-1,6,10,15- tetraazapentacyclo[18.2.2.1^{8,11}.0^{4,9}.0^{12,17}]penta- cosa-8,11(25),12,14,16-pentaen-7-one |
| 81 | 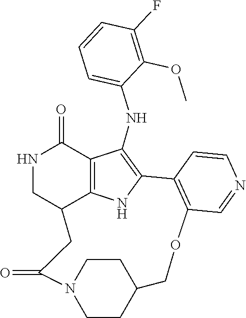 [SMILES: COc1c(F)cccc1Nc1c2[nH]c3c1C(=O)NCC3CC(=O)N1CCC(CC1)COc1cnccc1-2] | 25-[(3-fluoro-2-methoxyphenyl)amino]-18- oxa-1,6,10,15- tetraazapentacyclo[18.2.2.1^{8,11}.0^{4,9}.0^{12,17}]penta- cosa-8,11(25),12,14,16-pentaene-2,7-dione |
| 82 | 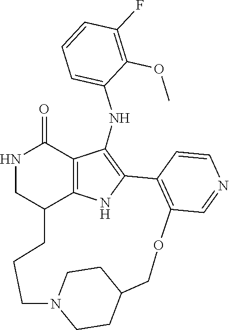 [SMILES: COc1c(F)cccc1Nc1c2[nH]c3c1C(=O)NCC3CCCN1CCC(CC1)COc1cnccc1-2] | 26-[(3-fluoro-2-methoxyphenyl)amino]-3- oxa-6,11,15,21- tetraazapentacyclo[19.2.2.1^{10,13}.0^{4,9}.0^{12,17}]- hexacosa-4,6,8,10(26),12-pentaen-14-one |
| 83 | 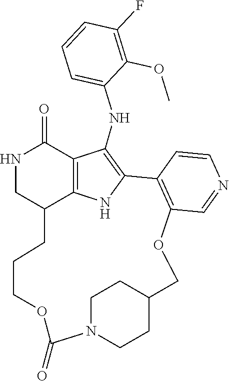 [SMILES: COc1c(F)cccc1Nc1c2[nH]c3c1C(=O)NCC3CCCOC(=O)N1CCC(CC1)COc1cnccc1-2] | 28-[(3-fluoro-2-methoxyphenyl)amino]-3,21- dioxa-6,11,15,23- tetraazapentacyclo[21.2.2.1^{10,13}.0^{4,9}.0^{12,17}]- octacosa-4,6,8,10(28),12-pentaene-14,22- dione |
| 84 | 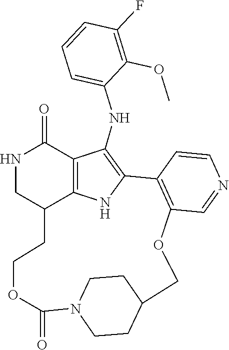 [SMILES: COc1c(F)cccc1Nc1c2[nH]c3c1C(=O)NCC3CCOC(=O)N1CCC(CC1)COc1cnccc1-2] | 27-[(3-fluoro-2-methoxyphenyl)amino]-3,20- dioxa-6,11,15,22- tetraazapentacyclo[20.2.2.1^{10,13}.0^{4,9}.0^{12,17}]- heptacosa-4,6,8,10(27),12-pentaene-14,21- dione |
| 85 | 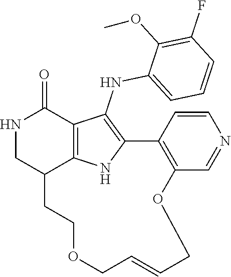 [SMILES: COc1c(F)cccc1Nc1c2[nH]c3c1C(=O)NCC3CCOC/C=C/COc1cnccc1-2] | (10E)-23-[(3-fluoro-2- methoxyphenyl)amino]-8,13-dioxa-5,18,22- triazatetracyclo[18.2.1.0^{2,7}.0^{16,21}]tricosa- 1(23),2,4,6,10,20-hexaen-19-one |
| 86 | 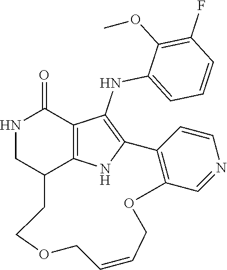 [SMILES: COc1c(F)cccc1Nc1c2[nH]c3c1C(=O)NCC3CCOC/C=C\COc1cnccc1-2] | (10Z)-23-[(3-fluoro-2- methoxyphenyl)amino]-8,13-dioxa-5,18,22- triazatetracyclo[18.2.1.0^{2,7}.0^{16,21}]tricosa- 1(23),2,4,6,10,20-hexaen-19-one |
| 87 | 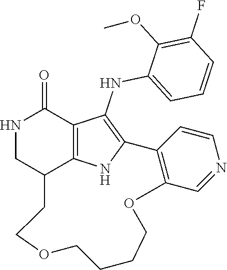 [SMILES: COc1c(F)cccc1Nc1c2[nH]c3c1C(=O)NCC3CCOCCCCOc1cnccc1-2] | 23-[(3-fluoro-2-methoxyphenyl)amino]-8,13- dioxa-5,18,22- triazatetracyclo[18.2.1.02,7.016,21]tricosa- 1(23),2,4,6,20-pentaen-19-one |
| 88 | 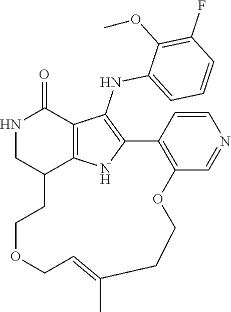 [SMILES: COc1c(F)cccc1Nc1c2[nH]c3c1C(=O)NCC3CCOC/C=C(\C)CCOc1cnccc1-2] | (11E)-24-[(3-fluoro-2- methoxyphenyl)amino]-11-methyl-8,14- dioxa-5,19,23- triazatetracyclo[19.2.1.0^{2,7}.0^{17,22}]tetracosa- 1(24),2,4,6,11,21-hexaen-20-one |
| 89 | 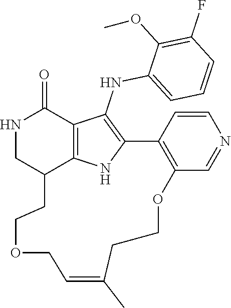 [SMILES: COc1c(F)cccc1Nc1c2[nH]c3c1C(=O)NCC3CCOC/C=C(/C)CCOc1cnccc1-2] | (11Z)-24-[(3-fluoro-2- methoxyphenyl)amino]-11-methyl-8,14- dioxa-5,19,23- triazatetracyclo[19.2.1.0^{2,7}.0^{17,22}]tetracosa- 1(24),2,4,6,11,21-hexaen-20-one |
| 90 | 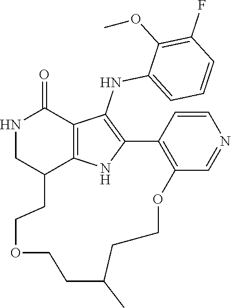 [SMILES: COc1c(F)cccc1Nc1c2[nH]c3c1C(=O)NCC3CCOCCC(C)CCOc1cnccc1-2] | 24-[(3-fluoro-2-methoxyphenyl)amino]-11- methyl-8,14-dioxa-5,19,23- triazatetracyclo[19.2.1.0^{2,7}.0^{17,22}]tetracosa- 1(24),2,4,6,21-pentaen-20-one |
| 91 | 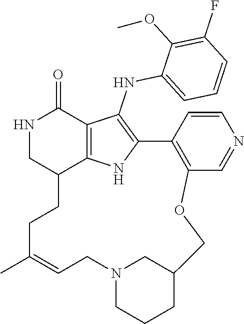 [SMILES: COc1c(F)cccc1Nc1c2[nH]c3c1C(=O)NCC3CC/C(C)=C\CN1CCCC(COc3cnccc3-2)C1] | (20Z)-28-[(3-fluoro-2- methoxyphenyl)amino]-20-methyl-3-oxa- 6,11,15,23- tetraazapentacyclo[21.3.1.1^{10,13}.0^{4,9}.0^{12,17}]octa- cosa-4,6,8,10(28),12,20-hexaen-14-one |
| 92 | 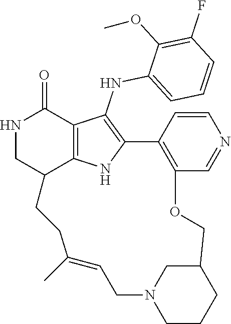 [SMILES: COc1c(F)cccc1Nc1c2[nH]c3c1C(=O)NCC3CC/C(C)=C/CN1CCCC(COc3cnccc3-2)C1] | (20E)-28-[(3-fluoro-2- methoxyphenyl)amino]-20-methyl-3-oxa- 6,11,15,23- tetraazapentacyclo[21.3.1.1^{10,13}.0^{4,9}.0^{12,17}]octa- cosa-4,6,8,10(28),12,20-hexaen-14-one |
| 93 | 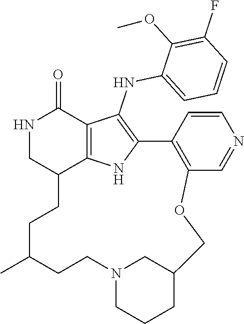 [SMILES: COc1c(F)cccc1Nc1c2[nH]c3c1C(=O)NCC3CCC(C)CCN1CCCC(COc3cnccc3-2)C1] | 28-[(3-fluoro-2-methoxyphenyl)amino]-20- methyl-3-oxa-6,11,15,23- tetraazapentacyclo[21.3.1.1^{10,13}.0^{4,9}.0^{12,17}]octa- cosa-4,6,8,10(28),12-pentaen-14-one 27-[(3-fluoro-2-methoxyphenyl)amino]-3- |
| 94 | 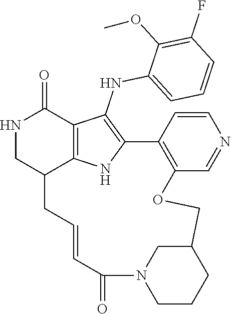 [SMILES: COc1c(F)cccc1Nc1c2[nH]c3c1C(=O)NCC3C/C=C/C(=O)N1CCCC(COc3cnccc3-2)C1] | oxa-6,11,15,22- tetraazapentacyclo[20.3.1.1^{10,13}.0^{4,9}.0^{12,17}]hept- acosa-4,6,8,10(27),12,19-hexaene-14,21- dione |
| 95 | 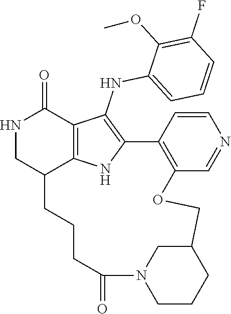 [SMILES: COc1c(F)cccc1Nc1c2[nH]c3c1C(=O)NCC3CCCC(=O)N1CCCC(COc3cnccc3-2)C1] | 27-[(3-fluoro-2-methoxyphenyl)amino]-3- oxa-6,11,15,22- tetraazapentacyclo[20.3.1.1^{10,13}.0^{4,9}.0^{12,17}]hept- acosa-4,6,8,10(27),12-pentaene-14,21-dione |
| 96 | 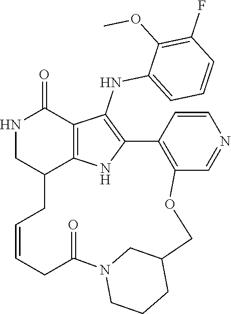 [SMILES: COc1c(F)cccc1Nc1c2[nH]c3c1C(=O)NCC3C/C=C\CC(=O)N1CCCC(COc3cnccc3-2)C1] | (19Z)-28-[(3-fluoro-2- methoxyphenyl)amino]-3-oxa-6,11,15,23- tetraazapentacyclo[21.3.1.1^{10,13}.0^{4,9}.0^{12,17}]octa- cosa-4,6,8,10(28),12,19-hexaene-14,22-dione |
| 97 | 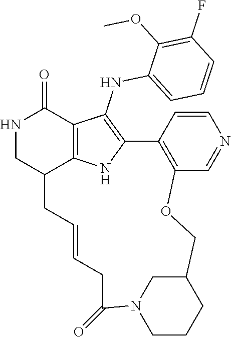 [SMILES: COc1c(F)cccc1Nc1c2[nH]c3c1C(=O)NCC3C/C=C/CC(=O)N1CCCC(COc3cnccc3-2)C1] | (19E)-28-[(3-fluoro-2- methoxyphenyl)amino]-3-oxa-6,11,15,23- tetraazapentacyclo[21.3.1.1^{10,13}.0^{4,9}.0^{12,17}]octa- cosa-4,6,8,10(28),12,19-hexaene-14,22-dione |
| 98 | 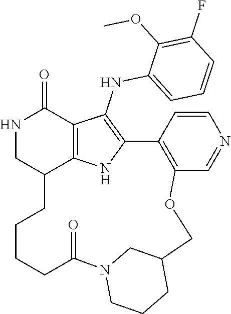 [SMILES: COc1c(F)cccc1Nc1c2[nH]c3c1C(=O)NCC3CCCCC(=O)N1CCCC(COc3cnccc3-2)C1] | 28-[(3-fluoro-2-methoxyphenyl)amino]-3- oxa-6,11,15,23- tetraazapentacyclo[21.3.1.1^{10,13}.0^{4,9}.0^{12,17}]octa- cosa-4,6,8,10(28),12-pentaene-14,22-dione |
| 99 | 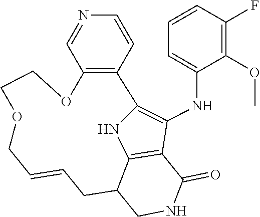 [SMILES: COc1c(F)cccc1Nc1c2[nH]c3c1C(=O)NCC3C/C=C/COCCOc1cnccc1-2] | (13E)-23-[(3-fluoro-2- methoxyphenyl)amino]-8,11-dioxa-5,18,22- triazatetracyclo[18.2.1.0^{2,7}.0^{16,21}]tricosa- 1(23),2,4,6,13,20-hexaen-19-one |
| 100 | 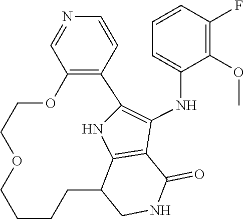 [SMILES: COc1c(F)cccc1Nc1c2[nH]c3c1C(=O)NCC3CCCCOCCOc1cnccc1-2] | 23-[(3-fluoro-2-methoxyphenyl)amino]-8,11- dioxa-5,18,22- triazatetracyclo[18.2.1.0^{2,7}.0^{16,21}]tricosa- 1(23),2,4,6,20-pentaen-19-one |
| 101 | 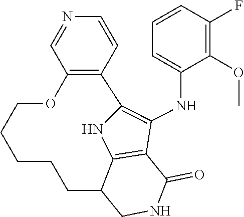 [SMILES: COc1c(F)cccc1Nc1c2[nH]c3c1C(=O)NCC3CCCCCOc1cnccc1-2] | 21-[(3-fluoro-2-methoxyphenyl)amino]-8- oxa-5,16,20- triazatetracyclo[16.2.1.0^{2,7}.0^{14,19}]henicosa- 1(21),2,4,6,18-pentaen-17-one |
| 102 | 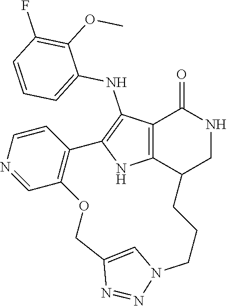 [SMILES: COc1c(F)cccc1Nc1c2[nH]c3c1C(=O)NCC3CCCn1cc(nn1)COc1cnccc1-2] | 24-[(3-fluoro-2-methoxyphenyl)amino]-8- oxa-5,11,12,13,19,23- hexaazapentacyclo[19.2.1.1^{10,13}.0^{2,7}.0^{17,22}]- pentacosa-1(24),2,4,6,10(25),11,21-heptaen- 20-one |
| 103 | 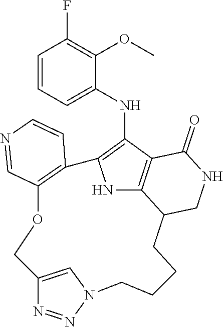 [SMILES: COc1c(F)cccc1Nc1c2[nH]c3c1C(=O)NCC3CCCCn1cc(nn1)COc1cnccc1-2] | 25-[(3-fluoro-2-methoxyphenyl)amino]-8- oxa-5,11,12,13,20,24- hexaazapentacyclo[20.2.1.1^{10,13}.0^{2,7}.0^{18,23}]- hexacosa-1(25),2,4,6,10(26),11,22-heptaen- 21-one |
| 104 | 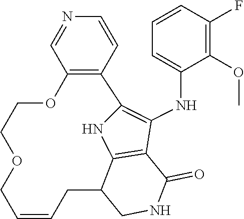 [SMILES: COc1c(F)cccc1Nc1c2[nH]c3c1C(=O)NCC3C/C=C\COCCOc1cnccc1-2] | (13Z)-23-[(3-fluoro-2- methoxyphenyl)amino]-8,11-dioxa-5,18,22- triazatetracyclo[18.2.1.0^{2,7}.0^{16,21}]tricosa- 1(23),2,4,6,13,20-hexaen-19-one |
| 105 | 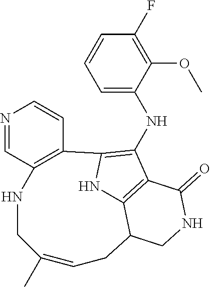 [SMILES: COc1c(F)cccc1Nc1c2[nH]c3c1C(=O)NCC3C/C=C(/C)CNc1cnccc1-2] | 20-[(3-fluoro-2-methoxyphenyl)amino]-10- methyl-5,8,15,19- tetraazatetracyclo[15.2.1.0^{2,7}.0^{13,18}]icosa- 1(20),2,4,6,10,17-hexaen-16-one |
| 106 | 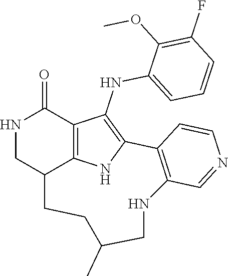 [SMILES: COc1c(F)cccc1Nc1c2[nH]c3c1C(=O)NCC3CCC(C)CNc1cnccc1-2] | 20-[(3-fluoro-2-methoxyphenyl)amino]-10- methyl-5,8,15,19- tetraazatetracyclo[15.2.1.0^{2,7}.0^{13,18}]icosa- 1(20),2,4,6,17-pentaen-16-one |
| 107 | 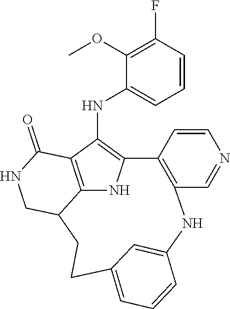 [SMILES: COc1c(F)cccc1Nc1c2[nH]c3c1C(=O)NCC3CCc1cccc(c1)Nc1cnccc1-2] | 24-[(3-fluoro-2-methoxyphenyl)amino]- 2,5,10,14- tetraazapentacyclo[17.3.1.1^{9,12}.0^{3,8}.0^{11,16}]- tetracosa-1(23),3,5,7,9(24),11,19,21-octaen- 13-one |
| 108 | 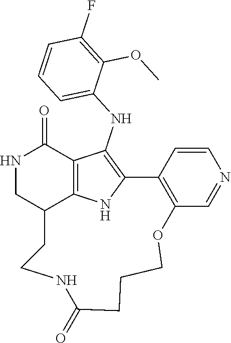 [SMILES: COc1c(F)cccc1Nc1c2[nH]c3c1C(=O)NCC3CCNC(=O)CCCOc1cnccc1-2] | 23-[(3-fluoro-2-methoxyphenyl)amino]-8- oxa-5,13,18,22- tetraazatetracyclo[18.2.1.0^{2,7}.0^{16,21}]tricosa- 1(23),2,4,6,20-pentaene-12,19-dione |
| 109 | 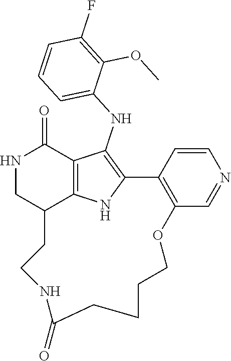 [SMILES: COc1c(F)cccc1Nc1c2[nH]c3c1C(=O)NCC3CCNC(=O)CCCCOc1cnccc1-2] | 24-[(3-fluoro-2-methoxyphenyl)amino]-8- oxa-5,14,19,23- tetraazatetracyclo[19.2.1.0^{2,7}.0^{17,22}]tetracosa- 1(24),2,4,6,21-pentaene-13,20-dione |
| 110 | 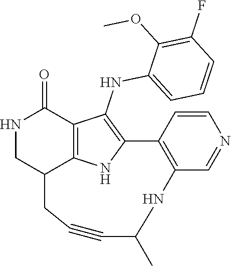 [SMILES: COc1c(F)cccc1Nc1c2[nH]c3c1C(=O)NCC3CC#CC(C)Nc1cnccc1-2] | 20-[(3-fluoro-2-methoxyphenyl)amino]-9- methyl-5,8,15,19- tetraazatetracyclo[15.2.1.0^{2,7}.0^{13,18}]icosa- 1(20),2,4,6,17-pentaen-10-yn-16-one |
| 111 | 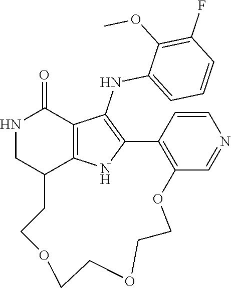 [SMILES: COc1c(F)cccc1Nc1c2[nH]c3c1C(=O)NCC3CCOCCOCCOc1cnccc1-2] | 24-[(3-fluoro-2-methoxyphenyl)amino]- 8,11,14-trioxa-5,19,23- triazatetracyclo[19.2.1.0^{2,7}.0^{17,22}]tetracosa- 1(24),2,4,6,21-pentaen-20-one |
| 112 | 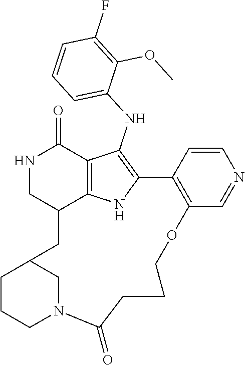 [SMILES: COc1c(F)cccc1Nc1c2[nH]c3c1C(=O)NCC3CC1CCCN(C1)C(=O)CCCOc1cnccc1-2] | 27-[(3-fluoro-2-methoxyphenyl)amino]-17- oxa-5,9,14,22- tetraazapentacyclo[20.3.1.1^{7,10}.0^{3,8}.0^{11,16}]- heptacosa-7,10(27),11,13,15-pentaene-6,21- dione |
| 113 | 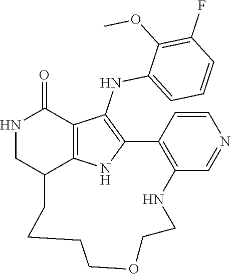 [SMILES: COc1c(F)cccc1Nc1c2[nH]c3c1C(=O)NCC3CCCCOCCNc1cnccc1-2] | 23-[(3-fluoro-2-methoxyphenyl)amino]-11- oxa-5,8,18,22- tetraazatetracyclo[18.2.1.0^{2,7}.0^{16,21}]tricosa- 1(23),2,4,6,20-pentaen-19-one |
| 114 | 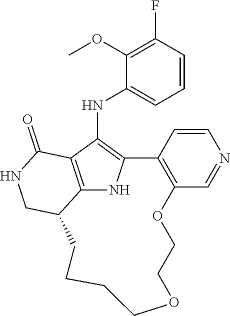 [SMILES: COc1c(F)cccc1Nc1c2[nH]c3c1C(=O)NC[C@H]3CCCCOCCOc1cnccc1-2] | (16R)-23-[(3-fluoro-2- methoxyphenyl)amino]-8,11-dioxa-5,18,22- triazatetracyclo[18.2.1.0^{2,7}.0^{16,21}]tricosa- 1(23),2,4,6,20-pentaen-19-one |
| 115 | 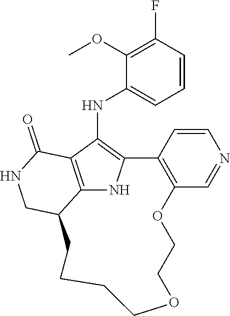 [SMILES: COc1c(F)cccc1Nc1c2[nH]c3c1C(=O)NC[C@@H]3CCCCOCCOc1cnccc1-2] | (16S)-23-[(3-fluoro-2- methoxyphenyl)amino]-8,11-dioxa-5,18,22- triazatetracyclo[18.2.1.0^{2,7}.0^{16,21}]tricosa- 1(23),2,4,6,20-pentaen-19-one |
| 116 | 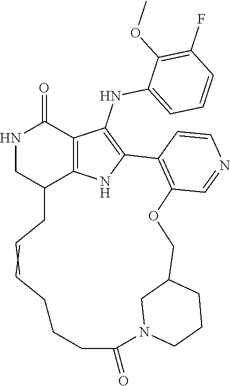 [SMILES: COc1c(F)cccc1Nc1c2[nH]c3c1C(=O)NCC3CC=CCCCC(=O)N1CCCC(COc3cnccc3-2)C1] | 25-[(3-fluoro-2-methoxyphenyl)amino]- 7,8,9,10,14,15,18,18a,19,20-decahydro-6H- 7,11-methano-22,24-methenodipyrido[3,4- b: 4′,3′-f][1,5,15]oxadiazacycloicosine- 12,21(13H,23H)-dione |
| 117 | 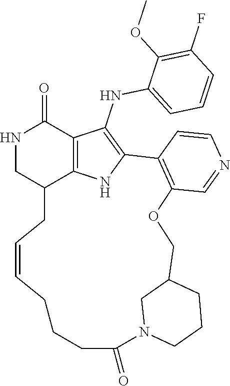 [SMILES: COc1c(F)cccc1Nc1c2[nH]c3c1C(=O)NCC3C/C=C\CCCC(=O)N1CCCC(COc3cnccc3-2)C1] | Z-25-[(3-fluoro-2-methoxyphenyl)amino]- 7,8,9,10,14,15,18,18a,19,20-decahydro-6H- 7,11-methano-22,24-methenodipyrido[3,4- b: 4′,3′-f][1,5,15]oxadiazacycloicosine- 12,21(13H,23H)-dione |
| 118 | 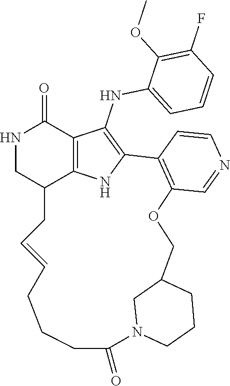 [SMILES: COc1c(F)cccc1Nc1c2[nH]c3c1C(=O)NCC3C/C=C/CCCC(=O)N1CCCC(COc3cnccc3-2)C1] | E-25-[(3-fluoro-2-methoxyphenyl)amino]- 7,8,9,10,14,15,18,18a,19,20-decahydro-6H- 7,11-methano-22,24-methenodipyrido[3,4- b: 4′,3′-f][1,5,15]oxadiazacycloicosine- 12,21(13H,23H)-dione |
| 119 | 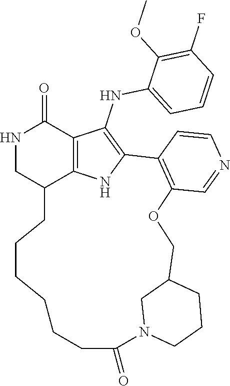 [SMILES: COc1c(F)cccc1Nc1c2[nH]c3c1C(=O)NCC3CCCCCCC(=O)N1CCCC(COc3cnccc3-2)C1] | 25-[(3-fluoro-2-methoxyphenyl)amino]- 7,8,9,10,14,15,16,17,18,18a,19,20- dodecahydro-6H-7,11-methano-22,24- methenodipyrido[3,4-b: 4′,3′- f][1,5,15]oxadiazacycloicosine- 12,21(13H,23H)-dione |
| 120 | 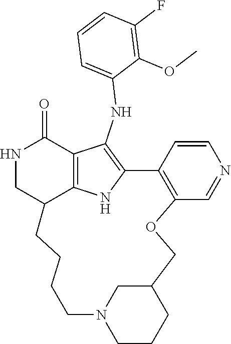 [SMILES: COc1c(F)cccc1Nc1c2[nH]c3c1C(=O)NCC3CCCCN1CCCC(COc3cnccc3-2)C1] | 22-(3-fluoro-2-methoxyanilino)- 7,8,9,10,13,14,15,15a,16,17-decahydro- 6H,12H-7,11-methano-19,21- (metheno)dipyrido[3,4-b: 4′,3′- f][1,5,12]oxadiazacycloheptadecin-18(20H)- one |
| 121 | 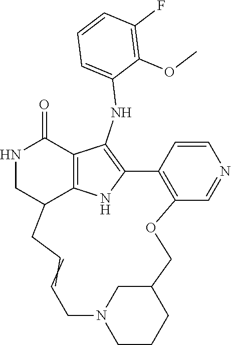 [SMILES: COc1c(F)cccc1Nc1c2[nH]c3c1C(=O)NCC3CC=CCN1CCCC(COc3cnccc3-2)C1] | 22-(3-fluoro-2-methoxyanilino)- 7,8,9,10,15,15a,16,17-octahydro-6H,12H- 7,11-methano-19,21-(metheno)dipyrido[3,4- b: 4′,3′-f][1,5,12]oxadiazacycloheptadecin- 18(20H)-one |
| 122 | 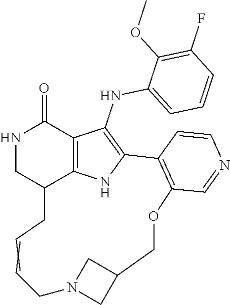 [SMILES: COc1c(F)cccc1Nc1c2[nH]c3c1C(=O)NCC3CC=CCN1CC(COc3cnccc3-2)C1] | 20-(3-fluoro-2-methoxyanilino)- 7,8,13,13a,14,15-hexahydro-6H,10H-7,9- methano-17,19-(metheno)dipyrido[3,4-b: 4′,3′- f][1,5,12]oxadiazacyclopentadecin-16(18H)- one |
| 123 | 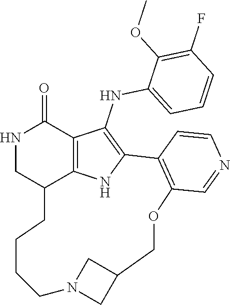 [SMILES: COc1c(F)cccc1Nc1c2[nH]c3c1C(=O)NCC3CCCCN1CC(COc3cnccc3-2)C1] | 20-(3-fluoro-2-methoxyanilino)- 7,8,11,12,13,13a,14,15-octahydro-6H,10H- 7,9-methano-17,19-(metheno)dipyrido[3,4- b: 4′,3′-f][1,5,12]oxadiazacyclopentadecin- 16(18H)-one |
| 124 | 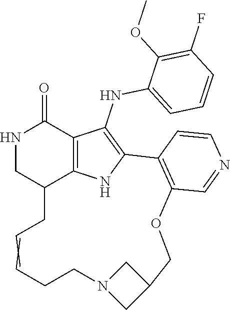 [SMILES: COc1c(F)cccc1Nc1c2[nH]c3c1C(=O)NCC3CC=CCCN1CC(COc3cnccc3-2)C1] | 21-(3-fluoro-2-methoxyanilino)- 7,8,10,11,14,14a,15,16-octahydro-6H-7,9- methano-18,20-(metheno)dipyrido[3,4-b: 4′,3′- f][1,5,13]oxadiazacyclohexadecin-17(19H)- one |
| 125 | 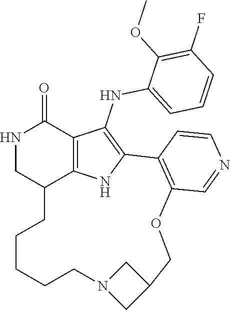 [SMILES: COc1c(F)cccc1Nc1c2[nH]c3c1C(=O)NCC3CCCCCN1CC(COc3cnccc3-2)C1] | 21-(3-fluoro-2-methoxyanilino)- 7,8,10,11,12,13,14,14a,15,16-decahydro-6H- 7,9-methano-18,20-(metheno)dipyrido[3,4- b: 4′,3′-f][1,5,13]oxadiazacyclohexadecin- 17(19H)-one |
| 126 | 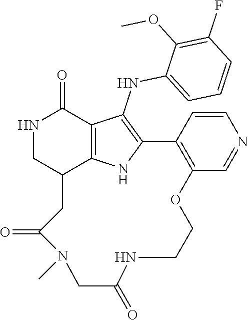 [SMILES: COc1c(F)cccc1Nc1c2[nH]c3c1C(=O)NCC3CC(=O)N(C)CC(=O)NCCOc1cnccc1-2] | 20-(3-fluoro-2-methoxyanilino)-11-methyl- 7,8,10,11,13,13a,14,15-octahydro-6H-17,19- (metheno)dipyrido[3,4-j: 4′,3′- n][1,4,7,12]oxatriazacyclopentadecine- 9,12,16(18H)-trione |
| 127 | 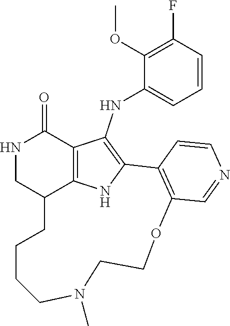 [SMILES: COc1c(F)cccc1Nc1c2[nH]c3c1C(=O)NCC3CCCCN(C)CCOc1cnccc1-2] | 19-(3-fluoro-2-methoxyanilino)-8-methyl- 6,7,8,9,10,11,12,12a,13,14-decahydro-16,18- (metheno)dipyrido[3,4-i: 4′,3′- m][1,4,11]oxadiazacyclotetradecin-15(17H)- one |
| 128 | 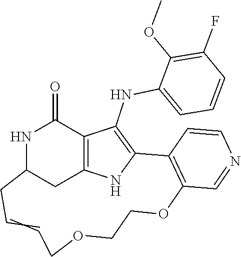 [SMILES: COc1c(F)cccc1Nc1c2[nH]c3c1C(=O)NC(CC=CCOCCOc1cnccc1-2)C3] | 20-(3-fluoro-2-methoxyanilino)-2,3,5,8,9,10- hexahydro-9,13-methano-12,15- (metheno)pyrido[3,4- e][1,4,8,12]dioxadiazacycloheptadecin- 11(14H)-one |
| 129 | 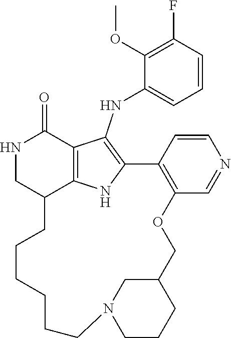 [SMILES: COc1c(F)cccc1Nc1c2[nH]c3c1C(=O)NCC3CCCCCCN1CCCC(COc3cnccc3-2)C1] | 24-(3-fluoro-2-methoxyanilino)- 7,8,9,10,13,14,15,16,17,17a,18,19- dodecahydro-6H,12H-7,11-methano-21,23- (metheno)dipyrido[3,4-b: 4′,3′- f][1,5,14]oxadiazacyclononadecin-20(22H)- one |
| 130 | 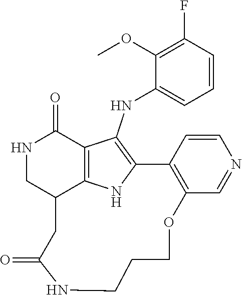 [SMILES: COc1c(F)cccc1Nc1c2[nH]c3c1C(=O)NCC3CC(=O)NCCCOc1cnccc1-2] | 18-(3-fluoro-2-methoxyanilino)- 8,9,11,11a,12,13-hexahydro-6H-15,17- (metheno)dipyrido[3,4-b: 4′,3′- f][1,5,10]oxadiazacyclotridecine- 10,14(7H,16H)-dione |
| 131 | 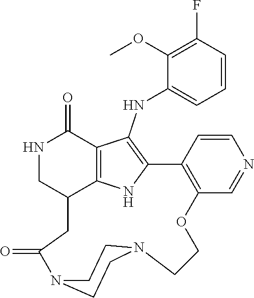 [SMILES: COc1c(F)cccc1Nc1c2[nH]c3c1C(=O)NCC3CC(=O)N1CCN(CCOc3cnccc3-2)CC1] | 20-(3-fluoro-2-methoxyanilino)- 6,7,9,10,13,13a,14,15-octahydro-12H-8,11- ethano-17,19-(metheno)dipyrido[3,4-j: 4′,3′- n][1,4,7,12]oxatriazacyclopentadecine- 12,16(18H)-dione |
| 132 | 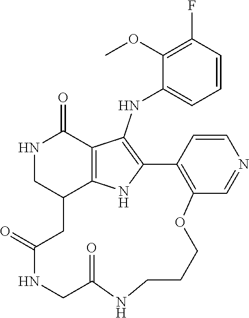 [SMILES: COc1c(F)cccc1Nc1c2[nH]c3c1C(=O)NCC3CC(=O)NCC(=O)NCCCOc1cnccc1-2] | 21-(3-fluoro-2-methoxyanilino)- 6,7,8,9,11,12,14,14a,15,16-decahydro-18,20- (metheno)dipyrido[3,4-k: 4′,3′- o][1,5,8,13]oxatriazacyclohexadecine- 10,13,17(19H)-trione |
| 133 | 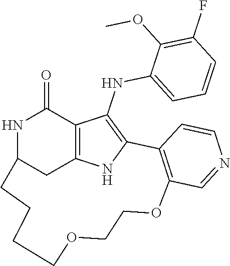 [SMILES: COc1c(F)cccc1Nc1c2[nH]c3c1C(=O)NC(CCCCOCCOc1cnccc1-2)C3] | 20-(3-fluoro-2-methoxyanilino)- 2,3,5,6,7,8,9,10-octahydro-9,13-methano- 12,15-(metheno)pyrido[3,4- e][1,4,8,12]dioxadiazacycloheptadecin- 11(14H)-one |
| 134 | 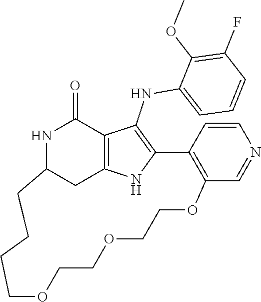 [SMILES: COc1c(F)cccc1Nc1c2[nH]c3c1C(=O)NC(CCCCOCCOCCOc1cnccc1-2)C3] | 23-(3-fluoro-2-methoxyanilino)- 2,3,5,6,8,9,10,11,12,13-decahydro-12,16- methano-15,18-(metheno)pyrido[3,4- h][1,4,7,11,15]trioxadiazacycloicosin- 14(17H)-one |
| 135 | 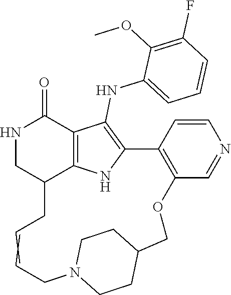 [SMILES: COc1c(F)cccc1Nc1c2[nH]c3c1C(=O)NCC3CC=CCN1CCC(CC1)COc1cnccc1-2] | 21-(3-fluoro-2-methoxyanilino)- 6,7,8,9,14,14a,15,16-octahydro-11H-7,10- ethano-18,20-(metheno)dipyrido[3,4-b: 4′,3′- f][1,5,12]oxadiazacyclohexadecin-17(19H)- one |
| 136 | 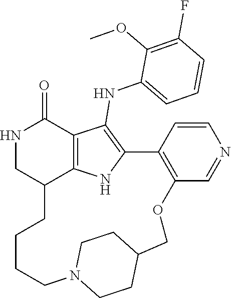 [SMILES: COc1c(F)cccc1Nc1c2[nH]c3c1C(=O)NCC3CCCCN1CCC(CC1)COc1cnccc1-2] | 21-(3-fluoro-2-methoxyanilino)- 6,7,8,9,12,13,14,14a,15,16-decahydro-11H- 7,10-ethano-18,20-(metheno)dipyrido[3,4- b: 4′,3′-f][1,5,12]oxadiazacyclohexadecin- 17(19H)-one |
| 137 | 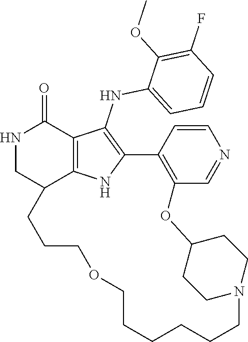 [SMILES: COc1c(F)cccc1Nc1c2[nH]c3c1C(=O)NCC3CCCOCCCCCCN1CCC(CC1)Oc1cnccc1-2] | 28-(3-fluoro-2-methoxyanilino)- 1,2,13,14,16,17,18,19,20,21,23,24,25,25a- tetradecahydro-12H-12,15-ethano-4,6- (metheno)dipyrido[3,4-b: 4′,3′- f][1,11,5,18]dioxadiazacyclohenicosin-3(5H)- one |
| 138 | 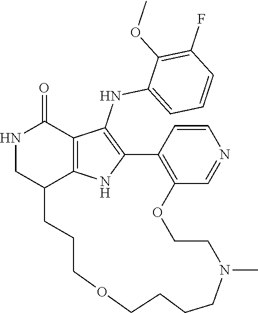 [SMILES: COc1c(F)cccc1Nc1c2[nH]c3c1C(=O)NCC3CCCOCCCCN(C)CCOc1cnccc1-2] | 23-(3-fluoro-2-methoxyanilino)-8-methyl- 7,8,9,10,11,12,14,15,16,16a,17,18- dodecahydro-6H-20,22- (metheno)dipyrido[3,4-m: 4′,3′- q][1,9,4,15]dioxadiazacyclooctadecin- 19(21H)-one |
| 139 | 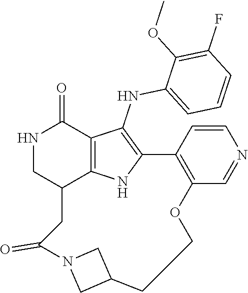 [SMILES: COc1c(F)cccc1Nc1c2[nH]c3c1C(=O)NCC3CC(=O)N1CC(CCOc3cnccc3-2)C1] | 19-(3-fluoro-2-methoxyanilino)- 6,7,8,9,12,12a,13,14-octahydro-11H-8,10- methano-16,18-(metheno)dipyrido[3,4-b: 4′,3′- f][1,5,10]oxadiazacyclotetradecine- 11,15(17H)-dione |
| 140 | 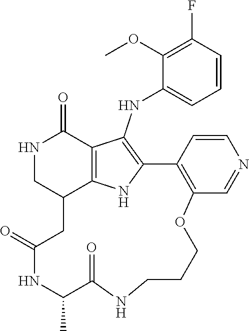 [SMILES: COc1c(F)cccc1Nc1c2[nH]c3c1C(=O)NCC3CC(=O)N[C@@H](C)C(=O)NCCCOc1cnccc1-2] | (11S)-21-(3-fluoro-2-methoxyanilino)-11- methyl-6,7,8,9,11,12,14,14a,15,16- decahydro-18,20-(metheno)dipyrido[3,4- k: 4′,3′-o][1,5,8,13]oxatriazacyclohexadecine- 10,13,17(19H)-trione |
| 141 | 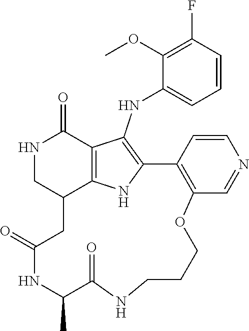 [SMILES: COc1c(F)cccc1Nc1c2[nH]c3c1C(=O)NCC3CC(=O)N[C@H](C)C(=O)NCCCOc1cnccc1-2] | (11R)-21-(3-fluoro-2-methoxyanilino)-11- methyl-6,7,8,9,11,12,14,14a,15,16- decahydro-18,20-(metheno)dipyrido[3,4- k: 4′,3′-o][1,5,8,13]oxatriazacyclohexadecine- 10,13,17(19H)-trione |
| 142 | 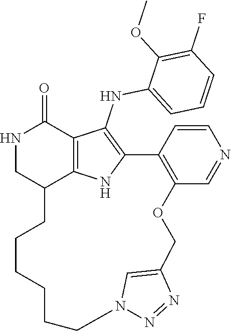 [SMILES: COc1c(F)cccc1Nc1c2[nH]c3c1C(=O)NCC3CCCCCCn1cc(nn1)COc1cnccc1-2] | 23-(3-fluoro-2-methoxyanilino)- 12,13,14,15,16,16a,17,18-octahydro-6H,11H- 10,7: 20,22-di(metheno)dipyrido[3,4-m: 4′,3′- q][1,4,5,6,15]oxatetraazacyclooctadecin- 19(21H)-one |
| 143 | 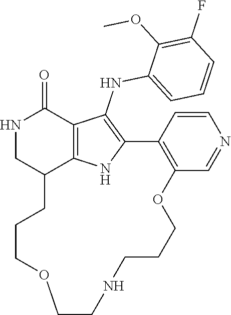 [SMILES: COc1c(F)cccc1Nc1c2[nH]c3c1C(=O)NCC3CCCOCCNCCCOc1cnccc1-2] | 22-(3-fluoro-2-methoxyanilino)- 6,7,8,9,10,11,13,14,15,15a,16,17- dodecahydro-19,21-(metheno)dipyrido[3,4- i: 4′,3′- m][1,8,4,12]dioxadiazacycloheptadecin- 18(20H)-one |
| 144 | 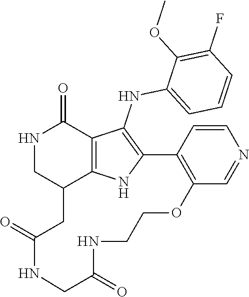 [SMILES: COc1c(F)cccc1Nc1c2[nH]c3c1C(=O)NCC3CC(=O)NCC(=O)NCCOc1cnccc1-2] | 20-(3-fluoro-2-methoxyanilino)- 7,8,10,11,13,13a,14,15-octahydro-6H-17,19- (metheno)dipyrido[3,4-j: 4′,3′- n][1,4,7,12]oxatriazacyclopentadecine- 9,12,16(18H)-trione |
| 145 | 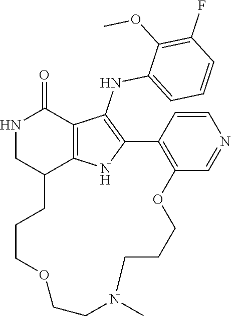 [SMILES: COc1c(F)cccc1Nc1c2[nH]c3c1C(=O)NCC3CCCOCCN(C)CCCOc1cnccc1-2] | 22-(3-fluoro-2-methoxyanilino)-9-methyl- 6,7,8,9,10,11,13,14,15,15a,16,17- dodecahydro-19,21-(metheno)dipyrido[3,4- i: 4′,3′- m][1,8,4,12]dioxadiazacycloheptadecin- 18(20H)-one |
| 146 | 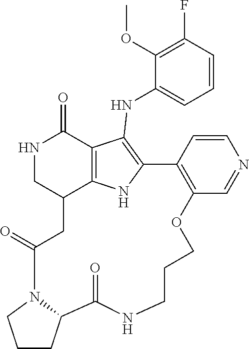 [SMILES: COc1c(F)cccc1Nc1c2[nH]c3c1C(=O)NCC3CC(=O)N1CCC[C@H]1C(=O)NCCCOc1cnccc1-2] | (16aS)-23-(3-fluoro-2-methoxyanilino)- 14,15,16a,17,18,19,22,22a-octahydro- 2H,12H-4,6-(metheno)dipyrido[3,4-k: 4′,3′- o]pyrrolo[2,1- g][1,5,8,13]oxatriazacyclohexadecine- 3,16,21(1H,5H,13H)-trione |
| 147 | 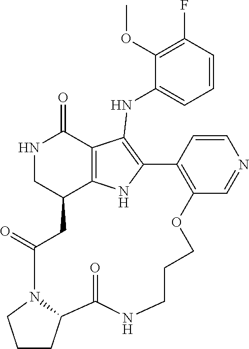 [SMILES: COc1c(F)cccc1Nc1c2[nH]c3c1C(=O)NC[C@@H]3CC(=O)N1CCC[C@H]1C(=O)NCCCOc1cnccc1-2] | (16aS,22aS)-23-(3-fluoro-2-methoxyanilino)- 14,15,16a,17,18,19,22,22a-octahydro- 2H,12H-4,6-(metheno)dipyrido[3,4-k: 4′,3′- o]pyrrolo[2,1- g][1,5,8,13]oxatriazacyclohexadecine- 3,16,21(1H,5H,13H)-trione |
| 148 | 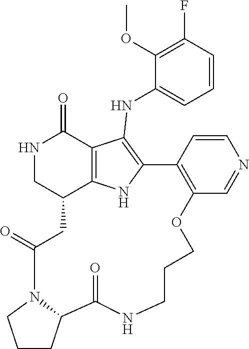 [SMILES: COc1c(F)cccc1Nc1c2[nH]c3c1C(=O)NC[C@H]3CC(=O)N1CCC[C@H]1C(=O)NCCCOc1cnccc1-2] | (16aS,22aR)-23-(3-fluoro-2-methoxyanilino)- 14,15,16a,17,18,19,22,22a-octahydro- 2H,12H-4,6-(metheno)dipyrido[3,4-k: 4′,3′- o]pyrrolo[2,1- g][1,5,8,13]oxatriazacyclohexadecine- 3,16,21(1H,5H,13H)-trione |
| 149 | 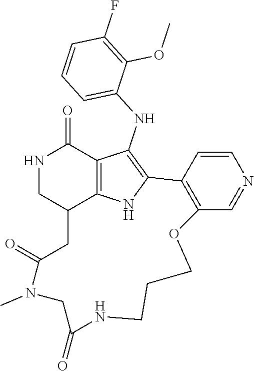 [SMILES: COc1c(F)cccc1Nc1c2[nH]c3c1C(=O)NCC3CC(=O)N(C)CC(=O)NCCCOc1cnccc1-2] | 21-(3-fluoro-2-methoxyanilino)-12-methyl- 6,7,8,9,11,12,14,14a,15,16-decahydro-18,20- (metheno)dipyrido[3,4-k: 4′,3′- o][1,5,8,13]oxatriazacyclohexadecine- 10,13,17(19H)-trione |
| 150 | 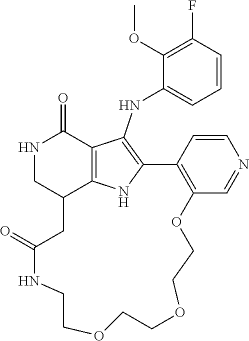 [SMILES: COc1c(F)cccc1Nc1c2[nH]c3c1C(=O)NCC3CC(=O)NCCOCCOCCOc1cnccc1-2] | 23-(3-fluoro-2-methoxyanilino)- 6,7,9,10,13,14,16,16a,17,18-decahydro- 20,22-(metheno)dipyrido[3,4-m: 4′,3′- q][1,4,7,10,15]trioxadiazacyclooctadecine- 15,19(12H,21H)-dione |
| 151 | 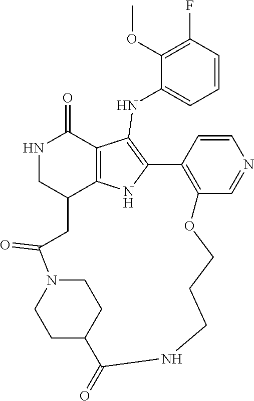 [SMILES: COc1c(F)cccc1Nc1c2[nH]c3c1C(=O)NCC3CC(=O)N1CCC(CC1)C(=O)NCCCOc1cnccc1-2] | 23-(3-fluoro-2-methoxyanilino)- 6,7,8,9,12,13,16,16a,17,18-decahydro-15H- 11,14-ethano-20,22-(metheno)dipyrido[3,4- b: 4′,3′-f][1,5,10,15]oxatriazacyclooctadecine- 10,15,19(11H,21H)-trione |
| 152 | 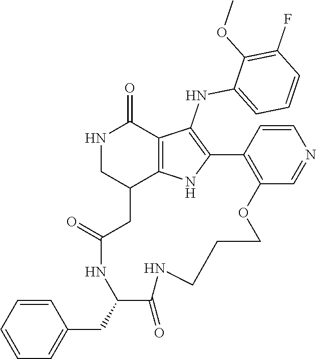 [SMILES: COc1c(F)cccc1Nc1c2[nH]c3c1C(=O)NCC3CC(=O)N[C@@H](Cc1ccccc1)C(=O)NCCCOc1cnccc1-2] | (11S)-11-benzyl-21-(3-fluoro-2- methoxyanilino)-6,7,8,9,11,12,14,14a,15,16- decahydro-18,20-(metheno)dipyrido[3,4- k: 4′,3′-o][1,5,8,13]oxatriazacyclohexadecine- 10,13,17(19H)-trione |
| 153 | 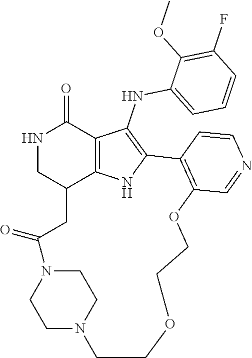 [SMILES: COc1c(F)cccc1Nc1c2[nH]c3c1C(=O)NCC3CC(=O)N1CCN(CCOCCOc3cnccc3-2)CC1] | 23-(3-fluoro-2-methoxyanilino)- 6,7,9,10,12,13,16,16a,17,18-decahydro-15H- 11,14-ethano-20,22-(metheno)dipyrido[3,4- m: 4′,3′- q][1,4,7,10,15]dioxatriazacyclooctadecine- 15,19(21H)-dione trifluoroacetic acid (1\1) |
| 154 | 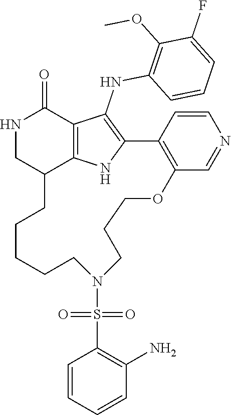 [SMILES: COc1c(F)cccc1Nc1c2[nH]c3c1C(=O)NCC3CCCCCN(S(=O)(=O)c1ccccc1N)CCCOc1cnccc1-2] | 9-(2-aminobenzene-1-sulfonyl)-21-(3-fluoro- 2-methoxyanilino)- 6,7,8,9,10,11,12,13,14,14a,15,16- dodecahydro-18,20-(metheno)dipyrido[3,4- b: 4′,3′-f][1,5,13]oxadiazacyclohexadecin- 17(19H)-one |
| 155 | 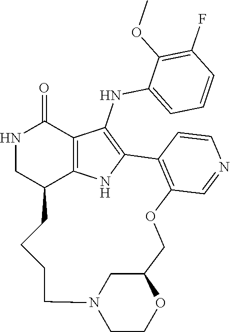 [SMILES: COc1c(F)cccc1Nc1c2[nH]c3c1C(=O)NC[C@@H]3CCCCN1CCO[C@H](COc3cnccc3-2)C1] | (7S,15aS)-22-(3-fluoro-2-methoxyanilino)- 6,7,9,10,13,14,15,15a,16,17-decahydro-12H- 7,11-methano-19,21-(metheno)dipyrido[3,4- 1: 4′,3′-p][1,4,7,14]dioxadiazacycloheptadecin- 18(20H)-one |
| 156 | 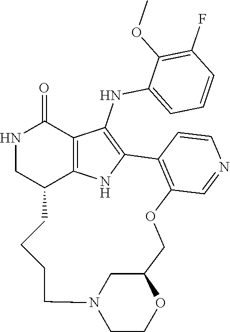 [SMILES: COc1c(F)cccc1Nc1c2[nH]c3c1C(=O)NC[C@H]3CCCCN1CCO[C@H](COc3cnccc3-2)C1] | (7S,15aR)-22-(3-fluoro-2-methoxyanilino)- 6,7,9,10,13,14,15,15a,16,17-decahydro-12H- 7,11-methano-19,21-(metheno)dipyrido[3,4- 1: 4′,3′-p][1,4,7,14]dioxadiazacycloheptadecin- 18(20H)-one |
| 157 | 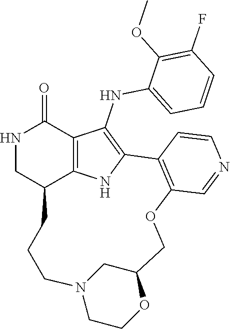 [SMILES: COc1c(F)cccc1Nc1c2[nH]c3c1C(=O)NC[C@@H]3CCCN1CCO[C@H](COc3cnccc3-2)C1] | (7S,14aS)-21-(3-fluoro-2-methoxyanilino)- 6,7,9,10,12,13,14,14a,15,16-decahydro-7,11- methano-18,20-(metheno)dipyrido[3,4-k: 4′,3′- o][1,4,7,13]dioxadiazacyclohexadecin- 17(19H)-one |
| 158 | 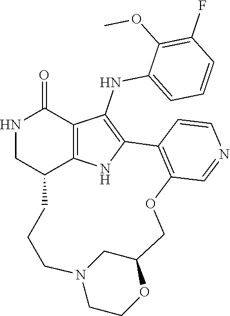 [SMILES: COc1c(F)cccc1Nc1c2[nH]c3c1C(=O)NC[C@H]3CCCN1CCO[C@H](COc3cnccc3-2)C1] | (7S,14aR)-21-(3-fluoro-2-methoxyanilino)- 6,7,9,10,12,13,14,14a,15,16-decahydro-7,11- methano-18,20-(metheno)dipyrido[3,4-k: 4′,3′- o][1,4,7,13]dioxadiazacyclohexadecin- 17(19H)-one |
| 159 | 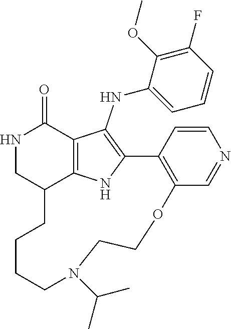 [SMILES: COc1c(F)cccc1Nc1c2[nH]c3c1C(=O)NCC3CCCCN(C(C)C)CCOc1cnccc1-2] | 19-(3-fluoro-2-methoxyanilino)-8-(propan-2- yl)-6,7,8,9,10,11,12,12a,13,14-decahydro- 16,18-(metheno)dipyrido[3,4-i: 4′,3′- m][1,4,11]oxadiazacyclotetradecin-15(17H)- one |
| 160 | 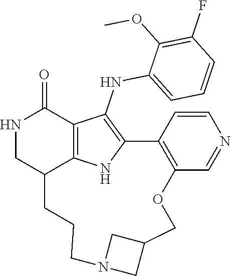 [SMILES: COc1c(F)cccc1Nc1c2[nH]c3c1C(=O)NCC3CCCN1CC(COc3cnccc3-2)C1] | 19-(3-fluoro-2-methoxyanilino)- 7,8,10,11,12,12a,13,14-octahydro-6H-7,9- methano-16,18-(metheno)dipyrido[3,4-b: 4′,3′- f][1,5,11]oxadiazacyclotetradecin-15(17H)- one |
| 161 | 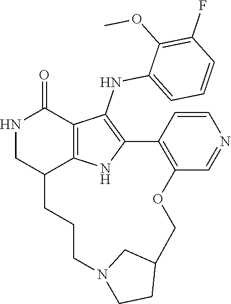 [SMILES: COc1c(F)cccc1Nc1c2[nH]c3c1C(=O)NCC3CCCN1CCC(COc3cnccc3-2)C1] | 20-(3-fluoro-2-methoxyanilino)- 6,7,8,9,11,12,13,13a,14,15-decahydro-7,10- methano-17,19-(metheno)dipyrido[3,4-b: 4′,3′- f][1,5,11]oxadiazacyclopentadecin-16(18H)- one |
| 162 | 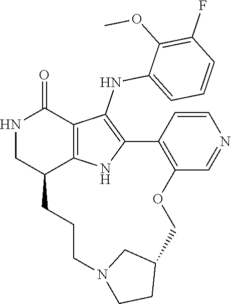 [SMILES: COc1c(F)cccc1Nc1c2[nH]c3c1C(=O)NC[C@@H]3CCCN1CC[C@@H](COc3cnccc3-2)C1] | (7R,13aS)-20-(3-fluoro-2-methoxyanilino)- 6,7,8,9,11,12,13,13a,14,15-decahydro-7,10- methano-17,19-(metheno)dipyrido[3,4-b: 4′,3′- f][1,5,11]oxadiazacyclopentadecin-16(18H)- one |
| 163 | 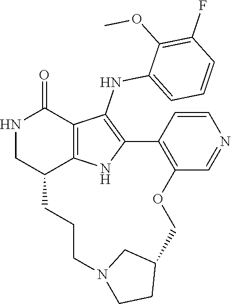 [SMILES: COc1c(F)cccc1Nc1c2[nH]c3c1C(=O)NC[C@H]3CCCN1CC[C@@H](COc3cnccc3-2)C1] | (7R,13aR)-20-(3-fluoro-2-methoxyanilino)- 6,7,8,9,11,12,13,13a,14,15-decahydro-7,10- methano-17,19-(metheno)dipyrido[3,4-b: 4′,3′- f][1,5,11]oxadiazacyclopentadecin-16(18H)- one |
| 164 | 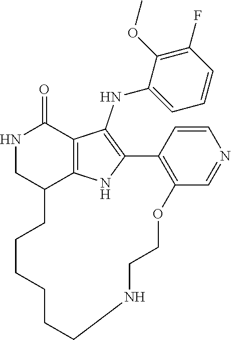 [SMILES: COc1c(F)cccc1Nc1c2[nH]c3c1C(=O)NCC3CCCCCCNCCOc1cnccc1-2] | 21-(3-fluoro-2-methoxyanilino)- 6,7,8,9,10,11,12,13,14,14a,15,16- dodecahydro-18,20-(metheno)dipyrido[3,4- k: 4′,3′-o][1,4,13]oxadiazacyclohexadecin- 17(19H)-one |
| 165 | 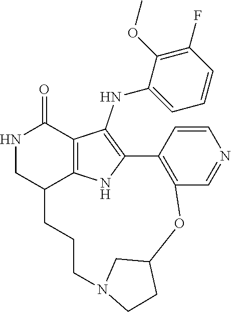 [SMILES: COc1c(F)cccc1Nc1c2[nH]c3c1C(=O)NCC3CCCN1CCC(C1)Oc1cnccc1-2] | 19-(3-fluoro-2-methoxyanilino)- 7,8,10,11,12,12a,13,14-octahydro-6H-6,9- methano-16,18-(metheno)dipyrido[3,4-b: 4′,3′- f][1,5,11]oxadiazacyclotetradecin-15(17H)- one |
| 166 | 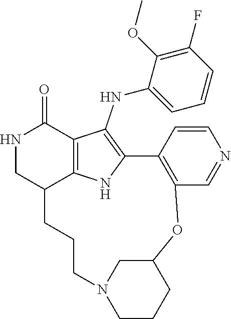 [SMILES: COc1c(F)cccc1Nc1c2[nH]c3c1C(=O)NCC3CCCN1CCCC(C1)Oc1cnccc1-2] | 20-(3-fluoro-2-methoxyanilino)- 6,7,8,9,11,12,13,13a,14,15-decahydro-6,10- methano-17,19-(metheno)dipyrido[3,4-b: 4′,3′- f][1,5,11]oxadiazacyclopentadecin-16(18H)- one |
| 167 | 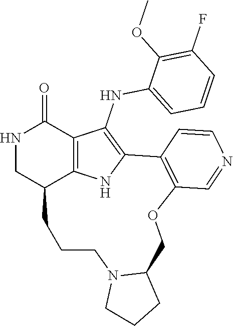 [SMILES: COc1c(F)cccc1Nc1c2[nH]c3c1C(=O)NC[C@@H]3CCCN1CCC[C@@H]1COc1cnccc1-2] | (12aR,19aS)-20-(3-fluoro-2-methoxyanilino)- 1,2,12a,13,14,15,17,18,19,19a-decahydro- 12H-4,6-(metheno)dipyrido[3,4-h: 4′,3′- l]pyrrolo[2,1-c][1,4,10]oxadiazacyclotridecin- 3(5H)-one |
| 168 | 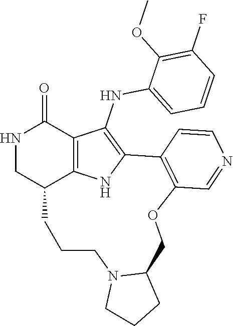 [SMILES: COc1c(F)cccc1Nc1c2[nH]c3c1C(=O)NC[C@H]3CCCN1CCC[C@@H]1COc1cnccc1-2] | (12aR,19aR)-20-(3-fluoro-2-methoxyanilino)- 1,2,12a,13,14,15,17,18,19,19a-decahydro- 12H-4,6-(metheno)dipyrido[3,4-h: 4′,3′- l]pyrrolo[2,1-c][1,4,10]oxadiazacyclotridecin- 3(5H)-one |
| 169 | 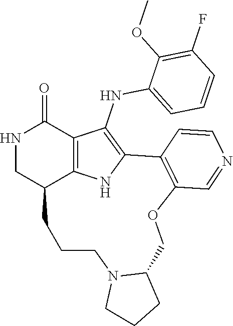 [SMILES: COc1c(F)cccc1Nc1c2[nH]c3c1C(=O)NC[C@@H]3CCCN1CCC[C@H]1COc1cnccc1-2] | (12aS,19aS)-20-(3-fluoro-2-methoxyanilino)- 1,2,12a,13,14,15,17,18,19,19a-decahydro- 12H-4,6-(metheno)dipyrido[3,4-h: 4′,3′- l]pyrrolo[2,1-c][1,4,10]oxadiazacyclotridecin- 3(5H)-one |
| 170 | 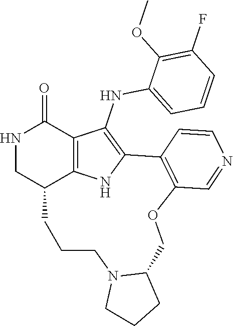 [SMILES: COc1c(F)cccc1Nc1c2[nH]c3c1C(=O)NC[C@H]3CCCN1CCC[C@H]1COc1cnccc1-2] | (12aS,19aR)-20-(3-fluoro-2-methoxyanilino)- 1,2,12a,13,14,15,17,18,19,19a-decahydro- 12H-4,6-(metheno)dipyrido[3,4-h: 4′,3′- l]pyrrolo[2,1-c][1,4,10]oxadiazacyclotridecin- 3(5H)-one |

In some embodiments, the compound is a pharmaceutically acceptable salt of any one of the compounds described in Table 3.

In some embodiments, the compound is a lithium salt, sodium salt, potassium salt, calcium salt, or magnesium salt of any one of the compounds described in Table 3.

In some embodiments, the compound is a salt of any acid described in the Table 4 and any one of the compounds described in Table 3.

TABLE 4
Pharmaceutical acceptable acid forming
salts with the Compound of Formula (A).

1-hydroxy-2-naphthoic acid;
2,2-dichloroacetic acid;
2-hydroxyethanesulfonic acid;
2-oxoglutaric acid;
4-acetamidobenzoic acid;
4-aminosalicylic acid;
acetic acid;
adipic acid;
ascorbic acid (L);
aspartic acid (L);
benzenesulfonic acid;
benzoic acid;
camphoric acid (+);
camphor-10-sulfonic acid (+);
capric acid (decanoic acid);
caproic acid (hexanoic acid);
caprylic acid (octanoic acid);
carbonic acid;
cinnamic acid;
citric acid;
cyclamic acid;
dodecylsulfuric acid;
ethane-1,2-disulfonic acid;
ethanesulfonic acid;
formic acid;
fumaric acid;
galactaric acid;
gentisic acid;
glucoheptonic acid (D);
gluconic acid (D);
glucuronic acid (D);
glutamic acid;
glutaric acid;
glycerophosphoric acid;
glycolic acid;
hippuric acid;
hydrobromic acid;
hydrochloric acid;
isobutyric acid;
lactic acid (DL);
lactobionic acid;
lauric acid;
maleic acid;
malic acid (−L);
malonic acid;
mandelic acid (DL);
methanesulfonic acid;
naphthalene-1,5-disulfonic acid;
naphthalene-2-sulfonic acid;
nicotinic acid;
nitric acid;
oleic acid;
oxalic acid;
palmitic acid;
pamoic acid;
phosphoric acid;
proprionic acid;
pyroglutamic acid (−L);
salicylic acid;
sebacic acid;
stearic acid;
succinic acid;
sulfuric acid;
tartaric acid (+L);
thiocyanic acid;
toluenesulfonic acid (p);
undecylenic acid

In some embodiments, the compound is a salt of acetic acid and any one of the compounds described in Table 3.

In some embodiments, the compound is a salt of adipic acid and any one of the compounds described in Table 3.

In some embodiments, the compound is a salt of ascorbic acid (L) and any one of the compounds described in Table 3.

In some embodiments, the compound is a salt of hydrobromic acid and any one of the compounds described in Table 3.

In some embodiments, the compound is a salt of hydrochloric acid and any one of the compounds described in Table 3.

In some embodiments, the compound is a salt of citric acid and any one of the compounds described in Table 3.

In some embodiments, the compound is a salt of glutamic acid and any one of the compounds described in Table 3.

In some embodiments, the compound is a salt of oxalic acid and any one of the compounds described in Table 3.

In some embodiments, the compound is a salt of formic acid and any one of the compounds described in Table 3.

In some embodiments, the compound is a salt of sulfuric acid and any one of the compounds described in Table 3.

In some aspects, the present disclosure provides a compound being an isotopic derivative (e.g., isotopically labeled compound) of any one of the compounds of the Formulae disclosed herein.

In some embodiments, the compound is an isotopic derivative of any one of the compounds described in Table 3 and prodrugs and pharmaceutically acceptable salts thereof.

In some embodiments, the compound is an isotopic derivative of any one of the compounds described in Table 3 and pharmaceutically acceptable salts thereof.

In some embodiments, the compound is an isotopic derivative of any one of prodrugs of the compounds described in Table 3 and pharmaceutically acceptable salts thereof.

In some embodiments, the compound is an isotopic derivative of any one of the compounds described in Table 3.

It is understood that the isotopic derivative can be prepared using any of a variety of art-recognized techniques. For example, the isotopic derivative can generally be prepared by carrying out the procedures disclosed in the Schemes and/or in the Examples described herein, by substituting an isotopically labeled reagent for a non-isotopically labeled reagent.

In some embodiments, the isotopic derivative is a deuterium labeled compound.

In some embodiments, the isotopic derivative is a deuterium labeled compound of any one of the compounds of the Formulae disclosed herein.

The term “isotopic derivative”, as used herein, refers to a derivative of a compound in which one or more atoms are isotopically enriched or labelled. For example, an isotopic derivative of a compound of Formula (A) is isotopically enriched with regard to, or labelled with, one or more isotopes as compared to the corresponding compound of Formula (A). In some embodiments, the isotopic derivative is enriched with regard to, or labelled with, one or more atoms selected from ²H, ¹³C, ¹⁴C, ¹⁵N, ¹⁸O, ²⁹SI, ³¹P, and ³⁴S. In some embodiments, the isotopic derivative is a deuterium labeled compound (i.e., being enriched with ²H with regard to one or more atoms thereof).

In some embodiments, the compound is a deuterium labeled compound of any one of the compounds described in Table 3 and prodrugs and pharmaceutically acceptable salts thereof.

In some embodiments, the compound is a deuterium labeled compound of any one of the compounds described in Table 3 and pharmaceutically acceptable salts thereof.

In some embodiments, the compound is a deuterium labeled compound of any one of the prodrugs of the compounds described in Table 3 and pharmaceutically acceptable salts thereof.

In some embodiments, the compound is a deuterium labeled compound of any one of the compounds described in Table 3.

It is understood that the deuterium labeled compound comprises a deuterium atom having an abundance of deuterium that is substantially greater than the natural abundance of deuterium, which is 0.015%.

In some embodiments, the deuterium labeled compound has a deuterium enrichment factor for each deuterium atom of at least 3500 (52.5% deuterium incorporation at each deuterium atom), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation). As used herein, the term “deuterium enrichment factor” means the ratio between the deuterium abundance and the natural abundance of a deuterium.

It is understood that the deuterium labeled compound can be prepared using any of a variety of art-recognized techniques. For example, the deuterium labeled compound can generally be prepared by carrying out the procedures disclosed in the Schemes and/or in the Examples described herein, by substituting a deuterium labeled reagent for a non-deuterium labeled reagent.

A compound of the disclosure or a pharmaceutically acceptable salt or solvate thereof that contains the aforementioned deuterium atom(s) is within the scope of the disclosure. Further, substitution with deuterium (i.e., ²H) may afford certain therapeutic advantages resulting from greater metabolic stability, e.g., increased in vivo half-life or reduced dosage requirements.

In some embodiments, the compound is a ¹⁸F labeled compound.

In some embodiments, the compound is a ¹²³1 labeled compound, a ¹²⁴1 labeled compound, a ¹²⁵1 labeled compound, a ¹²⁹1 labeled compound, a ¹³¹1 labeled compound, a 135I labeled compound, or any combination thereof.

In some embodiments, the compound is a ³³S labeled compound, a ³⁴S labeled compound, a ³⁵S labeled compound, a ³⁶S labeled compound, or any combination thereof.

It is understood that the ¹⁸F, 1231, 1241, 1251, 1291, 1311, 1351, 3, 34S 35S, and/or 36S labeled compound, can be prepared using any of a variety of art-recognized techniques. For example, the deuterium labeled compound can generally be prepared by carrying out the procedures disclosed in the Schemes and/or in the Examples described herein, by substituting a F, ¹²³I¹²⁴I¹²⁵I¹²⁹1 ¹³¹1, ¹³⁵1 ³S³⁴S ³⁵S and/or ³⁶S labeled reagent for anon-isotope labeled reagent.

A compound of the disclosure or a pharmaceutically acceptable salt or solvate thereof that contains one or more of the aforementioned F, ¹²³I¹²⁴1, ¹²⁵1, ¹²⁹1, ¹³¹1, ¹³⁵1³, 34S 35S, and ³⁶S atom(s) is within the scope of the disclosure. Further, substitution with isotope (e.g, ¹⁸F, ¹²³I¹²⁴1 ¹²⁵I¹²⁹I¹³¹I¹³⁵1³, ³⁴, ³⁵S, and/or ³⁶S) may afford certain therapeutic advantages resulting from greater metabolic stability, e.g., increased in vivo half-life or reduced dosage requirements.

For the avoidance of doubt, it is to be understood that, where in this specification a group is qualified by “described herein”, the said group encompasses the first occurring and broadest definition as well as each and all of the particular definitions for that group.

The various functional groups and substituents making up the compounds of the Formula (A) are typically chosen such that the molecular weight of the compound does not exceed 1000 Daltons. More usually, the molecular weight of the compound will be less than 900, for example less than 800, or less than 750, or less than 700, or less than 650 Daltons. More conveniently, the molecular weight is less than 600 and, for example, is 550 Daltons or less.

A suitable pharmaceutically acceptable salt of a compound of the disclosure is, for example, an acid-addition salt of a compound of the disclosure, which is sufficiently basic, for example, an acid-addition salt with, for example, an inorganic or organic acid, for example hydrochloric, hydrobromic, sulfuric, phosphoric, trifluoroacetic, formic, citric methane sulfonate or maleic acid. In addition, a suitable pharmaceutically acceptable salt of a compound of the disclosure which is sufficiently acidic is an alkali metal salt, for example a sodium or potassium salt, an alkaline earth metal salt, for example a calcium or magnesium salt, an ammonium salt or a salt with an organic base which affords a pharmaceutically acceptable cation, for example a salt with methylamine, dimethylamine, diethylamine, trimethylamine, piperidine, morpholine or tris-(2-hydroxyethyl)amine.

It will be understood that the compounds of any one of the Formulae disclosed herein and any pharmaceutically acceptable salts thereof, comprise stereoisomers, mixtures of stereoisomers, polymorphs of all isomeric forms of said compounds.

As used herein, the term “isomerism” means compounds that have identical molecular formulae but differ in the sequence of bonding of their atoms or in the arrangement of their atoms in space. Isomers that differ in the arrangement of their atoms in space are termed “stereoisomers.” Stereoisomers that are not mirror images of one another are termed “diastereoisomers,” and stereoisomers that are non-superimposable mirror images of each other are termed “enantiomers” or sometimes optical isomers. A mixture containing equal amounts of individual enantiomeric forms of opposite chirality is termed a “racemic mixture.”

As used herein, the term “chiral center” refers to a carbon atom bonded to four nonidentical substituents.

As used herein, the term “chiral isomer” means a compound with at least one chiral center.

Compounds with more than one chiral center may exist either as an individual diastereomer or as a mixture of diastereomers, termed “diastereomeric mixture.” When one chiral center is present, a stereoisomer may be characterized by the absolute configuration (R or S) of that chiral center.

Absolute configuration refers to the arrangement in space of the substituents attached to the chiral center. The substituents attached to the chiral center under consideration are ranked in accordance with the Sequence Rule of Cahn, Ingold and Prelog. (Cahn et al., Angew. Chem. Inter. Edit. 1966, 5, 385; errata 511; Cahn et al., Angew. Chem. 1966, 78, 413; Cahn and Ingold, J. Chem. Soc. 1951 (London), 612; Cahn et al., Experientia 1956, 12, 81; Cahn, J. Chem. Educ. 1964, 41, 116).

As used herein, the term “geometric isomer” means the diastereomers that owe their existence to hindered rotation about double bonds or a cycloalkyl linker (e.g., 1,3-cyclobutyl). These configurations are differentiated in their names by the prefixes cis and trans, or Z and E, which indicate that the groups are on the same or opposite side of the double bond in the molecule according to the Cahn-Ingold-Prelog rules.

It is to be understood that the compounds of the present disclosure may be depicted as different chiral isomers or geometric isomers. It is also to be understood that when compounds have chiral isomeric or geometric isomeric forms, all isomeric forms are intended to be included in the scope of the present disclosure, and the naming of the compounds does not exclude any isomeric forms, it being understood that not all isomers may have the same level of activity.

It is to be understood that the structures and other compounds discussed in this disclosure include all atropic isomers thereof. It is also to be understood that not all atropic isomers may have the same level of activity.

As used herein, the term “atropic isomers” are a type of stereoisomer in which the atoms of two isomers are arranged differently in space. Atropic isomers owe their existence to a restricted rotation caused by hindrance of rotation of large groups about a central bond. Such atropic isomers typically exist as a mixture, however as a result of recent advances in chromatography techniques, it has been possible to separate mixtures of two atropic isomers in select cases.

As used herein, the term “tautomer” is one of two or more structural isomers that exist in equilibrium and is readily converted from one isomeric form to another. This conversion results in the formal migration of a hydrogen atom accompanied by a switch of adjacent conjugated double bonds. Tautomers exist as a mixture of a tautomeric set in solution. In solutions where tautomerisation is possible, a chemical equilibrium of the tautomers will be reached. The exact ratio of the tautomers depends on several factors, including temperature, solvent, and pH. The concept of tautomers that are interconvertible by tautomerisations is called tautomerism. Of the various types of tautomerism that are possible, two are commonly observed. In keto-enol tautomerism a simultaneous shift of electrons and a hydrogen atom occurs. Ring-chain tautomerism arises as a result of the aldehyde group (—CHO) in a sugar chain molecule reacting with one of the hydroxy groups (—OH) in the same molecule to give it a cyclic (ring-shaped) form as exhibited by glucose.

It is to be understood that the compounds of the present disclosure may be depicted as different tautomers. It should also be understood that when compounds have tautomeric forms, all tautomeric forms are intended to be included in the scope of the present disclosure, and the naming of the compounds does not exclude any tautomer form. It will be understood that certain tautomers may have a higher level of activity than others.

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric centre, for example, it is bonded to four different groups, a pair of enantiomers is possible. An enantiomer can be characterised by the absolute configuration of its asymmetric centre and is described by the R- and S-sequencing rules of Cahn and Prelog, or by the manner in which the molecule rotates the plane of polarised light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The compounds of this disclosure may possess one or more asymmetric centres; such compounds can therefore be produced as individual (R)- or (S)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see discussion in Chapter 4 of “Advanced Organic Chemistry”, 4th edition J. March, John Wiley and Sons, New York, 2001), for example by synthesis from optically active starting materials or by resolution of a racemic form. Some of the compounds of the disclosure may have geometric isomeric centres (E- and Z—isomers). It is to be understood that the present disclosure encompasses all optical, diastereoisomers and geometric isomers and mixtures thereof that possess inflammasome inhibitory activity.

The present disclosure also encompasses compounds of the disclosure as defined herein which comprise one or more isotopic substitutions.

It is to be understood that the compounds of any Formula described herein include the compounds themselves, as well as their salts, and their solvates, if applicable. A salt, for example, can be formed between an anion and a positively charged group (e.g., amino) on a substituted compound disclosed herein. Suitable anions include chloride, bromide, iodide, sulfate, bisulfate, sulfamate, nitrate, phosphate, citrate, methanesulfonate, trifluoroacetate, glutamate, glucuronate, glutarate, malate, maleate, succinate, fumarate, tartrate, tosylate, salicylate, lactate, naphthalenesulfonate, and acetate (e.g., trifluoroacetate).

As used herein, the term “pharmaceutically acceptable anion” refers to an anion suitable for forming a pharmaceutically acceptable salt. Likewise, a salt can also be formed between a cation and a negatively charged group (e.g., carboxylate) on a substituted compound disclosed herein. Suitable cations include sodium ion, potassium ion, magnesium ion, calcium ion, and an ammonium cation such as tetramethylammonium ion or diethylamine ion. The substituted compounds disclosed herein also include those salts containing quaternary nitrogen atoms.

It is to be understood that the compounds of the present disclosure, for example, the salts of the compounds, can exist in either hydrated or unhydrated (the anhydrous) form or as solvates with other solvent molecules. Nonlimiting examples of hydrates include monohydrates, dihydrates, etc. Nonlimiting examples of solvates include ethanol solvates, acetone solvates, etc.

As used herein, the term “solvate” means solvent addition forms that contain either stoichiometric or non-stoichiometric amounts of solvent. Some compounds have a tendency to trap a fixed molar ratio of solvent molecules in the crystalline solid state, thus forming a solvate. If the solvent is water the solvate formed is a hydrate; and if the solvent is alcohol, the solvate formed is an alcoholate. Hydrates are formed by the combination of one or more molecules of water with one molecule of the substance in which the water retains its molecular state as H₂O.

As used herein, the term “analog” refers to a chemical compound that is structurally similar to another but differs slightly in composition (as in the replacement of one atom by an atom of a different element or in the presence of a particular functional group, or the replacement of one functional group by another functional group). Thus, an analog is a compound that is similar or comparable in function and appearance, but not in structure or origin to the reference compound.

As used herein, the term “derivative” refers to compounds that have a common core structure and are substituted with various groups as described herein.

As used herein, the term “bioisostere” refers to a compound resulting from the exchange of an atom or of a group of atoms with another, broadly similar, atom or group of atoms. The objective of a bioisosteric replacement is to create a new compound with similar biological properties to the parent compound. The bioisosteric replacement may be physicochemically or topologically based. Examples of carboxylic acid bioisosteres include, but are not limited to, acyl sulfonamides, tetrazoles, sulfonates and phosphonates. See, e.g., Patani and LaVoie, Chem. Rev. 96, 3147-3176, 1996.

It is also to be understood that certain compounds of any one of the Formulae disclosed herein may exist in solvated as well as unsolvated forms such as, for example, hydrated forms. A suitable pharmaceutically acceptable solvate is, for example, a hydrate such as hemi-hydrate, a mono-hydrate, a di-hydrate or a tri-hydrate. It is to be understood that the disclosure encompasses all such solvated forms that possess inflammasome inhibitory activity.

It is also to be understood that certain compounds of any one of the Formulae disclosed herein may exhibit polymorphism, and that the disclosure encompasses all such forms, or mixtures thereof, which possess inflammasome inhibitory activity. It is generally known that crystalline materials may be analysed using conventional techniques such as X-Ray Powder Diffraction analysis, Differential Scanning Calorimetry, Thermal Gravimetric Analysis, Diffuse Reflectance Infrared Fourier Transform (DRIFT) spectroscopy, Near Infrared (NIR) spectroscopy, solution and/or solid state nuclear magnetic resonance spectroscopy. The water content of such crystalline materials may be determined by Karl Fischer analysis.

Compounds of any one of the Formulae disclosed herein may exist in a number of different tautomeric forms and references to compound of Formula (A) include all such forms. For the avoidance of doubt, where a compound can exist in one of several tautomeric forms, and only one is specifically described or shown, all others are nevertheless embraced by Formula (A). Examples of tautomeric forms include keto-, enol-, and enolate-forms, as in, for example, the following tautomeric pairs: keto/enol (illustrated below), imine/enamine, amide/imino alcohol, amidine/amidine, nitroso/oxime, thioketone/enethiol, and nitro/aci-nitro.

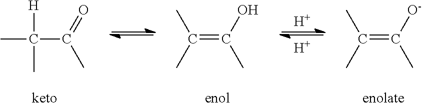

Compounds of any one of the Formulae disclosed herein containing an amine function may also form N-oxides. A reference herein to a compound of Formula (A) that contains an amine function also includes the N-oxide. Where a compound contains several amine functions, one or more than one nitrogen atom may be oxidized to form an N-oxide. Particular examples ofN-oxides are the N-oxides of a tertiary amine or a nitrogen atom of a nitrogen-containing heterocycle. N-oxides can be formed by treatment of the corresponding amine with an oxidizing agent such as hydrogen peroxide or a peracid (e.g. a peroxycarboxylic acid), see for example Advanced Organic Chemistry, by Jerry March, 4th Edition, Wiley Interscience, pages. More particularly, N-oxides can be made by the procedure of L. W. Deady (Syn. Comm. 1977, 7, 509-514) in which the amine compound is reacted with meta-chloroperoxybenzoic acid (mCPBA), for example, in an inert solvent such as dichloromethane.

The compounds of any one of the Formulae disclosed herein may be administered in the form of a prodrug which is broken down in the human or animal body to release a compound of the disclosure. A prodrug may be used to alter the physical properties and/or the pharmacokinetic properties of a compound of the disclosure. A prodrug can be formed when the compound of the disclosure contains a suitable group or substituent to which a property-modifying group can be attached. Examples of prodrugs include derivatives containing in vivo cleavable alkyl or acyl substituents at the ester or amide group in any one of the Formulae disclosed herein.

Accordingly, the present disclosure includes those compounds of any one of the Formulae disclosed herein as defined hereinbefore when made available by organic synthesis and when made available within the human or animal body by way of cleavage of a prodrug thereof. Accordingly, the present disclosure includes those compounds of any one of the Formulae disclosed herein that are produced by organic synthetic means and also such compounds that are produced in the human or animal body by way of metabolism of a precursor compound, that is a compound of any one of the Formulae disclosed herein may be a synthetically produced compound or a metabolically-produced compound.

A suitable pharmaceutically acceptable prodrug of a compound of any one of the Formulae disclosed herein is one that is based on reasonable medical judgment as being suitable for administration to the human or animal body without undesirable pharmacological activities and without undue toxicity. Various forms of prodrug have been described, for example in the following documents: a) Methods in Enzymology, Vol. 42, p. 309-396, edited by K. Widder, et al. (Academic Press, 1985); b) Design of Pro-drugs, edited by H. Bundgaard, (Elsevier, 1985); c) A Textbook of Drug Design and Development, edited by Krogsgaard-Larsen and H. Bundgaard, Chapter 5 “Design and Application of Pro-drugs”, by H. Bundgaard p. 113-191 (1991); d) H.

Bundgaard, Advanced Drug Delivery Reviews, 8, 1-38 (1992); e) H. Bundgaard, et al., Journal of Pharmaceutical Sciences, 77, 285 (1988); f) N. Kakeya, et al., Chem. Pharm. Bull., 32, 692 (1984); g) T. Higuchi and V. Stella, “Pro-Drugs as Novel Delivery Systems”, A.C.S. Symposium Series, Volume 14; and h) E. Roche (editor), “Bioreversible Carriers in Drug Design”, Pergamon Press, 1987.

A suitable pharmaceutically acceptable prodrug of a compound of any one of the Formulae disclosed herein that possesses a hydroxy group is, for example, an in vivo cleavable ester or ether thereof. An in vivo cleavable ester or ether of a compound of any one of the Formulae disclosed herein containing a hydroxy group is, for example, a pharmaceutically acceptable ester or ether which is cleaved in the human or animal body to produce the parent hydroxy compound. Suitable pharmaceutically acceptable ester forming groups for a hydroxy group include inorganic esters such as phosphate esters (including phosphoramidic cyclic esters). Further suitable pharmaceutically acceptable ester forming groups for a hydroxy group include C₁-C₁₀ alkanoyl groups such as acetyl, benzoyl, phenylacetyl and substituted benzoyl and phenylacetyl groups, C₁-C₁₀ alkoxycarbonyl groups such as ethoxycarbonyl, N,N—(C₁-C₆ alkyl)₂carbamoyl, 2-dialkylaminoacetyl and 2-carboxyacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁-C₄ alkyl)piperazin-1-ylmethyl. Suitable pharmaceutically acceptable ether forming groups for a hydroxy group include a-acyloxyalkyl groups such as acetoxymethyl and pivaloyloxymethyl groups.

A suitable pharmaceutically acceptable prodrug of a compound of any one of the Formulae disclosed herein that possesses a carboxy group is, for example, an in vivo cleavable amide thereof, for example an amide formed with an amine such as ammonia, a C₁₄alkylamine such as methylamine, a (C₁-C₄ alkyl)₂amine such as dimethylamine, N-ethyl-N-methylamine or diethylamine, a C₁-C₄ alkoxy-C₂-C₄ alkylamine such as 2-methoxyethylamine, a phenyl-C₁-C₄ alkylamine such as benzylamine and amino acids such as glycine or an ester thereof.

A suitable pharmaceutically acceptable prodrug of a compound of any one of the Formulae disclosed herein that possesses an amino group is, for example, an in vivo cleavable amide derivative thereof. Suitable pharmaceutically acceptable amides from an amino group include, for example an amide formed with C₁-C₁₀ alkanoyl groups such as an acetyl, benzoyl, phenylacetyl and substituted benzoyl and phenylacetyl groups. Examples of ring substituents on the phenylacetyl and benzoyl groups include aminomethyl, N-alkylaminomethyl, N,N-dialkylaminomethyl,morpholinomethyl,piperazin-1-ylmethyl and 4-(C₁-C₄ alkyl)piperazin-1-ylmethyl.

The in vivo effects of a compound of any one of the Formulae disclosed herein may be exerted in part by one or more metabolites that are formed within the human or animal body after administration of a compound of any one of the Formulae disclosed herein. As stated hereinbefore, the in vivo effects of a compound of any one of the Formulae disclosed herein may also be exerted by way of metabolism of a precursor compound (a prodrug).

### Method of Synthesizing the Compounds

The compounds of the present invention may be made by a variety of methods, including standard chemistry. Suitable synthetic routes are depicted in the Schemes given below.

The compounds of Formula (A) may be prepared by methods known in the art of organic synthesis as set forth in part by the following synthetic schemes. In the schemes described below, it is well understood that protecting groups for sensitive or reactive groups are employed where necessary in accordance with general principles or chemistry. Protecting groups are manipulated according to standard methods of organic synthesis (T. W. Greene and P. G. M. Wuts, “Protective Groups in Organic Synthesis”, Third edition, Wiley, New York 1999). These groups are removed at a convenient stage of the compound synthesis using methods that are readily apparent to those skilled in the art. The selection processes, as well as the reaction conditions and order of those skilled in the art will recognize if a stereocenter exists in the compounds of Formula (A). Accordingly, the present invention includes both possible stereoisomers (unless specified in the synthesis) and includes not only racemic compounds but the individual enantiomers and/or diastereomers as well. When a compound is desired as a single enantiomer or diastereomer, it may be obtained by stereospecific synthesis or by resolution of the final product or any convenient intermediate. Resolution of the final product, an intermediate, or a starting material may be affected by any suitable method known in the art. See, for example, “Stereochemistry of Organic Compounds” by E. L. Eliel, S. H. Wilen, and L. N. Mander (Wiley-lnterscience, 1994).

The compounds described herein may be made from commercially available starting materials or synthesized using known organic, inorganic, and/or enzymatic processes.

### Preparation of Compounds

The compounds of the present invention can be prepared in a number of ways well known to those skilled in the art of organic synthesis. By way of example, compounds of the present invention can be synthesized using the methods described below, together with synthetic methods known in the art of synthetic organic chemistry, or variations thereon as appreciated by those skilled in the art. Suitable methods include but are not limited to those methods described below. Compounds of the present invention can be synthesized by following the steps outlined in General Procedure which comprise different sequences of assembling intermediates or compounds. Starting materials are either commercially available or made by known procedures in the reported literature or as illustrated below.

### GENERAL PROCEDURE

In general, the compound of the Formula (A) can be prepared using any of reactions presented at the Scheme 1:

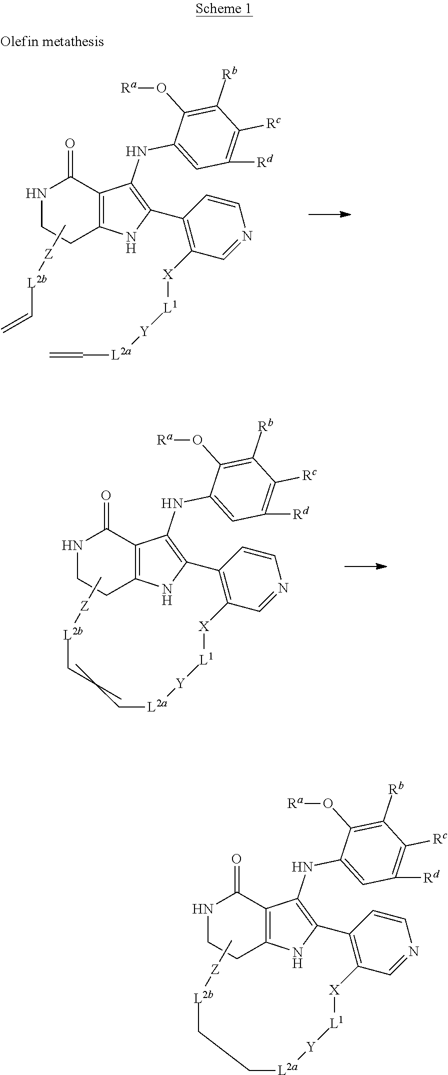

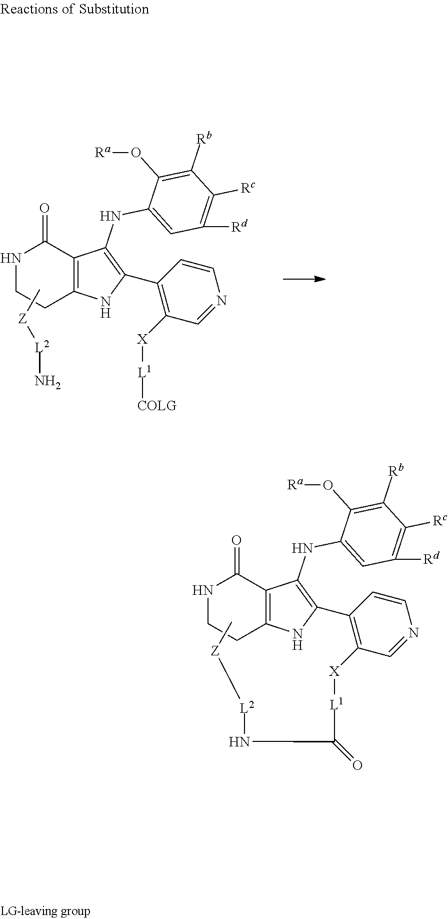

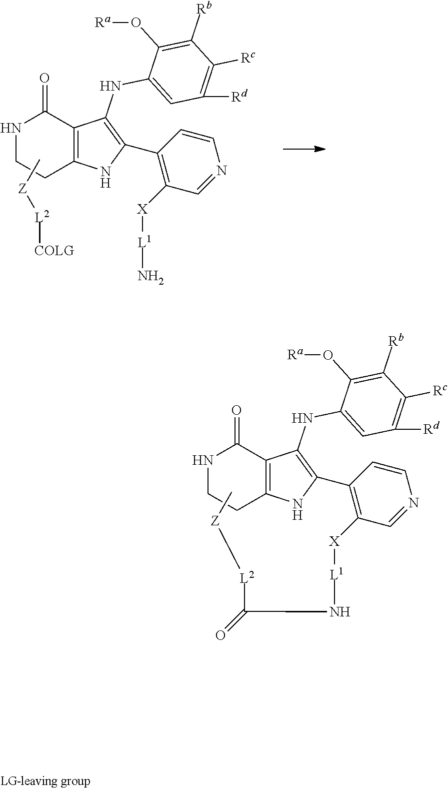

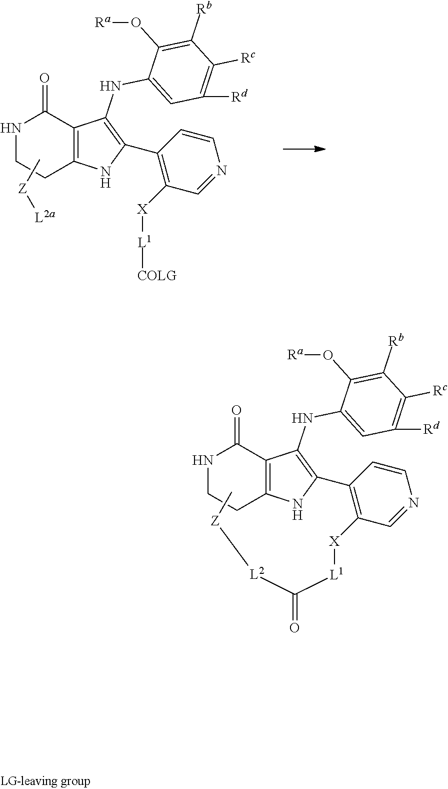

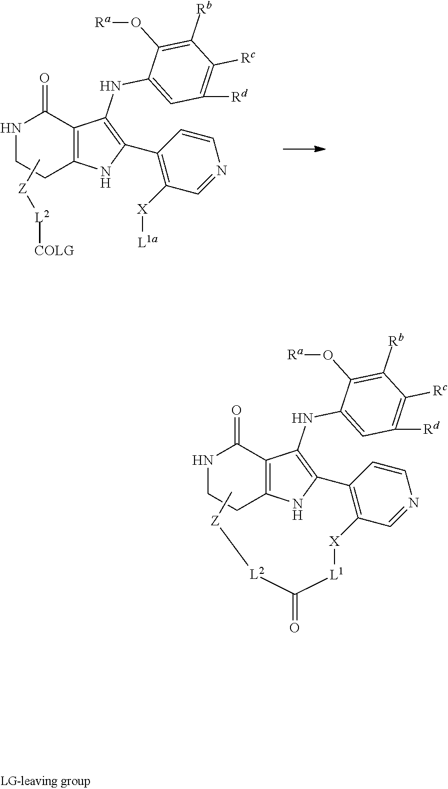

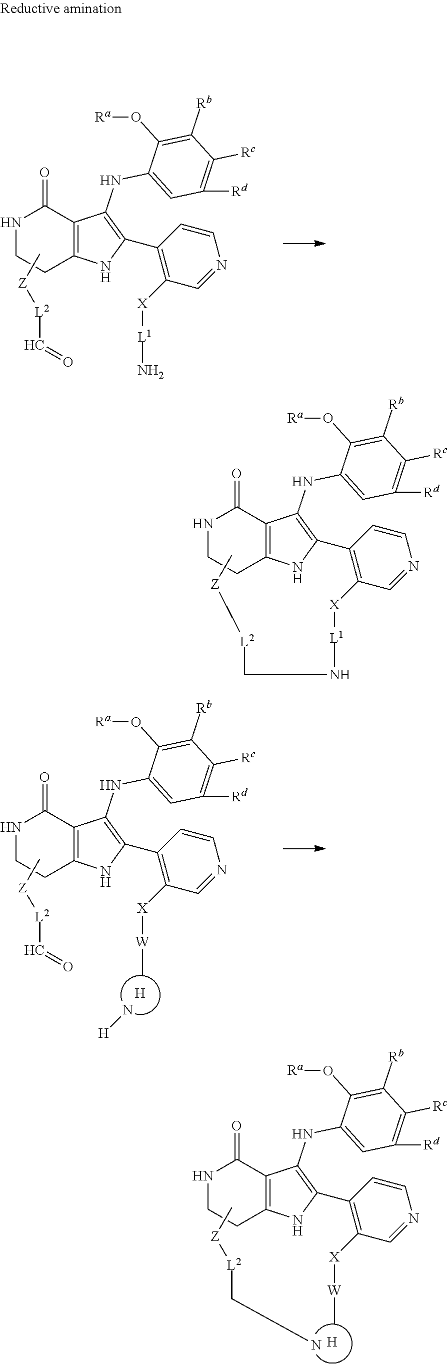

All reagents may be commercially available compounds itself or products of synthesis from commercially available reagents. For preparation these reagents may be used one step or multistep synthetic procedures, including but not limited procedures described herein in preparative part.

In general, the compound of the Formula (I) can be prepared using any of reactions presented at the Scheme 2:

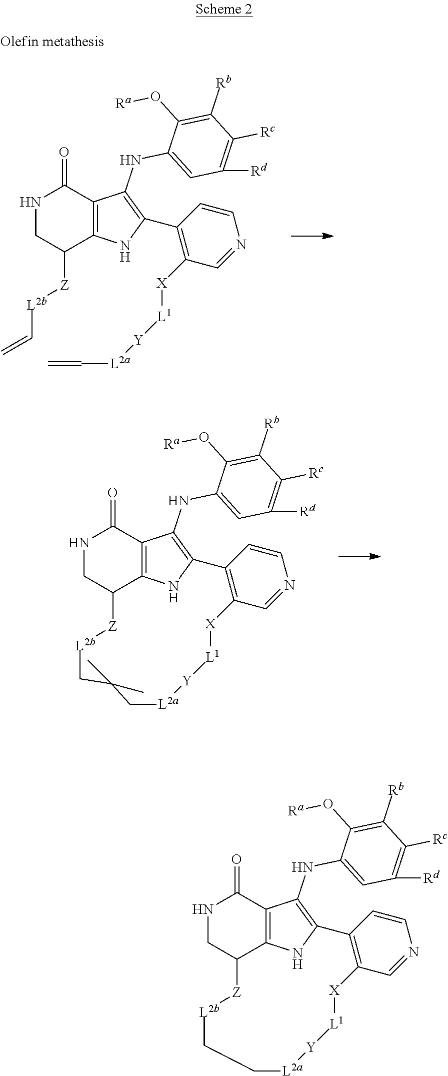

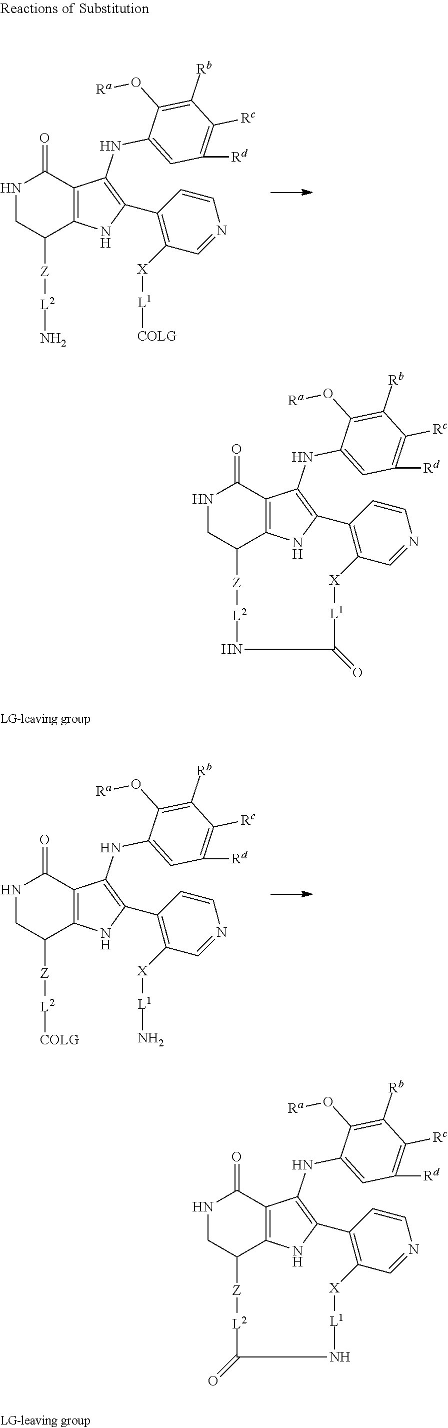

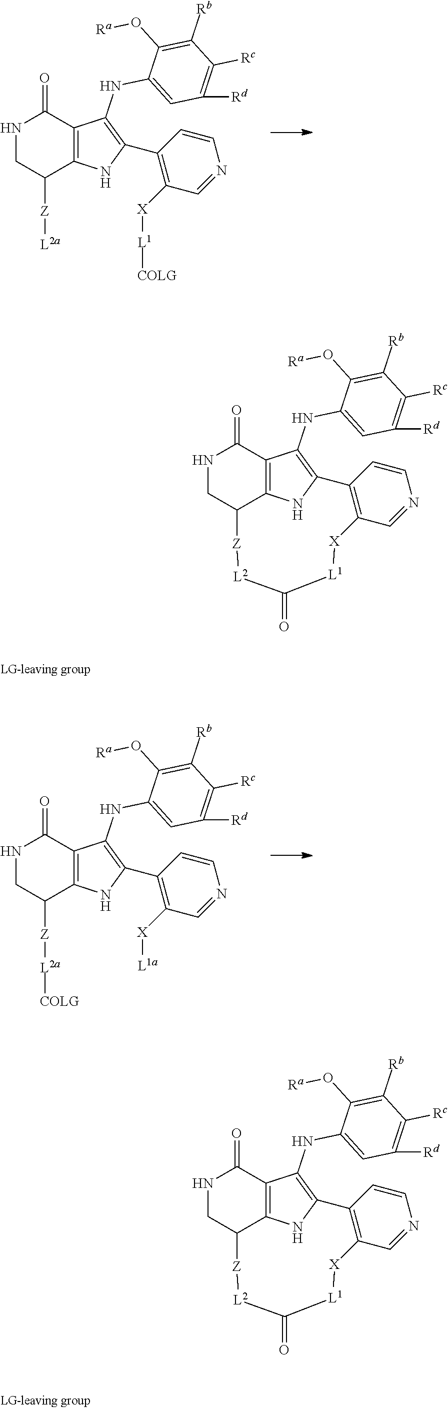

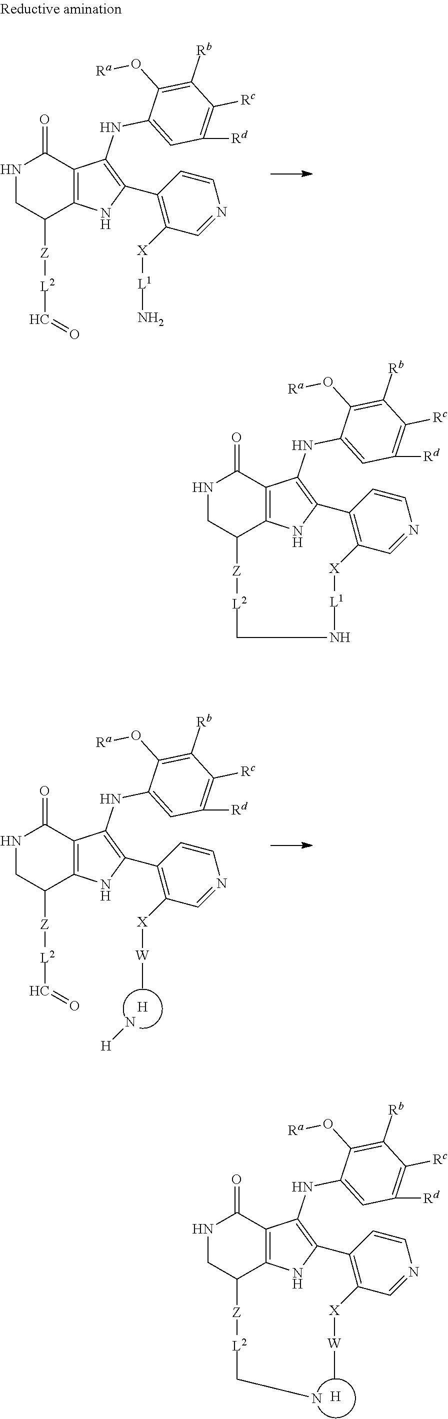

In general, the compound of the Formula (II) can be prepared using any of reactions presented at the Scheme 3:

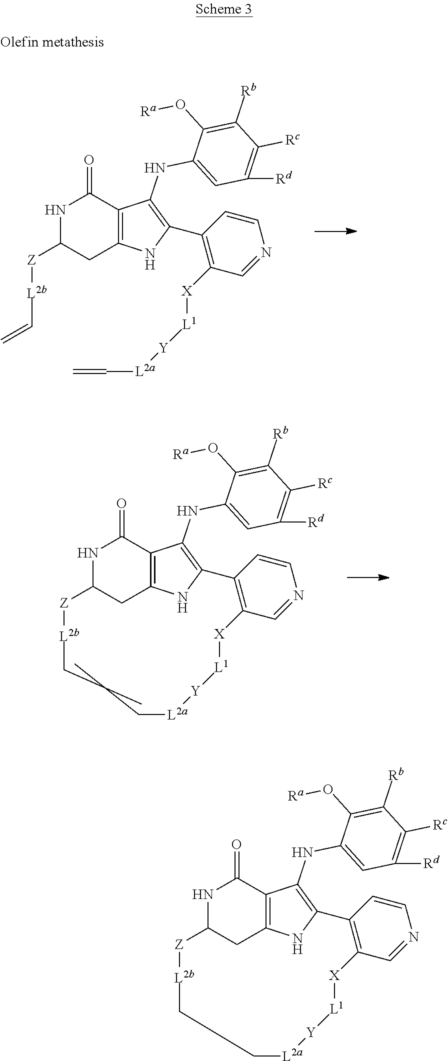

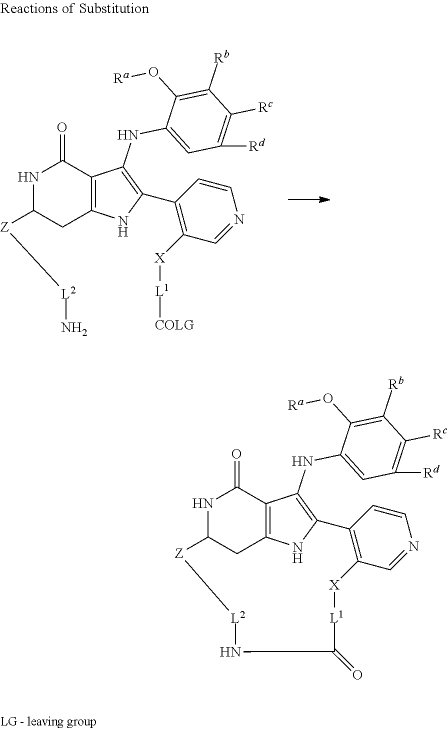

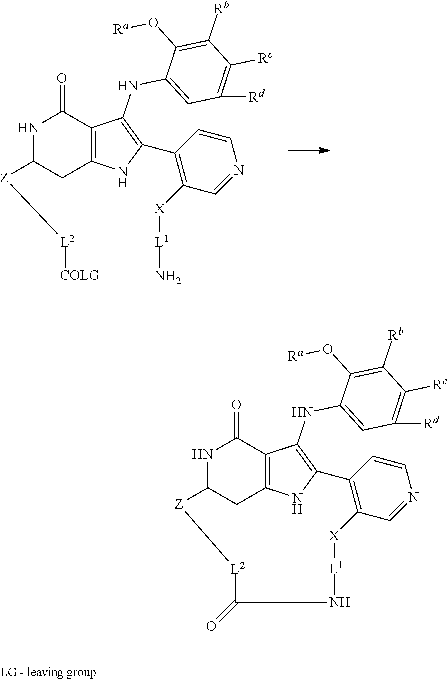

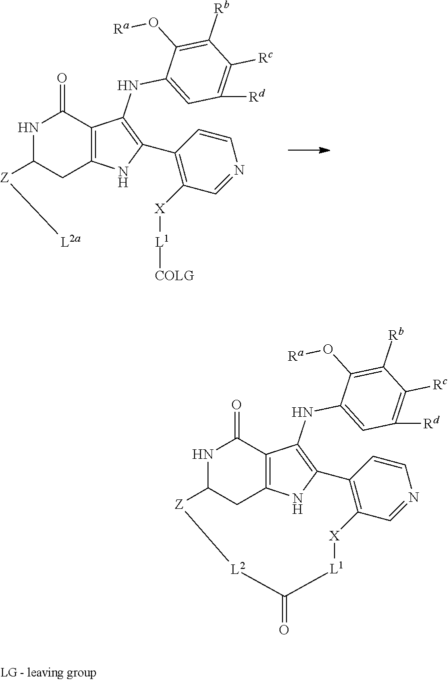

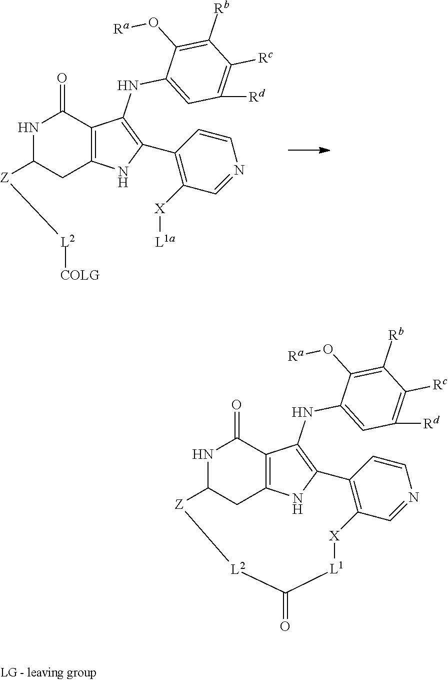

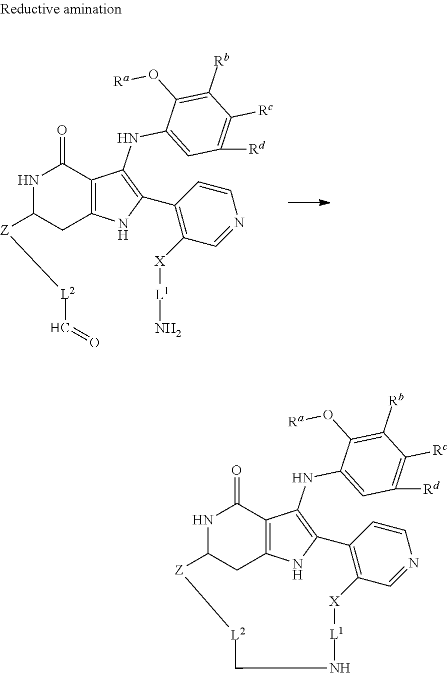

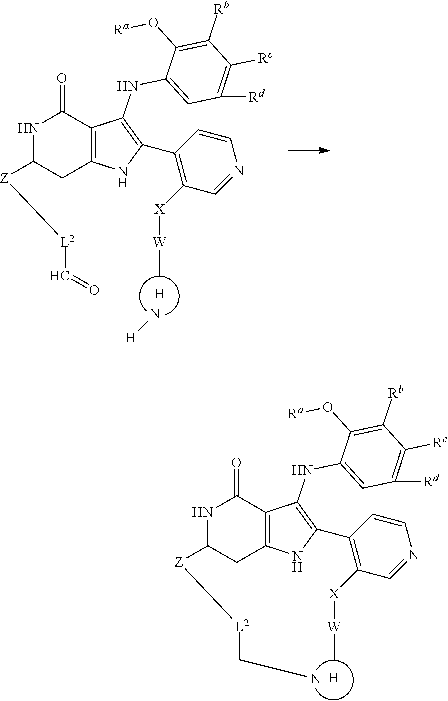

### General Procedure A.1

In more specific aspect, the compound of the Formula (I) can be prepared using sequences of reactions presented at the Scheme 4:

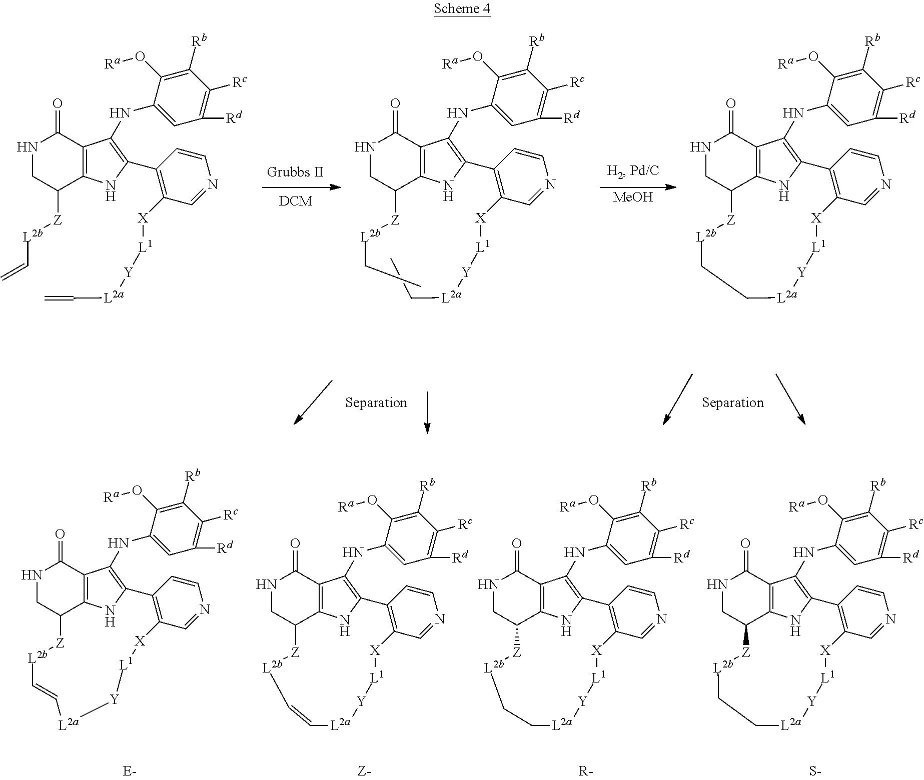

wherein, L^{2a} and L^{2b} are each independently parts of L² —group of atoms or a bond.

All reagents may be commercially available compounds itself or products of synthesis from commercially available reagents. For preparation these reagents may be used one step or multistep synthetic procedures, including but not limited procedures described herein in preparative part.

### General Procedure A.2

In another more specific aspect, the compound of the Formula (I) can be prepared using sequences of reactions presented at the Scheme 5:

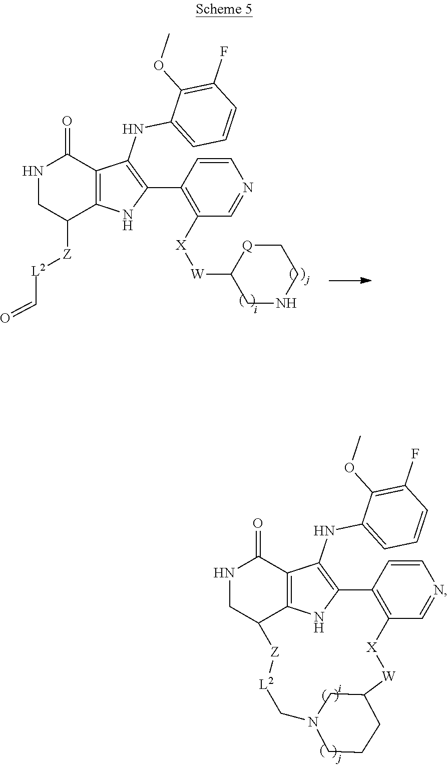

wherein i is an integer selected from 0, 1, 2; j is an integer selected from 0, 1, 2; Q is selected from CH₂, NH, O, S, S(O), S(O)₂ and all other variables are as defined herein.

### Specific Example of Preparation

In specific non-limiting approach, the compound of the Formula (I) can be prepared using sequences of reactions presented at the Scheme 6:

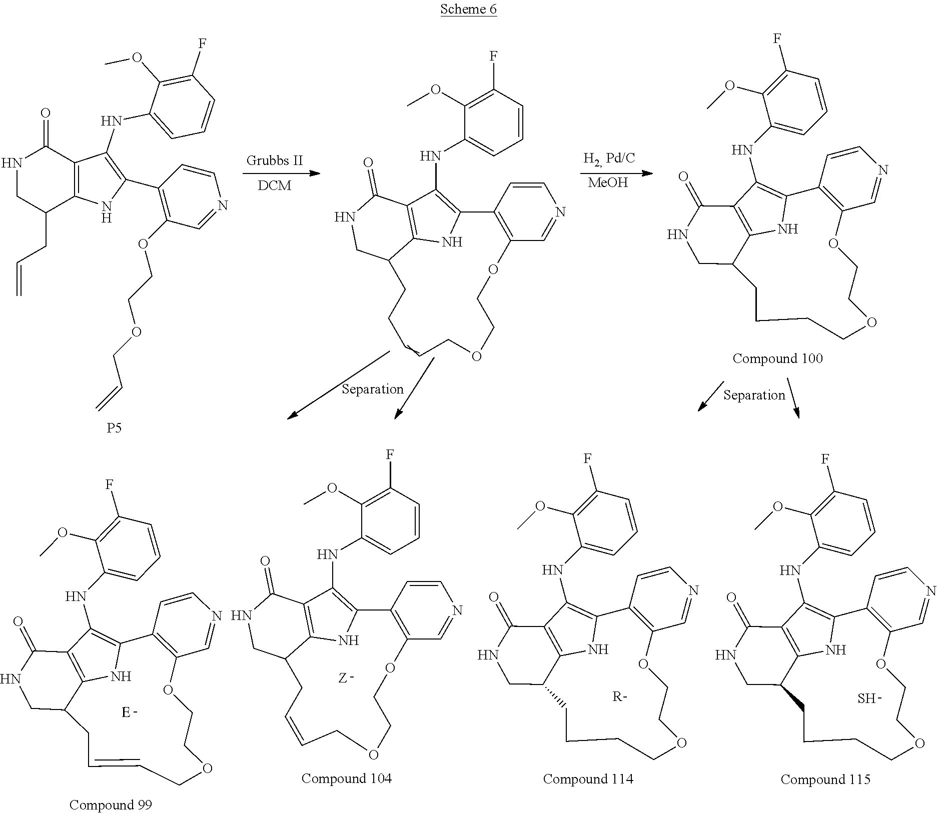

All reagents may be commercially available compounds itself or products of synthesis from commercially available reagents. For preparation these reagents may be used one step or multistep synthetic procedures, including but not limited procedures described herein in preparative part.

It should be obvious for specialist in this field that any of compound of Formula (A) obtained according to the procedures described above may be a subject for further transformation and modification that will be led to obtain other compound of Formula (A).

It should be obvious for specialist in this field that any of compound of Formula (I) obtained according to the procedures described above may be a subject for further transformation and modification that will be led to obtain other compound of Formula (I).

It should be obvious for specialist in this field that any of compound of Formula (II) obtained according to the procedures described above may be a subject for further transformation and modification that will be led to obtain other compound of Formula (II).

### Biological Assays

Compounds designed, selected and/or optimized by methods described above, once produced, can be characterized using a variety of assays known to those skilled in the art to determine whether the compounds have biological activity. For example, the molecules can be characterized by conventional assays, including but not limited to those assays described below, to determine whether they have a predicted activity, binding activity and/or binding specificity.

Furthermore, high-throughput screening can be used to speed up analysis using such assays. As a result, it can be possible to rapidly screen the molecules described herein for activity, using techniques known in the art. General methodologies for performing high-throughput screening are described, for example, in Devlin (1998) High Throughput Screening, Marcel Dekker; and U.S. Pat. No. 5,763,263. High-throughput assays can use one or more different assay techniques including, but not limited to, those described below.

Various in vitro or in vivo biological assays may be suitable for detecting the effect of the compounds of the present disclosure. These in vitro or in vivo biological assays can include, but are not limited to, enzymatic activity assays, electrophoretic mobility shift assays, reporter gene assays, in vitro cell viability assays, and the assays described herein.

### Pharmaceutical Compositions

In some aspects, the present disclosure provides a pharmaceutical composition comprising a compound of the present disclosure as an active ingredient. In some embodiments, the present disclosure provides a pharmaceutical composition comprising at least one compound of each of the formulae described herein, or a pharmaceutically acceptable salt or solvate thereof, and one or more pharmaceutically acceptable carriers or excipients. In some embodiments, the present disclosure provides a pharmaceutical composition comprising at least one compound selected from Table 3.

As used herein, the term “composition” is intended to encompass a product comprising the specified ingredients in the specified amounts, as well as any product which results, directly or indirectly, from combination of the specified ingredients in the specified amounts.

Solid form preparations include powders, tablets, pills, capsules, cachets, suppositories, and dispersible granules. A solid carrier may be one or more substances which may also act as diluents, flavoring agents, solubilizers, lubricants, suspending agents, binders, preservatives, tablet disintegrating agents, or an encapsulating material. In powders, the carrier generally is a finely divided solid which is a mixture with the finely divided active component. In tablets, the active component generally is mixed with the carrier having the necessary binding capacity in suitable proportions and compacted in the shape and size desired. Suitable carriers include but are not limited to magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. Solid form preparations may contain, in addition to the active component, colorants, flavors, stabilizers, buffers, artificial and natural sweeteners, dispersants, thickeners, solubilizing agents, and the like.

Liquid formulations also are suitable for oral administration include liquid formulation including emulsions, syrups, elixirs, aqueous solutions, aqueous suspensions. These include solid form preparations which are intended to be converted to liquid form preparations shortly before use. Emulsions may be prepared in solutions, for example, in aqueous propylene glycol solutions or may contain emulsifying agents such as lecithin, sorbitan monooleate, or acacia. Aqueous solutions can be prepared by dissolving the active component in water and adding suitable colorants, flavors, stabilizing, and thickening agents. Aqueous suspensions can be prepared by dispersing the finely divided active component in water with viscous material, such as natural or synthetic gums, resins, methylcellulose, sodium carboxymethylcellulose, and other well-known suspending agents.

The compounds of the present invention may be formulated for parenteral administration (e.g., by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, for example solutions in aqueous polyethylene glycol. Examples of oily or nonaqueous carriers, diluents, solvents or vehicles include propylene glycol, polyethylene glycol, vegetable oils (e.g., olive oil), and injectable organic esters (e.g., ethyl oleate), and may contain formulatory agents such as preserving, wetting, emulsifying or suspending, stabilizing and/or dispersing agents. Alternatively, the active ingredient may be in powder form, obtained by aseptic isolation of sterile solid or by lyophilization from solution for constitution before use with a suitable vehicle, e.g., sterile, pyrogen-free water.

The compounds of present disclosure can be formulated for oral administration in forms such as tablets, capsules (each of which includes sustained release or timed release formulations), pills, powders, granules, elixirs, tinctures, suspensions, syrups and emulsions. The compounds of present disclosure on can also be formulated for intravenous (bolus or in-fusion), intraperitoneal, topical, subcutaneous, intramuscular or transdermal (e.g., patch) administration, all using forms well known to those of ordinary skill in the pharmaceutical arts.

The formulation of the present disclosure may be in the form of an aqueous solution comprising an aqueous vehicle. The aqueous vehicle component may comprise water and at least one pharmaceutically acceptable excipient. Suitable acceptable excipients include those selected from the group consisting of a solubility enhancing agent, chelating agent, preservative, tonicity agent, viscosity/suspending agent, buffer, and pH modifying agent, and a mixture thereof.

Any suitable solubility enhancing agent can be used. Examples of a solubility enhancing agent include cyclodextrin, such as those selected from the group consisting of hydroxypropyl-p-cyclodextrin, methyl-o-cyclodextrin, randomly methylated-o-cyclodextrin, ethylated-o-cyclodextrin, triacetyl-o-cyclodextrin, peracetylated-o-cyclodextrin, carboxymethyl-p-cyclodextrin, hydroxyethyl-o-cyclodextrin, 2-hydroxy-3-(trimethylammonio)propyl-p-cyclodextrin, glucosyl-o-cyclodextrin, sulfated P-cyclodextrin (S—O—CD), maltosyl-o-cyclodextrin, P-cyclodextrin sulfobutyl ether, branched-O-cyclodextrin, hydroxypropyl-y-cyclodextrin, randomly methylated-y-cyclodextrin, and trimethyl-y-cyclodextrin, and mixtures thereof.

Any suitable chelating agent can be used. Examples of a suitable chelating agent include those selected from the group consisting of ethylenediaminetetraacetic acid and metal salts thereof, disodium edetate, trisodium edetate, and tetrasodium edetate, and mixtures thereof.

Any suitable preservative can be used. Examples of a preservative include those selected from the group consisting of quaternary ammonium salts such as benzalkonium halides (preferably benzalkonium chloride), chlorhexidine gluconate, benzethonium chloride, cetyl pyridinium chloride, benzyl bromide, phenylmercury nitrate, phenylmercury acetate, phenylmercury neodecanoate, merthiolate, methylparaben, propylparaben, sorbic acid, potassium sorbate, sodium benzoate, sodium propionate, ethyl p-hydroxybenzoate, propylaminopropyl biguanide, and butyl-p-hydroxybenzoate, and sorbic acid, and mixtures thereof.

In some embodiments, examples of a preservative include those selected from the group consisting of quaternary ammonium salts such as benzalkonium halides (preferably benzalkonium chloride), chlorhexidine gluconate, benzethonium chloride, cetyl pyridinium chloride, benzyl bromide, phenylmercury nitrate, merthiolate, methylparaben, propylparaben, sorbic acid, potassium sorbate, sodium benzoate, sodium propionate, ethyl p-hydroxybenzoate, propylaminopropyl biguanide, and butyl-p-hydroxybenzoate, and sorbic acid, and mixtures thereof.

The aqueous vehicle may also include a tonicity agent to adjust the tonicity (osmotic pressure). The tonicity agent can be selected from the group consisting of a glycol (such as propylene glycol, diethylene glycol, triethylene glycol), glycerol, dextrose, glycerin, mannitol, potassium chloride, and sodium chloride, and a mixture thereof. In some embodiments, the tonicity agent is selected from the group consisting of a glycol (such as propylene glycol, triethylene glycol), glycerol, dextrose, glycerin, mannitol, potassium chloride, and sodium chloride, and a mixture thereof.

The aqueous vehicle may also contain a viscosity/suspending agent. Suitable viscosity/suspending agents include those selected from the group consisting of cellulose derivatives, such as methyl cellulose, ethyl cellulose, hydroxyethylcellulose, polyethylene glycols (such as polyethylene glycol 300, polyethylene glycol 400), carboxymethyl cellulose, hydroxypropylmethyl cellulose, and cross-linked acrylic acid polymers (carbomers), such as polymers of acrylic acid cross-linked with polyalkenyl ethers or divinyl glycol (Carbopols—such as Carbopol 934, Carbopol 934P, Carbopol 971, Carbopol 974 and Carbopol 974P), and a mixture thereof.

In order to adjust the formulation to an acceptable pH (typically a pH range of about 5.0 to about 9.0, more preferably about 5.5 to about 8.5, particularly about 6.0 to about 8.5, about 7.0 to about 8.5, about 7.2 to about 7.7, about 7.1 to about 7.9, or about 7.5 to about 8.0), the formulation may contain a pH modifying agent. The pH modifying agent is typically a mineral acid or metal hydroxide base, selected from the group of potassium hydroxide, sodium hydroxide, and hydrochloric acid, and mixtures thereof, and preferably sodium hydroxide and/or hydrochloric acid. These acidic and/or basic pH modifying agents are added to adjust the formulation to the target acceptable pH range. Hence it may not be necessary to use both acid and base—depending on the formulation, the addition of one of the acid or base may be sufficient to bring the mixture to the desired pH range.

The aqueous vehicle may also contain a buffering agent to stabilize the pH. When used, the buffer is selected from the group consisting of a phosphate buffer (such as sodium dihydrogen phosphate and disodium hydrogen phosphate), a borate buffer (such as boric acid, or salts thereof including disodium tetraborate), a citrate buffer (such as citric acid, or salts thereof including sodium citrate), and E-aminocaproic acid, and mixtures thereof.

The formulation may further comprise a wetting agent. Suitable classes of wetting agents include those selected from the group consisting of polyoxypropylene-polyoxyethylene block copolymers (poloxamers), polyethoxylated ethers of castor oils, polyoxyethylenated sorbitan esters (polysorbates), polymers of oxyethylated octyl phenol (Tyloxapol), polyoxyl 40 stearate, fatty acid glycol esters, fatty acid glyceryl esters, sucrose fatty esters, and polyoxyethylene fatty esters, and mixtures thereof.

Oral compositions generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

According to a further aspect of the disclosure there is provided a pharmaceutical composition which comprises a compound of the disclosure as defined hereinbefore, or a pharmaceutically acceptable salt, hydrate or solvate thereof, in association with a pharmaceutically acceptable diluent or carrier.

In some embodiments, a pharmaceutical composition described herein may further comprise one or more additional pharmaceutically active agents.

The compositions of the disclosure may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular, intraperitoneal or intramuscular dosing or as a suppository for rectal dosing).

The compositions of the disclosure may be obtained by conventional procedures using conventional pharmaceutical excipients, well known in the art. Thus, compositions intended for oral use may contain, for example, one or more coloring, sweetening, flavoring and/or preservative agents.

A therapeutically effective amount of a compound of the present disclosure for use in therapy is an amount sufficient to treat or prevent a EGFR related condition referred to herein, slow its progression and/or reduce the symptoms associated with the condition.

A therapeutically effective amount of a compound of the present disclosure for use in therapy is an amount sufficient to treat an EGFR related condition referred to herein, slow its progression and/or reduce the symptoms associated with the condition.

The size of the dose for therapeutic or prophylactic purposes of a compound of Formula (A) will naturally vary according to the nature and severity of the conditions, the age and sex of the animal or subject and the route of administration, according to well-known principles of medicine.

### Methods of Use

In some aspects, the present disclosure provides a method of inhibiting of EGFR (e.g., in vitro or in vivo), comprising contacting a cell with a therapeutically effective amount of a compound of the present disclosure or a pharmaceutically acceptable salt thereof.

In some aspects, the present disclosure provides a method of treating or preventing a disease or disorder disclosed herein in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating a disease or disorder disclosed herein in a subject in need thereof, comprising administering to the subject a therapeutically effective amount of a compound of the present disclosure or a pharmaceutically acceptable salt thereof, or a pharmaceutical composition of the present disclosure.

In some embodiments, the disease or disorder is associated with EGFR. In some embodiments, the disease or disorder is a disease or disorder in which EGFR is implicated.

The compounds of the invention are also useful in treating diseases associated with EGFR. For example, diseases and conditions treatable according to the methods of the invention include Lung Cancer; Lung Cancer Susceptibility 3 (LNCR3); Lung Squamous Cell Carcinoma; Gliosarcoma; Brain Stem Glioma; Adenocarcinoma; Colorectal Cancer; Glioblastoma; Breast Cancer; Squamous Cell Carcinoma; Small Cell Cancer of the Lung; Lung Squamous Cell Carcinoma; Inflammatory Skin and Bowel Disease, Neonatal, 2; Gastric Cancer; Prostate Cancer; Squamous Cell Carcinoma, Head and Neck; Pancreatic Cancer; Brain Cancer; Ovarian Cancer; Esophageal Cancer; Giant Cell Glioblastoma; Gliosarcoma; Lung Benign Neoplasm; Glioma; Exanthem; Endometrial Cancer; Adenoid Cystic Carcinoma; Bladder Cancer; Cowden Syndrome 1; Bronchiolo-Alveolar Adenocarcinoma; Oligodendroglioma; Hepatocellular Carcinoma; Lung Non-Squamous Non-Small Cell Carcinoma; High Grade Glioma; Glial Tumor; Laryngeal Squamous Cell Carcinoma; Renal Cell Carcinoma, Nonpapillary; Chronic Kidney Disease; Nasopharyngeal Carcinoma; Oral Squamous Cell Carcinoma; Lung Disease; Interstitial Lung Disease; Adenosquamous Lung Carcinoma; Bile Duct Cancer; Meningioma, Familial; Small Cell Carcinoma; Tumor Predisposition Syndrome; Pancreatic Adenocarcinoma; Diarrhea; Breast Ductal Carcinoma.

In some embodiments, the disease or disorder is a Lung Cancer.

In some aspects, the present disclosure provides a method of treating or preventing a Lung Cancer in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating a Lung Cancer in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a compound of the present disclosure or a pharmaceutically acceptable salt thereof for use in inhibiting of EGFR (e.g., in vitro or in vivo).

In some aspects, the present disclosure provides a compound of the present disclosure or a pharmaceutically acceptable salt thereof for use in treating or preventing a disease or disorder disclosed herein.

In some aspects, the present disclosure provides a compound of the present disclosure or a pharmaceutically acceptable salt thereof for use in treating a disease or disorder disclosed herein.

In some aspects, the present disclosure provides a compound of the present disclosure or a pharmaceutically acceptable salt thereof for use in treating or preventing a Lung Cancer in a subject in need thereof.

In some aspects, the present disclosure provides a compound of the present disclosure or a pharmaceutically acceptable salt thereof for use in treating Lung Cancer in a subject in need thereof.

In some aspects, the present disclosure provides use of a compound of the present disclosure or a pharmaceutically acceptable salt thereof in the manufacture of a medicament for inhibiting of EGFR (e.g., in vitro or in vivo).

In some aspects, the present disclosure provides use of a compound of the present disclosure or a pharmaceutically acceptable salt thereof in the manufacture of a medicament for treating or preventing a disease or disorder disclosed herein.

In some aspects, the present disclosure provides use of a compound of the present disclosure or a pharmaceutically acceptable salt thereof in the manufacture of a medicament for treating a disease or disorder disclosed herein.

In some aspects, the present disclosure provides use of a compound of the present disclosure or a pharmaceutically acceptable salt thereof in the manufacture of a medicament for treating or preventing Lung Cancer in a subject in need thereof.

In some aspects, the present disclosure provides use of a compound of the present disclosure or a pharmaceutically acceptable salt thereof in the manufacture of a medicament for treating Lung Cancer in a subject in need thereof.

The present disclosure provides compounds that function as inhibitors of EGFR (e.g., in vitro or in vivo). The present disclosure therefore provides a method of inhibiting of EGFR in vitro or in vivo, said method comprising contacting a cell with a therapeutically effective amount of a compound, or a pharmaceutically acceptable salt thereof, as defined herein.

In some embodiments, the inhibitor of EGFR is a compound of the present disclosure.

Effectiveness of compounds of the disclosure can be determined by industry-accepted assays/disease models according to standard practices of elucidating the same as described in the art and are found in the current general knowledge.

The present disclosure also provides a method of treating a disease or disorder in which EGFR is implicated in a subject in need of such treatment, said method comprising administering to said subject a therapeutically effective amount of a compound, or a pharmaceutically acceptable salt thereof, or a pharmaceutical composition as defined herein.

In some embodiments, the subject is a mammal. In some embodiments, the subject is a human.

### Routes of Administration

The compounds of the disclosure or pharmaceutical compositions comprising these compounds may be administered to a subject by any convenient route of administration, whether systemically/peripherally or topically (i.e., at the site of desired action).

Routes of administration include, but are not limited to, oral (e.g. by ingestion); buccal; sublingual; transdermal (including, e.g., by apatch, plaster, etc.); transmucosal (including, e.g., by apatch, plaster, etc.); intranasal (e.g., by nasal spray); ocular (e.g., by eye drops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including subcutaneous, intradermal, intramuscular, intravenous, intra-arterial, intracardiac, intrathecal, intraspinal, intracapsular, subcapsular, intraorbital, intraperitoneal, intratracheal, subcuticular, intraarticular, subarachnoid, and intrastemal; by implant of a depot or reservoir, for example, subcutaneously or intramuscularly.

### EXAMPLES

### General Synthetical Procedures and Examples of the Compound's Preparation.

All reagents were commercial and were used without further purification. Yields refer to puriied and spectroscopically pure compounds. Thin layer chromatography (TLC) was performed using Merck TLC Aluminum sheets silica gel 60 F₂₅₄ plates and visualized by fluorescence quenching under UV light. Flash chromatography was performed using silica gel (Chromatorex, MB 70-40/75, 40-75 m) purchased by Fuji Silysia Chemicals. NMR spectra were recorded on a Varian-400MR operating at 400 MHz for ¹H. Chemical shifts are reported in ppm with the solvent resonance as the internal standard. Data is reported as follows: s=singlet, br=broad, d=doublet, t=triplet, q=quartet, m=multiplet, dd=doublet of doublets; coupling constants in Hz; integration. The purities were recorded on Waters e2695 separations Module/2998 PDA Detector HPLC system (Column: XBridge C_{18, 5} μm, 4.6 mm (ID)×150 mm (L), Eluent: the mixture of mobile phase A and B, mobile phase A: 100% acetonitrile; mobile phase B: pure water containing 0.10% formic acid and 10 mM NH₄0Ac, Flow rate: 0.5 mL/min. detection: UV, 254 nm)

Abbreviations used in the following examples and elsewhere herein are:
ACN acetonitrile
AcOH acetic acid
ATP adenosine triphosphate
anh. anhydrous
aq. aqueous
br. broad
BSA bovine serum albumin
BSTFA N,O-Bis(trimethylsilyl)trifluoroacetamide
d duplet
DCM dichloromethane
DIPEA diisopropylethylamine
DMF N,N-dimethyl formamide
DMSO dimethyl sulfoxide
DTT dithiothreitol
EGFR epidermal growth factor receptor
eq equivalent
FBS fetal bovine serum
g gas
h hour(s)
HPLC high pressure (or performance) liquid chromatography
LAH lithium aluminium hydride
LCMS liquid chromatography mass spectrometry
m multiplet
M molar
MHz megahertz
min minutes
NBS N-bromosuccinimide
NMR nuclear magnetic resonance
PTSA p-Toluenesulfonic acid
rhEGF recombinant human epidermal growth factor
q quadruplet
rt room temperature
s singlet
s solid
sat. saturated
t temperature, triplet
TFA trifluoroacetic acid
THF tetrahydrofuran
TLC thin layer chromatography
TRIS 2-amino-2-(hydroximethyl)propane-1,3-diol

### Synthesis of Building Blocks

### Synthesis of tert-butyl 3-allyl-4-[[3-(2-allyloxyethoxy)-4-pyridyl]methylamino]-5-[(3-fluoro-2-methoxy-phenyl)carbamothioyl]-6-oxo-2,3-dihydropyridine-1-carboxylate (P3)

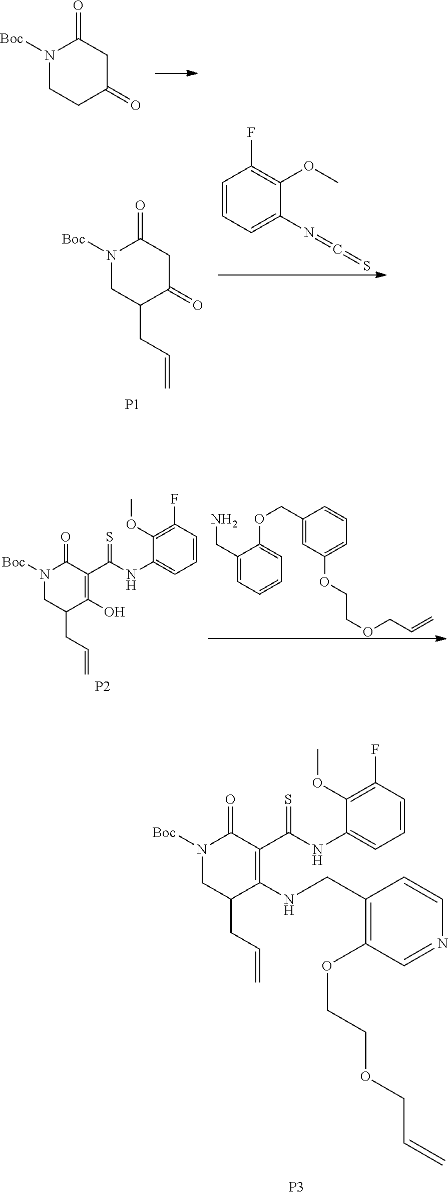

Preparation 1. tert-Butyl 5-allyl-2,4-dioxo-piperidine-1-carboxylate (P1). Intermediate P1 was synthesized in accordance with the procedure described in WO 2021/198020.

Preparation 2. tert-Butyl 3-allyl-5-[(3-fluoro-2-methoxy-phenyl)carbamothioyl]-4-hydroxy-6-oxo-2,3-dihydropyridine-1-carboxylate (P2). Intermediate P2 was synthesized in accordance with the procedure described in WO 2021/198020.

Preparation 3. tert-Butyl 3-allyl-4-[[3-(2-allyloxyethoxy)-4-pyridyl]methylamino]-5-[(3-fluoro-2-methoxy-phenyl)carbamothioyl]-6-oxo-2,3-dihydropyridine-1-carboxylate (P3). To a solution of tert-butyl 5-allyl-3-(3-fluoro-2-methoxyphenylcarbamothioyl)-4-hydroxy-2-oxo-5,6-dihydropyridine-1(2H)-carboxylate (P2, 4.75 g, 7.58 mmol, 1.0 eq) and [3-[[3-(2-allyloxyethoxy)phenyl]methoxy]-4-pyridyl]methanamine (2.37 g, 11.4 mmol, 1.5 eq) in dry acetonitrile (41 mL) N,O-bis(trimethylsilyl)trifluoroacetamide (BSTFA, 6.04 mL, 22.8 mmol, 3.0 eq) was added dropwise at 0° C., then the reaction mixture was stirred at 80° C. for 16 h. The reaction mixture was concentrated, treated with water, and extracted with DCM three times. The combined organic layers were dried over Na₂SO₄(s), filtered, and concentrated in vacuo. The residue was purified by silica gel column chromatography (n-hexane: EtOAc=2:1 to 1:1) to give tert-butyl 5-allyl-4-((3-(2-(allyloxy)ethoxy)pyridin-4-yl)methylamino)-3-(3-fluoro-2-methoxyphenylcarbamothioyl)-2-oxo-5,6-dihydropyridine-1(2H)-carboxylate (P3) (1.93 g, 34%) as a brown foam.

### Synthesis of 7-allyl-2-[3-(2-allyloxyethoxy)-4-pyridyl]-3-(3-fluoro-2-methoxy-anilino)-1,5,6,7-tetrahydropyrrolo[3,2-c]pyridin-4-one (P5)

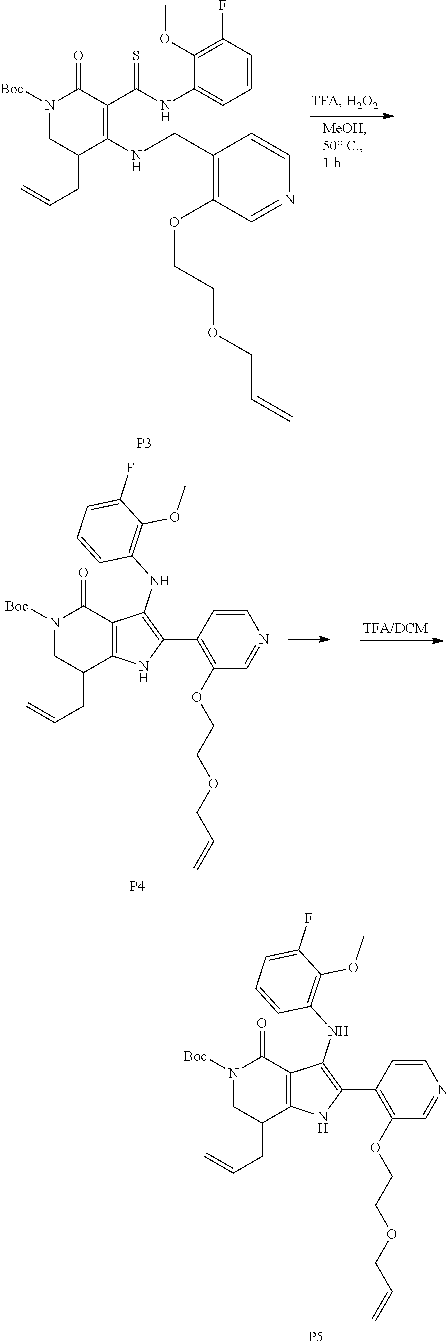

Preparation 4. tert-Butyl 7-allyl-2-[3-(2-allyloxyethoxy)-4-pyridyl]-3-(3-fluoro-2-methoxy-anilino)-4-oxo-6,7-dihydro-1H-pyrrolo[3,2-c]pyridine-5-carboxylate (P4).

To a solution of P3 (1.63 g, 2.60 mmol, 1.0 eq) in methanol (37 mL) TFA (0.20 mL, 2.60 mmol, 1.0 eq) and 30% w/w hydrogen peroxide (0.53 mL, 5.2 mmol, 2.0 eq) were added dropwise at 0° C. The reaction mixture was stirred at 50° C. for 1 h, concentrated and treated with aqueous saturated solution of sodium bicarbonate to adjust pH=7. The mixture was extracted with DCM three times. The combined organic layers were dried over Na₂SO₄(s), filtered, and concentrated in vacuo to give a residue. The residue was purified by C-18 reversed-phase column chromatography (mobile phase A: water (with 0.1% NH₄HCO₃), mobile phase B: methanol, UV: 214 and 254 nm, flow rate: 60 mL/min, gradient: 3-100% (% B)) to give tert-butyl 7-allyl-2-(3-(2-(allyloxy)ethoxy)pyridin-4-yl)-3-(3-fluoro-2-methoxyphenylamino)-4-oxo-6,7-dihydro-1H-pyrrolo[3,2-c]pyridine-5(4H)-carboxylate (P4) (909 mg, 59% yield) as a yellow foam.

Preparation 5. 7-Allyl-2-[3-(2-allyloxyethoxy)-4-pyridyl]-3-(3-fluoro-2-methoxy-anilino)-1,5,6,7-tetrahydropyrrolo[3,2-c]pyridin-4-one (P5).

To a solution of crude P4 (419 mg, 0.707 mmol, 1.0 eq) in DCM (5.0 mL) TFA (2.5 mL) was added dropwise. The reaction mixture was stirred at rt for 3 h. The solution was treated with a saturated aqueous solution of sodium bicarbonate until pH=7 and extracted with DCM three times. The combined organic layers were dried over Na₂SO₄(s), filtered, and concentrated in vacuo. The residue was purified by C-18 reversed-phase column chromatography (mobile phase A: water (with 0.1% NH₄HCO₃), mobile phase B: methanol, UV: 214 and 254 nm, Flow rate: 35 mL/min, Gradient: 3-100% (% B)) to give 7-allyl-2-(3-(2-(allyloxy)ethoxy)pyridin-4-yl)-3-(3-fluoro-2-methoxyphenylamino)-6,7-dihydro-1H-pyrrolo[3,2-c]pyridin-4(5H)-one (P5) (213 mg, 61% yield) as a yellow foam.

### Synthesis of [3-[(3-allyloxyphenyl)methoxy]-4-pyridyl]methanamine (P10)

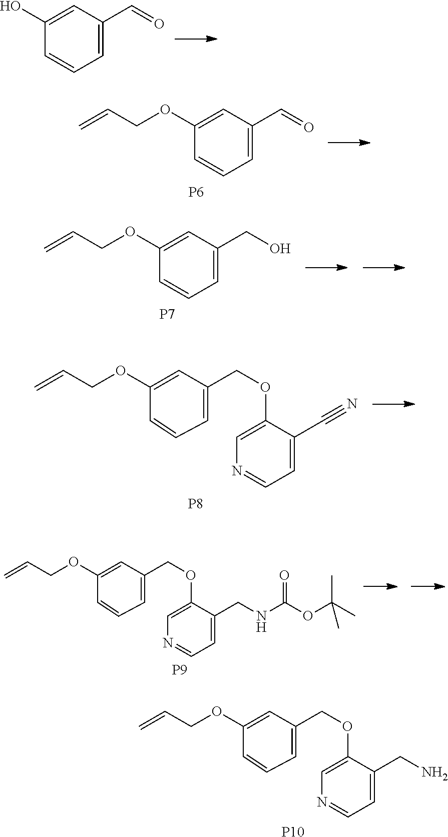

### Preparation 6. 3-Allyloxybenzaldehyde (P6)

To a mixture of 3-hydroxybenzaldehyde (17 g, 0.14 mol, 1 eq) in MeCN (V=150 mL) was added K₂CO₃ (39 g, 0.28 mol, 2 eq) and mixture was stirred at rt for 5 min. 3-bromoprop-1-ene (18.5 g, 0.153 mol, 1.1 eq) was added and the mixture was stirred at 70° C. for 17 h. TLC (CHCl₃:MeOH=19:1). Reaction mixture was rotary evaporated to dryness, EtOAc (100 mL) and water (200 mL) were added to the residue and the mixture was stirred at rt for 5 min. Organic layer was separated, and aqueous layer was extracted with EtOAc (2×100 mL). Combined extract was washed with brine, dried over Na₂SO₄ and rotary evaporated to give 22 g, 97%. ¹H NMR (400 MHz, DMSO-d₆), δ: 9.97 (s, 1H), 7.57-7.48 (m, 2H), 7.44 (d, J=1.8 Hz, 1H), 7.35-7.25 (m, 1H), 6.13-5.98 (m, 1H), 5.41 (dd, J=17.3, 1.7 Hz, 1H), 5.28 (dd, J=10.5, 1.5 Hz, 1H), 4.69-4.61 (m, 2H).

### Preparation 7. (3-Allyloxyphenyl)methanol (P7)

To a solution of 3-(allyloxy)benzaldehyde (P6, 22 g, 0.14 mol, 1 eq) in MeOH (220 mL) (water bath, rt) NaBH4 (5.1 g, 0.13 mol, 1 eq) was added portionwise and resulted mixture was stirred at rt for 10 min. TLC (CHCl₃:MeOH=19:1) The mixture was rotary evaporated, saturated solution ofNaHCO₃ (150 mL) was added to the residue and the product was extracted with EtOAc (3×100 mL). Combined extract was washed with brine, dried over Na₂SO₄ and rotary evaporated to dryness to give P7 (21.9 g, 98%). ¹H NMR (400 MHz, DMSO-d₆), δ: 7.22 (t, J=7.8 Hz, 1H), 6.95-6.84 (m, 2H), 6.80 (d, J=8.1 Hz, 1H), 6.12-5.96 (m, 1H), 5.44-5.34 (m, 1H), 5.29-5.21 (m, 1H), 5.19-5.12 (m, 1H), 4.58-4.51 (m, 2H), 4.47 (d, J=5.6 Hz, 2H).

### Preparation 8. 3-[(3-Allyloxyphenyl)methoxy]pyridine-4-carbonitrile (P8)

To a suspension of NaH (60% in oil, 3.03 g, 75.8 mmol, 1.5 eq) in DMF (200 mL) at −10-0° C. solution of [3-(allyloxy)phenyl]methanol (P7, 9.13 g, 55.6 mmol, 1.1 eq) in DMF (15 mL) was added dropwise. The mixture was stirred at rt for 30 min. 3-chloroisonicotinonitrile (7 g, 50.5 mmol, 1 eq) in DMF (30 mL) was added to the reaction mixture at rt and stirring was continued for 17 h at rt. TLC (Hexane:EtOAc=2:1). Saturated solution ofNH₄C₁ (10 mL) was added and the mixture was rotary evaporated to dryness. The residue was suspended in water (200 mL) and EtOAc (100 mL) mixture, extracted with EtOAc (3×100 mL). Combined extract was washed with water (40 mL), brine, dried over Na₂SO₄ and rotary evaporated to dryness. The product was purified by column chromatography (silica gel, Hexane:EtOAc=4:1-2:1-1:1) to afford P8 (9.1 g, 68%). ¹H NMR (400 MHz, DMSO-d₆), δ: 8.76 (s, 1H), 8.38 (d, J=6.5 Hz, 1H), 7.79 (d, J=6.8 Hz, 1H), 7.34 (t, J=7.9 Hz, 1H), 7.12-7.00 (m, 2H), 6.95 (dd, J=8.2, 2.3 Hz, 1H), 6.12-5.96 (m, 1H), 5.46-5.33 (m, 3H), 5.25 (d, J=10.5 Hz, 1H), 4.58 (d, J=5.2 Hz, 2H).

### Preparation 9. tert-ButylN-[[3-[(3-allyloxyphenyl)methoxy]-4-pyridyl]methyl]carbamate (P9)

To a suspension of LAH (3.72 g, 98.1 mmol, 3 eq) in THF (800 mL) (water bath, rt) a solution of 3-{[3-(allyloxy)benzyl]oxy}isonicotinonitrile (P8, 8.7 g, 32.7 mmol, 1 eq) in THF (100 mL) was added dropwise and mixture was stirred at rt for 17 h. TLC (CHCl₃:MeOH:NH₄0H(25% aq.)=100:10:1). Water (3.7 mL) was added dropwise to reaction mixture (ice-water bath) followed by careful addition of aq. solution of NaOH (1.2 g in 3.7 mL of water). Then reaction mixture was stirred at rt for 15 min. The solid was filtered and washed with THF (2x). The filtrate was rotary evaporated to dryness. The residue was dissolved in DCM (100 mL) and Et₃N (5 g, 48.9 mmol, 1.5 eq) and solution of Boc₂0 (7.1 g, 32.6 mmol, 1 eq) in DCM (20 mL) were added. The mixture was stirred at rt for 1 h and rotary evaporated to dryness. TLC (CHCl₃:MeOH=19:1). The product was purified by column chromatography (silica gel, Hexane:EtOAc=3:1-2:1-1:1-0:1) to give 4.7 g, 39%. ¹H NMR (400 MHz, DMSO-d₆), δ: 8.32 (s, 1H), 8.18 (d, J=4.7 Hz, 1H), 7.40-7.27 (m, 2H), 7.15 (d, J=4.6 Hz, 1H), 7.09-7.01 (m, 2H), 6.92 (dd, J=8.1, 2.2 Hz, 1H), 6.11-5.97 (m, 1H), 5.43-5.35 (m, 1H), 5.28-5.21 (m, 3H), 4.58 (d, J=5.2 Hz, 2H), 4.19 (d, J=6.0 Hz, 2H), 1.40 (s, 9H).

### Preparation 10. [3-[(3-Allyloxyphenyl)methoxy]-4-pyridyl]methanamine (P10)

To a solution of tert-butyl [(3-{[3-(allyloxy)benzyl]oxy}pyridin-4-yl)methyl]carbamate (P9, 2.2 g, 5.8 mmol, 1 eq) in DCM (33 mL) CF₃COOH (13 mL) was added at 0° C. The mixture was stirred at rt for 20 mn and rotary evaporated to dryness. TLC (CHCl₃:MeOH:NH₄0H(25% aq.)=100:10:1). The residue was treated with DCM (50 mL) and sat. sol. of NaHCO₃, extracted with DCM (3×50 mL). Combined extract was washed with brine, dried over Na₂SO₄ and rotary evaporated to dryness to give 1.5 g (91%) of P10. ¹H NMR (400 MHz, DMSO-d₆), δ: 8.29 (s, 1H), 8.23-8.14 (m, 1H), 7.47-7.38 (m, 1H), 7.37-7.26 (m, 1H), 7.11-7.00 (m, 2H), 6.92 (d, J=8.2 Hz, 1H), 6.12-5.98 (m, 1H), 5.39 (d, J=17.2 Hz, 1H), 5.31-5.17 (m, 3H), 4.65-4.54 (m, 2H), 3.75 (s, 2H), 1.93 (br. s., 2H).

### Synthesis of 7-allyl-2-[3-[(3-allyloxyphenyl)methoxy]-4-pyridyl]-3-(3-fluoro-2-methoxy-anilino)-1,5,6,7-tetrahydropyrrolo[3,2-c]pyridin-4-one (P12)

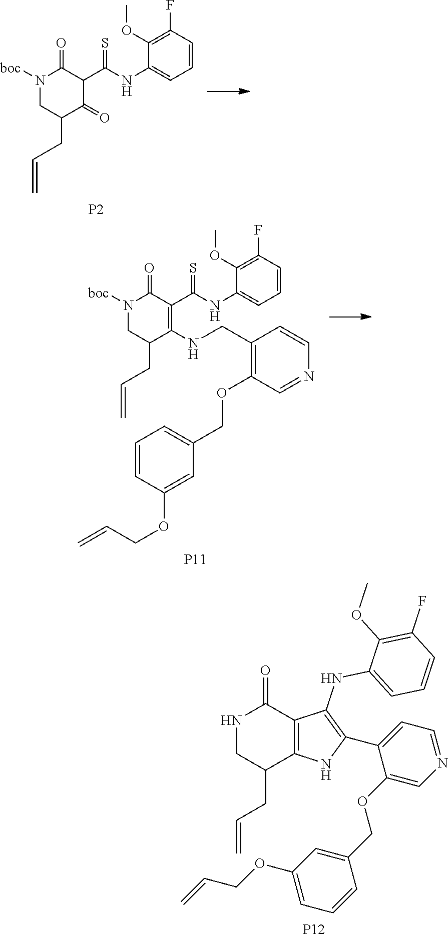

Preparation 11. tert-Butyl 3-allyl-4-{[(3-{[3-(allyloxy)benzyl]oxy}pyridin-4-yl)methyl]amino}-5-{[(3-fluoro-2-methoxyphenyl)amino]carbonothioyl}-6-oxo-3,6-dihydropyridine-1(2H)-carboxylate (P11).

BSTFA (2.5 g, 9.6 mmol, 3 eq) was added dropwise at 0° C. to a solution of tert-butyl 5-allyl-3-{[(3-fluoro-2-methoxyphenyl)amino]carbonothioyl}-2,4-dioxopiperidine-1-carboxylate (P2, 1.4 g, 3.2 mmol, 1.0 eq) and [(3-{[3-(allyloxy)benzyl]oxy}pyridin-4-yl)methyl]amine (P10, 1 g, 3.8 mmol, 1.2 eq) in toluene (14 mL) and reaction mixture was stirred under argon at 80° C. for 48 h. TLC (100% EtOAc). The reaction mixture was poured in mixture of sat. sol. of NaHCO₃ and EtOAc. The product was extracted with EtOAc (2×60 mL). Combined extract was washed with brine, dried over Na₂SO₄ and rotary evaporated to dryness. The product was purified by column chromatography (silica gel, Hexane:EtOAc=2:1-1:1) to afford P11 (440 mg, 20%). ¹H NMR (400 MHz, DMSO-d₆), δ: 13.63 (s, 1H), 13.49 (s, 1H), 8.46 (s, 1H), 8.25 (d, J=4.5 Hz, 1H), 7.58-7.50 (m, 1H), 7.43 (d, J=4.7 Hz, 1H), 7.29 (t, J=7.9 Hz, 1H), 7.17-7.02 (m, 4H), 6.95-6.89 (m, 1H), 6.07-5.97 (m, 1H), 5.83-5.70 (m, 1H), 5.41-5.33 (m, 1H), 5.29-5.20 (m, 3H), 5.11 (d, J=10.1 Hz, 1H), 5.01 (d, J=17.2 Hz, 1H), 4.85-4.67 (m, 2H), 4.61-4.41 (m, 3H), 3.78 (d, J=14.8 Hz, 4H), 3.20-3.11 (m, 1H), 2.32-2.23 (m, 1H), 2.15-2.05 (m, 1H), 1.46 (s, 9H).

Preparation 12. 7-Allyl-2-(3-{[3-(allyloxy)benzyl]oxy}pyridin-4-yl)-3-[(3-fluoro-2-methoxyphenyl)amino]-1,5,6,7-tetrahydro-4H-pyrrolo[3,2-c]pyridin-4-one (P12). To a solution of tert-butyl 3-allyl-4-{[(3-{[3-(allyloxy)benzyl]oxy}pyridin-4-yl)methyl]amino}-5-{[(3-fluoro-2-methoxyphenyl)amino]carbonothioyl}-6-oxo-3,6-dihydropyridine-1(2H)-carboxylate (P11, 440 mg, 0.639 mmol, 1 eq) in methanol (5 mL) TFA (72.5 mg, 0.636 mol, 0.996 eq), 2-tert-butylphenole (96 mg, 0.639 mmol, 1 eq) and hydrogen peroxide (117.4 mg, 37%, 1.28 mmol, 2 eq) were added. Resulted mixture was stirred at rt for 5 min then was heated in CEM MW system at 100° C. for 30 min. TLC (100% EtOAc). The reaction mixture was quenched with saturated sodium bisulfite solution and sodium bicarbonate solution and extracted with EtOAc (3×60 mL). The organic layer was dried and concentrated under reduced pressure. The product was purified by column chromatography (silica gel, Hexane:EtOAc=2:1-1:1-1:2-0:1) to afford P12 (140 mg, 39%). ¹H NMR (400 MHz, DMSO-d₆), δ: 11.95 (s, 1H), 11.12 (s, 1H), 8.42 (s, 1H), 8.08-8.00 (m, 1H), 7.42 (s, 1H), 7.33-7.25 (m, 2H), 7.11-7.00 (m, 3H), 6.94-6.88 (m, 1H), 6.60-6.52 (m, 1H), 6.48-6.39 (m, 1H), 6.06-5.94 (m, 2H), 5.90-5.75 (m, 1H), 5.39-5.20 (m, 4H), 5.14-5.05 (m, 2H), 4.55-4.48 (m, 2H), 3.82 (s, 3H), 3.49-3.41 (m, 1H), 3.20-3.12 (m, 1H), 3.10-3.00 (m, 1H), 2.37-2.19 (m, 1H).

### Synthesis of 7-allyl-3-[(3-fluoro-2-methoxyphenyl)amino]-2-{3-[(1-hex-5-enoylpiperidin-3-yl)methoxy]pyridin-4-yl}-1,5,6,7-tetrahydro-4H-pyrrolo[3,2-c]pyridin-4-one (P16)

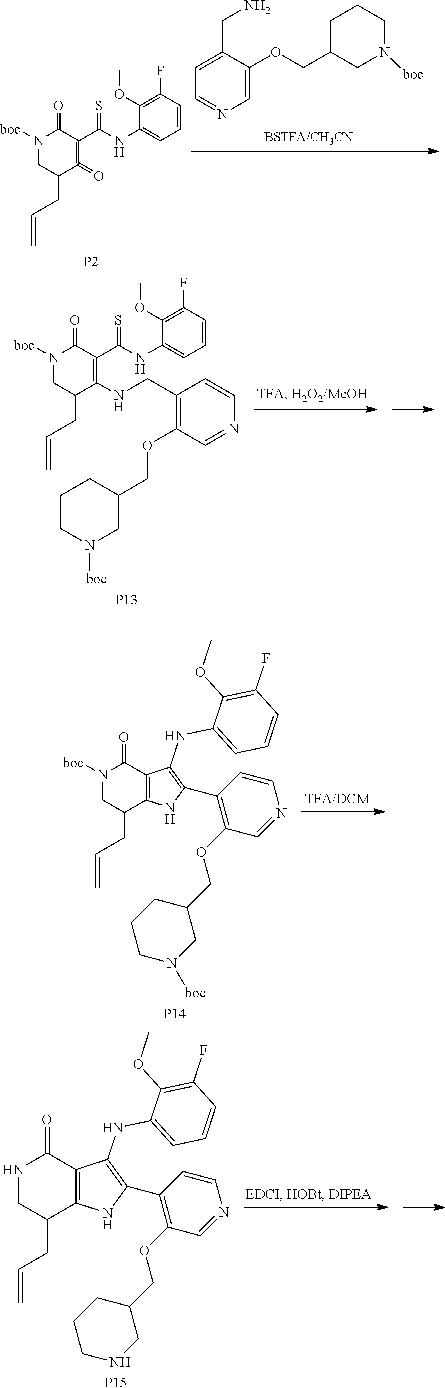

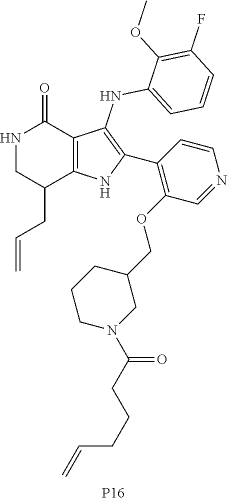 [SMILES: C=CCCCC(=O)N1CCCC(COc2cnccc2-c2[nH]c3c(c2Nc2cccc(F)c2OC)C(=O)NCC3CC=C)C1 ; "P16"]

Preparation 13. tert-Butyl 3-allyl-4-{[(3-{[1-(tert-butoxycarbonyl)piperidin-3-yl]methoxy}pyridin-4-yl)methyl]amino}-5-{[(3-fluoro-2-methoxyphenyl)amino]carbonothioyl}-6-oxo-3,6-dihydropyridine-1(2H)-carboxylate (P13).

To a mixture of tert-butyl 5-allyl-3-(3-fluoro-2-methoxyphenylcarbamothioyl)-4-hydroxy-2-oxo-5,6-dihydropyridine-1(2H)-carboxylate P2 (1.0 g, 2.3 mmol, 1.0 eq), tert-butyl 3-({[4-(aminomethyl)pyridin-3-yl]oxy}methyl)piperidine-1-carboxylate (0.88 g, 2.7 mmol, 1.2 eq) and molecular sieves (4A, 1 g) in dry ACN (10 mL) N,O-bis(trimethylsilyl)trifluoroacetamide (BSTFA, 1.8 mL, 6.9 mmol, 3.0 eq) was added dropwise at 0° C., and then the reaction mixture was stirred at 80° C. for 16 h. The solid was removed by filtration and the solution was concentrated, treated with water, and extracted with DCM three times. The combined organic layers were dried over Na₂SO₄, filtered, and concentrated in vacuo. The residue was purified by silica gel column chromatography (DCM:EtOAc 0-50%) to give P13 (1.47 g, 87%) as a yellow oil. LCMS (ESI+) m z: 740 [M+H]+.

### Preparation 14. tert-Butyl 7-allyl-2-(3-{[1-(tert-butoxycarbonyl)piperidin-3-yl]methoxy}pyridin-4-yl)-3-[(3-fluoro-2-methoxyphenyl)amino]-4-oxo-1,4,6,7-tetrahydro-5H-pyrrolo[3,2-c]pyridine-5-carboxylate (P14)

To a solution of P13 (1.47 g, 1.98 mmol, 1.0 eq) in methanol (40 mL)trifluoroacetic acid (0.15 mL, 1.98 mmol, 1.0 eq) and 35% w/w hydrogen peroxide (0.38 mL, 3.97 mmol, 2.0 eq) were added dropwise at 0° C. The reaction mixture was stirred at 50° C. for 1 h, concentrated and treated with saturated aqueous solution of sodium bicarbonate to adjust pH=7. The mixture was extracted with DCM three times. The combined organic layers were dried over Na₂SO₄, filtered, and concentrated in vacuo to give P14 (900 mg, 64%) as a yellow foam. ¹H NMR (400 MHz, DMSO-d₆), δ: 11.95 (s, 1H), 11.44 (s, 1H), 8.41 (s, 1H), 8.11 (s, 1H), 7.28 (t, J=5.1 Hz, 1H), 7.19 (d, J=8.6 Hz, 1H), 6.59 (s, 1H), 6.47-6.34 (m, 1H), 6.03 (t, J=8.0 Hz, 1H), 5.84 (d, J=6.9 Hz, 1H), 5.18-5.00 (m, 2H), 3.96-3.92 (m, 5H), 3.85 (s, 3H), 3.76 (d, J=19.2 Hz, 2H), 3.17 (s, 1H), 2.90-2.63 (m, 2H), 2.56 (s, 1H), 2.28 (s, 1H), 1.79 (s, 1H), 1.61 (s, 1H), 1.46-1.39 (m, 9H), 1.34 (s, 13H). LCMS (ESI+) m/z: 706 [M+H]+.

### Preparation 15. 7-Allyl-3-[(3-fluoro-2-methoxyphenyl)amino]-2-[3-(piperidin-3-ylmethoxy)pyridin-4-yl]-1,5,6,7-tetrahydro-4H-pyrrolo[3,2-c]pyridin-4-one (P15)

To a solution of P14 (900 mg, 1.27 mmol, 1.0 eq) in DCM (10.0 mL)trifluoroacetic acid (4.5 mL) was added dropwise. The reaction mixture was stirred at rt for 3 h. The solution was treated with a sat. aq. solution of sodium bicarbonate until pH=7 and extracted with DCM three times. The combined organic layers were dried over Na₂SO₄, filtered, and concentrated in vacuo to give P15 (500 mg, 78%) as an orange solid. ¹H NMR (400 MHz, DMSO-d₆), δ: 11.36-11.32 (m, 1H), 8.74 (s, 1H), 8.52 (d, J=4.1 Hz, 2H), 8.28 (d, J=5.7 Hz, 1H), 7.82 (s, 1H), 7.54 (d, J=3.3 Hz, 1H), 7.24 (s, 1H), 6.65-6.61 (m, 1H), 6.58-6.46 (m, 1H), 5.98-5.75 (m, 2H), 5.21-5.08 (m, 3H), 4.18-3.98 (m, 3H), 3.50-3.41 (m, 1H), 3.26-3.22 (m, 4H), 2.84-2.65 (m, 2H), 2.57-2.53 (m, 1H), 2.34 (s, 2H), 1.85-1.82 (m, 2H), 1.65-1.60 (m, 1H), 1.42-1.19 (m, 1H). MS (ESI+) m/z: 506 [M+H]+.

### Preparation 16. 7-Allyl-3-[(3-fluoro-2-methoxyphenyl)amino]-2-{3-[(1-hex-5-enoylpiperidin-3-yl)methoxy]pyridin-4-yl}-1,5,6,7-tetrahydro-4H-pyrrolo[3,2-c]pyridin-4-one (P16)

To solution of P15 (150 mg, 0.3 mmol, 1 eq), DIPEA (0.08 mL, 0.45 mmol, 1.5 eq), hex-5-enoic acid (37 mg, 0.33 mmol, 1.1 eq), HOBt (40 mg, 0.3 mmol, 1 eq) in DCM (2 mL) EDAC (75 mg, 0.39 mmol) was added. Then the reaction mixture was stirred at rt for overnight. Then reaction mixture was treated with water and extracted with DCM three times. The combined organic layers were dried over Na₂SO₄, filtered, and concentrated in vacuo. The residue was purified by silica gel column chromatography (EtOAc:MeOH 0-10%) to give P16 (125 mg, 87%) as a yellow oil. ¹H NMR (400 MHz, DMSO-d₆), δ: 11.94 (s, 1H), 11.15 (d, J=9.7 Hz, 2H), 8.39 (d, J=7.0 Hz, 2H), 8.09 (d, J=4.8 Hz, 1H), 8.02 (d, J=2.9 Hz, 1H), 7.47 (d, J=11.6 Hz, 1H), 7.41 (s, 1H), 7.33-7.19 (m, 2H), 7.05 (s, 2H), 6.66-6.52 (m, 2H), 6.47-6.35 (m, 2H), 6.04-5.93 (m, 2H), 5.78-5.75 (in, 4H), 5.17-5.03 (m, 4H), 4.93-4.88 (m, 4H), 4.21-3.91 (m, 7H), 3.85 (t, J=13.9 Hz, 7H), 3.57 (s, 1H), 3.48-3.44 (m, 2H), 3.23-2.94 (m, 6H), 2.41-2.21 (m, 5H), 2.21-2.08 (m, 2H), 2.02-1.98 (m, 4H), 1.89-1.77 (m, 4H), 1.72-1.52 (m, 5H), 1.50-1.39 (m, 4H), 1.30-1.23 (m, 3H). MS (ESI+) m/z: 602 [M+H]+.

### Synthesis of 19-[(3-fluoro-2-methoxyphenyl)amino]-8-methyl-6,7,8,9,12,12a,13,14-octahydro-16,18-methenodipyrido[3,4-i:4′,3′-m][1,4,11]oxadiazacyclotetradecin-15(17H)-one (P21)

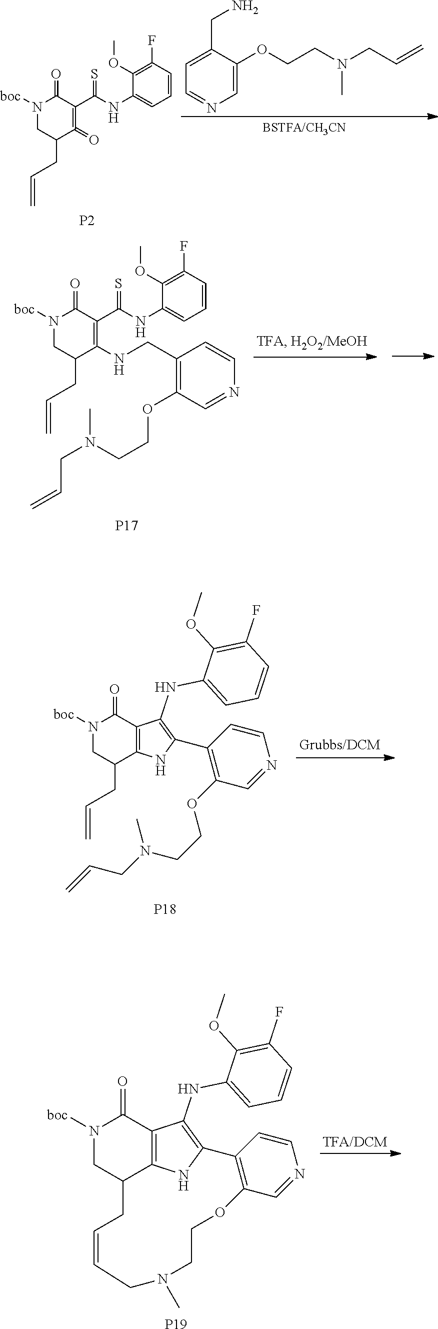

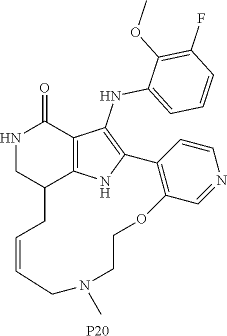

### Preparation 17. tert-Butyl 3-allyl-4-{[(3-{2-[allyl(methyl)amino]ethoxy}pyridin-4-yl)methyl]amino}-5-{[(3-fluoro-2-methoxyphenyl)amino]carbonothioyl}-6-oxo-3,6-dihydropyridine-1(2H)-carboxylate (P17)

To a solution of P2 (1.0 g, 2.3 mmol, 1.0 eq), N-(2-{[4-(aminomethyl)pyridin-3-yl]oxy}ethyl)-N-methylprop-2-en-1-amine (0.6 g, 2.7 mmol, 1.2 eq) and molecular sieves (4A, 1 g) in dry acetonitrile (10 mL) N,O-bis(trimethylsilyl)trifluoroacetamide (BSTFA, 1.8 mL, 6.9 mmol, 3.0 eq) was added dropwise at 0° C., and then the reaction mixture was stirred at 80° C. for 16 h. The solid was removed by filtration and the solution was concentrated, treated with water, and extracted with DCM three times. The combined organic layers were dried over Na₂SO₄, filtered, and concentrated in vacuo. The residue was purified by silica gel column chromatography (DCM: MeOH 0-10%) to give P17 (1.1 g, 75% yield) as a yellow oil. LCMS (ESI+) m/z 640 [M+H]⁺. ¹H NMR (400 MHz, DMSO), δ: 13.59 (s, 1H), 13.37 (s, 1H), 8.42 (s, 1H), 8.24 (d, J=4.7 Hz, 1H), 7.57 (d, J=7.2 Hz, 1H), 7.40 (d, J=4.8 Hz, 1H), 7.22-6.99 (m, 2H), 5.96-5.70 (m, 2H), 5.22-4.99 (m, 4H), 4.71 (s, 2H), 4.26 (d, J=3.6 Hz, 2H), 3.92-3.74 (m, 4H), 3.46 (d, J=12.4 Hz, 1H), 3.17 (s, 1H), 3.05 (d, J=6.1 Hz, 2H), 2.77 (t, J=5.7 Hz, 2H), 2.38-2.12 (m,, 5H), 1.46 (s, 9H).

### Preparation 18. tert-Butyl 7-allyl-2-(3-{2-[allyl(methyl)amino]ethoxy}pyridin-4-yl)-3-[(3-fluoro-2-methoxyphenyl)amino]-4-oxo-1,4,6,7-tetrahydro-5H-pyrrolo[3,2-c]pyridine-5-carboxylate (P18)

To a solution of P17 (1.1 g, 1.7 mmol, 1.0 eq) in methanol (40 mL)trifluoroacetic acid (0.13 mL, 1.7 mol, 1.0 eq) and 35% w/w hydrogen peroxide (0.33 mL, 3.44 mmol, 2.0 eq) were added dropwise at 0° C. The reaction mixture was stirred at 50° C. for 1 h, concentrated and treated with aq. sat. solution of sodium bicarbonate to adjust pH=7. The mixture was extracted with DCM three times. The combined organic layers were dried over Na₂SO₄, filtered, and concentrated in vacuo. The residue was purified by silica gel column chromatography (DCM: EtOAc 0-100, EtOAc: MeOH 0-10%) to give P18 (500 mg, 48%) as a yellow foam. LCMS (ESI+) m/z 606 [M+H]⁺.

### Preparation 19. tert-Butyl 19-[(3-fluoro-2-methoxyphenyl)amino]-8-methyl-15-oxo-7,8,9,12,12a,13,15,17-octahydro-16,18-methenodipyrido[3,4-i:4′,3′-m][1,4,11]oxadiazacyclotetradecine-14(6H)-carboxylate (P19)

To a solution of P18 (150 mg, 0.25 mmol, 1.0 eq) and Ti(iOPr)₄ (14 mg, 0.05 mmol, 0.2 eq) in toluene (1.0 mL) Grubbs (5 mg) was added. The reaction mixture was reflux for 3 h, concentrated in vacuo. The residue after evaporation was purified by silica gel column chromatography (EtOAc: MeOH 0-10%) to give P19. LCMS (ESI⁺) m/z 578 [M+H]⁺. ¹H NMR (400 MHz, CDCl₃), δ: 10.54 (s, 8H), 8.29 (s, 7H), 8.01 (s, 8H), 7.55 (s, 7H), 7.46 (s, 7H), 6.42-6.65 (m, 2H), 6.09-5.87 (m, 3H), 4.17-4.38 (m, 3H), 4.12 (s, 3H), 3.66-3.36 (m, 5H), 3.24-3.08 (m, 2H), 3.09-2.90 (m, 1H), 2.78-2.63 (m, 1H), 2.57-2.35 (m, 5H), 1.55 (s, 9H).

### Preparation 20. 19-[(3-Fluoro-2-methoxyphenyl)amino]-8-methyl-6,7,8,9,12,12a,13,14-octahy dro-16,18-methenodipyrido[3,4-i:4′,3′-m][1,4,11]oxadiazacyclotetradecin-15(17H)-one (P20)

To a solution of P19 (100 mg, 0.17 mmol, 1.0 eq) in DCM (1.0 mL)trifluoroacetic acid (0.1 mL) was added dropwise. The reaction mixture was stirred at rt for 3 h. The solution was treated with a sat. aq. solution of sodium bicarbonate until pH=7 and extracted with DCM three times. The combined organic layers were dried over Na₂SO₄, filtered and concentrated in vacuo to give P20 (80 mg, 97%) as an orange solid. LCMS (ESI⁺) m/z 478 [M+H]⁺.

### Synthesis of 1³-((3-fluoro-2-methoxyphenyl)amino)-1⁴,1⁵,1⁶,1⁷-tetrahydro-1¹H-3-oxa-1(2,7)-pyrrolo[3,2-c]pyridina-2(4,3)-pyridinacyclooctaphan-6-en-1⁴-one (P26)

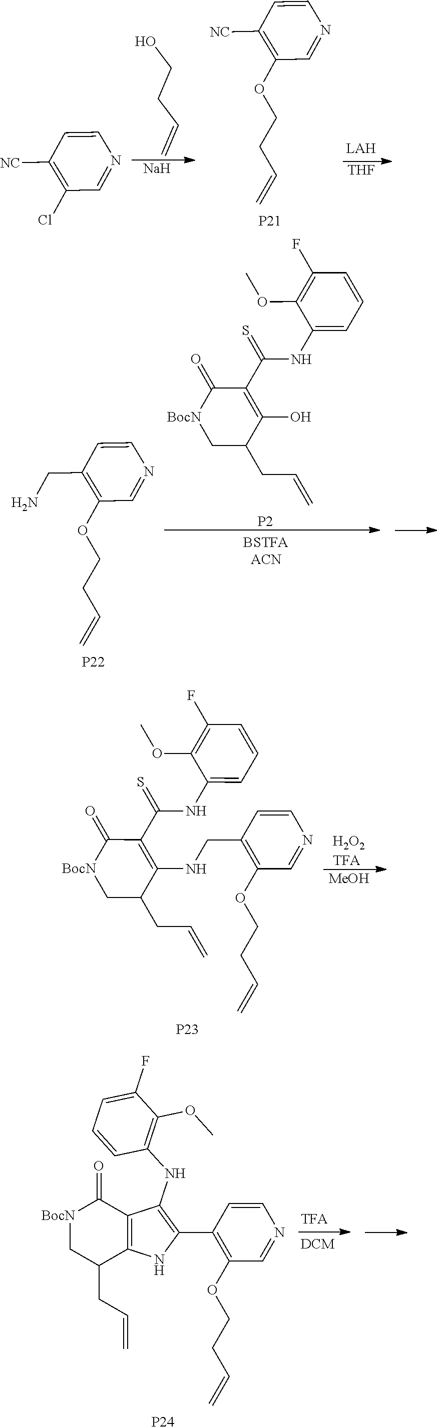

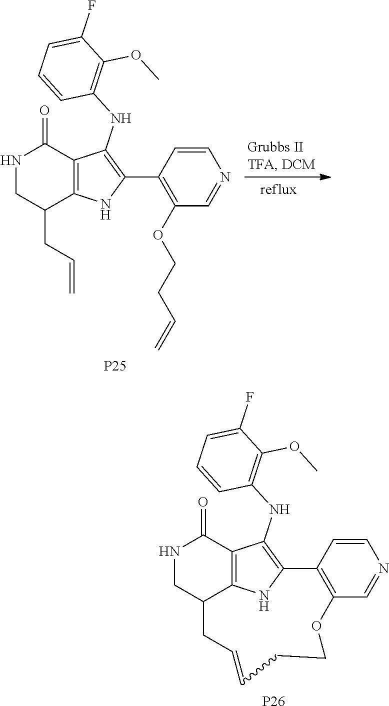

### Preparation 21. 3-(But-3-en-1-yloxy)isonicotinonitrile (P21)

To a solution of but-3-en-1-ol (3.1 g, 43 mmol) in THF (140 mL) was added NaH (1.7 g, 42 mmol) at 0° C. and it was stirred at 0° C. for 30 min. 3-Chloropyridine-4-carbonitrile (5.0 g, 36 mmol) was added to this solution at 0° C. and the reaction was stirred at rt for 4 h. The mixture was treated with NH₄C₁(_{aq}) and extracted with EtOAc. The organic layers were dried over Na₂SO₄(s), filtered, and concentrated under reduced pressure. The crude was purified by silica-gel column chromatography (0-50% EtOAc in n-hexane) to give P21 (5.1 g, 81%) as a yellow solid. LCMS (ESI) m/z calc. for C₁₀H₁₀N₂O 174.08; found, 174.9 [M+H]⁺.

### Preparation 22. (3-(But-3-en-I-yloxy)pyridin-4-yl)methanamine (P22)

To a solution of P21 (5.1 g, 3.44 mmol) in THF (60 mL) was added LAH (1 M in THF, 32 mL, 32 mmol) at 0° C. The mixture was stirred at rt for 5 h. The solution was treated with NaOH(_{aq}).

The mixture was filtered and washed with EtOAc. The filtrate was concentrated. The crude was puriied by C₁₈ column chromatography (0-100% methanol in H₂O) to give P22 (3.0 g, 58%), as a yellow oil. ¹H NMR (400 MHz, CD₃0D), 6:8.20 (s, 1H), 8.14 (d, J=4.8 Hz, 1H), 7.36 (d, J=4.8 Hz, 1H), 6.03-5.87 (m, 1H), 5.25-5.15 (m, 1H), 5.15-5.09 (m, 1H), 4.21 (t, J=6.2 Hz, 2H), 3.81 (s, Hz, 2H), 2.65-2.54 (m, 2H); LCMS (ESI) m/z calc. for CioH₁₄N20 178.11; found, 179.0 [M+H]⁺.

### Preparation 23. tert-Butyl 3-allyl-4-(((3-(but-3-en-1-yloxy)pyridin-4-yl)methyl)amino)-5-((3-fluoro-2-methoxyphenyl)carbamothioyl)-6-oxo-3,6-dihydropyridine-1(2H)-carboxylate (P23)

To a solution of P2 (1.50 g, 3.44 mmol) in anh. acetonitrile (11 mL) was added P22 (919 mg, 5.15 mmol) and N,O-bis(trimethylsilyl)trifluoroacetamide (BSTFA, 2.7 mL, 10 mmol). The mixture was stirred at 80° C. for overnight. The solution was concentrated to remove the solvent. The crude was puriied by silica-gel column chromatography (0-10% methanol in DCM) to give P23 (2.0 g, 98%), as a yellow solid. ¹H NMR (400 MHz, CDCl₃), δ: 13.72 (br. s, 1H), 8.26 (s, 1H), 8.25 (d, J=4.8 Hz, 1H), 7.52-7.43 (m, 1H), 7.29 (d, J=4.8 Hz, 1H), 7.03-6.94 (m, 2H), 5.92-5.80 (m, 1H), 5.80-5.68 (m, 1H), 5.22-5.07 (m, 4H), 4.67-4.50 (m, 2H), 4.18 (t, J=6.6 Hz, 2H), 4.07-4.04 (m, 1H), 3.94 (s, 3H), 3.38-3.36 (m, 1H), 2.76-2.66 (m, 1H), 2.63-2.54 (m, 2H), 2.49-2.38 (m, 1H), 2.35-2.23 (m, 1H), 1.51 (s, 9H); LCMS (ESI) m/z calc. for C₃₁H3₇FN₄05S 596.25; found, 597.3 [M+H]⁺.

### Preparation 24. tert-Butyl 7-allyl-2-(3-(but-3-en-1-yloxy)pyridin-4-yl)-3-((3-fluoro-2-methoxyphenyl)amino)-4-oxo-1,4,6,7-tetrahydro-5H-pyrrolo[3,2-c]pyridine-5-carboxylate (P24)

To a solution of P23 (2.0 g, 3.3 mmol) in methanol (17 mL) was added TFA (0.25 mL, 3.30 mmol) and hydrogen peroxide (30% in water, 0.76 mL, 6.7 mmol). The mixture was stirred at 50° C. for 1 h. The solution was concentrated. The crude (1.6 g) was used for the next step without further puriication. LCMS (ESI) m/z calc. for C₃₁H₃₅FN₄O₅ 562.26; found, 563.4 [M+H]⁺.

### Preparation 25. 7-Allyl-2-(3-(but-3-en-1-yloxy)pyridin-4-yl)-3-((3-fluoro-2-methoxyphenyl)amino)-1,5,6,7-tetrahydro-4H-pyrrolo[3,2-c]pyridin-4-one (P25)

A mixture of P24 (1.60 g, 2.80 mmol) and TFA (6.0 mL) in DCM (12 mL) was stirred at rt for 1 h. The solution was concentrated to remove solvent. The crude was purified by silica-gel column chromatography (0-20% methanol in DCM) and C₁₈ column chromatography (10-100% methanol in H₂O) to give P25 (1.10 g, impure) as a yellow solid. ¹H NMR (400 MHz, CDCl₃), δ: 10.07 (br. s, 1H), 8.70 (s, 1H), 8.11 (s, 1H), 7.95 (d, J=6.2 Hz, 1H), 7.50 (d, J=6.2 Hz, 1H), 6.69-6.54 (m, 2H), 6.13-5.92 (m, 2H), 5.92-5.76 (m, 1H), 5.45-5.15 (m, 4H), 4.52-4.44 (m, 2H), 4.16-4.06 (m, 3H), 3.76-3.66 (m, 1H), 3.49-3.41 (m, 1H), 3.11-2.97 (m, 1H), 2.83-2.76 (m, 2H), 2.55-2.43 (m, 2H); LCMS (ESI) m/z calc. for C₂₆H₂₇FN₄O₃ 462.21; found, 463.2 [M+H]⁺.

### Preparation 26. 1³-((3-Fluoro-2-methoxyphenyl)amino)-1⁴,1⁵,1⁶,1⁷-tetrahydro-1 1H-3-oxa-1(2,7)-pyrrolo[3,2-c]pyridina-2(4,3)-pyridinacyclooctaphan-6-en-1⁴-one (P26)

A mixture of P25 (300 mg, 0.649 mmol) in anh. DCM (65 mL) was degassed by argon for 10 min.

To the reaction mixture was added Grubbs catalyst 2nd generation (83 mg, 0.097 mmol) and it was stirred at 50° C. for 1.5 h. The reaction mixture was concentrated in vacuo. The residue was purified by silica gel column chromatography (0-10% MeOH in DCM) to afford P26 (trans/cis isomers, 200 mg, 71%) as ayellow solid. ¹H NMR (400 MHz, CDCl₃), δ: 12.51-12.40 (br. s, 1H), 8.64-8.59 (s, 1H), 8.09-8.00 (m, 1H), 7.62 (br. s, 1H), 7.26-7.19 (m, 1H), 6.89-6.65 (m, 1H), 6.65-6.54 (m, 1H), 6.22-5.86 (m, 3H), 5.61 (br. s, 1H), 5.01-4.89 (m, 1H), 4.89-4.83 (m, 1H), 4.34-4.01 (m, 4H), 3.63-3.32 (m, 3H), 3.04-2.86 (m, 1H), 2.82-2.67 (m, 2H), 2.42-2.26 (m, 1H); LCMS (ESI) m/z calc. for C₂₄H2₃FN₄O₃ 434.18; found, 435.1 [M+H]⁺.

### Synthesis of tert-butyl 3-(3,3-dimethoxypropyl)-5-[(3-fluoro-2-methoxy-phenyl)carbamothioyl]-4-hydroxy-6-oxo-2,3-dihydropyridine-1-carboxylate (P33)

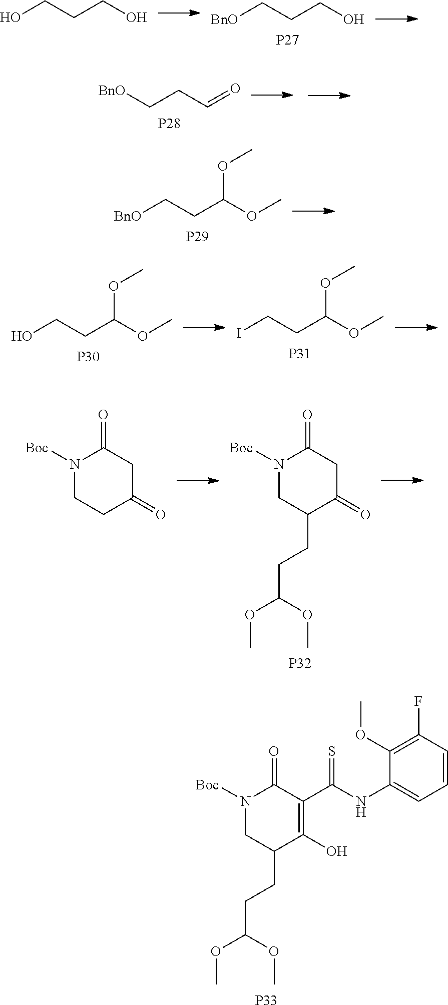

### Preparation 27. 3-Benzyloxypropan-1-ol (P27)

KOH (26.09 g, 464.9 mmol, 4.23 eq) and bromomethyl benzene (18.8 g, 109.9 mmol, 1 eq) were added in portions to propane-1,3-diol (37 g, 488.0 mmol, 4.44 eq). The mixture was stirred at 25° C. for 5 h. The reaction mixture was diluted with H₂O (50 mL) and extracted with EtOAc (2×30 mL). The combined organic layers were washed with brine (50 mL), dried (Na₂SO₄), filtered and concentrated under reduced pressure. The product P27 was purified by column chromatography, (silica gel, Hexane:EtOAc=5:1-1:1) to give 15.6 g of light-yellow oil (84%).

### Preparation 28. 3-Benzyloxypropanal (P28)

To a cooled (−78° C.) solution of oxalyl chloride (9.6 ml, 1,2 eq.) in DCM (260 ml) was added dry DMSO (16 ml, freshly distilled from CaH2, 2,4 eq) dropwise and the resulting mixture was stirred at −78° C. for 5 min. A solution of P27 (15,6 g, 1 eq) in DCM (60 ml) was then added dropwise and the mixture was stirred at −78° C. for 20 min. Triethylamine (78 ml, 6 eq.) was then introduced rapidly, after stirring for 10 min at −78° C., the reaction mixture was allowed to warm to rt and diluted with DCM (400 mL). The organic layer was washed with brine (2×100 mL). The combined organic extracts were dried over Na₂SO₄, filtered, and concentrated under reduced pressure. Purification by flash chromatography on silica gel (Hexane:EtOAc=10:1-9:1) to give 11.5 g of light-yellow oil (72%).

### Preparation 29. 3,3-Dimethoxypropoxymethylbenzene (P29)

A solution of P28 (11,5 g, 1 eq), trimethyl orthoformate (11.5 ml, 1,5 eq), and PTSA (650 mg, 0.05 eq) in MeOH (220 ml) was stirred at rt for 1 h. After addition of sat. NaHCO₃ solution (˜25 mL) and H₂O, the mixture was extracted with DCM (3×100 ml). The organic layer was dried over Na₂SO₄ and the solvent was evaporated under reduced pressure to give colorless oil (12,8 g, 88%).

### Preparation 30. 3,3-Dimethoxypropan-1-ol (P30)

To a solution of P29 (12.8 g, 1 eq) in MeOH (200 mL) was added 10% Pd/C (1.4 g, 0.02 eq) and the mixture was stirred at rt for 17 h under H₂ atmosphere. The reaction mixture was filtered through a pad of celite and the filtrate was rotary evaporated to dryness to give a colorless oil P30 (6.56 g, 89%).

### Preparation 31. 3-Iodo-1,1-dimethoxy-propane (P31)

To a solution of P30 (6.56 g, 1 eq) in DCM (200 mL) were added PPh₃ (15 g, 1.05 eq) and imidazole (4.65 g, 1.25 eq). To the reaction mixture was added portionwise iodine (15.3 g, 1.1 eq). The mixture was stirred at rt for 24 h. Reaction mixture was washed with 10% aq. sol. Na₂SO₃, brine, dried over Na₂SO₄ and rotary evaporated. To the residue was added hexane (100 ml), the mixture was stirred at rt for 5 min, then filtered. The solid was washed with hexane twice. Combined filtrate was rotary evaporated to dryness, giving P31 as light-yellow oil (8.92 g, 71%).

### Preparation 32. tert-Butyl 5-(3,3-dimethoxypropyl)-2,4-dioxo-piperidine-1-carboxylate (P32)

LiHMDS (1 M in THF, 7.5 mL) was added dropwise to a stirred solution of tert-butyl 2,4-dioxopiperidine-1-carboxylate (5.5 g, 1 eq) and P31 (8.92 g, 1.45 eq) in anh. THF (100 mL) at-50° C. under nitrogen. The mixture was allowed to warm slowly to rt, diluted with DCM (500 mL) and washed with KHSO₄ (5%, 800 ml). The organic phase was dried (Na₂SO₄), filtered and concentrated under reduced pressure. The product was purified by column chromatography (silica gel, CHCl₃-MeOH 0-2%) to give P32 as a yellow oil (7.32 g, 90%).

### Preparation 33. tert-Butyl 3-(3,3-dimethoxypropyl)-5-[(3-fluoro-2-methoxy-phenyl)carbamothioyl]-4-hydroxy-6-oxo-2,3-dihydropyridine-1-carboxylate (P33)

To a solution of P32 (7.62 g, I eq) in 150 ml DMF cooled to 0° C. was added 4.47 g (1.05 eq) of corresponding iso-thioisocyanate and 7.3 ml (2.25 eq) of Et3N. The reaction mixture was stirred for 24 h at rt. The reaction mixture was evaporated. The product was purified by column chromatography (silica gel, CCl₄—CHCl₃ 0-100%) to give P33 as a yellow oil (8.62 g, 74%).

### Synthesis of tert-butyl 5-(4,4-dimethoxybutyl)-3-[(3-fluoro-2-methoxy-phenyl)carbamoyl]-2,4-dioxo-piperidine-1-carboxylate (P40)

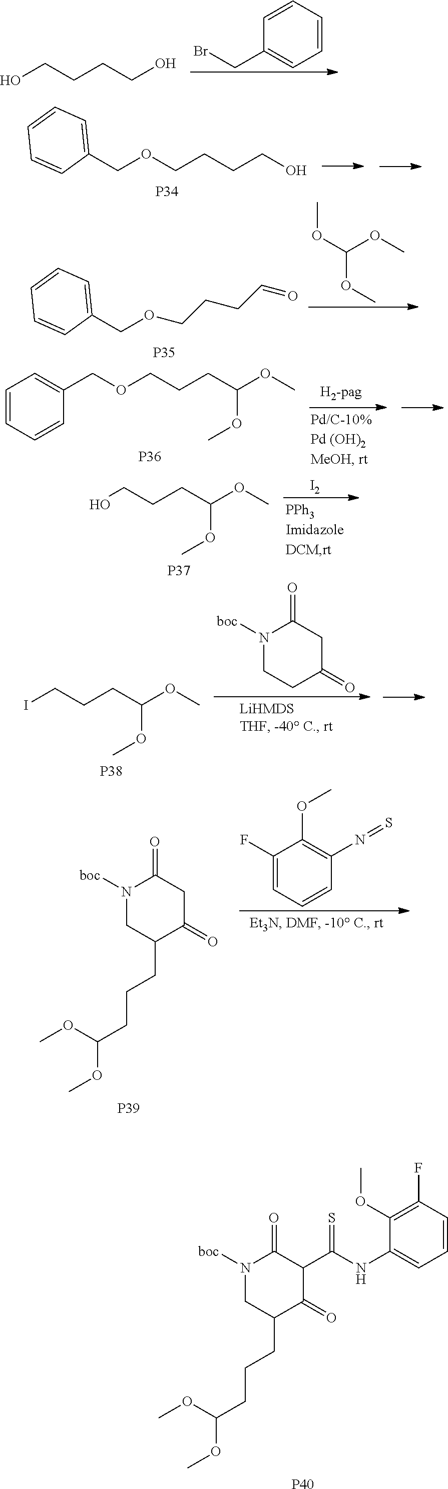

### Preparation 34. 4-Benzyloxybutan-1-ol (P34)

Butan-1,4-diol (64.80 g, 0.72 mol) was stirred at rt to which powdered KOH (38.44 g, 0.68 mol) and benzyl bromide (27.7 g, 0.16 mol) were added in equal portions over 1 h. After 3 h starring at rt, 50 mL of H₂O was added and reaction extracted thrice with diethyl ether (50 mL). The combined organic layers were dead over MgSO₄, and the solvent removed in vacuo. Column chromatography (4:1, Et₂O/Hexane) yielded 29.0 g, (99%) of P34 as a clear oil.

### Preparation 35. 4-Benzyloxybutanal (P35)

To a solution of oxalyl chloride (27.40 g, 0.21 mol) in 250 ml of DCM, pre-cooled to −70° C., was added over 35 min a solution of DMSO (29.90 g, 0.37 mol) in 50 ml of DCM. The solution was stirred for 10 min at −70° C. Then, a solution of P34 (30.00 g, 0.16 mol) in 150 ml of DCM was added over 35 min, the temperature being maintained at −65° to −70° C. Stirring was continued for another 20 min, and then, triethylamine (84.20 g, 0.80 mol) was added over 10 min at −60° to −70° C. The mixture was stirred for 20 min at −70° C., and then allowed to warm to 20° C. in 45 min. After the addition of 250 ml of water, stirring was continued for 15 min. The layers were separated and the aq. phase was extracted with 300 ml of DCM. The organic phase was washed with 300 ml of water and dried over sodium sulfate, and the solvent was evaporated in vacuo. Column chromatography (4:1, Hexane/ethyl acetate) yielded 28.8 g (97%) of P35 as a colorless oil.

### Preparation 36. 4,4-Dimethoxybutoxymethylbenzene (P36)

To a stirred solution of P35 (2.88 g, 0.016 mol) in methanol (20 ml) were added trimethyl orthoformate (5.14 g, 0.048 mol) followed by PTSA (5.6 mg, 2 mol %) at rt. The reaction mixture was stirred for 8 h at rt. The resulting mixture was washed with 5% sodium hydroxide aq. solution (5 mL) and extracted with DCM (2×30 mL). The organic layer was separated, dried (Na₂SO₄) and evaporated to give the crude compounds P36, which were used (after identification with 1H NMR), without further purification, in the iodination reaction in the next step. The yield of P36 was 2.60 g (74%) as a brown oil.

### Preparation 37. 4,4-Dimethoxybutan-1-ol (P37)

Pd/C-10%(1.4 g) and Pd(OH)₂/C (0.7 g) were added to a solution of P36 (13.9 g, 0.062 mol) in MeOH (200 ml) and reaction mixture was stirred at rt for 17 h under H₂ atmosphere. Control by TLC. Reaction mixture was filtered through a pad of celite and the filtrate was rotary evaporated to dryness to give pure P37. The yield of P37 was 8.25 g, 99%.

### Preparation 38. 4-Iodo-1,1-dimethoxy-butane (P38)

To a solution of P37 (8.25 g, 0.062 mol) in DCM (200 ml) were added PPh₃ (16.8 g, 0.064 mol) and Imidazole (5.2 g, 0.076 mol). Iodine (17.0 g, 0.067 mol) was added portionwise to the reaction mixture and the mixture was stirred at rt for 24 h. Reaction mixture was washed with 10% aq. sol. Na₂SO₃ (200 ml), brine, dried over Na₂SO₄ and rotary evaporated. To the residue was added hexane (200 ml), the mixture was stirred at rt for 5 min, then filtered. The solid was washed with hexane. Combined filtrate was rotary evaporated to dryness and used in the next step without puriication. The yield of P38 was 11 g, 74%.

### Preparation 39. tert-Butyl 5-(4,4-dimethoxybutyl)-2,4-dioxo-piperidine-1-carboxylate (P39)

To a solution of tert-butyl 2,4-dioxopiperidine-1-carboxylate (6.8 g, 0.032 mol) and P38 (11 g 0.045 mol) in THF (130 ml) at −40° C. was added LiHMDS (13.4 g, 0.08 mol) in THF (80 ml) dropwise, with the internal temperature of the reaction kept at −50-40° C. Reaction mixture was slowly wormed to ambient temperature and stirred for 17 h. To a reaction mixture was added water (200 ml) and ether (100 ml) and mixture was stirred at rt for 10 min. Aq. layer was separated, mixed with ether (100 ml) and EtOAc (100 ml) and acidified with 2N HCl until pH˜3-4. Organic layer was separated; aq. layer was extracted with ether. Combined organic extract was washed with brine, dried over Na₂SO₄ and rotary evaporated to dryness. The product was purified by column chromatography (silica gel, Hexane:EtOAc=3:1-1:1-0:1). The yield of P39 was 2.54 g, 24%.

### Preparation 40. tert-Butyl 5-(4,4-dimethoxybutyl)-3-[(3-fluoro-2-methoxy-phenyl)carbamothioyl]-2,4-dioxo-piperidine-1-carboxylate (P40)

To a solution of P39 (2.54 g, 0.0077 mol) and Et₃N (2.7 ml, 0.019 mol) in DMF (8 ml) at −10° C. was added 1-fluoro-3-isothiocyanato-2-methoxy-benzene (1.48 g, 0.0081 mol) dropwise and reaction mixture was stirred for 17 h at rt. To the reaction mixture was added water (20 ml) and product was extracted with ether. Combined organic extract was washed with brine, dried over Na₂SO₄ and rotary evaporated to dryness. The product was purified by column chromatography (silica gel, Hexane:EtOAc=3:1-1:1-0:1). The yield of P40 was 1.6 g, 60%.

### Synthesis of tert-Butyl 3-({[4-(aminomethyl)pyridin-3-yl]oxy}methyl)pyrrolidine-1-carboxylate (P42a)

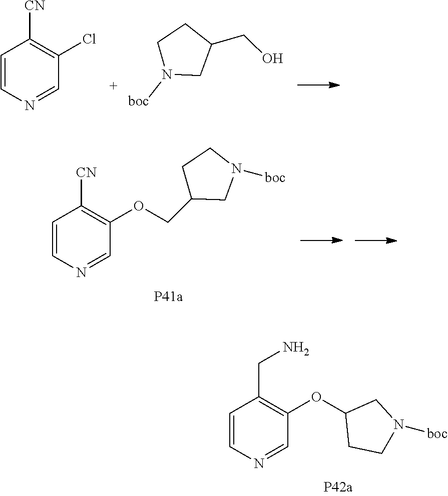

### Preparation 41. tert-Butyl 3-{[(4-cyanopyridin-3-yl)oxy]methyl}pyrrolidine-1-carboxylate (P41a)

To a stirred solution of tert-butyl 3-(hydroxymethyl)pyrrolidine-1-carboxylate (680 mg, 3.4 mmol) in DMF (10 mL) was added NaH (0.2 g, 5.0 mmol, 60%) and the resulting mixture was stirred at ambient temperature for 15 min. Then 3-chloro-4-cyanopyridine (470 mg, 3.4 mmol) was added, the solution was stirred for 1 h at rt. The reaction mixture was diluted with water and Et₂O. The organic layer was separated, washed with brine, dried over sodium sulfate, filtered, and the filtrate was evaporated. The residue after evaporation was subjected to column chromatography on silica gel eluting with DCM/EtOAc (0-10%) to give tert-butyl 3-{[(4-cyanopyridin-3-yl)oxy]methyl}pyrrolidine-1-carboxylate P41a (1.0 g, 99% yield). LCMS (ESI⁺) m z 304 [M+H].

### Preparation 42. tert-Butyl 3-({[4-(aminomethyl)pyridin-3-yl]oxy}methyl)pyrrolidine-1-carboxylate (P42a)

tert-Butyl 3-{[(4-cyanopyridin-3-yl)oxy]methyl}pyrrolidine-1-carboxylate P41a (1.0 g, 3.3 mmol) was dissolved in ethanol/AcOH (9:1, 10 mL). Pd/C (200 mg) was added and the mixture was stirred under H₂ atmosphere for 16 h. TLC indicated completion of the reaction. The catalyst was removed by filtration and the solution was evaporated to dryness. The residue was diluted sat. aq. solution of sodium bicarbonate and DCM. The organic layer was separated, washed with brine, dried over sodium sulfate, filtered, and the filtrate was evaporated. The residue after evaporation was subjected to column chromatography on silica gel eluting with DCM/MeOH (0-30%) to give P42a (690 mg, 69%). LCMS (ESI⁺) m/z 308 [M+H].

Using the procedures described above (Preparation 41 and Preparation 42)amines 42b, 42c, 42d, 42e, 42f, and 42g were prepared. Analytical data for the amines 42a, 42b, 42c, 42d, 42e, 42f, and 42g is presented in the Table 5.

TABLE 5
LCMS for the compounds 42(a-g)

| Com- | | | |
|---|---|---|---|
| pound | | [MH]⁺ | [MH]⁺ |
| # | Structure | calculated | found |
| 42a | 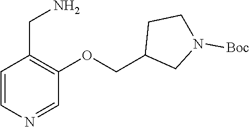 [SMILES: CC(C)(C)OC(=O)N1CCC(COc2cnccc2CN)C1] | 308.197 | 308 |
| 42b | 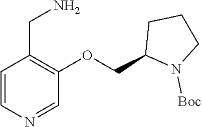 [SMILES: CC(C)(C)OC(=O)N1CCC[C@@H]1COc1cnccc1CN] | 308.197 | 308 |
| 42c | 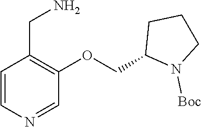 [SMILES: CC(C)(C)OC(=O)N1CCC[C@H]1COc1cnccc1CN] | 308.197 | 308 |
| 42d | 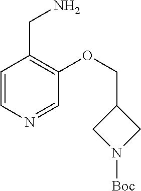 [SMILES: CC(C)(C)OC(=O)N1CC(COc2cnccc2CN)C1] | 294.182 | 294 |
| 42e | 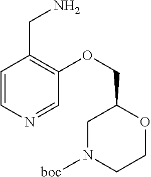 | 326.199 | 326 |
| 42f | 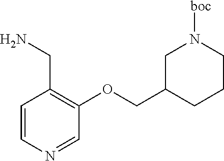 | 322.213 | 322 |
| 42g | 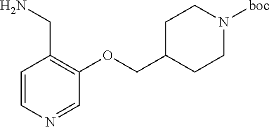 | 322.213 | 322 |

### Synthesis of 3-[2-[3-(3-aminopropoxy)-4-pyridyl]-3-(3-fluoro-2-methoxy-anilino)-4-oxo-1,5,6,7-tetrahydropyrrolo[3,2-c]pyridin-7-yl]propanal (P46)

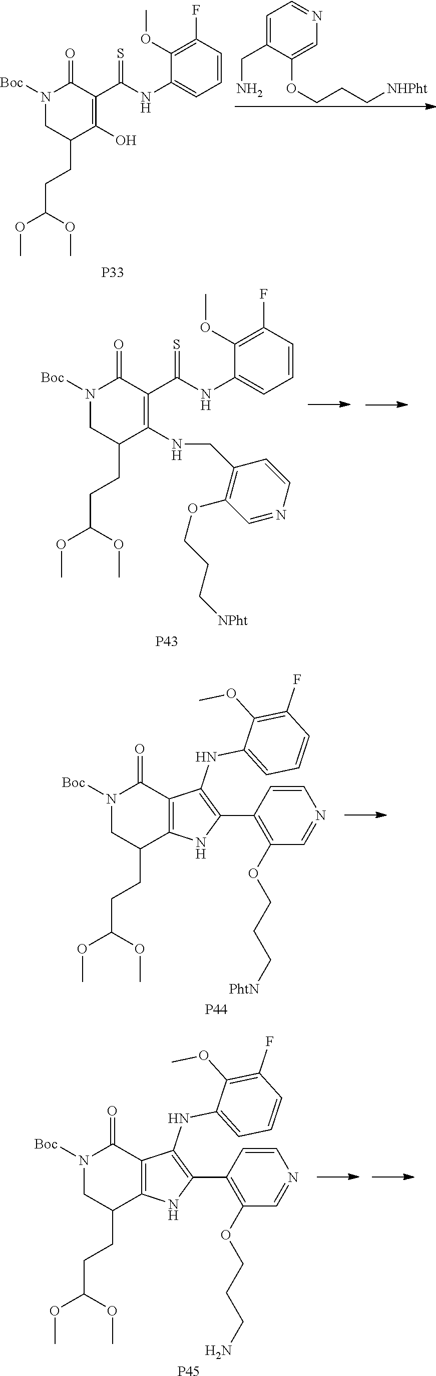

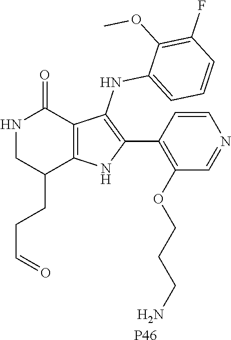 [SMILES: "P46" ; COc1c(F)cccc1Nc1c(-c2ccncc2OCCCN)[nH]c2c1C(=O)NCC2CCC=O]

### Preparation 43. tert-Butyl 3-(3,3-dimethoxypropyl)-4-[[3-[3-(1,3-dioxoisoindolin-2-yl)propoxy]-4-pyridyl]methylamino]-5-[(3-fluoro-2-methoxy-phenyl)carbamothioyl]-6-oxo-2,3-dihydropyridine-1-carboxylate (P43)

2-[3-[[4-(aminomethyl)-3-pyridyl]oxy]propyl]isoindoline-1,3-dione (1,1 g, 1,1 eq) and DIPEA (1,6 g, 3 eq) were added to a stirred and cooled to 0° C. solution of thioamide P33 (1,02 g, 1 eq) in dry acetonitrile (15 mL) followed by portion wise addition of BSTFA (1,6 g, 3 eq). The reaction mixture was sealed and stirred at 80° C. until full thioamide P33 consumption (18 h, LCMS monitoring) and concentrated under reduced pressure. The residue was purified by column chromatography (silica gel, DCM-EtOH gradient (0-5%), giving P43 as a brown oil (1,18 g, 76%).

### Preparation 44. tert-butyl 7-(3,3-dimethoxypropyl)-2-[3-[3-(1,3-dioxoisoindolin-2-yl)propoxy]-4-pyridyl]-3-(3-fluoro-2-methoxy-anilino)-4-oxo-6,7-dihydro-1H-pyrrolo[3,2-c]pyridine-5-carboxylate (P44)

To a solution of 1,18 g (1 eq.) of P43 in MeOH (30 mL) was added 171 mg TFA (1 eq) and 1.1 mL (5 eq) H₂O₂(30% aq. solution). The mixture was stirred at 60° C. until full conversion to P44 (3 h, LCMS monitoring), additional H₂O₂ could be used if needed. The mixture was cooled to rt, treated with 25 ml of a saturated Na₂SO₃ solution. Products were extracted with ethyl acetate (3×20 ml). The organic layer was dried over Na₂SO₄, filtered, and evaporated on a rotary evaporator. The residue was purified by column chromatography (silica gel, DCM/EtOH gradient (0-5%)) giving compound P44 as a yellow solid (680 rng, 60%).

### Preparation 45. tert-butyl 2-[3-(3-aminopropoxy)-4-pyridyl]-7-(3,3-dimethoxypropyl)-3-(3-fluoro-2-methoxy-anilino)-4-oxo-6,7-dihydro-1H-pyrrolo[3,2-c]pyridine-5-carboxylate (P45)

To a solution of 660 mg (1 eq) of P44 in EtOH was added 170 μl (4 eq) N₂H₄×H₂O and the mixture was stirred at reflux for 2 h. The reaction mixture was rotary evaporated to dryness, to the residue was added Et₂O (50 ml), mixture was stirred for 10 min then filtered, the solid was washed with Et₂O twice. Filtrate was rotary evaporated to dryness, giving P45 as a yellow solid (510 mg, 93%).

### Preparation 46. 3-[2-[3-(3-aminopropoxy)-4-pyridyl]-3-(3-fluoro-2-methoxy-anilino)-4-oxo-1,5,6,7-tetrahydropyrrolo[3,2-c]pyridin-7-yl]propanal (P46)

To a solution of 110 mg (1 eq) of P45 in 1,4-dioxane was added 45 mg (1,5 eq) of PTSA×H₂0. The mixture was stirred at 55° C. until full deprotection of aldehyde (2h, LCMS monitoring, Boc group might stay). The reaction mixture was cooled to rt, diluted with DCM and treated with sat. NaHCO₃, brine, combined extracts were dried over Na₂SO₄ and rotary evaporated to dryness. The residue was used in next step without additional purification.

### Synthesis of 3-{3-[(3-fluoro-2-methoxyphenyl)amino]-4-oxo-2-[3-(piperidin-3-ylmethoxy)pyridin-4-yl]-4,5,6,7-tetrahydro-1H-pyrrolo[3,2-c]pyridin-7-yl}propanal (P49)

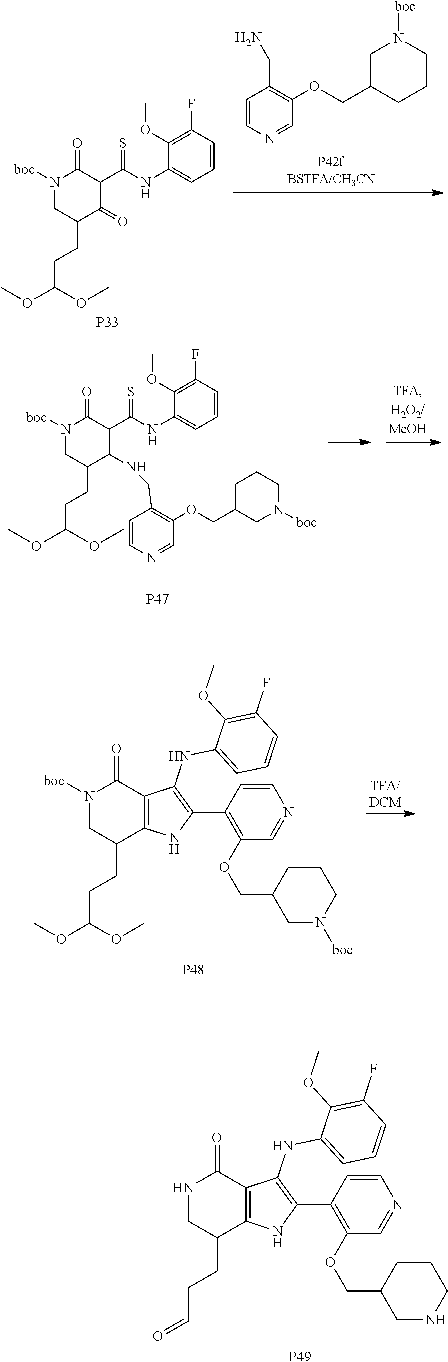

### Preparation 47. tert-Butyl 4-{[(3-{[1-(tert-butoxycarbonyl)piperidin-3-yl]methoxy}pyridin-4-yl)methyl]amino}-5-(3,3-dimethoxypropyl)-3-{[(3-fluoro-2-methoxyphenyl)amino]carbonothioyl}-2-oxopiperidine-1-carboxylate (P47)

To a solution of tert-butyl 5-(3,3-dimethoxypropyl)-3-{[(3-fluoro-2-methoxyphenyl)amino]carbonothioyl}-2,4-dioxopiperidine-1-carboxylate P33 (500 mg, 1.0 mmol, 1.0 eq), tert-butyl 3-({[4-(aminomethyl)pyridin-3-yl]oxy}methyl)piperidine-1-carboxylate P42f (483 mg, 1.5 mmol, 1.5 eq) in dry acetonitrile (10 mL) N,O-bis(trimethylsilyl)trifluoroacetamide (BSTFA, 0.8 mL, 3.0 mmol, 3.0 eq) was added dropwise at 0° C., and then the reaction mixture was stirred at 80° C. for 16 h. The solution was concentrated, the residue was purified by silica gel column chromatography (DCM:MeOH 0-2%) to give P47 (430 mg, 53%) as a yellow oil. ¹H NMR (400 MHz, DMSO-d₆), δ: 13.52 (s, 1H), 13.26 (s, 1H), 8.39 (s, 1H), 8.24 (d, J=4.6 Hz, 1H), 7.56 (s, 1H), 7.37 (d, J=4.4 Hz, 1H), 7.21-6.96 (m, 2H), 4.69 (s, 2H), 4.33 (s, 1H), 4.03 (d, J=14.0 Hz, 4H), 3.85-3.66 (m, 4H), 3.47 (d, J=15.0 Hz, 1H), 3.19 (d, J=10.4 Hz, 6H), 3.06 (s, 1H), 2.84 (s, 2H), 2.50 (s, 3H), 1.89 (d, J=40.6 Hz, 2H), 1.64 (d, J=6.0 Hz, 4H), 1.46 (s, 9H), 1.35 (s, 9H). LCMS (ESI⁺) m/z 802 [M+H].

### Preparation 48. tert-Butyl 2-(3-{[1-(tert-butoxycarbonyl)piperidin-3-yl]methoxy}pyridin-4-yl)-7-(3,3-dimethoxypropyl)-3-[(3-fluoro-2-methoxyphenyl)amino]-4-oxo-1,4,6,7-tetrahydro-5H-pyrrolo[3,2-c]pyridine-5-carboxylate (P48)

To a solution of P47 (430 mg, 0.5 mmol, 1.0 eq) in methanol (20 mL)trifluoroacetic acid (0.04 mL, 0.5 mmol, 1.0 eq) and 35% w/w hydrogen peroxide (0.10 mL, 1.0 mmol, 1.0 eq) were added dropwise at 0° C. The reaction mixture was stirred at 50° C. for 1 h, concentrated and treated with aq. saturated solution of sodium bicarbonate to adjust pH=7. The mixture was extracted with DCM three times. The combined organic layers were dried over Na₂SO₄, filtered, and concentrated to give P48 (400 mg) as a yellow foam. The compound was used for the next step without additional purification. LCMS (ESI⁺) m/z 768 [M+H]⁺.

### Preparation 49. 3-{3-[(3-Fluoro-2-methoxyphenyl)amino]-4-oxo-2-[3-(piperidin-3-ylmethoxy)pyridin-4-yl]-4,5,6,7-tetrahydro-1H-pyrrolo[3,2-c]pyridin-7-yl}propanal (P49)

To a solution of P48 (400 mg, 0.5 mmol, 1.0 eq) in DCM (10.0 mL)trifluoroacetic acid (2.0 mL, 26 mmol, 50 eq) was added dropwise. The reaction mixture was stirred at rt for 3 h. The solution was treated with a saturated aq. solution of sodium bicarbonate until pH=7 and extracted with DCM/t-BuOH (10 mL, 1:1) three times. The combined organic layers were dried over Na₂SO₄, filtered and concentrated in vacuo to give P49 (250 mg, crude product). LCMS (ESI⁺) m/z 522 [M+H]⁺.

### Synthesis of 7-allyl-3-[(3-fluoro-2-methoxyphenyl)amino]-2-{3-[(1-hex-5-enoylpiperidin-3-yl)methoxy]pyridin-4-yl}-1,5,6,7-tetrahydro-4H-pyrrolo[3,2-c]pyridin-4-one (P53)

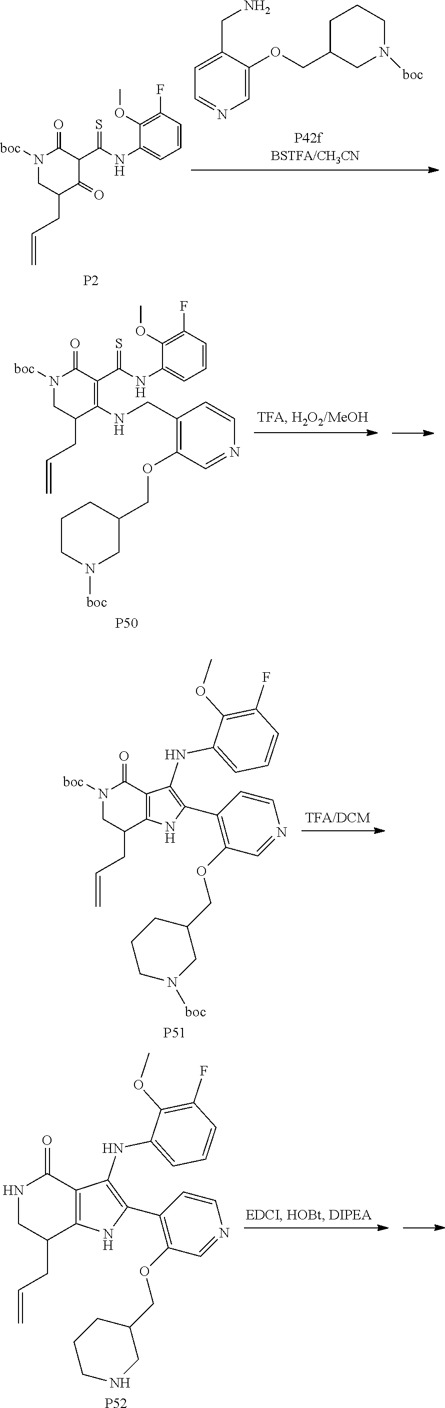

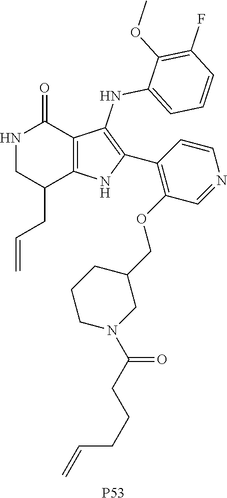 [SMILES: "P53" ; C=CCCCC(=O)N1CCCC(COc2cnccc2-c2[nH]c3c(c2Nc2cccc(F)c2OC)C(=O)NCC3CC=C)C1]

### Preparation 50. tert-Butyl 3-allyl-4-{[(3-{[1-(tert-butoxycarbonyl)piperidin-3-yl]methoxy}pyridin-4-yl)methyl]amino}-5-{[(3-fluoro-2-methoxyphenyl)amino]carbonothioyl}-6-oxo-3,6-dihydropyridine-1(2H)-carboxylate (P50)

To a solution of tert-butyl 5-allyl-3-(3-fluoro-2-methoxyphenylcarbamothioyl)-4-hydroxy-2-oxo-5,6-dihydropyridine-1(2H)-carboxylate P2 (1.0 g, 2.3 mmol, 1.0 eq), tert-butyl 3-({[4-(aminomethyl)pyridin-3-yl]oxy}methyl)piperidine-1-carboxylate P42f (0.88 g, 2.7 mmol, 1.2 eq) and molecular sieves (4 A, 1 g) in dry acetonitrile (10 mL) N,O-bis(trimethylsilyl)trifluoroacetamide (BSTFA, 1.8 mL, 6.9 mmol, 3.0 eq) was added dropwise at 0° C., and then the reaction mixture was stirred at 80° C. for 16 h. The solid was removed by filtration and the solution was concentrated, treated with water, and extracted with CH₂C₁₂ three times. The combined organic layers were dried over Na₂SO₄, filtered, and concentrated in vacuo. The residue was puriied by silica gel column chromatography (DCM:EtOAc 0-50%) to give P50 (1.47 g, 87%) as a yellow oil. LCMS (ESI⁺) m/z 740 [M+H].

### Preparation 51. tert-Butyl 7-allyl-2-(3-{[1-(tert-butoxycarbonyl)piperidin-3-yl]methoxy}pyridin-4-yl)-3-[(3-fluoro-2-methoxyphenyl)amino]-4-oxo-1,4,6,7-tetrahydro-5H-pyrrolo[3,2-c]pyridine-5-carboxylate (P51)

To a solution of P50 (1.47 g, 1.98 mmol, 1.0 eq) in methanol (40 mL) TFA (0.15 mL, 1.98 mmol, 1.0 eq) and 35% w/w hydrogen peroxide (0.38 mL, 3.97 mmol, 2.0 eq) were added dropwise at 0° C. The reaction mixture was stirred at 50° C. for 1 h, concentrated and treated with aq. sat. solution of sodium bicarbonate to adjust pH=7. The mixture was extracted with DCM three times.

The combined organic layers were dried over Na₂SO₄>, filtered, and concentrated in vacuo to give P51 (900 mg, 64%) as a yellow foam. ¹H NMR (400 MHz, DMSO-d₆), δ: 11.95 (s, 1H), 11.44 (s, 1H), 8.41 (s, 1H), 8.11 (s, 1H), 7.28 (t, J=5.1 Hz, 1H), 7.19 (d, J=8.6 Hz, 1H), 6.59 (s, 1H), 6.47-6.34 (m, 1H), 6.03 (t, J=8.0 Hz, 1H), 5.84 (d, J=6.9 Hz, 1H), 5.18-5.00 (m, 2H), 3.96-3.92 (m, 5H), 3.85 (s, 3H), 3.76 (d, J=19.2 Hz, 2H), 3.17 (s, 1H), 2.90-2.63 (m, 2H), 2.56 (s, 1H), 2.28 (s, 1H), 1.79 (s, 1H), 1.61 (s, 1H), 1.46-1.39 (m, 9H), 1.34 (s, 13H). LCMS (ESI⁺) m/z 706 [M+H]⁺.

### Preparation 52. 7-Allyl-3-[(3-fluoro-2-methoxyphenyl)amino]-2-[3-(piperidin-3-ylmethoxy)pyridin-4-yl]-1,5,6,7-tetrahydro-4H-pyrrolo[3,2-c]pyridin-4-one (P52)

To a solution of P51 (900 mg, 1.27 mmol, 1.0 eq) in DCM (10.0 mL) TFA (4.5 mL) was added dropwise. The reaction mixture was stirred at rt for 3 h. The solution was treated with a sat. aq. solution of sodium bicarbonate until pH=7 and extracted with DCM three times. The combined organic layers were dried over Na₂SO₄, filtered and concentrated in vacuo to give P52(500 mg, 78% yield) as an orange solid. ¹H NMR (400 MHz, DMSO-d₆), δ: 11.36-11.32 (m, 1H), 8.74 (s, 1H), 8.52 (d, J=4.1 Hz, 2H), 8.28 (d, J=5.7 Hz, 1H), 7.82 (s, 1H), 7.54 (d, J=3.3 Hz, 1H), 7.24 (s, 1H), 6.65-6.61 (m, 1H), 6.58-6.46 (m, 1H), 5.98-5.75 (m, 2H), 5.21-5.08 (m, 3H), 4.18-3.98 (m, 3H), 3.50-3.41 (m, 1H), 3.26-3.22 (m, 4H), 2.84-2.65 (m, 2H), 2.57-2.53 (m, 1H), 2.34 (s, 2H), 1.85-1.82 (m, 2H), 1.65-1.60 (m, 1H), 1.42-1.19 (m, 1H). LCMS (ESI⁺) m/z 506 [M+H]⁺.

### Preparation 53. 7-Allyl-3-[(3-fluoro-2-methoxyphenyl)amino]-2-{3-[(1-hex-5-enoylpiperidin-3-yl)methoxy]pyridin-4-yl}-1,5,6,7-tetrahydro-4H-pyrrolo[3,2-c]pyridin-4-one (P53)

To solution of P52 (150 mg, 0.3 mmol, 1 eq), DIPEA (0.08 mL, 0.45 mmol, 1.5 eq), hex-5-enoic acid (37 mg, 0.33 mmol, 1.1 eq), HOBt (40 mg. 0.3 mmol, 1 eq) in DCM (2 mL) EDAC (75 mg, 0.39 mmol) was added. Then the reaction mixture was stirred at rt for overnight. Then the reaction mixture was treated with water, and extracted with DCM three times. The combined organic layers were dried over Na₂SO₄, filtered, and concentrated in vacuo. The residue was purified by silica gel column chromatography (EtOAc:MeOH 0-10%) to give P53 (125 mg, 87%) as a yellow oil. ¹H NMR (400 MHz, DMSO-d₆), δ: 11.94 (s, 1H), 11.15 (d, J=9.7 Hz, 2H), 8.39 (d, J=7.0 Hz, 2H), 8.09 (d, J=4.8 Hz, 1H), 8.02 (d, J=2.9 Hz, 1H), 7.47 (d, J=11.6 Hz, 1H), 7.41 (s, 1H), 7.33-7.19 (m, 2H), 7.05 (s, 2H), 6.66-6.52 (m, 2H), 6.47-6.35 (m, 2H), 6.04-5.93 (m, 2H), 5.78-5.75 (in, 4H), 5.17-5.03 (m, 4H), 4.93-4.88 (m, 4H), 4.21-3.91 (m, 7H), 3.85 (t, J=13.9 Hz, 7H), 3.57 (s, 1H), 3.48-3.44 (m, 2H), 3.23-2.94 (m, 6H), 2.41-2.21 (m, 5H), 2.21-2.08 (m, 2H), 2.02-1.98 (m, 4H), 1.89-1.77 (m, 4H), 1.72-1.52 (m, 5H), 1.50-1.39 (m, 4H), 1.30-1.23 (m, 3H). LCMS (ESI⁺) m/z 602 [M+H]⁺.

### Synthesis of 4-[3-(3-fluoro-2-methoxy-anilino)-4-oxo-2-[3-(4-piperidylmethoxy)-4-pyridyl]-1,5,6,7-tetrahydropyrrolo[3,2-c]pyridin-7-yl]butanal (P56)

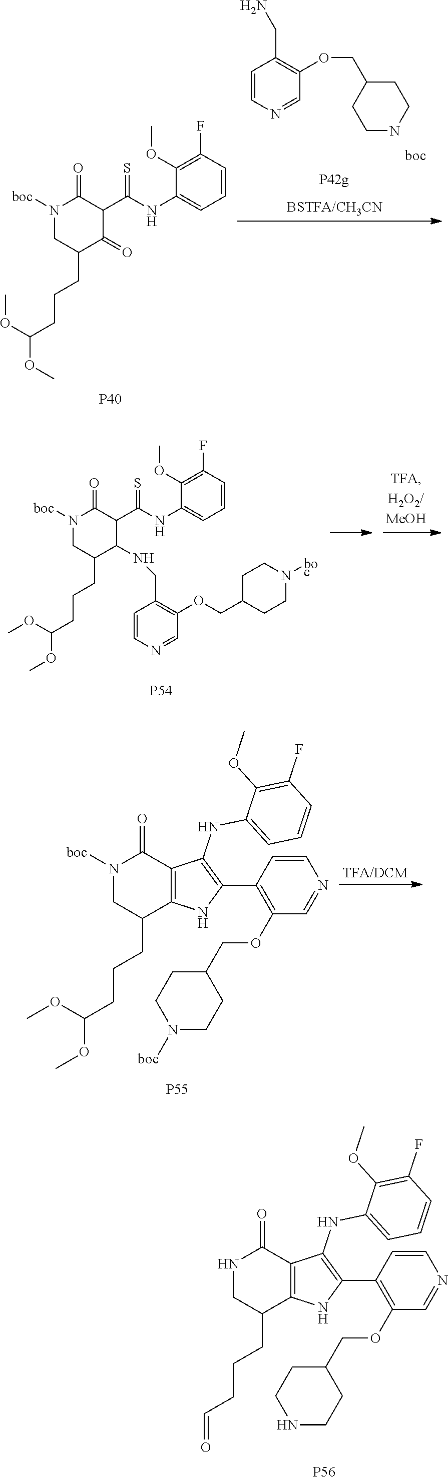

### Preparation 54. tert-butyl 4-{[(3-{[1-(tert-butoxycarbonyl)piperidin-4-yl]methoxy}pyridin-4-yl)methyl]amino}-5-(4,4-dimethoxybutyl)-3-{[(3-fluoro-2-methoxyphenyl)amino]carbonothioyl}-2-oxopiperidine-1-carboxylate (P54)

To a solution of P40 (500 mg, 0.97 mmol, 1.0 eq), P42g (470 mg, 1.5 mmol, 1.5 eq) in dry acetonitrile (10 mL) N,O-bis(trimethylsilyl)trifluoroacetamide (BSTFA, 0.8 mL, 3.0 mmol, 3.0 eq) was added dropwise at 0° C., and then the reaction mixture was stirred at 80° C. for 16 h. The solution was concentrated, the residue was purified by silica gel column chromatography (DCM:MeOH 0-2%) to give P54 (400 mg, 40%) as ayellow oil. LCMS (ESI⁺) m/z 817 [M+H]⁺.

### Preparation 55. tert-butyl 2-(3-{[1-(tert-butoxycarbonyl)piperidin-4-yl]methoxy}pyridin-4-yl)-7-(4,4-dimethoxybutyl)-3-[(3-fluoro-2-methoxyphenyl)amino]-4-oxo-1,4,6,7-tetrahydro-5H-pyrrolo[3,2-c]pyridine-5-carboxylate (P55)

To a solution of give P54 (400 mg, 0.5 mmol, 1.0 eq) in methanol (20 mL)trifluoroacetic acid (0.04 mL, 0.5 mmol, 1.0 eq) and 35% w/w hydrogen peroxide (0.1 mL, 0.98 mmol, 2.0 eq) were added dropwise at 0° C. The reaction mixture was stirred at 50° C. for 1 h, concentrated and treated with aq. sat. solution of sodium bicarbonate to adjust pH=7. The mixture was extracted with DCM three times. The combined organic layers were dried over Na₂SO₄, filtered, and concentrated to give P56 (350 mg) as a yellow foam. The compound was used for the next step without additional purification. LCMS (ESI⁺) m/z 782 [M+H]⁺.

### Preparation 56. 4-[3-(3-Fluoro-2-methoxy-anilino)-4-oxo-2-[3-(4-piperidylmethoxy)-4-pyridyl]-1,5,6,7-tetrahydropyrrolo[3,2-c]pyridin-7-yl]butanal (P56)

To a solution of P55 (350 mg, 0.45 mmol, 1.0 eq) in DCM (10.0 mL)trifluoroacetic acid (1.7 mL, 22 mmol, 50 eq) was added dropwise. The reaction mixture was stirred at rt for 3 h. The solution was treated with a saturated aqueous solution of sodium bicarbonate until pH=7 and extracted with DCM/t-BuOH (10 mL, 1:1) three times. The combined organic layers were dried over Na₂SO₄, filtered and concentrated in vacuo to give P56 (200 mg, crude product). LCMS (ESI⁺) m/z 536 [M+H].

### Synthesis of 2-(3-(3-fluoro-2-methoxyanilino)-4-oxo-2-3-[2-(2-piperazinoethoxy)ethoxy]-4-pyridyl-4,5,6,7-tetrahydro-1H-pyrrolo[3,2-c]pyridin-7-yl)acetic acid (P61)

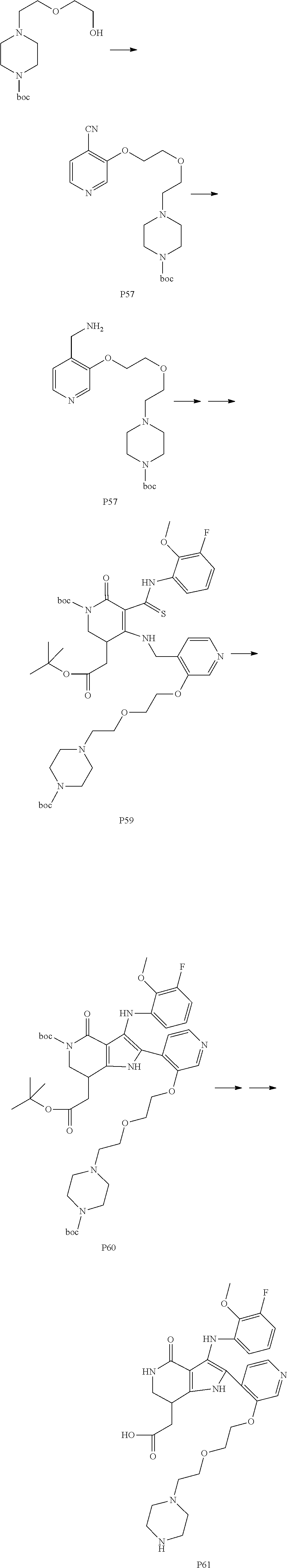

### Preparation 57. tert-Butyl 4-(2-2-[(4-cyano-3-pyridyl)oxy]ethoxyethyl)tetrahydro-1(2H)-pyrazinecarboxylate (P57)

tert-Butyl 4-[2-(2-hydroxyethoxy)ethyl]piperazine-1-carboxylate (3 g, 10.9 mmol) was added at rt to a suspension of 60%-NaH (0.523 g, 13.6 mmol) in 30 ml DMF, the solution was stirred for 1 h at rt. Next, 3-chloro-4-cyanopyridine (1.5 g, 10.9 mmol) in 10 mL of DMF was added to the solution, and the reaction mixture was stirred for 1 h at rt. The reaction mass was worked up by adding 30 ml of water. The reaction products were extracted with ethyl acetate (3×50 ml). The organic layer was dried over Na₂SO₄, evaporated on a rotary evaporator. The residue was purified by silica gel column chromatography (DCM:MeOH) to give P57 (2 g, 49%). LCMS (ESI⁺) m/z 377 [M+H]⁺.

### Preparation 58. tert-Butyl 4-[2-(2-[4-(aminomethyl)-3-pyridyl]oxyethoxy)ethyl]tetrahydro-1(2H)-pyrazinecarboxylate (P58)

In an autoclave, 1 g of 64%-Ni—R^{a}/SiO₂ and 10 ml of NH₃/H₂O were added to 2 g (5.3 mmol) of P57 in 50 ml of absolute MeOH. The reaction was stirred in an atmosphere of hydrogen under a pressure of 30 bar at rt for 24 h. After that, the precipitate was filtered off, washed with MeOH, the filtrate was evaporated. The residue was purified by silica gel column chromatography (DCM:MeOH) to give P58 (0.9 g, 47%). LCMS (ESI⁺) m/z 381 [M+H]⁺.

### Preparation 59. tert-Butyl 4-2-[2-(4-[(1-(tert-butoxycarbonyl)-3-[2-(tert-butoxy)-2-oxoethyl]-5-[(3-fluoro-2-methoxyanilino)carbothioyl]-6-oxo-1,2,3,6-tetrahydro-4-pyridinylamino)methyl]-3-pyridyloxy)ethoxy]ethyltetrahydro-1(2H)-pyrazinecarboxylate (P59)

To a solution of thioamide (500 mg, 1 mmol) in anh. acetonitrile (7 mL) at 0° C. was added P58 (0.55 g, 1.5 mmol) and N,O-bis(trimethylsilyl)trifluoroacetamide (BSTFA, 3 mmol). The mixture was stirred at 80° C. for overnight. The solution was concentrated to remove solvent. The crude was puriied by silica-gel column chromatography (DCM:MeOH) to give P59 (0.57 g, 67%), as a yellow oil. LCMS (ESI⁺) m/z 874 [M+H]⁺.

### Preparation 60. tert-Butyl 2-[3-(2-2-[4-(tert-butoxycarbonyl)piperazino]ethoxyethoxy)-4-pyridyl]-7-[2-(tert-butoxy)-2-oxoethyl]-3-(3-fluoro-2-methoxyanilino)-4-oxo-1,4,6,7-tetrahydro-5H-pyrrolo[3,2-c]pyridine-5-carboxylate (P60)

To a solution of P59 (0.6 g, 0.68 mmol) in methanol (12 mL) was added TFA (0.08 mL, 1 mmol) and hydrogen peroxide (30% in water, 0.2 mL, 5.44 mmol). The mixture was stirred at 50° C. for 2 h, then concentrated and treated with aq. saturated solution of sodium bicarbonate to adjust pH=7. The mixture was extracted with DCM three times. The combined organic layers were dried over Na₂SO₄(,), filtered, and concentrated in vacuo to give a residue. The crude was purified by silica-gel column chromatography (DCM:THF=2:1) to give P60 (290 mg, 50%). LCMS (ESI⁺) m z 839 [M+H].

### Preparation 61. 2-(3-(3-Fluoro-2-methoxyanilino)-4-oxo-2-3-[2-(2-piperazinoethoxy)ethoxy]-4-pyridyl-4,5,6,7-tetrahydro-1H-pyrrolo[3,2-c]pyridin-7-yl)acetic acid (P61)

To a solution of P60 (290 mg, 0.34 mmol, 1.0 eq) in dioxane (3 mL) a 3M solution of HCl in dioxane was added dropwise at 0° C. The reaction mixture was stirred at rt for 3 h. The solid was filtrated, washed with dioxane two times and dried to give hydrochloride of amino acid P61 (250 mg, 99%) as an orange solid. LCMS (ESI⁺) m/z 583 [M+H]⁺.

### Synthesis of 4-(3-[(3-fluoro-2-methoxyphenyl)amino]-2-{3-[(2S)-morpholin-2-ylmethoxy]pyridin-4-yl}-4-oxo-4,5,6,7-tetrahydro-1H-pyrrolo[3,2-c]pyridin-7-yl)butanal (P64)

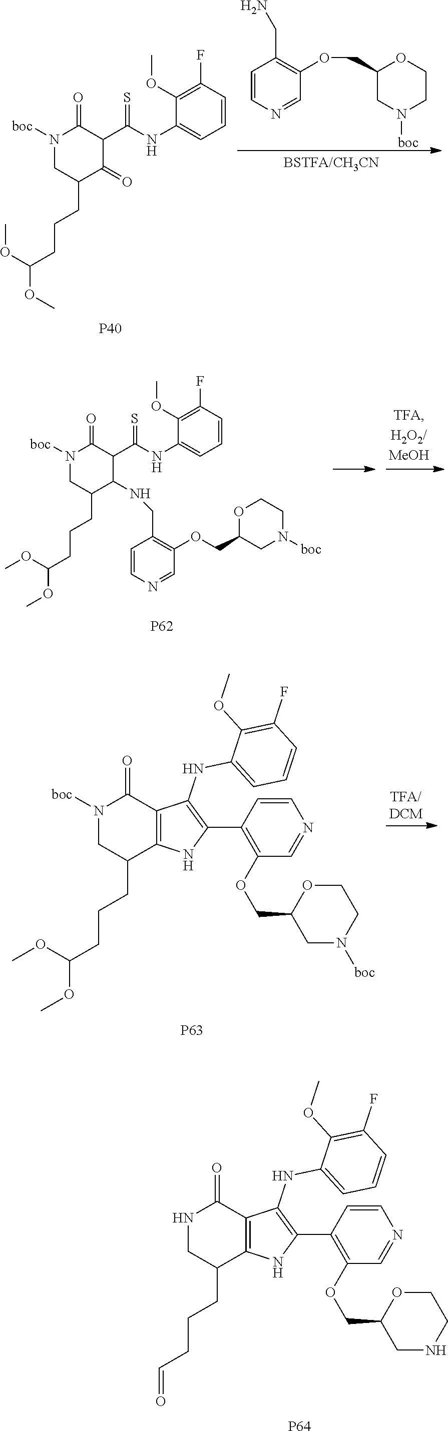

### Preparation 62. tert-Butyl (2S)-2-{[(4-{[(1-(tert-butoxycarbonyl)-5-(4,4-dimethoxybutyl)-3-{[(3-fluoro-2-methoxyphenyl)amino]carbonothioyl}-2-oxopiperidin-4-yl)amino]methyl}pyridin-3-yl)oxy]methyl}morpholine-4-carboxylate (P62)

To a solution of P40 (500 mg, 0.97 mmol, 1.0 eq), P42e (470 mg, 1.5 mmol, 1.5 eq) in dry acetonitrile (10 mL) N,O-bis(trimethylsilyl)trifluoroacetamide (BSTFA, 0.8 mL, 3.0 mmol, 3.0 eq) was added dropwise at 0° C., and then the reaction mixture was stirred at 80° C. for 16 h. The solution was concentrated, the residue was purified by silica gel column chromatography (DCM:MeOH 0-2%) to give P62 (350 mg, 44%) as a yellow oil. ¹H NMR (400 MHz, DMSO-d6), δ: 13.55 (s, 1H), 13.36 (s, 1H), 8.43 (s, 1H), 8.26 (d, J=4.8 Hz, 1H), 7.55 (s, 1H), 7.39 (d, J=4.8 Hz, 1H), 7.18-7.02 (m, 2H), 4.69 (s, 2H), 4.32-4.17 (m, 3H), 4.05-3.67 (m, 9H), 3.45 (d, J=7.9 Hz, 2H), 3.20 (d, J=2.9 Hz, 6H), 3.05 (s, 1H), 2.89 (s, 2H), 1.47 (s, 9H), 1.40 (d, J=8.7 Hz, 9H). LCMS (ESI⁺) m/z 818 [M+H]⁺.

### Preparation 63. tert-Butyl 2-(3-{[(2S)-4-(tert-butoxycarbonyl)morpholin-2-yl]methoxy}pyridin-4-yl)-7-(4,4-dimethoxybutyl)-3-[(3-fluoro-2-methoxyphenyl)amino]-4-oxo-1,4,6,7-tetrahydro-5H-pyrrolo[3,2-c]pyridine-5-carboxylate (P63)

To a solution of P62 (350 mg, 0.4 mmol, 1.0 eq) in methanol (20 mL) TFA (0.03 mL, 0.4 mmol, 1.0 eq) and 35% w/w hydrogen peroxide (0.08 mL, 0.85 mmol, 2.0 eq) were added dropwise at 0° C. The reaction mixture was stirred at 50° C. for 1 h, concentrated and treated with aq. sat. solution of sodium bicarbonate to adjust pH=7. The mixture was extracted with DCM three times. The combined organic layers were dried over Na₂SO₄, filtered, and concentrated to give P63 (150 mg) as a yellow foam. The compound was used for the next step without additional purification. LCMS (ESI⁺) m/z 784 [M+H]⁺.

### Preparation 64. 4-(3-[(3-Fluoro-2-methoxyphenyl)amino]-2-{3-[(2S)-morpholin-2-ylmethoxy]pyridin-4-yl}-4-oxo-4,5,6,7-tetrahydro-1H-pyrrolo[3,2-c]pyridin-7-yl)butanal (P64)

To a solution of P63 (150 mg, 0.2 mmol, 1.0 eq) in DCM (10.0 mL) TFA (0.7 mL, 10 mmol, 50 eq) was added dropwise. The reaction mixture was stirred at rt for 3 h. The solution was treated with a sat. aq. solution of sodium bicarbonate until pH=7 and extracted with DCM/t-BuOH (10 mL, 1:1) three times. The combined organic layers were dried over Na₂SO₄, filtered and concentrated in vacuo to give P64 (100 mg, crude product). LCMS (ESI⁺) m/z 538 [M+H]⁺.

### Synthesis of 3-(3-[(3-fluoro-2-methoxyphenyl)amino]-2-{3-[(2S)-morpholin-2-ylmethoxy]pyridin-4-yl}-4-oxo-4,5,6,7-tetrahydro-1H-pyrrolo[3,2-c]pyridin-7-yl)propanal (P67)

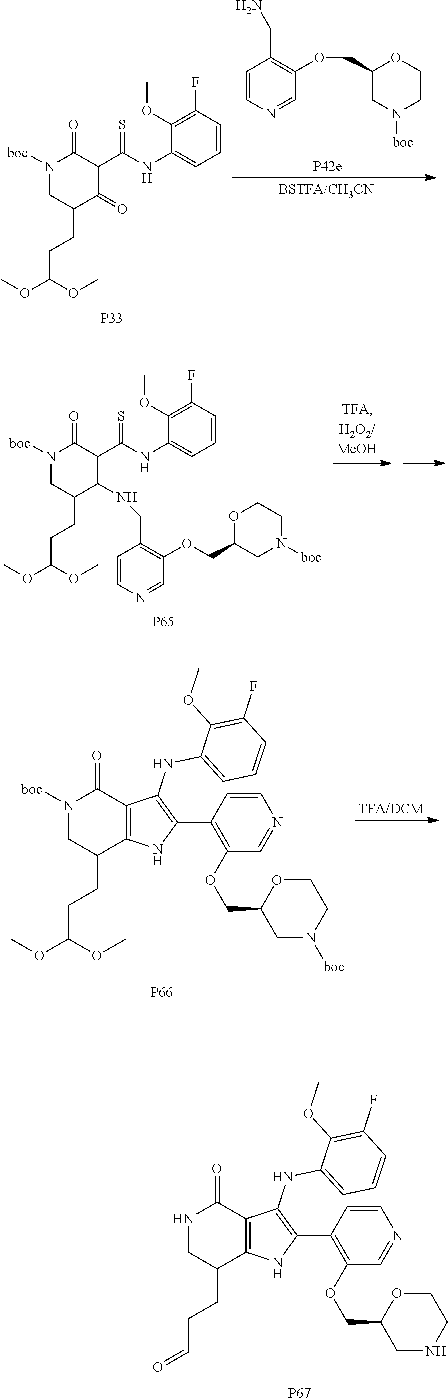

### Preparation 65. tert-Butyl (2S)-2-{[(4-{[(1-(tert-butoxycarbonyl)-5-(3,3-dimethoxypropyl)-3-{[(3-fluoro-2-methoxyphenyl)amino]carbonothioyl}-2-oxopiperidin-4-yl)amino]methyl}pyridin-3-yl)oxy]methyl}morpholine-4-carboxylate (P65)

To a solution of P33 (500 mg, 1.0 mmol, 1.0 eq), P42e (486 mg, 1.5 mmol, 1.5 eq) in dry acetonitrile (10 mL) BSTFA (0.8 mL, 3.0 mmol, 3.0 eq) was added dropwise at 0° C., and then the reaction mixture was stirred at 80° C. for 16 h. The solution was concentrated, the residue was purified by silica gel column chromatography (DCM:MeOH 0-2%) to give P65 (500 mg, 63%) as a yellow oil. ¹H NMR (400 MHz, DMSO-d₆), δ: 13.53 (s, 1H), 13.27 (s, 1H), 8.43 (s, 1H), 8.26 (d, J=4.2 Hz, 1H), 7.57 (s, 1H), 7.38 (d, J=4.0 Hz, 1H), 7.20-6.99 (m, 2H), 4.69 (s, 2H), 4.28 (d, J=39.3 Hz, 3H), 3.96-3.67 (m, 2H), 3.53-3.36 (m, 2H), 3.24-3.14 (m, 6H), 3.07 (s, 1H), 2.89 (s, 1H), 1.65 (s, 2H), 1.47 (s, 9H), 1.39 (s, 9H). LCMS (ESI⁺) m/z 804 [M+H]⁺.

### Preparation 66. tert-Butyl 2-(3-{[(2S)-4-(tert-butoxycarbonyl)morpholin-2-yl]methoxy}pyridin-4-yl)-7-(3,3-dimethoxypropyl)-3-[(3-fluoro-2-methoxyphenyl)amino]-4-oxo-1,4,6,7-tetrahydro-5H-pyrrolo[3,2-c]pyridine-5-carboxylate (P66)

To a solution of P65 (500 mg, 0.6 mmol, 1.0 eq) in methanol (20 mL) TFA (0.05 mL, 0.6 mmol, 1.0 eq) and 35% w/w hydrogen peroxide (0.12 mL, 1.2 mmol, 1.0 eq) were added dropwise at 0° C.

The reaction mixture was stirred at 50° C. for 1 h, concentrated and treated with aq. sat. solution of sodium bicarbonate to adjust pH=7. The mixture was extracted with DCM three times. The combined organic layers were dried over Na₂SO₄, filtered, and concentrated to give P66 (400 mg) as a yellow foam. The compound was used for the next step without additional purification. LCMS (ESI⁺) m/z 770 [M+H].

### Preparation 67. 3-(3-[(3-Fluoro-2-methoxyphenyl)amino]-2-{3-[(2S)-morpholin-2-ylmethoxy]pyridin-4-yl}-4-oxo-4,5,6,7-tetrahydro-1H-pyrrolo[3,2-c]pyridin-7-yl)propanal (P67)

To a solution of P66 (400 mg, 0.5 mmol, 1.0 eq) in DCM (10.0 mL) TFA (2.0 mL, 26 mmol, 50 eq) was added dropwise. The reaction mixture was stirred at rt for 3 h. The solution was treated with a sat. aq. solution of sodium bicarbonate until pH=7 and extracted with DCM/t-BuOH (10 mL, 1:1) three times. The combined organic layers were dried over Na₂SO₄, filtered and concentrated in vacuo to give P67 (300 mg, crude product). LCMS (ESI⁺) m/z 524 [M+H]⁺.

### Synthesis of 3-{2-[3-(azetidin-3-ylmethoxy)pyridin-4-yl]-3-[(3-fluoro-2-methoxyphenyl)amino]-4-oxo-4,5,6,7-tetrahydro-1H-pyrrolo[3,2-c]pyridin-7-yl}propanal (P70)

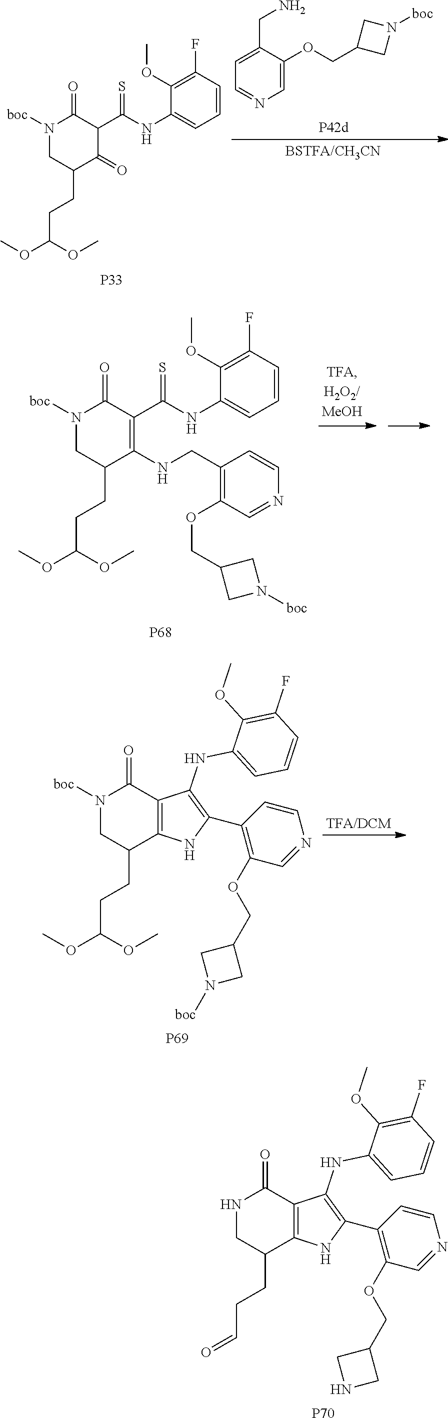

### Preparation 68. tert-Butyl 4-{[(3-{[1-(tert-butoxycarbonyl)azetidin-3-yl]methoxy}pyridin-4-yl)methyl]amino}-3-(3,3-dimethoxypropyl)-5-{[(3-fluoro-2-methoxyphenyl)amino]carbonothioyl}-6-oxo-3,6-dihydropyridine-1(2H)-carboxylate (P68)

To a solution of P33 (700 mg, 1.4 mmol, 1.0 eq), P42d (620 mg, 2.1 mmol, 1.2 eq) in dry acetonitrile (10 mL) BSTFA (1.1 mL, 4.2 mmol, 3.0 eq) was added dropwise at 0° C., and then the reaction mixture was stirred at 80° C. for 16 h. The solution was concentrated, the residue was puriied by silica gel column chromatography (DCM:MeOH 0-2%) to give P68 (700 mg, 65%) as a yellow oil. ¹H NMR (400 MHz, DMSO-d₆), δ: 13.56 (s, 1H), 13.29 (s, 1H), 8.42 (s, 1H), 8.25 (d, J=4.7 Hz, 1H), 7.55 (s, 1H), 7.38 (d, J=4.7 Hz, 1H), 7.19-7.01 (m, 2H), 4.65 (s, 2H), 4.35-4.20 (m, 3H), 4.12-3.89 (m, 3H), 3.81 (s, 3H), 3.73 (s, 2H), 3.21 (s, 3H), 3.18 (s, 3H), 3.09-2.92 (m, 2H), 1.74-1.53 (m, 2H), 1.47 (s, 9H), 1.37 (s, 9H). LCMS (ESI⁺) m/z 774 [M+H]⁺.

### Preparation 69. tert-Butyl 2-(3-{[1-(tert-butoxycarbonyl)azetidin-3-yl]methoxy}pyridin-4-yl)-7-(3,3-dimethoxypropyl)-3-[(3-fluoro-2-methoxyphenyl)amino]-4-oxo-1,4,6,7-tetrahydro-5H-pyrrolo[3,2-c]pyridine-5-carboxylate (P69)

To a solution of P68 (700 mg, 0.9 mmol, 1.0 eq) in methanol (20 mL) TFA (0.07 mL, 0.9 mmol, 1.0 eq) and 35% w/w hydrogen peroxide (0.17 mL, 1.8 mmol, 2.0 eq) were added dropwise at 0° C.

The reaction mixture was stirred at 50° C. for 1 h, concentrated and treated with aq. sat. solution of sodium bicarbonate to adjust pH=7. The mixture was extracted with DCM three times. The combined organic layers were dried over Na₂SO₄, filtered, and concentrated, the residue was purifed by silica gel column chromatography (DCM:MeOH 0-2%) to give P69 (280 mg, 42%) as a yellow foam. LCMS (ESI⁺) m/z 740 [M+H]⁺.

### Preparation 70. 3-{2-[3-(Azetidin-3-ylmethoxy)pyridin-4-yl]-3-[(3-fluoro-2-methoxyphenyl)amino]-4-oxo-4,5,6,7-tetrahydro-1H-pyrrolo[3,2-c]pyridin-7-yl}propanal (P70)

### To a solution of P69 (280 mg, 0.38 mmol, 1.0 eq) in DCM (10.0 mL) TFA (1.5 mL, 20 mmol, 50 eq) was added dropwise. The reaction mixture was stirred at rt for 3 h. The solution was concentrated in vacuo to give P70 (230 mg). LCMS (ESI⁺) m/z 494 [M+H]⁺

### Synthesis of 3-{3-[(3-Fluoro-2-methoxyphenyl)amino]-4-oxo-2-[3-(pyrrolidin-3-ylmethoxy)pyridin-4-yl]-4,5,6,7-tetrahydro-1H-pyrrolo[3,2-c]pyridin-7-yl}propanal (P73)

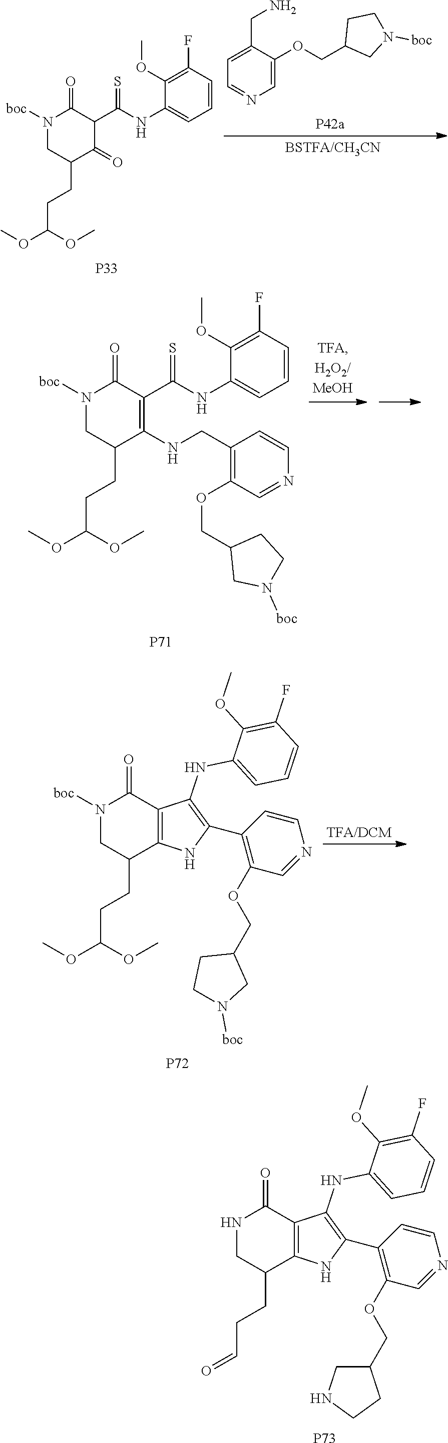

### Preparation 71. tert-Butyl 4-{[(3-{[1-(tert-butoxycarbonyl)pyrrolidin-3-yl]methoxy}pyridin-4-yl)methyl]amino}-3-(3,3-dimethoxypropyl)-5-{[(3-fluoro-2-methoxyphenyl)amino]carbonothioyl}-6-oxo-3,6-dihydropyridine-1(2H)-carboxylate (P71)

To a solution of P33 (370 mg, 1.0 mmol, 1.0 eq), P42a (340 mg, 1.1 mmol, 1.5 eq) in dry acetonitrile (10 mL) BSTFA (0.6 mL, 2.2 mmol, 3.0 eq) was added dropwise at 0° C., and then the reaction mixture was stirred at 80° C. for 16 h. The solution was concentrated, the residue was purified by silica gel column chromatography (DCM:MeOH 0-2%) to give P71 (250 mg, 43%) as a yellow oil. ¹H NMR (400 MHz, DMSO-d₆), δ: 13.53 (s, 1H), 13.27 (s, 1H), 8.44 (s, 1H), 8.25 (d, J=4.6 Hz, 1H), 7.56 (s, 1H), 7.37 (s, 1H), 7.20-7.02 (m, 2H), 4.68 (s, 2H), 4.38-4.17 (m, 2H), 4.15-3.99 (m, 3H), 3.83 (s, 3H), 3.48 (d, J=12.7 Hz, 1H), 3.20 (s, 3H), 3.17 (s, 3H), 3.06 (s, 1H), 1.92 (d, J=14.2 Hz, 3H), 1.80 (s, 1H), 1.63 (s, 2H), 1.47 (s, 9H), 1.38 (s, 9H). LCMS (ESI⁺) m/z 788 [M+H]⁺.

### Preparation 72. tert-Butyl 2-(3-{[1-(tert-butoxycarbonyl)pyrrolidin-3-yl]methoxy}pyridin-4-yl)-7-(3,3-dimethoxypropyl)-3-[(3-fluoro-2-methoxyphenyl)amino]-4-oxo-1,4,6,7-tetrahydro-5H-pyrrolo[3,2-c]pyridine-5-carboxylate (P72)

To a solution of P71 (250 mg, 0.3 mmol, 1.0 eq) in methanol (20 mL) TFA (0.02 mL, 0.3 mmol, 1.0 eq) and 35% w/w hydrogen peroxide (0.06 mL, 0.6 mmol, 2.0 eq) were added dropwise at 0° C. The reaction mixture was stirred at 50° C. for 1 h, concentrated and treated with aq. sat. solution of sodium bicarbonate to adjust pH=7. The mixture was extracted with DCM three times. The combined organic layers were dried over Na₂SO₄, filtered, and concentrated to give P72 (190 mg) as a yellow foam. The compound was used for the next step without additional purification. LCMS (ESI⁺) m/z 755 [M+H].

### Preparation 73. 3-{3-[(3-Fluoro-2-methoxyphenyl)amino]-4-oxo-2-[3-(pyrrolidin-3-ylmethoxy)pyridin-4-yl]-4,5,6,7-tetrahydro-1H-pyrrolo[3,2-c]pyridin-7-yl}propanal (P73)

To a solution of P72 (190 mg, 0.25 mmol, 1.0 eq) in DCM (10.0 mL) TFA (1.0 mL, 12 mmol, 50 eq) was added dropwise. The reaction mixture was stirred at rt for 3 h. The solution was treated with a sat. aq. solution of sodium bicarbonate until pH=7 and extracted with DCM/t-BuOH (10 mL, 1:1) three times. The combined organic layers were dried over Na₂SO₄, filtered and concentrated in vacuo to give P73. LCMS (ESI⁺) m/z 508 [M+H]⁺.

### Synthesis of 3-(3-[(3-fluoro-2-methoxyphenyl)amino]-4-oxo-2-{3-[(2R)-pyrrolidin-2-ylmethoxy]pyridin-4-yl}-4,5,6,7-tetrahydro-1H-pyrrolo[3,2-c]pyridin-7-yl)propanal (P76)

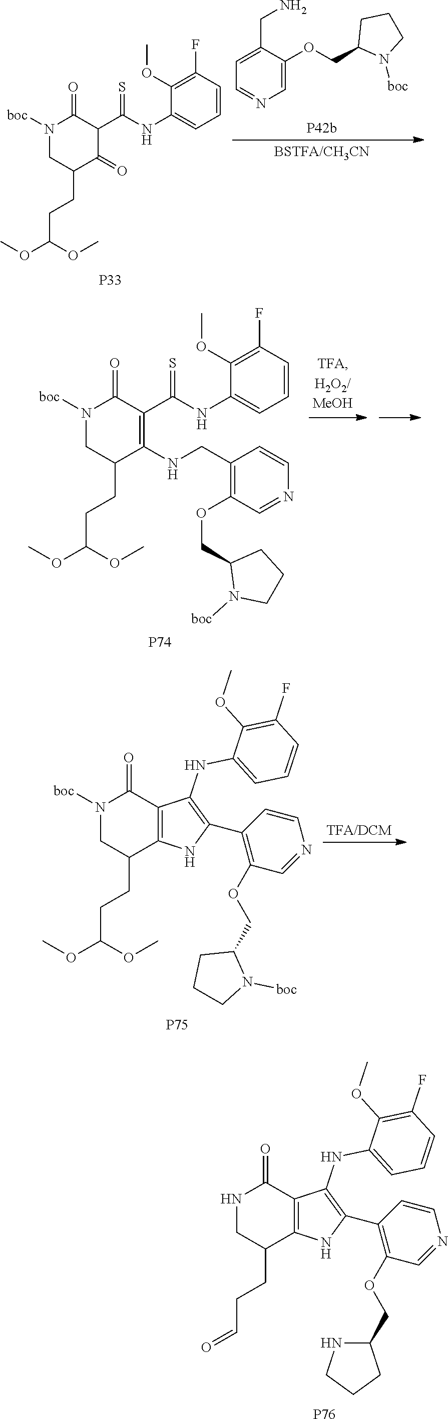

### Preparation 74. tert-Butyl 4-{[(3-{[(2R)-1-(tert-butoxycarbonyl)pyrrolidin-2-yl]methoxy}pyridin-4-yl)methyl]amino}-3-(3,3-dimethoxypropyl)-5-{[(3-fluoro-2-methoxyphenyl)amino]carbonothioyl}-6-oxo-3,6-dihydropyridine-1(2H)-carboxylate (P74)

To a solution of P33 (500 mg, 1.0 mmol, 1.0 eq), P42b (460 mg, 2.1 mmol, 1.5 eq) in dry acetonitrile (10 mL) BSTFA (0.8 mL, 3.0 mmol, 3.0 eq) was added dropwise at 0° C., and then the reaction mixture was stirred at 80° C. for 16 h. The solution was concentrated, the residue was puriied by silica gel column chromatography (DCM:MeOH 0-2%) to give P74 (467 mg, 59%) as a yellow oil. ¹H NMR (400 MHz, DMSO-d₆), δ: 13.53 (s, 1H), 13.27 (s, 1H), 8.44 (s, 1H), 8.25 (d, J=4.6 Hz, 1H), 7.56 (s, 1H), 7.37 (s, 1H), 7.20-7.02 (m, 2H), 4.68 (s, 2H), 4.38-4.17 (m, 2H), 4.15-3.99 (m, 3H), 3.83 (s, 3H), 3.48 (d, J=12.7 Hz, 1H), 3.20 (s, 3H), 3.17 (s, 3H), 3.06 (s, 1H), 1.92 (d, J=14.2 Hz, 3H), 1.80 (s, 1H), 1.63 (s, 2H), 1.47 (s, 9H), 1.38 (s, 9H). LCMS (ESI⁺) m/z 788 [M+H]⁺.

### Preparation 75. tert-Butyl 2-(3-{[(2R)-1-(tert-butoxycarbonyl)pyrrolidin-2-yl]methoxy}pyridin-4-yl)-7-(3,3-dimethoxypropyl)-3-[(3-fluoro-2-methoxyphenyl)amino]-4-oxo-1,4,6,7-tetrahydro-5H-pyrrolo[3,2-c]pyridine-5-carboxylate (P75)

To a solution of P74 (467 mg, 0.6 mmol, 1.0 eq) in methanol (20 mL) TFA (0.05 mL, 0.6 mmol, 1.0 eq) and 35% w/w hydrogen peroxide (0.12 mL, 1.2 mmol, 2.0 eq) were added dropwise at 0° C. The reaction mixture was stirred at 50° C. for 1 h, concentrated and treated with aq. sat. solution of sodium bicarbonate to adjust pH=7. The mixture was extracted with DCM three times. The combined organic layers were dried over Na₂SO₄, filtered, and concentrated to give P75 (450 mg) as a yellow foam. The compound was used for the next step without additional purification. LCMS (ESI⁺) m/z 755 [M+H].

### Preparation 76. 3-(3-[(3-Fluoro-2-methoxyphenyl)amino]-4-oxo-2-{3-[(2R)-pyrrolidin-2-ylmethoxy]pyridin-4-yl}-4,5,6,7-tetrahydro-1H-pyrrolo[3,2-c]pyridin-7-yl)propanal (P76)

To a solution of P75 (450 mg, 0.6 mmol, 1.0 eq) in DCM (10.0 mL) TFA (2.3 mL, 30 mmol, 50 eq) was added dropwise. The reaction mixture was stirred at rt for 3 h. The solution was treated with a sat. aq. solution of sodium bicarbonate until pH=7 and extracted with DCM/t-BuOH (10 mL, 1:1) three times. The combined organic layers were dried over Na₂SO₄, filtered and concentrated in vacuo to give P76 (350 mg, crude product). LCMS (ESI⁺) m/z 508 [M+H]⁺.

### Synthesis of 3-(3-[(3-fluoro-2-methoxyphenyl)amino]-4-oxo-2-{3-[(2S)-pyrrolidin-2-ylmethoxy]pyridin-4-yl}-4,5,6,7-tetrahydro-1H-pyrrolo[3,2-c]pyridin-7-yl)propanal (P79)

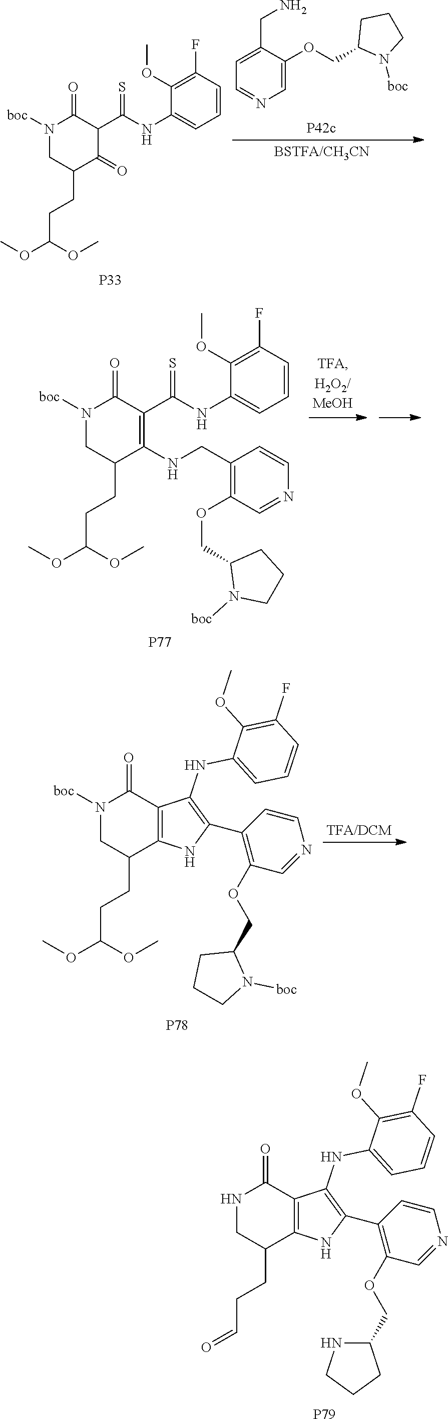

### Preparation 77. tert-Butyl 4-{[(3-{[(2S)-1-(tert-butoxycarbonyl)pyrrolidin-2-yl]methoxy}pyridin-4-yl)methyl]amino}-3-(3,3-dimethoxypropyl)-5-{[(3-fluoro-2-methoxyphenyl)amino]carbonothioyl}-6-oxo-3,6-dihydropyridine-1(2H)-carboxylate (P77)

To a solution of P33 (500 mg, 1.0 mmol, 1.0 eq), P42c (460 mg, 2.1 mmol, 1.5 eq) in dry acetonitrile (10 mL) BSTFA (0.8 mL, 3.0 mmol, 3.0 eq) was added dropwise at 0° C., and then the reaction mixture was stirred at 80° C. for 16 h. The solution was concentrated, the residue was purified by silica gel column chromatography (DCM:MeOH 0-2%) to give P77 (465 mg, 59%) as a yellow oil. ¹H NMR (400 MHz, DMSO-d₆), δ: 13.53 (s, 1H), 13.27 (s, 1H), 8.44 (s, 1H), 8.25 (d, J=4.6 Hz, 1H), 7.56 (s, 1H), 7.37 (s, 1H), 7.20-7.02 (m, 2H), 4.68 (s, 2H), 4.38-4.17 (m, 2H), 4.15-3.99 (m, 3H), 3.83 (s, 3H), 3.48 (d, J=12.7 Hz, 1H), 3.20 (s, 3H), 3.17 (s, 3H), 3.06 (s, 1H), 1.92 (d, J=14.2 Hz, 3H), 1.80 (s, 1H), 1.63 (s, 2H), 1.47 (s, 9H), 1.38 (s, 9H). LCMS (ESI⁺) m/z 788 [M+H]⁺.

### Preparation 78. tert-Butyl 2-(3-{[(2S)-1-(tert-butoxycarbonyl)pyrrolidin-2-yl]methoxy}pyridin-4-yl)-7-(3,3-dimethoxypropyl)-3-[(3-fluoro-2-methoxyphenyl)amino]-4-oxo-1,4,6,7-tetrahydro-5H-pyrrolo[3,2-c]pyridine-5-carboxylate (P78)

To a solution of P77 (465 mg, 0.6 mmol, 1.0 eq) in methanol (20 mL) TFA (0.05 mL, 0.6 mmol, 1.0 eq) and 35% w/w hydrogen peroxide (0.12 mL, 1.2 mmol, 2.0 eq) were added dropwise at 0° C. The reaction mixture was stirred at 50° C. for 1 h, concentrated and treated with aq. sat. solution of sodium bicarbonate to adjust pH=7. The mixture was extracted with DCM three times. The combined organic layers were dried over Na₂SO₄, filtered, and concentrated to give P78 (470 mg) as a yellow foam. The compound was used for the next step without additional purification. LCMS (ESI⁺) m/z 755 [M+H].

### Preparation 79. 3-(3-[(3-Fluoro-2-methoxyphenyl)amino]-4-oxo-2-{3-[(2S)-pyrrolidin-2-ylmethoxy]pyridin-4-yl}-4,5,6,7-tetrahydro-1H-pyrrolo[3,2-c]pyridin-7-yl)propanal (P79)

To a solution of P78 (470 mg, 0.6 mmol, 1.0 eq) in DCM (10.0 mL) TFA (2.3 mL, 30 mmol, 50 eq) was added dropwise. The reaction mixture was stirred at rt for 3 h. The solution was treated with a sat. aq. solution of sodium bicarbonate until pH=7 and extracted with DCM/t-BuOH (10 mL, 1:1) three times. The combined organic layers were dried over Na₂SO₄, filtered and concentrated in vacuo to give P79 (370 mg, crude product). LCMS (ESI⁺) m/z 508 [M+H]⁺.

### Examples of the Final Compound

In the Table 6 presented certain non-limiting examples of the compound of Formula (A).

Table 6. Selected examples of the compound of Formula (A)

TABLE 6
Selected examples of the compound of Formula (A)

| | | [MH]⁺ | [MH]⁺ |
|---|---|---|---|
| # | Structure | calculated | found |
| 1 | 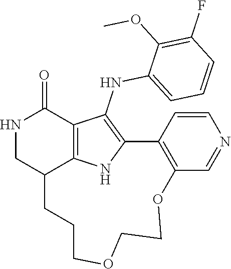 [SMILES: COc1c(F)cccc1Nc1c2[nH]c3c1C(=O)NCC3CCCOCCOc1cnccc1-2] | 453.1938 | 453.3 |
| 2 | 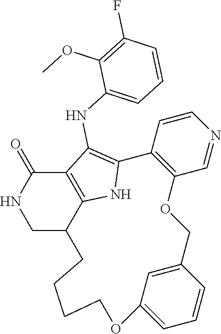 [SMILES: COc1c(F)cccc1Nc1c2[nH]c3c1C(=O)NCC3CCCCOc1cccc(c1)COc1cnccc1-2] | 529.2251 | 529.3 |
| 3 | 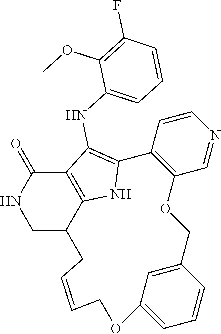 [SMILES: COc1c(F)cccc1Nc1c2[nH]c3c1C(=O)NCC3C/C=C\COc1cccc(c1)COc1cnccc1-2] | 527.2095 | 527.3 |
| 4 | 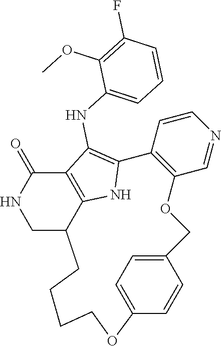 [SMILES: COc1c(F)cccc1Nc1c2[nH]c3c1C(=O)NCC3CCCCOc1ccc(cc1)COc1cnccc1-2] | 529.2251 | 529.3 |
| 5 | 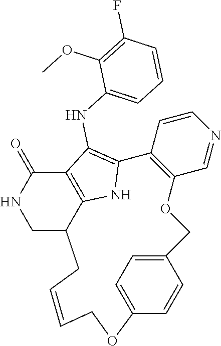 [SMILES: COc1c(F)cccc1Nc1c2[nH]c3c1C(=O)NCC3C/C=C\COc1ccc(cc1)COc1cnccc1-2] | 527.2095 | 527.3 |
| 6 | 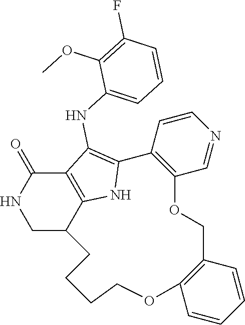 [SMILES: COc1c(F)cccc1Nc1c2[nH]c3c1C(=O)NCC3CCCCOc1ccccc1COc1cnccc1-2] | 529.2251 | 529.3 |
| 7 | 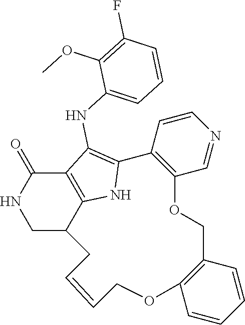 [SMILES: COc1c(F)cccc1Nc1c2[nH]c3c1C(=O)NCC3C/C=C\COc1ccccc1COc1cnccc1-2] | 527.2095 | 527.3 |
| 8 | 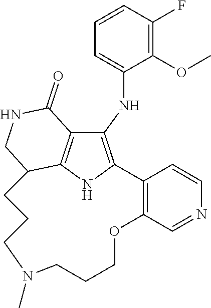 [SMILES: COc1c(F)cccc1Nc1c2[nH]c3c1C(=O)NCC3CCCN(C)CCCOc1cnccc1-2] | 480.2411 | 480.3 |
| 9 | 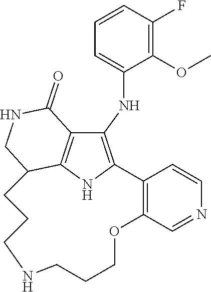 [SMILES: COc1c(F)cccc1Nc1c2[nH]c3c1C(=O)NCC3CCCNCCCOc1cnccc1-2] | 466.2254 | 466.3 |
| 10 | 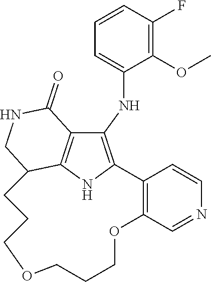 [SMILES: COc1c(F)cccc1Nc1c2[nH]c3c1C(=O)NCC3CCCOCCCOc1cnccc1-2] | 467.2095 | 467.3 |
| 11 | 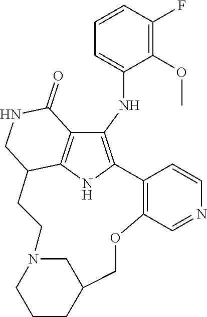 [SMILES: COc1c(F)cccc1Nc1c2[nH]c3c1C(=O)NCC3CCN1CCCC(COc3cnccc3-2)C1] | 492.2411 | 492.3 |
| 12 | 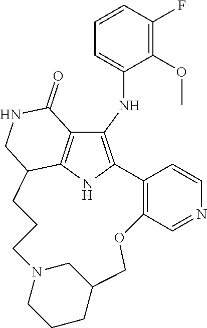 [SMILES: COc1c(F)cccc1Nc1c2[nH]c3c1C(=O)NCC3CCCN1CCCC(COc3cnccc3-2)C1] | 506.2568 | 506 |
| 13 | 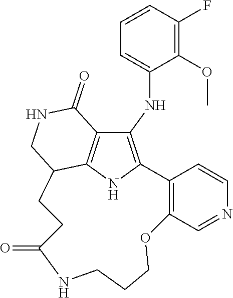 [SMILES: COc1c(F)cccc1Nc1c2[nH]c3c1C(=O)NCC3CCC(=O)NCCCOc1cnccc1-2] | 480.2047 | 480.2 |
| 14 | 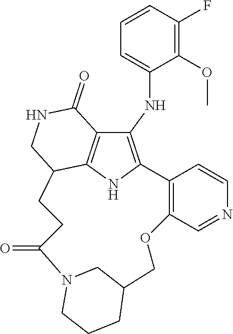 [SMILES: COc1c(F)cccc1Nc1c2[nH]c3c1C(=O)NCC3CCC(=O)N1CCCC(COc3cnccc3-2)C1] | 520.236 | 520.3 |
| 15 | 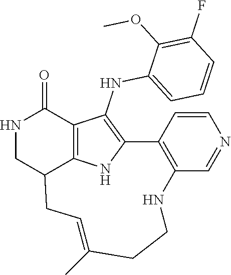 [SMILES: COc1c(F)cccc1Nc1c2[nH]c3c1C(=O)NCC3C/C=C(\C)CCNc1cnccc1-2] | 448.2149 | 448.3 |
| 16 | 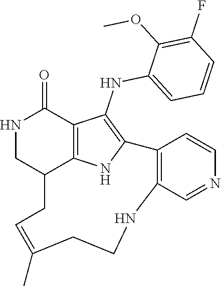 [SMILES: COc1c(F)cccc1Nc1c2[nH]c3c1C(=O)NCC3C/C=C(/C)CCNc1cnccc1-2] | 448.2149 | 448.3 |
| 17 | 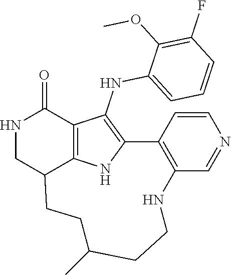 [SMILES: COc1c(F)cccc1Nc1c2[nH]c3c1C(=O)NCC3CCC(C)CCNc1cnccc1-2] | 450.2305 | 450.3 |
| 18 | 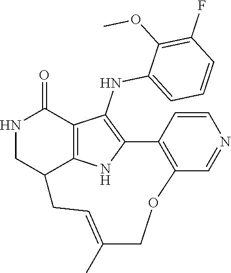 [SMILES: COc1c(F)cccc1Nc1c2[nH]c3c1C(=O)NCC3C/C=C(\C)COc1cnccc1-2] | 435.1832 | 435.2 |
| 19 | 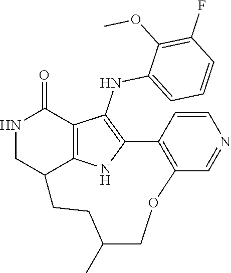 [SMILES: COc1c(F)cccc1Nc1c2[nH]c3c1C(=O)NCC3CCC(C)COc1cnccc1-2] | 437.1989 | 437.2 |
| 20 | 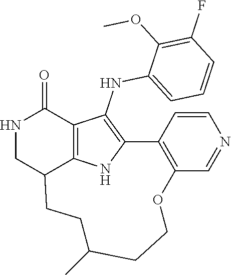 [SMILES: COc1c(F)cccc1Nc1c2[nH]c3c1C(=O)NCC3CCC(C)CCOc1cnccc1-2] | 451.2146 | 451.2 |
| 21 | 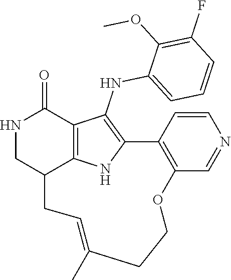 [SMILES: COc1c(F)cccc1Nc1c2[nH]c3c1C(=O)NCC3C/C=C(\C)CCOc1cnccc1-2] | 449.1989 | 449.2 |
| 22 | 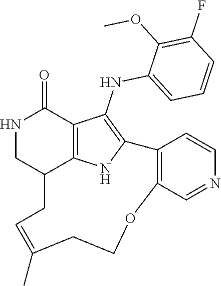 [SMILES: COc1c(F)cccc1Nc1c2[nH]c3c1C(=O)NCC3C/C=C(/C)CCOc1cnccc1-2] | 449.1989 | 449.4 |
| 23 | 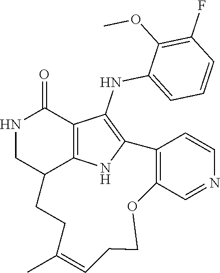 [SMILES: COc1c(F)cccc1Nc1c2[nH]c3c1C(=O)NCC3CC/C(C)=C\CCOc1cnccc1-2] | 463.2146 | 463.3 |
| 24 | 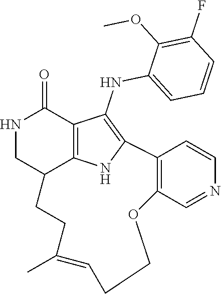 [SMILES: COc1c(F)cccc1Nc1c2[nH]c3c1C(=O)NCC3CC/C(C)=C/CCOc1cnccc1-2] | 463.2146 | 463.3 |
| 25 | 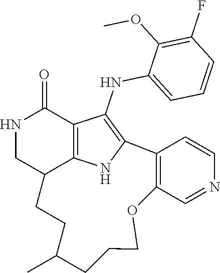 [SMILES: COc1c(F)cccc1Nc1c2[nH]c3c1C(=O)NCC3CCC(C)CCCOc1cnccc1-2] | 465.2302 | 465.3 |
| 26 | 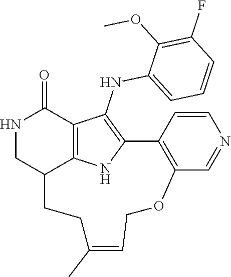 [SMILES: COc1c(F)cccc1Nc1c2[nH]c3c1C(=O)NCC3CC/C(C)=C\COc1cnccc1-2] | 449.1989 | 449.3 |
| 27 | 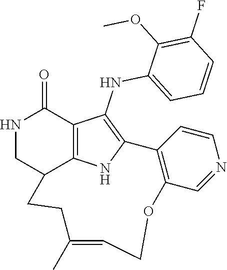 [SMILES: COc1c(F)cccc1Nc1c2[nH]c3c1C(=O)NCC3CC/C(C)=C/COc1cnccc1-2] | 449.1989 | 449.3 |
| 28 | 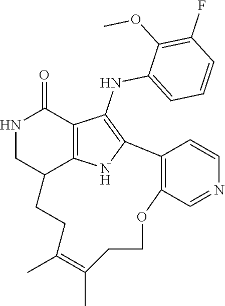 [SMILES: COc1c(F)cccc1Nc1c2[nH]c3c1C(=O)NCC3CC/C(C)=C(/C)CCOc1cnccc1-2] | 477.2302 | 477.5 |
| 29 | 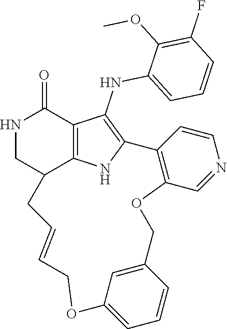 [SMILES: COc1c(F)cccc1Nc1c2[nH]c3c1C(=O)NCC3C/C=C/COc1cccc(c1)COc1cnccc1-2] | 527.2095 | 527.3 |
| 30 | 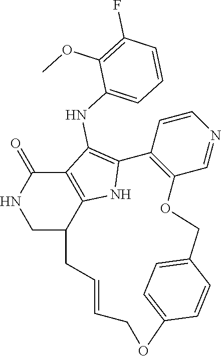 [SMILES: COc1c(F)cccc1Nc1c2[nH]c3c1C(=O)NCC3C/C=C/COc1ccc(cc1)COc1cnccc1-2] | 527.2095 | 527.4 |
| 31 | 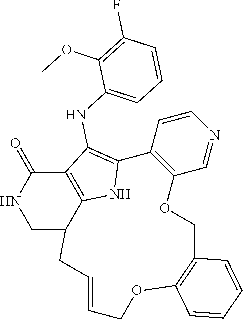 [SMILES: COc1c(F)cccc1Nc1c2[nH]c3c1C(=O)NCC3C/C=C/COc1ccccc1COc1cnccc1-2] | 527.2095 | 527.3 |
| 32 | 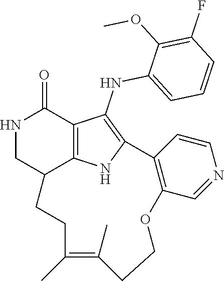 [SMILES: COc1c(F)cccc1Nc1c2[nH]c3c1C(=O)NCC3CC/C(C)=C(\C)CCOc1cnccc1-2] | 477.2302 | 477.5 |
| 33 | 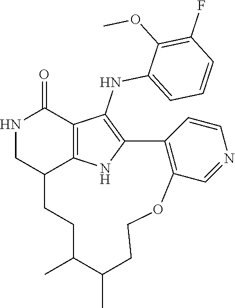 [SMILES: COc1c(F)cccc1Nc1c2[nH]c3c1C(=O)NCC3CCC(C)C(C)CCOc1cnccc1-2] | 479.2458 | 479.3 |
| 34 | 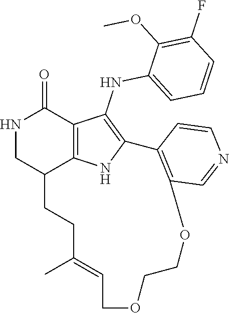 [SMILES: COc1c(F)cccc1Nc1c2[nH]c3c1C(=O)NCC3CC/C(C)=C/COCCOc1cnccc1-2] | 493.2251 | 493.3 |
| 35 | 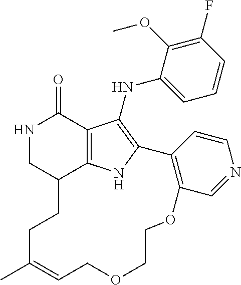 [SMILES: COc1c(F)cccc1Nc1c2[nH]c3c1C(=O)NCC3CC/C(C)=C\COCCOc1cnccc1-2] | 493.2251 | 493.5 |
| 36 | 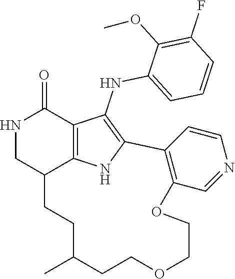 [SMILES: COc1c(F)cccc1Nc1c2[nH]c3c1C(=O)NCC3CCC(C)CCOCCOc1cnccc1-2] | 495.2408 | 495.3 |
| 37 | 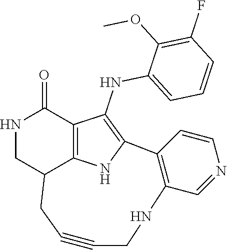 [SMILES: COc1c(F)cccc1Nc1c2[nH]c3c1C(=O)NCC3CC#CCNc1cnccc1-2] | 418.1679 | 418.4 |
| 38 | 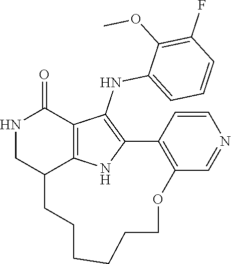 [SMILES: COc1c(F)cccc1Nc1c2[nH]c3c1C(=O)NCC3CCCCCCOc1cnccc1-2] | 451.2146 | 451.3 |
| 39 | 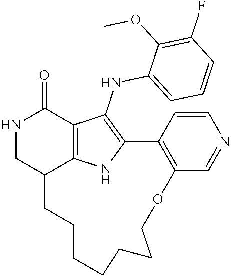 [SMILES: COc1c(F)cccc1Nc1c2[nH]c3c1C(=O)NCC3CCCCCCCOc1cnccc1-2] | 465.2302 | 465.3 |
| 40 | 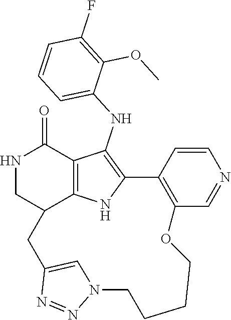 [SMILES: COc1c(F)cccc1Nc1c2[nH]c3c1C(=O)NCC3Cc1cn(nn1)CCCCOc1cnccc1-2] | 504.216 | 504.3 |
| 41 | 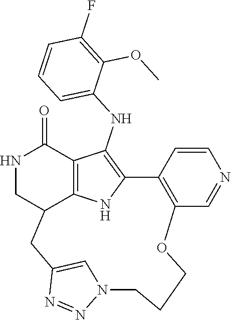 [SMILES: COc1c(F)cccc1Nc1c2[nH]c3c1C(=O)NCC3Cc1cn(nn1)CCCOc1cnccc1-2] | 490.2003 | 490.2 |
| 42 | 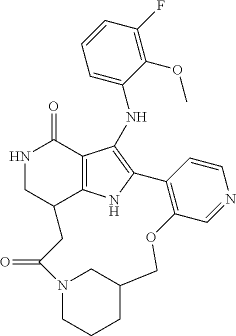 [SMILES: COc1c(F)cccc1Nc1c2[nH]c3c1C(=O)NCC3CC(=O)N1CCCC(COc3cnccc3-2)C1] | 506.2203 | 506.3 |
| 43 | 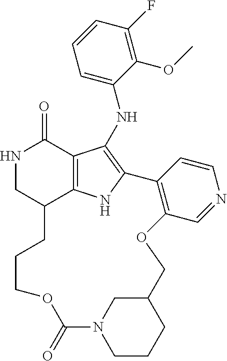 [SMILES: COc1c(F)cccc1Nc1c2[nH]c3c1C(=O)NCC3CCCOC(=O)N1CCCC(COc3cnccc3-2)C1] | 550.2466 | 550.3 |
| 44 | 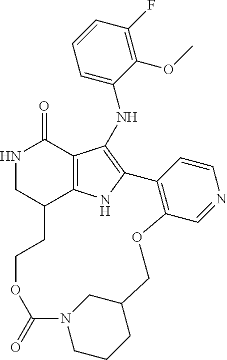 [SMILES: COc1c(F)cccc1Nc1c2[nH]c3c1C(=O)NCC3CCOC(=O)N1CCCC(COc3cnccc3-2)C1] | 536.2309 | 536.3 |
| 45 | 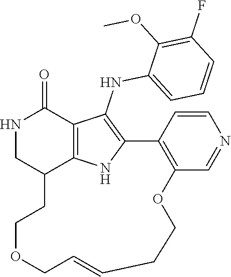 [SMILES: COc1c(F)cccc1Nc1c2[nH]c3c1C(=O)NCC3CCOC/C=C/CCOc1cnccc1-2] | 479.2095 | 479.3 |
| 46 | 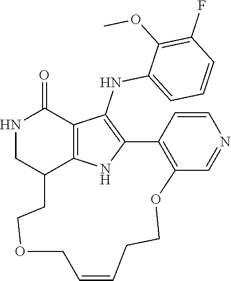 [SMILES: COc1c(F)cccc1Nc1c2[nH]c3c1C(=O)NCC3CCOC/C=C\CCOc1cnccc1-2] | 479.2095 | 479.3 |
| 47 | 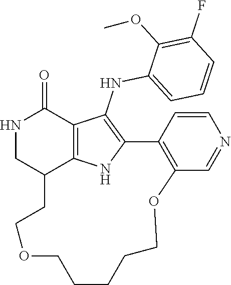 [SMILES: COc1c(F)cccc1Nc1c2[nH]c3c1C(=O)NCC3CCOCCCCCOc1cnccc1-2] | 481.2251 | 481.3 |
| 48 | 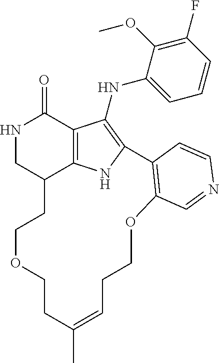 [SMILES: COc1c(F)cccc1Nc1c2[nH]c3c1C(=O)NCC3CCOCC/C(C)=C\CCOc1cnccc1-2] | 507.2408 | 507.3 |
| 49 | 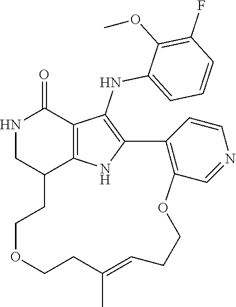 [SMILES: COc1c(F)cccc1Nc1c2[nH]c3c1C(=O)NCC3CCOCC/C(C)=C/CCOc1cnccc1-2] | 507.2408 | 507.3 |
| 50 | 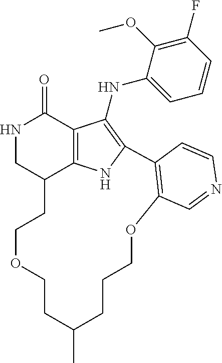 [SMILES: COc1c(F)cccc1Nc1c2[nH]c3c1C(=O)NCC3CCOCCC(C)CCCOc1cnccc1-2] | 509.2564 | 509.3 |
| 51 | 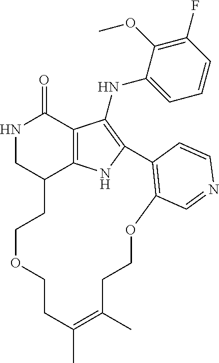 [SMILES: COc1c(F)cccc1Nc1c2[nH]c3c1C(=O)NCC3CCOCC/C(C)=C(/C)CCOc1cnccc1-2] | 521.2564 | 521.3 |
| 52 | 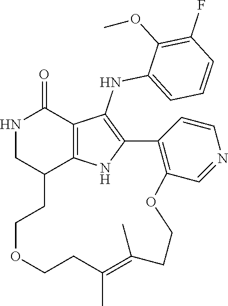 [SMILES: COc1c(F)cccc1Nc1c2[nH]c3c1C(=O)NCC3CCOCC/C(C)=C(\C)CCOc1cnccc1-2] | 521.2564 | 521.3 |
| 53 | 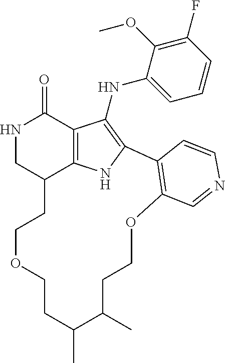 [SMILES: COc1c(F)cccc1Nc1c2[nH]c3c1C(=O)NCC3CCOCCC(C)C(C)CCOc1cnccc1-2] | 523.272 | 523.3 |
| 54 | 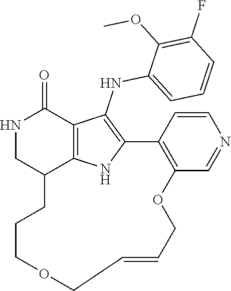 [SMILES: COc1c(F)cccc1Nc1c2[nH]c3c1C(=O)NCC3CCCOC/C=C/COc1cnccc1-2] | 479.2095 | 479.3 |
| 55 | 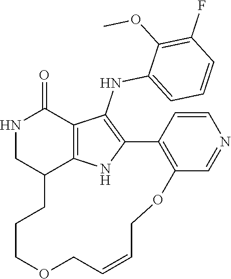 [SMILES: COc1c(F)cccc1Nc1c2[nH]c3c1C(=O)NCC3CCCOC/C=C\COc1cnccc1-2] | 479.2095 | 479.3 |
| 56 | 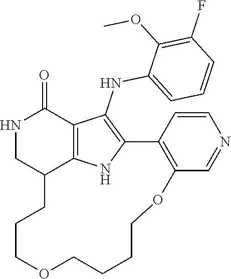 [SMILES: COc1c(F)cccc1Nc1c2[nH]c3c1C(=O)NCC3CCCOCCCCOc1cnccc1-2] | 481.2251 | 481.3 |
| 57 | 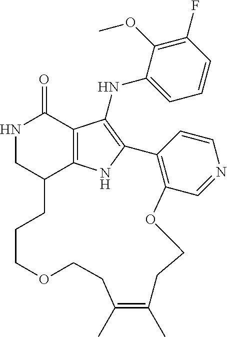 [SMILES: COc1c(F)cccc1Nc1c2[nH]c3c1C(=O)NCC3CCCOCC/C(C)=C(/C)CCOc1cnccc1-2] | 535.272 | 535.4 |
| 58 | 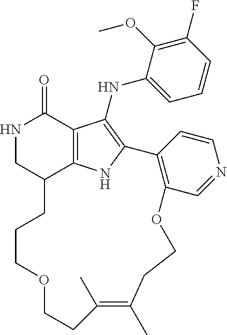 [SMILES: COc1c(F)cccc1Nc1c2[nH]c3c1C(=O)NCC3CCCOCC/C(C)=C(\C)CCOc1cnccc1-2] | 535.272 | 535.3 |
| 59 | 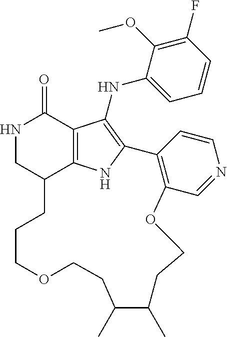 [SMILES: COc1c(F)cccc1Nc1c2[nH]c3c1C(=O)NCC3CCCOCCC(C)C(C)CCOc1cnccc1-2] | 537.2877 | 537.4 |
| 60 | 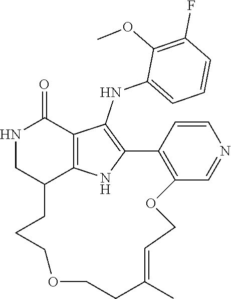 [SMILES: COc1c(F)cccc1Nc1c2[nH]c3c1C(=O)NCC3CCCOCC/C(C)=C/COc1cnccc1-2] | 507.2408 | 507.3 |
| 61 | 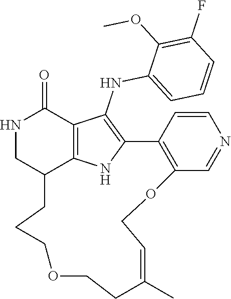 [SMILES: COc1c(F)cccc1Nc1c2[nH]c3c1C(=O)NCC3CCCOCC/C(C)=C\COc1cnccc1-2] | 507.2408 | 507.3 |
| 62 | 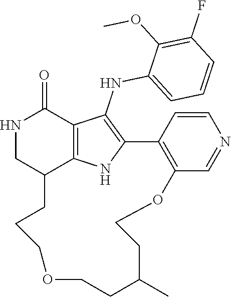 [SMILES: COc1c(F)cccc1Nc1c2[nH]c3c1C(=O)NCC3CCCOCCC(C)CCOc1cnccc1-2] | 509.2564 | 509.5 |
| 63 | 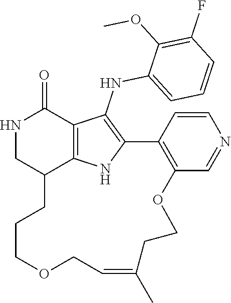 [SMILES: COc1c(F)cccc1Nc1c2[nH]c3c1C(=O)NCC3CCCOC/C=C(/C)CCOc1cnccc1-2] | 507.2408 | 507.3 |
| 64 | 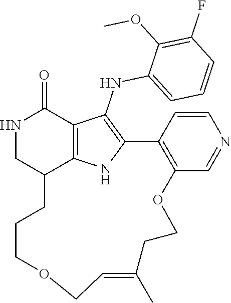 [SMILES: COc1c(F)cccc1Nc1c2[nH]c3c1C(=O)NCC3CCCOC/C=C(\C)CCOc1cnccc1-2] | 507.2408 | 507.3 |
| 65 | 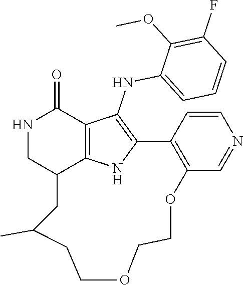 [SMILES: COc1c(F)cccc1Nc1c2[nH]c3c1C(=O)NCC3CC(C)CCOCCOc1cnccc1-2] | 481.2251 | 481.3 |
| 66 | 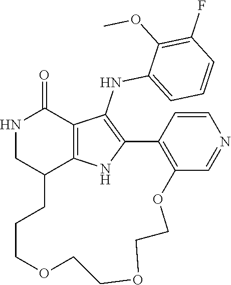 [SMILES: COc1c(F)cccc1Nc1c2[nH]c3c1C(=O)NCC3CCCOCCOCCOc1cnccc1-2] | 497.22 | 497.3 |
| 67 | 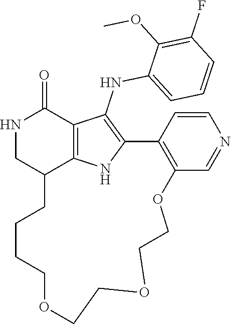 [SMILES: COc1c(F)cccc1Nc1c2[nH]c3c1C(=O)NCC3CCCCOCCOCCOc1cnccc1-2] | 511.2357 | 511.3 |
| 68 | 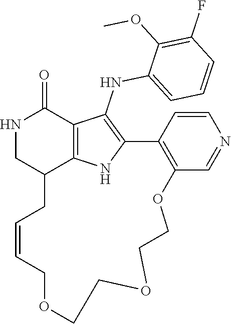 [SMILES: COc1c(F)cccc1Nc1c2[nH]c3c1C(=O)NCC3C/C=C\COCCOCCOc1cnccc1-2] | 509.22 | 509.3 |
| 69 | 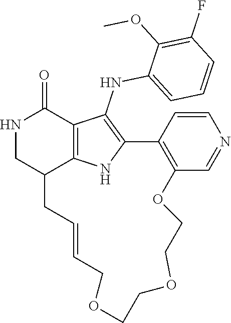 [SMILES: COc1c(F)cccc1Nc1c2[nH]c3c1C(=O)NCC3C/C=C/COCCOCCOc1cnccc1-2] | 509.22 | 509.3 |
| 70 | 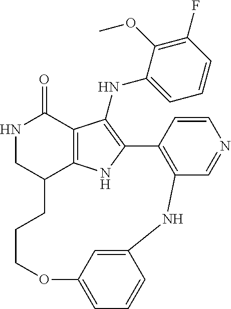 [SMILES: COc1c(F)cccc1Nc1c2[nH]c3c1C(=O)NCC3CCCOc1cccc(c1)Nc1cnccc1-2] | 500.2098 | 500.3 |
| 71 | 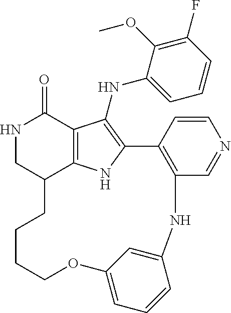 [SMILES: COc1c(F)cccc1Nc1c2[nH]c3c1C(=O)NCC3CCCCOc1cccc(c1)Nc1cnccc1-2] | 514.2254 | 514.3 |
| 72 | 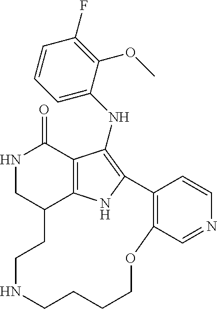 [SMILES: COc1c(F)cccc1Nc1c2[nH]c3c1C(=O)NCC3CCNCCCCOc1cnccc1-2] | 466.2254 | 466.3 |
| 73 | 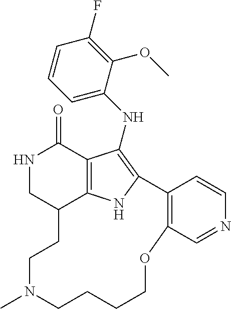 [SMILES: COc1c(F)cccc1Nc1c2[nH]c3c1C(=O)NCC3CCN(C)CCCCOc1cnccc1-2] | 480.2411 | 480.3 |
| 74 | 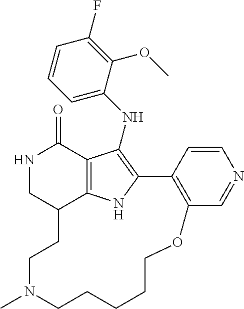 [SMILES: COc1c(F)cccc1Nc1c2[nH]c3c1C(=O)NCC3CCN(C)CCCCCOc1cnccc1-2] | 494.2568 | 494.4 |
| 75 | 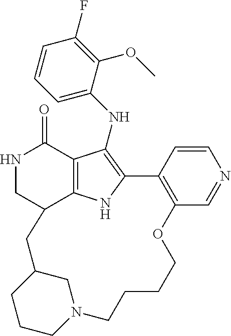 [SMILES: COc1c(F)cccc1Nc1c2[nH]c3c1C(=O)NCC3CC1CCCN(CCCCOc3cnccc3-2)C1] | 520.2724 | 520.5 |
| 76 | 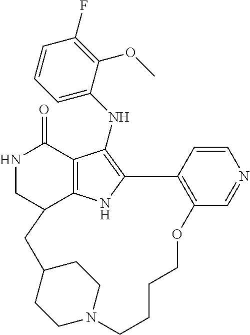 [SMILES: COc1c(F)cccc1Nc1c2[nH]c3c1C(=O)NCC3CC1CCN(CCCCOc3cnccc3-2)CC1] | 520.2724 | 520.5 |
| 77 | 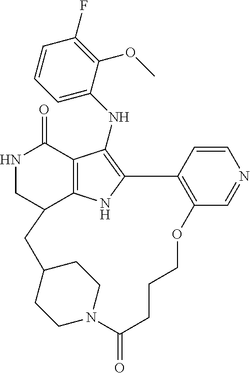 [SMILES: COc1c(F)cccc1Nc1c2[nH]c3c1C(=O)NCC3CC1CCN(CC1)C(=O)CCCOc1cnccc1-2] | 534.2517 | 534.4 |
| 78 | 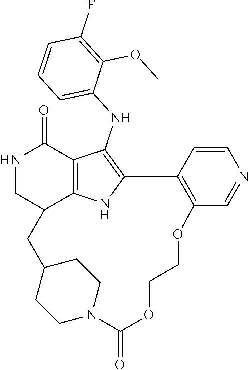 [SMILES: COc1c(F)cccc1Nc1c2[nH]c3c1C(=O)NCC3CC1CCN(CC1)C(=O)OCCOc1cnccc1-2] | 536.2309 | 536.3 |
| 79 | 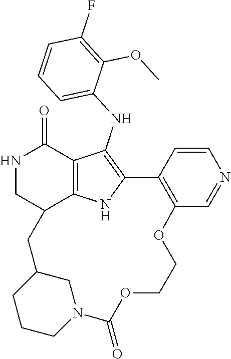 [SMILES: COc1c(F)cccc1Nc1c2[nH]c3c1C(=O)NCC3CC1CCCN(C1)C(=O)OCCOc1cnccc1-2] | 536.2309 | 536.3 |
| 80 | 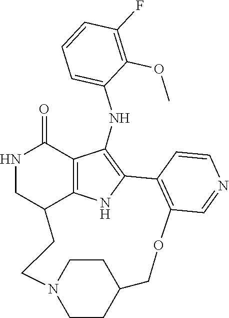 [SMILES: COc1c(F)cccc1Nc1c2[nH]c3c1C(=O)NCC3CCN1CCC(CC1)COc1cnccc1-2] | 492.2411 | 492.3 |
| 81 | 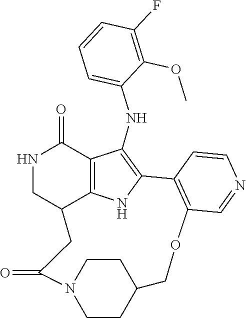 [SMILES: COc1c(F)cccc1Nc1c2[nH]c3c1C(=O)NCC3CC(=O)N1CCC(CC1)COc1cnccc1-2] | 506.2203 | 506.3 |
| 82 | 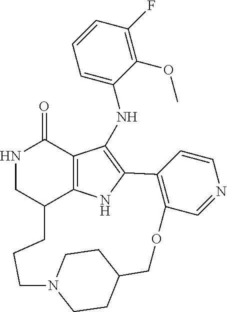 [SMILES: COc1c(F)cccc1Nc1c2[nH]c3c1C(=O)NCC3CCCN1CCC(CC1)COc1cnccc1-2] | 506.2568 | 506.3 |
| 83 | 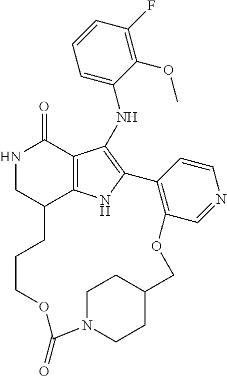 [SMILES: COc1c(F)cccc1Nc1c2[nH]c3c1C(=O)NCC3CCCOC(=O)N1CCC(CC1)COc1cnccc1-2] | 550.2466 | 550.3 |
| 84 | 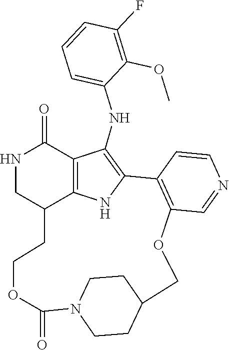 [SMILES: COc1c(F)cccc1Nc1c2[nH]c3c1C(=O)NCC3CCOC(=O)N1CCC(CC1)COc1cnccc1-2] | 536.2309 | 536.3 |
| 85 | 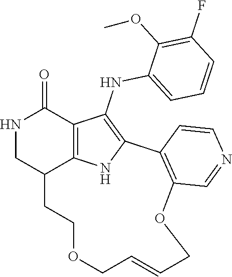 [SMILES: COc1c(F)cccc1Nc1c2[nH]c3c1C(=O)NCC3CCOC/C=C/COc1cnccc1-2] | 465.1938 | 465.2 |
| 86 | 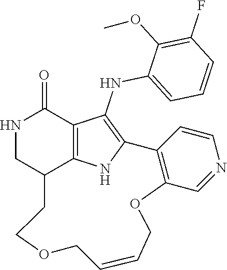 [SMILES: COc1c(F)cccc1Nc1c2[nH]c3c1C(=O)NCC3CCOC/C=C\COc1cnccc1-2] | 465.1938 | 465.3 |
| 87 | 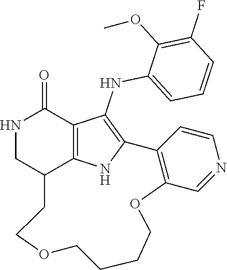 [SMILES: COc1c(F)cccc1Nc1c2[nH]c3c1C(=O)NCC3CCOCCCCOc1cnccc1-2] | 467.2095 | 467.3 |
| 88 | 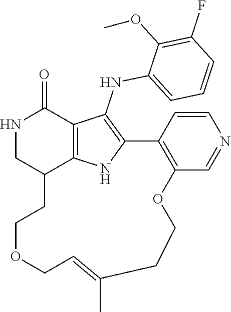 [SMILES: COc1c(F)cccc1Nc1c2[nH]c3c1C(=O)NCC3CCOC/C=C(\C)CCOc1cnccc1-2] | 493.2251 | 493.3 |
| 89 | 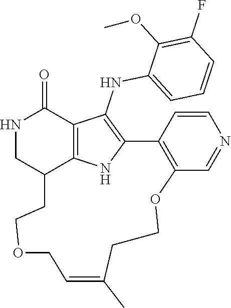 [SMILES: COc1c(F)cccc1Nc1c2[nH]c3c1C(=O)NCC3CCOC/C=C(/C)CCOc1cnccc1-2] | 493.2251 | 493.3 |
| 90 | 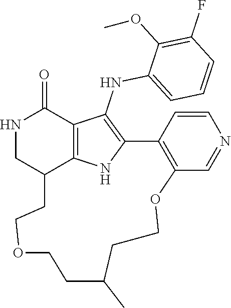 [SMILES: COc1c(F)cccc1Nc1c2[nH]c3c1C(=O)NCC3CCOCCC(C)CCOc1cnccc1-2] | 495.2408 | 495.3 |
| 91 | 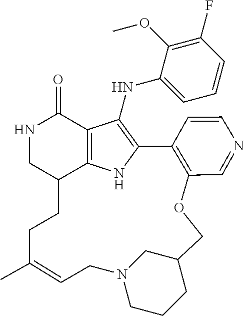 [SMILES: COc1c(F)cccc1Nc1c2[nH]c3c1C(=O)NCC3CC/C(C)=C\CN1CCCC(COc3cnccc3-2)C1] | 546.288 | 546.5 |
| 92 | 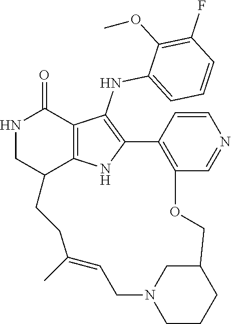 [SMILES: COc1c(F)cccc1Nc1c2[nH]c3c1C(=O)NCC3CC/C(C)=C/CN1CCCC(COc3cnccc3-2)C1] | 546.288 | 546.5 |
| 93 | 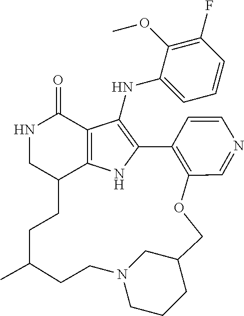 [SMILES: COc1c(F)cccc1Nc1c2[nH]c3c1C(=O)NCC3CCC(C)CCN1CCCC(COc3cnccc3-2)C1] | 548.3037 | 548.3 |
| 94 | 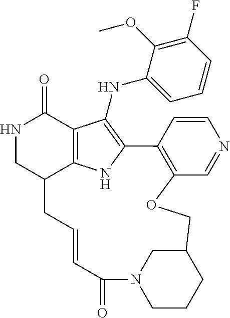 [SMILES: COc1c(F)cccc1Nc1c2[nH]c3c1C(=O)NCC3C/C=C/C(=O)N1CCCC(COc3cnccc3-2)C1] | 532.236 | 532.3 |
| 95 | 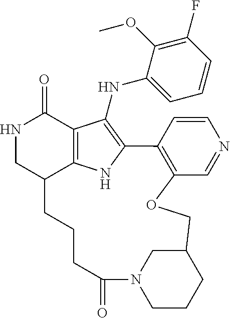 [SMILES: COc1c(F)cccc1Nc1c2[nH]c3c1C(=O)NCC3CCCC(=O)N1CCCC(COc3cnccc3-2)C1] | 534.2517 | 534.3 |
| 96 | 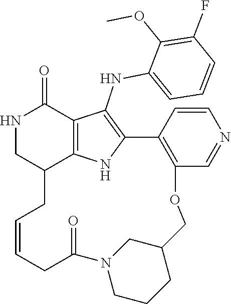 [SMILES: COc1c(F)cccc1Nc1c2[nH]c3c1C(=O)NCC3C/C=C\CC(=O)N1CCCC(COc3cnccc3-2)C1] | 546.2517 | 546.3 |
| 97 | 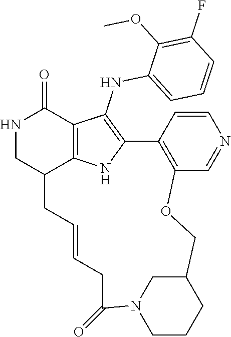 [SMILES: COc1c(F)cccc1Nc1c2[nH]c3c1C(=O)NCC3C/C=C/CC(=O)N1CCCC(COc3cnccc3-2)C1] | 546.2517 | 546.5 |
| 98 | 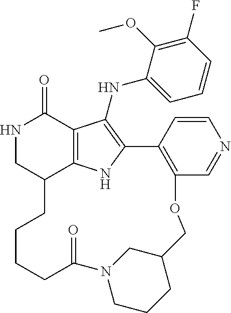 [SMILES: COc1c(F)cccc1Nc1c2[nH]c3c1C(=O)NCC3CCCCC(=O)N1CCCC(COc3cnccc3-2)C1] | 548.2673 | 548.5 |
| 99 | 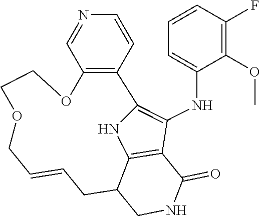 [SMILES: COc1c(F)cccc1Nc1c2[nH]c3c1C(=O)NCC3C/C=C/COCCOc1cnccc1-2] | 465.1938 | 465.4 |
| 100 | 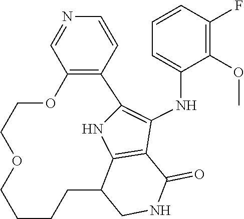 [SMILES: COc1c(F)cccc1Nc1c2[nH]c3c1C(=O)NCC3CCCCOCCOc1cnccc1-2] | 467.2095 | 467.5 |
| 101 | 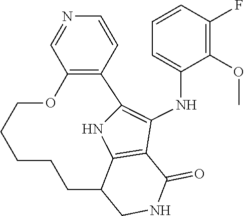 [SMILES: COc1c(F)cccc1Nc1c2[nH]c3c1C(=O)NCC3CCCCCOc1cnccc1-2] | 437.1989 | 437.2 |
| 102 | 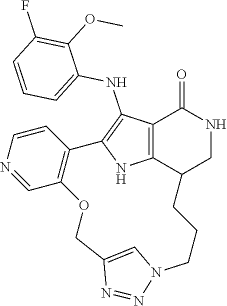 [SMILES: COc1c(F)cccc1Nc1c2[nH]c3c1C(=O)NCC3CCCn1cc(nn1)COc1cnccc1-2] | 490.2003 | 490.2 |
| 103 | 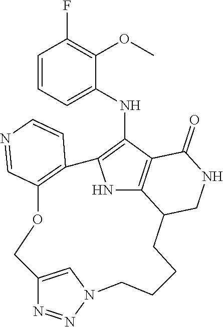 [SMILES: COc1c(F)cccc1Nc1c2[nH]c3c1C(=O)NCC3CCCCn1cc(nn1)COc1cnccc1-2] | 504.216 | 504.3 |
| 104 | 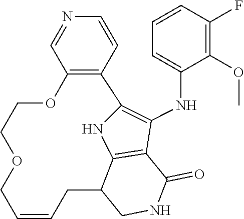 [SMILES: COc1c(F)cccc1Nc1c2[nH]c3c1C(=O)NCC3C/C=C\COCCOc1cnccc1-2] | 465.1938 | 465.3 |
| 105 | 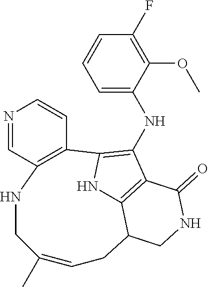 [SMILES: COc1c(F)cccc1Nc1c2[nH]c3c1C(=O)NCC3C/C=C(/C)CNc1cnccc1-2] | 434.1992 | 434.3 |
| 106 | 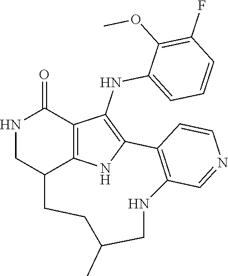 [SMILES: COc1c(F)cccc1Nc1c2[nH]c3c1C(=O)NCC3CCC(C)CNc1cnccc1-2] | 436.2149 | 436.4 |
| 107 | 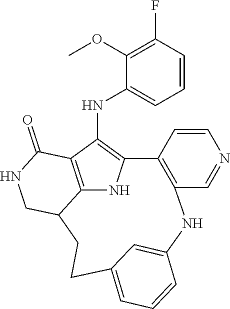 [SMILES: COc1c(F)cccc1Nc1c2[nH]c3c1C(=O)NCC3CCc1cccc(c1)Nc1cnccc1-2] | 470.1992 | 470.3 |
| 108 | 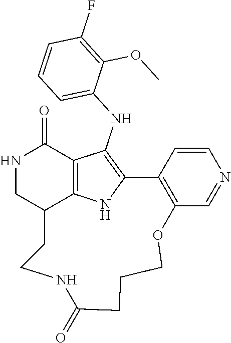 [SMILES: COc1c(F)cccc1Nc1c2[nH]c3c1C(=O)NCC3CCNC(=O)CCCOc1cnccc1-2] | 480.2047 | 480.3 |
| 109 | 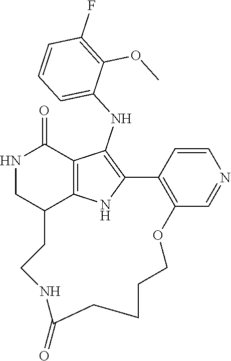 [SMILES: COc1c(F)cccc1Nc1c2[nH]c3c1C(=O)NCC3CCNC(=O)CCCCOc1cnccc1-2] | 494.2203 | 494.3 |
| 110 | 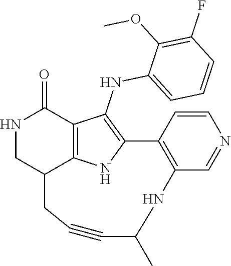 [SMILES: COc1c(F)cccc1Nc1c2[nH]c3c1C(=O)NCC3CC#CC(C)Nc1cnccc1-2] | 432.1836 | 432.3 |
| 111 | 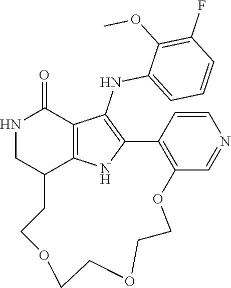 [SMILES: COc1c(F)cccc1Nc1c2[nH]c3c1C(=O)NCC3CCOCCOCCOc1cnccc1-2] | 483.2044 | 483.3 |
| 112 | 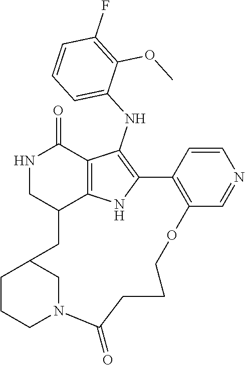 [SMILES: COc1c(F)cccc1Nc1c2[nH]c3c1C(=O)NCC3CC1CCCN(C1)C(=O)CCCOc1cnccc1-2] | 534.2517 | 534.3 |
| 113 | 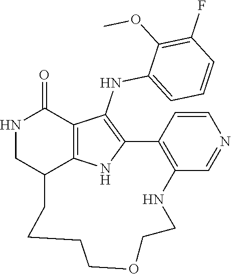 [SMILES: COc1c(F)cccc1Nc1c2[nH]c3c1C(=O)NCC3CCCCOCCNc1cnccc1-2] | 466.2254 | 466.3 |
| 114 | 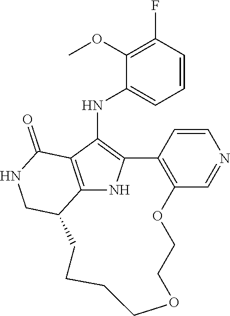 [SMILES: COc1c(F)cccc1Nc1c2[nH]c3c1C(=O)NC[C@H]3CCCCOCCOc1cnccc1-2] | 467.2095 | 467.3 |
| 115 | 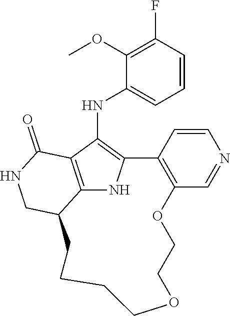 [SMILES: COc1c(F)cccc1Nc1c2[nH]c3c1C(=O)NC[C@@H]3CCCCOCCOc1cnccc1-2] | 467.2095 | 467.3 |
| 116 | 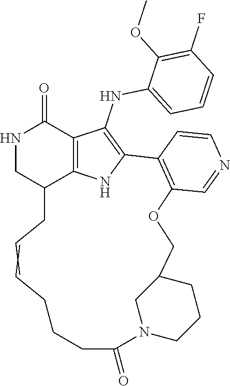 [SMILES: COc1c(F)cccc1Nc1c2[nH]c3c1C(=O)NCC3CC=CCCCC(=O)N1CCCC(COc3cnccc3-2)C1] | 574.283 | 574.4 |
| 117 | 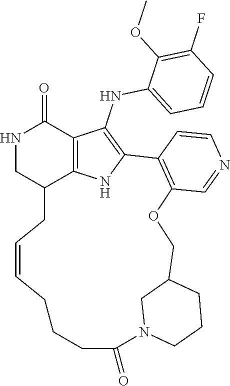 [SMILES: COc1c(F)cccc1Nc1c2[nH]c3c1C(=O)NCC3C/C=C\CCCC(=O)N1CCCC(COc3cnccc3-2)C1] | 574.283 | 574.5 |
| 118 | 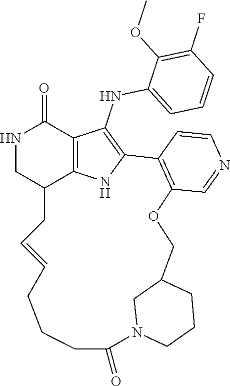 [SMILES: COc1c(F)cccc1Nc1c2[nH]c3c1C(=O)NCC3C/C=C/CCCC(=O)N1CCCC(COc3cnccc3-2)C1] | 574.283 | 574.4 |
| 119 | 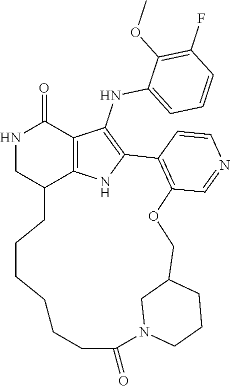 [SMILES: COc1c(F)cccc1Nc1c2[nH]c3c1C(=O)NCC3CCCCCCC(=O)N1CCCC(COc3cnccc3-2)C1] | 576.299 | 576 |
| 120 | 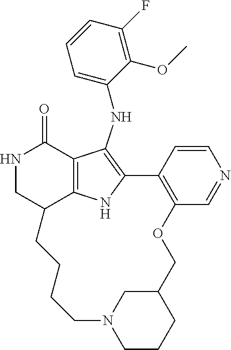 [SMILES: COc1c(F)cccc1Nc1c2[nH]c3c1C(=O)NCC3CCCCN1CCCC(COc3cnccc3-2)C1] | 520.272 | 520.5 |
| 121 | 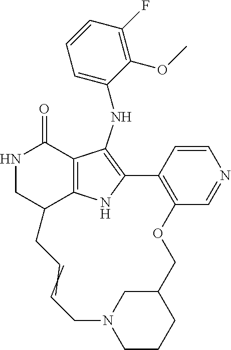 [SMILES: COc1c(F)cccc1Nc1c2[nH]c3c1C(=O)NCC3CC=CCN1CCCC(COc3cnccc3-2)C1] | 518.257 | 518.3 |
| 122 | 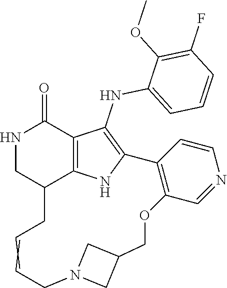 [SMILES: COc1c(F)cccc1Nc1c2[nH]c3c1C(=O)NCC3CC=CCN1CC(COc3cnccc3-2)C1] | 490.225 | 490.3 |
| 123 | 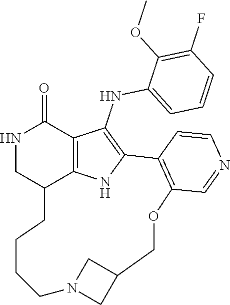 [SMILES: COc1c(F)cccc1Nc1c2[nH]c3c1C(=O)NCC3CCCCN1CC(COc3cnccc3-2)C1] | 492.241 | 492.5 |
| 124 | 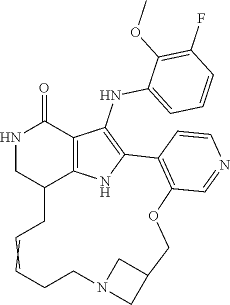 [SMILES: COc1c(F)cccc1Nc1c2[nH]c3c1C(=O)NCC3CC=CCCN1CC(COc3cnccc3-2)C1] | 504.241 | 504.3 |
| 125 | 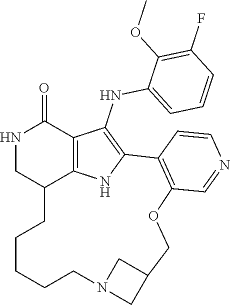 [SMILES: COc1c(F)cccc1Nc1c2[nH]c3c1C(=O)NCC3CCCCCN1CC(COc3cnccc3-2)C1] | 506.257 | 506.5 |
| 126 | 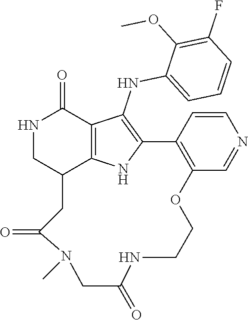 [SMILES: COc1c(F)cccc1Nc1c2[nH]c3c1C(=O)NCC3CC(=O)N(C)CC(=O)NCCOc1cnccc1-2] | 523.211 | 523.3 |
| 127 | 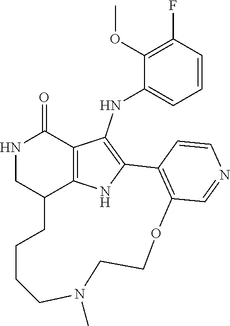 [SMILES: COc1c(F)cccc1Nc1c2[nH]c3c1C(=O)NCC3CCCCN(C)CCOc1cnccc1-2] | 480.241 | 481 |
| 128 | 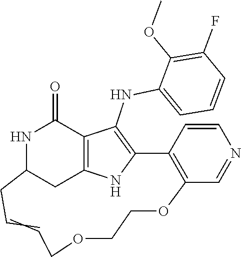 [SMILES: COc1c(F)cccc1Nc1c2[nH]c3c1C(=O)NC(CC=CCOCCOc1cnccc1-2)C3] | 465.194 | 465.3 |
| 129 | 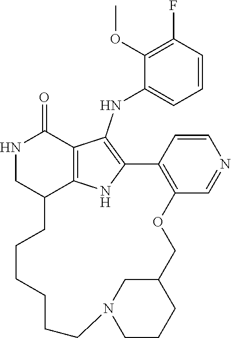 [SMILES: COc1c(F)cccc1Nc1c2[nH]c3c1C(=O)NCC3CCCCCCN1CCCC(COc3cnccc3-2)C1] | 548.304 | 548.5 |
| 130 | 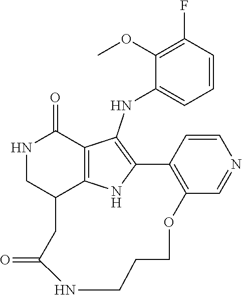 [SMILES: COc1c(F)cccc1Nc1c2[nH]c3c1C(=O)NCC3CC(=O)NCCCOc1cnccc1-2] | 466.189 | 466.3 |
| 131 | 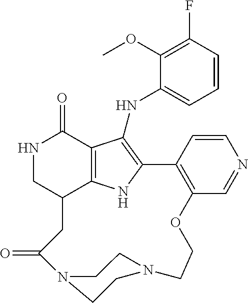 [SMILES: COc1c(F)cccc1Nc1c2[nH]c3c1C(=O)NCC3CC(=O)N1CCN(CCOc3cnccc3-2)CC1] | 521.231 | 521.3 |
| 132 | 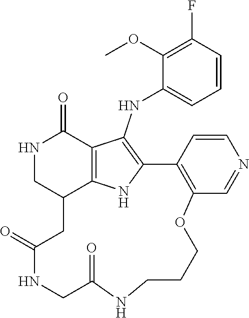 [SMILES: COc1c(F)cccc1Nc1c2[nH]c3c1C(=O)NCC3CC(=O)NCC(=O)NCCCOc1cnccc1-2] | 523.211 | 523.3 |
| 133 | 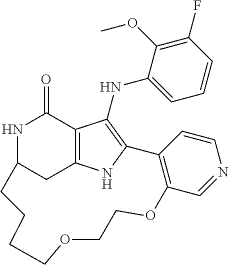 [SMILES: COc1c(F)cccc1Nc1c2[nH]c3c1C(=O)NC(CCCCOCCOc1cnccc1-2)C3] | 467.209 | 467.4 |
| 134 | 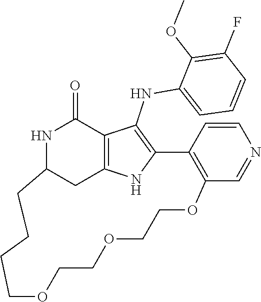 [SMILES: COc1c(F)cccc1Nc1c2[nH]c3c1C(=O)NC(CCCCOCCOCCOc1cnccc1-2)C3] | 511.236 | 511.3 |
| 135 | 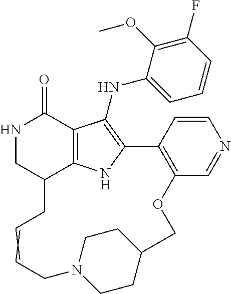 [SMILES: COc1c(F)cccc1Nc1c2[nH]c3c1C(=O)NCC3CC=CCN1CCC(CC1)COc1cnccc1-2] | 518.257 | 518.4 |
| 136 | 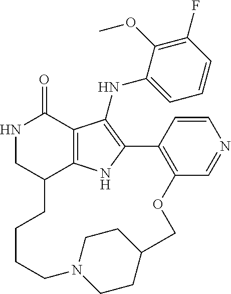 [SMILES: COc1c(F)cccc1Nc1c2[nH]c3c1C(=O)NCC3CCCCN1CCC(CC1)COc1cnccc1-2] | 520.272 | 520 |
| 137 | 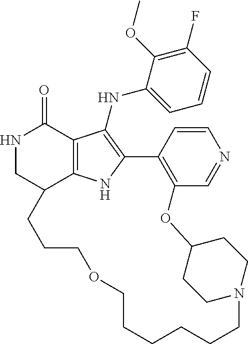 [SMILES: COc1c(F)cccc1Nc1c2[nH]c3c1C(=O)NCC3CCCOCCCCCCN1CCC(CC1)Oc1cnccc1-2] | 592.330 | 592.5 |
| 138 | 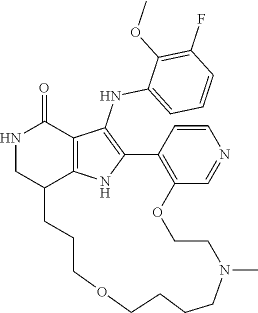 [SMILES: COc1c(F)cccc1Nc1c2[nH]c3c1C(=O)NCC3CCCOCCCCN(C)CCOc1cnccc1-2] | 538.283 | 538.4 |
| 139 | 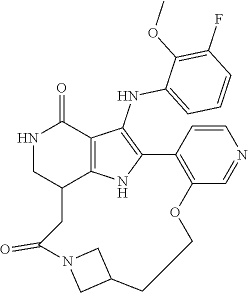 [SMILES: COc1c(F)cccc1Nc1c2[nH]c3c1C(=O)NCC3CC(=O)N1CC(CCOc3cnccc3-2)C1] | 492.205 | 492.3 |
| 140 | 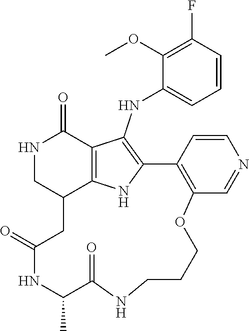 [SMILES: COc1c(F)cccc1Nc1c2[nH]c3c1C(=O)NCC3CC(=O)N[C@@H](C)C(=O)NCCCOc1cnccc1-2] | 537.226 | 537.3 |
| 141 | 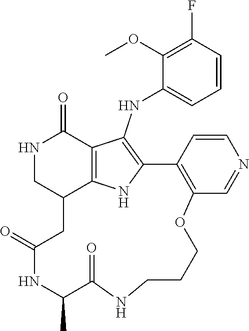 [SMILES: COc1c(F)cccc1Nc1c2[nH]c3c1C(=O)NCC3CC(=O)N[C@H](C)C(=O)NCCCOc1cnccc1-2] | 537.226 | 537.4 |
| 142 | 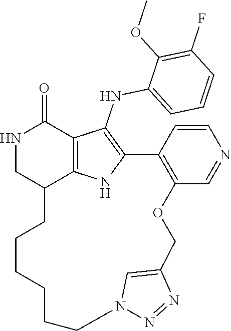 [SMILES: COc1c(F)cccc1Nc1c2[nH]c3c1C(=O)NCC3CCCCCCn1cc(nn1)COc1cnccc1-2] | 532.247 | 532.3 |
| 143 | 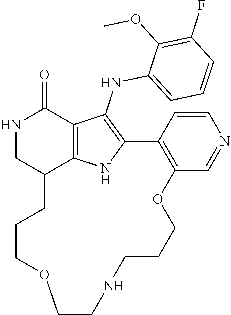 [SMILES: COc1c(F)cccc1Nc1c2[nH]c3c1C(=O)NCC3CCCOCCNCCCOc1cnccc1-2] | 510.252 | 510.4 |
| 144 | 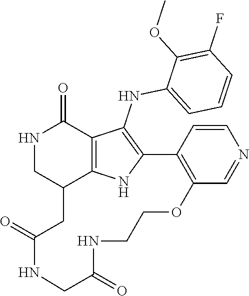 [SMILES: COc1c(F)cccc1Nc1c2[nH]c3c1C(=O)NCC3CC(=O)NCC(=O)NCCOc1cnccc1-2] | 509.195 | 509.1 |
| 145 | 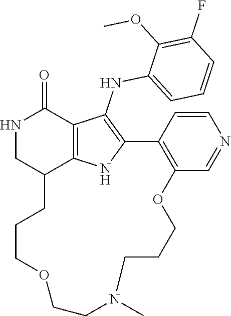 [SMILES: COc1c(F)cccc1Nc1c2[nH]c3c1C(=O)NCC3CCCOCCN(C)CCCOc1cnccc1-2] | 524.267 | 524.4 |
| 146 | 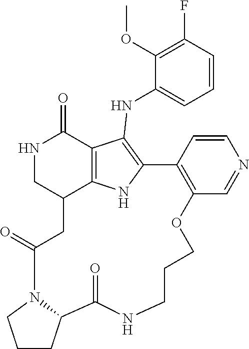 [SMILES: COc1c(F)cccc1Nc1c2[nH]c3c1C(=O)NCC3CC(=O)N1CCC[C@H]1C(=O)NCCCOc1cnccc1-2] | 563.242 | 563.3 |
| 147 | 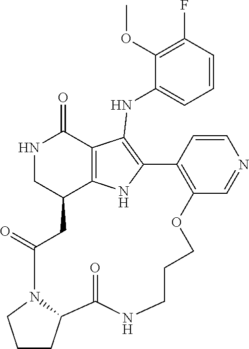 [SMILES: COc1c(F)cccc1Nc1c2[nH]c3c1C(=O)NC[C@@H]3CC(=O)N1CCC[C@H]1C(=O)NCCCOc1cnccc1-2] | 563.242 | 563.3 |
| 148 | 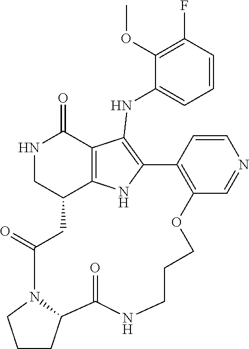 [SMILES: COc1c(F)cccc1Nc1c2[nH]c3c1C(=O)NC[C@H]3CC(=O)N1CCC[C@H]1C(=O)NCCCOc1cnccc1-2] | 563.242 | 563.5 |
| 149 | 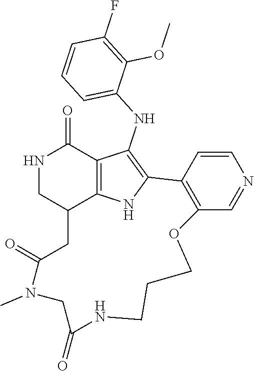 [SMILES: COc1c(F)cccc1Nc1c2[nH]c3c1C(=O)NCC3CC(=O)N(C)CC(=O)NCCCOc1cnccc1-2] | 537.226 | 537.3 |
| 150 | 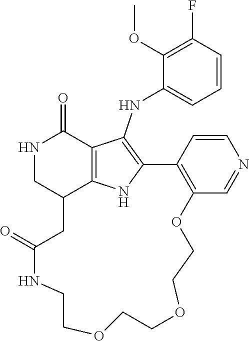 [SMILES: COc1c(F)cccc1Nc1c2[nH]c3c1C(=O)NCC3CC(=O)NCCOCCOCCOc1cnccc1-2] | 540.226 | 540.3 |
| 151 | 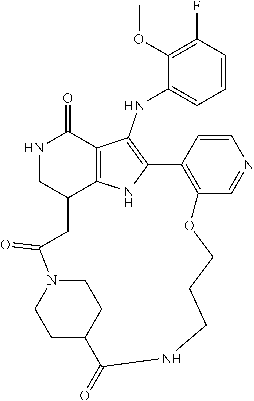 [SMILES: COc1c(F)cccc1Nc1c2[nH]c3c1C(=O)NCC3CC(=O)N1CCC(CC1)C(=O)NCCCOc1cnccc1-2] | 577.257 | 577.3 |
| 152 | 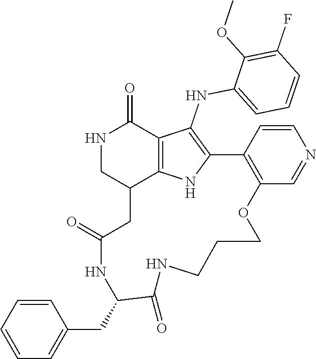 [SMILES: COc1c(F)cccc1Nc1c2[nH]c3c1C(=O)NCC3CC(=O)N[C@@H](Cc1ccccc1)C(=O)NCCCOc1cnccc1-2] | 613.257 | 613.4 |
| 153 | 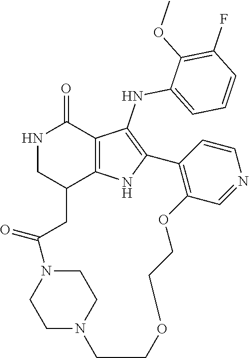 [SMILES: COc1c(F)cccc1Nc1c2[nH]c3c1C(=O)NCC3CC(=O)N1CCN(CCOCCOc3cnccc3-2)CC1] | 565.257 | 565 |
| 154 | 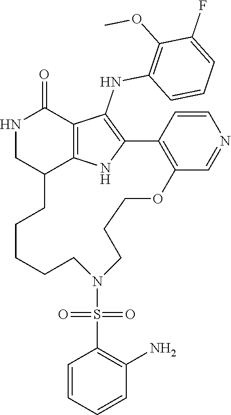 [SMILES: COc1c(F)cccc1Nc1c2[nH]c3c1C(=O)NCC3CCCCCN(S(=O)(=O)c1ccccc1N)CCCOc1cnccc1-2] | 649.261 | 649.2 |
| 155 | 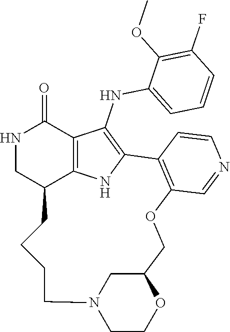 [SMILES: COc1c(F)cccc1Nc1c2[nH]c3c1C(=O)NC[C@@H]3CCCCN1CCO[C@H](COc3cnccc3-2)C1] | 522.252 | 522.3 |
| 156 | 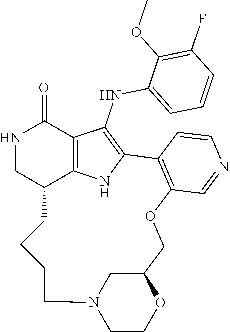 [SMILES: COc1c(F)cccc1Nc1c2[nH]c3c1C(=O)NC[C@H]3CCCCN1CCO[C@H](COc3cnccc3-2)C1] | 522.252 | 522.3 |
| 157 | 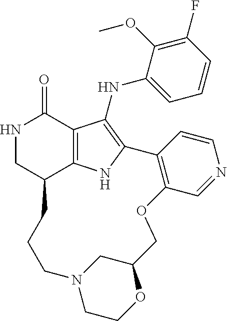 [SMILES: COc1c(F)cccc1Nc1c2[nH]c3c1C(=O)NC[C@@H]3CCCN1CCO[C@H](COc3cnccc3-2)C1] | 508.236 | 508.4 |
| 158 | 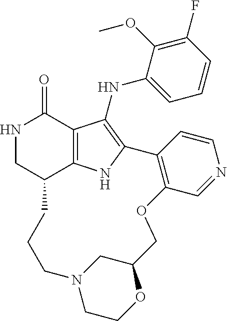 [SMILES: COc1c(F)cccc1Nc1c2[nH]c3c1C(=O)NC[C@H]3CCCN1CCO[C@H](COc3cnccc3-2)C1] | 508.236 | 508.3 |
| 159 | 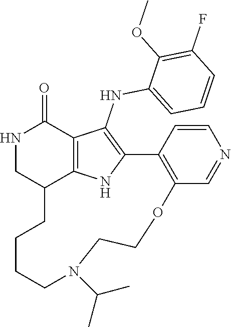 [SMILES: COc1c(F)cccc1Nc1c2[nH]c3c1C(=O)NCC3CCCCN(C(C)C)CCOc1cnccc1-2] | 508.272 | 508.3 |
| 160 | 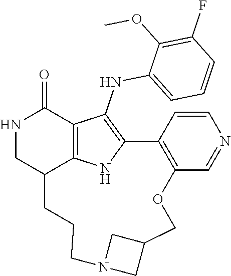 [SMILES: COc1c(F)cccc1Nc1c2[nH]c3c1C(=O)NCC3CCCN1CC(COc3cnccc3-2)C1] | 478.225 | 478.5 |
| 161 | 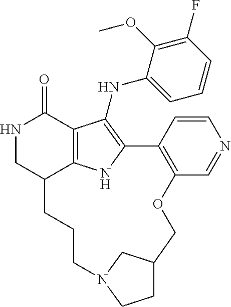 [SMILES: COc1c(F)cccc1Nc1c2[nH]c3c1C(=O)NCC3CCCN1CCC(COc3cnccc3-2)C1] | 492.241 | 492.5 |
| 162 | 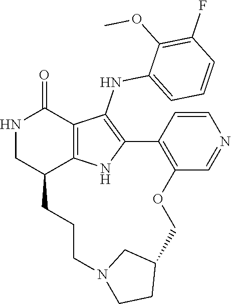 [SMILES: COc1c(F)cccc1Nc1c2[nH]c3c1C(=O)NC[C@@H]3CCCN1CC[C@@H](COc3cnccc3-2)C1] | 492.241 | 492.3 |
| 163 | 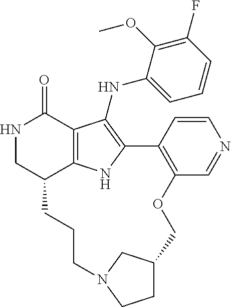 [SMILES: COc1c(F)cccc1Nc1c2[nH]c3c1C(=O)NC[C@H]3CCCN1CC[C@@H](COc3cnccc3-2)C1] | 492.241 | 492.3 |
| 164 | 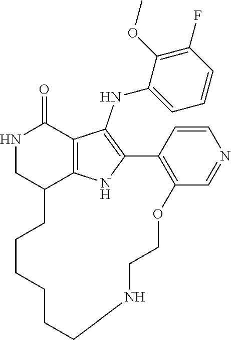 [SMILES: COc1c(F)cccc1Nc1c2[nH]c3c1C(=O)NCC3CCCCCCNCCOc1cnccc1-2] | 494.257 | 494.4 |
| 165 | 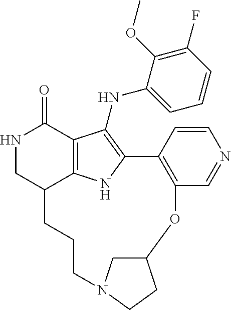 [SMILES: COc1c(F)cccc1Nc1c2[nH]c3c1C(=O)NCC3CCCN1CCC(C1)Oc1cnccc1-2] | 478.225 | 478.2 |
| 166 | 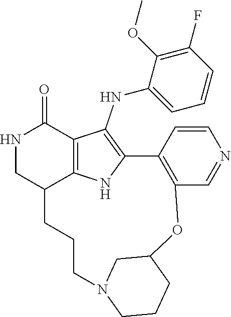 [SMILES: COc1c(F)cccc1Nc1c2[nH]c3c1C(=O)NCC3CCCN1CCCC(C1)Oc1cnccc1-2] | 492.241 | 492.5 |
| 167 | 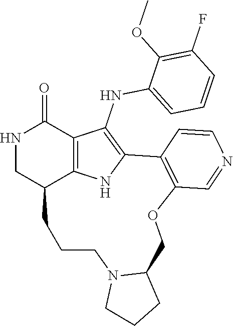 [SMILES: COc1c(F)cccc1Nc1c2[nH]c3c1C(=O)NC[C@@H]3CCCN1CCC[C@@H]1COc1cnccc1-2] | 492.241 | 492.3 |
| 168 | 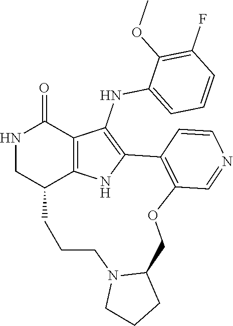 [SMILES: COc1c(F)cccc1Nc1c2[nH]c3c1C(=O)NC[C@H]3CCCN1CCC[C@@H]1COc1cnccc1-2] | 492.241 | 492.4 |
| 169 | 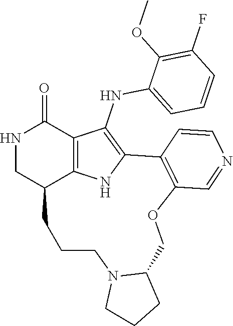 [SMILES: COc1c(F)cccc1Nc1c2[nH]c3c1C(=O)NC[C@@H]3CCCN1CCC[C@H]1COc1cnccc1-2] | 492.241 | 492.4 |
| 170 | 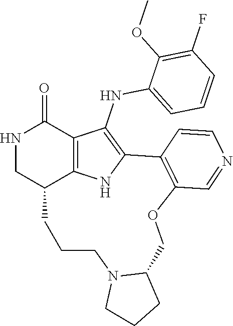 [SMILES: COc1c(F)cccc1Nc1c2[nH]c3c1C(=O)NC[C@H]3CCCN1CCC[C@H]1COc1cnccc1-2] | 492.241 | 492.3 |

### Synthesis of the Representative Examples of the Compound

### Example 1. (13E)-23-[(3-Fluoro-2-methoxyphenyl)amino]-8,11-dioxa-5,18,22-tniazatetracyclo[18.2.1.0^{2,7}.0¹⁶′2]tricosa-1(23),2,4,6,13,20-hexaen-19-one (Compound 99) and (13Z)-23-[(3-fluoro-2-methoxyphenyl)amino]-8,11-dioxa-5,18,22-triazatetracyclo[18.2.10^{2,7}.0^{16,21}]tricosa-1(23),2,4,6,13,20-hexaen-19-one (Compound 104)

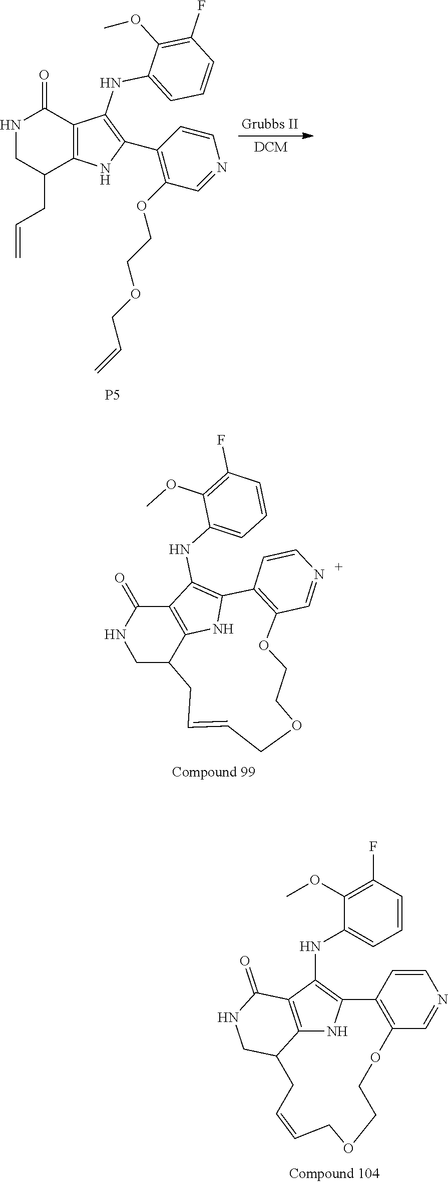

A solution of P5 (0.10 g, 0.20 mmol) in dry DCM (20 mL) was degassed by argon for 10 min. Grubbs catalyst 2nd generation (20 mg, 0.024 mmol) was added and reaction mixture was stirred at 50° C. for 1.5 h. Then the reaction mixture was concentrated in vacuo and purified by silica gel column chromatography (0-20% MeOH in DCM) to afford (E)-isomer Compound 99 (15 mg, 16%) as a yellow solid, and (Z)-isomer Compound 104 (4 mg, 4%) as a yellow solid. The E/Z isomers are difficult to separate so the mixture fraction of Compound 99 and Compound 104 (44 mg, 47%) was obtained as a yellow solid.

### Example 2. 23-[(3-Fluoro-2-methoxyphenyl)amino]-8,11-dioxa-5,18,22-triazatetracyclo[18.2.1.0^{2,7}.0^{16,21}]tricosa-1(23),2,4,6,20-pentaen-19-one (Compound 100)

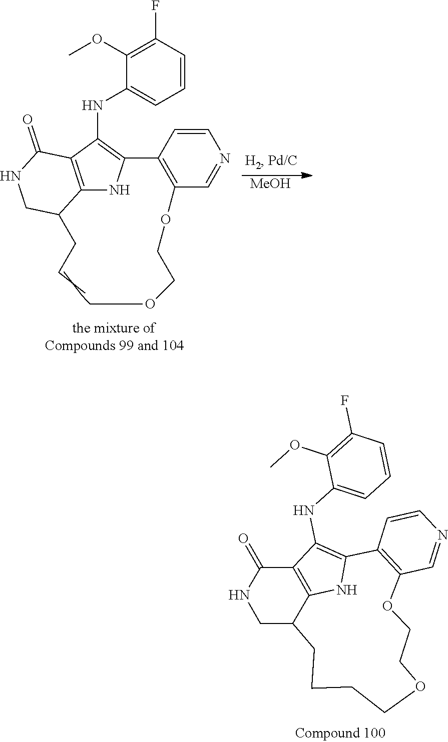 [SMILES: "MeOH" ; COc1c(F)cccc1Nc1c2[nH]c3c1C(=O)NCC3CC=CCOCCOc1cnccc1-2 ; COc1c(F)cccc1Nc1c2[nH]c3c1C(=O)NCC3CCCCOCCOc1cnccc1-2]

A mixture of E/Z isomers (Compound 99 and Compound 104, 20 mg, 0.043 mmol) and Pd/C (10 mg) in EtOH (1 mL) was stirred under H₂(g) balloon at rt for 5 h. The reaction was filtered and washed with EtOH. The filtrate was concentrated under reduced pressure. The residue was purified by silica gel column chromatography (0-20% MeOH in DCM) to afford Compound 100 (17 mg, 85%) as a yellow solid. ¹H NMR (400 MHz, CDCl₃), δ: 10.45 (s, 1H), 8.31 (s, 1H), 8.05 (d, J=5.1 Hz, 1H), 7.67 (s, 1H), 7.41 (d, J=5.1 Hz, 1H), 6.60 (td, J=8.2, 5.7 Hz, 1H), 6.48 (dd, J=10.1, J=8.2 Hz, 1H), 6.12 (d, J=8.2 Hz, 1H), 5.48 (s, 1H), 4.40-4.44 (m, 2H), 4.11 (s, 3H), 3.90-3.99 (m, 2H), 3.66-3.80 (m, 2H), 3.43-3.48 (m, 1H), 3.37 (t, J=11.6, 1H), 3.14-3.19 (m, 1H), 1.83-2.00 (m, 2H), 1.55-1.75 (m, 4H). LCMS (ESI) m/z: calc. for C₂₅ H₂₇FN₄O₄ 467.2095; found, 467.5 [M+H]⁺.

### Example 3. (16R)-23-[(3-Fluoro-2-methoxyphenyl)amino]-8,11-dioxa-5,18,22-triazatetracyclo[18.2.1.0^{2,7}.0^{16,21}]tricosa-1(23),2,4,6,20-pentaen-19-one (Compound 114) and (16S)-23-[(3-fluoro-2-methoxyphenyl)amino]-8,11-dioxa-5,18,22-triazatetracyclo[18.2102,70¹⁶,21tricosa-1(23),2,4,6,20-pentaen-19-one (Compound 115)

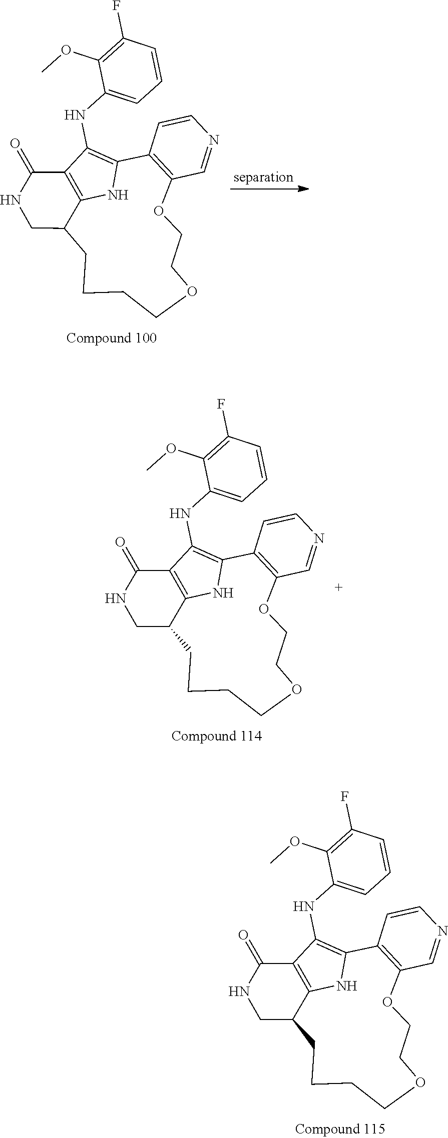 [SMILES: COc1c(F)cccc1Nc1c2[nH]c3c1C(=O)NCC3CCCCOCCOc1cnccc1-2 ; COc1c(F)cccc1Nc1c2[nH]c3c1C(=O)NC[C@H]3CCCCOCCOc1cnccc1-2 ; COc1c(F)cccc1Nc1c2[nH]c3c1C(=O)NC[C@@H]3CCCCOCCOc1cnccc1-2]

Compound 100 (15 mg, 0.032 mmol) was subjected to enantiomeric resolution on chiral column Phenomenex Lux® 5 μm Cellulose-4, LC Column 250×4.6 mm, mobile phase acetonitrile/formic acid 100:1, temperature 23° C., detector UV/CD 260 nm, flow rate 1 ml/min. The first enantiomer—Compound 114 (4 mg, 0.009 mmol, 27%) with tR 10.402 and the second enantiomer—Compound 115 (4 mg, 0.009 mmol, 27%) with tR 7.875 were obtained.

### Example 4. (19E)-28-[(3-Fluoro-2-methoxyphenyl)amino]-3,22-dioxa-6,11,15-triazapentacyclo[21.3.1^{10,13}.0^{4,9}.0^{12,17}]octacosa-1(27),4,6,8,10(28),12,19,23,25-nonaen-14-one (Compound 29)

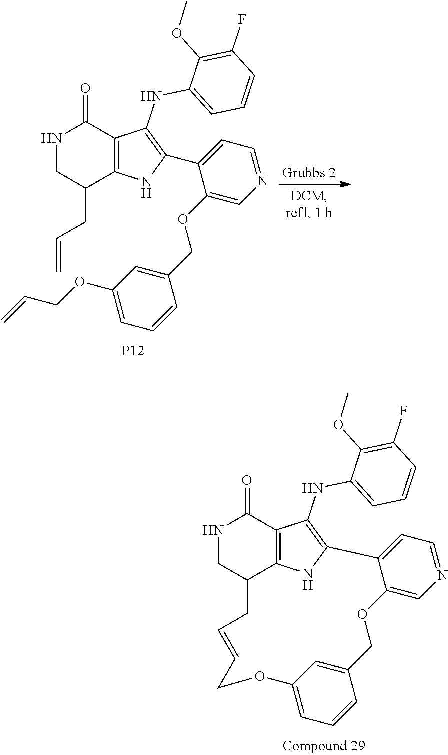

Hoveyda-Grubb's 2nd Generation Catalyst M720 (C₆₂₇) [1,3-bis(2,4,6-trimethylphenyl)imidazolidin-2-ylidene]-dichloro-[(2-propan-2-yloxyphenyl)methyldene]ruthenium (16 mg, 0.025 mmol, 0.1 eq) was added to a solution of P12 (140 mg, 0.25 mmol, 1 eq) in DCM (70 mL) under argon, then mixture was heated to reflux for 2 h. TLC (100% EtOAc). The product was purified by prep-HPLC to give Compound 29 (27 mg, 20%). ¹H NMR (400 MHz, DMSO), δ: 10.54 (s, 1H), 8.60 (s, 1H), 8.05-7.95 (m, 1H), 7.47 (s, 1H), 7.39-7.26 (m, 3H), 7.14-7.08 (m, 1H), 7.03-6.97 (m, 1H), 6.93-6.87 (m, 1H), 6.72-6.62 (m, 1H), 6.56-6.48 (m, 1H), 6.04-5.96 (m, 1H), 5.83-5.74 (m, 1H), 5.64-5.52 (m, 1H), 5.42-5.23 (m, 2H), 4.78-4.56 (m, 2H), 3.91 (s, 3H), 3.54-3.46 (m, 1H), 3.20-3.09 (m, 1H), 2.96-2.85 (m, 1H), 2.44-2.36 (m, 2H).

### Example 5. 28-[(3-Fluoro-2-methoxyphenyl)amino]-3,22-dioxa-6,11,15-triazapentacyclo[21.3.1.1^{10,13}.0^{4.9}.0^{12,17}]octacosa-1(27),4,6,8,10(28),12,23,25-octaen-14-one (Compound 2)

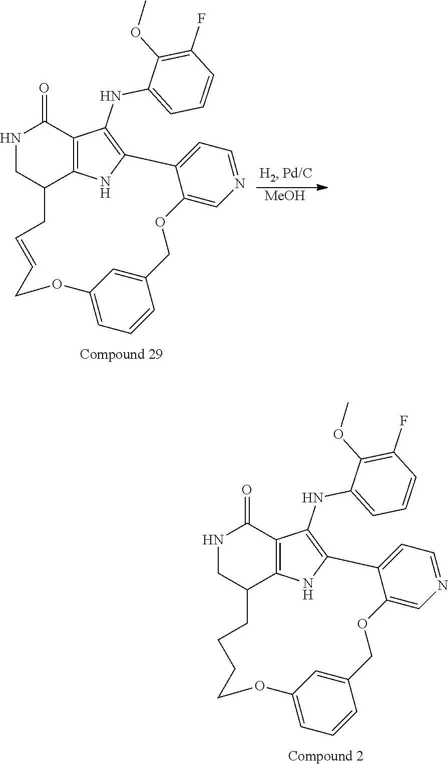 [SMILES: "MeOH" ; COc1c(F)cccc1Nc1c2[nH]c3c1C(=O)NCC3C/C=C/COc1cccc(c1)COc1cnccc1-2 ; COc1c(F)cccc1Nc1c2[nH]c3c1C(=O)NCC3CCCCOc1cccc(c1)COc1cnccc1-2]

Compound 29 (20 mg, 0.038 mmol) and Pd/C (10 mg) in EtOH (1 mL) was stirred under H₂(_{g}) balloon at rt for 5 h. The reaction was filtered and washed with EtOH. The filtrate was concentrated under reduced pressure. The residue was purified by silica gel column chromatography (0-20% MeOH in DCM) to afford Compound 2 (16 mg, 80%) as a solid.

### Example 6. 25-[(3-Fluoro-2-methoxyphenyl)amino]-7,8,9,10,14,15,18,18a,19,20-decahydro-6H-7,11-methano-22,24-methenodipyrido[3,4-b:4′,3′-f][1,5,15]oxadiazacycloicosine-12,21(13H,23H)-dione (Compound 116)

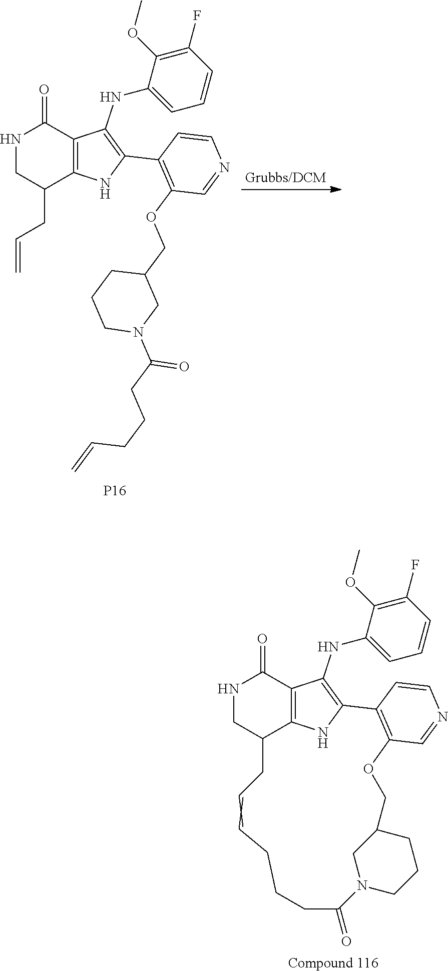

To a solution of 7-allyl-3-[(3-fluoro-2-methoxyphenyl)amino]-2-{3-[(1-hex-5-enoylpiperidin-3-yl)methoxy]pyridin-4-yl}-1,5,6,7-tetrahydro-4H-pyrrolo[3,2-c]pyridin-4-one P16 (120 mg, 0.2 mmol, 1.0 eq) in DCM (1.0 mL) Grubbs (5 mg) was added. The reaction mixture was reflux for 3 h, concentrated in vacuo. The residue after evaporation was subjected to preparative HPLC to afford the target compound 116 as a mixture of two isomers (Compound 117 and Compound 118). LCMS (ESI) m/z: 574.4 [M+H]⁺.

### Example 7. 25-[(3-Fluoro-2-methoxyphenyl)amino]-7,8,9,10,14,15,16,17,18,18a,19,20-dodecahydro-6H-7,11-methano-22,24-methenodipyrido[3,4-b:4′,3′-f][1,5,15]oxadiazacycloicosine-12,21(13H,23H)-dione (Compound 119)

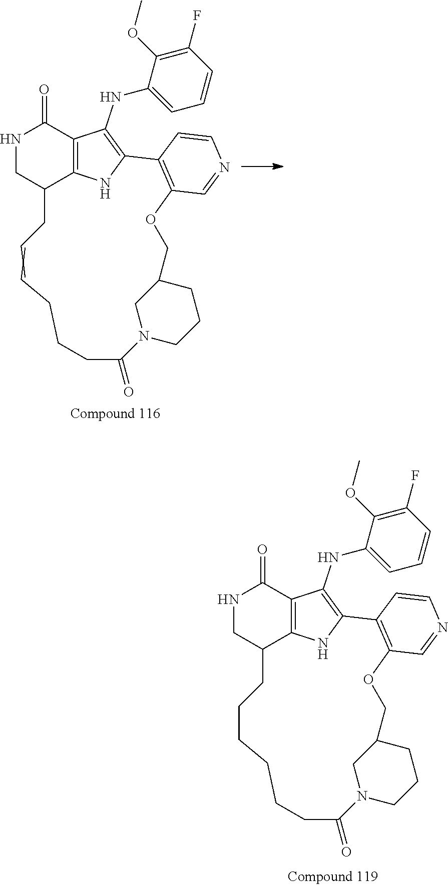

To compound 116 (10 mg, 0.02 mmol) in MeOH (1 mL) Pd/C (2 mg) was added and the mixture was stirred under H₂ atmosphere for 12 h. TLC indicated completion of the reaction. The catalyst was removed by filtration and the solution was evaporated to dryness to afford the target compound 119 (10 mg, 99%). LCMS (ESI) m/z: 576.6 [M+H]⁺.

### Example 8. 19-[(3-Fluoro-2-methoxyphenyl)amino]-8-methyl-6,7,8,9,10,11,12,12a,13,14-decahydro-16,18-methenodipyrido[3,4-i:4′,3′-m][1,4,11]oxadiazacyclotetradecin-15(17H)-one (Compound 127)

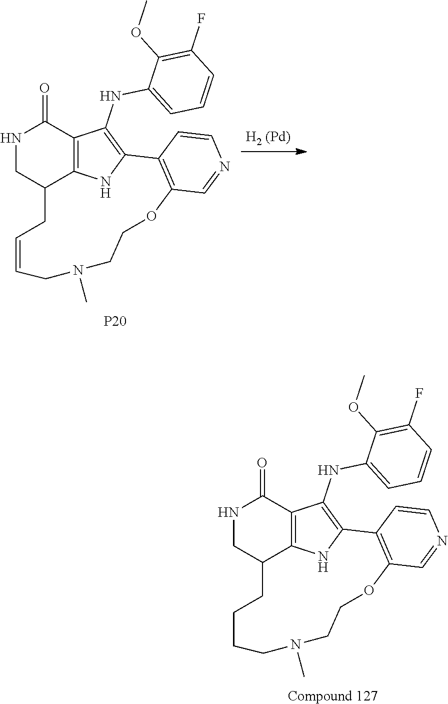

The mixture of P20 (80 mg, 0.17 mmol) in MeOH (1 mL) Pd/C (2 mg) was added and the mixture was stirred under H₂ atmosphere for 12 h. TLC indicated completion of the reaction. The catalyst was removed by filtration and the solution was evaporated to dryness to afford the target compound 127 (15 mg, 19%). LCMS (ESI⁺) m/z 481 [M+H]⁺. ¹H NMR (400 MHz, CDCl₃), δ: 11.04 (s, 1H), 8.31 (s, 1H), 8.05 (s, 1H), 7.55 (s, 1H), 7.45 (s, 1H), 6.66-6.52 (m, 1H), 6.55-6.40 (m, 1H), 6.09 (d, J=8.2 Hz, 1H), 5.28 (s, 1H), 4.48-4.27 (m, 2H), 4.11 (s, 3H), 3.52-3.40 (m, 1H), 3.38-3.25 (m, 1H), 3.20-2.90 (m, 2H), 2.84-2.60 (m, 2H), 2.61-2.46 (m, 1H), 2.20 (s, 3H), 1.95-1.46 (m, 9H).

### Example 9. 1³-((3-Fluoro-2-methoxyphenyl)amino)-1⁴,1⁵,1⁶,1⁷-tetrahydro-l¹H-3-oxa-1(2,7)-pyrrolo[3,2-c]pyridina-2(4,3)-pyridinacyclooctaphan-1⁴-one (Compound 101)

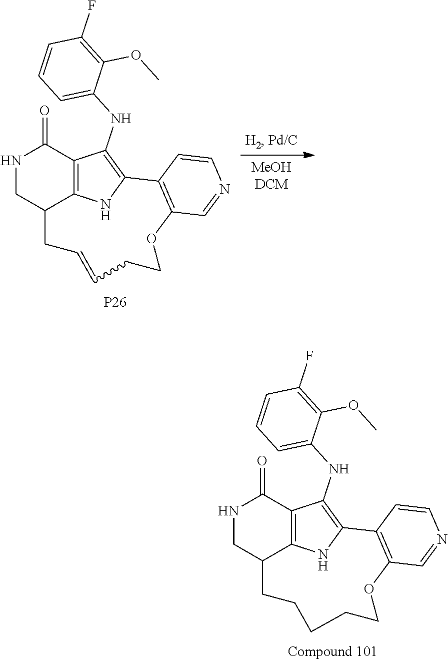

A mixture of trans/cis isomer P26 (100 mg, 0.23 mmol) and Pd/C (50 mg) in MeOH (10.0 mL) was stirred under H₂(g) balloon at rt for 48 h. The reaction was filtered through a pad of celite and washed with MeOH. The filtrate was concentrated under reduced pressure. The residue was puriied by C₁₈ column chromatography (10-100% MeOH in H₂O) to afford compound 101 (20 mg, 20%) as a yellow solid. ¹H NMR (400 MHz, CDCl₃), δ: 10.72 (br. s, 1H), 8.44 (s, 1H), 8.21 (d, J=5.2 Hz, 1H), 7.26-7.24 (m, 2H), 6.92 (s, 1H), 6.72-6.69 (m, 1H), 6.54-6.49 (m, 1H), 6.13 (d, J=8.2 Hz, 1H), 5.21 (d, J=3.8 Hz, 1H), 4.09 (s, 3H), 4.06-4.04 (m, 1H), 3.91 (dd, J=12.2, 5.0 Hz, 1H), 3.80-3.68 (m, 1H), 3.27-3.24 (m, 1H), 3.21-3.11 (m, 1H), 2.37-2.20 (m, 1H), 2.06-1.93 (m, 1H), 1.79-1.68 (m, 4H), 1.61-1.54 (m, 1H), 1.43-1.29 (m, 1H), 1.05-0.95 (m, 1H); LCMS (ESI) m/z calc. for C₂4H₂₅FN₄O₃ 436.19; found, 437.2 [M+H]⁺; HPLC purity: 99.76%, tR=15.141 min.

### Example 10. 23-[(3-Fluoro-2-methoxyphenyl)amino]-12-methyl-8-oxa-5,12,18,22-tetraazatetracyclo[18.2.1.0^{2,7}.0^{16,21}]tricosa-1(23),2,4,6,20-pentaen-19-one (Compounds 8) and 23-[(3-fluoro-2-methoxyphenyl)amino]-8-oxa-5,12,18,22-etraazatetracyclo[18.2.1.0^{2,7}.0^{16,21}]tricosa-1(23),2,4,6,20-pentaen-19-one (Compound 9)

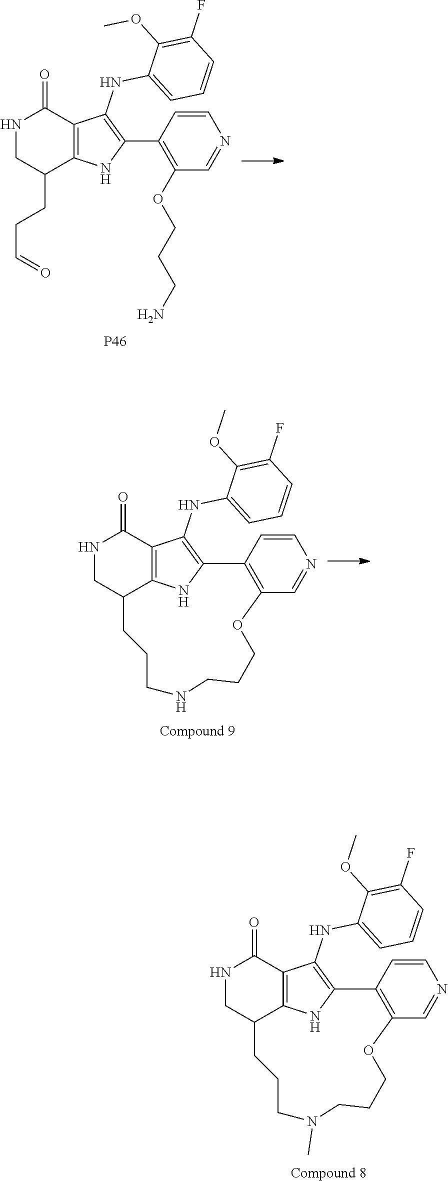

To a solution of crude P46 (55 mg, 1 eq) in DCE was added AcOH (45 mg, 15 eq), STAB (70 mg, 3 eq) and the mixture was stirred at rt for 10 h. Compound 9 was isolated. Then, if no need to isolate compound 9 25 μl (2 eq) of HCHO (37% aq.) was added, followed by additional 70 mg of STAB, the reaction mixture was stirred at rt for 12 h. To the reaction mixture was added sat. NaHCO₃ solution and organics was extracted with DCM, combined extract was washed with brine, dried over Na₂SO₄ and rotary evaporated to dryness. To the residue was added MeOH (5 ml) and NH₄0H (2.5 ml) and the mixture was stirred and 50° C. for 17 h. The reaction mixture was rotary evaporated to dryness. The residue was purified by HPLC, giving 18 mg of compound 8.

### Example 11. 21-[(3-Fluoro-2-methoxyphenyl)amino]-7,8,9,10,12,13,14,14a,15,16-decahydro-6H-7,11-methano-18,20-methenodipyrido[3,4-b:4′,3′-f][1,5,11]oxadiazacyclohexadecin-17(19H)-one (Compound 12)

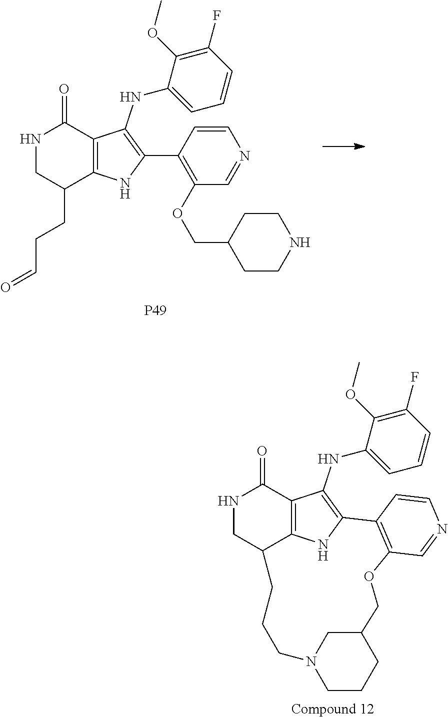

To a solution of P49 (250 mg, 0.47 mmol, 1 eq), DIPEA (0.25 mL, 1.4 mmol, 3 eq) in DCM (2 mL) STAB (610 mg, 2.8 mmol) was added. Then the reaction mixture was stirred at rt for 2 h. The reaction mixture was treated with aq. saturated solution of sodium bicarbonate to adjust pH=7, and extracted with DCM three times. The combined organic layers were dried over Na₂SO₄, filtered, and concentrated in vacuo. The residue after evaporation was subjected to HPLC to afford the target compounds 12 (29 mg). LCMS (ESI⁺) m/z 506 [M+H]⁺.

### Example 12. 25-[(3-Fluoro-2-methoxyphenyl)amino]-7,8,9,10,14,15,18,18a,19,20-decahydro-6H-7,11-methano-22,24-methenodipyrido[3,4-b:4′,3′-f][1,5,15]oxadiazacycloicosine-12,21(13H,23H)-dione (Compounds 117 and 118)

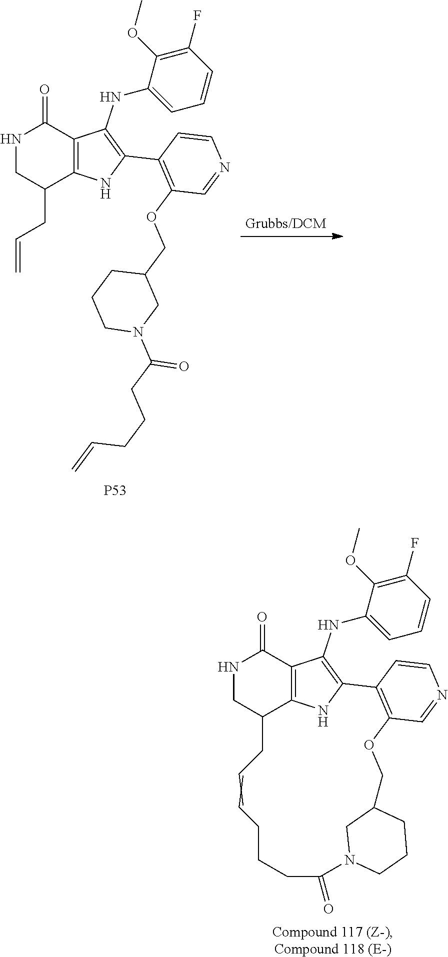

To a solution of P53 (120 mg, 0.2 mmol, 1.0 eq) in DCM (1.0 mL) Grubbs (5 mg) was added. The reaction mixture was reflux for 3 h, concentrated in vacuo. The residue after evaporation was subjected to HPLC to afford the target compounds 117 and 118.

### Example 13. 25-[(3-Fluoro-2-methoxyphenyl)amino]-7,8,9,10,14,15,16,17,18,18a,19,20-dodecahydro-6H-7,11-methano-22,24-methenodipyrido[3,4-b:4′,3′-f][1,5,15]oxadiazacycloicosine-12,21(13H,23H)-dione (Compound 119)

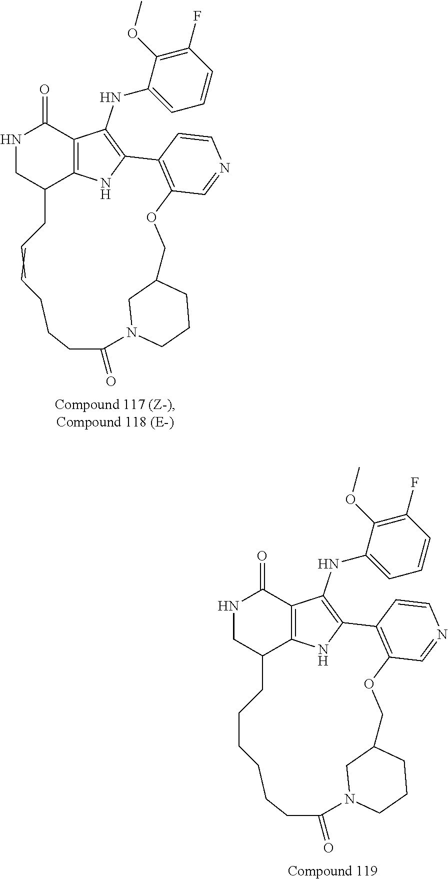

The mixture of compounds 117 and 118 (10 mg, 0.02 mmol) in MeOH (1 mL). Pd/C (2 mg) was added and the mixture was stirred under H₂ atmosphere for 12 h. TLC indicated completion of the reaction. The catalyst was removed by filtration and the solution was evaporated to dryness to afford the target compound 119 (10 mg, 99%). LCMS (ESI⁺) m/z 576 [M+H]⁺.

### Example 14. 21-[(3-Fluoro-2-methoxyphenyl)amino]-6,7,8,9,12,13,14,14a,15,16-decahydro-11H-7,10-ethano-18,20-methenodipyrido[3,4-b:4′,3′-f][1,5,12]oxadiazacyclohexadecin-17(19H)-one (Compound 136)

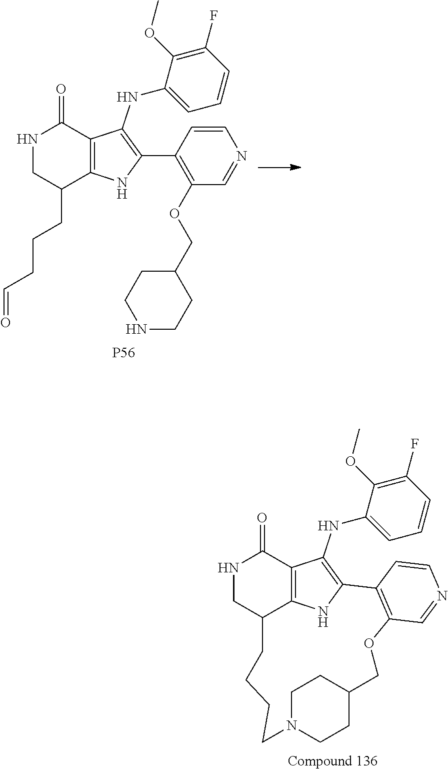

To solution of P56 (100 mg, 0.1 mmol, 1 eq), DIPEA (0.05 mL, 0.3 mmol, 3 eq) in DCM (2 mL) STAB (130 mg, 0.6 mmol) was added. Then the reaction mixture was stirred at rt for 2 h. Then reaction mixture was treated with aq. saturated solution of sodium bicarbonate to adjust pH=7, and extracted with DCM three times. The combined organic layers were dried over Na₂SO₄, filtered, and concentrated in vacuo. The residue after evaporation was subjected to HPLC to afford the target compound 136 (8 mg). LCMS (ESI⁺) m/z 520 [M+H]⁺.

### Example 15. 29-(3-Fluoro-2-methoxyanilino)-18,21-dioxa-1,6,10,15,24-pentaazapentacyclo[22.2.2.1^{8,11}0^{4,9}.0^{12,17}]nonacosa-8,11(29),12(17),13,15-pentaene-2,7-dione (Compound 153)

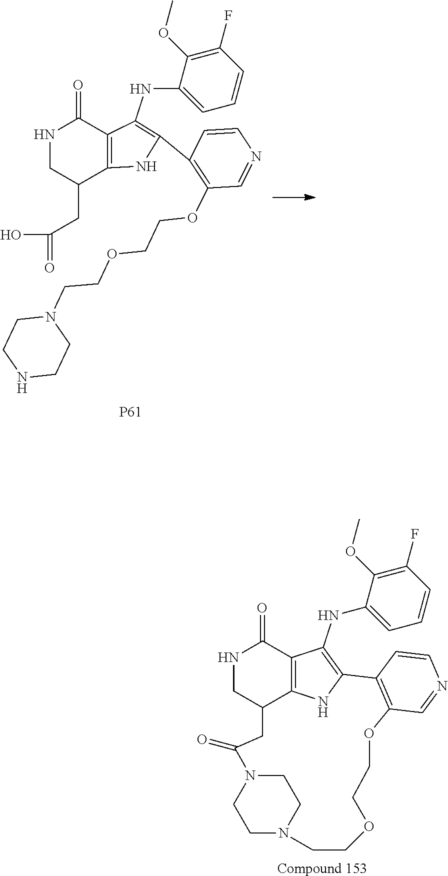

To solution of P61 (85 mg, 0.13 mmol, 1 eq) EDCI (80 mg, 3 eq) in DMF (3 mL) was added. Then the reaction mixture was stirred at rt for overnight. Then reaction mixture was treated with brine, extraction attempt from water was not successful. The concentrated in vacuo residue was purified by HPLC (also it can be purified by silica gel column chromatography with eluent DCM:MeOH 20%: Et₃N 1%) to give compound 153 (13 mg, 16%) as an orange solid. LCMS (ESI⁺) m/z 565 [M+H]⁺.

### Example 16. (7S)-22-[(3-Fluoro-2-methoxyphenyl)amino]-6,7,9,10,13,14,15,15a,16,17-decahydro-12H-7,11-methano-19,21-methenodipyrido[3,4-l:4′,3′-p][1,4,7,14]dioxadiazacycloheptadecin-18(20H)-one (Compounds 155 and 156)

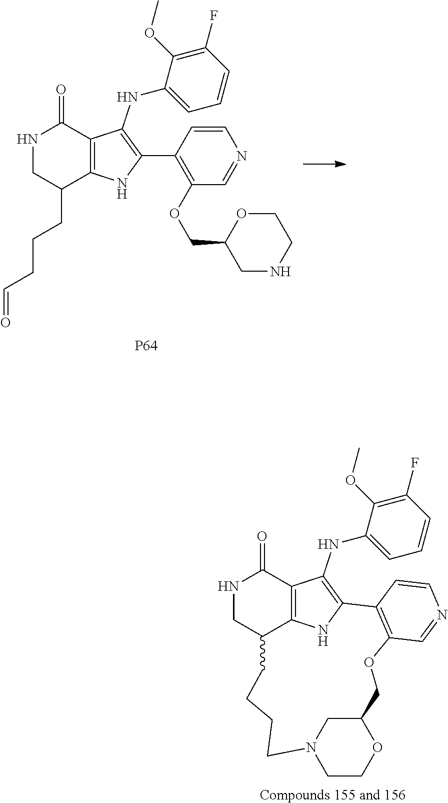

To a solution of P64 (100 mg, 0.1 mmol, 1 eq), DIPEA (0.05 mL, 0.3 mmol, 3 eq) in DCM (2 mL) STAB (130 mg, 0.6 mmol) was added. Then the reaction mixture was stirred at rt for 2 h. Then the reaction mixture was treated with aq. sat. solution of sodium bicarbonate to adjust pH=7, and extracted with DCM three times. The combined organic layers were dried over Na₂SO₄, filtered, and concentrated in vacuo. The residue after evaporation was subjected to HPLC to afford the target compounds 155 (12 mg) and 156 (13 mg). Compound 155: ¹H NMR (400 MHz, DMSO-d₆), δ: 11.45 (s, 1H), 9.71 (s, 1H), 8.57 (s, 1H), 8.20 (d, J=5.4 Hz, 1H), 7.49 (s, 1H), 7.32 (d, J=5.4 Hz, 1H), 7.19 (s, 1H), 6.63 (td, J=8.3, 6.0 Hz, 1H), 6.55-6.36 (m, 1H), 6.00 (d, J=8.2 Hz, 1H), 4.56-4.42 (m, 1H), 4.12 (q, J=12.6, 11.7 Hz, 4H), 3.96-3.77 (m, 4H), 3.73 (d, J=12.5 Hz, 1H), 3.57-2.96 (m, 8H), 2.25-1.99 (m, 1H), 1.99-1.73 (m, 2H), 1.73-1.38 (m, 2H). LCMS (ESI⁺) m/z 522.3 [M+H]⁺. Compound 156: ¹H NMR (400 MHz, DMSO-d₆), δ: 11.39 (s, 1H), 10.19 (s, 1H), 8.63 (s, 1H), 8.23 (d, J=5.8 Hz, 1H), 7.75 (s, 1H), 7.43 (d, J=5.9 Hz, 1H), 7.28 (s, 1H), 6.80-6.59 (m, 1H), 6.59-6.40 (m, 1H), 6.02 (d, J=8.3 Hz, 1H), 4.66 (d, J=6.0 Hz, 1H), 4.25 (d, J=8.1 Hz, 2H), 4.18-4.00 (m, 1H), 3.97-3.69 (m, 4H), 3.60-2.97 (m, 8H), 1.95-1.31 (m, 7H). LCMS (ESI⁺) m/z 522.3 [M+H]⁺.

### Example 17. (7S)-21-[(3-Fluoro-2-methoxyphenyl)amino]-6,7,9,10,12,13,14,14a,15,16-decahydro-7,11-methano-18,20-methenodipyrido[3,4-k:4′,3′-o][1,4,7,13]dioxadiazacyclohexadecin-17(19H)-one (Compounds 157 and 158)

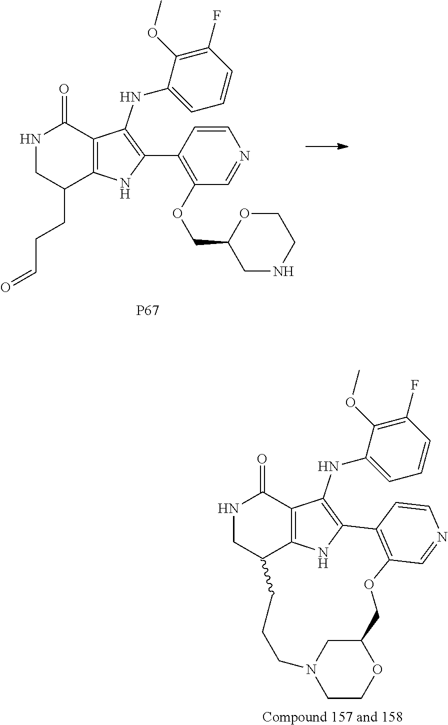 [SMILES: "P67" ; COc1c(F)cccc1Nc1c(-c2ccncc2OC[C@@H]2CNCCO2)[nH]c2c1C(=O)NCC2CCC=O ; COc1c(F)cccc1Nc1c2[nH]c3c1C(=O)NCC3CCCN1CCO[C@H](COc3cnccc3-2)C1 ; "Compound 157 and 158"]

To a solution of P67 (300 mg, 0.6 mmol, 1 eq), DIPEA (0.3 mL, 1.7 mmol, 3 eq) in DCM (2 mL) STAB (720 mg, 3.4 mmol) was added. Then the reaction mixture was stirred at rt for 2 h. Then reaction mixture was treated with aq. sat. solution of sodium bicarbonate to adjust pH=7, and extracted with DCM three times. The combined organic layers were dried over Na₂SO₄, filtered, and concentrated in vacuo. The residue after evaporation was subjected to HPLC to afford the target compounds 157 (48 mg) and 158 (18 mg). Compound 157: ¹H NMR (400 MHz, DMSO-d₆), δ: 11.57 (s, 1H), 10.22 (s, 1H), 8.59 (s, 1H), 8.23 (d, J=5.4 Hz, 1H), 7.62 (s, 1H), 7.34 (d, J=5.5 Hz, 1H), 7.21 (s, 1H), 6.78-6.56 (m, 1H), 6.56-6.39 (m, 1H), 5.94 (d, J=8.3 Hz, 1H), 4.58 (d, J=6.4 Hz, 1H), 4.23 (s, 1H), 4.18-3.98 (m, 2H), 3.88 (s, 3H), 3.65-2.93 (m, 8H), 2.01-1.68 (m, 5H), 1.56-1.32 (m, 1H). LCMS (ESI⁺) m/z 508.4 [M+H]⁺. Compound 158: ¹H NMR (400 MHz, DMSO-d₆), δ: 11.20 (s, 1H), 8.69-8.38 (m, 1H), 8.22 (s, 1H), 7.97-7.62 (m, 1H), 7.57-7.19 (m, 2H), 6.81-6.46 (m, 2H), 5.98 (s, 1H), 4.19 (s, 2H), 3.91 (s, 3H), 3.76 (s, 1H), 3.58 (s, 1H), 3.35-2.72 (m, 1OH), 2.01-1.46 (m, 4H). LCMS (ESI⁺) m/z 508.3 [M+H]⁺.

### Example 18. 19-[(3-Fluoro-2-methoxyphenyl)amino]-7,8,10,11,12,12a,13,14-octahydro-6H-7,9-methano-16,18-methenodipyrido[3,4-b:4′,3′-f][1,5,11]oxadiazacyclotetradecin-15(17H)-one (Compound 160)

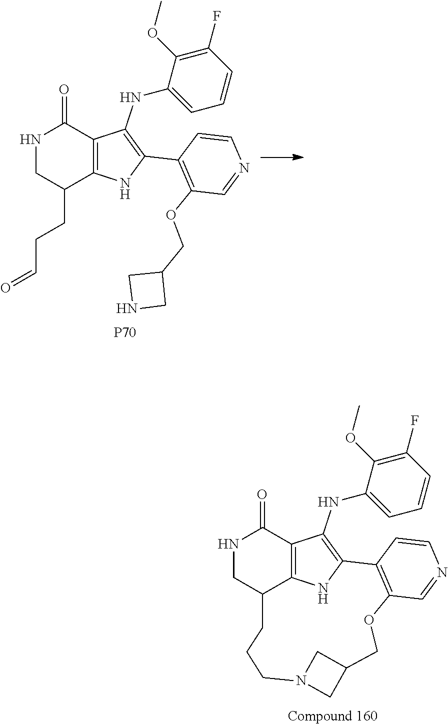

To solution of P70 (230 mg, 0.38 mmol, 1 eq), DIPEA (0.32 mL, 1.9 mmol, 5 eq) in DCM (2 mL) STAB (240 mg, 1.1 mmol) was added. Then the reaction mixture was stirred at rt for 2 h. Then the reaction mixture was treated with aq. sat. solution of sodium bicarbonate to adjust pH=7, and extracted with DCM three times. The combined organic layers were dried over Na₂SO₄, filtered, and concentrated in vacuo. The residue was purified by silica gel column chromatography (DCM:MeOH 0-5%) to give compound 160 (64 mg, 36%) as a yellow oil. LCMS (ESI⁺) m/z 478.5 [M+H]⁺.

### Example 19. 20-[(3-Fluoro-2-methoxyphenyl)amino]-6,7,8,9,11,12,13,13a,14,15-decahydro-7,10-methano-17,19-methenodipyrido[3,4-b:4′,3′-f][1,5,11]oxadiazacyclopentadecin-16(18H)-one (Compounds 162 and 163)

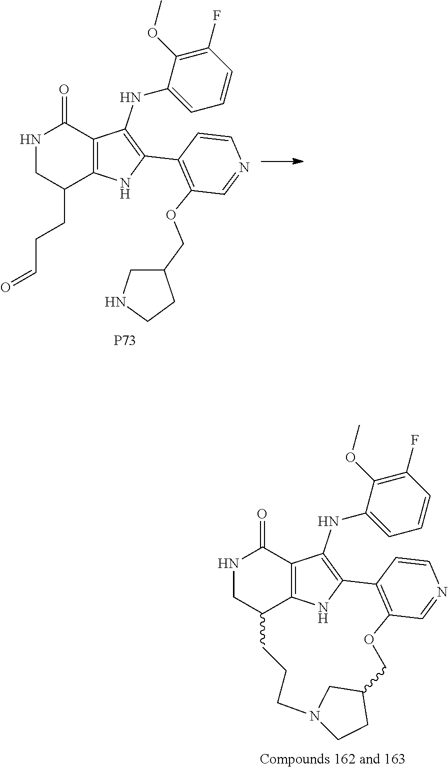 [SMILES: "P73" ; COc1c(F)cccc1Nc1c(-c2ccncc2OCC2CCNC2)[nH]c2c1C(=O)NCC2CCC=O ; COc1c(F)cccc1Nc1c2[nH]c3c1C(=O)NCC3CCCN1CCC(COc3cnccc3-2)C1 ; "Compounds 162 and 163"]

To solution of P73 (150 mg, 0.6 mmol, 1 eq), DIPEA (0.13 mL, 0.74 mmol, 3 eq) in DCM (2 mL) STAB (160 mg, 0.74 mmol) was added. Then the reaction mixture was stirred at rt for 2 h. Then the reaction mixture was treated with aq. sat. solution of sodium bicarbonate to adjust pH=7, and extracted with DCM three times. The combined organic layers were dried over Na₂SO₄, filtered, and concentrated in vacuo. The residue was purified by silica gel column chromatography (DCM:MeOH 0-10%) to give the target compounds 162 (4 mg) and 163 (5 mg).

### Example 20. (12aR)-20-[(3-fluoro-2-methoxyphenyl)amino]-1,2,12a,13,14,15,17,18,19,19a-decahydro-12H-4,6-methenodipyrido[3,4-h:4′,3′-l]pyrrolo[2,1-c][1,4,10]oxadiazacyclotridecin-3(5H)-one (Compounds 167 and 168)

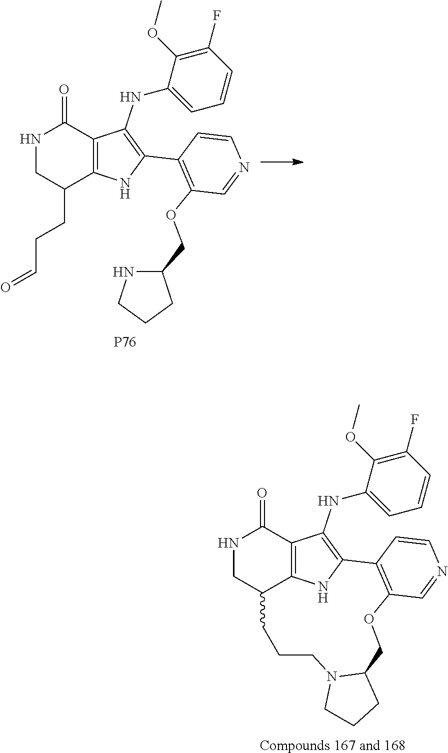 [SMILES: "P76" ; COc1c(F)cccc1Nc1c(-c2ccncc2OC[C@H]2CCCN2)[nH]c2c1C(=O)NCC2CCC=O ; COc1c(F)cccc1Nc1c2[nH]c3c1C(=O)NCC3CCCN1CCC[C@@H]1COc1cnccc1-2 ; "Compounds 167 and 168"]

To solution of P76 (350 mg, 0.6 mmol, 1 eq), DIPEA (0.32 mL, 1.9 mmol, 3 eq) in DCM (2 mL) STAB (440 mg, 1.8 mmol) was added. Then the reaction mixture was stirred at rt for 2 h. Then reaction mixture was treated with aq. sat. solution of sodium bicarbonate to adjust pH=7, and extracted with DCM three times. The combined organic layers were dried over Na₂SO₄, filtered, and concentrated in vacuo. The residue after evaporation was subjected to HPLC to afford the target compounds 167 (8 mg) and 168 (79 mg). Compound 168: ¹H NMR (400 MHz, DMSO-d₆), δ: 11.30 (s, 1H), 9.62 (s, 1H), 8.48 (s, 1H), 8.29 (d, J=5.1 Hz, 1H), 7.53-7.24 (m, 3H), 6.99 (s, 2H), 6.66 (td, J=8.3, 6.1 Hz, 1H), 6.52-6.34 (m, 1H), 6.06 (d, J=8.2 Hz, 1H), 4.57 (dd, J=10.7, 3.7 Hz, 1H), 4.28 (t, J=10.8 Hz, 1H), 3.81 (s, 3H), 3.60 (dd, J=12.5, 5.4 Hz, 1H), 3.25-2.87 (m, 5H), 2.35-1.81 (m, 7H). LCMS (ESI⁺) m/z 492.4 [M+H]⁺.

### Example 21. (12aS)-20-[(3-Fluoro-2-methoxyphenyl)amino]-1,2,12a,13,14,15,17,18,19,19a-decahydro-12H-4,6-methenodipyrido[3,4-h:4′,3′-l]pyrrolo[2,1-c][1,4,10]oxadiazacyclotridecin-3(5H)-one (Compounds 169 and 170)

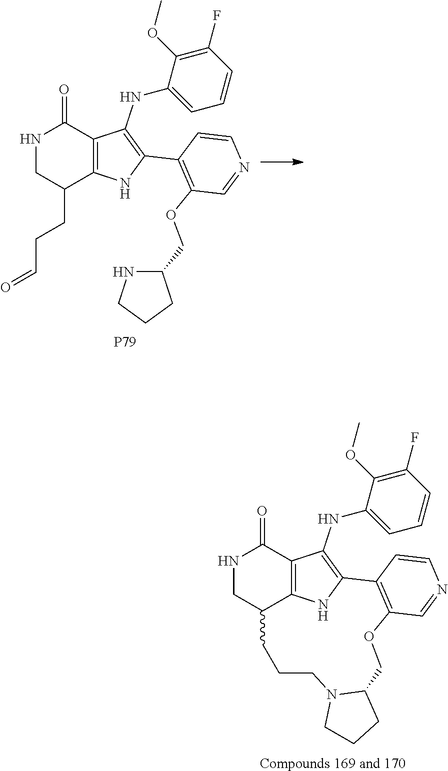 [SMILES: "P79" ; COc1c(F)cccc1Nc1c(-c2ccncc2OC[C@@H]2CCCN2)[nH]c2c1C(=O)NCC2CCC=O ; COc1c(F)cccc1Nc1c2[nH]c3c1C(=O)NCC3CCCN1CCC[C@H]1COc1cnccc1-2 ; "Compounds 169 and 170"]

To solution of P79 (370 mg, 0.6 mmol, 1 eq), DIPEA (0.32 mL, 1.9 mmol, 3 eq) in DCM (2 mL) STAB (440 mg, 1.8 mmol) was added. Then the reaction mixture was stirred at rt for 2 h. Then reaction mixture was treated with aq. sat. solution of sodium bicarbonate to adjust pH=7, and extracted with DCM three times. The combined organic layers were dried over Na₂SO₄, filtered, and concentrated in vacuo. The residue after evaporation was subjected to HPLC to afford the target compounds 169 (28 mg) and 170 (32 mg). Compound 169: ¹H NMR (400 MHz, DMSO-d₆), δ: 11.59 (s, 1H), 9.68 (s, 1H), 8.51 (s, 1H), 8.31 (d, J=5.5 Hz, 1H), 7.56-7.29 (m, 2H), 7.23-6.96 (m, 2H), 6.70 (td, J=8.2, 5.9 Hz, 1H), 6.54 (dd, J=10.7, 8.5 Hz, 1H), 6.02 (d, J=8.2 Hz, 1H), 4.90-4.57 (m, 1H), 4.27-4.06 (m, 1H), 3.89 (s, 3H), 3.67 (dd, J=12.6, 4.7 Hz, 1H), 3.55-3.22 (m, 3H), 3.22-2.81 (m, 3H), 2.40-2.24 (m, 1H), 2.14-1.78 (m, 3H), 1.78-1.40 (m, 3H). LCMS (ESI⁺) m/z 492.4 [M+H]⁺. Compound 170: ¹H NMR (400 MHz, DMSO-d₆), δ: 11.38 (s, 1H), 9.78 (s, 1H), 8.48 (s, 1H), 8.29 (d, J=5.2 Hz, 1H), 7.36 (d, J=5.2 Hz, 1H), 7.00 (d, J=3.5 Hz, 2H), 6.66 (td, J=8.3, 6.0 Hz, 1H), 6.53-6.32 (m, 1H), 6.06 (d, J=8.3 Hz, 1H), 4.57 (dd, J=10.7, 3.6 Hz, 1H), 4.37-4.24 (m, 2H), 3.81 (s, 3H), 3.78-3.65 (m, 1H), 3.65-3.51 (m, 1H), 3.30-2.79 (m, 4H), 2.32-1.66 (m, 7H), 1.64-1.38 (m, 1H). LCMS (ESI⁺) m/z 492.3 [M+H]⁺.

Biological Assays

### Example A. Primary Assay Used to Determine Potency of EGFR (A767-S768insTLA) enzymatic activity Inhibition

Compound activity was determined using recombinant EGFR (A767-S768insTLA) protein (SignalChem, #E10-132CG-10) and Poly(Glu, Tyr) substrate (Sigma-Aldrich, #P0275) in an in vitro enzymatic reaction. The enzymatic assay used to determine activity was a Luminescence assay using a Microplate Reader ClarioStar Plus. The enzymatic reaction was carried out in assay buffer (40 mM TRIS-HCl pH 7.4-7.6, 20 mM MgCl₂, 2.5 mM MnCl₂, 0.05 mM DTT, 0.1 mg/ml BSA). The compounds were dispensed on a 384 well Diamond Well Plate (Axigen, Cat #P-384-120SQ-C—S) using the Biomek FX liquid handling system at 80× solutions of compounds in DMSO. 2× Protein-Substrate mix (final concentration 2.5 nM of EGFR (A767-S768insTLA) and 100 ng/μl of Poly(Glu, Tyr) was prepared in 1× Assay buffer and 4 μl of mixture per well was added into 384w white Reaction plate with NBS (Coming, Cat #4513). 4 μl of Poly(Glu, Tyr) substrate w/o EGFR in 1× buffer was used for negative control. Plates were centrifuged for 1 min at 200 g. Next step the Compounds were added to Reaction plate using Biomek station via following steps: 1 μl of 80× compounds (in DMSO) were mixed thoroughly with 39 μl of 2×10 μM ATP in Assay Buffer, then 4 μl of this mixture was added to Reaction plate with 4 μl of Protein-Substrate mix. Plates were centrifuged for 1 min at 200 g and incubated for 1 h at rt. Next 4 μL of ADP-Glo reagent (Promega, ADP-Glo™ Kinase Assay, Cat #V9102) per well was added. Plates were incubated for 30 min at rt. Then 8 μL of Kinase detection reagent (Promega, ADP-Glo™ Kinase Assay, Cat #V9102) per well was added and the Luminescence was measured using Microplate Reader. The % inhibition was then used to calculate the IC₅₀ values.

### Example B. Primary Assay Used to Determine Potency of EGFR (D770_N771insNPG) Enzymatic Activity Inhibition

Compounds activity was determined using recombinant EGFR (D770 N771insNPG) protein (SignalChem, #E10-132GG-10) and Poly(Glu, Tyr) substrate (Sigma-Aldrich, #P0275) in an in vitro enzymatic reaction. The enzymatic assay used to determine activity was a Luminescence assay using a Microplate Reader ClarioStar Plus. The enzymatic reaction was carried out in assay buffer (40 mM TRIS-HCl pH 7.4-7.6, 20 mM MgCl₂, 2.5 mM MnCl₂, 0.05 mM DTT, 0.1 mg/ml BSA). The compounds were dispensed on a 384 well Diamond Well Plate (Axigen, Cat #P-384-120SQ-C—S) using the Biomek FX liquid handling system at 80× solutions of compounds in DMSO. 2× Protein-Substrate mix (final concentration 1.5 nM of EGFR (D770_N771insNPG) and 50 ng/μl of Poly(Glu, Tyr)) was prepared in 1× Assay buffer and 4p1 of mixture per well was added into 384w white Reaction plate with NBS (Coming, Cat #4513). 4 μl of Poly(Glu, Tyr) substrate w/o EGFR in 1× buffer was used for negative control. Plates were centrifuged for 1 min at 200 g. Next step the Compounds were added to Reaction plate using Biomek station via following steps: 1 μl of 80× compounds (in DMSO) were mixed thoroughly with 39 μl of 2× 10 μM ATP in Assay Buffer, then 4 μl of this mixture was added to Reaction plate with 4 μl of Protein-Substrate mix. Plates were centrifuged for 1 min at 200 g and incubated for 1 h at rt. Next 4 μL of ADP-Glo reagent (Promega, ADP-Glo™ Kinase Assay, Cat #V9102) per well was added. Plates were incubated for 30 min at rt. Then 8 μL of Kinase detection reagent (Promega, ADP-Glo™ Kinase Assay, Cat #V9102) per well was added and the Luminescence was measured using Microplate Reader. The % inhibition was then used to calculate the EC₅₀ values. The values of EC₅₀ shown as a letters A-E, where: A<0.001 μM; 0.001 μM<B<0.005 μM; 0.005 μM<C<0.01 μM; 0.01<D<0.05 μM; E>0.05.

TABLE B
Potency of EGFR (D770_N771insNPG)
enzymatic activity Inhibition

| | EC₅₀, |
|---|---|
| Compound # | μM* |
| 99 | B |
| 100 | B |
| 104 | A |
| 114 | B |
| 115 | B |

*EC₅₀: Half maximal effective concentration: the concentration of a compound which induces a response halfway between the baseline and maximum after a specific exposure time;
EC₅₀: A ≤ 0.001 μM;
0.001 μM < B ≤ 0.005 μM;
0.005 μM < C ≤ 0.01 μM;
0.01 < D ≤ 0.05 μM;
E > 0.05

### Example C. Primary Assay Used to Determine Potency of EGFR (T790M L⁸⁵⁸R) Enzymatic Activity Inhibition

Compound activity was determined using recombinant EGFR (T790M L⁸⁵⁸R) protein (SignalChem, #E10-122DG-10) and Poly(Glu, Tyr) substrate (Sigma-Aldrich, #P0275) in an in vitro enzymatic reaction. The enzymatic assay used to determine activity was a Luminescence assay using a Microplate Reader ClarioStar Plus. The enzymatic reaction was carried out in assay buffer (40 mM TRIS-HCl pH 7.4-7.6, 20 mM MgCl₂, 2.5 mM MnCl₂, 0.05 mM DTT, 0.1 mg/ml BSA). The compounds were dispensed on a 384 well Diamond Well Plate (Axigen, Cat #P-384-120SQ-C—S) using the Biomek FX liquid handling system at 80× solutions of compounds in DMSO. 2× Protein-Substrate mix (final concentration 0.7 nM of EGFR (T790M L⁸⁵⁸R) and 100 ng/μl of Poly(Glu, Tyr)) was prepared in 1× Assay buffer and 4 μl of mixture per well was added into 384w white Reaction plate with NBS (Coming, Cat #4513). 4 μl of Poly(Glu, Tyr) substrate w/o EGFR in 1× buffer was used for negative control. Plates were centrifuged for 1 min at 200 g.

Next step the Compounds were added to Reaction plate using Biomek station via following steps: 1 μl of 80× compounds (in DMSO) were mixed thoroughly with 39 μl of 2× 10 μM ATP in Assay Buffer, then 4 μl of this mixture was added to Reaction plate with 4 μl of Protein-Substrate mix.

Plates were centrifuged for 1 min at 200 g and incubated for 1 h at rt. Next 4 μL of ADP-Glo reagent (Promega, ADP-Glo™ Kinase Assay, Cat #V9102) per well was added. Plates were incubated for 30 min at rt. Then 8 μL of Kinase detection reagent (Promega, ADP-Glo™ Kinase Assay, Cat #V9102) per well was added and the Luminescence was measured using Microplate Reader. The % inhibition was then used to calculate the IC₅₀ values.

### Example D. Primary Assay Used to Determine Potency of EGFR enzymatic activity Inhibition (WT)

Compound activity was determined using recombinant EGFR protein (SignalChem, #E10-112G-10) and Poly(Glu, Tyr) substrate (Sigma-Aldrich, #P0275) in an in vitro enzymatic reaction. The enzymatic assay used to determine activity was a Luminescence assay using a Microplate Reader ClarioStar Plus. The enzymatic reaction was carried out in assay buffer (40 mM TRIS-HCl pH 7.4-7.6, 20 mM MgCl₂, 2.5 mM MnCl₂, 0.05 mM DTT, 0.1 mg/ml BSA). The compounds were dispensed on a 384 well Diamond Well Plate (Axigen, Cat #P-384-120SQ-C—S) using the Biomek FX liquid handling system at 80× solutions of compounds in DMSO. 2× Protein-Substrate mix (final concentration 4 nM of EGFR and 50 ng/μl of Poly(Glu, Tyr)) was prepared in 1× Assay buffer and 4 μl of mixture per well was added into 384w white Reaction plate with NBS (Coming, Cat #4513). 4 μl of Poly(Glu, Tyr) substrate w/o EGFR in 1× buffer was used for negative control. Plates were centrifuged for 1 min at 200 g. Next step the Compounds were added to Reaction plate using Biomek station via following steps: 1 μl of 80× compounds (in DMSO) were mixed thoroughly with 39 μl of 2× 10 μM ATP in Assay Buffer, then 4 μl of this mixture was added to Reaction plate with 4 μl of Protein-Substrate mix. Plates were centrifuged for 1 min at 200 g and incubated for 1 h at rt. Next 4 μL of ADP-Glo reagent (Promega, ADP-Glo™ Kinase Assay, Cat #V9102) per well was added. Plates were incubated for 30 min at rt. Then 8 μL of Kinase detection reagent (Promega, ADP-Glo™ Kinase Assay, Cat #V9102) per well was added and the Luminescence was measured using Microplate Reader. The % inhibition was then used to calculate the EC₅₀ values. The results for certain compounds are presented in the Table D. The values of EC₅₀ shown as a letters A-E, where: A<0.001 μM; 0.001 μM<B<0.005 μM; 0.005 μM<C<

TABLE D
Potency of EGFR enzymatic activity Inhibition (WT)

| | EC₅₀, |
|---|---|
| Compound # | μM* |
| 99 | B |
| 100 | A |
| 104 | B |
| 114 | B |
| 115 | B |

*EC₅₀: Half maximal effective concentration: the concentration of a compound which induces a response halfway between the baseline and maximum after a specific exposure time;
EC₅₀: A ≤ 0.001 μM;
0.001 μM < B ≤ 0.005 μM;
0.005 μM < C ≤ 0.01 μM;
0.01 < D ≤ 0.05 μM;
E > 0.05

### Example E. Cellular Growth Inhibition Assay

Ba/F3(Empty vector) (Signosis), Ba/F3(Wild type) (Signosis), Ba/F3(L⁸⁵⁸R+T790M) (Signosis), Ba/F3(A767_dupASV) (Signosis), Ba/F3(D770_N771insSVD) (Signosis), Ba/F3(H773_V774insNPH) (Signosis) were seeded at a density of 4000 cells per well in a CellBIND® 384-well Flat Clear Bottom Black Polystyrene Microplates (Coming, USA, Cat #3770) in 45 μl total volume of RPMI (PanEco, Cat #C330, Russia) with 1000 FBS (HyClone, Cat #SV30160.03) and with 10 ng/ml rmIL-3 (RD systems, USA, Cat #403-ML-025) (for BaBF3(Empty vector)) and with 10 ng/ml rhEGF (ThermoFisher, USA, Cat #PHG0311L) (for BaCF3(Wild type)). 500× compounds solutions in DMSO (Sigma Cat #D2650) were prepared into Cmpnds plate (DiamondWell Plate, Axigen, Cat #P-384-120SQ-C—S) and DMSO only control was included. 1.l of 500× compounds (Cmpnds plate) was added to 49 l of culture medium into Dilution plate (Diamond Well Plate, Axigen, Cat #P-384-120SQ-C—S), mixed and then 5 μl of 10× compounds solutions were transferred to cells followed by centrifugation at 100 g for 1 min. Final DMSO concentration was 0.2% o. After 3 days of incubation 10.l of CellTiter-Glo (Promega, CAT #G7572) were added to the cells, plate was centrifuged at 100 g for 1 min and luminescence signal was measured using Microplate Reader (CLARIOStar).

TABLE E
Cellular Growth Inhibition Assay*

| | | Ba/F3 | Ba/F3 | Ba/F3 | Ba/F3 | Ba/F3 |
|---|---|---|---|---|---|---|
| | Ba/F3 | (L858R + | (A767_dup | (D770_N771 | (H773_V774 | (Empty |
| | (EGFRwt) | T790M) | ASV) | insSVD) | insSVD) | vector) |
| # | CC₅₀, μM | CC₅₀, μM | CC₅₀, μM | CC₅₀, μM | CC₅₀, μM | CC₅₀, μM |
| 12 | — | G | E | F | G | G |
| 29 | E | F | E | E | F | G |
| 99 | D | E | C | D | E | G |
| 100 | D | E | C | D | D | G |
| 101 | — | F | D | E | E | G |
| 104 | E | E | B | D | E | G |
| 114 | E | E | B | D | E | G |
| 115 | E | E | B | D | D | G |
| 117 | E | F | D | E | F | G |
| 118 | F | F | E | F | F | F |
| 119 | F | G | E | F | G | G |
| 123 | — | E | C | D | E | F |
| 127 | F | G | F | G | G | G |
| 128 | F | G | F | G | G | G |
| 129 | E | F | C | D | E | G |
| 131 | D | E | C | D | E | F |
| 132 | G | G | F | G | G | G |
| 133 | — | G | F | G | G | G |
| 135 | — | G | G | G | G | G |
| 136 | — | G | F | G | G | G |
| 137 | — | F | E | F | F | G |
| 139 | — | G | E | F | F | G |
| 140 | — | G | F | G | G | G |
| 141 | — | G | E | F | G | G |
| 143 | — | G | G | G | G | G |
| 145 | G | G | D | D | E | G |
| 147 | G | G | G | G | G | G |
| 148 | G | G | G | G | G | G |
| 149 | G | G | F | F | G | G |
| 150 | G | G | G | G | G | G |
| 153 | G | G | G | G | G | G |
| 157 | G | G | G | G | G | G |
| 158 | — | G | E | F | F | F |
| 159 | — | G | E | F | F | G |
| 160 | — | G | E | F | F | G |
| 161 | — | F | D | E | F | G |
| 162 | — | G | G | G | G | G |
| 163 | — | G | D | E | E | G |
| 164 | — | F | D | D | E | G |
| 166 | — | F | C | D | E | G |
| 167 | — | F | D | E | E | G |

*CC₅₀: Cytotoxic Concentration: the extract concentration that reduced the cell viability by 50% when compared to untreated controls;
CC₅₀: A ≤ 0.0001 μM;
0.0001 μM < B ≤ 0.001 μM;
0.001 μM < C ≤ 0.01 μM;
0.01 < D ≤ 0.1 μM;
0.1 < E ≤ 1 μM;
1 < F ≤ 10 μM;
G > 10

### Equivalents

Those skilled in the art will recognize, or be able to ascertain, using no more than routine experimentation, numerous equivalents to the specific embodiments described specifically herein. Such equivalents are intended to be encompassed in the scope of the following claims.

What is claimed is:

1. A compound of Formula (A): 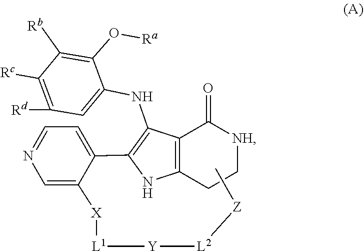
or a pharmaceutically acceptable salt, stereoisomer, solvate, or tautomer thereof, wherein
R^{a} is selected from C₁-C₆ alkyl, H, C₂-C₆ alkenyl, C₂-C₆ alkynyl, C₃-C₁₀ cycloalkyl, heterocyclyl, aryl, and heteroaryl, wherein the alkyl, alkenyl, alkynyl, cycloalkyl, heterocyclyl, aryl, or heteroaryl is optionally substituted with one or more substituents independently selected from halogen, OH, ═O, NR³R⁴, CN, NO₂, COOR¹, CONR³R⁴;
R^{b}, R^{c}, R^{d} are each independently selected from H, halogen, OH, NH₂, CN, NO₂, C₁-C₆ alkyl, and C₁-C₆ alkoxy, wherein alkyl or alkoxy is optionally substituted with one or more halogen, CN;
X is selected from 0, a bond, NR¹, C(R²)₂, S, S(O), S(O)₂, C(O), C(O)O, C(O)NR¹, OC(O)O, N(R²)C(O)NR², arenediyl;
R¹ is selected from H, C₁-C₆ alkyl, C₃-C₁₀ cycloalkyl, C₂-C₆ alkenyl, C₂-C₆ alkynyl, C₁-C₆ acyl, heterocycle, aryl, heteroaryl, and S(O)₂R² wherein the alkyl, alkenyl, alkynyl, heterocycle, cycloalkyl, aryl, or heteroaryl is optionally substituted with one or more substituents independently selected from halogen, OH, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, heteroaryl;
each R² is independently selected from H, C₁-C₆ alkyl, C₃-C₁₀ cycloalkyl, C₂-C₆ alkenyl, C₂-C₆ alkynyl, heterocycle, aryl, and heteroaryl wherein the alkyl, alkenyl, alkynyl, heterocycle, cycloalkyl, aryl, or heteroaryl is optionally substituted with one or more substituents independently selected from halogen, OH, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, heteroaryl;
Y is selected from a bond, O, NR¹, C(R²)₂, S, S(O), S(O)₂, C(O), C(O)O, C(O)NR¹, OC(O)O, N(R²)C(O)NR²;
Z is selected from a bond, O, C(R²)₂;
L¹ is selected from heterocyclediyl-C₁-C₁₀-alkanediyl, C₁-C₁₀ alkanediyl, C₂-C₁₀ alkenediyl, C₂-C₁₀ alkynediyl, C₃-C₁₀-cycloalkanediyl, C₃-C₁₀-cycloalkanediyl-C₁-C₁₀-alkanediyl, arenediyl, arenediyl-C₁-C₁₀-alkanediyl, heterocyclediyl, heterocyclediyl-C(O), heteroarenediyl, heteroarenediyl-C₁-C₁₀ alkanediyl, C₁-C₆ alkanediyl-O—C₁-C₆ alkanediyl, C₁-C₆ alkanediyl-NR¹—C₁-C₆ alkanediyl, C₁-C₆ alkanediyl-C(O)NR¹—C₁-C₆ alkanediyl, wherein alkanediyl, alkendiyl, alkynediyl, arenediyl, heterocyclediyl or heteroarenediyl heteroaryl is optionally substituted with one or more substituents independently selected from halogen, OH, oxo, CN, C₁-C₆ alkyl, C₁-C₆ alkoxy, C₁-C₆ alkyl-C₁-C₆ alkoxy, C₁-C₆ alkyl-NR¹—C₁-C₆ alkyl, NR³R⁴, —C(O)NR³R⁴, —S(O)C₁-C₆ alkyl, —S(O)₂NR³R⁴, aryl, and —S(O)₂C₁-C₆ alkyl;
L² is selected from C₁-C₁₀ alkanediyl, C₂-C₁₀ alkenediyl, C₂-C₁₀ alkynediyl, C₃-C₁₀-cycloalkanediyl, C₃-C₁₀-cycloalkanediyl-C₁-C₁₀-alkanediyl, arenediyl, arenediyl-C₁-C₁₀-alkanediyl, heterocyclediyl, heterocyclediyl-C₁-C₁₀-alkanediyl, heterocyclediyl-C(O), heteroarenediyl, heteroarenediyl-C₁-C₁₀ alkanediyl, C₁-C₆ alkanediyl-O—C₁-C₆ alkanediyl, C₁-C₆ alkanediyl-NR¹—C₁-C₆ alkanediyl, C₁-C₆ alkanediyl-C(O)NR¹—C₁-C₆ alkanediyl, wherein alkanediyl, alkendiyl, alkynediyl, arenediyl, heterocyclediyl or heteroarenediyl heteroaryl is optionally substituted with one or more substituents independently selected from halogen, OH, oxo, CN, C₁-C₆ alkyl, C₁-C₆ alkoxy, C₁-C₆ alkyl-C₁-C₆ alkoxy, C₁-C₆ alkyl-NR¹—C₁-C₆ alkyl, NR³R⁴, —C(O)NR³R⁴, —S(O)C₁-C₆ alkyl, —S(O)₂NR³R⁴, aryl, and —S(O)₂C₁-C₆ alkyl;
R³ and R⁴ are each independently selected from H, C₁-C₆ alkyl, C₁-C₆ acyl and C₃-C₁₀ cycloalkyl;
or R³ and R⁴ together with the atoms to which they are attached and any intervening atoms, form a 5-14 membered heterocycle, wherein the heterocycle is optionally substituted with one or more substituents independently selected from halogen, OH, ═O, CN, NO₂, C₁-C₆ alkyl, C₁-C₆ alkoxy;
wherein,
aryl is cyclic, aromatic hydrocarbon groups that have 1 to 3 aromatic rings;
arenediyl is an organic radical derived from an aromatic divalent radical by removal of two hydrogens;
heterocyclyl is saturated or partially unsaturated 3-10 membered monocyclic, 7-12 membered bicyclic (fused, bridged, or spiro rings), or 11-14 membered tricyclic ring system (fused, bridged, or spiro rings) having one or more heteroatoms selected from O, N, S, P, Se, or B;
heterocyclediyl is a divalent functional group derived from heterocycle by the removal of two hydrogen atoms from ring atoms;
heteroaryl is a monovalent monocyclic or a polycyclic aromatic radical of 5 to 24 ring atoms, containing one or more ring heteroatoms selected from N, O, S, P, or B, the remaining ring atoms being C;
heteroarenediyl is a divalent functional group derived from heteroarenes by the removal of two hydrogen atoms from ring atoms.

2. The compound of claim 1, wherein the compound is of Formula (I): 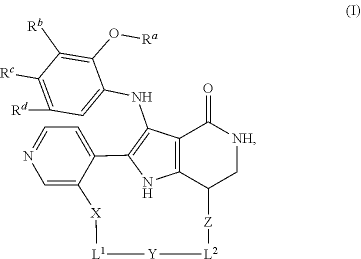
or a pharmaceutically acceptable salt, stereoisomer, solvate, or tautomer thereof.

3. The compound of claim 2, wherein the compound is of Formula (I′) or Formula (I″): 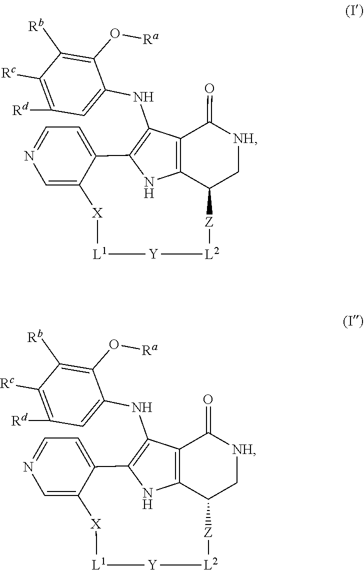
or a pharmaceutically acceptable salt, stereoisomer, solvate, or tautomer thereof.

4. The compound of claim 1, wherein the compound is of Formula (II): 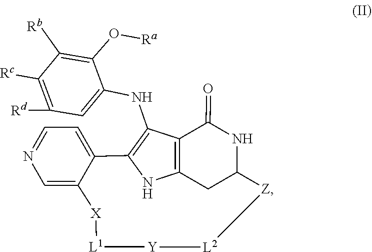
or a pharmaceutically acceptable salt, stereoisomer, solvate, or tautomer thereof.

5. The compound of claim 4, wherein the compound is of Formula (II′) or Formula (II″): 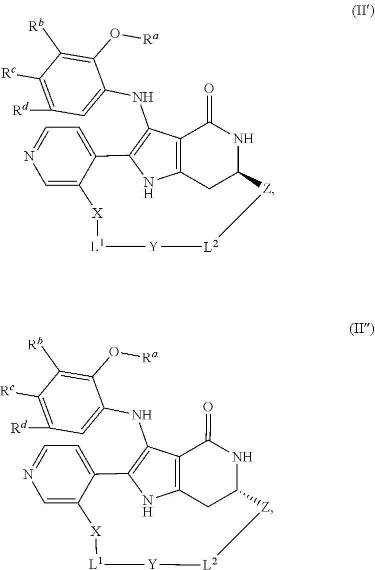
or a pharmaceutically acceptable salt, stereoisomer, solvate, or tautomer thereof.

6. The compound of anyone of claims 1-5, or a pharmaceutically acceptable salt, stereoisomer, solvate, or tautomer thereof, wherein
R^{a} is selected from H, C₁-C₆ alkyl, C₂-C₆ alkenyl, wherein the alkyl or alkenyl is optionally substituted with one or more substituents independently selected from halogen, OH, ═O, NH₂, CN, NO₂;
R^{b}, R^{c}, R^{d} are each independently selected from H, halogen, OH, NH₂, CN, NO₂, C₁-C₆ alkyl, C₁-C₆ alkoxy;
X is selected from a bond, —O—, —NH—, —N(CH₃)—, —CH₂—, —S—, —S(O)—, —S(O)₂—, —C(O)—, —C(O)O—, —C(O)NH—, —OC(O)O—, —NHC(O)NH—;
Y is selected from a bond, —O—, —NH—, —N(CH₃)—, —N(CH(CH₃)₂)—, 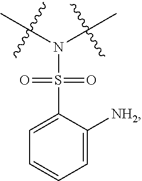 [SMILES: *N(*)S(=O)(=O)c1ccccc1N]
—CH₂—, —S—, —S(O)—, —S(O)₂—, —C(O)—, —C(O)O—, —C(O)NH—, —OC(O)O—, —NHC(O)NH—;
Z is selected from a bond, —O—, —CH₂—;
L¹ is selected from —CH₂—, —(CH₂)₂—, —(CH₂)₃—, —(CH₂)₄—, —(CH₂)₅—, —(CH₂)₆—, —CH₂CH(CH₃)CH₂—, —CH(CH₃)(CH₂)₂—, —CH₂CH(CH₃)(CH₂)₂—, —(CH₂)₂CH(CH₃)(CH₂)—, —CH₂CH(CH₃)CH(CH₃)(CH₂)₂—, —(CH₂)₂CH(CH₃)(CH₂)₃—, —(CH₂)₂CH(CH₃)CH(CH₃)(CH₂)₂—, —CH₂—O—CH₂—, —CH₂—O—(CH₂)₂—, —(CH₂)₂—O—(CH₂)₂—, —CH═CH—, —CH₂—CH═CH—, —CH₂—C(CH₃)═CH—, —CH₂—CH═C(CH₃)—, —CH₂—CH═CH—CH₂—, —CH₂—C(CH₃)═CH—CH₂—, —(CH₂)₂C(CH₃)═CH—, —(CH₂)₂CH═CHCH₂—, —(CH₂)₂C(CH₃)═CHCH₂—, —(CH₂)₂CH═C(CH₃)CH₂—, —(CH₂)₂C(CH₃)═C(CH₃)CH₂—, —(CH₂)₂C(CH₃)═CH(CH₂)₂—, —(CH₂)₂C(CH₃)═C(CH₃)(CH₂)₂—, —C≡C—, —C≡CCH₂—, —C—CCH(CH₃)—, —CH₂CH₂CH₂—NH—CH₂CH₂—, —CH₂CH₂CH₂—N(CH₃)— 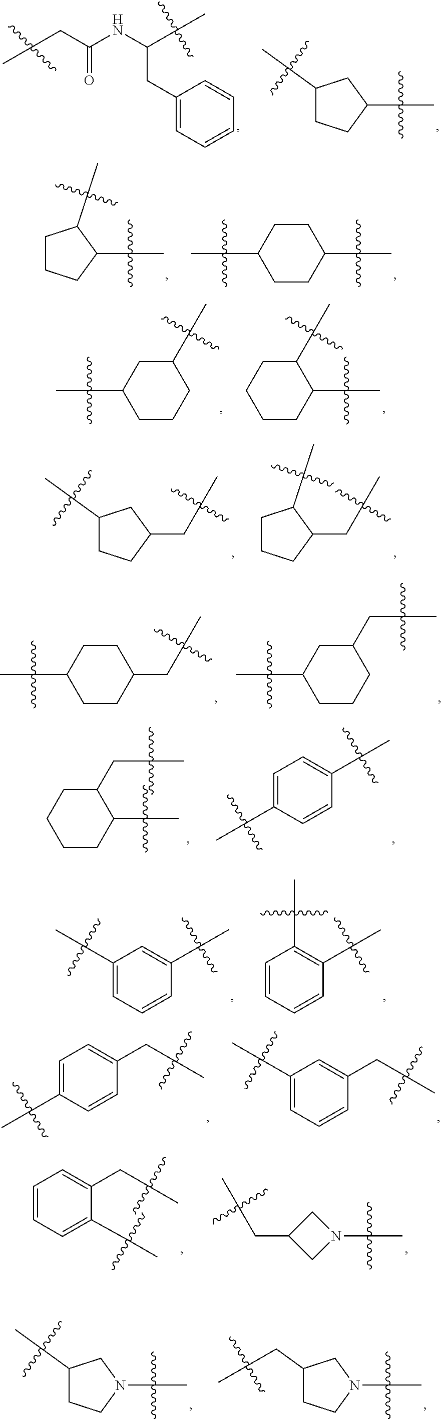 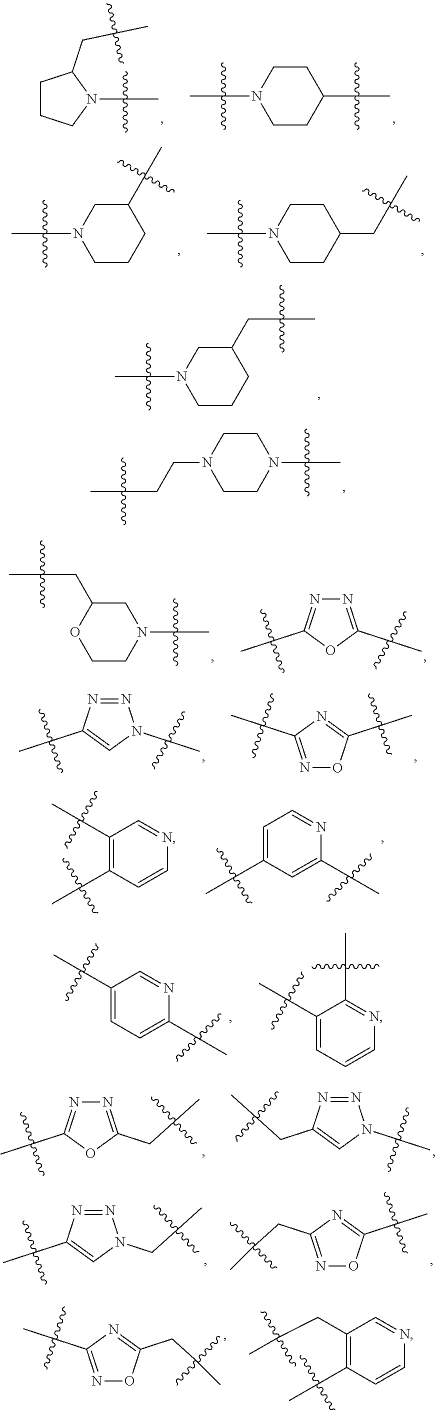 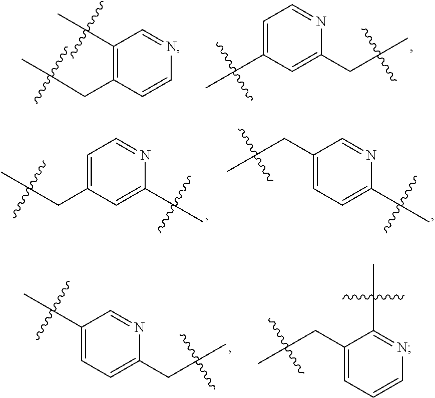 [SMILES: *Cc1ccncc1* ; *Cc1cc(*)ccn1 ; *Cc1ccnc(*)c1 ; *Cc1ccc(*)nc1 ; *Cc1ccc(*)cn1 ; *Cc1cccnc1*]
L² is selected from —CH₂—, —(CH₂)₂—, —(CH₂)₃—, —(CH₂)₄—, —(CH₂)₅—, —(CH₂)₆—, —CH₂CH(CH₃)CH₂—, —CH(CH₃)(CH₂)₂—, —CH₂CH(CH₃)(CH₂)₂—, —(CH₂)₂CH(CH₃)(CH₂)—, —CH₂CH(CH₃)CH(CH₃)(CH₂)₂—, —(CH₂)₂CH(CH₃)(CH₂)₃—, —(CH₂)₂CH(CH₃)CH(CH₃)(CH₂)₂—, —CH₂—O—CH₂—, —CH₂—O—(CH₂)₂—, —(CH₂)₂—O—(CH₂)₂—, —CH═CH—, —CH₂—CH═CH—, —CH₂—C(CH₃)═CH—, —CH₂—CH═C(CH₃)—, —CH₂—CH═CH—CH₂—, —CH₂—C(CH₃)═CH—CH₂—, —(CH₂)₂C(CH₃)═CH—, —(CH₂)₂CH═CHCH₂—, —(CH₂)₂C(CH₃)═CHCH₂—, —(CH₂)₂CH═C(CH₃)CH₂—, —(CH₂)₂C(CH₃)═C(CH₃)CH₂—, —(CH₂)₂C(CH₃)═CH(CH₂)₂—, —(CH₂)₂C(CH₃)═C(CH₃)(CH₂)₂—, —C—C—, —C—CCH₂—, —C—C—CH(CH₃)—, —CH₂—C(O)NH—CH₂—, —CH₂—C(O)N(CH₃)—CH₂—, —CH₂—H 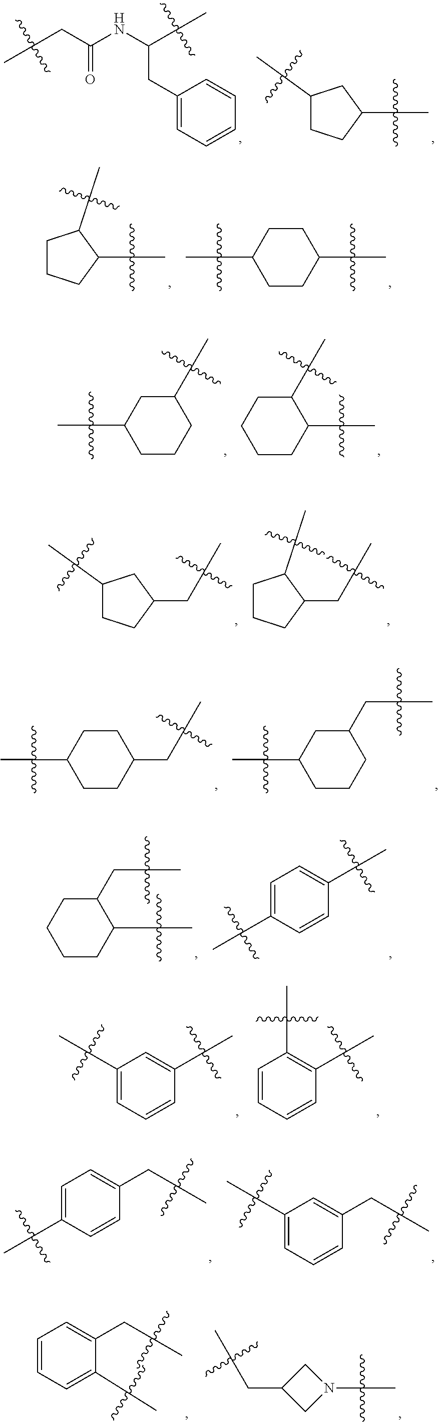 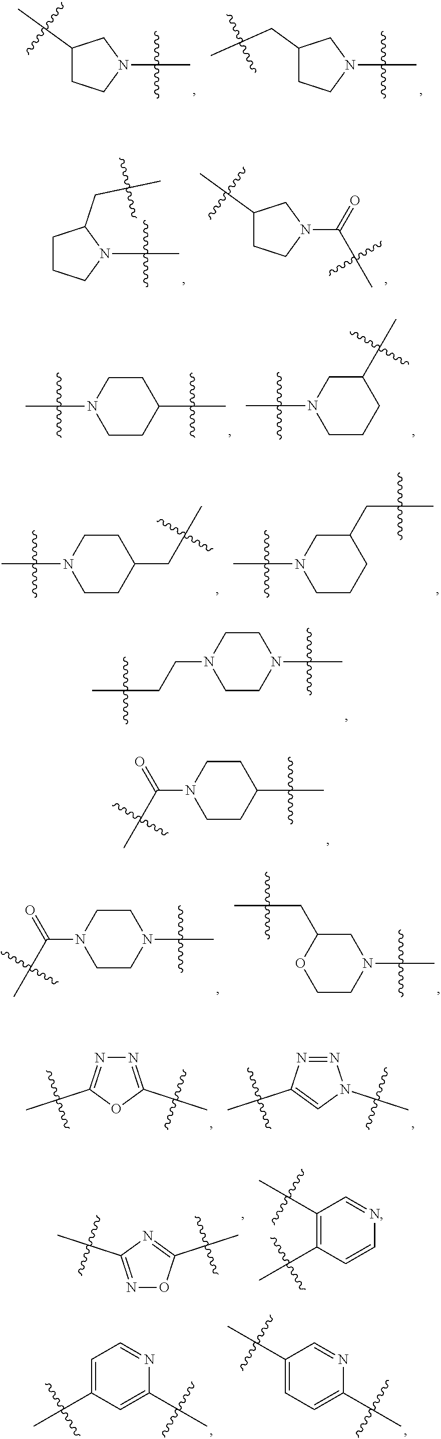 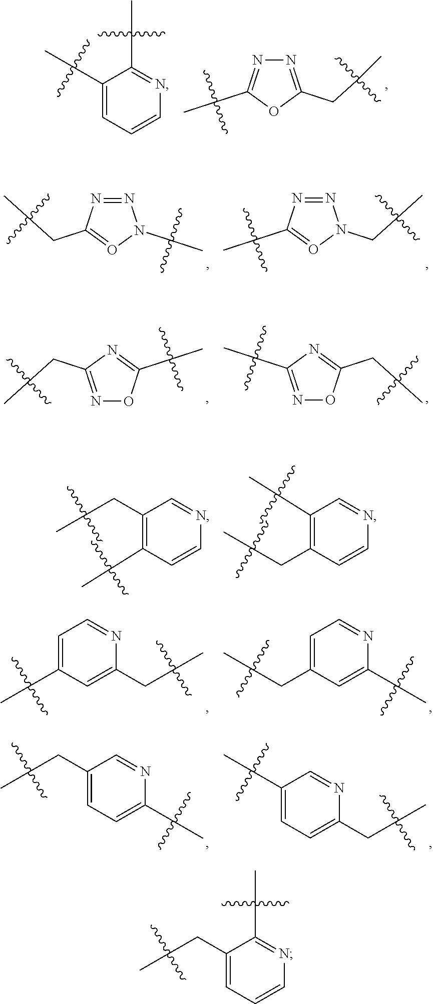
wherein L¹ and L² are optionally substituted with one or more substituents independently selected from halogen, OH, oxo, CN, NH₂.

7. The compound of claim 1, wherein the compound is of Formula (A-I): 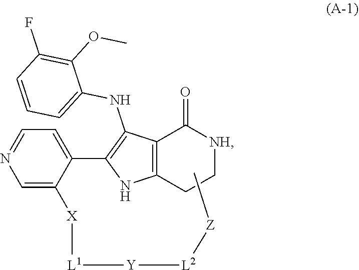
or a pharmaceutically acceptable salt, stereoisomer, solvate, or tautomer thereof.

8. The compound of claim 7, wherein the compound is of Formula (A-1-a): 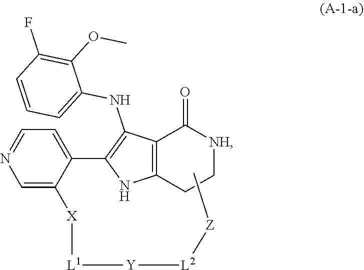
or a pharmaceutically acceptable salt, stereoisomer, solvate, or tautomer thereof.

9. The compound of claim 7, wherein the compound is of Formula (A-1-b): 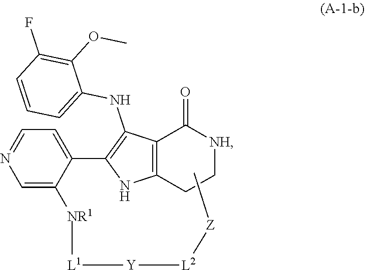
or a pharmaceutically acceptable salt, stereoisomer, solvate, or tautomer thereof.

10. The compound of claim 2, wherein the compound is of Formula (I-A): 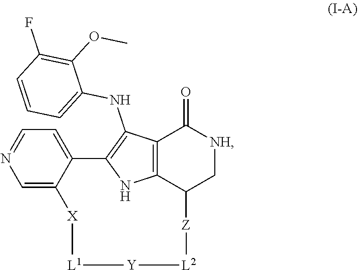
or a pharmaceutically acceptable salt, stereoisomer, solvate, or tautomer thereof.

11. The compound of claim 10, wherein the compound is of Formula (I-A-I): 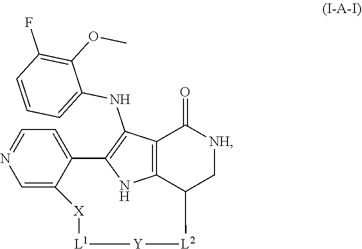
or a pharmaceutically acceptable salt, stereoisomer, solvate, or tautomer thereof.

12. The compound of claim 11, wherein the compound is of Formula: 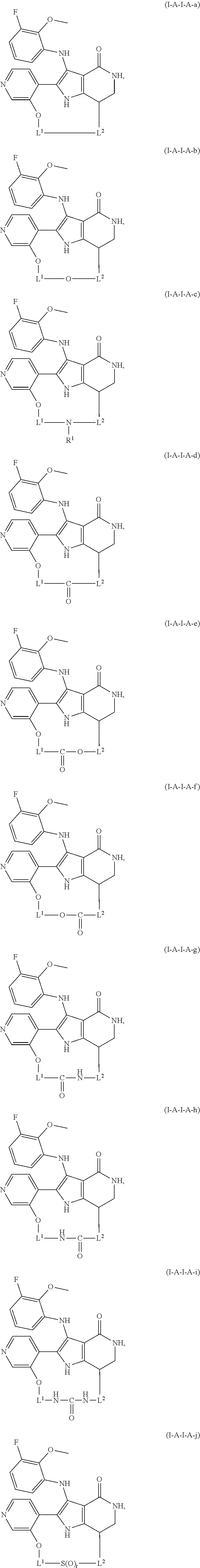 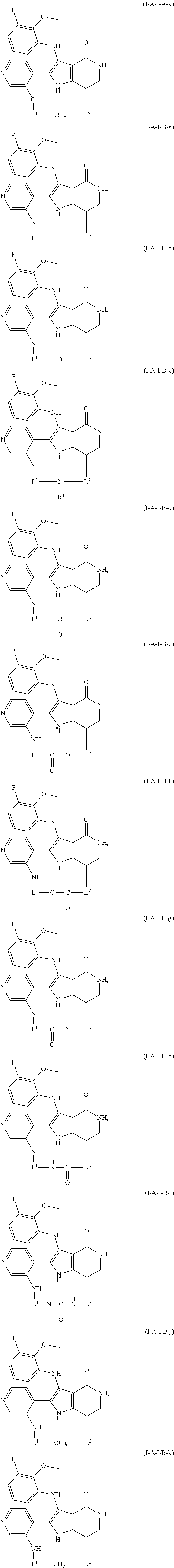
or a pharmaceutically acceptable salt, stereoisomer, solvate, or tautomer thereof, wherein t is an integer selected from 0, 1, and 2 and all other variables are as defined herein.

13. The compound of claim 4, wherein the compound is of Formula (II-A): 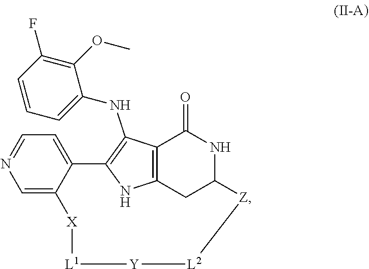
or a pharmaceutically acceptable salt, stereoisomer, solvate, or tautomer thereof.

14. The compound of claim 13, wherein the compound is of Formula (II-A-I): 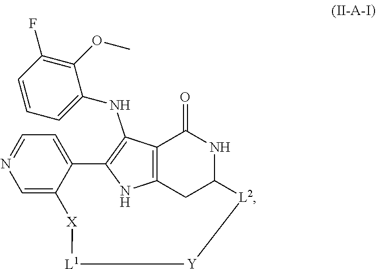
or a pharmaceutically acceptable salt, stereoisomer, solvate, or tautomer thereof.

15. The compound of claim 14, wherein the compound is of Formula: 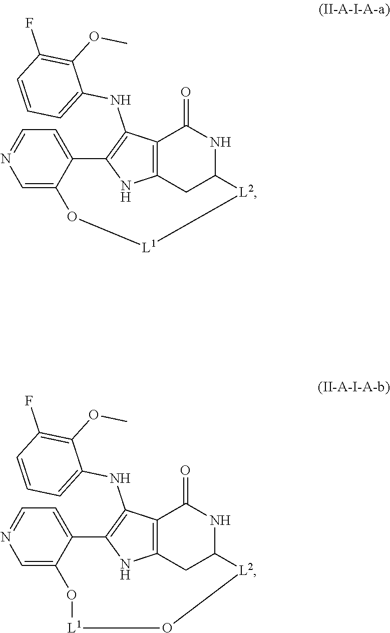 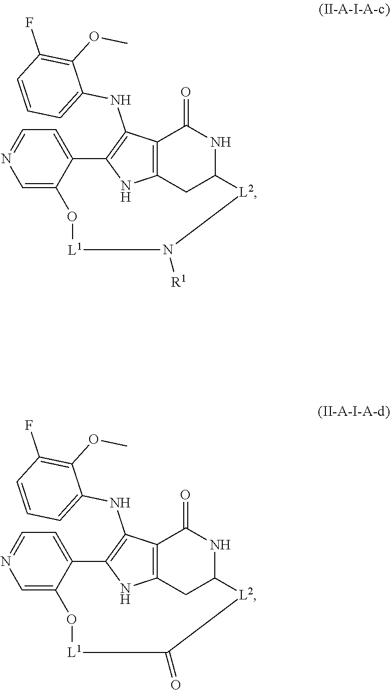 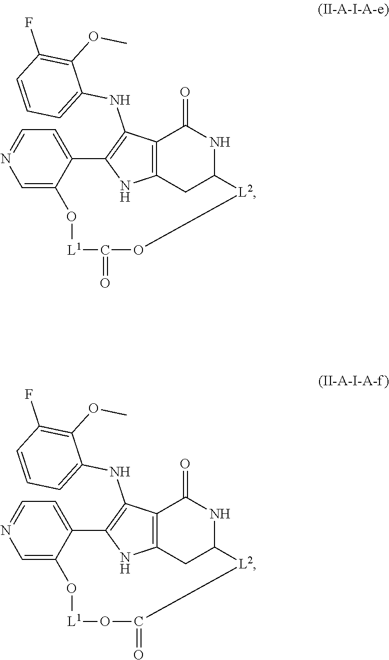 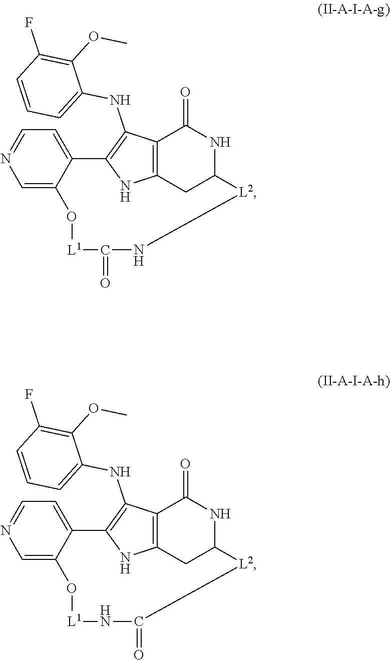 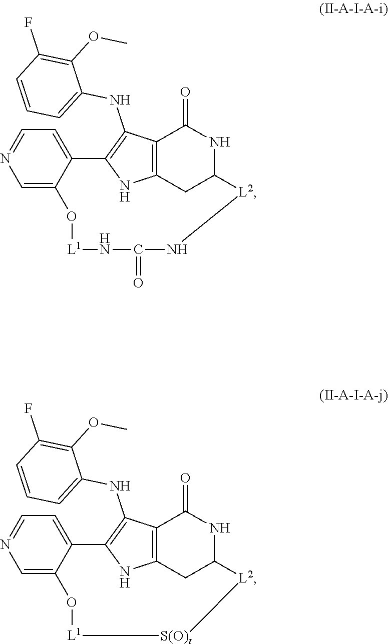 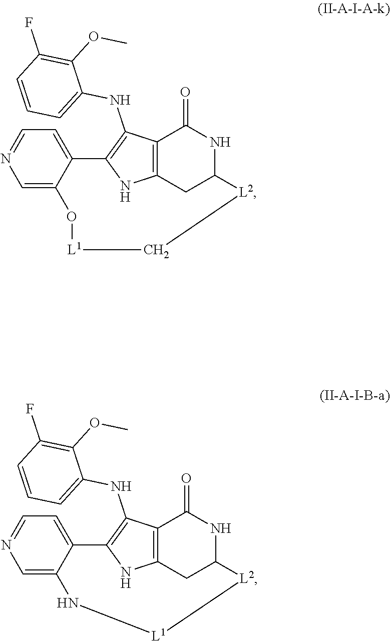 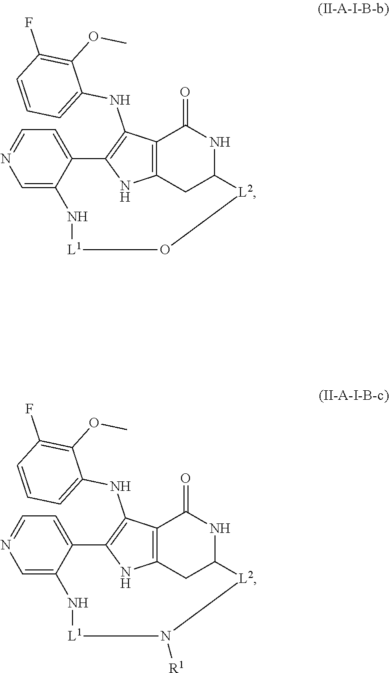 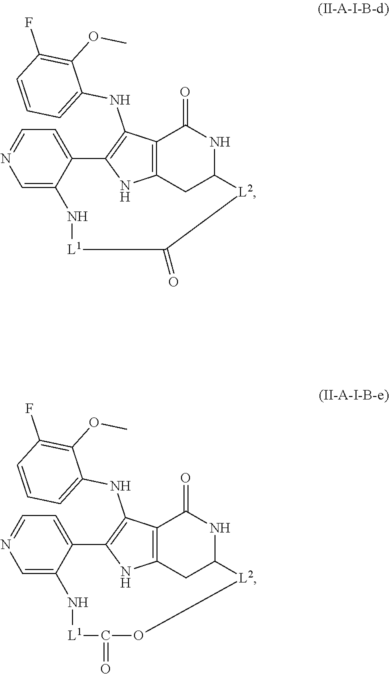 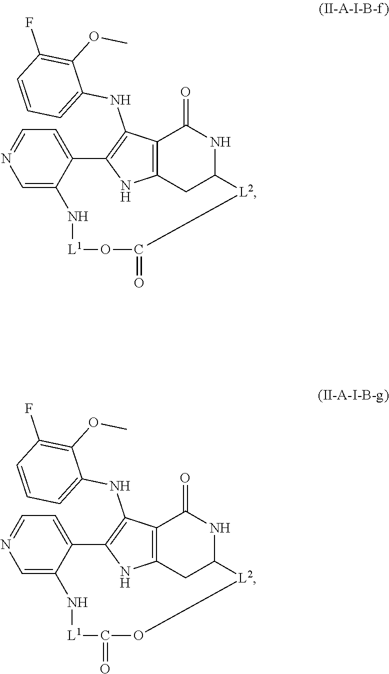 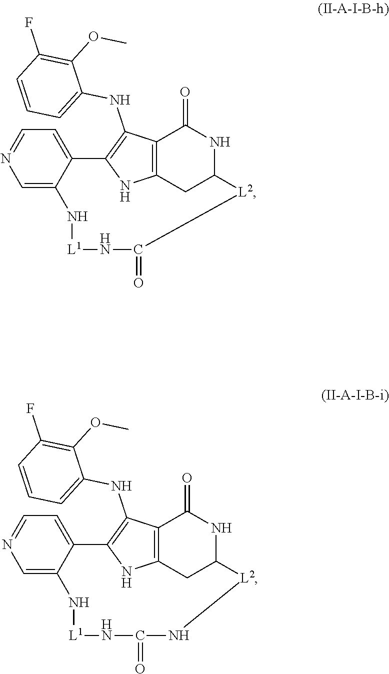 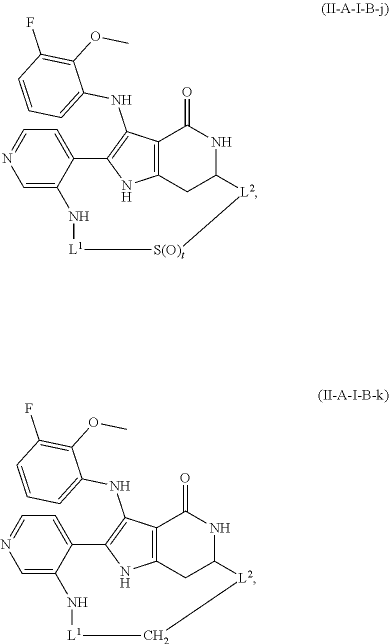
or a pharmaceutically acceptable salt, stereoisomer, solvate, or tautomer thereof, wherein t is an integer selected from 0, 1, and 2 and all other variables are as defined herein.

16. The compound of claim 1, wherein the compound is of Formula (A-A): 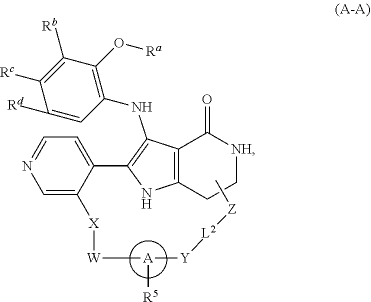
or a pharmaceutically acceptable salt, stereoisomer, solvate, or tautomer thereof, wherein
R^{a} is selected from C₁-C₆ alkyl, H, C₂-C₆ alkenyl, C₂-C₆ alkynyl, C₃-C₁₀ cycloalkyl, heterocyclyl, aryl, and heteroaryl, wherein the alkyl, alkenyl, alkynyl, cycloalkyl, heterocyclyl, aryl, or heteroaryl is optionally substituted with one or more substituents independently selected from halogen, OH, ═O, NR³R⁴, CN, NO₂, COOR¹, CONR³R⁴;
R^{b}, R^{c}, R^{d} are each independently selected from H, halogen, OH, NH₂, CN, NO₂, C₁-C₆ alkyl, and C₁-C₆ alkoxy, wherein alkyl or alkoxy is optionally substituted with one or more halogen, CN;
X is selected from a bond, O, NR¹, C(R²)₂, S, S(O), S(O)₂, C(O), C(O)O, C(O)NR¹, OC(O)O, N(R²)C(O)NR², arenediyl;
R¹ is selected from H, C₁-C₆ alkyl, C₃-C₁₀ cycloalkyl, C₂-C₆ alkenyl, C₂-C₆ alkynyl, C₁-C₆ acyl, heterocycle, aryl, heteroaryl, and S(O)₂R² wherein the alkyl, alkenyl, alkynyl, heterocycle, cycloalkyl, aryl, or heteroaryl is optionally substituted with one or more substituents independently selected from halogen, OH, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, heteroaryl;
each R² is independently selected from H, C₁-C₆ alkyl, C₃-C₁₀ cycloalkyl, C₂-C₆ alkenyl, C₂-C₆ alkynyl, heterocycle, aryl, and heteroaryl wherein the alkyl, alkenyl, alkynyl, heterocycle, cycloalkyl, aryl, or heteroaryl is optionally substituted with one or more substituents independently selected from halogen, OH, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, heteroaryl;
Y is selected from a bond, O, NR¹, C(R²)₂, S, S(O), S(O)₂, C(O), C(O)O, C(O)NR¹, OC(O)O, N(R²)C(O)NR²;
Z is selected from a bond, O, C(R²)₂;
Ring A is selected from C₃-C₁₀-cycloalkane, arene, heterocycle, heteroarene;
W is (CH₂)_{w};
w is an integer selected from 0, 1, 2, 3, 4, 5, 6, 7, 8, 9, and 10;
L² is selected from C₁-C₁₀ alkanediyl, C₂-C₁₀ alkenediyl, C₂-C₁₀ alkynediyl, C₃-C₁₀-cycloalkanediyl, C₃-C₁₀-cycloalkanediyl-C₁-C₁₀-alkanediyl, arenediyl, arenediyl-C₁-C₁₀-alkanediyl, heterocyclediyl, heterocyclediyl-C₁-C₁₀-alkanediyl, heterocyclediyl-C(O), heteroarenediyl, heteroarenediyl-C₁-C₁₀ alkanediyl, C₁-C₆ alkanediyl-O—C₁-C₆ alkanediyl, C₁-C₆ alkanediyl-NR¹—C₁-C₆ alkanediyl, C₁-C₆ alkanediyl-C(O)NR¹—C₁-C₆ alkanediyl, wherein alkanediyl, alkendiyl, alkynediyl, arenediyl, heterocyclediyl or heteroarenediyl heteroaryl is optionally substituted with one or more substituents independently selected from halogen, OH, oxo, CN, C₁-C₆ alkyl, C₁-C₆ alkoxy, C₁-C₆ alkyl-C₁-C₆ alkoxy, C₁-C₆ alkyl-NR¹—C₁-C₆ alkyl, NR³R⁴, —C(O)NR³R⁴, —S(O)C₁-C₆ alkyl, —S(O)₂NR³R⁴, aryl, and —S(O)₂C₁-C₆ alkyl;
R³ and R⁴ are each independently selected from H, C₁-C₆ alkyl, C₁-C₆ acyl and C₃-C₁₀ cycloalkyl;
or R³ and R⁴ together with the atoms to which they are attached and any intervening atoms, form a 5-14 membered heterocycle, wherein the heterocycle is optionally substituted with one or more substituents independently selected from halogen, OH, ═O, CN, NO₂, C₁-C₆ alkyl, C₁-C₆ alkoxy;
R⁵ is selected from H, halogen, OH, oxo, CN, C₁-C₆ alkyl, C₁-C₆ alkoxy, C₁-C₆ alkyl-C₁-C₆ alkoxy, C₁-C₆ alkyl-NR¹—C₁-C₆ alkyl, NR³R⁴, —C(O)NR³R⁴, —S(O)C₁-C₆ alkyl, —S(O)₂NR³R⁴, and —S(O)₂C₁-C₆ alkyl.

17. The compound of claim 1, wherein the compound is of Formula (A-B): 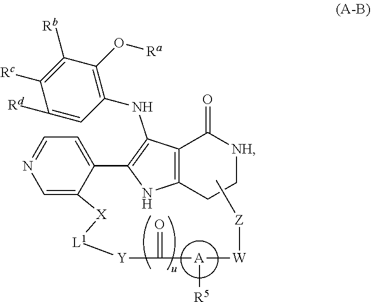
or a pharmaceutically acceptable salt, stereoisomer, solvate, or tautomer thereof, wherein
R^{a} is selected from C₁-C₆ alkyl, H, C₂-C₆ alkenyl, C₂-C₆ alkynyl, C₃-C₁₀ cycloalkyl, heterocyclyl, aryl, and heteroaryl, wherein the alkyl, alkenyl, alkynyl, cycloalkyl, heterocyclyl, aryl, or heteroaryl is optionally substituted with one or more substituents independently selected from halogen, OH, ═O, NR³R⁴, CN, NO₂, COOR¹, CONR³R⁴;
R^{b}, R^{c}, R^{d} are each independently selected from H, halogen, OH, NH₂, CN, NO₂, C₁-C₆ alkyl, and C₁-C₆ alkoxy, wherein alkyl or alkoxy is optionally substituted with one or more halogen, CN;
X is selected from a bond, O, NR¹, C(R²)₂, S, S(O), S(O)₂, C(O), C(O)O, C(O)NR¹, OC(O)O, N(R²)C(O)NR², arenediyl;
R¹ is selected from H, C₁-C₆ alkyl, C₃-C₁₀ cycloalkyl, C₂-C₆ alkenyl, C₂-C₆ alkynyl, C₁-C₆ acyl, heterocycle, aryl, heteroaryl, and S(O)₂R² wherein the alkyl, alkenyl, alkynyl, heterocycle, cycloalkyl, aryl, or heteroaryl is optionally substituted with one or more substituents independently selected from halogen, OH, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, heteroaryl;
each R² is independently selected from H, C₁-C₆ alkyl, C₃-C₁₀ cycloalkyl, C₂-C₆ alkenyl, C₂-C₆ alkynyl, heterocycle, aryl, and heteroaryl wherein the alkyl, alkenyl, alkynyl, heterocycle, cycloalkyl, aryl, or heteroaryl is optionally substituted with one or more substituents independently selected from halogen, OH, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, heteroaryl;
L¹ is selected from C₁-C₁₀ alkanediyl, C₂-C₁₀ alkenediyl, C₂-C₁₀ alkynediyl, C₃-C₁₀-cycloalkanediyl, C₃-C₁₀-cycloalkanediyl-C₁-C₁₀-alkanediyl, arenediyl, arenediyl-C₁-C₁₀-alkanediyl, heterocyclediyl, heterocyclediyl-C₁-C₁₀-alkanediyl, heterocyclediyl-C(O), heteroarenediyl, heteroarenediyl-C₁-C₁₀ alkanediyl, C₁-C₆ alkanediyl-O—C₁-C₆ alkanediyl, C₁-C₆ alkanediyl-NR¹—C₁-C₆ alkanediyl, C₁-C₆ alkanediyl-C(O)NR¹—C₁-C₆ alkanediyl, wherein alkanediyl, alkendiyl, alkynediyl, arenediyl, heterocyclediyl or heteroarenediyl heteroaryl is optionally substituted with one or more substituents independently selected from halogen, OH, oxo, CN, C₁-C₆ alkyl, C₁-C₆ alkoxy, C₁-C₆ alkyl-C₁-C₆ alkoxy, C₁-C₆ alkyl-NR¹—C₁-C₆ alkyl, NR³R⁴, —C(O)NR³R⁴, —S(O)C₁-C₆ alkyl, —S(O)₂NR³R⁴, aryl, and —S(O)₂C₁-C₆ alkyl;
Y is selected from a bond, O, NR¹, C(R²)₂, S, S(O), S(O)₂, C(O), C(O)O, C(O)NR¹, OC(O)O, N(R²)C(O)NR²;
Z is selected from a bond, O, C(R²)₂;
Ring A is selected from C₃-C₁₀-cycloalkane, arene, heterocycle, heteroarene;
W is (CH₂)_{w};
w is an integer selected from 0, 1, 2, 3, 4, 5, 6, 7, 8, 9, and 10;
u is an integer selected from 0 and 1;
R³ and R⁴ are each independently selected from H, C₁-C₆ alkyl, C₁-C₆ acyl and C₃-C₁₀ cycloalkyl;
or R³ and R⁴ together with the atoms to which they are attached and any intervening atoms, form a 5-14 membered heterocycle, wherein the heterocycle is optionally substituted with one or more substituents independently selected from halogen, OH, ═O, CN, NO₂, C₁-C₆ alkyl, C₁-C₆ alkoxy;
R⁵ is selected from H, halogen, OH, oxo, CN, C₁-C₆ alkyl, C₁-C₆ alkoxy, C₁-C₆ alkyl-C₁-C₆ alkoxy, C₁-C₆ alkyl-NR¹—C₁-C₆ alkyl, NR³R⁴, —C(O)NR³R⁴, —S(O)C₁-C₆ alkyl, —S(O)₂NR³R⁴, and —S(O)₂C₁-C₆ alkyl.

18. A compound selected from:

| # | Structure | IUPAC name |
|---|---|---|
| 1 | 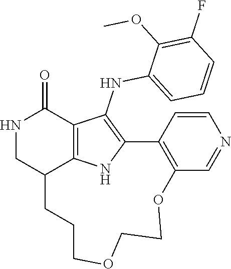 [SMILES: COc1c(F)cccc1Nc1c2[nH]c3c1C(=O)NCC3CCCOCCOc1cnccc1-2] | 22-[(3-fluoro-2-methoxyphenyl)amino]- 8,11-dioxa-5,17,21- triazatetracyclo[17.2.1.0^{2,7}.0^{15,20}]docosa- 1(22),2,4,6,19-pentaen-18-one |
| 2 | 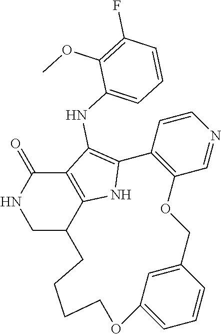 [SMILES: COc1c(F)cccc1Nc1c2[nH]c3c1C(=O)NCC3CCCCOc1cccc(c1)COc1cnccc1-2] | 28-[(3-fluoro-2-methoxyphenyl)amino]- 3,22-dioxa-6,11,15- triazapentacyclo[21.3.1.1^{10,13}.0^{4,9}.0^{12,17}]- octacosa-1(27),4,6,8,10(28),12,23,25- octaen-14-one |
| 3 | 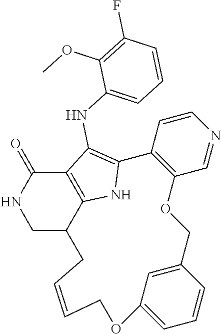 [SMILES: COc1c(F)cccc1Nc1c2[nH]c3c1C(=O)NCC3C/C=C\COc1cccc(c1)COc1cnccc1-2] | (19Z)-28-[(3-fluoro-2- methoxyphenyl)amino]-3,22-dioxa-6,11,15- triazapentacyclo[21.3.1.1^{10,13}.0^{4,9}.0^{12,17}]- octacosa-1(27),4,6,8,10(28),12,19,23,25- nonaen-14-one |
| 4 | 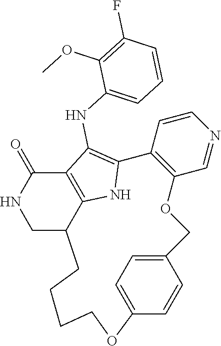 [SMILES: COc1c(F)cccc1Nc1c2[nH]c3c1C(=O)NCC3CCCCOc1ccc(cc1)COc1cnccc1-2] | 28-[(3-fluoro-2-methoxyphenyl)amino]- 3,22-dioxa-6,11,15- triazapentacyclo[21.2.2.1^{10,13}.0^{4,9}.0^{12,17}]- octacosa-1(25)-4,6,8,10(28),12,23,26- octaen-14-one |
| 5 | 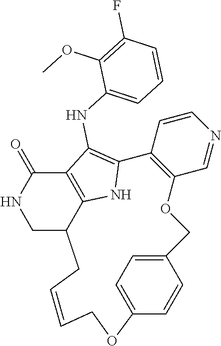 [SMILES: COc1c(F)cccc1Nc1c2[nH]c3c1C(=O)NCC3C/C=C\COc1ccc(cc1)COc1cnccc1-2] | (19Z)-28-[(3-fluoro-2- methoxyphenyl)amino]-3,22-dioxa-6,11,15- triazapentacyclo[21.2.2.1^{10,13}.0^{4,9}.0^{12,17}]- octacosa-1(25),4,6,8,10(28),12,19,23,26- nonaen-14-one |
| 6 | 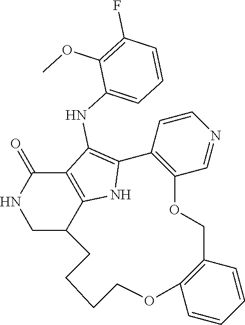 [SMILES: COc1c(F)cccc1Nc1c2[nH]c3c1C(=O)NCC3CCCCOc1ccccc1COc1cnccc1-2] | 28-[(3-fluoro-2-methoxyphenyl)amino]- 8,16-dioxa-5,23,27- triazapentacyclo[23.2.1.0^{2,7}.0^{10,15}.0^{21,26}]- octacosa-1(28),2,4,6,10,12,14,25-octaen-24- one |
| 7 | 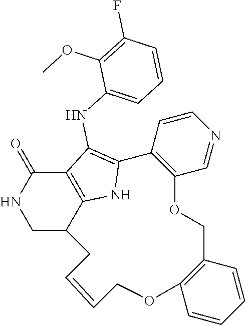 [SMILES: COc1c(F)cccc1Nc1c2[nH]c3c1C(=O)NCC3C/C=C\COc1ccccc1COc1cnccc1-2] | (18Z)-28-[(3-fluoro-2- methoxyphenyl)amino]-8,16-dioxa-5,23,27- triazapentacyclo[23.2.1.0^{2,7}.0^{10,15}.0^{21,26}]- octacosa-1(28),2,4,6,10,12,14,18,25- nonaen-24-one |
| 8 | 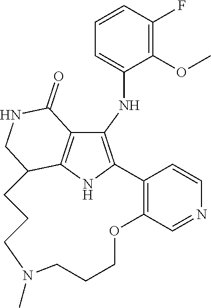 [SMILES: COc1c(F)cccc1Nc1c2[nH]c3c1C(=O)NCC3CCCN(C)CCCOc1cnccc1-2] | 23-[(3-fluoro-2-methoxyphenyl)amino]-12- methyl-8-oxa-5,12,18,22- tetraazatetracyclo[18.2.1.0^{2,7}.0^{2,7}.0^{16,21}]tricosa- 1(23),2,4,6,20-pentaen-19-one |
| 9 | 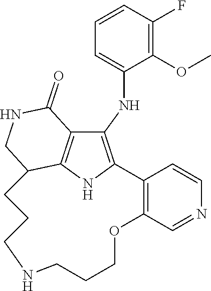 [SMILES: COc1c(F)cccc1Nc1c2[nH]c3c1C(=O)NCC3CCCNCCCOc1cnccc1-2] | 23-[(3-fluoro-2-methoxyphenyl)amino]-8- oxa-5,12,18,22- tetraazatetracyclo[18.2.1.0^{2,7}.0^{16,21}]tricosa- 1(23),2,4,6,20-pentaen-19-one |
| 10 | 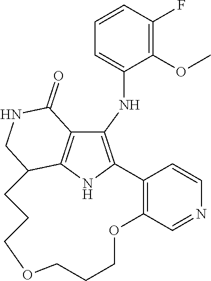 [SMILES: COc1c(F)cccc1Nc1c2[nH]c3c1C(=O)NCC3CCCOCCCOc1cnccc1-2] | 23-[(3-fluoro-2-methoxyphenyl)amino]- 8,12-dioxa-5,18,22- triazatetracyclo[18.2.1.0^{2,7}.0^{16,21}]tricosa- 1(23),2,4,6,20-pentaen-19-one |
| 11 | 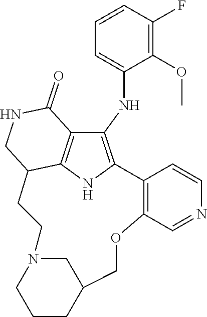 [SMILES: COc1c(F)cccc1Nc1c2[nH]c3c1C(=O)NCC3CCN1CCCC(COc3cnccc3-2)C1] | 25-[(3-fluoro-2-methoxyphenyl)amino]-18-oxa- 1,6,10,15- tetraazapentacyclo[18.3.1.1^{8,11}.0^{4,9}.0^{12,17}]- pentacosa-8,11(25),12,14,16-pentaen-7-one |
| 12 | 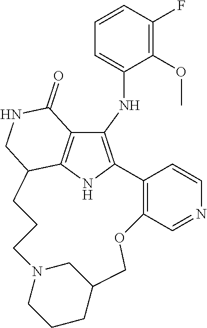 [SMILES: COc1c(F)cccc1Nc1c2[nH]c3c1C(=O)NCC3CCCN1CCCC(COc3cnccc3-2)C1] | 26-[(3-fluoro-2-methoxyphenyl)amino]-3-oxa- 6,11,15,21- tetraazapentacyclo[19.3.1.1^{10,13}.0^{4,9}.0^{12,17}]- hexacosa-4,6,8,10(26),12-pentaen-14-one |
| 13 | 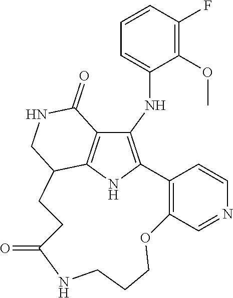 [SMILES: COc1c(F)cccc1Nc1c2[nH]c3c1C(=O)NCC3CCC(=O)NCCCOc1cnccc1-2] | 23-[(3-fluoro-2-methoxyphenyl)amino]-8- oxa-5,12,18,22- tetraazatetracyclo[18.2.1.0^{2,7}.0^{16,21}]tricosa- 1(23),2,4,6,20-pentaene-13,19-dione |
| 14 | 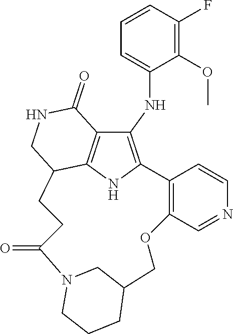 [SMILES: COc1c(F)cccc1Nc1c2[nH]c3c1C(=O)NCC3CCC(=O)N1CCCC(COc3cnccc3-2)C1] | 26-[(3-fluoro-2-methoxyphenyl)amino]-3-oxa- 6,11,15,21- tetraazapentacyclo[19.3.1.1^{10,13}.0^{4,9}.0^{12,17}]- hexacosa-4,6,8,10(26),12-pentaene-14,20-dione |
| 15 | 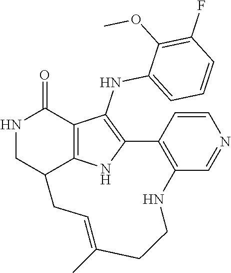 [SMILES: COc1c(F)cccc1Nc1c2[nH]c3c1C(=O)NCC3C/C=C(\C)CCNc1cnccc1-2] | (11E)-21-[(3-fluoro-2- methoxyphenyl)amino]-11-methyl- 5,8,16,20- tetraazatetracyclo[16.2.1.0^{2,7}.0^{14,19}]henicosa- 1(21),2,4,6,11,18-hexaen-17-one |
| 16 | 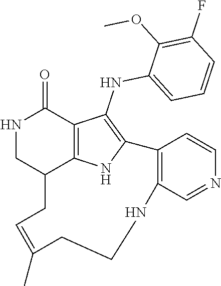 [SMILES: COc1c(F)cccc1Nc1c2[nH]c3c1C(=O)NCC3C/C=C(/C)CCNc1cnccc1-2] | (11Z)-21-[(3-fluoro-2- methoxyphenyl)amino]-11-methyl- 5,8,16,20- tetraazatetracyclo[16.2.1.0^{2,7}.0^{14,19}]henicosa- 1(21),2,4,6,11,18-hexaen-17-one |
| 17 | 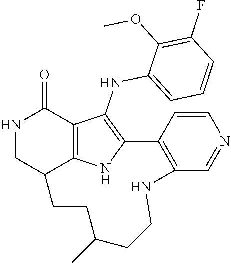 [SMILES: COc1c(F)cccc1Nc1c2[nH]c3c1C(=O)NCC3CCC(C)CCNc1cnccc1-2] | 21-[(3-fluoro-2-methoxyphenyl)amino]-11- methyl-5,8,16,20- tetraazatetracyclo[16.2.1.0^{2,7}.0^{14,19}]henicosa- 1(21),2,4,6,18-pentaen-17-one |
| 18 | 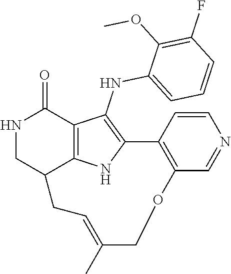 [SMILES: COc1c(F)cccc1Nc1c2[nH]c3c1C(=O)NCC3C/C=C(\C)COc1cnccc1-2] | 20-[(3-fluoro-2-methoxyphenyl)amino]-10- methyl-8-oxa-5,15,19- triazatetracyclo[15.2.1.0^{2,7}.0^{13,18}]icosa- 1(20),2,4,6,10,17-hexaen-16-one |
| 19 | 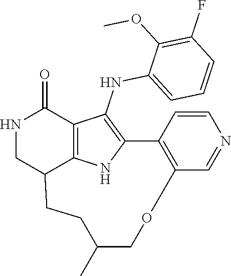 [SMILES: COc1c(F)cccc1Nc1c2[nH]c3c1C(=O)NCC3CCC(C)COc1cnccc1-2] | 20-[(3-fluoro-2-methoxyphenyl)amino]-10- methyl-8-oxa-5,15,19- triazatetracyclo[15.2.1.0^{2,7}.0^{13,18}]icosa- 1(20),2,4,6,17-pentaen-16-one |
| 20 | 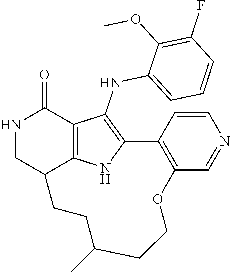 [SMILES: COc1c(F)cccc1Nc1c2[nH]c3c1C(=O)NCC3CCC(C)CCOc1cnccc1-2] | 21-[(3-fluoro-2-methoxyphenyl)amino]-11- methyl-8-oxa-5,16,20- triazatetracyclo[16.2.1.0^{2,7}.0^{14,19}]henicosa- 1(21),2,4,6,18-pentaen-17-one |
| 21 | 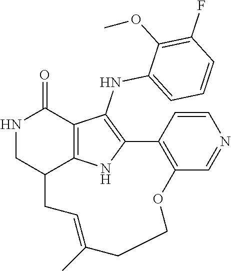 [SMILES: COc1c(F)cccc1Nc1c2[nH]c3c1C(=O)NCC3C/C=C(\C)CCOc1cnccc1-2] | (11E)-21-[(3-fluoro-2- methoxyphenyl)amino]-11-methyl-8-oxa- 5,16,20- triazatetracyclo[16.2.1.0^{2,7}.0^{14,19}]henicosa- 1(21),2,4,6,11,18-hexaen-17-one |
| 22 | 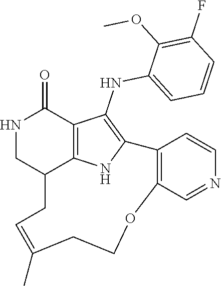 [SMILES: COc1c(F)cccc1Nc1c2[nH]c3c1C(=O)NCC3C/C=C(/C)CCOc1cnccc1-2] | (11Z)-21-[(3-fluoro-2- methoxyphenyl)amino]-11-methyl-8-oxa- 5,16,20- triazatetracyclo[16.2.1.0^{2,7}.0^{14,19}]henicosa- 1(21),2,4,6,11,18-hexaen-17-one |
| 23 | 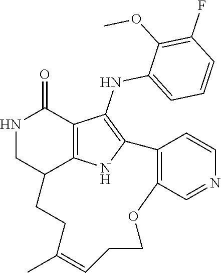 [SMILES: COc1c(F)cccc1Nc1c2[nH]c3c1C(=O)NCC3CC/C(C)=C\CCOc1cnccc1-2] | (11Z)-22-[(3-fluoro-2- methoxyphenyl)amino]-12-methyl-8-oxa- 5,17,21- triazatetracyclo[17.2.1.0^{2,7}.0^{15,20}]docosa- 1(22),2,4,6,11,19-hexaen-18-one |
| 24 | 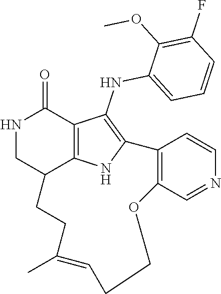 [SMILES: COc1c(F)cccc1Nc1c2[nH]c3c1C(=O)NCC3CC/C(C)=C/CCOc1cnccc1-2] | (11E)-22-[(3-fluoro-2- methoxyphenyl)amino]-12-methyl-8-oxa- 5,17,21- triazatetracyclo[17.2.1.0^{2,7}.0^{15,20}]docosa- 1(22),2,4,6,11,19-hexaen-18-one |
| 25 | 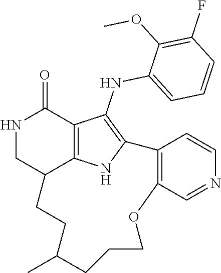 [SMILES: COc1c(F)cccc1Nc1c2[nH]c3c1C(=O)NCC3CCC(C)CCCOc1cnccc1-2] | 22-[(3-fluoro-2-methoxyphenyl)amino]-12- methyl-8-oxa-5,17,21- triazatetracyclo[17.2.1.0^{2,7}.0^{15,20}]docosa- 1(22),2,4,6,19-pentaen-18-one |
| 26 | 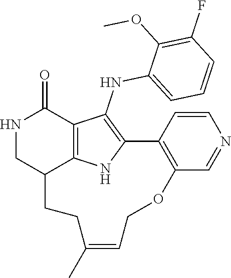 [SMILES: COc1c(F)cccc1Nc1c2[nH]c3c1C(=O)NCC3CC/C(C)=C\COc1cnccc1-2] | (10Z)-21-[(3-fluoro-2- methoxyphenyl)amino]-11-methyl-8-oxa- 5,16,20- triazatetracyclo[16.2.1.0^{2,7}.0^{14,19}]henicosa- 1(21),2,4,6,10,18-hexaen-17-one |
| 27 | 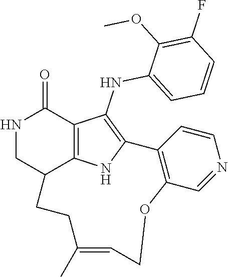 [SMILES: COc1c(F)cccc1Nc1c2[nH]c3c1C(=O)NCC3CC/C(C)=C/COc1cnccc1-2] | (10E)-21-[(3-fluoro-2- methoxyphenyl)amino]-11-methyl-8-oxa- 5,16,20- triazatetracyclo[16.2.1.0^{2,7}.0^{14,19}]henicosa- 1(21),2,4,6,10,18-hexaen-17-one |
| 28 | 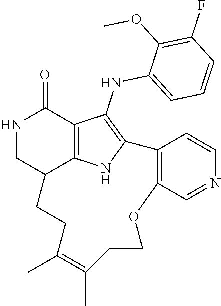 [SMILES: COc1c(F)cccc1Nc1c2[nH]c3c1C(=O)NCC3CC/C(C)=C(/C)CCOc1cnccc1-2] | (11Z)-22-[(3-fluoro-2- methoxyphenyl)amino]-11,12-dimethyl-8- oxa-5,17,21- triazatetracyclo[17.2.1.0^{2,7}.0^{15,20}]docosa- 1(22),2,4,6,11,19-hexaen-18-one |
| 29 | 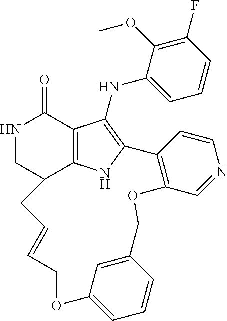 [SMILES: COc1c(F)cccc1Nc1c2[nH]c3c1C(=O)NCC3C/C=C/COc1cccc(c1)COc1cnccc1-2] | (19E)-28-[(3-fluoro-2-methoxyphenyl)amino]- 3,22-dioxa-6,11,15- triazapentacyclo[21.3.1.1^{10,13}.0^{4,9}.0^{12,17}]octacosa- 1(27),4,6,8,10(28),12,19,23,25-nonaen-14-one |
| 30 | 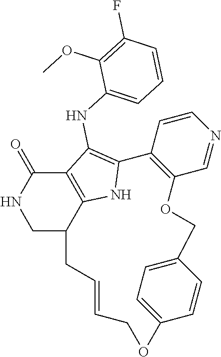 [SMILES: COc1c(F)cccc1Nc1c2[nH]c3c1C(=O)NCC3C/C=C/COc1ccc(cc1)COc1cnccc1-2] | (19E)-28-[(3-fluoro-2-methoxyphenyl)amino]- 3,22-dioxa-6,11,15- triazapentacyclo[21.2.2.1^{10,13}.0^{4,9}.0^{12,17}]octacosa- 1(25),4,6,8,10(28),12,19,23,26-nonaen-14-one |
| 31 | 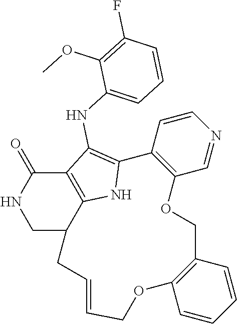 [SMILES: COc1c(F)cccc1Nc1c2[nH]c3c1C(=O)NCC3C/C=C/COc1ccccc1COc1cnccc1-2] | (18E)-28-[(3-fluoro-2-methoxyphenyl)amino]- 8,16-dioxa-5,23,27- triazapentacyclo[23.2.1.0^{2,7}.0^{10,15}.0^{21,26}]octacosa- 1(28),2,4,6,10,12,14,18,25-nonaen-24-one |
| 32 | 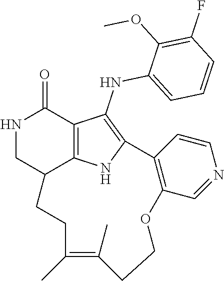 [SMILES: COc1c(F)cccc1Nc1c2[nH]c3c1C(=O)NCC3CC/C(C)=C(\C)CCOc1cnccc1-2] | (11E)-22-[(3-fluoro-2- methoxyphenyl)amino]-11,12-dimethyl-8- oxa-5,17,21- triazatetracyclo[17.2.1.0^{2,7}.0^{15,20}]docosa- 1(22),2,4,6,11,19-hexaen-18-one |
| 33 | 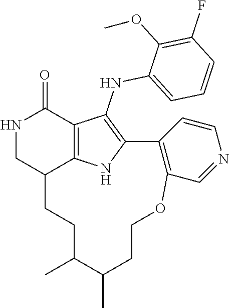 [SMILES: COc1c(F)cccc1Nc1c2[nH]c3c1C(=O)NCC3CCC(C)C(C)CCOc1cnccc1-2] | 22-[(3-fluoro-2-methoxyphenyl)amino]- 11,12-dimethyl-8-oxa-5,17,21- triazatetracyclo[17.2.1.0^{2,7}.0^{15,20}]docosa- 1(22),2,4,6,19-pentaen-18-one |
| 34 | 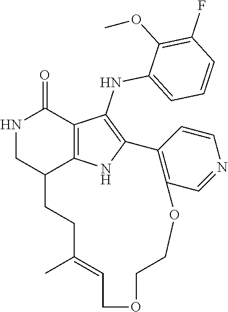 [SMILES: COc1c(F)cccc1Nc1c2[nH]c3c1C(=O)NCC3CC/C(C)=C/COCCOc1cnccc1-2] | (13E)-24-[(3-fluoro-2- methoxyphenyl)amino]-14-methyl-8,11- dioxa-5,19,23- triazatetracyclo[19.2.1.0^{2,7}.0^{17,22}]tetracosa- 1(24),2,4,6,13,21-hexaen-20-one |
| 35 | 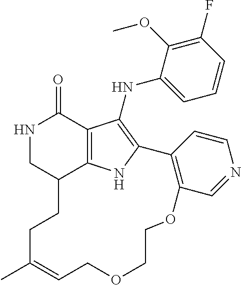 [SMILES: COc1c(F)cccc1Nc1c2[nH]c3c1C(=O)NCC3CC/C(C)=C\COCCOc1cnccc1-2] | (13Z)-24-[(3-fluoro-2- methoxyphenyl)amino]-14-methyl-8,11- dioxa-5,19,23- triazatetracyclo[19.2.1.0^{2,7}.0^{17,22}]tetracosa- 1(24),2,4,6,13,21-hexaen-20-one |
| 36 | 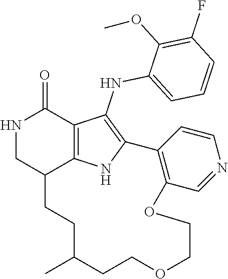 [SMILES: COc1c(F)cccc1Nc1c2[nH]c3c1C(=O)NCC3CCC(C)CCOCCOc1cnccc1-2] | 24-[(3-fluoro-2-methoxyphenyl)amino]-14- methyl-8,11-dioxa-5,19,23- triazatetracyclo[19.2.1.0^{2,7}.0^{17,22}]tetracosa- 1(24),2,4,6,21-pentaen-20-one |
| 37 | 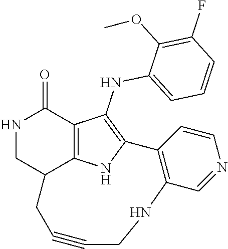 [SMILES: COc1c(F)cccc1Nc1c2[nH]c3c1C(=O)NCC3CC#CCNc1cnccc1-2] | 20-[(3-fluoro-2-methoxyphenyl)amino]- 5,8,15,19- tetraazatetracyclo[15.2.1.0^{2,7}.0^{13,18}]icosa- 1(20),2,4,6,17-pentaen-10-yn-16-one |
| 38 | 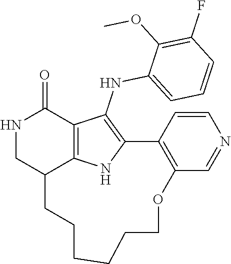 [SMILES: COc1c(F)cccc1Nc1c2[nH]c3c1C(=O)NCC3CCCCCCOc1cnccc1-2] | 22-[(3-fluoro-2-methoxyphenyl)amino]-8- oxa-5,17,21- triazatetracyclo[17.2.1.0^{2,7}.0^{15,20}]docosa- 1(22),2,4,6,19-pentaen-18-one |
| 39 | 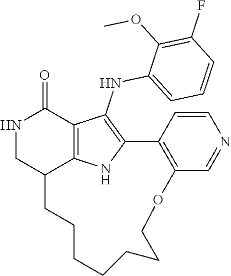 [SMILES: COc1c(F)cccc1Nc1c2[nH]c3c1C(=O)NCC3CCCCCCCOc1cnccc1-2] | 23-[(3-fluoro-2-methoxyphenyl)amino]-8- oxa-5,18,22- triazatetracyclo[18.2.1.0^{2,7}.0^{16,21}]tricosa- 1(23),2,4,6,20-pentaen-19-one |
| 40 | 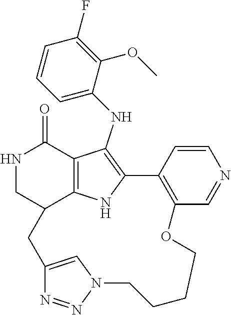 [SMILES: COc1c(F)cccc1Nc1c2[nH]c3c1C(=O)NCC3Cc1cn(nn1)CCCCOc1cnccc1-2] | 25-[(3-fluoro-2-methoxyphenyl)amino]-8- oxa-5,13,14,15,20,24- hexaazapentacyclo[20.2.1.1^{13,16}.0^{2,7}.0^{18.23}]- hexacosa-1(25),2,4,6,14,16(26),22-heptaen- 21-one |
| 41 | 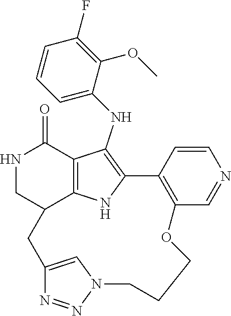 [SMILES: COc1c(F)cccc1Nc1c2[nH]c3c1C(=O)NCC3Cc1cn(nn1)CCCOc1cnccc1-2] | 24-[(3-fluoro-2-methoxyphenyl)amino]-8- oxa-5,12,13,14,19,23- hexaazapentacyclo[19.2.1.1^{12,15}.0^{2,7}.0^{17,22}]- pentacosa-1(24),2,4,6,13,15(25),21- heptaen-20-one |
| 42 | 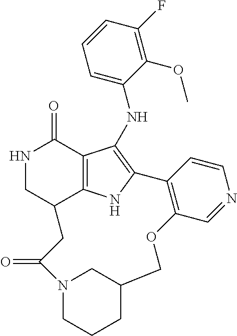 [SMILES: COc1c(F)cccc1Nc1c2[nH]c3c1C(=O)NCC3CC(=O)N1CCCC(COc3cnccc3-2)C1] | 25-[(3-fluoro-2-methoxyphenyl)amino]-18-oxa- 1,6,10,15- tetraazapentacyclo[18.3.1.1^{8,11}.0^{4,9}.0^{12,17}]- pentacosa-8,11(25),12,14,16-pentaene-2,7-dione |
| 43 | 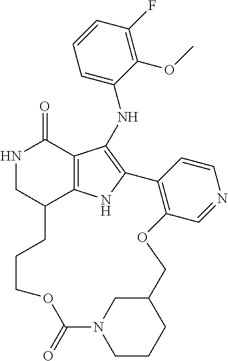 [SMILES: COc1c(F)cccc1Nc1c2[nH]c3c1C(=O)NCC3CCCOC(=O)N1CCCC(COc3cnccc3-2)C1] | 28-[(3-fluoro-2-methoxyphenyl)amino]-3,21- dioxa-6,11,15,23- tetraazapentacyclo[21.3.1.1^{10,13}.0^{4,9}.0^{12,17}]- octacosa-4,6,8,10(28),12-pentaene-14,22-dione |
| 44 | 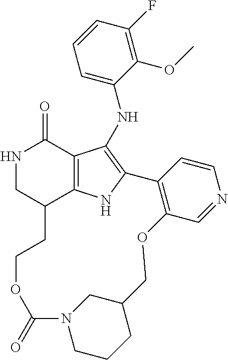 [SMILES: COc1c(F)cccc1Nc1c2[nH]c3c1C(=O)NCC3CCOC(=O)N1CCCC(COc3cnccc3-2)C1] | 27-[(3-fluoro-2-methoxyphenyl)amino]-3,20- dioxa-6,11,15,22- tetraazapentacyclo[20.3.1.1^{10,13}.0^{4,9}.0^{12,17}]- heptacosa-4,6,8,10(27),12-pentaene-14,21-dione |
| 45 | 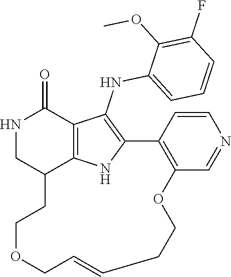 [SMILES: COc1c(F)cccc1Nc1c2[nH]c3c1C(=O)NCC3CCOC/C=C/CCOc1cnccc1-2] | (11E)-24-[(3-fluoro-2- methoxyphenyl)amino]-8,14-dioxa-5,19,23- triazatetracyclo[19.2.1.0^{2,7}.0^{17,22}]tetracosa- 1(24),2,4,6,11,21-hexaen-20-one |
| 46 | 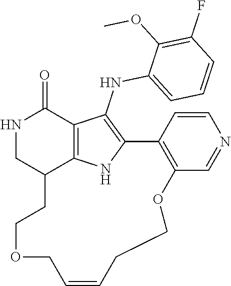 [SMILES: COc1c(F)cccc1Nc1c2[nH]c3c1C(=O)NCC3CCOC/C=C\CCOc1cnccc1-2] | (11Z)-24-[(3-fluoro-2- methoxyphenyl)amino]-8,14-dioxa-5,19,23- triazatetracyclo[19.2.1.0^{2,7}.0^{17,22}]tetracosa- 1(24),2,4,6,11,21-hexaen-20-one |
| 47 | 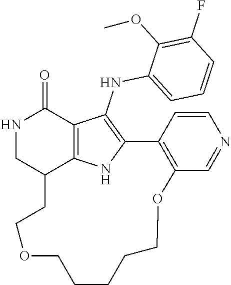 [SMILES: COc1c(F)cccc1Nc1c2[nH]c3c1C(=O)NCC3CCOCCCCCOc1cnccc1-2] | 24-[(3-fluoro-2-methoxyphenyl)amino]- 8,14-dioxa-5,19,23- triazatetracyclo[19.2.1.0^{2,7}.0^{17,22}]tetracosa- 1(24),2,4,6,21-pentaen-20-one |
| 48 | 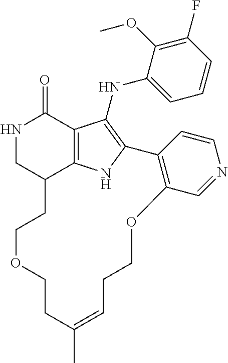 [SMILES: COc1c(F)cccc1Nc1c2[nH]c3c1C(=O)NCC3CCOCC/C(C)=C\CCOc1cnccc1-2] | (11Z)-25-[(3-fluoro-2- methoxyphenyl)amino]-12-methyl-8,15- dioxa-5,20,24- triazatetracyclo[20.2.1.0^{2,7}.0^{18,23}]pentacosa- 1(25),2,4,6,11,22-hexaen-21-one |
| 49 | 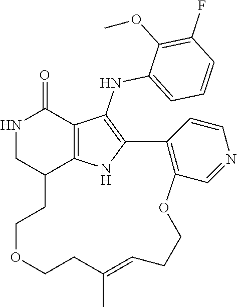 [SMILES: COc1c(F)cccc1Nc1c2[nH]c3c1C(=O)NCC3CCOCC/C(C)=C/CCOc1cnccc1-2] | (11E)-25-[(3-fluoro-2- methoxyphenyl)amino]-12-methyl-8,15- dioxa-5,20,24- triazatetracyclo[20.2.1.0^{2,7}.0^{18,23}]- pentacosa- 1(25)2,4,6,11,22-hexaen-21-one |
| 50 | 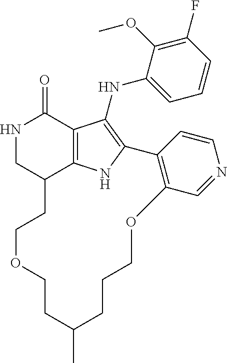 [SMILES: COc1c(F)cccc1Nc1c2[nH]c3c1C(=O)NCC3CCOCCC(C)CCCOc1cnccc1-2] | 25-[(3-fluoro-2-methoxyphenyl)amino]-12- methyl-8,15-dioxa-5,20,24- triazatetracyclo[20.2.1.0^{2,7}.0^{18,23}]pentacosa- 1(25),2,4,6,22-pentaen-21-one |
| 51 | 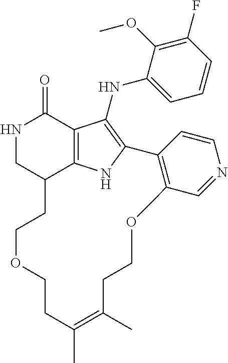 [SMILES: COc1c(F)cccc1Nc1c2[nH]c3c1C(=O)NCC3CCOCC/C(C)=C(/C)CCOc1cnccc1-2] | (11Z)-25-[(3-fluoro-2- methoxyphenyl)amino]-11,12-dimethyl- 8,15-dioxa-5,20,24- triazatetracyclo[20.2.1.0^{2,7}.0^{18,23}]pentacosa- 1(25),2,4,6,11,22-hexaen-21-one |
| 52 | 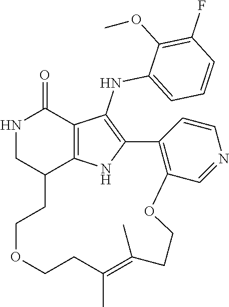 [SMILES: COc1c(F)cccc1Nc1c2[nH]c3c1C(=O)NCC3CCOCC/C(C)=C(\C)CCOc1cnccc1-2] | (11E)-25-[(3-fluoro-2- methoxyphenyl)amino]-11,12-dimethyl- 8,15-dioxa-5,20,24- triazatetracyclo[20.2.1.0^{2,7}.0^{18,23}]pentacosa- 1(25),2,4,6,11,22-hexaen-21-one |
| 53 | 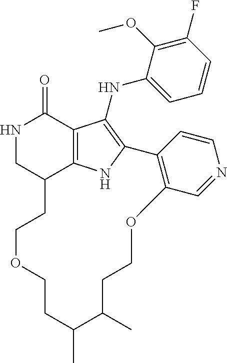 [SMILES: COc1c(F)cccc1Nc1c2[nH]c3c1C(=O)NCC3CCOCCC(C)C(C)CCOc1cnccc1-2] | 25-[(3-fluoro-2-methoxyphenyl)amino]- 11,12-dimethyl-8,15-dioxa-5,20,24- triazatetracyclo[20.2.1.0^{2,7}.0^{18,23}]pentacosa- 1(25),2,4,6,22-pentaen-21-one |
| 54 | 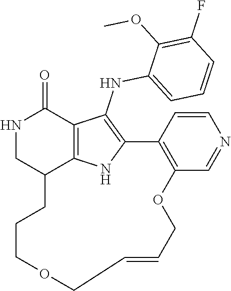 [SMILES: COc1c(F)cccc1Nc1c2[nH]c3c1C(=O)NCC3CCCOC/C=C/COc1cnccc1-2] | (10E)-24-[(3-fluoro-2- methoxyphenyl)amino]-8,13-dioxa-5,19,23- triazatetracyclo[19.2.1.0^{2,7}.0^{17,22}]tetracosa- 1(24),2,4,6,10,21-hexaen-20-one |
| 55 | 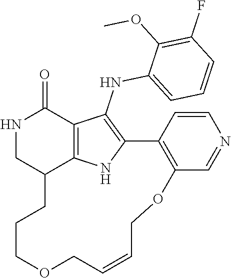 [SMILES: COc1c(F)cccc1Nc1c2[nH]c3c1C(=O)NCC3CCCOC/C=C\COc1cnccc1-2] | (10Z)-24-[(3-fluoro-2- methoxyphenyl)amino]-8,13-dioxa-5,19,23- triazatetracyclo[19.2.1.0^{2,7}.0^{17,22}]tetracosa- 1(24),2,4,6,10,21-hexaen-20-one |
| 56 | 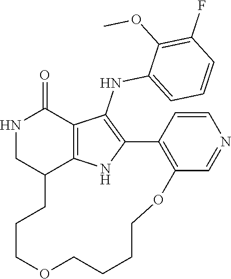 [SMILES: COc1c(F)cccc1Nc1c2[nH]c3c1C(=O)NCC3CCCOCCCCOc1cnccc1-2] | 24-[(3-fluoro-2-methoxyphenyl)amino]- 8,13-dioxa-5,19,23- triazatetracyclo[19.2.1.0^{2,7}.0^{17,22}]tetracosa- 1(24),2,4,6,21-pentaen-20-one |
| 57 | 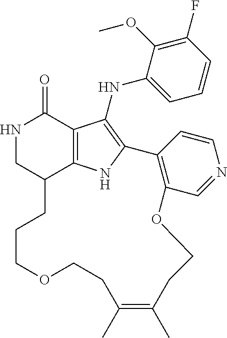 [SMILES: COc1c(F)cccc1Nc1c2[nH]c3c1C(=O)NCC3CCCOCC/C(C)=C(/C)CCOc1cnccc1-2] | (11Z)-26-[(3-fluoro-2- methoxyphenyl)amino]-11,12-dimethyl- 8,15-dioxa-5,21,25- triazatetracyclo[21.2.1.0^{2,7}.0^{19,24}]hexacosa- 1(26),2,4,6,11,23-hexaen-22-one |
| 58 | 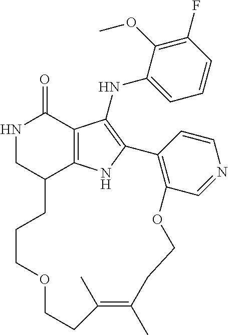 [SMILES: COc1c(F)cccc1Nc1c2[nH]c3c1C(=O)NCC3CCCOCC/C(C)=C(\C)CCOc1cnccc1-2] | (11E)-26-[(3-fluoro-2- methoxyphenyl)amino]-11,12-dimethyl- 8,15-dioxa-5,21,25- triazatetracyclo[21.2.1.0^{2,7}.0^{19,24}]hexacosa- 1(26),2,4,6,11,23-hexaen-22-one |
| 59 | 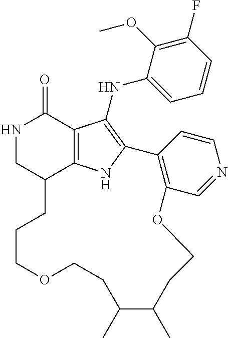 [SMILES: COc1c(F)cccc1Nc1c2[nH]c3c1C(=O)NCC3CCCOCCC(C)C(C)CCOc1cnccc1-2] | 26-[(3-fluoro-2-methoxyphenyl)amino]- 11,12-dimethyl-8,15-dioxa-5,21,25- triazatetracyclo[21.2.1.0^{2,7}.0^{19,24}]hexacosa- 1(26),2,4,6,23-pentaen-22-one |
| 60 | 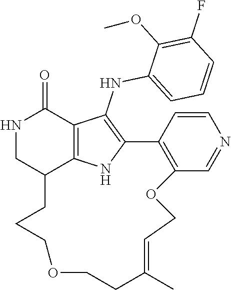 [SMILES: COc1c(F)cccc1Nc1c2[nH]c3c1C(=O)NCC3CCCOCC/C(C)=C/COc1cnccc1-2] | (10E)-25-[(3-fluoro-2- methoxyphenyl)amino]-11-methyl-8,14- dioxa-5,20,24- triazatetracyclo[21.2.1.0^{2,7}.0^{18,23}]pentacosa- 1(25),2,4,6,10,22-hexaen-21-one |
| 61 | 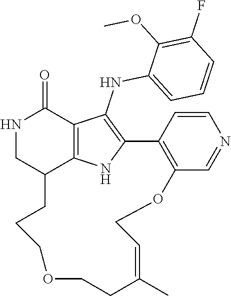 [SMILES: COc1c(F)cccc1Nc1c2[nH]c3c1C(=O)NCC3CCCOCC/C(C)=C\COc1cnccc1-2] | (10Z)-25-[(3-fluoro-2- methoxyphenyl)amino]-11-methyl-8,14- dioxa-5,20,24- triazatetracyclo[20.2.1.0^{2,7}.0^{18,23}]pentacosa- 1(25),2,4,6,10,22-hexaen-21-one |
| 62 | 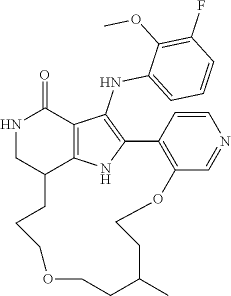 [SMILES: COc1c(F)cccc1Nc1c2[nH]c3c1C(=O)NCC3CCCOCCC(C)CCOc1cnccc1-2] | 25-[(3-fluoro-2-methoxyphenyl)amino]-11- methyl-8,14-dioxa-5,20,24- triazatetracyclo[20.2.1.0^{2,7}.0^{18,23}]pentacosa- 1(25),2,4,6,22-pentaen-21-one |
| 63 | 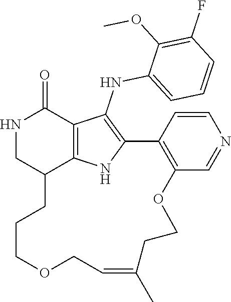 [SMILES: COc1c(F)cccc1Nc1c2[nH]c3c1C(=O)NCC3CCCOC/C=C(/C)CCOc1cnccc1-2] | (11Z)-25-[(3-fluoro-2- methoxyphenyl)amino]-11-methyl-8,14- dioxa-5,20,24- triazatetracyclo[20.2.1.0^{2,7}.0^{18,23}]pentacosa- 1(25),2,4,6,11,22-hexaen-21-one |
| 64 | 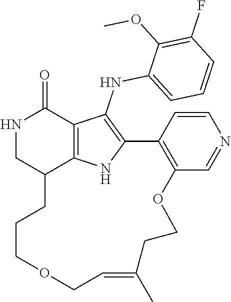 [SMILES: COc1c(F)cccc1Nc1c2[nH]c3c1C(=O)NCC3CCCOC/C=C(\C)CCOc1cnccc1-2] | (11E)-25-[(3-fluoro-2- methoxyphenyl)amino]-11-methyl-8,14- dioxa-5,20,24- triazatetracyclo[20.2.1.0^{2,7}.0^{18,23}]pentacosa- 1(25),2,4,6,11,22-hexaen-21-one |
| 65 | 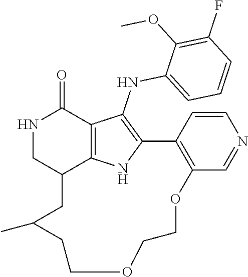 [SMILES: COc1c(F)cccc1Nc1c2[nH]c3c1C(=O)NCC3CC(C)CCOCCOc1cnccc1-2] | 23-[(3-fluoro-2-methoxyphenyl)amino]-14- methyl-8,11-dioxa-5,18,22- triazatetracyclo[18.2.1.0^{2,7}.0^{16,21}]tricosa- 1(23),2,4,6,20-pentaen-19-one |
| 66 | 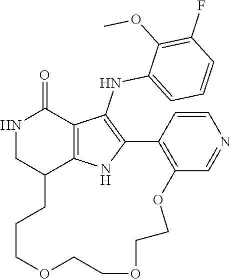 [SMILES: COc1c(F)cccc1Nc1c2[nH]c3c1C(=O)NCC3CCCOCCOCCOc1cnccc1-2] | 25-[(3-fluoro-2-methoxyphenyl)amino]- 8,11,14-trioxa-5,20,24- triazatetracyclo[20.2.1.0^{2,7}.0^{18,23}]pentacosa- 1(25),2,4,6,22-pentaen-21-one |
| 67 | 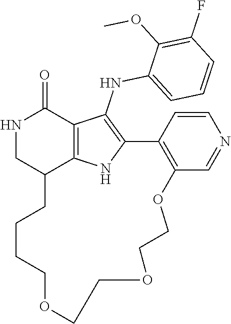 [SMILES: COc1c(F)cccc1Nc1c2[nH]c3c1C(=O)NCC3CCCCOCCOCCOc1cnccc1-2] | 26-[(3-fluoro-2-methoxyphenyl)amino]- 8,11,14-trioxa-5,21,25- triazatetracyclo[21.2.1.0^{2,7}.0^{19,24}]hexacosa- 1(26),2,4,6,23-pentaen-22-one |
| 68 | 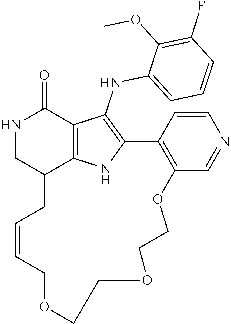 [SMILES: COc1c(F)cccc1Nc1c2[nH]c3c1C(=O)NCC3C/C=C\COCCOCCOc1cnccc1-2] | (16Z)-26-[(3-fluoro-2- methoxyphenyl)amino]-8,11,14-trioxa- 5,21,25- triazatetracyclo[21.2.1.0^{2,7}.0^{19,24}]hexacosa- 1(26),2,4,6,16,23-hexaen-22-one |
| 69 | 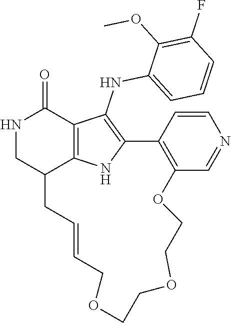 [SMILES: COc1c(F)cccc1Nc1c2[nH]c3c1C(=O)NCC3C/C=C/COCCOCCOc1cnccc1-2] | (16E)-26-[(3-fluoro-2- methoxyphenyl)amino]-8,11,14-trioxa- 5,21,25- triazatetracyclo[21.2.1.0^{2,7}.0^{19,24}]hexacosa- 1(26),2,4,6,16,23-hexaen-22-one |
| 70 | 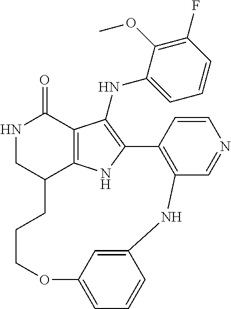 [SMILES: COc1c(F)cccc1Nc1c2[nH]c3c1C(=O)NCC3CCCOc1cccc(c1)Nc1cnccc1-2] | 26-[(3-fluoro-2-methoxyphenyl)amino]-20-oxa- 2,5,10,14- tetraazapentacyclo[19.3.1.1^{9,12}.0^{3,8}.0^{11,16}]- hexacosa-1(25),3,5,7,9(26),11,21,23-octaen-13- one |
| 71 | 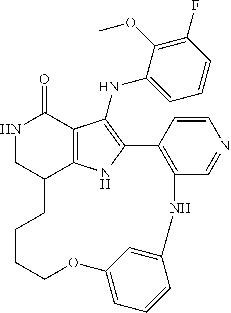 [SMILES: COc1c(F)cccc1Nc1c2[nH]c3c1C(=O)NCC3CCCCOc1cccc(c1)Nc1cnccc1-2] | 27-[(3-fluoro-2-methoxyphenyl)amino]-21-oxa- 2,5,10,14- tetraazapentacyclo[20.3.1.1^{9,12}.0^{3,8}.0^{11,16}]- heptacosa-1(26),3,5,7,9(27),11,22,24-octaen-13- one |
| 72 | 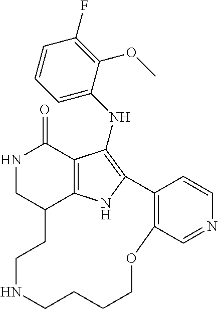 [SMILES: COc1c(F)cccc1Nc1c2[nH]c3c1C(=O)NCC3CCNCCCCOc1cnccc1-2] | 23-[(3-fluoro-2-methoxyphenyl)amino]-8- oxa-5,13,18,22- tetraazatetracyclo[18.2.1.0^{2,7}.0^{16,21}]tricosa- 1(23),2,4,6,20-pentaen-19-one |
| 73 | 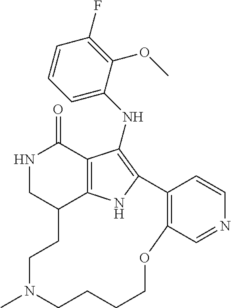 [SMILES: COc1c(F)cccc1Nc1c2[nH]c3c1C(=O)NCC3CCN(C)CCCCOc1cnccc1-2] | 23-[(3-fluoro-2-methoxyphenyl)amino]-13- methyl-8-oxa-5,13,18,22- tetraazatetracyclo[18.2.1.0^{2,7}.0^{16,21}]tricosa- 1(23),2,4,6,20-pentaen-19-one |
| 74 | 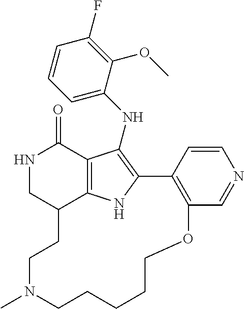 [SMILES: COc1c(F)cccc1Nc1c2[nH]c3c1C(=O)NCC3CCN(C)CCCCCOc1cnccc1-2] | 24-[(3-fluoro-2-methoxyphenyl)amino]-14- methyl-8-oxa-5,14,19,23- tetraazatetracyclo[19.2.1.0^{2,7}.0^{17,22}]tetracosa- 1(24),2,4,6,21-pentaen-20-one |
| 75 | 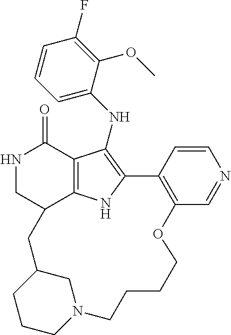 [SMILES: COc1c(F)cccc1Nc1c2[nH]c3c1C(=O)NCC3CC1CCCN(CCCCOc3cnccc3-2)C1] | 27-[(3-fluoro-2-methoxyphenyl)amino]-17- oxa-5,9,14,22- tetraazapentacyclo[20.3.1.1^{7,10}.0^{3,8}.0^{11,16}]- heptacosa-7,10(27),11,13,15-pentaen-6-one |
| 76 | 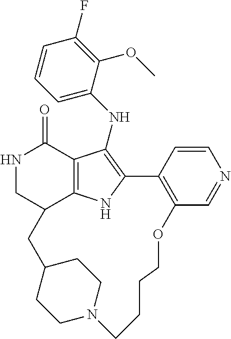 [SMILES: COc1c(F)cccc1Nc1c2[nH]c3c1C(=O)NCC3CC1CCN(CCCCOc3cnccc3-2)CC1] | 27-[(3-fluoro-2-methoxyphenyl)amino]-17- oxa-5,9,14,22- tetraazapentacyclo[20.2.2.1^{7,10}.0^{3,8}.0^{11,16}]- heptacosa-7,10(27),11,13,15-pentaen-6-one |
| 77 | 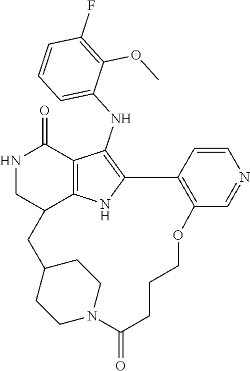 [SMILES: COc1c(F)cccc1Nc1c2[nH]c3c1C(=O)NCC3CC1CCN(CC1)C(=O)CCCOc1cnccc1-2] | 27-[(3-fluoro-2-methoxyphenyl)amino]-17- oxa-5,9,14,22- tetraazapentacyclo[20.2.2.1^{7,10}.0^{3,8}.0^{11,16}]- heptacosa-7,10(27),11,13,15-pentaene-6,21- dione |
| 78 | 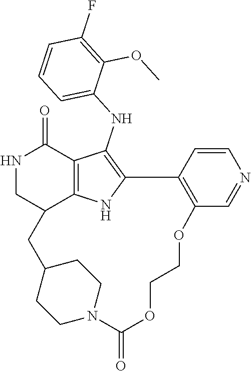 [SMILES: COc1c(F)cccc1Nc1c2[nH]c3c1C(=O)NCC3CC1CCN(CC1)C(=O)OCCOc1cnccc1-2] | 27-[(3-fluoro-2-methoxyphenyl)amino]- 17,20-dioxa-5,9,14,22- tetraazapentacyclo[20.2.2.1^{7,10}.0^{3,8}.0^{11,16}]- heptacosa-7,10(27),11,13,15-pentaene-6,21- dione |
| 79 | 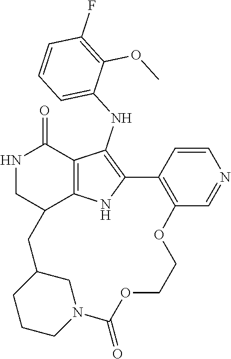 [SMILES: COc1c(F)cccc1Nc1c2[nH]c3c1C(=O)NCC3CC1CCCN(C1)C(=O)OCCOc1cnccc1-2] | 27-[(3-fluoro-2-methoxyphenyl)amino]- 17,20-dioxa-5,9,14,22- tetraazapentacyclo[20.3.1.1^{7,10}.0^{3,8}.0^{11,16}]- heptacosa-7,10(27),11,13,15-pentaene-6,21- dione |
| 80 | 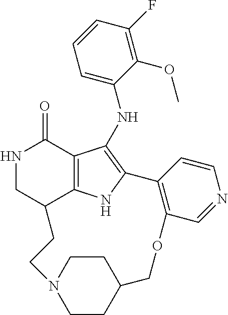 [SMILES: COc1c(F)cccc1Nc1c2[nH]c3c1C(=O)NCC3CCN1CCC(CC1)COc1cnccc1-2] | 25-[(3-fluoro-2-methoxyphenyl)amino]-18-oxa- 1,6,10,15-tetraazapentacyclo [18.2.2.1^{8,11}.0^{4,9}.0^{12,17}]pentacosa- 8,11(25),12,14,16-pentaen-7-one |
| 81 | 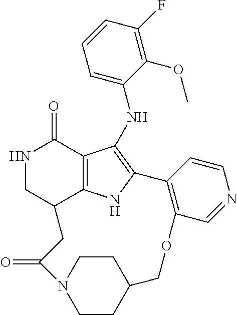 [SMILES: COc1c(F)cccc1Nc1c2[nH]c3c1C(=O)NCC3CC(=O)N1CCC(CC1)COc1cnccc1-2] | 25-[(3-fluoro-2-methoxyphenyl)amino]-18-oxa- 1,6,10,15-tetraazapentacyclo [18.2.2.1^{8,11}.0^{4,9}.0^{12,17}]pentacosa- 8,11(25),12,14,16-pentaene-2,7-dione |
| 82 | 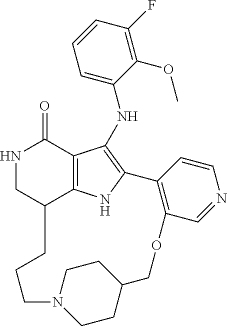 [SMILES: COc1c(F)cccc1Nc1c2[nH]c3c1C(=O)NCC3CCCN1CCC(CC1)COc1cnccc1-2] | 26-[(3-fluoro-2-methoxyphenyl)amino]-3-oxa- 6,11,15,21- tetraazapentacyclo[19.2.2.1^{10,13}.0^{4,9}.0^{12,17}]- hexacosa-4,6,8,10(26),12-pentaen-14-one |
| 83 | 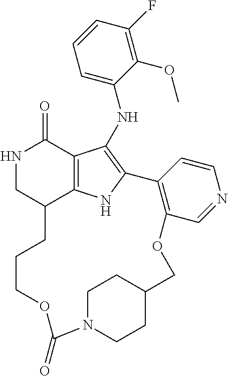 [SMILES: COc1c(F)cccc1Nc1c2[nH]c3c1C(=O)NCC3CCCOC(=O)N1CCC(CC1)COc1cnccc1-2] | 28-[(3-fluoro-2-methoxyphenyl)amino]-3,21- dioxa-6,11,15,23- tetraazapentacyclo[21.2.2.1^{10,13}.0^{4,9}.0^{12,17}]- octacosa-4,6,8,10(28),12-pentaene-14,22-dione |
| 84 | 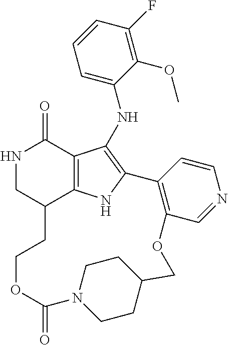 [SMILES: COc1c(F)cccc1Nc1c2[nH]c3c1C(=O)NCC3CCOC(=O)N1CCC(CC1)COc1cnccc1-2] | 27-[(3-fluoro-2-methoxyphenyl)amino]-3,20- dioxa-6,11,15,22- tetraazapentacyclo[20.2.2.1^{10,13}.0^{4,9}.0^{12,17}]- heptacosa-4,6,8,10(27),12-pentaene-14,21-dione |
| 85 | 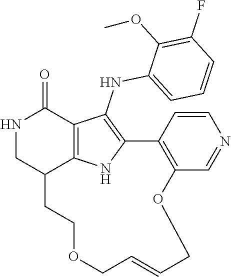 [SMILES: COc1c(F)cccc1Nc1c2[nH]c3c1C(=O)NCC3CCOC/C=C/COc1cnccc1-2] | (10E)-23-[(3-fluoro-2- methoxyphenyl)amino]-8,13-dioxa-5,18,22- triazatetracyclo[18.2.1.0^{2,7}.0^{16,21}]tricosa- 1(23),2,4,6,10,20-hexaen-19-one |
| 86 | 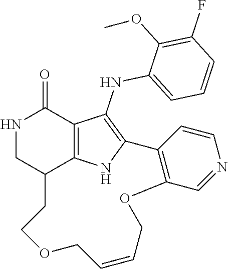 [SMILES: COc1c(F)cccc1Nc1c2[nH]c3c1C(=O)NCC3CCOC/C=C\COc1cnccc1-2] | (10Z)-23-[(3-fluoro-2- methoxyphenyl)amino]-8,13-dioxa-5,18,22- triazatetracyclo[18.2.1.0^{2,7}.0^{16,21}]tricosa- 1(23),2,4,6,10,20-hexaen-19-one |
| 87 | 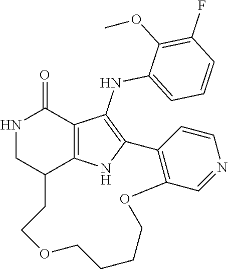 [SMILES: COc1c(F)cccc1Nc1c2[nH]c3c1C(=O)NCC3CCOCCCCOc1cnccc1-2] | 23-[(3-fluoro-2-methoxyphenyl)amino]- 8,13-dioxa-5,18,22- triazatetracyclo[18.2.1.02,7.016,21]tricosa- 1(23),2,4,6,20-pentaen-19-one |
| 88 | 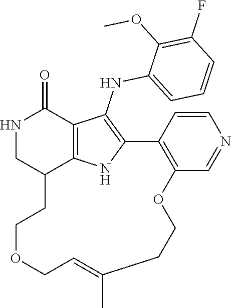 [SMILES: COc1c(F)cccc1Nc1c2[nH]c3c1C(=O)NCC3CCOC/C=C(\C)CCOc1cnccc1-2] | (11E)-24-[(3-fluoro-2- methoxyphenyl)amino]-11-methyl-8,14- dioxa-5,19,23- triazatetracyclo[19.2.1.0^{2,7}.0^{17,22}]tetracosa- 1(24),2,4,6,11,21-hexaen-20-one |
| 89 | 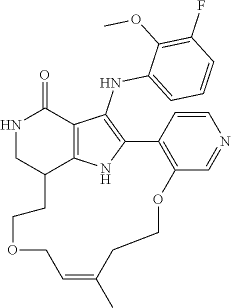 [SMILES: COc1c(F)cccc1Nc1c2[nH]c3c1C(=O)NCC3CCOC/C=C(/C)CCOc1cnccc1-2] | (11Z)-24-[(3-fluoro-2- methoxyphenyl)amino]-11-methyl-8,14- dioxa-5,19,23- triazatetracyclo[19.2.1.0^{2,7}.0^{17,22}]tetracosa- 1(24),2,4,6,11,21-hexaen-20-one |
| 90 | 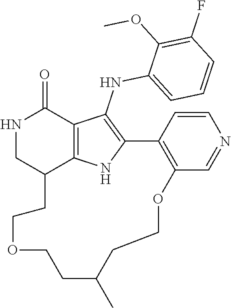 [SMILES: COc1c(F)cccc1Nc1c2[nH]c3c1C(=O)NCC3CCOCCC(C)CCOc1cnccc1-2] | 24-[(3-fluoro-2-methoxyphenyl)amino]-11- methyl-8,14-dioxa-5,19,23- triazatetracyclo[19.2.1.0^{2,7}.0^{17,22}]tetracosa- 1(24),2,4,6,21-pentaen-20-one |
| 91 | 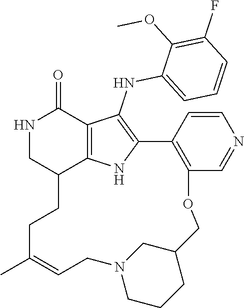 [SMILES: COc1c(F)cccc1Nc1c2[nH]c3c1C(=O)NCC3CC/C(C)=C\CN1CCCC(COc3cnccc3-2)C1] | (20Z)-28-[(3-fluoro-2-methoxyphenyl)amino]- 20-methyl-3-oxa-6,11,15,23- tetraazapentacyclo[21.3.1.1^{10,13}.0^{4,9}.0^{12,17}]- octacosa-4,6,8,10(28),12,20-hexaen-14-one |
| 92 | 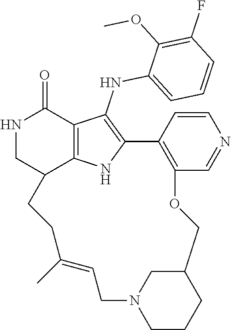 [SMILES: COc1c(F)cccc1Nc1c2[nH]c3c1C(=O)NCC3CC/C(C)=C/CN1CCCC(COc3cnccc3-2)C1] | (20E)-28-[(3-fluoro-2-methoxyphenyl)amino]- 20-methyl-3-ox-6,11,15,23- tetraazapentacyclo[21.3.1.1^{10,13}.0^{4,9}.0^{12,17}]- octacosa-4,6,8,10(28),12,20-hexaen-14-one |
| 93 | 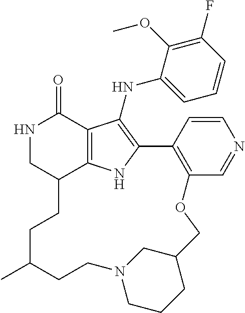 [SMILES: COc1c(F)cccc1Nc1c2[nH]c3c1C(=O)NCC3CCC(C)CCN1CCCC(COc3cnccc3-2)C1] | 28-[(3-fluoro-2-methoxyphenyl)amino]-20- methyl-3-oxa-6,11,15,23- tetraazapentacyclo[21.3.1.1^{10,13}.0^{4,9}.0^{12,17}]- octacosa-4,6,8,10(28),12-pentaen-14-one |
| 94 | 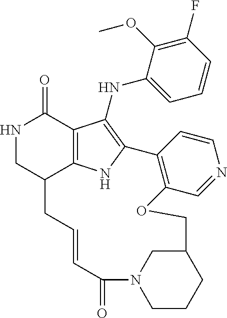 [SMILES: COc1c(F)cccc1Nc1c2[nH]c3c1C(=O)NCC3C/C=C/C(=O)N1CCCC(COc3cnccc3-2)C1] | 27-[(3-fluoro-2-methoxyphenyl)amino]-3-oxa- 6,11,15,22- tetraazapentacyclo[20.3.1.1^{10,13}.0^{4,9}.0^{12,17}]- heptacosa-4,6,8,10(27),12,19-hexaene-14,21- dione |
| 95 | 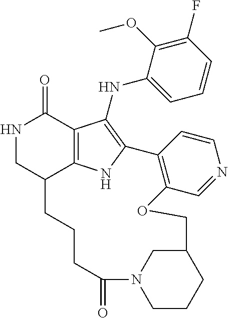 [SMILES: COc1c(F)cccc1Nc1c2[nH]c3c1C(=O)NCC3CCCC(=O)N1CCCC(COc3cnccc3-2)C1] | 27-[(3-fluoro-2-methoxyphenyl)amino]-3-oxa- 6,11,15,22- tetraazapentacyclo[20.3.1.1^{10,13}.0^{4,9}.0^{12,17}]- heptacosa-4,6,8,10(27),12-pentaene-14,21-dione |
| 96 | 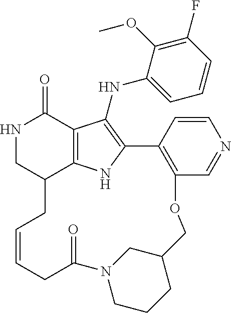 [SMILES: COc1c(F)cccc1Nc1c2[nH]c3c1C(=O)NCC3C/C=C\CC(=O)N1CCCC(COc3cnccc3-2)C1] | (19Z)-28-[(3-fluoro-2-methoxyphenyl)amino]-3- oxa-6,11,15,23- tetraazapentacyclo[21.3.1.1^{10,13}.0^{4,9}.0^{12,17}]- octacosa-4,6,8,10(28),12,19-hexaene-14,22- dione |
| 97 | 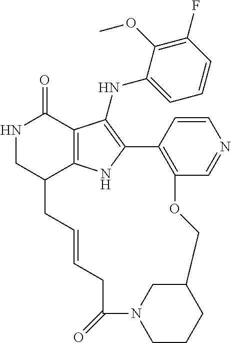 [SMILES: COc1c(F)cccc1Nc1c2[nH]c3c1C(=O)NCC3C/C=C/CC(=O)N1CCCC(COc3cnccc3-2)C1] | (19E)-28-[(3-fluoro-2-methoxyphenyl)amino]-3- oxa-6,11,15,23- tetraazapentacyclo[21.3.1.1^{10,13}.0^{4,9}.0^{12,17}]- octacosa-4,6,8,10(28),12,19-hexaene-14,22- dione |
| 98 | 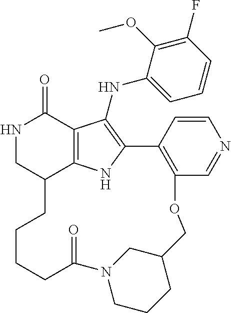 [SMILES: COc1c(F)cccc1Nc1c2[nH]c3c1C(=O)NCC3CCCCC(=O)N1CCCC(COc3cnccc3-2)C1] | 28-[(3-fluoro-2-methoxyphenyl)amino]-3-oxa- 6,11,15,23- tetraazapentacyclo[21.3.1.1^{10,13}.0^{4,9}.0^{12,17}]- octacosa-4,6,8,10(28),12-pentaene-14,22-dione |
| 99 | 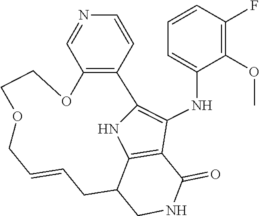 [SMILES: COc1c(F)cccc1Nc1c2[nH]c3c1C(=O)NCC3C/C=C/COCCOc1cnccc1-2] | (13E)-23-[(3-fluoro-2- methoxyphenyl)amino]-8,11-dioxa-5,18,22- triazatetracyclo[18.2.1.0^{2,7}.0^{16,21}]tricosa- 1(23),2,4,6,13,20-hexaen-19-one |
| 100 | 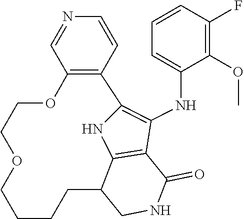 [SMILES: COc1c(F)cccc1Nc1c2[nH]c3c1C(=O)NCC3CCCCOCCOc1cnccc1-2] | 23-[(3-fluoro-2-methoxyphenyl)amino]- 8,11-dioxa-5,18,22- triazatetracyclo[18.2.1.0^{2,7}.0^{16,21}]tricosa- 1(23),2,4,6,20-pentaen-19-one |
| 101 | 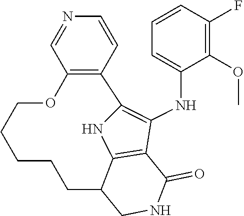 [SMILES: COc1c(F)cccc1Nc1c2[nH]c3c1C(=O)NCC3CCCCCOc1cnccc1-2] | 21-[(3-fluoro-2-methoxyphenyl)amino]-8- oxa-5,16,20- triazatetracyclo[16.2.1.0^{2,7}.0^{14,19}]henicosa- 1(21),2,4,6,18-pentaen-17-one |
| 102 | 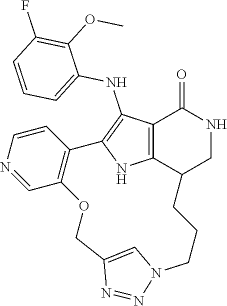 [SMILES: COc1c(F)cccc1Nc1c2[nH]c3c1C(=O)NCC3CCCn1cc(nn1)COc1cnccc1-2] | 24-[(3-fluoro-2-methoxyphenyl)amino]-8-oxa- 5,11,12,13,19,23- hexaazapentacyclo[19.2.1.1^{10,13}.0^{2,7}.0^{17,22}]- pentacosa-1(24),2,4,6,10(25),11,21-heptaen-20- one |
| 103 | 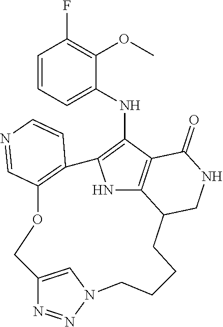 [SMILES: COc1c(F)cccc1Nc1c2[nH]c3c1C(=O)NCC3CCCCn1cc(nn1)COc1cnccc1-2] | 25-[(3-fluoro-2-methoxyphenyl)amino]-8-oxa- 5,11,12,13,20,24- hexaazapentacyclo[20.2.1.1^{10,13}.0^{2,7}.0^{18,23}]- hexacosa-1(25),2,4,6,10(26),11,22-heptaen-21- one |
| 104 | 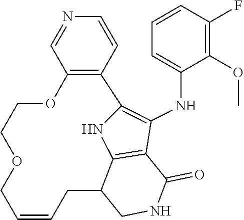 [SMILES: COc1c(F)cccc1Nc1c2[nH]c3c1C(=O)NCC3C/C=C\COCCOc1cnccc1-2] | (13Z)-23-[(3-fluoro-2- methoxyphenyl)amino]-8,11-dioxa-5,18,22- triazatetracyclo[18.2.1.0^{2,7}.0^{16,21}]tricosa- 1(23),2,4,6,13,20-hexaen-19-one |
| 105 | 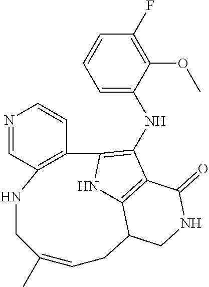 [SMILES: COc1c(F)cccc1Nc1c2[nH]c3c1C(=O)NCC3C/C=C(/C)CNc1cnccc1-2] | 20-[(3-fluoro-2-methoxyphenyl)amino]-10- methyl-5,8,15,19- tetraazatetracyclo[15.2.1.0^{2,7}.0^{13,18}]icosa- 1(20),2,4,6,10,17-hexaen-16-one |
| 106 | 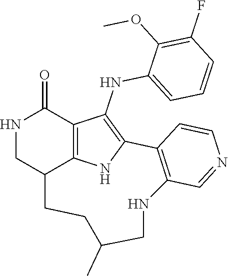 [SMILES: COc1c(F)cccc1Nc1c2[nH]c3c1C(=O)NCC3CCC(C)CNc1cnccc1-2] | 20-[(3-fluoro-2-methoxyphenyl)amino]-10- methyl-5,8,15,19- tetraazatetracyclo[15.2.1.0^{2,7}.0^{13,18}]icosa- 1(20),2,4,6,17-pentaen-16-one |
| 107 | 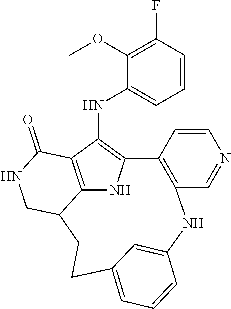 [SMILES: COc1c(F)cccc1Nc1c2[nH]c3c1C(=O)NCC3CCc1cccc(c1)Nc1cnccc1-2] | 24-[(3-fluoro-2-methoxyphenyl)amino]- 2,5,10,14- tetraazapentacyclo[17.3.1.1^{9,12}.0^{3,8}.0^{11,16}]- tetracosa-1(23),3,5,7,9(24),11,19,21-octaen- 13-one |
| 108 | 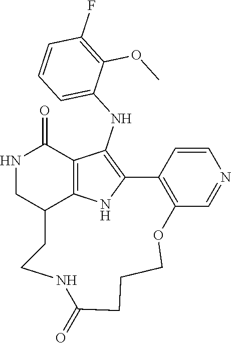 [SMILES: COc1c(F)cccc1Nc1c2[nH]c3c1C(=O)NCC3CCNC(=O)CCCOc1cnccc1-2] | 23-[(3-fluoro-2-methoxyphenyl)amino]-8- oxa-5,13,18,22- tetraazatetracyclo[18.2.1.0^{2,7}.0^{16,21}]tricosa- 1(23),2,4,6,20-pentaen-12,19-dione |
| 109 | 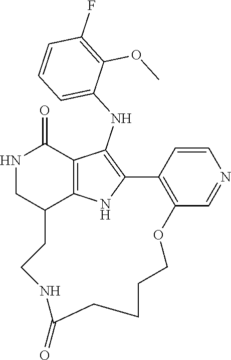 [SMILES: COc1c(F)cccc1Nc1c2[nH]c3c1C(=O)NCC3CCNC(=O)CCCCOc1cnccc1-2] | 24-[(3-fluoro-2-methoxyphenyl)amino]-8- oxa-5,14,19,23- tetraazatetracyclo[19.2.1.0^{2,7}.0^{17,22}]tetracosa- 1(24),2,4,6,21-pentaen-13,20-dione |
| 110 | 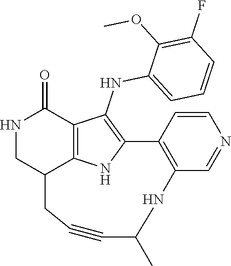 [SMILES: COc1c(F)cccc1Nc1c2[nH]c3c1C(=O)NCC3CC#CC(C)Nc1cnccc1-2] | 20-[(3-fluoro-2-methoxyphenyl)amino]-9- methyl-5,8,15,19- tetraazatetracyclo[15.2.1.0^{2,7}.0^{13,18}]icosa- 1(20),2,4,6,17-pentaen-10-yn-16-one |
| 111 | 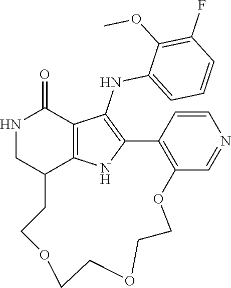 [SMILES: COc1c(F)cccc1Nc1c2[nH]c3c1C(=O)NCC3CCOCCOCCOc1cnccc1-2] | 24-[(3-fluoro-2-methoxyphenyl)amino]- 8,11,14-trioxa-5,19,23- triazatetracyclo[19.2.1.0^{2,7}.0^{17,22}]tetracosa- 1(24),2,4,6,21-pentaen-20-one |
| 112 | 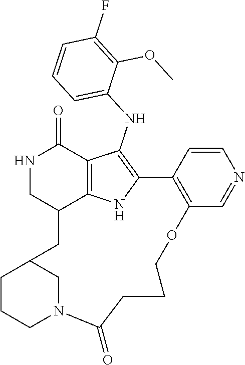 [SMILES: COc1c(F)cccc1Nc1c2[nH]c3c1C(=O)NCC3CC1CCCN(C1)C(=O)CCCOc1cnccc1-2] | 27-[(3-fluoro-2-methoxyphenyl)amino]-17- oxa-5,9,14,22- tetraazapentacyclo[20.3.1.1^{7,10}.0^{3,8}.0^{11,16}]- heptacosa-7,10(27),11,13,15-pentaene-6,21- dione |
| 113 | 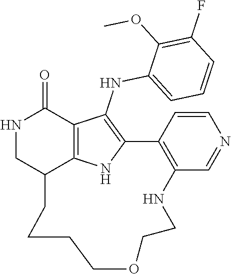 [SMILES: COc1c(F)cccc1Nc1c2[nH]c3c1C(=O)NCC3CCCCOCCNc1cnccc1-2] | 23-[(3-fluoro-2-methoxyphenyl)amino]-11- oxa-5,8,18,22- tetraazatetracyclo[18.2.1.0^{7,10}.0^{16,21}]tricosa- 1(23),2,4,6,20-pentaen-19-one |
| 114 | 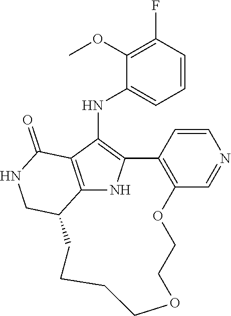 [SMILES: COc1c(F)cccc1Nc1c2[nH]c3c1C(=O)NC[C@H]3CCCCOCCOc1cnccc1-2] | (16R)-23-[(3-fluoro-2- methoxyphenyl)amino]-8,11-dioxa-5,18,22- triazatetracyclo[18.2.1.0^{2,7}.0^{16,21}]tricosa- 1(23),2,4,6,20-pentaen-19-one |
| 115 | 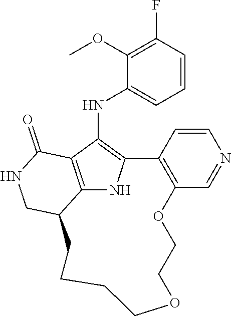 [SMILES: COc1c(F)cccc1Nc1c2[nH]c3c1C(=O)NC[C@@H]3CCCCOCCOc1cnccc1-2] | (16S)-23-[(3-fluoro-2- methoxyphenyl)amino]-8,11-dioxa-5,18,22- triazatetracyclo[18.2.1.0^{2,7}.0^{16,21}]tricosa- 1(23),2,4,6,20-pentaen-19-one |
| 116 | 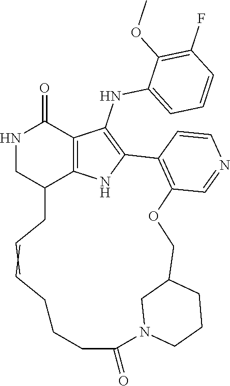 [SMILES: COc1c(F)cccc1Nc1c2[nH]c3c1C(=O)NCC3CC=CCCCC(=O)N1CCCC(COc3cnccc3-2)C1] | 25-[(3-fluoro-2-methoxyphenyl)amino]- 7,8,9,10,14,15,18,18a,19,20-decahydro-6H-7,11- methano-2,24-methenodipyrido[3,4-b:4′,3′- f][1,5,15]oxadiazacycloicosine-12,21(13H,23H)- dione |
| 117 | 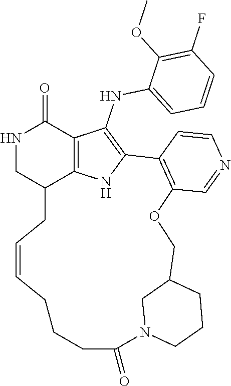 [SMILES: COc1c(F)cccc1Nc1c2[nH]c3c1C(=O)NCC3C/C=C\CCCC(=O)N1CCCC(COc3cnccc3-2)C1] | Z-25-[(3-fluoro-2-methoxyphenyl)amino]- 7,8,9,10,14,15,18,18a,19,20-decahydro-6H-7,11- methano-22,24-methenopyrido[3,4-b:4′,3′- f][1,5,15]oxadiazacycloicosine-12,21(13H,23H)- dione |
| 118 | 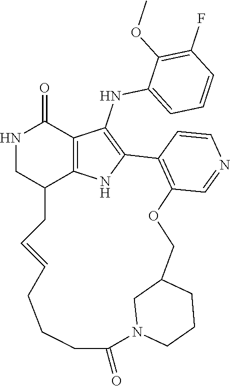 [SMILES: COc1c(F)cccc1Nc1c2[nH]c3c1C(=O)NCC3C/C=C/CCCC(=O)N1CCCC(COc3cnccc3-2)C1] | E-25-[(3-fluoro-2-methoxyphenyl)amino]- 7,8,9,10,14,15,18,18a,19,20-decahydro-6H-7,11- methano-22,24-methenodipyrido[3,4-b:4′,3′- f][1,5,15]oxadiazacycloicosine-12,21(13H,23H)- dione |
| 119 | 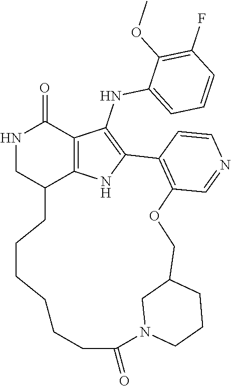 [SMILES: COc1c(F)cccc1Nc1c2[nH]c3c1C(=O)NCC3CCCCCCC(=O)N1CCCC(COc3cnccc3-2)C1] | 25-[(3-fluoro-2-methoxyphenyl)amino]- 7,8,9,10,14,15,16,17,18,18a,19,20-dodecahydro- 6H-7,11-methano-22,24-methenodipyrido[3,4- b:4′,3′-f][1,5,15]oxadiazacycloicosine- 12,21(13H,23H)-dione |
| 120 | 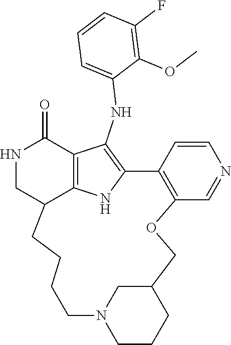 [SMILES: COc1c(F)cccc1Nc1c2[nH]c3c1C(=O)NCC3CCCCN1CCCC(COc3cnccc3-2)C1] | 22-(3-fluoro-2-methoxyanilino)- 7,8,9,10,13,14,15,15a,16,17-decahydro-6H,12H- 7,11-methano-9,21-(metheno)dipyrido[3,4- b:4′,3′-f][1,5,12]oxadiazacycloheptadecin- 18(20H)-one |
| 121 | 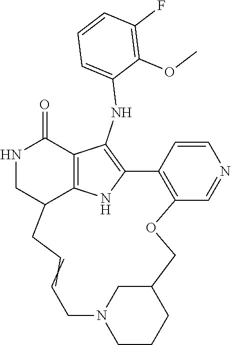 [SMILES: COc1c(F)cccc1Nc1c2[nH]c3c1C(=O)NCC3CC=CCN1CCCC(COc3cnccc3-2)C1] | 22-(3-fluoro-2-methoxyanilino)- 7,8,9,10,15,15a,16,17-octahydro-6H,12H-7,11- methano-19,21-(metheno)dipyrido[3,4-b:4′,3′- f][1,5,12]oxadiazacycloheptadecin-18(20H)-one |
| 122 | 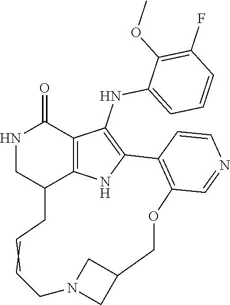 [SMILES: COc1c(F)cccc1Nc1c2[nH]c3c1C(=O)NCC3CC=CCN1CC(COc3cnccc3-2)C1] | 20-(3-fluoro-2-methoxyanilino)- 7,8,13,13a,14,15-hexahydro-6H,10H-7,9- methano-17,19-(metheno)dipyrido[3,4-b:4′,3′- f][1,5,12]oxadiazacyclopentadecin-16(18H)-one |
| 123 | 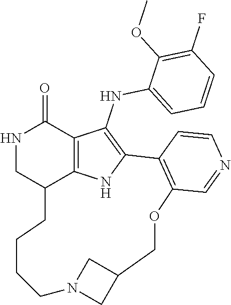 [SMILES: COc1c(F)cccc1Nc1c2[nH]c3c1C(=O)NCC3CCCCN1CC(COc3cnccc3-2)C1] | 20-(3-fluoro-2-methoxyanilino)- 7,8,11,12,13,13a,14,15-octahydro-6H,10H-7,9- methano-17,19-(metheno)dipyrido[3,4-b:4′,3′- f][1,5,12]oxadiazacyclopentadecin-16(18H)-one |
| 124 | 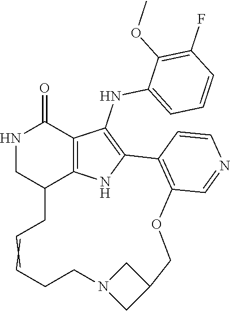 [SMILES: COc1c(F)cccc1Nc1c2[nH]c3c1C(=O)NCC3CC=CCCN1CC(COc3cnccc3-2)C1] | 21-(3-fluoro-2-methoxyanilino)- 7,8,10,11,14,14a,15,16-octahydro-6H-7,9- methano-18,20-(metheno)dipyrido[3,4-b:4′,3′- f][1,5,13]oxadiazacyclohexadecin-17(19H)-one |
| 125 | 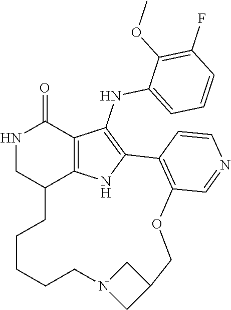 [SMILES: COc1c(F)cccc1Nc1c2[nH]c3c1C(=O)NCC3CCCCCN1CC(COc3cnccc3-2)C1] | 21-(3-fluoro-2-methoxyanilino)- 7,8,10,11,12,13,14,14a,15,16-decahydro-6H-7,9- methano-18,20-(metheno)dipyrido[3,4-b:4′,3′- f][1,5,13]oxadiazacyclohexadecin-17(19H)-one |
| 126 | 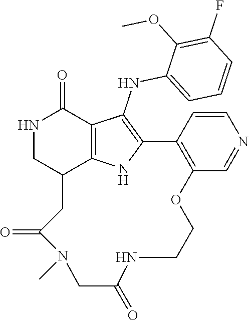 [SMILES: COc1c(F)cccc1Nc1c2[nH]c3c1C(=O)NCC3CC(=O)N(C)CC(=O)NCCOc1cnccc1-2] | 20-(3-fluoro-2-methoxyanilino)-11-methyl- 7,8,10,11,13,13a,14,15-octahydro-6H- 17,19-(metheno)dipyrido[3,4-j:4′,3′- n][1,4,7,12]oxatriazacyclopentadecine- 9,12,16(18H)-trione |
| 127 | 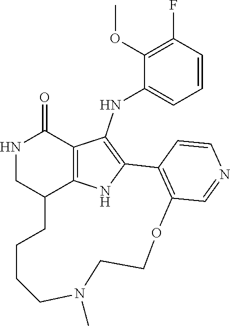 [SMILES: COc1c(F)cccc1Nc1c2[nH]c3c1C(=O)NCC3CCCCN(C)CCOc1cnccc1-2] | 19-(3-fluoro-2-methoxyanilino)-8-methyl- 6,7,8,9,10,11,12,12a,13,14-decahydro- 16,18-(metheno)dipyrido[3,4-i:4′,3′- m][1,4,11]oxadiazacyclotetradecin- 15(17H)-one |
| 128 | 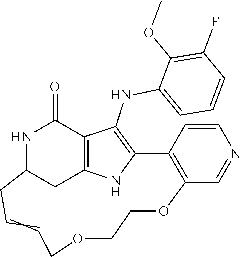 [SMILES: COc1c(F)cccc1Nc1c2[nH]c3c1C(=O)NC(CC=CCOCCOc1cnccc1-2)C3] | 20-(3-fluoro-2-methoxyanilino)- 2,3,5,8,9,10-hexahydro-9,13-methano- 12,15-(metheno)pyrido[3,4- e][1,4,8,12]dioxadiazacycloheptadecin- 11(14H)-one |
| 129 | 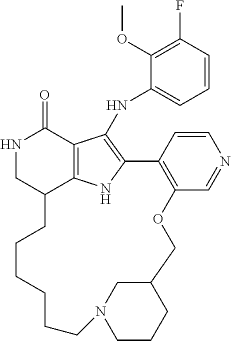 [SMILES: COc1c(F)cccc1Nc1c2[nH]c3c1C(=O)NCC3CCCCCCN1CCCC(COc3cnccc3-2)C1] | 24-(3-fluoro-2-methoxyanilino)- 7,8,9,10,13,14,15,16,17,17a,18,19-dodecahydro- 6H,12H,7,11-methano-21,23- (metheno)dipyrido[3,4-b:4′,3′- f][1,5,14]oxadiazacyclononadecin-20(22H)-one |
| 130 | 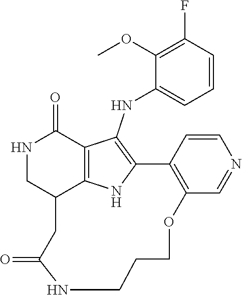 [SMILES: COc1c(F)cccc1Nc1c2[nH]c3c1C(=O)NCC3CC(=O)NCCCOc1cnccc1-2] | 18-(3-fluoro-2-methoxyanilino)- 8,9,11,11a,12,13-hexahydro-6H-15,17- (metheno)dipyrido[3,4-b:4′,3′- f][1,5,10]oxadiazacyclotridecine- 10,14(7H,16H)-dione |
| 131 | 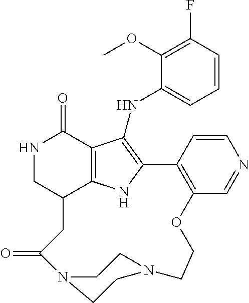 [SMILES: COc1c(F)cccc1Nc1c2[nH]c3c1C(=O)NCC3CC(=O)N1CCN(CCOc3cnccc3-2)CC1] | 20-(3-fluoro-2-methoxyanilino)- 6,7,9,10,13,13a,14,15-octahydro-12H-8,11- ethano-17,19-(metheno)dipyrido[3,4-j:4′,3′- n][1,4,7,12]oxatriazacyclopentadecine- 12,16(18H)-dione |
| 132 | 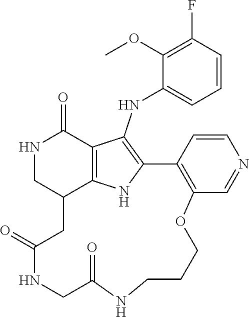 [SMILES: COc1c(F)cccc1Nc1c2[nH]c3c1C(=O)NCC3CC(=O)NCC(=O)NCCCOc1cnccc1-2] | 21-(3-fluoro-2-methoxyanilino)- 6,7,8,9,11,12,14,14a,15,16-decahydro- 18,20-(metheno)dipyrido[3,4-k:4′,3′- o][1,5,8,13]oxatriazacyclohexadecine- 10,13,17(19H)-trione |
| 133 | 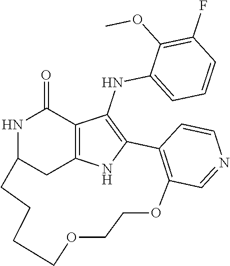 [SMILES: COc1c(F)cccc1Nc1c2[nH]c3c1C(=O)NC(CCCCOCCOc1cnccc1-2)C3] | 20-(3-fluoro-2-methoxyanilino)- 2,3,5,6,7,8,9,10-octahydro-9,13-methano- 12,15-(metheno)pyrido[3,4- e][1,4,8,12]dioxadiazacycloheptadecin- 11(14H)-one |
| 134 | 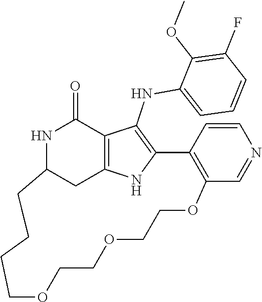 [SMILES: COc1c(F)cccc1Nc1c2[nH]c3c1C(=O)NC(CCCCOCCOCCOc1cnccc1-2)C3] | 23-(3-fluoro-2-methoxyanilino)- 2,3,5,6,8,9,10,11,12,13-decahydro-12,16- methano-15,18-(metheno)pyrido[3,4- h][1,4,7,11,15]trioxadiazacycloicosin- 14(17H)-one |
| 135 | 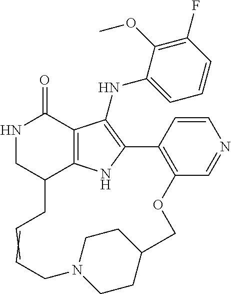 [SMILES: COc1c(F)cccc1Nc1c2[nH]c3c1C(=O)NCC3CC=CCN1CCC(CC1)COc1cnccc1-2] | 21-(3-fluoro-2-methoxyanilino)- 6,7,8,9,14,14a,15,16-octahydro-11H-7,10- ethano-18,20-(metheno)dipyrido[3,4-b:4′,3′- f][1,5,12]oxadiazacyclohexadecin-17(19H)-one |
| 136 | 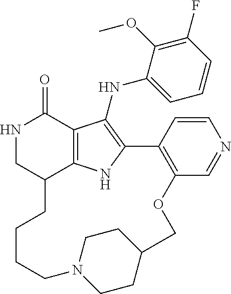 [SMILES: COc1c(F)cccc1Nc1c2[nH]c3c1C(=O)NCC3CCCCN1CCC(CC1)COc1cnccc1-2] | 21-(3-fluoro-2-methoxyanilino)- 6,7,8,9,12,13,14,14a,15,16-decahydro-11H-7,10- ethano-18,20-(metheno)dipyrido[3,4-b:4′,3′- f][1,5,12]oxadiazacyclohexadecin-17(19H)-one |
| 137 | 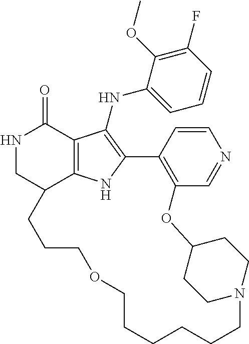 [SMILES: COc1c(F)cccc1Nc1c2[nH]c3c1C(=O)NCC3CCCOCCCCCCN1CCC(CC1)Oc1cnccc1-2] | 28-(3-fluoro-2-methoxyanilino)- 1,2,13,14,16,17,18,19,20,21,23,24,25,25a- tetradecahydro-12H-12,15-ethano-4,6- (metheno)dipyrido[3,4-b:4′,3′- f][1,11,5,18]dioxadiazacyclohenicosin-3(5H)- one |
| 138 | 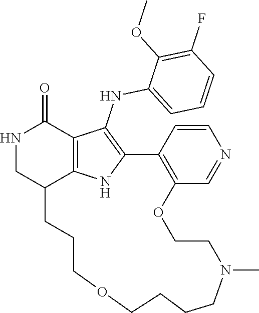 [SMILES: COc1c(F)cccc1Nc1c2[nH]c3c1C(=O)NCC3CCCOCCCCN(C)CCOc1cnccc1-2] | 23-(3-fluoro-2-methoxyanilino)-8-methyl- 7,8,9,10,11,12,14,15,16,16a,17,18- dodecahydro-6H-20,22- (metheno)dipyrido[3,4-m:4′,3′- q][1,9,4,15]dioxadiazacyclooctadecin- 19(21H)-one |
| 139 | 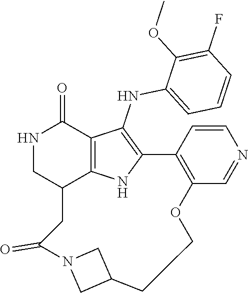 [SMILES: COc1c(F)cccc1Nc1c2[nH]c3c1C(=O)NCC3CC(=O)N1CC(CCOc3cnccc3-2)C1] | 19-(3-fluoro-2-methoxyanilino)- 6,7,8,9,12,12a,13,14-octahydro-11H-8,10- methano-16,18-(metheno)dipyrido[3,4-b:4′,3′- f][1,5,10]oxadiazacyclotetradecine-11,15(17H)- dione |
| 140 | 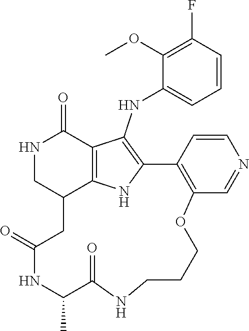 [SMILES: COc1c(F)cccc1Nc1c2[nH]c3c1C(=O)NCC3CC(=O)N[C@@H](C)C(=O)NCCCOc1cnccc1-2] | (11S)-21-(3-fluoro-2-methoxyanilino)-11- methyl-6,7,8,9,11,12,14,14a,15,16- decahydro-18,20-(metheno)dipyrido[3,4- k:4′,3′- o][1,5,8,13]oxatriazacyclohexadecine- 10,13,17(19H)-trione |
| 141 | 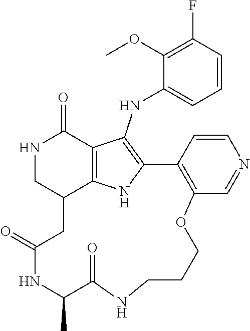 [SMILES: COc1c(F)cccc1Nc1c2[nH]c3c1C(=O)NCC3CC(=O)N[C@H](C)C(=O)NCCCOc1cnccc1-2] | (11R)-21-(3-fluoro-2-methoxyanilino)-11- methyl-6,7,8,9,11,12,14,14a,15,16- decahydro-18,20-(metheno)dipyrido[3,4- k:4′,3′- o][1,5,8,13]oxatriazacyclohexadecine- 10,13,17(19H)-trione |
| 142 | 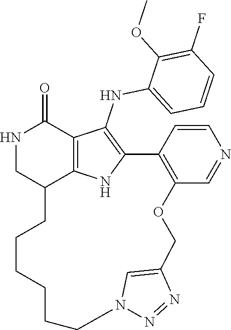 [SMILES: COc1c(F)cccc1Nc1c2[nH]c3c1C(=O)NCC3CCCCCCn1cc(nn1)COc1cnccc1-2] | 23-(3-fluoro-2-methoxyanilino)- 12,13,14,15,16,16a,17,18-octahydro-6H,11H- 10,7:20,22-di(metheno)dipyrido[3,4-m:4′,3′- q][1,4,5,6,15]oxatetraazacyclooctadecin- 19(21H)-one |
| 143 | 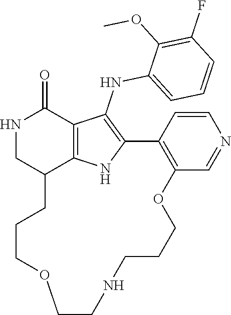 [SMILES: COc1c(F)cccc1Nc1c2[nH]c3c1C(=O)NCC3CCCOCCNCCCOc1cnccc1-2] | 22-(3-fluoro-2-methoxyanilino)- 6,7,8,9,10,11,13,14,15,15a,16,17- dodecahydro-19,21-(metheno)dipyrido[3,4- i:4′,3′- m][1,8,4,12]dioxadiazacycloheptadecin- 18(20H)-one |
| 144 | 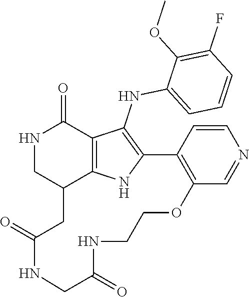 [SMILES: COc1c(F)cccc1Nc1c2[nH]c3c1C(=O)NCC3CC(=O)NCC(=O)NCCOc1cnccc1-2] | 20-(3-fluoro-2-methoxyanilino)- 7,8,10,11,13,13a,14,15-octahydro-6H- 17,19-(metheno)dipyrido[3,4-j:4′,3′- n][1,4,7,12]oxatriazacyclopentadecine- 9,12,16(18H)-trione |
| 145 | 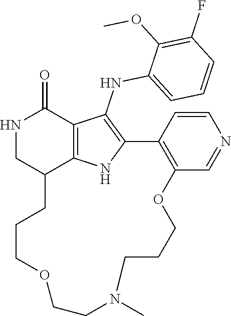 [SMILES: COc1c(F)cccc1Nc1c2[nH]c3c1C(=O)NCC3CCCOCCN(C)CCCOc1cnccc1-2] | 22-(3-fluoro-2-methoxyanilino)-9-methyl- 6,7,8,9,10,11,13,14,15,15a,16,17- dodecahydro-19,21-(metheno)dipyrido[3,4- i:4′,3′- m][1,8,4,12]dioxadiazacycloheptadecin- 18(20H)-one |
| 146 | 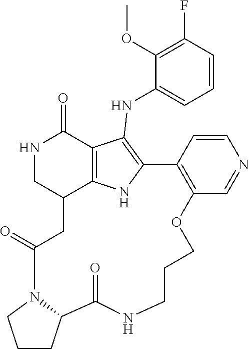 [SMILES: COc1c(F)cccc1Nc1c2[nH]c3c1C(=O)NCC3CC(=O)N1CCC[C@H]1C(=O)NCCCOc1cnccc1-2] | (16aS)-23-(3-fluoro-2-methoxyanilino)- 14,15,16a,17,18,19,22,22a-octahydro- 2H,12H-4,6-(metheno)dipyrido[3,4-k:4′,3′- o]pyrrolo[2,1- g][1,5,8,13]oxatriazacyclohexadecine- 3,16,21(1H,5H,13H)-trione |
| 147 | 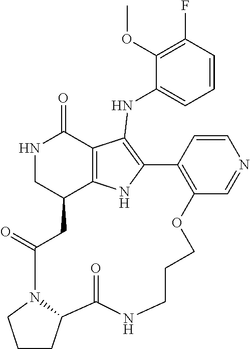 [SMILES: COc1c(F)cccc1Nc1c2[nH]c3c1C(=O)NC[C@@H]3CC(=O)N1CCC[C@H]1C(=O)NCCCOc1cnccc1-2] | (16aS,22aS)-23-(3-fluoro-2- methoxyanilino)- 14,15,16a,17,18,19,22,22a-octahydro- 2H,12H-4,6-(metheno)dipyrido[3,4-k:4′,3′- o]pyrrolo[2,1- g][1,5,8,13]oxatriazacyclohexadecine- 3,16,21(1H,5H,13H)-trione |
| 148 | 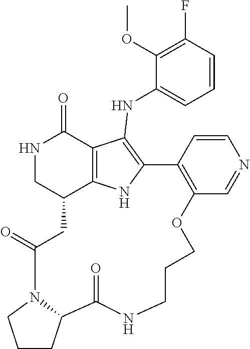 [SMILES: COc1c(F)cccc1Nc1c2[nH]c3c1C(=O)NC[C@H]3CC(=O)N1CCC[C@H]1C(=O)NCCCOc1cnccc1-2] | (16aS,22aR)-23-(3-fluoro-2- methoxyanilino)- 14,15,16a,17,18,19,22,22a-octahydro- 2H,12H-4,6-(metheno)dipyrido[3,4-k:4′,3′- o]pyrrolo[2,1- g][1,5,8,13]oxatriazacyclodecine- 3,16,21(1H,5H,13H)-trione |
| 149 | 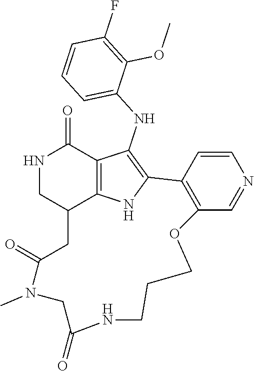 [SMILES: COc1c(F)cccc1Nc1c2[nH]c3c1C(=O)NCC3CC(=O)N(C)CC(=O)NCCCOc1cnccc1-2] | 21-(3-fluoro-2-methoxyanilino)-12-methyl- 6,7,8,9,11,12,14,14a,15,16-decahydro- 18,20-(metheno)dipyrido[3,4-k:4′,3′- o][1,5,8,13]oxatriazacyclohexadecine- 10,13,17(19H)-trione |
| 150 | 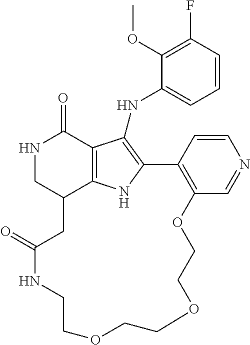 [SMILES: COc1c(F)cccc1Nc1c2[nH]c3c1C(=O)NCC3CC(=O)NCCOCCOCCOc1cnccc1-2] | 23-(3-fluoro-2-methoxyanilino)- 6,7,9,10,13,14,16,16a,17,18-decahydro- 20,22-(metheno)dipyrido[3,4-m:4′,3′- q][1,4,7,10,15]trioxadiazacyclooctadecine- 15,19(12H,21H)-dione |
| 151 | 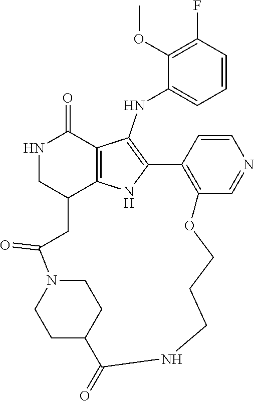 [SMILES: COc1c(F)cccc1Nc1c2[nH]c3c1C(=O)NCC3CC(=O)N1CCC(CC1)C(=O)NCCCOc1cnccc1-2] | 23-(3-fluoro-2-methoxyanilino)- 6,7,8,9,12,13,16,16a,17,18-decahydro-15H- 11,14-ethano-20,22-(metheno)dipyrido[3,4- b:4′,3′- f][1,5,10,15]oxatriazacyclooctadecine- 10,15,19(11H,21H)-trione |
| 152 | 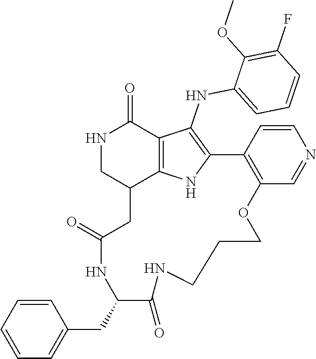 [SMILES: COc1c(F)cccc1Nc1c2[nH]c3c1C(=O)NCC3CC(=O)N[C@@H](Cc1ccccc1)C(=O)NCCCOc1cnccc1-2] | (11S)-11-benzyl-21-(3-fluoro-2- methoxyanilino)- 6,7,8,9,11,12,14,14a,15,16-decahydro- 18,20-(metheno)dipyrido[3,4-k:4′,3′- o][1,5,8,13]oxatriazacyclohexadecine- 10,13,17(19H)-trione |
| 153 | 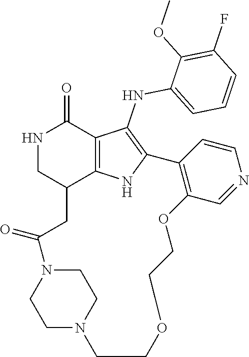 [SMILES: COc1c(F)cccc1Nc1c2[nH]c3c1C(=O)NCC3CC(=O)N1CCN(CCOCCOc3cnccc3-2)CC1] | 23-(3-fluoro-2-methoxyanilino)- 6,7,9,10,12,13,16,16a,17,18-decahydro- 15H-11,14-ethano-20,22- (metheno)dipyrido[3,4-m:4′,3′- q][1,4,7,10,15]dioxatriazacyclooctadecine- 15,19(21H)-dione trifluoroacetic acid (1/1) |
| 154 | 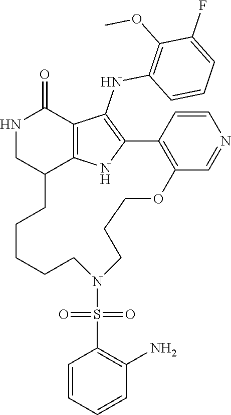 [SMILES: COc1c(F)cccc1Nc1c2[nH]c3c1C(=O)NCC3CCCCCN(S(=O)(=O)c1ccccc1N)CCCOc1cnccc1-2] | 9-(2-aminobenzene-1-sulfonyl)-21-(3- fluoro-2-methoxyanilino)- 6,7,8,9,10,11,12,13,14,14a,15,16- dodecahydro-18,20-(metheno)dipyrido[3,4- b:4′,3′-f][1,5,13]oxadiazacyclohexadecin- 17(19H)-one |
| 155 | 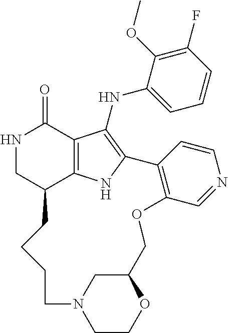 [SMILES: COc1c(F)cccc1Nc1c2[nH]c3c1C(=O)NC[C@@H]3CCCCN1CCO[C@H](COc3cnccc3-2)C1] | (7S,15aS)-22-(3-fluoro-2-methoxyanilino)- 6,7,9,10,13,14,15,15a,16,17-decahydro-12H- 7,11-methano-19,21-(metheno)dipyrido[3,4- l:4′,3′-p][1,4,7,14]dioxadiazacycloheptadecin- 18(20H)-one |
| 156 | 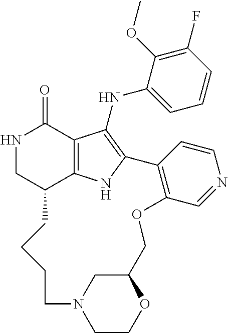 [SMILES: COc1c(F)cccc1Nc1c2[nH]c3c1C(=O)NC[C@H]3CCCCN1CCO[C@H](COc3cnccc3-2)C1] | (7S,15aR)-22-(3-fluoro-2-methoxyanilino)- 6,7,9,10,13,14,15,15a,16,17-decahydro-12H- 7,11-methano-19,21-(metheno)dipyrido[3,4- l:4′,3′-p][1,4,7,14]dioxadiazacycloheptadecin- 18(20H)-one |
| 157 | 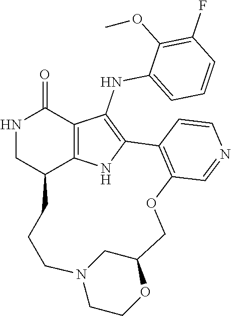 [SMILES: COc1c(F)cccc1Nc1c2[nH]c3c1C(=O)NC[C@@H]3CCCN1CCO[C@H](COc3cnccc3-2)C1] | (7S,14aS)-21-(3-fluoro-2-methoxyanilino)- 6,7,9,10,12,13,14,14a,15,16-decahydro-7,11- methano-18,20-(metheno)dipyrido[3,4-k:4′,3′- o][1,4,7,13]dioxadiazacyclohexadecin-17(19H)- one |
| 158 | 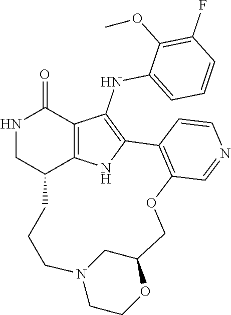 [SMILES: COc1c(F)cccc1Nc1c2[nH]c3c1C(=O)NC[C@H]3CCCN1CCO[C@H](COc3cnccc3-2)C1] | (7S,14aR)-21-(3-fluoro-2-methoxyanilino)- 6,7,9,10,12,13,14,14a,15,16-decahydro-7,11- methano-18,20-(metheno)dipyrido[3,4-k:4′,3′- o][1,4,7,13]dioxadiazacyclohexadecin-17(19H)- one |
| 159 | 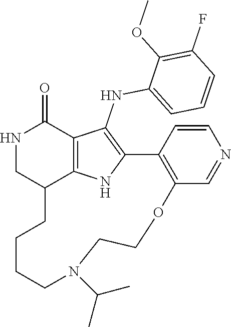 [SMILES: COc1c(F)cccc1Nc1c2[nH]c3c1C(=O)NCC3CCCCN(C(C)C)CCOc1cnccc1-2] | 19-(3-fluoro-2-methoxyanilino)-8-(propan- 2-yl)-6,7,8,9,10,11,12,12a,13,14-decahydro- 16,18-(metheno)dipyrido[3,4-i:4′,3′- m][1,4,11]oxadiazacyclotetradecin- 15(17H)-one |
| 160 | 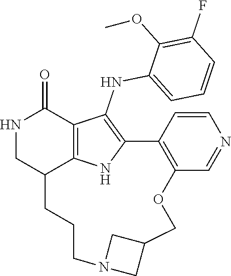 [SMILES: COc1c(F)cccc1Nc1c2[nH]c3c1C(=O)NCC3CCCN1CC(COc3cnccc3-2)C1] | 19-(3-fluoro-2-methoxyanilino)- 7,8,10,11,12,12a,13,14-octahydro-6H-7,9- methano-16,18-(metheno)dipyrido[3,4- b:4′,3′-f][1,5,11]oxadiazacyclotetradecin- 15(17H)-one |
| 161 | 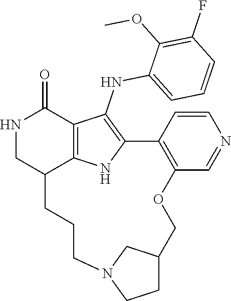 [SMILES: COc1c(F)cccc1Nc1c2[nH]c3c1C(=O)NCC3CCCN1CCC(COc3cnccc3-2)C1] | 20-(3-fluoro-2-methoxyanilino)- 6,7,8,9,11,12,13,13a,14,15-decahydro-7,10- methano-17,19-(metheno)dipyrido[3,4-b:4′,3′- f][1,5,11]oxadiazacyclopentadecin-16(18H)-one |
| 162 | 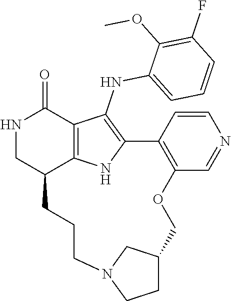 [SMILES: COc1c(F)cccc1Nc1c2[nH]c3c1C(=O)NC[C@@H]3CCCN1CC[C@@H](COc3cnccc3-2)C1] | (7R,13aS)-20-(3-fluoro-2-methoxyanilino)- 6,7,8,9,11,12,13,13a,14,15-decahydro-7,10- methano-17,19-(metheno)dipyrido[3,4-b:4′,3′- f][1,5,11]oxadiazacyclopentadecin-16(18H)-one |
| 163 | 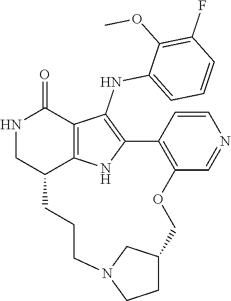 [SMILES: COc1c(F)cccc1Nc1c2[nH]c3c1C(=O)NC[C@H]3CCCN1CC[C@@H](COc3cnccc3-2)C1] | (7R,13aR)-20-(3-fluoro-2-methoxyanilino)- 6,7,8,9,11,12,13,13a,14,15-decahydro-7,10- methano-17,19-(metheno)dipyrido[3,4-b:4′,3′- f][1,5,11]oxadiazacyclopentadecin-16(18H)-one |
| 164 | 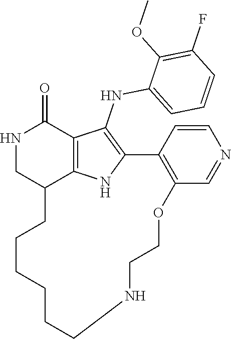 [SMILES: COc1c(F)cccc1Nc1c2[nH]c3c1C(=O)NCC3CCCCCCNCCOc1cnccc1-2] | 21-(3-fluoro-2-methoxyanilino)- 6,7,8,9,10,11,12,13,14,14a,15,16- dodecahydro-18,20-(metheno)dipyrido[3,4- k:4′,3′-o][1,4,13]oxadiazacyclohexadecin- 17(19H)-one |
| 165 | 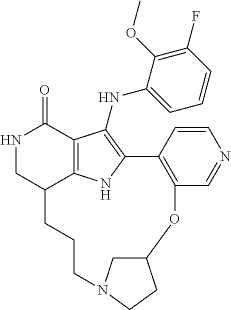 [SMILES: COc1c(F)cccc1Nc1c2[nH]c3c1C(=O)NCC3CCCN1CCC(C1)Oc1cnccc1-2] | 19-(3-fluoro-2-methoxyanilino)- 7,8,10,11,12,12a,13,14-octahydro-6H-6,9- methano-16,18-(metheno)dipyrido[3,4-b:4′,3′- f][1,5,11]oxadiazacyclotetradecin-15(17H)-one |
| 166 | 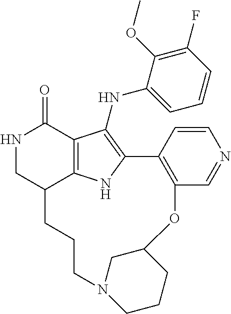 [SMILES: COc1c(F)cccc1Nc1c2[nH]c3c1C(=O)NCC3CCCN1CCCC(C1)Oc1cnccc1-2] | 20-(3-fluoro-2-methoxyanilino)- 6,7,8,9,11,12,13,13a,14,15-decahydro-6,10- methano-17,19-(metheno)dipyrido[3,4-b:4′,3′- f][1,5,11]oxadiazacyclopentadecin-16(18H)-one |
| 167 | 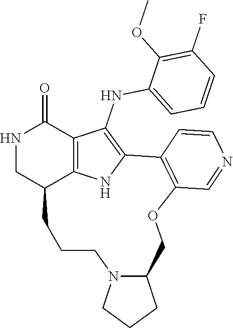 [SMILES: COc1c(F)cccc1Nc1c2[nH]c3c1C(=O)NC[C@@H]3CCCN1CCC[C@@H]1COc1cnccc1-2] | (12aR,19aS)-20-(3-fluoro-2-methoxyanilino)- 1,2,12a,13,14,15,17,18,19,19a-decahydro-12H- 4,6-(metheno)dipyrido[3,4-h:4′,3′-l]pyrrolo[2,1- c][1,4,10]oxadiazacyclotridecin-3(5H)-one |
| 168 | 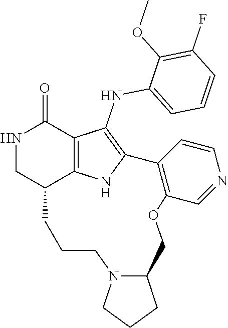 [SMILES: COc1c(F)cccc1Nc1c2[nH]c3c1C(=O)NC[C@H]3CCCN1CCC[C@@H]1COc1cnccc1-2] | (12aR,19aR)-20-(3-fluoro-2-methoxyanilino)- 1,2,12a,13,14,15,17,18,19,19a-decahydro-12H- 4,6-(metheno)dipyrido[3,4-h:4′,3′-l]pyrrolo[2,1- c][1,4,10]oxadiazacyclotridecin-3(5H)-one |
| 169 | 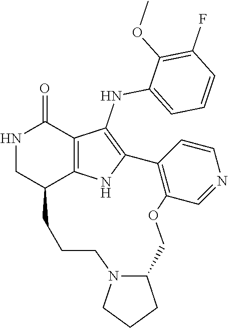 [SMILES: COc1c(F)cccc1Nc1c2[nH]c3c1C(=O)NC[C@@H]3CCCN1CCC[C@H]1COc1cnccc1-2] | (12aS,19aS)-20-(3-fluoro-2-methoxyanilino)- 1,2,12a,13,14,15,17,18,19,19a-decahydro-12H- 4,6-(metheno)dipyrido[3,4-h:4′,3′-l]pyrrolo[2,1- c][1,4,10]oxadiazacyclotridecin-3(5H)-one |
| 170 | 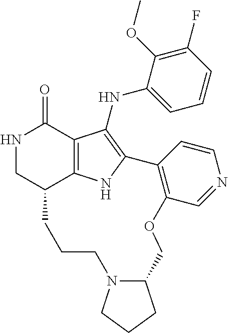 [SMILES: COc1c(F)cccc1Nc1c2[nH]c3c1C(=O)NC[C@H]3CCCN1CCC[C@H]1COc1cnccc1-2] | (12aS,19aR)-20-(3-fluoro-2-methoxyanilino)- 1,2,12a,13,14,15,17,18,19,19a-decahydro-12H- 4,6-(metheno)dipyrido[3,4-h:4′,3′-l]pyrrolo[2,1- c][1,4,10]oxadiazacyclotridecin-3(5H)-one |

or a pharmaceutically acceptable salt, stereoisomer, solvate, or tautomer thereof.

19. The compound of claim 16, wherein the compound is selected from:

| # | Structure | IUPAC name |
|---|---|---|
| 2 | 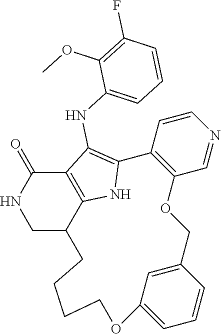 [SMILES: COc1c(F)cccc1Nc1c2[nH]c3c1C(=O)NCC3CCCCOc1cccc(c1)COc1cnccc1-2] | 28-[(3-fluoro-2-methoxyphenyl)amino]-3,22-dioxa-6,11,15- triazapentacyclo[21.3.1.1^{10,13}.0^{4,9}.0^{12,17}]octacosa- 1(27),4,6,8,10(28),12,23,25-octaen-14-one |
| 3 | 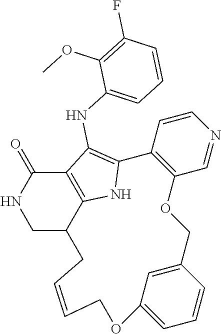 [SMILES: COc1c(F)cccc1Nc1c2[nH]c3c1C(=O)NCC3C/C=C\COc1cccc(c1)COc1cnccc1-2] | (19Z)-28-[(3-fluoro-2-methoxyphenyl)amino]-3,22-dioxa-6,11,15- triazapentacyclo[21.3.1.1^{10,13}.0^{4,9}.0^{12,17}]octacosa- 1(27),4,6,8,10(28),12,19,23,25-nonaen-14-one |
| 4 | 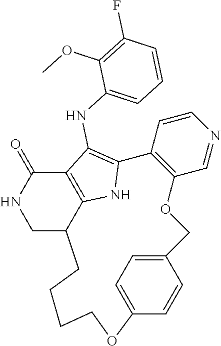 [SMILES: COc1c(F)cccc1Nc1c2[nH]c3c1C(=O)NCC3CCCCOc1ccc(cc1)COc1cnccc1-2] | 28-[(3-fluoro-2-methoxyphenyl)amino]-3,22-dioxa-6,11,15- triazapentacyclo[21.2.2.1^{10,13}0.^{4,9}.0^{12,17}]octacosa- 1(25), 4,6,8,10(28),12,23,26-octaen-14-one |
| 5 | 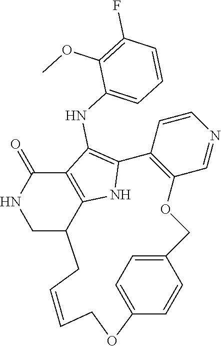 [SMILES: COc1c(F)cccc1Nc1c2[nH]c3c1C(=O)NCC3C/C=C\COc1ccc(cc1)COc1cnccc1-2] | (19Z)-28-[(3-fluoro-2-methoxyphenyl)amino]-3,22-dioxa-6,11,15- triazapentacyclo[21.2.2.1^{10,13}.0^{4,9}.0^{12,17}]octacosa- 1(25),4,6,8,10(28),12,19,23,26-nonaen-14-one |
| 6 | 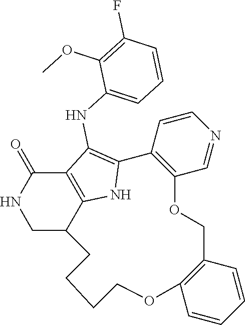 [SMILES: COc1c(F)cccc1Nc1c2[nH]c3c1C(=O)NCC3CCCCOc1ccccc1COc1cnccc1-2] | 28-[(3-fluoro-2-methoxyphenyl)amino]-8,16-dioxa-5,23,27- triazapentacyclo[23.2.1.0^{2,7}.0^{10,15}.0^{21,26}]octacosa- 1(28),2,4,6,10,12,14,25-octaen-24-one |
| 7 | 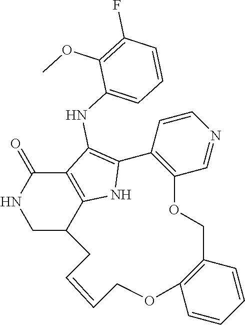 [SMILES: COc1c(F)cccc1Nc1c2[nH]c3c1C(=O)NCC3C/C=C\COc1ccccc1COc1cnccc1-2] | (18Z)-28-[(3-fluoro-2-methoxyphenyl)amino]-8,16-dioxa-5,23,27- triazapentacyclo[23.2.1.0^{2,7}.0^{10,15}.0^{21,26}]octacosa- 1(28),2,4,6,10,12,14,18,25-nonaen-24-one |
| 11 | 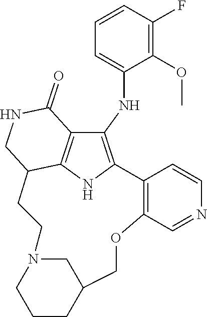 [SMILES: COc1c(F)cccc1Nc1c2[nH]c3c1C(=O)NCC3CCN1CCCC(COc3cnccc3-2)C1] | 25-[(3-fluoro-2-methoxyphenyl)amino]-18-oxa-1,6,10,15- tetraazapentacyclo[18.3.1.1^{8,11}.0^{4,9}.0^{12,17}]- pentacosa-8,11(25),12,14,16-pentaen-7-one |
| 12 | 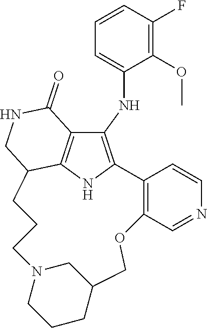 [SMILES: COc1c(F)cccc1Nc1c2[nH]c3c1C(=O)NCC3CCCN1CCCC(COc3cnccc3-2)C1] | 26-[(3-fluoro-2-methoxyphenyl)amino]-3-oxa-6,11,15,21- tetraazapentacyclo[19.3.1.1^{10,13}.0^{4,9}.0^{12,17}]- hexacosa-4,6,8,10(26),12-pentaen-14-one |
| 14 | 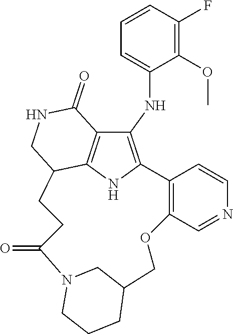 [SMILES: COc1c(F)cccc1Nc1c2[nH]c3c1C(=O)NCC3CCC(=O)N1CCCC(COc3cnccc3-2)C1] | 26-[(3-fluoro-2-methoxyphenyl)amino]-3-oxa-6,11,15,21- tetraazapentacyclo[19.3.1.1^{10,13}.0^{4,9}.0^{12,17}]- hexacosa-4,6,8,10(26),12-pentaene-14,20-dione |
| 29 | 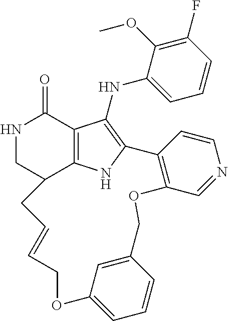 [SMILES: COc1c(F)cccc1Nc1c2[nH]c3c1C(=O)NCC3C/C=C/COc1cccc(c1)COc1cnccc1-2] | (19E)-28-[(3-fluoro-2-methoxyphenyl)amino]-3,22-dioxa-6,11,15- triazapentacyclo[21.3.1.1^{10,13}.0^{4,9}.0^{12,17}]octacosa- 1(27),4,6,8,10(28),12,19,23,25-nonaen-14-one |
| 30 | 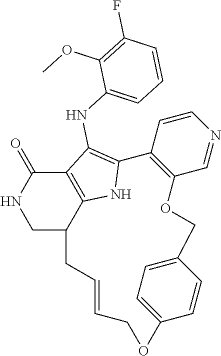 [SMILES: COc1c(F)cccc1Nc1c2[nH]c3c1C(=O)NCC3C/C=C/COc1ccc(cc1)COc1cnccc1-2] | (19E)-28-[(3-fluoro-2-methoxyphenyl)amino]-3,22-dioxa-6,11,15- triazapentacyclo[21.2.2.1^{10,13}.0^{4,9}.0^{12,17}]octacosa- 1(25),4,6,8,10(28),12,19,23,26-nonaen-14-one |
| 31 | 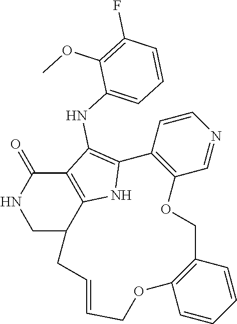 [SMILES: COc1c(F)cccc1Nc1c2[nH]c3c1C(=O)NCC3C/C=C/COc1ccccc1COc1cnccc1-2] | (18E)-28-[(3-fluoro-2-methoxyphenyl)amino]-8,16-dioxa-5,23,27- triazapentacyclo[23.2.1.0^{2,7}.0^{10,15}.0^{21,26}]octacosa- 1(28),2,4,6,10,12,14,18,25-nonaen-24-one |
| 42 | 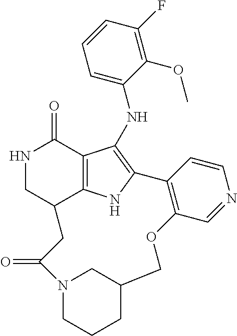 [SMILES: COc1c(F)cccc1Nc1c2[nH]c3c1C(=O)NCC3CC(=O)N1CCCC(COc3cnccc3-2)C1] | 25-[(3-fluoro-2-methoxyphenyl)amino]-18-oxa-1,6,10,15- tetraazapentacyclo[18.3.1.1^{8,11}.0^{4,9}.0^{12,17}]- pentacosa-8,11(25),12,14,16-pentaene-2,7-dione |
| 43 | 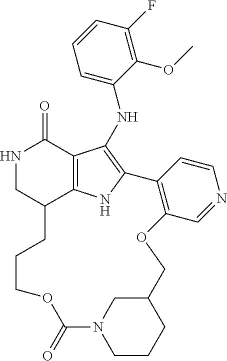 [SMILES: COc1c(F)cccc1Nc1c2[nH]c3c1C(=O)NCC3CCCOC(=O)N1CCCC(COc3cnccc3-2)C1] | 28-[(3-fluoro-2-methoxyphenyl)amino]-3,21-dioxa-6,11,15,23- tetraazapentacyclo[21.3.1.1^{10,13}.0^{4,9}.0^{12,17}]- octacosa-4,6,8,10(28),12-pentaene-14,22-dione |
| 44 | 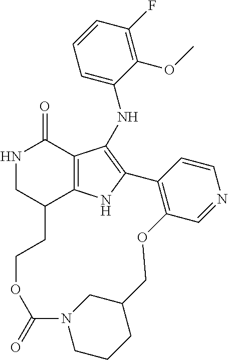 [SMILES: COc1c(F)cccc1Nc1c2[nH]c3c1C(=O)NCC3CCOC(=O)N1CCCC(COc3cnccc3-2)C1] | 27-[(3-fluoro-2-methoxyphenyl)amino]-3,20-dioxa-6,11,15,22- tetraazapentacyclo[20.3.1.1^{10,13}.0^{4,9}.0^{12,17}]- heptacosa-4,6,8,10(27),12-pentaene-14,21-dione |
| 70 | 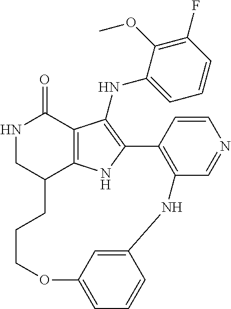 [SMILES: COc1c(F)cccc1Nc1c2[nH]c3c1C(=O)NCC3CCCOc1cccc(c1)Nc1cnccc1-2] | 26-[(3-fluoro-2-methoxyphenyl)amino]-20-oxa-2,5,10,14- tetraazapentacyclo[19.3.1.1^{9,12}.0^{3,8}.0^{11,16}]- hexacosa-1(25),3,5,7,9(26),11,21,23-octaen-13-one |
| 71 | 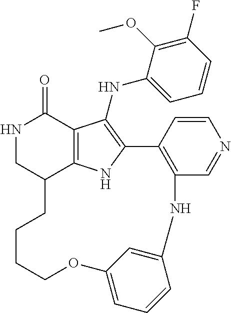 [SMILES: COc1c(F)cccc1Nc1c2[nH]c3c1C(=O)NCC3CCCCOc1cccc(c1)Nc1cnccc1-2] | 27-[(3-fluoro-2-methoxyphenyl)amino]-21-oxa-2,5,10,14- tetraazapentacyclo[20.3.1.1^{9,12}.0^{3,8}.0^{11,16}]- heptacosa-1(26),3,5,7,9(27),11,22,24-octaen-13- one |
| 80 | 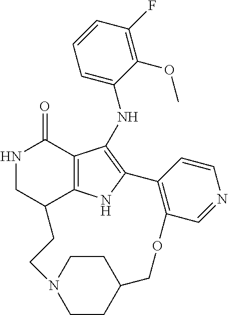 [SMILES: COc1c(F)cccc1Nc1c2[nH]c3c1C(=O)NCC3CCN1CCC(CC1)COc1cnccc1-2] | 25-[(3-fluoro-2-methoxyphenyl)amino]-18-oxa-1,6,10,15- tetraazapentacyclo[18.2.2.1^{8,11}.0^{4,9}.0^{12,17}]pentacosa- 8,11(25),12,14,16-pentaen-7-one |
| 81 | 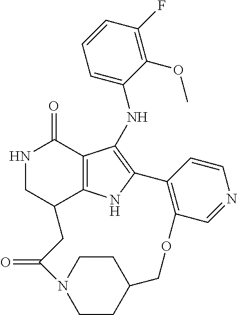 [SMILES: COc1c(F)cccc1Nc1c2[nH]c3c1C(=O)NCC3CC(=O)N1CCC(CC1)COc1cnccc1-2] | 25-[(3-fluoro-2-methoxyphenyl)amino]- 18-oxa-1,6,10,15-tetraazapentacyclo [18.2.2.1^{8,11}.0^{4,9}.0^{12,17}]pentacosa- 8,11(25),12,14,16-pentaene-2,7-dione |
| 82 | 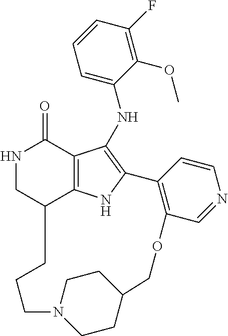 [SMILES: COc1c(F)cccc1Nc1c2[nH]c3c1C(=O)NCC3CCCN1CCC(CC1)COc1cnccc1-2] | 26-[(3-fluoro-2-methoxyphenyl)amino]-3-oxa- 6,11,15,21-tetraazapentacyclo [19.2.2.1^{10,13}.0^{4,9}.0^{12,17}]- hexacosa-4,6,8,10(26),12-pentaen-14-one |
| 83 | 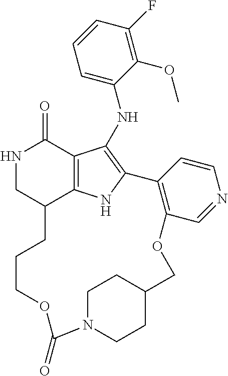 [SMILES: COc1c(F)cccc1Nc1c2[nH]c3c1C(=O)NCC3CCCOC(=O)N1CCC(CC1)COc1cnccc1-2] | 28-[(3-fluoro-2-methoxyphenyl)amino]-3,21- dioxa-6,11,15,23-tetraazapentacyclo [21.2.2.1^{10,13}.0^{4,9}.0^{12,17}]- octacosa-4,6,8,10(28),12-pentaene-14,22-dione |
| 84 | 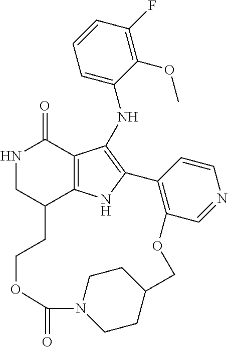 [SMILES: COc1c(F)cccc1Nc1c2[nH]c3c1C(=O)NCC3CCOC(=O)N1CCC(CC1)COc1cnccc1-2] | 27-[(3-fluoro-2-methoxyphenyl)amino]-3,20- dioxa-6,11,15,22- tetraazapentacyclo[20.2.2.1^{10,13}.0^{4,9}.0^{12,17}]- heptacosa-4,6,8,10(27),12-pentaene-14,21-dione |
| 91 | 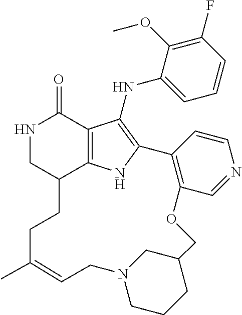 [SMILES: COc1c(F)cccc1Nc1c2[nH]c3c1C(=O)NCC3CC/C(C)=C\CN1CCCC(COc3cnccc3-2)C1] | (20Z)-28-[(3-fluoro-2-methoxyphenyl)amino]- 20-methyl-3-oxa-6,11,15,23- tetraazapentacyclo[21.3.1.1^{10,13}.0^{4,9}.0^{12,17}]- octacosa-4,6,8,10(28),12,20-hexaen-14-one |
| 92 | 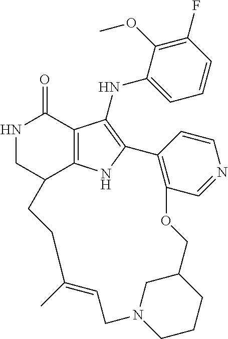 [SMILES: COc1c(F)cccc1Nc1c2[nH]c3c1C(=O)NCC3CC/C(C)=C/CN1CCCC(COc3cnccc3-2)C1] | (20E)-28-[(3-fluoro-2-methoxyphenyl)amino]- 20-methyl-3-oxa-6,11,15,23- tetraazapentacyclo[21.3.1.1^{10,13}.0^{4,9}.0^{12,17}]- octacosa-4,6,8,10(28),12,20-hexaen-14-one |
| 93 | 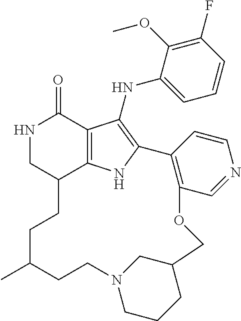 [SMILES: COc1c(F)cccc1Nc1c2[nH]c3c1C(=O)NCC3CCC(C)CCN1CCCC(COc3cnccc3-2)C1] | 28-[(3-fluoro-2-methoxyphenyl)amino]-20- methyl-3-oxa-6,11,15,23- tetraazapentacyclo[21.3.1.1^{10,13}.0^{4,9}.0^{12,17}]- octacosa-4,6,8,10(28),12-pentaen-14-one |
| 94 | 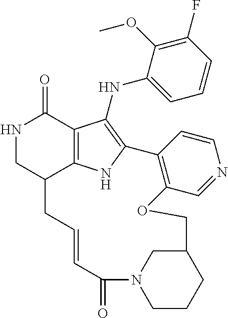 [SMILES: COc1c(F)cccc1Nc1c2[nH]c3c1C(=O)NCC3C/C=C/C(=O)N1CCCC(COc3cnccc3-2)C1] | 27-[(3-fluoro-2-methoxyphenyl)amino]-3-oxa- 6,11,15,22- tetraazapentacyclo[20.3.1.1^{10,13}.0^{4,9}.0^{12,17}]- heptacosa-4,6,8,10(27),12,19-hexaene-14,21- dione |
| 95 | 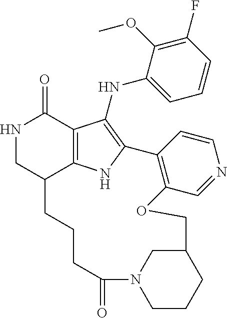 [SMILES: COc1c(F)cccc1Nc1c2[nH]c3c1C(=O)NCC3CCCC(=O)N1CCCC(COc3cnccc3-2)C1] | 27-[(3-fluoro-2-methoxyphenyl)amino]-3-oxa- 6,11,15,22- tetraazapentacyclo[20.3.1.1^{10,13}.0^{4,9}.0^{12,17}]- heptacosa-4,6,8,10(27),12-pentaene-14,21-dione |
| 96 | 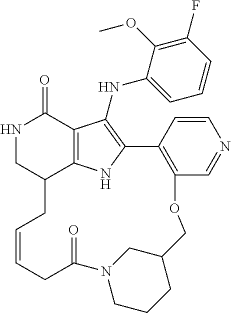 [SMILES: COc1c(F)cccc1Nc1c2[nH]c3c1C(=O)NCC3C/C=C\CC(=O)N1CCCC(COc3cnccc3-2)C1] | (19Z)-28-[(3-fluoro-2-methoxyphenyl)amino]-3- oxa-6,11,15,23- tetraazapentacyclo[21.3.1.1^{10,13}.0^{4,9}.0^{12,17}]- octacosa-4,6,8,10(28),12,19-hexaene-14,22- dione |
| 97 | 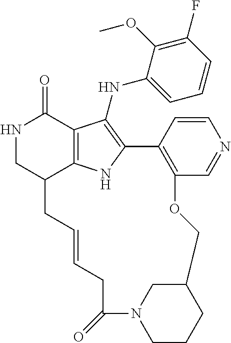 [SMILES: COc1c(F)cccc1Nc1c2[nH]c3c1C(=O)NCC3C/C=C/CC(=O)N1CCCC(COc3cnccc3-2)C1] | (19E)-28-[(3-fluoro-2-methoxyphenyl)amino]-3- oxa-6,11,15,23- tetraazapentacyclo[21.3.1.1^{10,13}.0^{4,9}.0^{12,17}]- octacosa-4,6,8,10(28),12,19-hexaene-14,22- dione |
| 98 | 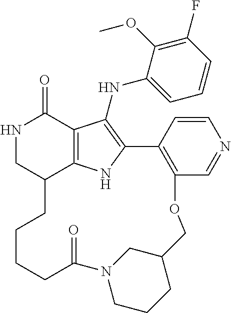 [SMILES: COc1c(F)cccc1Nc1c2[nH]c3c1C(=O)NCC3CCCCC(=O)N1CCCC(COc3cnccc3-2)C1] | 28-[(3-fluoro-2-methoxyphenyl)amino]-3-oxa- 6,11,15,23- tetraazapentacyclo[21.3.1.1^{10,13}.0^{4,9}.0^{12,17}]- octacosa-4,6,8,10(28),12-pentaene-14,22-dione |
| 102 | 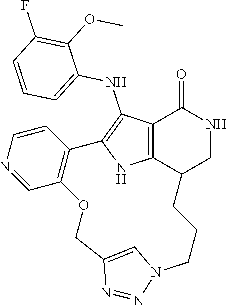 [SMILES: COc1c(F)cccc1Nc1c2[nH]c3c1C(=O)NCC3CCCn1cc(nn1)COc1cnccc1-2] | 24-[(3-fluoro-2-methoxyphenyl)amino]-8-oxa- 5,11,12,13,19,23- hexaazapentacyclo[19.2.1.1^{10,13}.0^{2,7}.0^{17,22}]- pentacosa-1(24),2,4,6,10(25),11,21-heptaen-20- one |
| 103 | 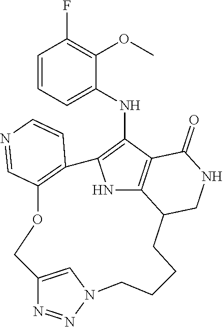 [SMILES: COc1c(F)cccc1Nc1c2[nH]c3c1C(=O)NCC3CCCCn1cc(nn1)COc1cnccc1-2] | 25-[(3-fluoro-2-methoxyphenyl)amino]-8-oxa- 5,11,12,13,20,24- hexaazapentacyclo[20.2.1.1^{10,13}.0^{2,7}.0^{18,23}]- hexacosa-1(25),2,4,6,10(26),11,22-heptaen-21- one |
| 107 | 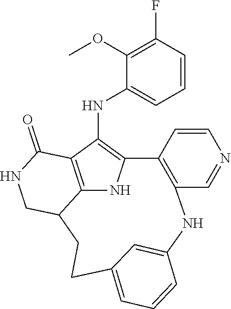 [SMILES: COc1c(F)cccc1Nc1c2[nH]c3c1C(=O)NCC3CCc1cccc(c1)Nc1cnccc1-2] | 24-[(3-fluoro-2-methoxyphenyl)amino]- 2,5,10,14- tetraazapentacyclo[17.3.1.1^{9,12}.0^{3,8}.0^{11,16}]- tetracosa-1(23),3,5,7,9(24),11,19,21-octaen-13- one |
| 116 | 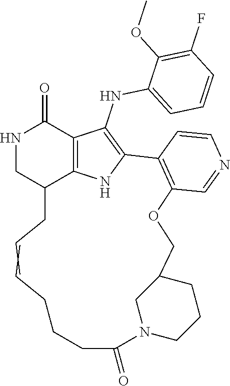 [SMILES: COc1c(F)cccc1Nc1c2[nH]c3c1C(=O)NCC3CC=CCCCC(=O)N1CCCC(COc3cnccc3-2)C1] | 25-[(3-fluoro-2-methoxyphenyl)amino]- 7,8,9,10,14,15,18,18a,19,20-decahydro-6H-7,11- methano-2,24-methenodipyrido[3,4-b:4′,3′- f][1,5,15]oxadiazacycloicosine-12,21(13H,23H)- dione |
| 117 | 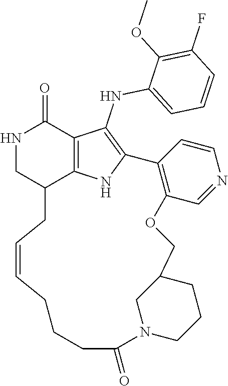 [SMILES: COc1c(F)cccc1Nc1c2[nH]c3c1C(=O)NCC3C/C=C\CCCC(=O)N1CCCC(COc3cnccc3-2)C1] | Z-25-[(3-fluoro-2-methoxyphenyl)amino]- 7,8,9,10,14,15,18,18a,19,20-decahydro-6H-7,11- methano-22,24-methenopyrido[3,4-b:4′,3′- f][1,5,15]oxadiazacycloicosine-12,21(13H,23H)- dione |
| 118 | 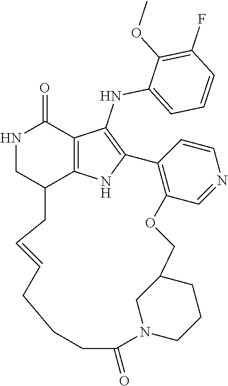 [SMILES: COc1c(F)cccc1Nc1c2[nH]c3c1C(=O)NCC3C/C=C/CCCC(=O)N1CCCC(COc3cnccc3-2)C1] | E-25-[(3-fluoro-2-methoxyphenyl)amino]- 7,8,9,10,14,15,18,18a,19,20-decahydro-6H-7,11- methano-22,24-methenodipyrido[3,4-b:4′,3′- f][1,5,15]oxadiazacycloicosine-12,21(13H,23H)- dione |
| 119 | 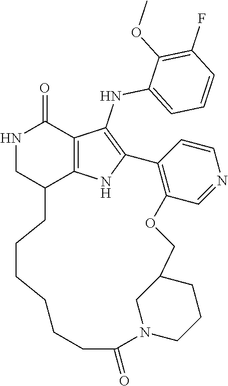 [SMILES: COc1c(F)cccc1Nc1c2[nH]c3c1C(=O)NCC3CCCCCCC(=O)N1CCCC(COc3cnccc3-2)C1] | 25-[(3-fluoro-2-methoxyphenyl)amino]- 7,8,9,10,14,15,16,17,18,18a,19,20-dodecahydro- 6H,7,11-methano-22,24-methenodipyrido[3,4- b:4′,3′-f][1,5,15]oxadiazacycloicosine- 12,21(13H,23H)-dione |
| 120 | 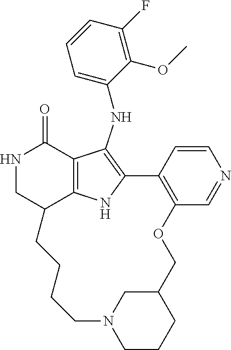 [SMILES: COc1c(F)cccc1Nc1c2[nH]c3c1C(=O)NCC3CCCCN1CCCC(COc3cnccc3-2)C1] | 22-(3-fluoro-2-methoxyanilino)- 7,8,9,10,13,14,15,15a,16,17-decahydro-6H,12H- 7,11-methano[19,21-(metheno)dipyrido[3,4- b:4′,3′-f][1,5,12]oxadiazacycloheptadecin- 18(20H)-one |
| 121 | 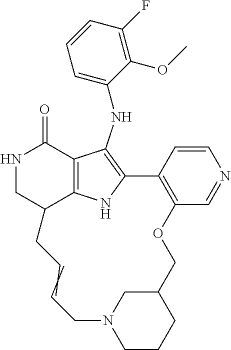 [SMILES: COc1c(F)cccc1Nc1c2[nH]c3c1C(=O)NCC3CC=CCN1CCCC(COc3cnccc3-2)C1] | 22-(3-fluoro-2-methoxyanilino)- 7,8,9,10,15,15a,16,17-octahydro-6H,12H-7,11- methano-19,21-(metheno)dipyrido[3,4-b:4′,3′- f][1,5,12]oxadiazacycloheptadecin-18(20H)-one |
| 122 | 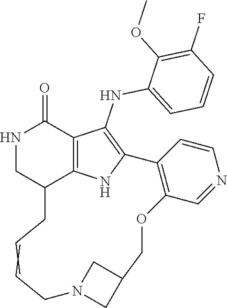 [SMILES: COc1c(F)cccc1Nc1c2[nH]c3c1C(=O)NCC3CC=CCN1CC(COc3cnccc3-2)C1] | 20-(3-fluoro-2-methoxyanilino)- 7,8,13,13a,14,15-hexahydro-6H,10H-7,9- methano-17,19-(metheno)dipyrido[3,4-b:4′,3′- f][1,5,12]oxadiazacyclopentadecin-16(18H)-one |
| 123 | 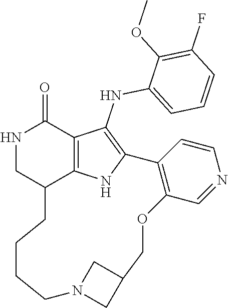 [SMILES: COc1c(F)cccc1Nc1c2[nH]c3c1C(=O)NCC3CCCCN1CC(COc3cnccc3-2)C1] | 20-(3-fluoro-2-methoxyanilino)- 7,8,11,12,13,13a,14,15-octahydro-6H,10H-7,9- methano-17,19-(metheno)dipyrido[3,4-b:4′,3′- f][1,5,12]oxadiazacyclopentadecin-16(18H)-one |
| 124 | 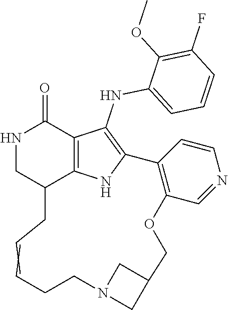 [SMILES: COc1c(F)cccc1Nc1c2[nH]c3c1C(=O)NCC3CC=CCCN1CC(COc3cnccc3-2)C1] | 21-(3-fluoro-2-methoxyanilino)- 7,8,10,11,14,14a,15,16-octahydro-6H-7,9- methano-18,20-(metheno)dipyrido[3,4-b:4′,3′- f][1,5,13]oxadiazacyclohexadecin-17(19H)-one |
| 125 | 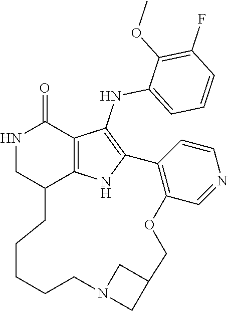 [SMILES: COc1c(F)cccc1Nc1c2[nH]c3c1C(=O)NCC3CCCCCN1CC(COc3cnccc3-2)C1] | 21-(3-fluoro-2-methoxyanilino)- 7,8,10,11,12,13,14,14a,15,16-decahydro-6H-7,9- methano-18,20-(metheno)dipyrido[3,4-b:4′,3′- f][1,5,13]oxadiazacyclohexadecin-17(19H)-one |
| 129 | 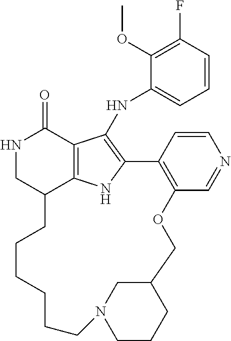 [SMILES: COc1c(F)cccc1Nc1c2[nH]c3c1C(=O)NCC3CCCCCCN1CCCC(COc3cnccc3-2)C1] | 24-(3-fluoro-2-methoxyanilino)- 7,8,9,10,13,14,15,16,17,17a,18,19-dodecahydro- 6H,12H-7,11-methano-21,23- (metheno)dipyrido[3,4-b:4′,3′- f][1,5,14]oxadiazacyclononadecin-20(22H)-one |
| 131 | 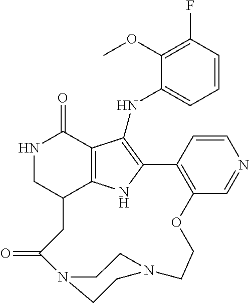 [SMILES: COc1c(F)cccc1Nc1c2[nH]c3c1C(=O)NCC3CC(=O)N1CCN(CCOc3cnccc3-2)CC1] | 20-(3-fluoro-2-methoxyanilino)- 6,7,9,10,13,13a,14,15-octahydro-12H-8,11- ethano-17,19-(metheno)dipyrido[3,4-j:4′,3′- n][1,4,7,12]oxatriazacyclopentadecine- 12,16(18H)-dione |
| 135 | 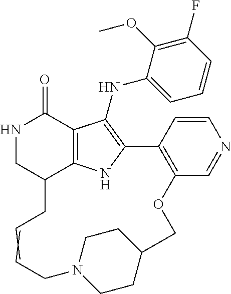 [SMILES: COc1c(F)cccc1Nc1c2[nH]c3c1C(=O)NCC3CC=CCN1CCC(CC1)COc1cnccc1-2] | 21-(3-fluoro-2-methoxyanilino)- 6,7,8,9,14,14a,15,16-octahydro-11H-7,10- ethano-18,20-(metheno)dipyrido[3,4-b:4′,3′- f][1,5,12]oxadiazacyclohexadecin-17(19H)-one |
| 136 | 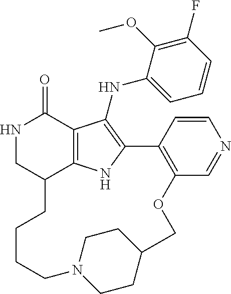 [SMILES: COc1c(F)cccc1Nc1c2[nH]c3c1C(=O)NCC3CCCCN1CCC(CC1)COc1cnccc1-2] | 21-(3-fluoro-2-methoxyanilino)- 6,7,8,9,12,13,14,14a,15,16-decahydro-11H-7,10- ethano-18,20-(metheno)dipyrido[3,4-b:4′,3′- f][1,5,12]oxadiazacyclohexadecin-17(19H)-one |
| 137 | 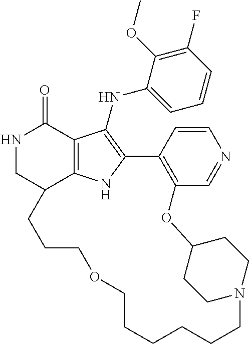 [SMILES: COc1c(F)cccc1Nc1c2[nH]c3c1C(=O)NCC3CCCOCCCCCCN1CCC(CC1)Oc1cnccc1-2] | 28-(3-fluoro-2-methoxyanilino)- 1,2,13,14,16,17,18,19,20,21,23,24,25,25a- tetradecahydro-12H-12,15-ethano-4,6- (metheno)dipyrido[3,4-b:4′,3′- f][1,11,5,18]dioxadiazacyclohenicosin-3(5H)- one |
| 139 | 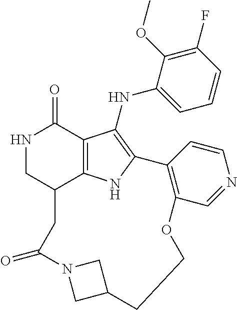 [SMILES: COc1c(F)cccc1Nc1c2[nH]c3c1C(=O)NCC3CC(=O)N1CC(CCOc3cnccc3-2)C1] | 19-(3-fluoro-2-methoxyanilino)- 6,7,8,9,12,12a,13,14-octahydro-11H-8,10- methano-16,18-(metheno)dipyrido[3,4-b:4′,3′- f][1,5,10]oxadiazacyclotetradecine-11,15(17H)- dione |
| 142 | 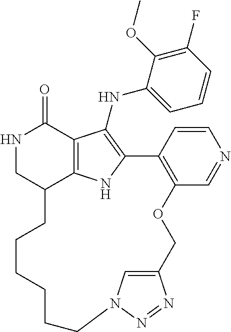 [SMILES: COc1c(F)cccc1Nc1c2[nH]c3c1C(=O)NCC3CCCCCCn1cc(nn1)COc1cnccc1-2] | 23-(3-fluoro-2-methoxyanilino)- 12,13,14,15,16,16a,17,18-octahydro-6H,11H- 10,7:20,22-di(metheno)dipyrido[3,4-m:4′,3′- q][1,4,5,6,15]oxatetraazacyclooctadecin- 19(21H)-one |
| 155 | 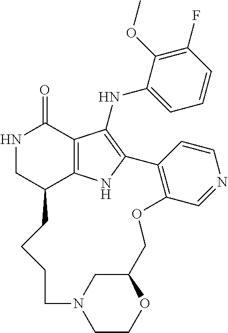 [SMILES: COc1c(F)cccc1Nc1c2[nH]c3c1C(=O)NC[C@@H]3CCCCN1CCO[C@H](COc3cnccc3-2)C1] | (7S,15aS)-22-(3-fluoro-2-methoxyanilino)- 6,7,9,10,13,14,15,15a,16,17-decahydro-12H- 7,11-methano-19,21-(metheno)dipyrido[3,4- l:4′,3′-p][1,4,7,14]dioxadiazacycloheptadecin- 18(20H)-one |
| 156 | 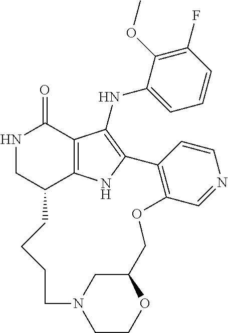 [SMILES: COc1c(F)cccc1Nc1c2[nH]c3c1C(=O)NC[C@H]3CCCCN1CCO[C@H](COc3cnccc3-2)C1] | (7S,15aR)-22-(3-fluoro-2-methoxyanilino)- 6,7,9,10,13,14,15,15a,16,17-decahydro-12H- 7,11-methano-19,21-(metheno)dipyrido[3,4- l:4′,3′-p][1,4,7,14]dioxadiazacycloheptadecin- 18(20H)-one |
| 157 | 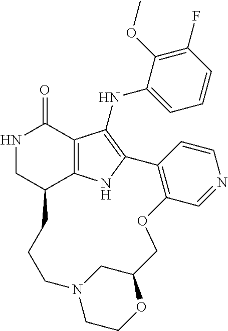 [SMILES: COc1c(F)cccc1Nc1c2[nH]c3c1C(=O)NC[C@@H]3CCCN1CCO[C@H](COc3cnccc3-2)C1] | (7S,14aS)-21-(3-fluoro-2-methoxyanilino)- 6,7,9,10,12,13,14,14a,15,16-decahydro-7,11- methano-18,20-(metheno)dipyrido[3,4-k:4′,3′- o][1,4,7,13]dioxadiazacyclohexadecin-17(19H)- one |
| 158 | 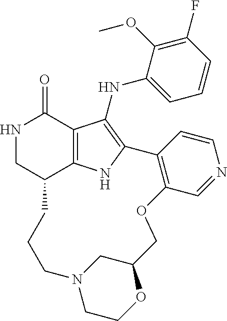 [SMILES: COc1c(F)cccc1Nc1c2[nH]c3c1C(=O)NC[C@H]3CCCN1CCO[C@H](COc3cnccc3-2)C1] | (7S,14aR)-21-(3-fluoro-2-methoxyanilino)- 6,7,9,10,12,13,14,14a,15,16-decahydro-7,11- methano-18,20-(metheno)dipyrido[3,4-k:4′,3′- o][1,4,7,13]dioxadiazacyclohexadecin-17(19H)- one |
| 160 | 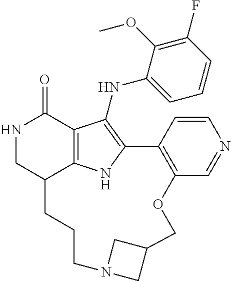 [SMILES: COc1c(F)cccc1Nc1c2[nH]c3c1C(=O)NCC3CCCN1CC(COc3cnccc3-2)C1] | 19-(3-fluoro-2-methoxyanilino)- 7,8,10,11,12,12a,13,14-octahydro-6H-7,9- methano-16,18-(metheno)dipyrido[3,4-b:4′,3′- f][1,5,11]oxadiazacyclotetradecin-15(17H)-one |
| 161 | 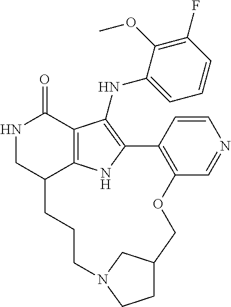 [SMILES: COc1c(F)cccc1Nc1c2[nH]c3c1C(=O)NCC3CCCN1CCC(COc3cnccc3-2)C1] | 20-(3-fluoro-2-methoxyanilino)- 6,7,8,9,11,12,13,13a,14,15-decahydro-7,10- methano-17,19-(metheno)dipyrido[3,4-b:4′,3′- f][1,5,11]oxadiazacyclopentadecin-16(18H)-one |
| 162 | 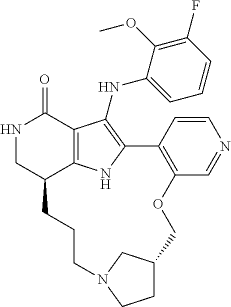 [SMILES: COc1c(F)cccc1Nc1c2[nH]c3c1C(=O)NC[C@@H]3CCCN1CC[C@@H](COc3cnccc3-2)C1] | (7R,13aS)-20-(3-fluoro-2-methoxyanilino)- 6,7,8,9,11,12,13,13a,14,15-decahydro-7,10- methano-17,19-(metheno)dipyrido[3,4-b:4′,3′- f][1,5,11]oxadiazacyclopentadecin-16(18H)-one |
| 163 | 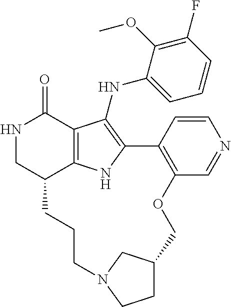 [SMILES: COc1c(F)cccc1Nc1c2[nH]c3c1C(=O)NC[C@H]3CCCN1CC[C@@H](COc3cnccc3-2)C1] | (7R,13aR)-20-(3-fluoro-2-methoxyanilino)- 6,7,8,9,11,12,13,13a,14,15-decahydro-7,10- methano-17,19-(metheno)dipyrido[3,4-b:4′,3′- f][1,5,11]oxadiazacyclopentadecin-16(18H)-one |
| 165 | 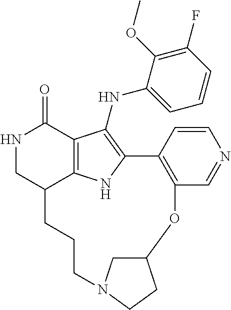 [SMILES: COc1c(F)cccc1Nc1c2[nH]c3c1C(=O)NCC3CCCN1CCC(C1)Oc1cnccc1-2] | 19-(3-fluoro-2-methoxyanilino)- 7,8,10,11,12,12a,13,14-octahydro-6H-6,9- methano-16,18-(metheno)dipyrido[3,4-b:4′,3′- f][1,5,11]oxadiazacyclotetradecin-15(17H)-one |
| 166 | 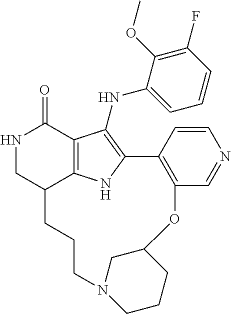 [SMILES: COc1c(F)cccc1Nc1c2[nH]c3c1C(=O)NCC3CCCN1CCCC(C1)Oc1cnccc1-2] | 20-(3-fluoro-2-methoxyanilino)- 6,7,8,9,11,12,13,13a,14,15-decahydro-6,10- methano-17,19-(metheno)dipyrido[3,4-b:4′,3′- f][1,5,11]oxadiazacyclopentadecin-16(18H)-one |
| 167 | 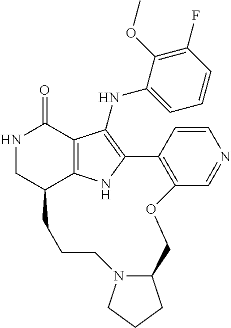 [SMILES: COc1c(F)cccc1Nc1c2[nH]c3c1C(=O)NC[C@@H]3CCCN1CCC[C@@H]1COc1cnccc1-2] | (12aR,19aS)-20-(3-fluoro-2-methoxyanilino)- 1,2,12a,13,14,15,17,18,19,19a-decahydro-12H- 4,6-(metheno)dipyrido[3,4-h:4′,3′-l]pyrrolo[2,1- c][1,4,10]oxadiazacyclotridecin-3(5H)-one |
| 168 | 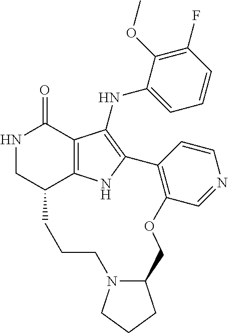 [SMILES: COc1c(F)cccc1Nc1c2[nH]c3c1C(=O)NC[C@H]3CCCN1CCC[C@@H]1COc1cnccc1-2] | (12aR,19aR)-20-(3-fluoro-2-methoxyanilino)- 1,2,12a,13,14,15,17,18,19,19a-decahydro-12H- 4,6-(metheno)dipyrido[3,4-h:4′,3′-l]pyrrolo[2,1- c][1,4,10]oxadiazacyclotridecin-3(5H)-one |
| 169 | 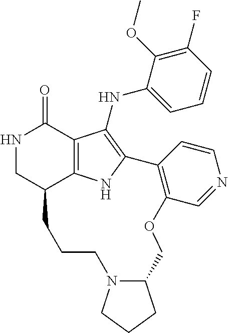 [SMILES: COc1c(F)cccc1Nc1c2[nH]c3c1C(=O)NC[C@@H]3CCCN1CCC[C@H]1COc1cnccc1-2] | (12aS,19aS)-20-(3-fluoro-2-methoxyanilino)- 1,2,12a,13,14,15,17,18,19,19a-decahydro-12H- 4,6-(metheno)dipyrido[3,4-h:4′,3′-l]pyrrolo[2,1- c][1,4,10]oxadiazacyclotridecin-3(5H)-one |
| 170 | 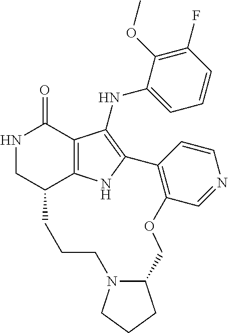 [SMILES: COc1c(F)cccc1Nc1c2[nH]c3c1C(=O)NC[C@H]3CCCN1CCC[C@H]1COc1cnccc1-2] | (12aS,19aR)-20-(3-fluoro-2-methoxyanilino)- 1,2,12a,13,14,15,17,18,19,19a-decahydro-12H- 4,6-(metheno)dipyrido[3,4-h:4′,3′-l]pyrrolo[2,1- c][1,4,10]oxadiazacyclotridecin-3(5H)-one |

or a pharmaceutically acceptable salt, stereoisomer, solvate, or tautomer thereof.

20. A pharmaceutical composition comprising the compound of claim 1 or a pharmaceutically acceptable salt, stereoisomer, solvate, or tautomer thereof, and a pharmaceutically acceptable carrier.

21. The pharmaceutical composition of claim 20, further comprising one or more additional pharmaceutically active agents.

22. A method of inhibiting of EGFR kinase activity in a cell, comprising contacting the cell with a compound of claim 1.

23. The method of claim 22, wherein the contacting is in vitro or in vivo.

24. A method for the treatment, mitigation, or prevention of a disease or disorder associated with EGFR kinase activity comprising administering to a subject in need thereof a compound of claim 1.

25. The method of claim 24, wherein the diseases are hyperproliferative diseases and/or disorders responsive to induction of cell death.

26. The method of claim 24, wherein the disease or disorder is selected from the group consisting of Lung Cancer; Lung Cancer Susceptibility 3 (LNCR3); Lung Squamous Cell Carcinoma; Gliosarcoma; Brain Stem Glioma; Adenocarcinoma; Colorectal Cancer; Glioblastoma; Breast Cancer; Squamous Cell Carcinoma; Small Cell Cancer of the Lung; Lung Squamous Cell Carcinoma; Inflammatory Skin and Bowel Disease, Neonatal, 2; Gastric Cancer; Prostate Cancer; Squamous Cell Carcinoma, Head and Neck; Pancreatic Cancer; Brain Cancer; Ovarian Cancer; Esophageal Cancer; Giant Cell Glioblastoma; Gliosarcoma; Lung Benign Neoplasm; Glioma; Exanthem; Endometrial Cancer; Adenoid Cystic Carcinoma; Bladder Cancer; Cowden Syndrome 1; Bronchiolo-Alveolar Adenocarcinoma; Oligodendroglioma; Hepatocellular Carcinoma; Lung Non-Squamous Non-Small Cell Carcinoma; High Grade Glioma; Glial Tumor; Laryngeal Squamous Cell Carcinoma; Renal Cell Carcinoma, Nonpapillary; Chronic Kidney Disease; Nasopharyngeal Carcinoma; Oral Squamous Cell Carcinoma; Lung Disease; Interstitial Lung Disease; Adenosquamous Lung Carcinoma; Bile Duct Cancer; Meningioma, Familial; Small Cell Carcinoma; Tumor Predisposition Syndrome; Pancreatic Adenocarcinoma; Diarrhea; Breast Ductal Carcinoma.

27. The method of claim 26, wherein the disease or disorder is lung cancer.

28. The method of any one of claims 23-27, wherein the subject is a mammal.

29. The method of claim 28, wherein the subject is a human.
